(19) United States
(12) Patent Application Publication
(10) Pub. No.: US 2025/0017914 A1
(43) Pub. Date: Jan. 16, 2025

(54) 6-AZA-QUINOLINE DERIVATIVES AND RELATED USES

(71) Applicant: Black Diamond Therapeutics, Inc., Cambridge, MA (US)

(72) Inventors: Pui Yee NG, Lexington, MA (US); Ivan JEWETT, Boston, MA (US); Fernando PADILLA, Arlington, MA (US)

(21) Appl. No.: 18/689,967

(22) PCT Filed: Sep. 9, 2022
(86) PCT No.: PCT/US2022/076164

§ 371 Date: Mar. 7, 2024

Related U.S. Application Data
(60) Provisional application No. 63/351,158, filed on Jun. 10, 2022.
(60) Provisional application No. 63/242,845, filed on Sep. 10, 2021.

(51) Int. Cl. A61K 31/444; A61K 31/4375; A61K 31/501; C07D 471/04; C07D 471/14; C07D 519/00

(52) U.S. Cl. CPC A61K 31/444; A61K 31/4375; A61K 31/501; C07D 471/04; C07D 471/14; C07D 519/00

(57) ABSTRACT

The present disclosure relates compounds of Formula (0):

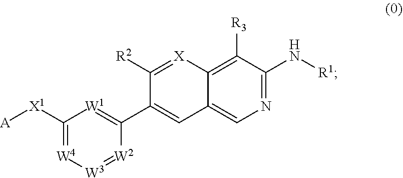

and pharmaceutically acceptable salts and stereoisomers thereof. The present disclosure also relates to methods of preparing the compounds, compositions comprising the compounds, and methods of using the compounds, e.g., in the treatment of cancer.

### RELATED APPLICATIONS

This application claims priority to, and the benefit of, U.S. Application Nos. 63/351,158, filed Jun. 10, 2022, and 63/242,845, filed Sep. 10, 2021, the entire contents of each of which are incorporated herein by reference.

### SEQUENCE LISTING

The Sequence Listing XML associated with this application is provided electronically in XML format and is hereby incorporated by reference into the specification. The name of the XML file containing the Sequence Listing XML is “ASET-019_001WO_SeqList.xml”. The XML file is 2,666 bytes, created on Sep. 8, 2022, and is being submitted electronically via USPTO Patent Center.

### BACKGROUND

Specific mutations in the human gene BRAF, which encodes for the protein B-Raf, are known to drive oncogenic activity in a variety of different cancers. Targeted inactivation of mutant B-Raf proteins by the administration of protein kinase inhibitors has been used to treat a number of different cancers in patients. However, there are subsets of patients administered these treatments that either fail to respond, eventually relapse or experience secondary lesions/pathway rebound. More specifically, many of the existing B-Raf-targeting kinase inhibitors either exhibit low specificity for B-Raf, leading to undesirable off-target effects, or only target a specific subset of BRAF/B-Raf mutation(s). Thus, there is a long-felt need in the art for new therapies that target specific oncogenic forms of B-Raf produced by mutations or alterations of the BRAF gene. The present disclosure provides compositions and methods for preventing or treating cancer in patients with oncogenic mutations in the BRAF gene and B-Raf protein.

### SUMMARY

In some aspects, the present disclosure provides a compound of Formula (0):

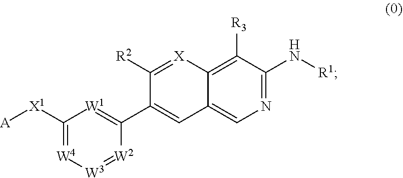

an isomer thereof, or a pharmaceutically acceptable salt thereof, wherein:
   X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4}
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆, alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R³ is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more RA;
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (I′):

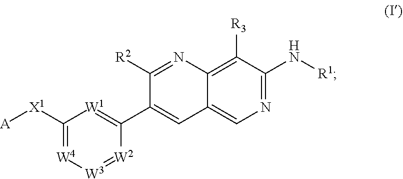

an isomer thereof, or a pharmaceutically acceptable salt thereof, wherein:
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R³ is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A})₂, C(═O)R^{A1}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (I):

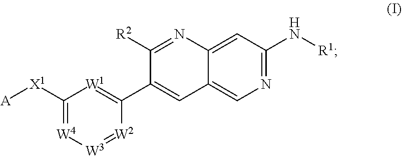

an isomer thereof, or a pharmaceutically acceptable salt thereof, wherein:
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆, alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (II′):

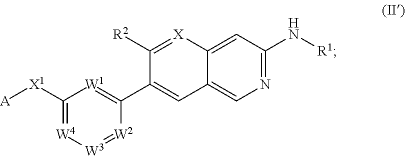

an isomer thereof, or a pharmaceutically acceptable salt thereof, wherein:
   X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆, alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides an isotopic derivative of a compound described herein.

In some aspects, the present disclosure provides a method of preparing a compound described herein.

In some aspects, the present disclosure provides a pharmaceutical composition comprising a compound described herein and one or more pharmaceutically acceptable carriers or excipients.

In some aspects, the present disclosure provides a method of treating or preventing cancer in a subject, the method comprising administering to the subject a therapeutically effective amount of a compound described herein.

In some aspects, the present disclosure provides a compound described herein for treating or preventing cancer in a subject.

In some aspects, the present disclosure provides a use of a compound described herein in the manufacture of a medicament for treating or preventing cancer in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to compounds, and pharmaceutically acceptable salts and stereoisomers thereof, useful in the treatment of cancers associated with B-Raf oncogenic activity, including methods of preparing the compounds, compositions comprising the compounds, and methods of using the compounds (e.g., in the treatment of cancer).

### Compounds of the Present Disclosure

In some aspects, the present disclosure provides a compound of Formula (0):

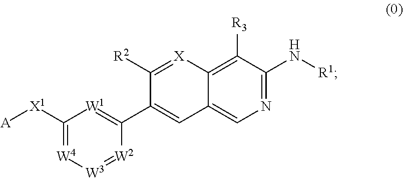

an isomer thereof, or a pharmaceutically acceptable salt thereof, wherein:
   X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R² is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (I′):

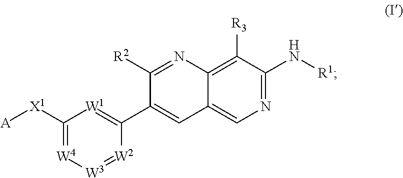

an isomer thereof, or a pharmaceutically acceptable salt thereof, wherein:
   W¹ is N or CR^{W1};
   R^{W1} is H, halogen, C₁-C₆ alkyl, C₂—C(alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆, alkenyl, or C₂-C₆ alkynyl;
   W⁴ is N or CR^{W4};
   R^{W4} is H, halogen, C₁-C₆ alkyl, C₂-C₆, alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R³ is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆, alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (I):

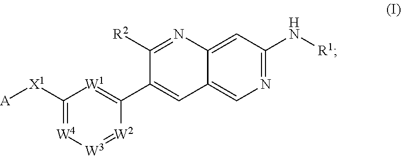

an isomer thereof, or a pharmaceutically acceptable salt thereof, wherein:
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —N^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A1})₂, C(═O)R^{A1}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (II′):

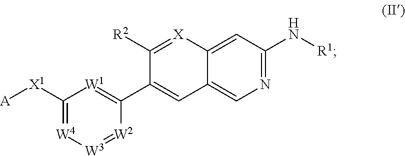

an isomer thereof, or a pharmaceutically acceptable salt thereof, wherein:
   X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —N^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A1})₂, C(═O)R^{A1}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

In some aspects, the present disclosure provides a compound of Formula (I):

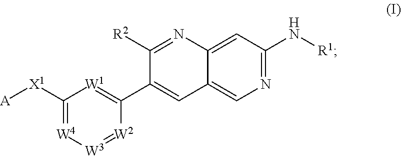

an isomer thereof, or a pharmaceutically acceptable salt thereof, wherein:
   W¹ is CR^{W1};
   R^{W1} is H, halogen, or C₁-C₆ alkyl;
   W² is N or CR^{W2};
   R^{W2} is H, halogen, or C₁-C₆ alkyl;
   W³ is N or CR^{W3};
   R^{W3} is H, or halogen;
   W⁴ is N or CR^{W4};
   R^{W4} is H, or halogen;
   R¹ is H, or C₁-C₆ alkyl, wherein the C₁-C₆ alkyl is optionally substituted with one or more R^{1a};
   each R^{1a} is cyano;
   R² is H, or cyano;
   X¹ is —NR^{X1}—*, —C(═O)NR^{X1}—*, —NR^{X1}C(═O)—*, or —NR^{X1}C(═NH)—*, wherein * denotes attachment to A;
   R^{X1} is H;
   A is C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl), wherein the C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl) is optionally substituted with one or more R^{A};
   each R^{A} independently is halogen, OH, OR^{A1}, NHR^{A1}, N(R^{A1})₂, C₁-C₆ alkyl, C₁-C₆ alkoxy, or C₃-C₈ cycloalkyl, wherein the C₁-C₆ alkyl, C₁-C₆ alkoxy, or C₃-C₈ cycloalkyl is optionally substituted with one or more R^{A1};
   each R^{A1} independently is halogen, cyano, oxo, OH, OR^{A2}, NH₂, or C₁-C₆ alkyl, wherein the C₁-C₆ alkyl is optionally substituted with one or more R^{A2}; and
   each R^{A2} independently is halogen, OH, or C₁-C₆ alkyl.

It is understood that, for a compound of the present disclosure, variables X, R^{X}, W¹, R^{W1}, W², R^{W2}, W³, R^{W3}, W⁴, R^{W4}, R¹, R^{1a}, R², R³, X¹, R^{X1}, R^{X1a}, A, R^{A}, R^{A1}, R^{A2}, R^{A3} can each be, where applicable, selected from the groups described herein, and any group described herein for any of variables X, R^{X}, W¹, R^{W1}, W², R^{W2}, W³, R^{W3}, W⁴, R^{W4}, R¹, R^{1a}, R², R³, X¹, R^{X1}, R^{X1a}, A, R^{A}, R^{A1}, R^{A2}, R^{A3} can be combined, where applicable, with any group described herein for one or more of the remainder of variables X, R^{X}, W¹, R^{W1}, W², R^{W2}, W³, R^{W3}, W⁴, R^{W4}, R¹, R^{1a}, R², R³, X¹, R^{X1}, R^{X1a}, A, R^{A}, R^{A1}, R^{A2}, R^{A3}.

### Variables X, R^{X}

In some embodiments, X is CR^{X} or N.

In some embodiments, X is N.

In some embodiments, X is CR^{X}. In some embodiments, X is CH. In some embodiments, X is C(CN). In some embodiments, X is CF.

In some embodiments, R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH.

In some embodiments, R^{X} is H, halogen, cyano, oxo, or OH.

In some embodiments, R^{X} is H.

In some embodiments, R^{X} is halogen. In some embodiments, R^{X} is fluorine. In some embodiments, R^{X} is chlorine. In some embodiments, R^{X} is bromine. In some embodiments, R^{X} is iodine.

In some embodiments, R^{X} is cyano.

In some embodiments, R^{X} is C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH.

In some embodiments, R^{X} is C₁-C₆ alkoxy, optionally substituted with one or more OH. In some embodiments, R^{X} is C₁ alkoxy, optionally substituted with one or more OH. In some embodiments, R^{X} is C₂ alkoxy, optionally substituted with one or more OH. In some embodiments, R^{X} is C₃ alkoxy, optionally substituted with one or more OH. In some embodiments, R^{X} is C₄ alkoxy, optionally substituted with one or more OH. In some embodiments, R^{X} is C₅ alkoxy, optionally substituted with one or more OH. In some embodiments, R^{X} is C₆ alkoxy, optionally substituted with one or more OH.

Variables W¹, R^{W1}, W², R^{W2}, W³, R^{W3}, W⁴, R^{W4}

In some embodiments, W¹ is N or CR^{W1}.

In some embodiments, W¹ is N.

In some embodiments, W¹ is CR^{W1}. In some embodiments, W¹ is CH.

In some embodiments, R^{W1} is H, halogen, C₁-C₆ alkyl, C₂-C₆ alkenyl, or C₂-C₆ alkynyl.

In some embodiments, R^{W1} is H or halogen,

In some embodiments, R^{W1} is H.

In some embodiments, R^{W1} is halogen. In some embodiments, R^{W1} is fluorine. In some embodiments, R^{W1} is chlorine. In some embodiments, R^{W1} is bromine. In some embodiments, R^{W1} is iodine.

In some embodiments, R^{W1} is C₁-C₆ alkyl, C₂-C₆, alkenyl, or C₂-C₆ alkynyl.

In some embodiments, R^{W1} is C₁-C₆, alkyl. In some embodiments, R^{W1} is C₁ alkyl. In some embodiments, R^{W1} is C₂ alkyl. In some embodiments, R^{W1} is C₃ alkyl. In some embodiments, R^{W1} is C₄ alkyl. In some embodiments, R^{W1} is C₅ alkyl. In some embodiments, R^{W1} is C₆ alkyl.

In some embodiments, R^{W1} is CH₃.

In some embodiments, W² is N or CR^{W2}.

In some embodiments, W² is N.

In some embodiments, W² is CR^{W2}. In some embodiments, W² is CH.

In some embodiments, W² is C(CH₃).

In some embodiments, R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen.

In some embodiments, R^{W2} is H or halogen.

In some embodiments, R^{W2} is H.

In some embodiments, R^{W2} is halogen. In some embodiments, R^{W2} is fluorine. In some embodiments, R^{W2} is chlorine. In some embodiments, R^{W2} is bromine. In some embodiments, R^{W2} is iodine.

In some embodiments, R^{W2} is C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen.

In some embodiments, R^{W2} is C₁-C₆ alkyl, C₂-C₆ alkenyl, or C₂-C₆ alkynyl.

In some embodiments, R^{W2} is C₁-C₆ alkyl. In some embodiments, R^{W2} is C₁ alkyl. In some embodiments, R^{W2} is C₂ alkyl. In some embodiments, R^{W2} is C₃ alkyl. In some embodiments, R^{W2} is C₄ alkyl. In some embodiments, R^{W2} is C₅ alkyl. In some embodiments, R^{W2} is C₆ alkyl.

In some embodiments, R² is CH₃.

In some embodiments, W³ is N or CR^{W3}.

In some embodiments, W³ is N.

In some embodiments, W³ is CR^{W3}. In some embodiments, W³ is CH.

In some embodiments, R^{W3} is H, halogen, C₁-C₆ alkyl, C₂-C₆ alkenyl, or C₂-C₆ alkynyl.

In some embodiments, R^{W3} is H or halogen.

In some embodiments, R^{W3} is H.

In some embodiments, R^{W3} is halogen. In some embodiments, R^{W3} is fluorine. In some embodiments, R^{W3} is chlorine. In some embodiments, R^{W} is bromine. In some embodiments, R^{W3} is iodine.

In some embodiments, R^{W3} is C₁-C₆ alkyl, C₂-C₆ alkenyl, or C₂-C₆ alkynyl.

In some embodiments, R^{W3} is C₁-C₆, alkyl. In some embodiments, R^{W3} is C₁ alkyl. In some embodiments, R^{W3} is C₂ alkyl. In some embodiments, R^{W3} is C₃ alkyl. In some embodiments, R^{W3} is C₄ alkyl. In some embodiments, R^{W3} is C₅ alkyl. In some embodiments, R^{W3} is C₆ alkyl.

In some embodiments, W⁴ is N or CR^{W4}.

In some embodiments, W⁴ is N.

In some embodiments, W⁴ is CR^{W4}. In some embodiments, W⁴ is CH.

In some embodiments, R^{W4} is H, halogen, C₁-C₆ alkyl, C₂-C₆ alkenyl, C₂-C₆ alkynyl, or S(C₁-C₆ alkyl).

In some embodiments, R^{W4} is H or halogen.

In some embodiments, R^{W4} is H.

In some embodiments, R^{W4} is halogen. In some embodiments, R^{W4} is fluorine. In some embodiments, R^{W4} is chlorine. In some embodiments, R^{W4} is bromine. In some embodiments, R^{W4} is iodine.

In some embodiments, R^{W4} is C₁-C₆ alkyl, C₂-C₆ alkenyl, C₂-C₆ alkynyl, or S(C₁-C₆ alkyl).

In some embodiments, R^{W4} is C₁-C₆ alkyl. In some embodiments, R⁴ is C₁ alkyl. In some embodiments, R^{W4} is C₂ alkyl. In some embodiments, R^{W4} is C₃ alkyl. In some embodiments, R^{W4} is C₄ alkyl. In some embodiments, R^{W4} is C₅ alkyl. In some embodiments, R^{W4} is C₆ alkyl.

In some embodiments, W¹ is CR^{W1}, W² is CR^{W2}, W³ is CR^{W3} and W⁴ is CR^{W4}.

In some embodiments, W¹ is CH, W² is CH, W³ is CH and W⁴ is CH.

In some embodiments, W¹ is CH, W² is C(CH₃), W³ is CH and W⁴ is CH.

In some embodiments, W¹ is CR^{W1}, W² is CR^{W2}, W³ is N and W⁴ is CR^{W4}.

In some embodiments, W¹ is CH, W² is C(CH₃), W³ is N and W⁴ is CH.

In some embodiments, W¹ is CR^{W1}, W² is N, W³ is N and W⁴ is CR^{W4}.

In some embodiments, W¹ is CR^{W1}, W² is CR², W³ is N and W⁴ is N.

Variables R¹, R^{1a}, R², R³

In some embodiments, R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a}.

In some embodiments, R¹ is H.

In some embodiments, R¹ is C₁-C₆ alkyl, wherein the C₁-C₆ alkyl is substituted with one or more R^{1a}.

In some embodiments, R¹ is C₁-C₆ alkyl. In some embodiments, R¹ is C₁ alkyl. In some embodiments, R¹ is C₂ alkyl. In some embodiments, R¹ is C₃ alkyl. In some embodiments, R¹ is C₄ alkyl. In some embodiments, R¹ is C₅ alkyl. In some embodiments, R¹ is C₆ alkyl.

In some embodiments, R¹ is CH₃.

In some embodiments, R¹ is CH₂CH₃.

In some embodiments, R^{1a} is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl.

In some embodiments, R^{1a} is halogen, cyano, oxo, OH or NH₂.

In some embodiments, R^{1a} is cyano.

In some embodiments, R^{1a} is NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl.

In some embodiments, R² is H, cyano, oxo, OH, C₁-C₆ alkyl, C₂-C₆ alkenyl, C₂-C₆ alkynyl, or C₁-C₆ alkoxy.

In some embodiments, R² is H, cyano, oxo, or OH.

In some embodiments, R² is H.

In some embodiments, R² is cyano.

In some embodiments, R² is OH.

In some embodiments, R² is C₁-C₆ alkyl, C₂-C₆ alkenyl, C₂-C₆ alkynyl or C₁-C₆ alkoxy.

In some embodiments, R² is H, halogen, cyano, oxo, OH, C₁-C₆ alkyl, C₂-C₆ alkenyl, C₂-C₆ alkynyl, or C₁-C₆ alkoxy.

In some embodiments, R³ is H, halogen, cyano, oxo, OH, C₁-C₆ alkyl, C₂-C₆ alkenyl, C₂-C₆ alkynyl, or C₁-C₆ alkoxy.

In some embodiments, R¹ and R³, together with the intervening atoms, form a 4- to 12-membered heterocycloalkyl optionally substituted with one or more oxo

Variables X¹, R^{X1}, R^{X1a}, A, R^{A}, R^{A1}, R^{A2}, R^{A3}

In some embodiments, X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A.

In some embodiments, X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A.

In some embodiments, is —NR^{X1}—*, wherein * denotes attachment to A. In some embodiments, is —NH—*, wherein * denotes attachment to A.

In some embodiments, X¹ is —C(═O)NR^{X1}—* or —NR^{X1}C(═O)—*, wherein * denotes attachment to A.

In some embodiments, X¹ is —C(═O)NR^{X1}—*, wherein * denotes attachment to A. In some embodiments, X¹ is —C(═O)NH—*, wherein * denotes attachment to A.

In some embodiments, X¹ is —NR^{X1}C(═O)—*, wherein * denotes attachment to A. In some embodiments, X¹ is —NHC(═O)—*, wherein * denotes attachment to A.

In some embodiments, X¹ is —NR^{X1}C(═NH)—*, wherein * denotes attachment to A. In some embodiments, X¹ is —NHC(═NH)—*, wherein * denotes attachment to A.

In some embodiments, R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a}.

In some embodiments, R^{X1} is H.

In some embodiments, R^{X1} is S(═O)₂R^{X1a}.

In some embodiments, R^{X1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a}.

In some embodiments, R^{X1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl.

In some embodiments, R^{X1} is C₁-C₆ alkyl. In some embodiments, R^{X1} is CH₃.

In some embodiments, R^{X1a} is halogen, C₁-C₆ alkyl, or 3- to 8-membered heterocycloalkyl, wherein the C₁-C₆ alkyl, or 3- to 8-membered heterocycloalkyl is optionally substituted with one or more halogen.

In some embodiments, R^{X1a} is halogen.

In some embodiments, R^{X1a} is C₁-C₆ alkyl, optionally substituted with one or more halogen.

In some embodiments, R^{X1a} is 3- to 8-membered heterocycloalkyl, optionally substituted with one or more halogen.

In some embodiments, A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A}.

In some embodiments, A is C₁-C₆ alkyl, C₃-C₈ cycloalkyl, 3- to 8-membered heterocycloalkyl, C₆-C₁₀ aryl, or 5- to 10-membered heteroaryl, wherein the C₁-C₆ alkyl, C₃-C₈ cycloalkyl, 3- to 8-membered heterocycloalkyl, C₆-C₁₀ aryl, or 5- to 10-membered heteroaryl is optionally substituted with one or more R^{A}.

In some embodiments, A is C₃-C₈ cycloalkyl, 3- to 8-membered heterocycloalkyl, C₆-C₁₀ aryl, or 5- to 10-membered heteroaryl, wherein the C₃-C₈ cycloalkyl, 3- to 8-membered heterocycloalkyl, C₆-C₁₀ aryl, or 5- to 10-membered heteroaryl is optionally substituted with one or more R^{A}.

In some embodiments, A is C₃-C₈ cycloalkyl, or 3- to 8-membered heterocycloalkyl, wherein the C₃-C₈ cycloalkyl, or 3- to 8-membered heterocycloalkyl is optionally substituted with one or more R^{A}.

In some embodiments, A is 3- to 8-membered heterocycloalkyl. In some embodiments, A is tetrahydropyranyl. In some embodiments, A is piperidinyl.

In some embodiments, A is 3- to 8-membered heterocycloalkyl optionally substituted with one or more R^{A}. In some embodiments, A is tetrahydropyranyl optionally substituted with one or more R^{A}. In some embodiments, A is piperidinyl optionally substituted with one or more R^{A}.

In some embodiments, A is C₆-C₁₀ aryl, or 5- to 10-membered heteroaryl, wherein the C₆-C₁₀ aryl, or 5- to 10-membered heteroaryl is optionally substituted with one or more R^{A}.

In some embodiments, A is C₆-C₁₀ aryl, optionally substituted with one or more R^{A}.

In some embodiments, A is C₆-C₁₀ aryl. In some embodiments, A is phenyl.

In some embodiments, A is phenyl optionally substituted with one or more R^{A}.

In some embodiments, A is 5- to 10-membered heteroaryl, optionally substituted with one or more R^{A}.

In some embodiments, A is 5- to 10-membered heteroaryl. In some embodiments, A is pyridyl. In some embodiments, A is triazolyl. In some embodiments, A is pyrazolyl. In some embodiments, A is imidazolyl. In some embodiments, A is oxazolyl. In some embodiments, A is imidazo[1,5-a]pyridyl. In some embodiments, A is 2,3-dihydrofuro[2,3-c]pyridyl. In some embodiments, A is 2,3-dihydrofuro[3,2-b]pyridyl. In some embodiments, A is 3,4-dihydro-1H-pyrano[3,4-c]pyridyl. In some embodiments, A is 4,5,6,7-tetrahydrobenzo[d]isoxazolyl.

In some embodiments, A is pyridyl optionally substituted with one or more R^{A}. In some embodiments, A is triazolyl optionally substituted with one or more R^{A}. In some embodiments, A is pyrazolyl optionally substituted with one or more R^{A}. In some embodiments, A is imidazolyl optionally substituted with one or more R^{A}. In some embodiments, A is oxazolyl optionally substituted with one or more R^{A}. In some embodiments, A is imidazo[1,5-a]pyridyl optionally substituted with one or more R^{A}. In some embodiments, A is 2,3-dihydrofuro[2,3-c]pyridyl optionally substituted with one or more R^{A}. In some embodiments, A is 2,3-dihydrofuro[3,2-b]pyridyl optionally substituted with one or more R^{A}. In some embodiments, A is 3,4-dihydro-1H-pyrano[3,4-c]pyridyl optionally substituted with one or more R^{A}. In some embodiments, A is 4,5,6,7-tetrahydrobenzo[d]isoxazolyl optionally substituted with one or more R^{A}.

In some embodiments, A is —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A}.

In some embodiments, A is —(C₁-C₆ alkyl)-(3- to 8-membered heterocycloalkyl), or —(C₁-C₆, alkyl)-(5- to 10-membered heteroaryl), wherein the —(C₁-C₆ alkyl)-(3- to 8-membered heterocycloalkyl), or —(C₁-C₆ alkyl)-(5- to 10-membered heteroaryl) is optionally substituted with one or more R^{A}.

In some embodiments, A is —(C₁-C₆ alkyl)-(3- to 8-membered heterocycloalkyl), optionally substituted with one or more R^{A}.

In some embodiments, A is —(C₁-C₆ alkyl)-(3- to 8-membered heterocycloalkyl). In some embodiments, A is —(C₁ alkyl)-(tetrahydropyranyl). In some embodiments, A is —(C₁ alkyl)-(piperidinyl).

In some embodiments, A is —(C₁ alkyl)-(tetrahydropyranyl) optionally substituted with one or more R^{A}. In some embodiments, A is —(C₁ alkyl)-(piperidinyl) optionally substituted with one or more R^{A}.

In some embodiments, A is —(C₁-C₆ alkyl)-(5- to 10-membered heteroaryl), optionally substituted with one or more R^{A}.

In some embodiments, A is —(C₁-C₆ alkyl)-(5- to 10-membered heteroaryl). In some embodiments, A is —(C₁ alkyl)-(triazolyl). In some embodiments, A is —(C₂ alkyl)-(triazolyl).

In some embodiments, A is —(C₁ alkyl)-(triazolyl) optionally substituted with one or more R^{A}. In some embodiments, A is —(C₂ alkyl)-(triazolyl) optionally substituted with one or more R^{A}.

In some embodiments, X¹ is —C(═O)NH—*, wherein * denotes attachment to A, and A is phenyl, optionally substituted with one or more R^{A}.

In some embodiments, X¹ is —NHC(═O)—*, wherein * denotes attachment to A, and A is phenyl, optionally substituted with one or more R^{A}.

In some embodiments, X¹ is —NHC(═NH)—*, wherein * denotes attachment to A, and A is phenyl, optionally substituted with one or more R^{A}.

In some embodiments, X¹ is —C(═O)NH—*, wherein * denotes attachment to A, and A is pyridyl, optionally substituted with one or more R^{A}.

In some embodiments, X¹ is —NHC(═O)—*, wherein * denotes attachment to A, and A is pyridyl, optionally substituted with one or more R^{A}.

In some embodiments, X¹ is —NHC(═NH)—*, wherein * denotes attachment to A, and A is pyridyl, optionally substituted with one or more R^{A}.

In some embodiments, X¹ is —C(═O)NH—*, wherein * denotes attachment to A, and A is triazolyl, optionally substituted with one or more R^{A}.

In some embodiments, X¹ is —NHC(═O)—*, wherein * denotes attachment to A, and A is triazolyl, optionally substituted with one or more R^{A}.

In some embodiments, X¹ is —NHC(═NH)—*, wherein * denotes attachment to A, and A is triazolyl, optionally substituted with one or more R^{A}.

In some embodiments, X¹ is —C(═O)NH—*, wherein * denotes attachment to A, and A is tetrahydropyranyl, optionally substituted with one or more R^{A}.

In some embodiments, X¹ is —NHC(═O)—*, wherein * denotes attachment to A, and A is tetrahydropyranyl, optionally substituted with one or more R^{A}.

In some embodiments, X¹ is —NHC(═NH)—*, wherein * denotes attachment to A, and A is tetrahydropyranyl, optionally substituted with one or more R^{A}.

In some embodiments, X¹ is —C(═O)NH—*, wherein * denotes attachment to A, and A is piperidinyl, optionally substituted with one or more R^{A}.

In some embodiments, X¹ is —NHC(═O)—*, wherein * denotes attachment to A, and A is piperidinyl, optionally substituted with one or more R^{A}.

In some embodiments, X¹ is —NHC(═NH)—*, wherein * denotes attachment to A, and A is piperidinyl, optionally substituted with one or more R^{A}.

In some embodiments, X¹ is —C(═O)NH—*, wherein * denotes attachment to A, and A is —(C₁ alkyl)-(tetrahydropyranyl), optionally substituted with one or more R^{A}.

In some embodiments, X¹ is —NHC(═O)—*, wherein * denotes attachment to A, and A is —(C₁ alkyl)-(tetrahydropyranyl), optionally substituted with one or more R^{A}.

In some embodiments, X¹ is —NHC(═NH)—*, wherein * denotes attachment to A, and A is —(C₁ alkyl)-(tetrahydropyranyl), optionally substituted with one or more R^{A}.

In some embodiments, X¹ is —C(═O)NH—*, wherein * denotes attachment to A, and A is —(C₁ alkyl)-(piperidinyl), optionally substituted with one or more R^{A}.

In some embodiments, X¹ is —NHC(═O)—*, wherein * denotes attachment to A, and A is —(C₁ alkyl)-(piperidinyl), optionally substituted with one or more R^{A}.

In some embodiments, X¹ is —NHC(═NH)—*, wherein * denotes attachment to A, and A is —(C₁ alkyl)-(piperidinyl), optionally substituted with one or more R^{A}.

In some embodiments, X¹ is —C(═O)NH—*, wherein * denotes attachment to A, and A is —(C₁ alkyl)-(triazolyl), optionally substituted with one or more R^{A}. In some embodiments, X¹ is —C(═O)NH—*, wherein * denotes attachment to A, and A is —(C₂ alkyl)-(triazolyl), optionally substituted with one or more R^{A}.

In some embodiments, X¹ is —NHC(═O)—*, wherein * denotes attachment to A, and A is —(C₁ alkyl)-(triazolyl), optionally substituted with one or more R^{A}. In some embodiments, X¹ is —NHC(═O)—*, wherein * denotes attachment to A, and A is —(C₂ alkyl)-(triazolyl), optionally substituted with one or more R^{A}.

In some embodiments, X¹ is —NHC(═NH)—*, wherein * denotes attachment to A, and A is —(C₁ alkyl)-(triazolyl), optionally substituted with one or more R^{A}. In some embodiments, X¹ is —NHC(═NH)—*, wherein * denotes attachment to A, and A is —(C₂ alkyl)-(triazolyl), optionally substituted with one or more R^{A}.

In some embodiments, R^{A} is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1}.

In some embodiments, R^{A} is halogen, cyano, oxo, OH, OR^{A1}, NH₂, NHR^{A1}, N(R^{A1})₂, or (═N)R^{A1}.

In some embodiments, R^{A} is halogen. In some embodiments, R^{A} is fluorine. In some embodiments, R^{A} is chlorine. In some embodiments, R^{A} is bromine. In some embodiments, R^{A} is iodine.

In some embodiments, R^{A} is cyano.

In some embodiments, R^{A} is OH.

In some embodiments, R^{A} is OR^{A1}. In some embodiments, R^{A} is O(C₁-C₆ alkyl), optionally substituted with one or more R^{A2}.

In some embodiments, R^{A} is NHR^{A1}.

In some embodiments, R^{A} is N(R^{A1})₂. In some embodiments, R^{A} is N(CH₃)₂.

In some embodiments, R^{A} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1}.

In some embodiments, R^{A} is C₁-C₆ alkyl. In some embodiments, R^{A} is Cₜ alkyl. In some embodiments, R^{A} is C₂ alkyl. In some embodiments, R^{A} is C₃ alkyl. In some embodiments, R^{A} is C₄ alkyl. In some embodiments, R^{A} is C₅ alkyl. In some embodiments, R^{A} is C₆ alkyl.

In some embodiments, R^{A} is C₁-C₆ alkyl, optionally substituted with one or more R^{A1}. In some embodiments, R^{A} is C₁ alkyl, optionally substituted with one or more R^{A1}. In some embodiments, R^{A} is C₂ alkyl, optionally substituted with one or more R^{A}. In some embodiments, R^{A} is C₃ alkyl, optionally substituted with one or more R^{A1}. In some embodiments, R^{A} is C₄ alkyl, optionally substituted with one or more R^{A1}. In some embodiments, R^{A} is C₅ alkyl, optionally substituted with one or more R^{A1}. In some embodiments, R^{A} is C₆ alkyl, optionally substituted with one or more R^{A1}.

In some embodiments, R^{A} is C₁-C₆ alkyl, optionally substituted with one or more halogen.

In some embodiments, R^{A} is C₁ alkyl, optionally substituted with one or more halogen.

In some embodiments, R^{A} is C₃ alkyl, optionally substituted with one or more halogen.

In some embodiments, R^{A} is CH₃.

In some embodiments, R^{A} is CF₃.

In some embodiments, R^{A} is C(CH₃)₂CN.

In some embodiments, R^{A} is C₁-C₆ alkoxy. In some embodiments, R^{A} is C₁ alkoxy. In some embodiments, R^{A} is C₂ alkoxy. In some embodiments, R^{A} is C₃ alkoxy. In some embodiments, R^{A} is C₄ alkoxy. In some embodiments, R^{A} is C₅ alkoxy. In some embodiments, R^{A} is C₆ alkoxy.

In some embodiments, R^{A} is C₁-C₆ alkoxy, optionally substituted with one or more R^{A1}. In some embodiments, R^{A} is C₁ alkoxy, optionally substituted with one or more R^{A1}. In some embodiments, R^{A} is C₂ alkoxy, optionally substituted with one or more R^{A1}. In some embodiments, R^{A} is C₃ alkoxy, optionally substituted with one or more R^{A1}. In some embodiments, R^{A} is C₄ alkoxy, optionally substituted with one or more R^{A1}. In some embodiments, R^{A} is C₅ alkoxy, optionally substituted with one or more R^{A1}. In some embodiments, R^{A} is C₆ alkoxy, optionally substituted with one or more R^{A1}.

In some embodiments, R^{A} is C₃-C₈ cycloalkyl. In some embodiments, R^{A} is C₃ cycloalkyl.

In some embodiments, R^{A} is C₄ cycloalkyl. In some embodiments, R^{A} is C₅ cycloalkyl. In some embodiments, R^{A} is C₆ cycloalkyl. In some embodiments, R^{A} is C₇ cycloalkyl. In some embodiments, R^{A} is C₈ cycloalkyl.

In some embodiments, R^{A} is C₃-C₈ cycloalkyl, optionally substituted with one or more R^{A1}. In some embodiments, R^{A} is C₃ cycloalkyl, optionally substituted with one or more R^{A1}. In some embodiments, R^{A} is C₄ cycloalkyl, optionally substituted with one or more R^{A}. In some embodiments, R^{A} is C₅ cycloalkyl, optionally substituted with one or more R^{A1}. In some embodiments, R^{A} is C₆ cycloalkyl, optionally substituted with one or more R^{A1}. In some embodiments, R^{A} is C₇ cycloalkyl, optionally substituted with one or more R^{A1}. In some embodiments, R^{A} is C₈ cycloalkyl, optionally substituted with one or more R^{A1}.

In some embodiments, R^{A1} is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}.

In some embodiments, R^{A1} is halogen, cyano, oxo, OH, OR^{A2}, NH₂, NHR^{A2}, N(R^{A2})₂, or C(═O)R^{A2}.

In some embodiments, R^{A1} is halogen. In some embodiments, R^{A1} is fluorine. In some embodiments, R^{A1} is chlorine. In some embodiments, R^{A1} is bromine. In some embodiments, R^{A1} is iodine.

In some embodiments, R^{A1} is cyano.

In some embodiments, R^{A1} is oxo.

In some embodiments, R^{A1} is OH.

In some embodiments, R^{A1} is OR^{A2}.

In some embodiments, R^{A1} is NH₂.

In some embodiments, R^{A1} is C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}.

In some embodiments, R^{A1} is C₁-C₆ alkyl. In some embodiments, R^{A1} is C₁ alkyl. In some embodiments, R^{A1} is C₂ alkyl. In some embodiments, R^{A1} is C₃ alkyl. In some embodiments, R^{A1} is C₄ alkyl. In some embodiments, R^{A1} is C₅ alkyl. In some embodiments, R^{A} is C₆ alkyl.

In some embodiments, R^{A1} is C₁-C₆ alkyl, optionally substituted with one or more R^{A}?. In some embodiments, R^{A1} is C₁ alkyl, optionally substituted with one or more R^{A2}. In some embodiments, R^{A1} is C₂ alkyl, optionally substituted with one or more R^{A2}. In some embodiments, R^{A1} is C₃ alkyl, optionally substituted with one or more R^{A2}. In some embodiments, R^{A1} is C₄ alkyl, optionally substituted with one or more R^{A2}. In some embodiments, R^{A1} is C₅ alkyl, optionally substituted with one or more R^{A2}. In some embodiments, R^{A1} is C₆ alkyl, optionally substituted with one or more R^{A2}.

In some embodiments, R^{A} is C₁-C₆ alkyl and R^{A1} is halogen. In some embodiments, R^{A} is C₁ alkyl and R^{A1} is fluorine.

In some embodiments, R^{A} is C₁-C₆ alkyl and R^{A1} is cyano. In some embodiments, R^{A} is C₁ alkyl and R^{A1} is cyano.

In some embodiments, R^{A2} is halogen, cyano, OH, NH₂, N(R^{A3})₂, C(═O)R^{A3}, C₁-C₆ alkyl, C₂-C₆ alkenyl, or C₂-C₆ alkynyl.

In some embodiments, R^{A2} is halogen, cyano, OH, NH₂, N(R^{A3})₂, or C(═O)R^{A3}.

In some embodiments, R^{A2} is halogen. In some embodiments, R^{A2} is fluorine. In some embodiments, R^{A2} is chlorine. In some embodiments, R^{A2} is bromine. In some embodiments, R^{A2} is iodine.

In some embodiments, R^{A2} is OH.

In some embodiments, R^{A2} is C₁-C₆ alkyl, C₂-C₆ alkenyl, or C₂-C₆ alkynyl.

In some embodiments, R^{A2} is C₁-C₆ alkyl. In some embodiments, R^{A2} is C₁ alkyl. In some embodiments, R^{A2} is C₂ alkyl. In some embodiments, R^{A2} is C₃ alkyl. In some embodiments, R^{A2} is C₄ alkyl. In some embodiments, R^{A2} is C₅ alkyl. In some embodiments, R^{A2} is C₆ alkyl.

In some embodiments, R^{A3} is C₁-C₆ alkyl, C₂-C₆ alkenyl, or C₂-C₆ alkynyl.

In some embodiments, the compound is of Formula (I-a), (I-b), (I-c), (I-d), (I-e), or (I-f):

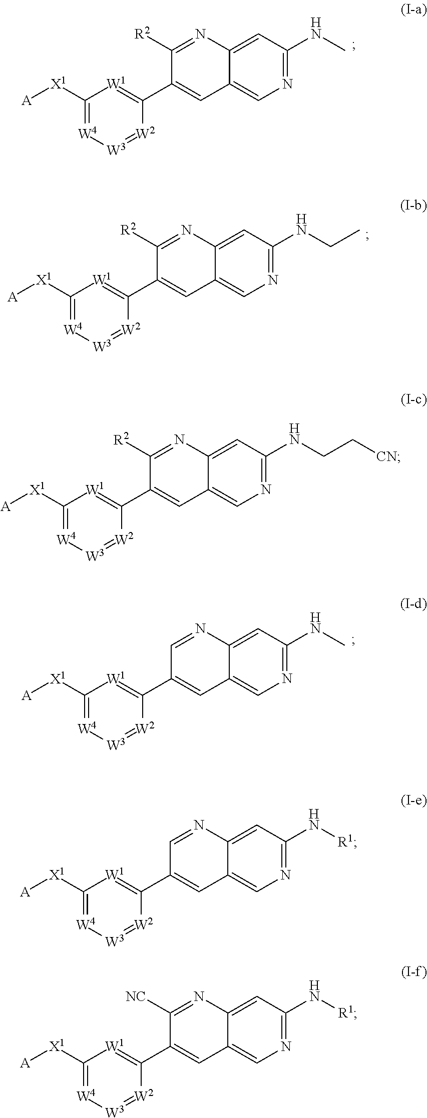

or a pharmaceutically acceptable salt or stereoisomer thereof.

In some embodiments, the compound is of Formula (I-g), (I-h), (I-i), (I-j), (I-k), (I-l), (I-m), (I-n), (I-o), (I-p), (I-q), (I-r), (I-s), or (I-t):

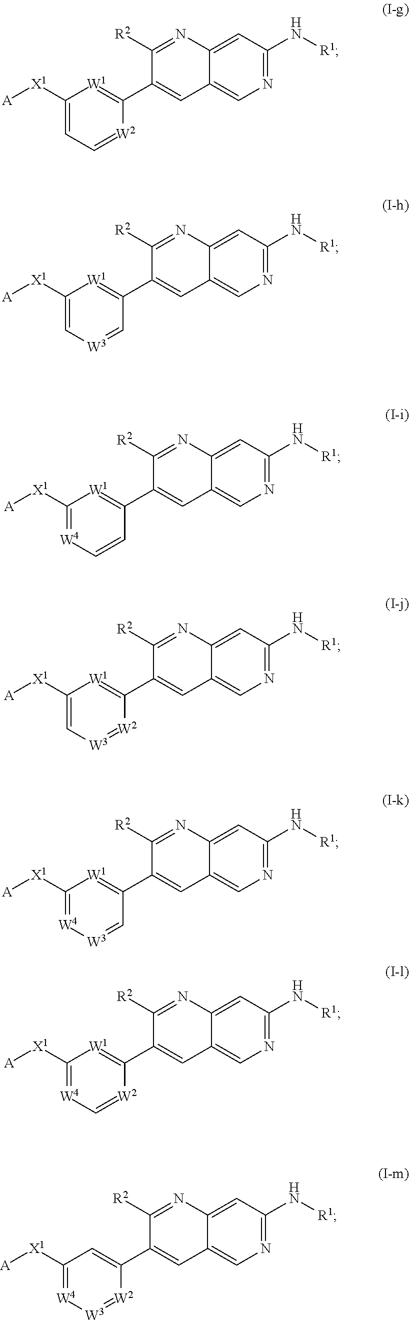

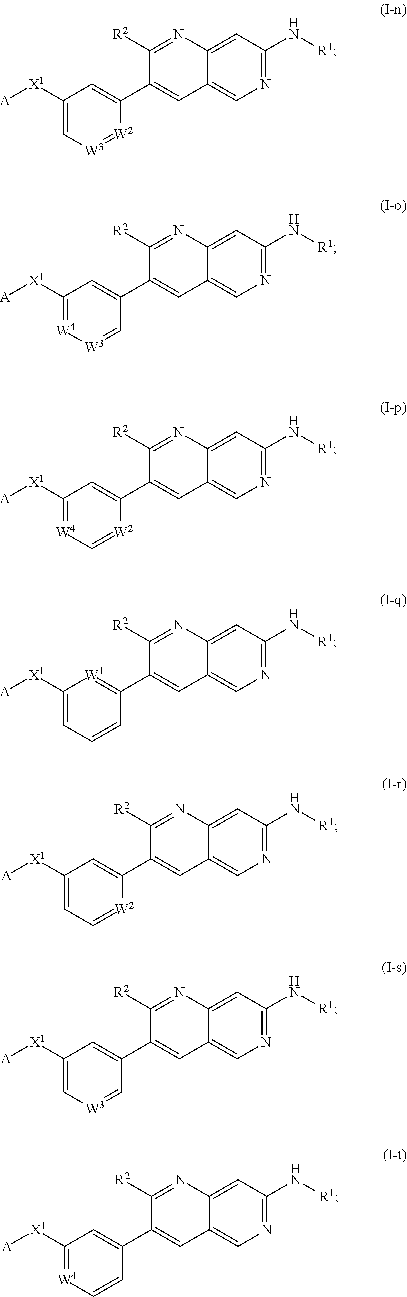

or a pharmaceutically acceptable salt or stereoisomer thereof.

In some embodiments, the compound is of Formula (I-u), (I-v), (I-w), or (I-x):

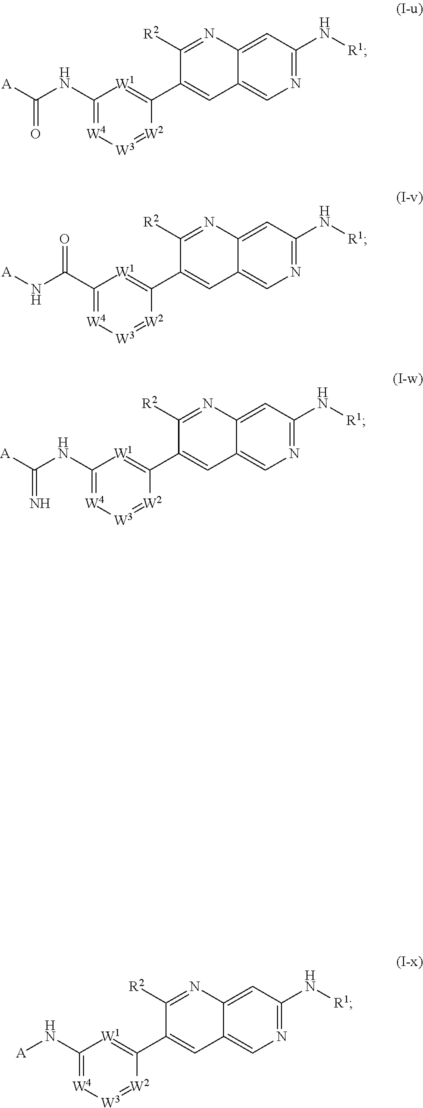

or a pharmaceutically acceptable salt or stereoisomer thereof.

In some embodiments, the compound is of Formula (I-a-i), (I-b-i), (I-c-i), (I-d-i), (I-e-i), or

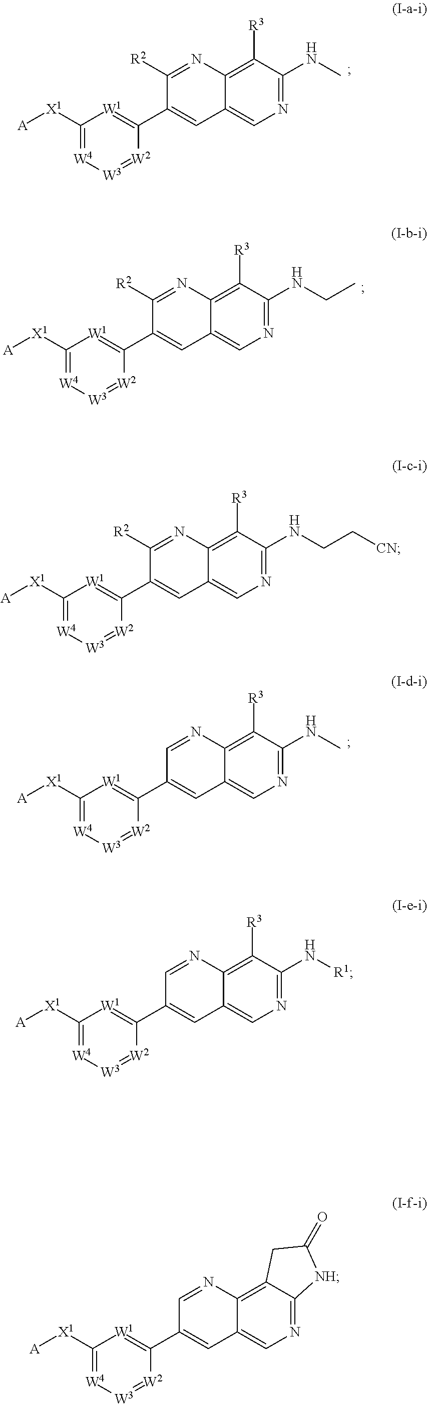

or a pharmaceutically acceptable salt or stereoisomer thereof.

In some embodiments, the compound is of Formula (I-g-i), (I-h-i), (I-i-i), (I-j-i), (I-k-i), (I-l-i), (I-m-i), (I-n-i), (I-o-i), (I-p-i), (I-q-i), (I-r-i), (I-s-i), or (I-t-i):

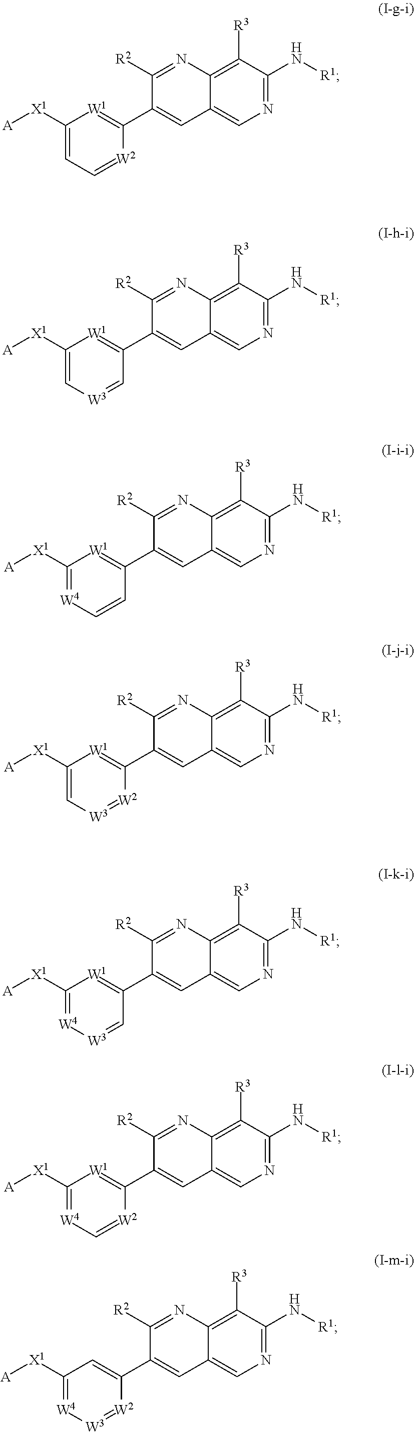

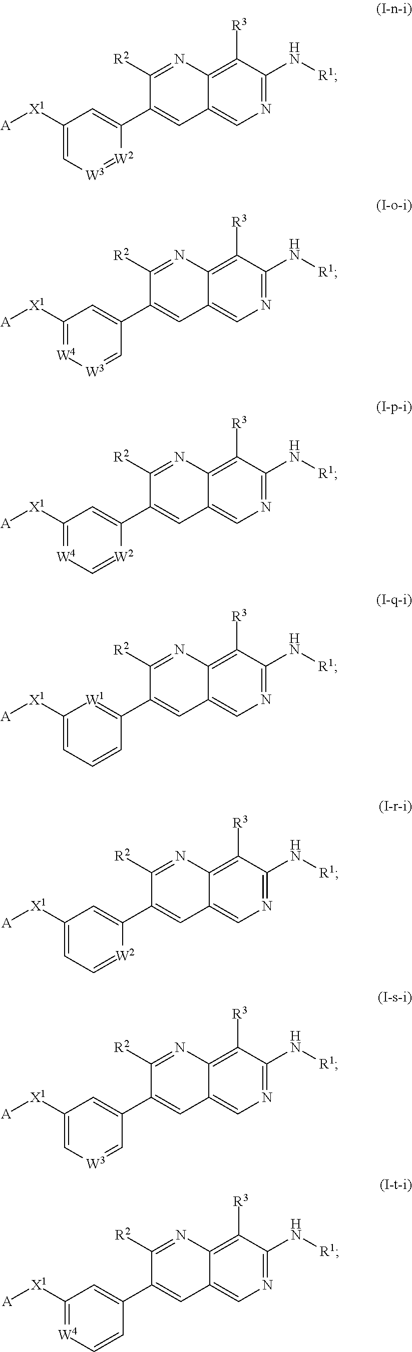

or a pharmaceutically acceptable salt or stereoisomer thereof.

In some embodiments, the compound is of Formula (I-u-i), (I-v-i), (I-w-i), or (I-x-i):

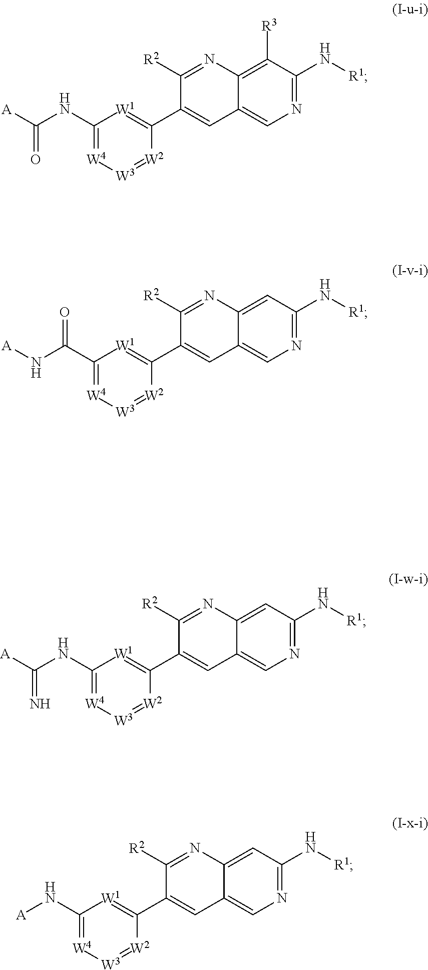

or a pharmaceutically acceptable salt or stereoisomer thereof.

In some embodiments, the compound is of Formula (I-g-ii), (I-h-ii), (I-i-ii), (I-j-ii), (I-k-ii), (I-l-ii), (I-m-ii), (I-n-ii), (I-o-ii), (I-p-ii), (I-q-ii), (I-r-ii), (I-s-ii), or (I-t-ii):

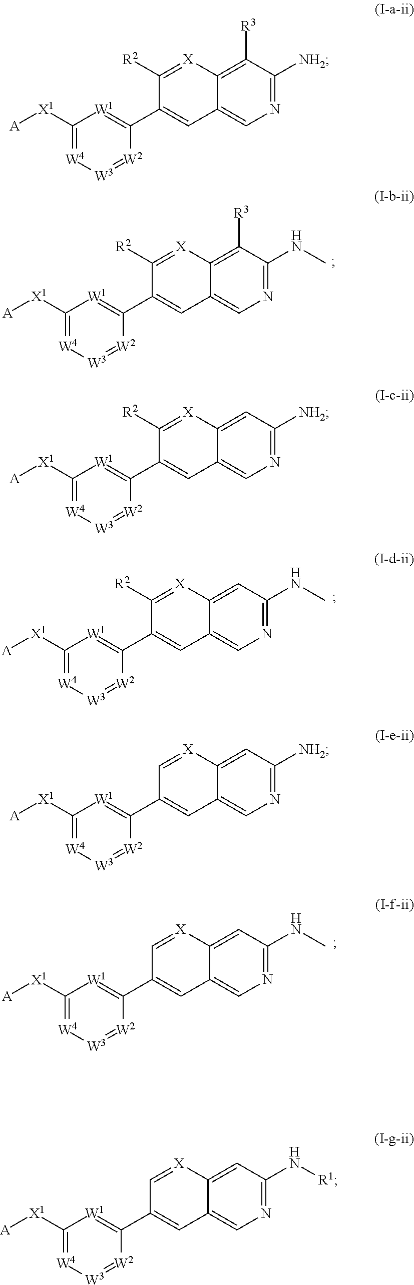

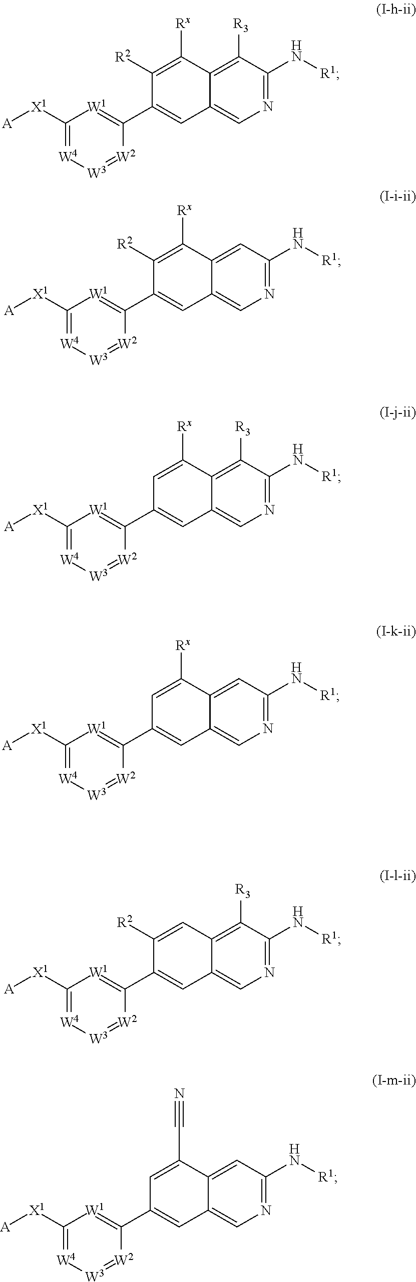

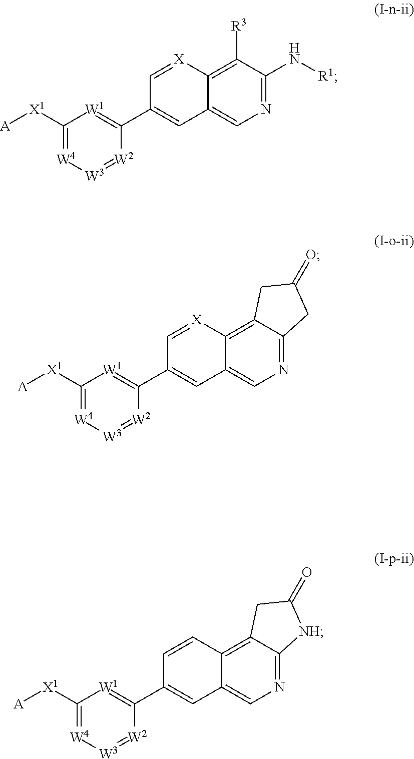

or a pharmaceutically acceptable salt or stereoisomer thereof.

In some embodiments, the compound is of Formula (I-q-ii), (I-r-ii), (I-s-ii), (I-t-ii), (I-u-ii), (I-v-ii) (I-w-ii), (I-x-ii), (I-y-ii), (I-z-ii), (I-aa-ii), (I-bb-ii), (I-cc-ii), or (I-dd-ii):

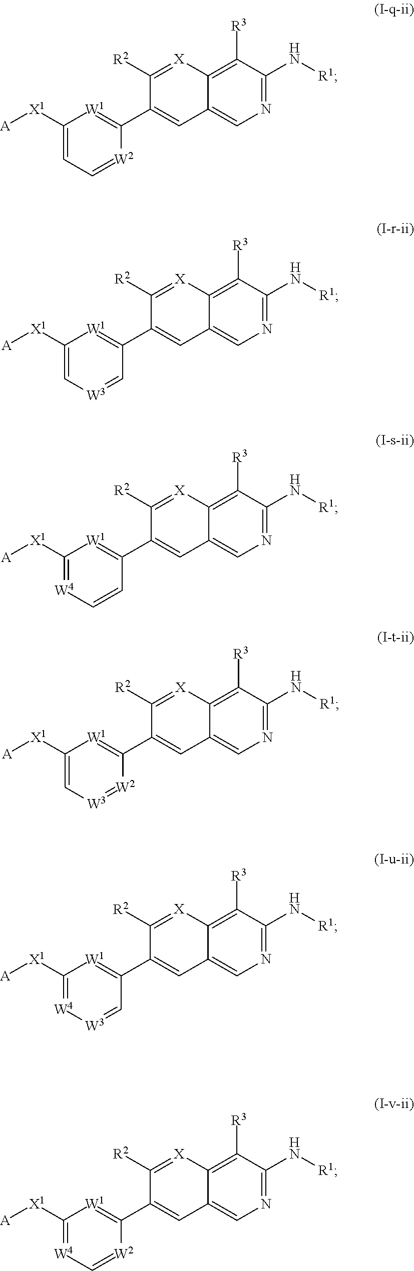

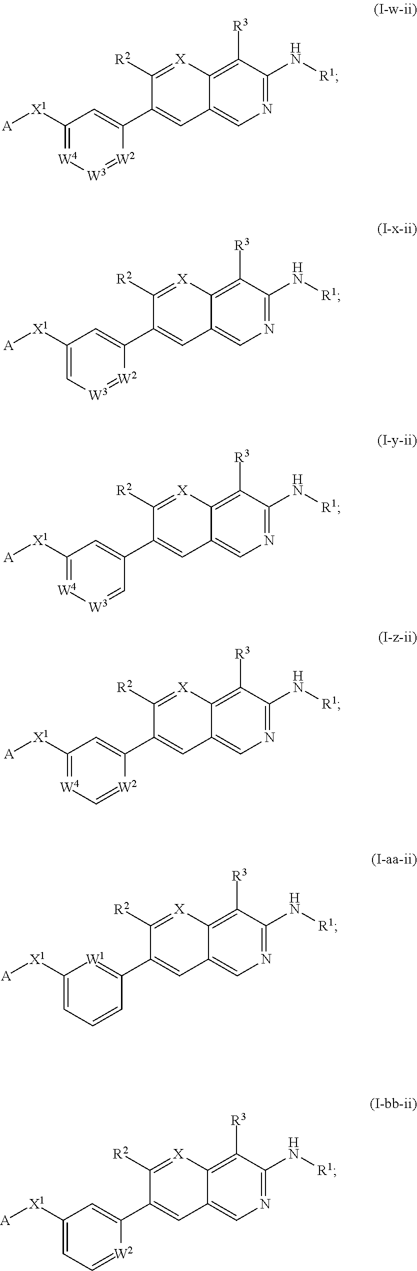

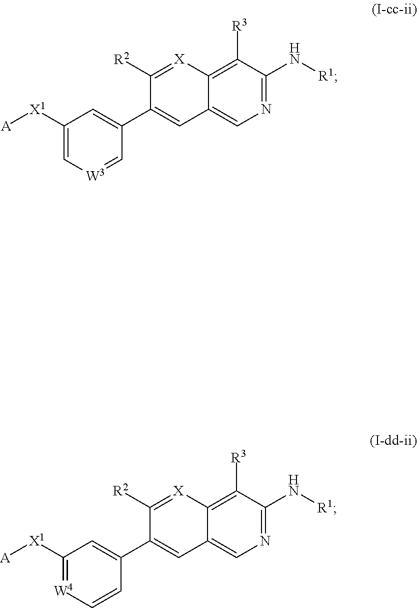

or a pharmaceutically acceptable salt or stereoisomer thereof.

In some embodiments, the compound is of Formula (I-ee-ii), (I-f-ii), (I-gg-ii), or (I-hh-ii):

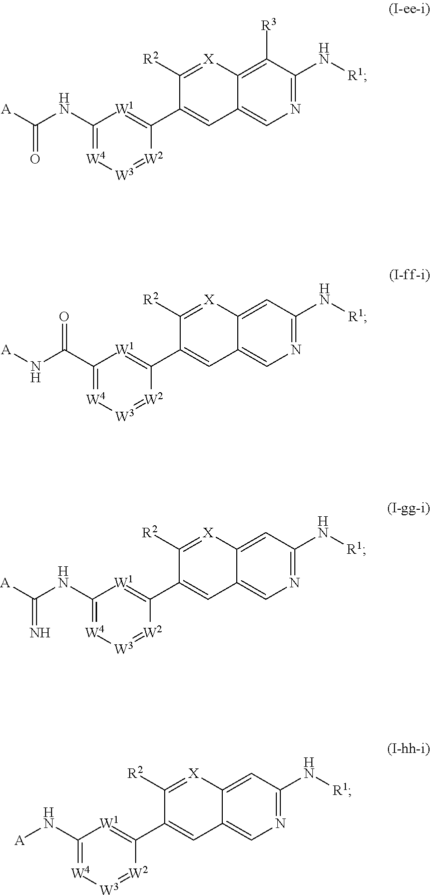

or a pharmaceutically acceptable salt or stereoisomer thereof.

In some embodiments, the compound is a compound described in Table I or II, or a pharmaceutically acceptable salt or stereoisomer thereof.

In some embodiments, the compound is a compound described in Table I or II, or a pharmaceutically acceptable salt thereof.

In some embodiments, the compound is a compound described in Table I or II.

In some embodiments, the compound is a compound described in Table II, or a pharmaceutically acceptable salt or stereoisomer thereof

In some embodiments, the compound is a compound described in Table II, or a pharmaceutically acceptable salt thereof.

In some embodiments, the compound is a compound described in Table II.

In some embodiments, the compound is a compound described in Table I, or a pharmaceutically acceptable salt or stereoisomer thereof.

In some embodiments, the compound is a compound described in Table I, or a pharmaceutically acceptable salt thereof.

In some embodiments, the compound is a compound described in Table I.

TABLE I

| Compound | |
|---|---|
| No. | Structure |
| 1 | 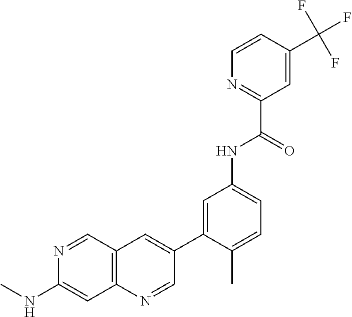 [SMILES: CNc1cc2ncc(-c3cc(NC(=O)c4cc(C(F)(F)F)ccn4)ccc3C)cc2cn1] |
| 2 | 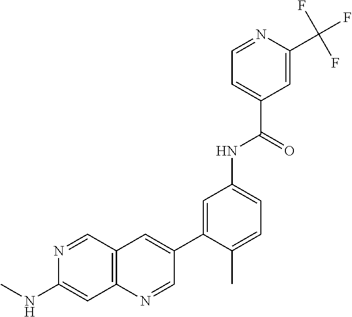 [SMILES: CNc1cc2ncc(-c3cc(NC(=O)c4ccnc(C(F)(F)F)c4)ccc3C)cc2cn1] |
| 3 | 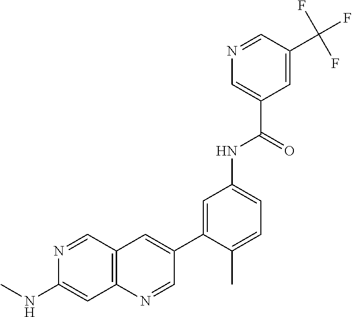 [SMILES: CNc1cc2ncc(-c3cc(NC(=O)c4cncc(C(F)(F)F)c4)ccc3C)cc2cn1] |
| 4 | 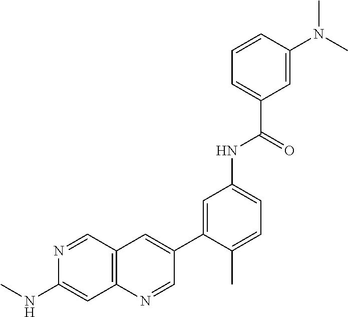 |
| 5 | 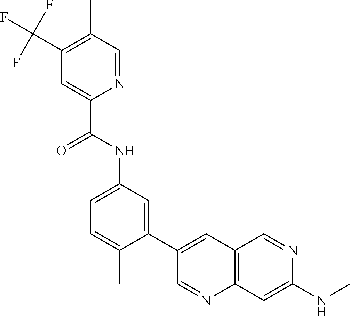 [SMILES: CNc1cc2ncc(-c3cc(NC(=O)c4cc(C(F)(F)F)c(C)cn4)ccc3C)cc2cn1] |
| 6 | 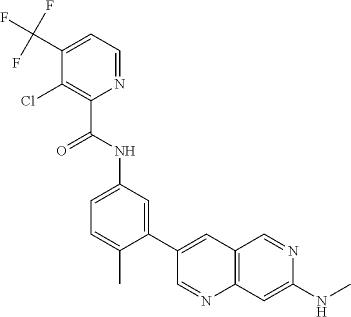 [SMILES: CNc1cc2ncc(-c3cc(NC(=O)c4nccc(C(F)(F)F)c4Cl)ccc3C)cc2cn1] |
| 7 | 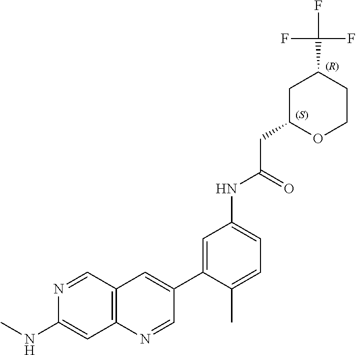 |
| 8 | 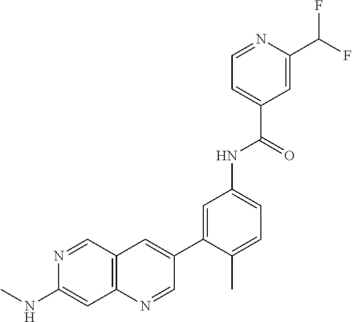 [SMILES: CNc1cc2ncc(-c3cc(NC(=O)c4ccnc(C(F)F)c4)ccc3C)cc2cn1] |
| 9 | 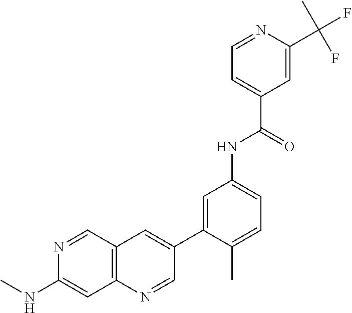 [SMILES: CNc1cc2ncc(-c3cc(NC(=O)c4ccnc(C(C)(F)F)c4)ccc3C)cc2cn1] |
| 10 | 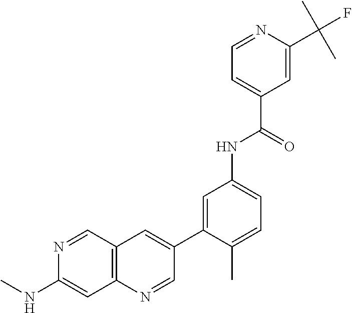 [SMILES: CNc1cc2ncc(-c3cc(NC(=O)c4ccnc(C(C)(C)F)c4)ccc3C)cc2cn1] |
| 11 | 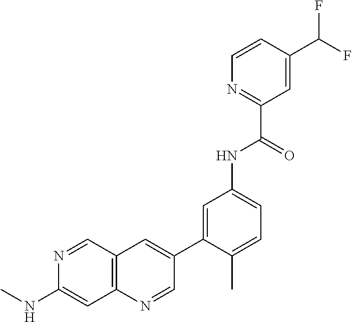 [SMILES: CNc1cc2ncc(-c3cc(NC(=O)c4cc(C(F)F)ccn4)ccc3C)cc2cn1] |
| 12 | 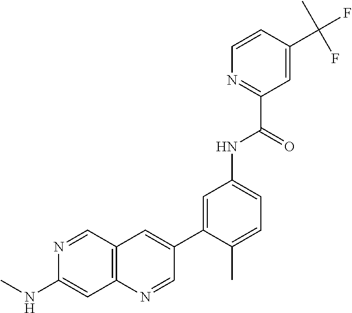 [SMILES: CNc1cc2ncc(-c3cc(NC(=O)c4cc(C(C)(F)F)ccn4)ccc3C)cc2cn1] |
| 13 | 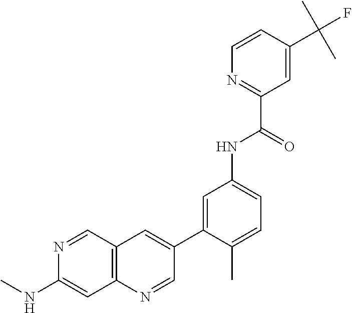 [SMILES: CNc1cc2ncc(-c3cc(NC(=O)c4cc(C(C)(C)F)ccn4)ccc3C)cc2cn1] |
| 14 | 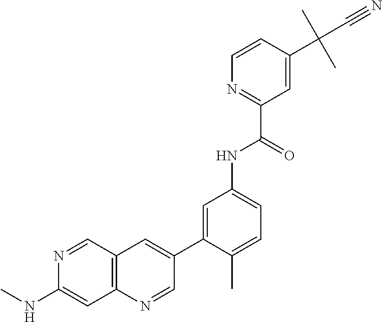 [SMILES: CNc1cc2ncc(-c3cc(NC(=O)c4cc(C(C)(C)C#N)ccn4)ccc3C)cc2cn1] |
| 15 | 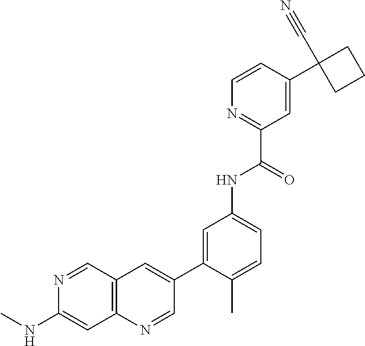 [SMILES: CNc1cc2ncc(-c3cc(NC(=O)c4cc(C5(C#N)CCC5)ccn4)ccc3C)cc2cn1] |
| 16 | 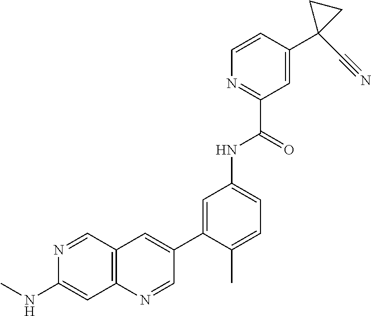 [SMILES: CNc1cc2ncc(-c3cc(NC(=O)c4cc(C5(C#N)CC5)ccn4)ccc3C)cc2cn1] |
| 17 | 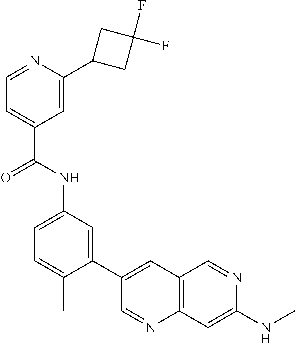 [SMILES: CNc1cc2ncc(-c3cc(NC(=O)c4ccnc(C5CC(F)(F)C5)c4)ccc3C)cc2cn1] |
| 18 | 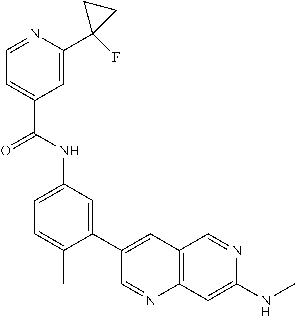 [SMILES: CNc1cc2ncc(-c3cc(NC(=O)c4ccnc(C5(F)CC5)c4)ccc3C)cc2cn1] |
| 19 | 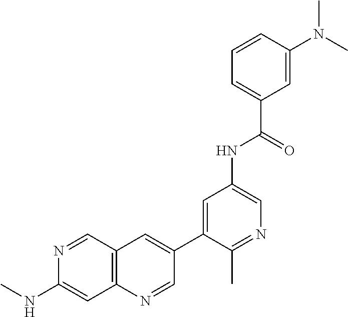 |
| 20 | 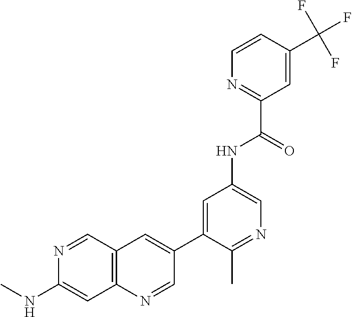 [SMILES: CNc1cc2ncc(-c3cc(NC(=O)c4cc(C(F)(F)F)ccn4)cnc3C)cc2cn1] |
| 21 | 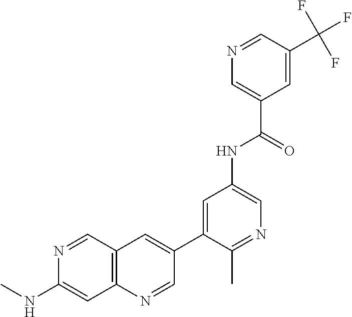 [SMILES: CNc1cc2ncc(-c3cc(NC(=O)c4cncc(C(F)(F)F)c4)cnc3C)cc2cn1] |
| 22 | 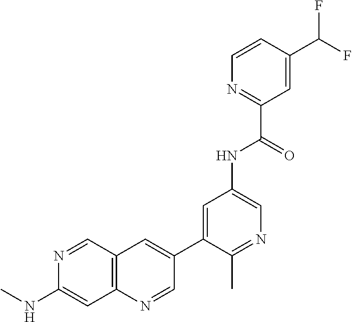 [SMILES: CNc1cc2ncc(-c3cc(NC(=O)c4cc(C(F)F)ccn4)cnc3C)cc2cn1] |
| 23 | 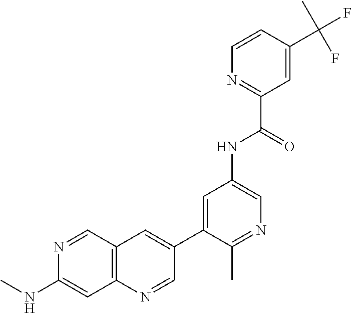 [SMILES: CNc1cc2ncc(-c3cc(NC(=O)c4cc(C(C)(F)F)ccn4)cnc3C)cc2cn1] |
| 24 | 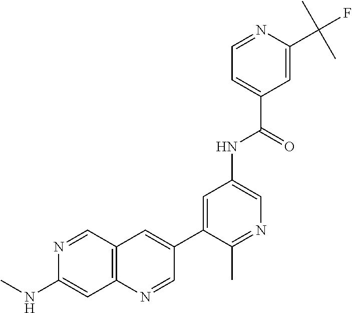 [SMILES: CNc1cc2ncc(-c3cc(NC(=O)c4ccnc(C(C)(C)F)c4)cnc3C)cc2cn1] |
| 25 | 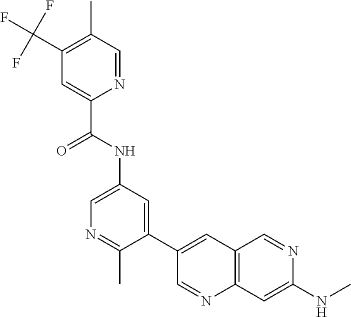 [SMILES: CNc1cc2ncc(-c3cc(NC(=O)c4cc(C(F)(F)F)c(C)cn4)cnc3C)cc2cn1] |
| 26 | 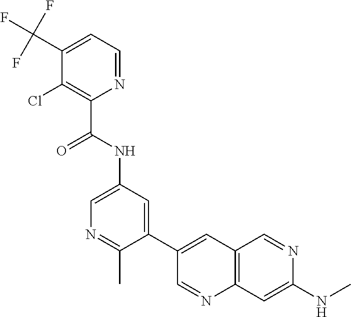 [SMILES: CNc1cc2ncc(-c3cc(NC(=O)c4nccc(C(F)(F)F)c4Cl)cnc3C)cc2cn1] |
| 27 | 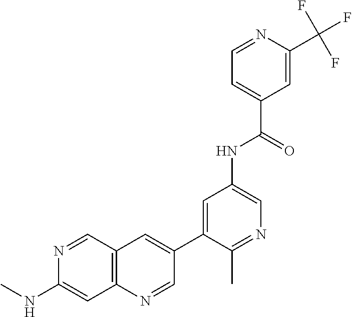 [SMILES: CNc1cc2ncc(-c3cc(NC(=O)c4ccnc(C(F)(F)F)c4)cnc3C)cc2cn1] |
| 28 | 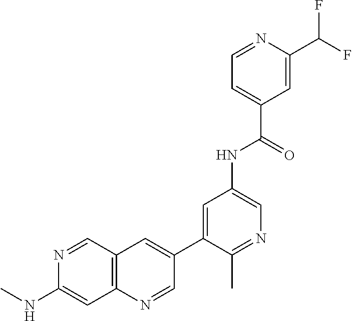 [SMILES: CNc1cc2ncc(-c3cc(NC(=O)c4ccnc(C(F)F)c4)cnc3C)cc2cn1] |
| 29 | 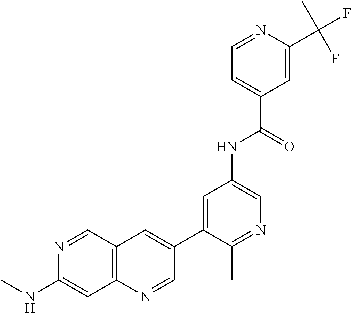 [SMILES: CNc1cc2ncc(-c3cc(NC(=O)c4ccnc(C(C)(F)F)c4)cnc3C)cc2cn1] |
| 30 | 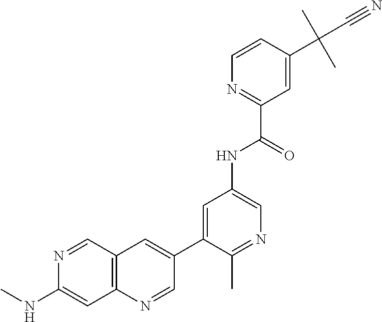 [SMILES: CNc1cc2ncc(-c3cc(NC(=O)c4cc(C(C)(C)C#N)ccn4)cnc3C)cc2cn1] |
| 31 | 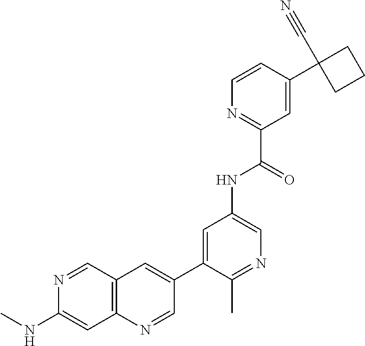 [SMILES: CNc1cc2ncc(-c3cc(NC(=O)c4cc(C5(C#N)CCC5)ccn4)cnc3C)cc2cn1] |
| 32 | 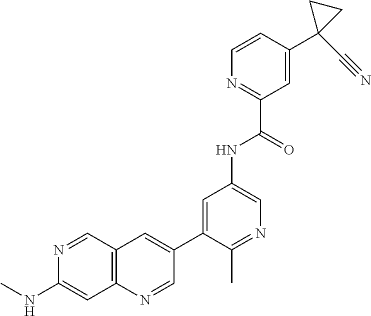 [SMILES: CNc1cc2ncc(-c3cc(NC(=O)c4cc(C5(C#N)CC5)ccn4)cnc3C)cc2cn1] |
| 33 | 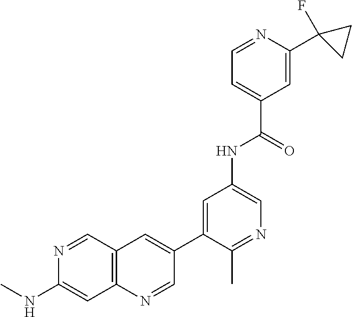 [SMILES: CNc1cc2ncc(-c3cc(NC(=O)c4ccnc(C5(F)CC5)c4)cnc3C)cc2cn1] |
| 34 | 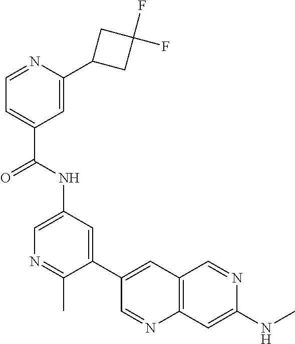 [SMILES: CNc1cc2ncc(-c3cc(NC(=O)c4ccnc(C5CC(F)(F)C5)c4)cnc3C)cc2cn1] |
| 35 | 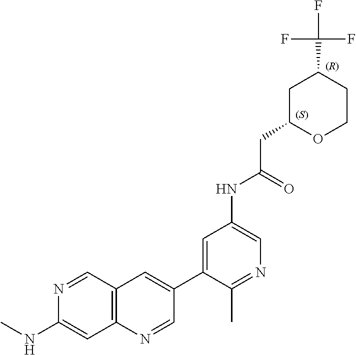 |
| 36 | 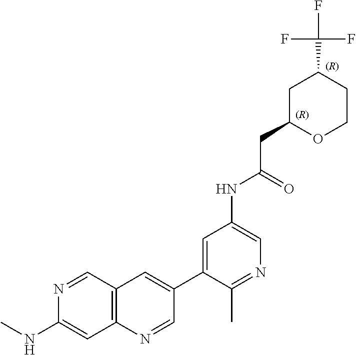 |
| 37 | 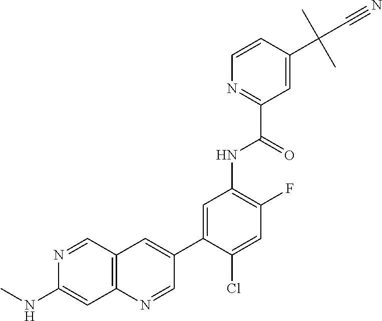 [SMILES: CNc1cc2ncc(-c3cc(NC(=O)c4cc(C(C)(C)C#N)ccn4)c(F)cc3Cl)cc2cn1] |
| 38 | 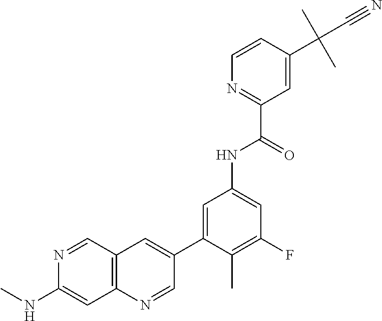 [SMILES: CNc1cc2ncc(-c3cc(NC(=O)c4cc(C(C)(C)C#N)ccn4)cc(F)c3C)cc2cn1] |
| 39 | 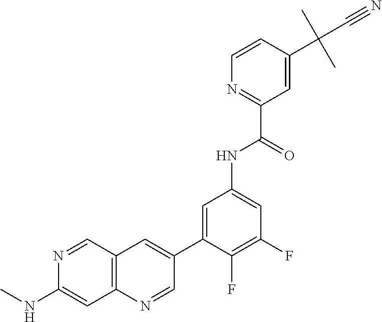 [SMILES: CNc1cc2ncc(-c3cc(NC(=O)c4cc(C(C)(C)C#N)ccn4)cc(F)c3F)cc2cn1] |
| 40 | 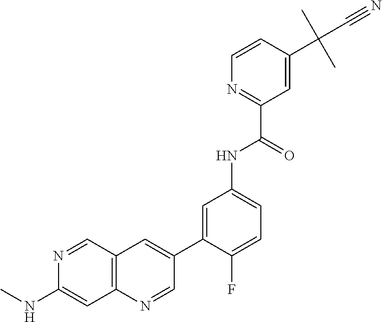 [SMILES: CNc1cc2ncc(-c3cc(NC(=O)c4cc(C(C)(C)C#N)ccn4)ccc3F)cc2cn1] |
| 41 | 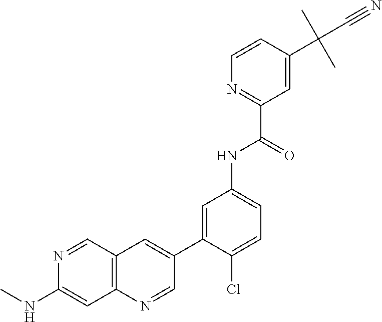 [SMILES: CNc1cc2ncc(-c3cc(NC(=O)c4cc(C(C)(C)C#N)ccn4)ccc3Cl)cc2cn1] |
| 42 | 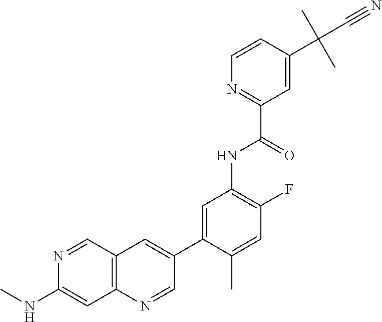 [SMILES: CNc1cc2ncc(-c3cc(NC(=O)c4cc(C(C)(C)C#N)ccn4)c(F)cc3C)cc2cn1] |
| 43 | 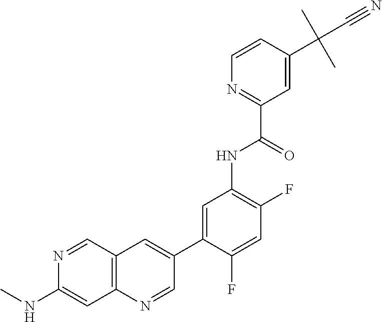 [SMILES: CNc1cc2ncc(-c3cc(NC(=O)c4cc(C(C)(C)C#N)ccn4)c(F)cc3F)cc2cn1] |
| 44 | 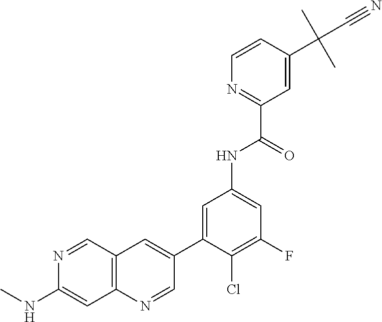 [SMILES: CNc1cc2ncc(-c3cc(NC(=O)c4cc(C(C)(C)C#N)ccn4)cc(F)c3Cl)cc2cn1] |
| 45 | 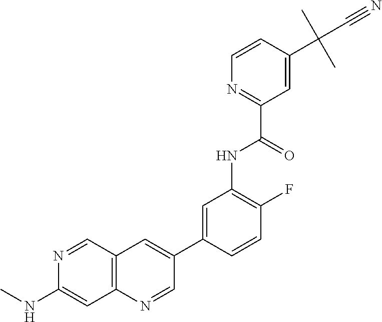 [SMILES: CNc1cc2ncc(-c3ccc(F)c(NC(=O)c4cc(C(C)(C)C#N)ccn4)c3)cc2cn1] |
| 46 | 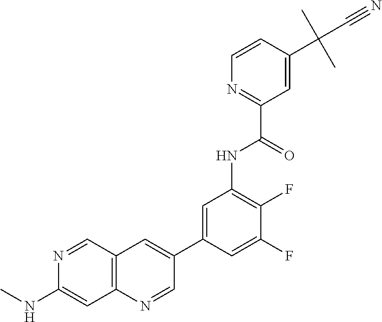 [SMILES: CNc1cc2ncc(-c3cc(F)c(F)c(NC(=O)c4cc(C(C)(C)C#N)ccn4)c3)cc2cn1] |
| 47 | 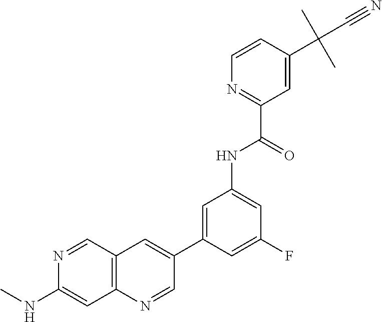 [SMILES: CNc1cc2ncc(-c3cc(F)cc(NC(=O)c4cc(C(C)(C)C#N)ccn4)c3)cc2cn1] |
| 48 | 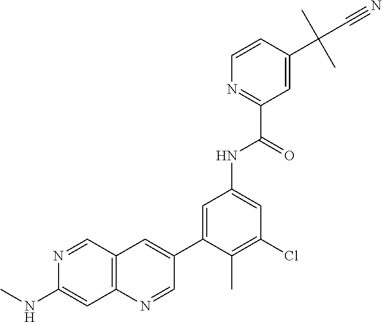 [SMILES: CNc1cc2ncc(-c3cc(NC(=O)c4cc(C(C)(C)C#N)ccn4)cc(Cl)c3C)cc2cn1] |
| 49 | 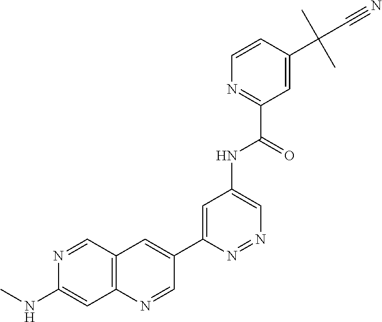 [SMILES: CNc1cc2ncc(-c3cc(NC(=O)c4cc(C(C)(C)C#N)ccn4)cnn3)cc2cn1] |
| 50 | 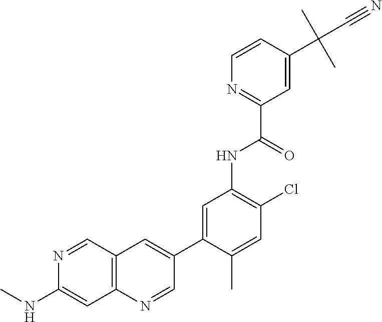 [SMILES: CNc1cc2ncc(-c3cc(NC(=O)c4cc(C(C)(C)C#N)ccn4)c(Cl)cc3C)cc2cn1] |
| 51 | 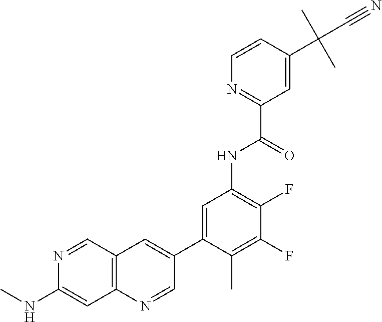 [SMILES: CNc1cc2ncc(-c3cc(NC(=O)c4cc(C(C)(C)C#N)ccn4)c(F)c(F)c3C)cc2cn1] |
| 52 | 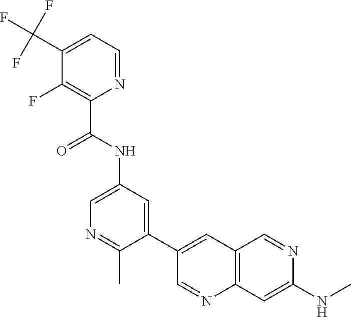 [SMILES: CNc1cc2ncc(-c3cc(NC(=O)c4nccc(C(F)(F)F)c4F)cnc3C)cc2cn1] |
| 53 | 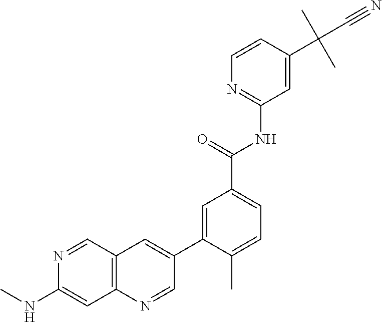 [SMILES: CNc1cc2ncc(-c3cc(C(=O)Nc4cc(C(C)(C)C#N)ccn4)ccc3C)cc2cn1] |
| 54 | 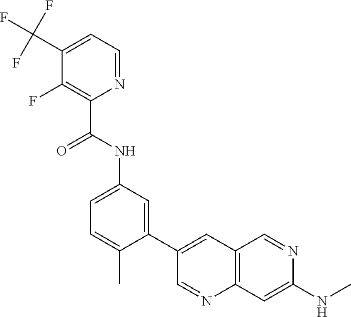 [SMILES: CNc1cc2ncc(-c3cc(NC(=O)c4nccc(C(F)(F)F)c4F)ccc3C)cc2cn1] |
| 55 | 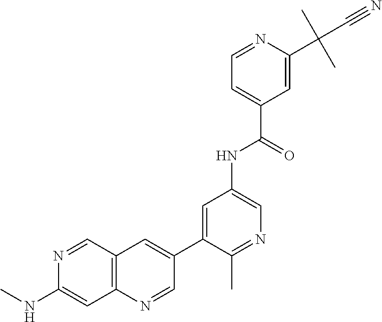 [SMILES: CNc1cc2ncc(-c3cc(NC(=O)c4ccnc(C(C)(C)C#N)c4)cnc3C)cc2cn1] |
| 56 | 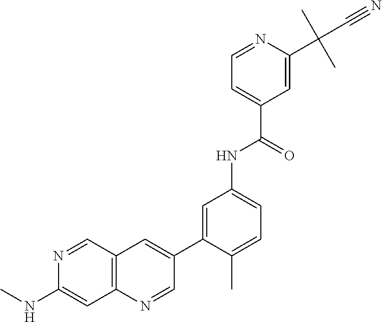 [SMILES: CNc1cc2ncc(-c3cc(NC(=O)c4ccnc(C(C)(C)C#N)c4)ccc3C)cc2cn1] |
| 57 | 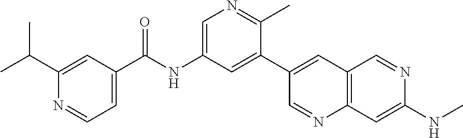 [SMILES: CNc1cc2ncc(-c3cc(NC(=O)c4ccnc(C(C)C)c4)cnc3C)cc2cn1] |
| 58 | 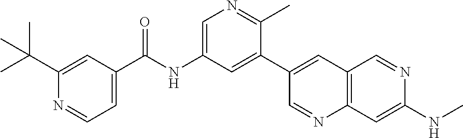 [SMILES: CNc1cc2ncc(-c3cc(NC(=O)c4ccnc(C(C)(C)C)c4)cnc3C)cc2cn1] |
| 59 | 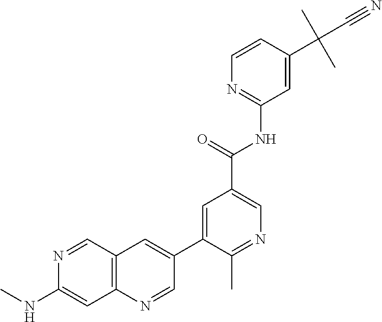 [SMILES: CNc1cc2ncc(-c3cc(C(=O)Nc4cc(C(C)(C)C#N)ccn4)cnc3C)cc2cn1] |
| 60 | 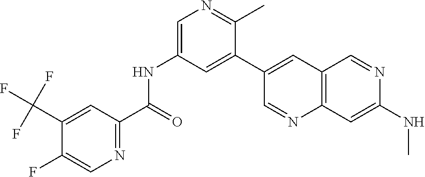 [SMILES: CNc1cc2ncc(-c3cc(NC(=O)c4cc(C(F)(F)F)c(F)cn4)cnc3C)cc2cn1] |
| 61 | 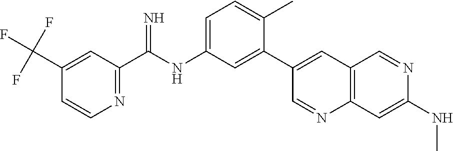 [SMILES: CNc1cc2ncc(-c3cc(NC(=N)c4cc(C(F)(F)F)ccn4)ccc3C)cc2cn1] |
| 62 | 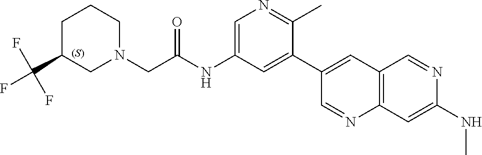 |
| 63 | 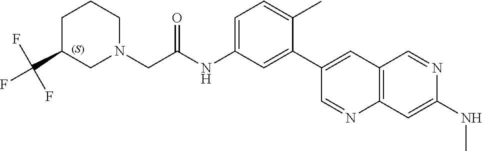 |
| 64 | 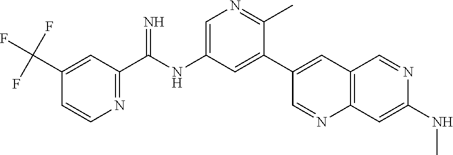 [SMILES: CNc1cc2ncc(-c3cc(NC(=N)c4cc(C(F)(F)F)ccn4)cnc3C)cc2cn1] |
| 65 | 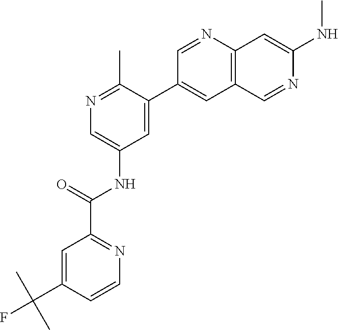 [SMILES: CNc1cc2ncc(-c3cc(NC(=O)c4cc(C(C)(C)F)ccn4)cnc3C)cc2cn1] |
| 66 | 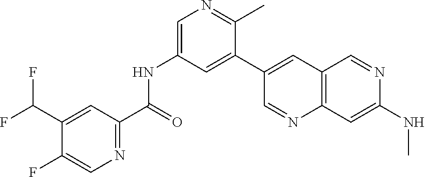 [SMILES: CNc1cc2ncc(-c3cc(NC(=O)c4cc(C(F)F)c(F)cn4)cnc3C)cc2cn1] |
| 67 | 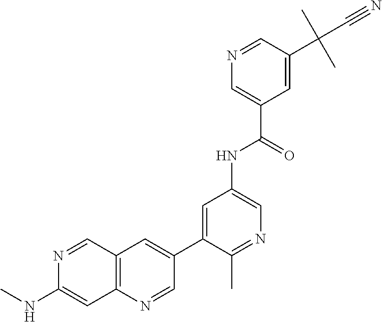 [SMILES: CNc1cc2ncc(-c3cc(NC(=O)c4cncc(C(C)(C)C#N)c4)cnc3C)cc2cn1] |
| 68 | 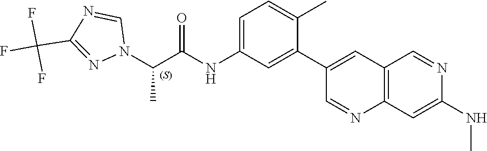 |
| 69 | 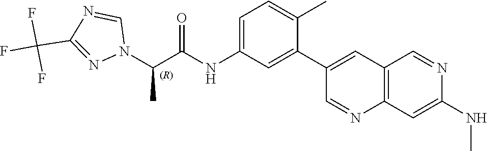 |
| 70 | 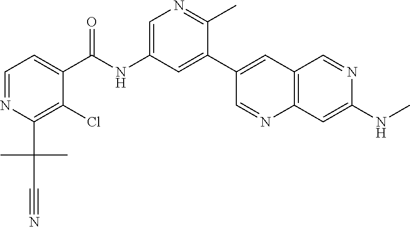 [SMILES: CNc1cc2ncc(-c3cc(NC(=O)c4ccnc(C(C)(C)C#N)c4Cl)cnc3C)cc2cn1] |
| 71 | 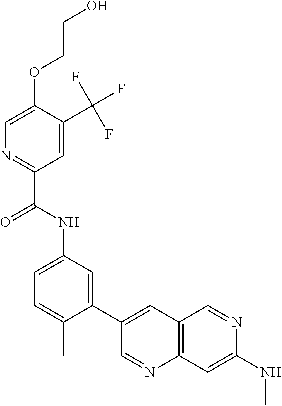 [SMILES: CNc1cc2ncc(-c3cc(NC(=O)c4cc(C(F)(F)F)c(OCCO)cn4)ccc3C)cc2cn1] |
| 72 | 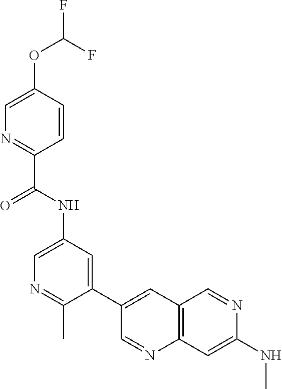 [SMILES: CNc1cc2ncc(-c3cc(NC(=O)c4ccc(OC(F)F)cn4)cnc3C)cc2cn1] |
| 73 | 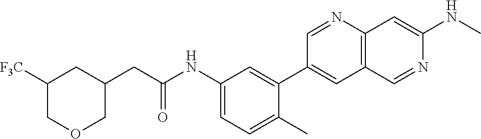 [SMILES: CNc1cc2ncc(-c3cc(NC(=O)CC4COCC(C(F)(F)F)C4)ccc3C)cc2cn1] |
| 74 | 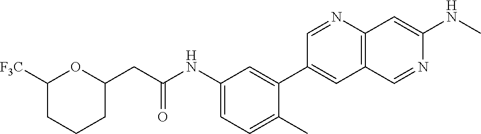 [SMILES: CNc1cc2ncc(-c3cc(NC(=O)CC4CCCC(C(F)(F)F)O4)ccc3C)cc2cn1] |
| 75 | 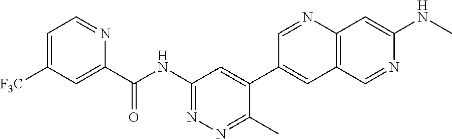 [SMILES: CNc1cc2ncc(-c3cc(NC(=O)c4cc(C(F)(F)F)ccn4)nnc3C)cc2cn1] |
| 76 | 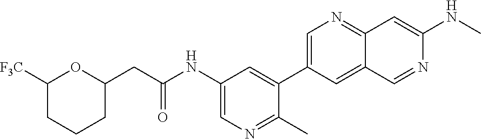 [SMILES: CNc1cc2ncc(-c3cc(NC(=O)CC4CCCC(C(F)(F)F)O4)cnc3C)cc2cn1] |
| 77 | 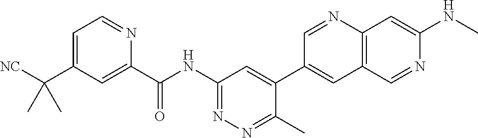 [SMILES: CNc1cc2ncc(-c3cc(NC(=O)c4cc(C(C)(C)C#N)ccn4)nnc3C)cc2cn1] |
| 78 | 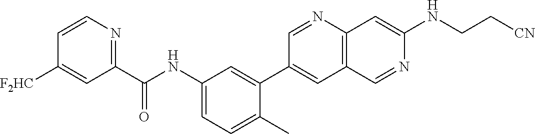 [SMILES: Cc1ccc(NC(=O)c2cc(C(F)F)ccn2)cc1-c1cnc2cc(NCCC#N)ncc2c1] |
| 79 | 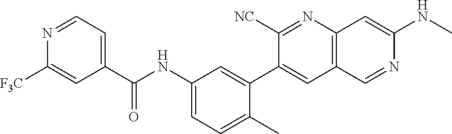 [SMILES: CNc1cc2nc(C#N)c(-c3cc(NC(=O)c4ccnc(C(F)(F)F)c4)ccc3C)cc2cn1] |
| 80 | 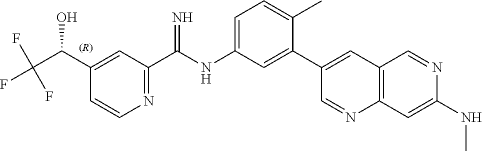 |
| 81 | 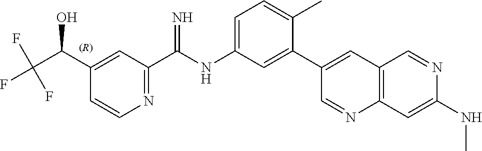 |
| 82 | 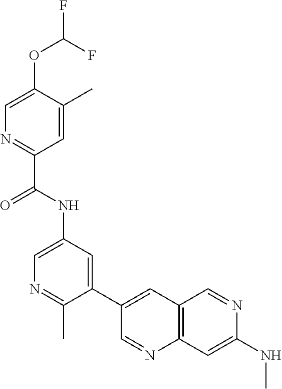 [SMILES: CNc1cc2ncc(-c3cc(NC(=O)c4cc(C)c(OC(F)F)cn4)cnc3C)cc2cn1] |
| 83 | 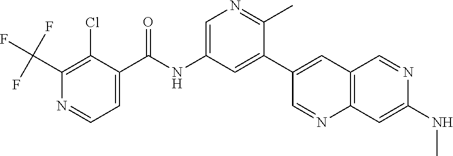 [SMILES: CNc1cc2ncc(-c3cc(NC(=O)c4ccnc(C(F)(F)F)c4Cl)cnc3C)cc2cn1] |
| 84 | 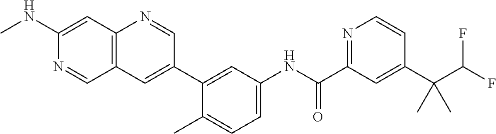 [SMILES: CNc1cc2ncc(-c3cc(NC(=O)c4cc(C(C)(C)C(F)F)ccn4)ccc3C)cc2cn1] |
| 85 | 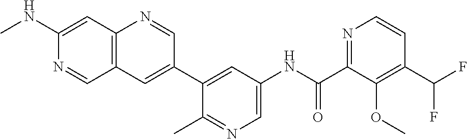 [SMILES: CNc1cc2ncc(-c3cc(NC(=O)c4nccc(C(F)F)c4OC)cnc3C)cc2cn1] |
| 86 | 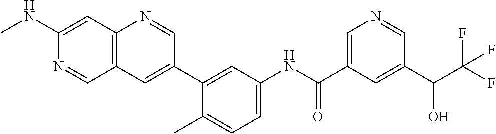 [SMILES: CNc1cc2ncc(-c3cc(NC(=O)c4cncc(C(O)C(F)(F)F)c4)ccc3C)cc2cn1] |
| 87 | 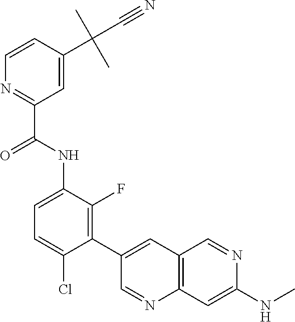 [SMILES: CNc1cc2ncc(-c3c(Cl)ccc(NC(=O)c4cc(C(C)(C)C#N)ccn4)c3F)cc2cn1] |
| 88 | 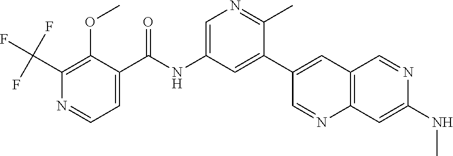 [SMILES: CNc1cc2ncc(-c3cc(NC(=O)c4ccnc(C(F)(F)F)c4OC)cnc3C)cc2cn1] |
| 89 | 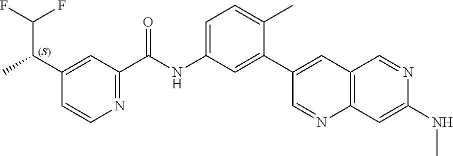 |
| 90 | 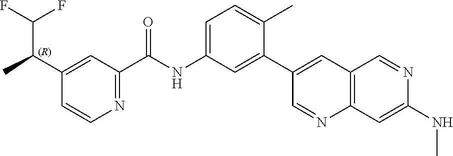 |
| 91 | 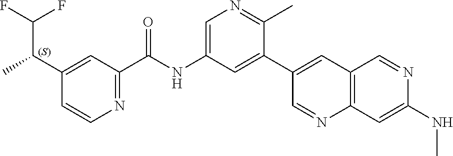 |
| 92 | 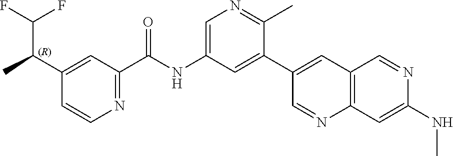 |
| 93 | 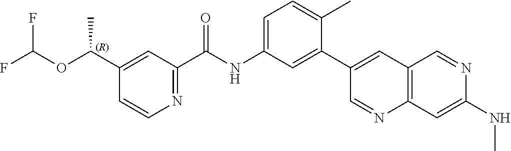 |
| 94 | 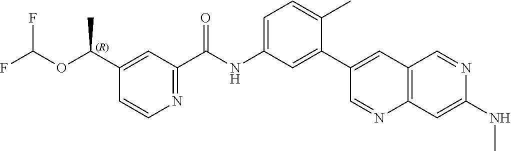 |
| 95 | 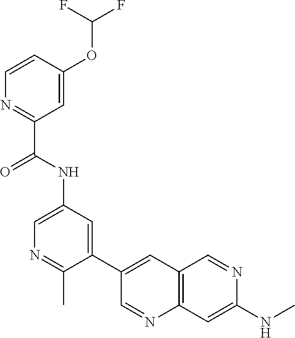 [SMILES: CNc1cc2ncc(-c3cc(NC(=O)c4cc(OC(F)F)ccn4)cnc3C)cc2cn1] |
| 96 | 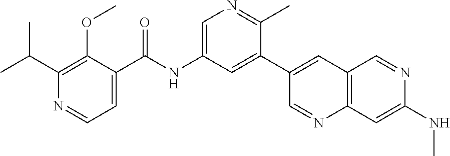 [SMILES: CNc1cc2ncc(-c3cc(NC(=O)c4ccnc(C(C)C)c4OC)cnc3C)cc2cn1] |
| 97 | 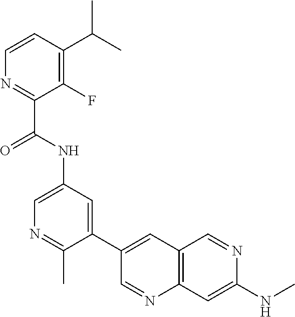 [SMILES: CNc1cc2ncc(-c3cc(NC(=O)c4nccc(C(C)C)c4F)cnc3C)cc2cn1] |
| 98 | 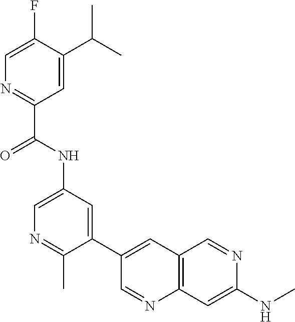 [SMILES: CNc1cc2ncc(-c3cc(NC(=O)c4cc(C(C)C)c(F)cn4)cnc3C)cc2cn1] |
| 99 | 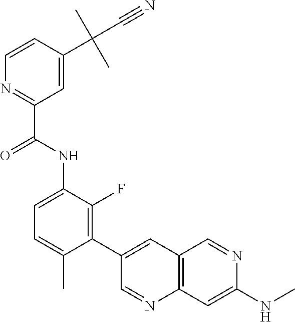 [SMILES: CNc1cc2ncc(-c3c(C)ccc(NC(=O)c4cc(C(C)(C)C#N)ccn4)c3F)cc2cn1] |
| 100 | 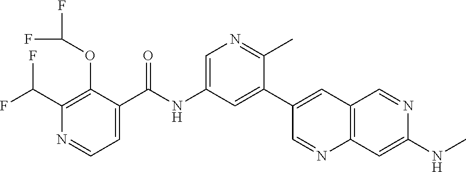 [SMILES: CNc1cc2ncc(-c3cc(NC(=O)c4ccnc(C(F)F)c4OC(F)F)cnc3C)cc2cn1] |
| 101 | 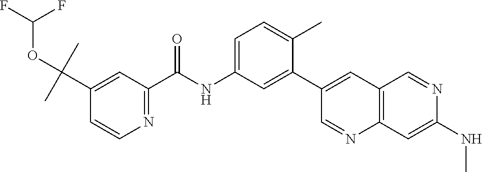 [SMILES: CNc1cc2ncc(-c3cc(NC(=O)c4cc(C(C)(C)OC(F)F)ccn4)ccc3C)cc2cn1] |
| 102 | 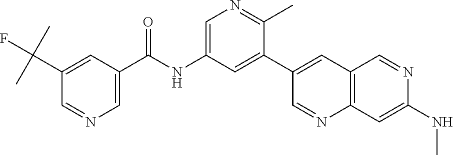 [SMILES: CNc1cc2ncc(-c3cc(NC(=O)c4cncc(C(C)(C)F)c4)cnc3C)cc2cn1] |
| 103 | 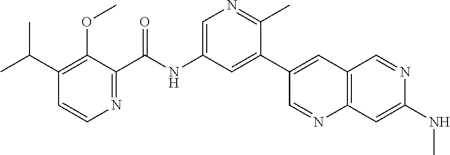 [SMILES: CNc1cc2ncc(-c3cc(NC(=O)c4nccc(C(C)C)c4OC)cnc3C)cc2cn1] |
| 104 | 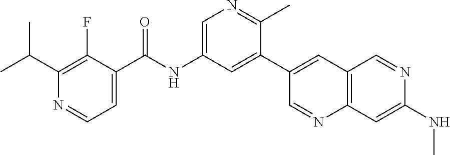 [SMILES: CNc1cc2ncc(-c3cc(NC(=O)c4ccnc(C(C)C)c4F)cnc3C)cc2cn1] |
| 105 | 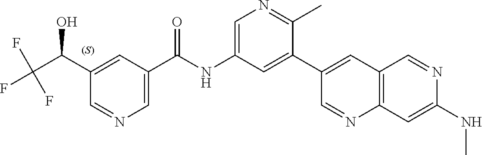 |
| 106 | 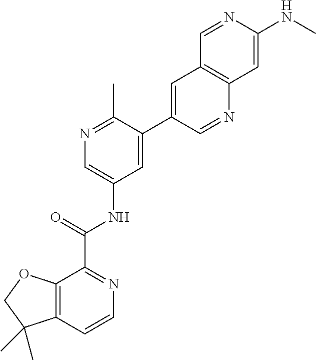 |
| 107 | 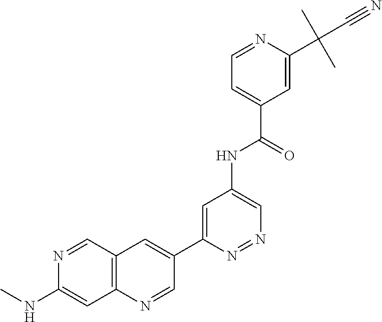 [SMILES: CNc1cc2ncc(-c3cc(NC(=O)c4ccnc(C(C)(C)C#N)c4)cnn3)cc2cn1] |
| 108 | 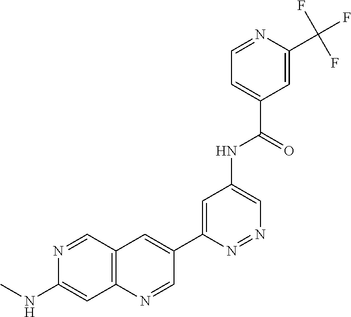 [SMILES: CNc1cc2ncc(-c3cc(NC(=O)c4ccnc(C(F)(F)F)c4)cnn3)cc2cn1] |
| 109 | 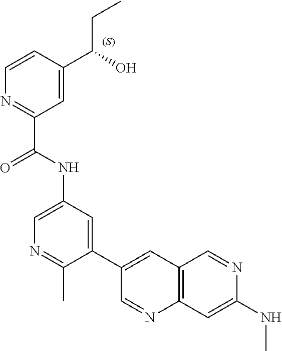 |
| 110 | 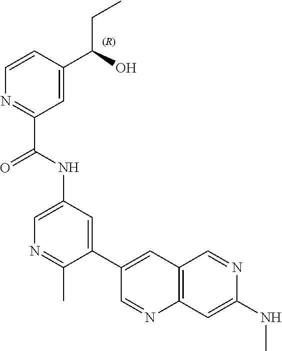 |
| 111 | 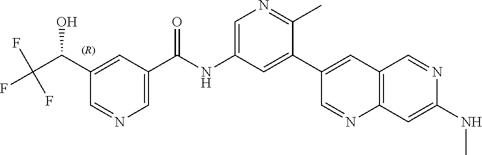 |
| 112 | 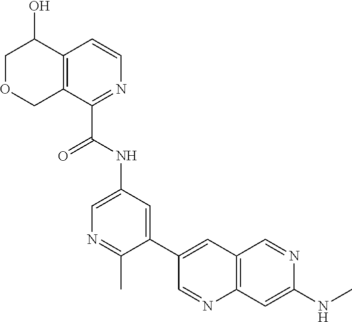 [SMILES: CNc1cc2ncc(-c3cc(NC(=O)c4nccc5c4COCC5O)cnc3C)cc2cn1] |
| 113 | 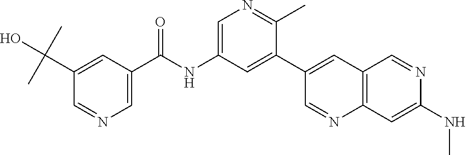 [SMILES: CNc1cc2ncc(-c3cc(NC(=O)c4cncc(C(C)(C)O)c4)cnc3C)cc2cn1] |
| 114 | 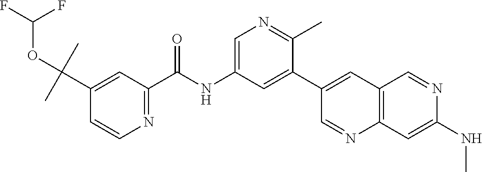 [SMILES: CNc1cc2ncc(-c3cc(NC(=O)c4cc(C(C)(C)OC(F)F)ccn4)cnc3C)cc2cn1] |
| 115 | 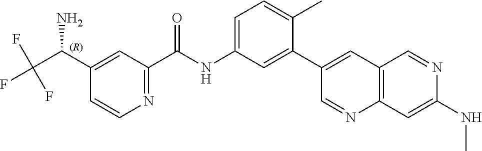 |
| 116 | 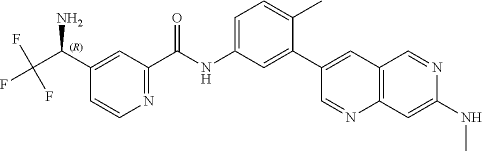 |
| 117 | 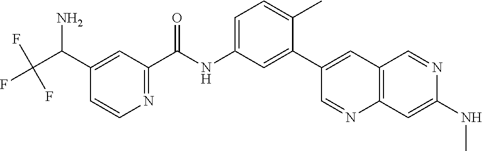 [SMILES: CNc1cc2ncc(-c3cc(NC(=O)c4cc(C(N)C(F)(F)F)ccn4)ccc3C)cc2cn1] |
| 118 | 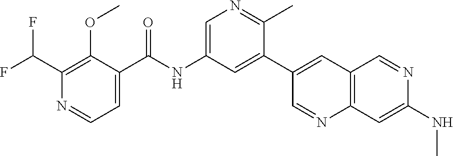 [SMILES: CNc1cc2ncc(-c3cc(NC(=O)c4ccnc(C(F)F)c4OC)cnc3C)cc2cn1] |
| 119 | 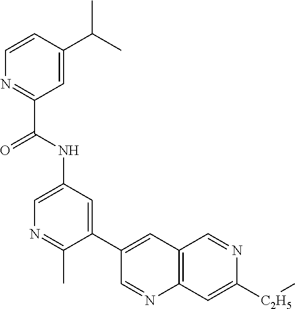 |
| 120 | 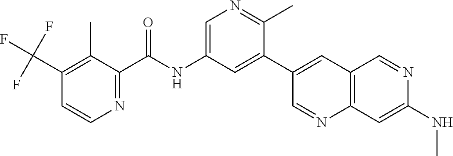 [SMILES: CNc1cc2ncc(-c3cc(NC(=O)c4nccc(C(F)(F)F)c4C)cnc3C)cc2cn1] |
| 121 | 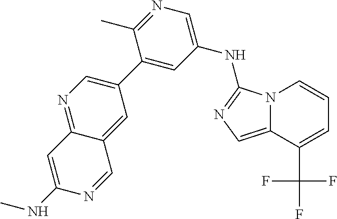 [SMILES: CNc1cc2ncc(-c3cc(Nc4ncc5c(C(F)(F)F)cccn45)cnc3C)cc2cn1] |
| 122 | 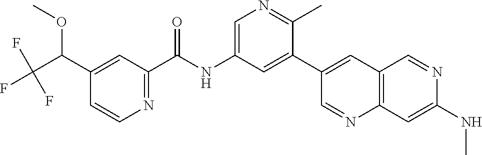 [SMILES: CNc1cc2ncc(-c3cc(NC(=O)c4cc(C(OC)C(F)(F)F)ccn4)cnc3C)cc2cn1] |
| 123 | 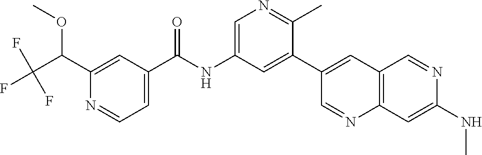 [SMILES: CNc1cc2ncc(-c3cc(NC(=O)c4ccnc(C(OC)C(F)(F)F)c4)cnc3C)cc2cn1] |
| 124 | 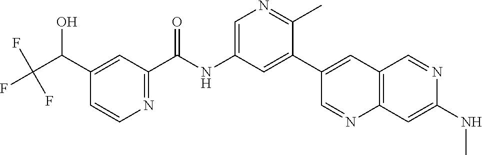 [SMILES: CNc1cc2ncc(-c3cc(NC(=O)c4cc(C(O)C(F)(F)F)ccn4)cnc3C)cc2cn1] |
| 125 | 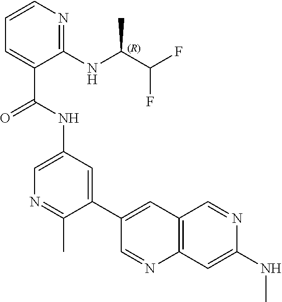 |
| 126 | 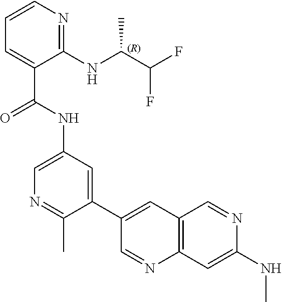 |
| 127 | 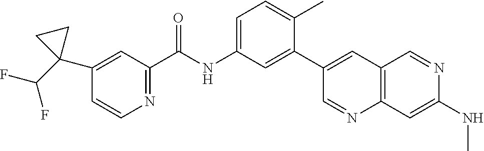 [SMILES: CNc1cc2ncc(-c3cc(NC(=O)c4cc(C5(C(F)F)CC5)ccn4)ccc3C)cc2cn1] |
| 128 | 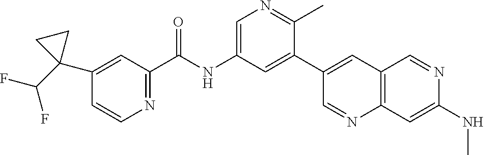 [SMILES: CNc1cc2ncc(-c3cc(NC(=O)c4cc(C5(C(F)F)CC5)ccn4)cnc3C)cc2cn1] |
| 129 | 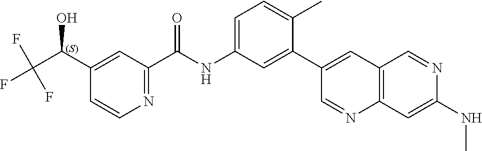 |
| 130 | 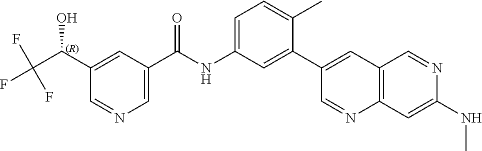 |
| 131 | 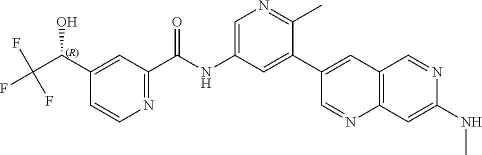 |
| 132 | 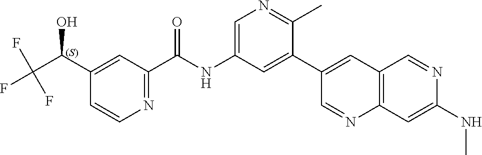 |
| 133 | 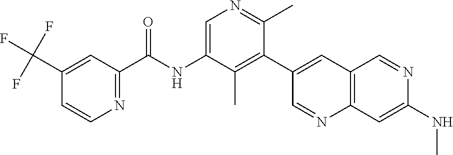 [SMILES: CNc1cc2ncc(-c3c(C)ncc(NC(=O)c4cc(C(F)(F)F)ccn4)c3C)cc2cn1] |
| 134 | 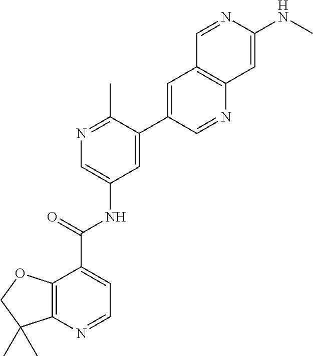 [SMILES: CNc1cc2ncc(-c3cc(NC(=O)c4ccnc5c4OCC5(C)C)cnc3C)cc2cn1] |
| 135 | 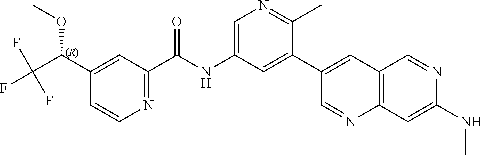 |
| 136 | 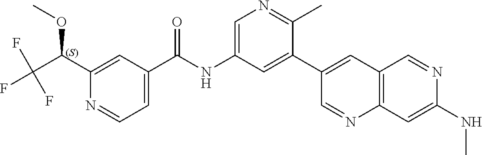 |
| 137 | 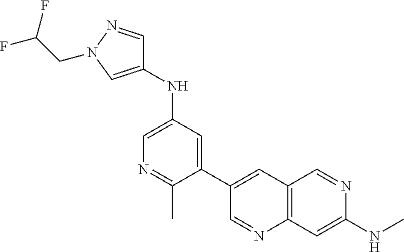 [SMILES: CNc1cc2ncc(-c3cc(Nc4cnn(CC(F)F)c4)cnc3C)cc2cn1] |
| 138 | 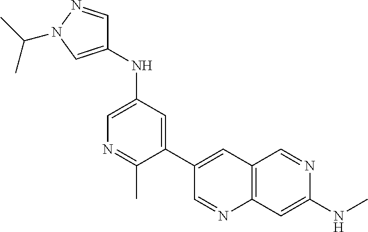 [SMILES: CNc1cc2ncc(-c3cc(Nc4cnn(C(C)C)c4)cnc3C)cc2cn1] |
| 139 | 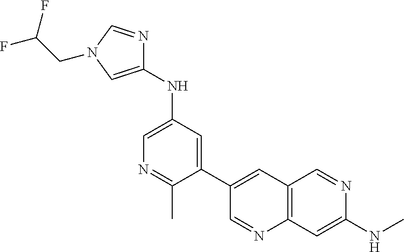 [SMILES: CNc1cc2ncc(-c3cc(Nc4cn(CC(F)F)cn4)cnc3C)cc2cn1] |
| 140 | 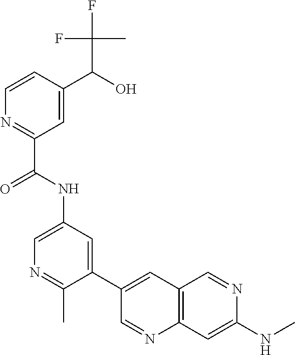 [SMILES: CNc1cc2ncc(-c3cc(NC(=O)c4cc(C(O)C(C)(F)F)ccn4)cnc3C)cc2cn1] |
| 141 | 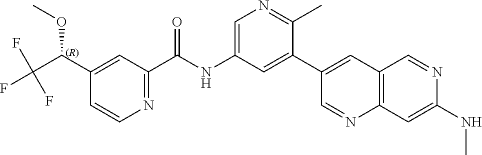 |
| 142 | 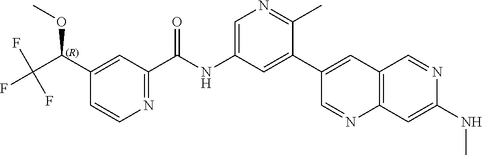 |
| 143 | 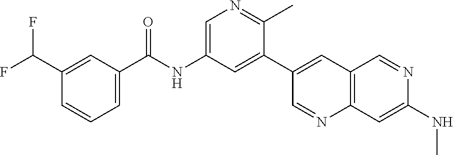 [SMILES: CNc1cc2ncc(-c3cc(NC(=O)c4cccc(C(F)F)c4)cnc3C)cc2cn1] |
| 144 | 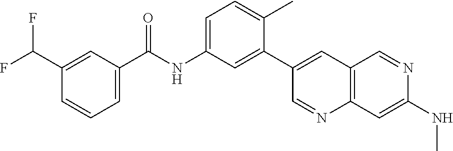 [SMILES: CNc1cc2ncc(-c3cc(NC(=O)c4cccc(C(F)F)c4)ccc3C)cc2cn1] |
| 145 | 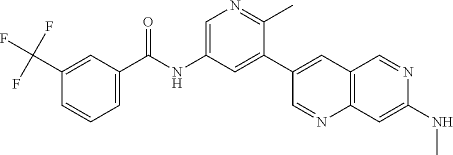 [SMILES: CNc1cc2ncc(-c3cc(NC(=O)c4cccc(C(F)(F)F)c4)cnc3C)cc2cn1] |
| 146 | 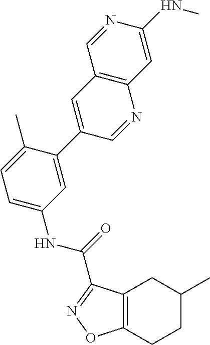 [SMILES: CNc1cc2ncc(-c3cc(NC(=O)c4noc5c4CC(C)CC5)ccc3C)cc2cn1] |
| 147 | 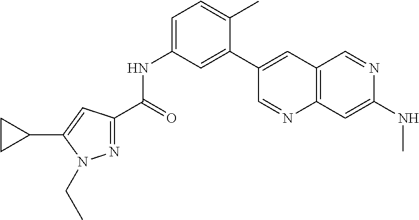 [SMILES: CCn1nc(C(=O)Nc2ccc(C)c(-c3cnc4cc(NC)ncc4c3)c2)cc1C1CC1] |
| 148 | 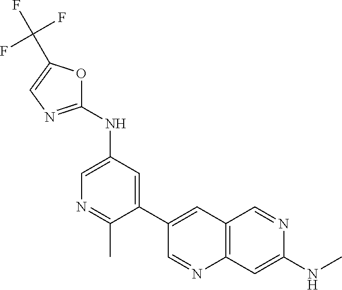 [SMILES: CNc1cc2ncc(-c3cc(Nc4ncc(C(F)(F)F)o4)cnc3C)cc2cn1] |
| 149 | 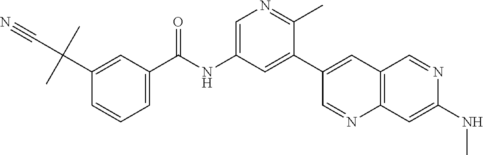 [SMILES: CNc1cc2ncc(-c3cc(NC(=O)c4cccc(C(C)(C)C#N)c4)cnc3C)cc2cn1] |
| 150 | 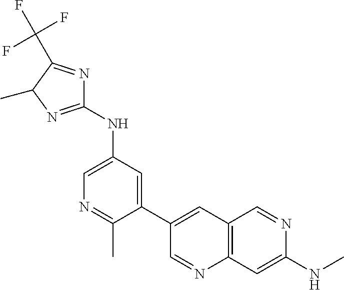 |
| 151 | 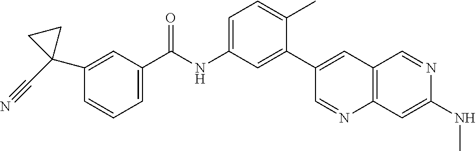 [SMILES: CNc1cc2ncc(-c3cc(NC(=O)c4cccc(C5(C#N)CC5)c4)ccc3C)cc2cn1] |
| 152 | 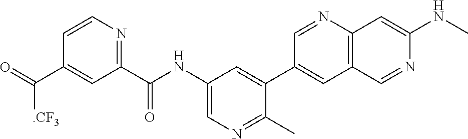 |
| 153 | 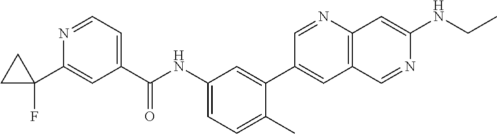 [SMILES: CCNc1cc2ncc(-c3cc(NC(=O)c4ccnc(C5(F)CC5)c4)ccc3C)cc2cn1] |
| 154 | 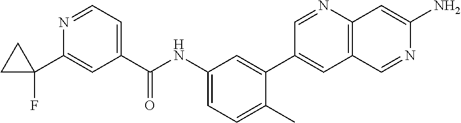 [SMILES: Cc1ccc(NC(=O)c2ccnc(C3(F)CC3)c2)cc1-c1cnc2cc(N)ncc2c1] |
| 155 | 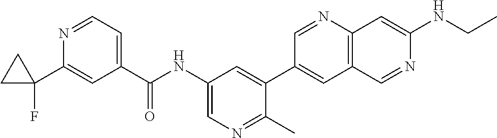 [SMILES: CCNc1cc2ncc(-c3cc(NC(=O)c4ccnc(C5(F)CC5)c4)cnc3C)cc2cn1] |
| 156 | 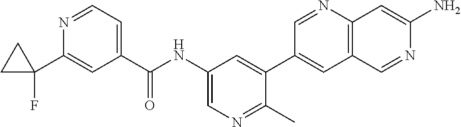 [SMILES: Cc1ncc(NC(=O)c2ccnc(C3(F)CC3)c2)cc1-c1cnc2cc(N)ncc2c1] |
| 157 | 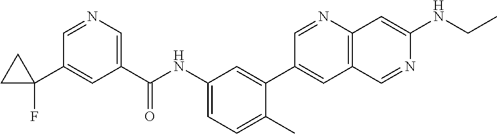 [SMILES: CCNc1cc2ncc(-c3cc(NC(=O)c4cncc(C5(F)CC5)c4)ccc3C)cc2cn1] |
| 158 | 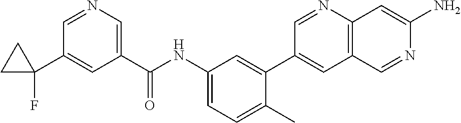 [SMILES: Cc1ccc(NC(=O)c2cncc(C3(F)CC3)c2)cc1-c1cnc2cc(N)ncc2c1] |
| 159 | 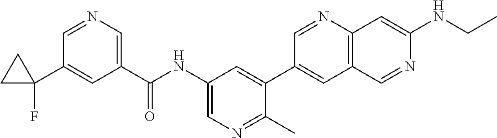 [SMILES: CCNc1cc2ncc(-c3cc(NC(=O)c4cncc(C5(F)CC5)c4)cnc3C)cc2cn1] |
| 160 | 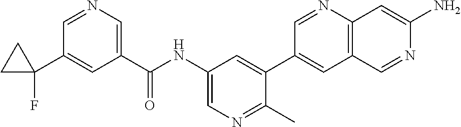 [SMILES: Cc1ncc(NC(=O)c2cncc(C3(F)CC3)c2)cc1-c1cnc2cc(N)ncc2c1] |
| 161 | 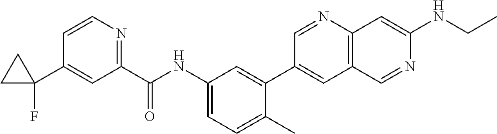 [SMILES: CCNc1cc2ncc(-c3cc(NC(=O)c4cc(C5(F)CC5)ccn4)ccc3C)cc2cn1] |
| 162 | 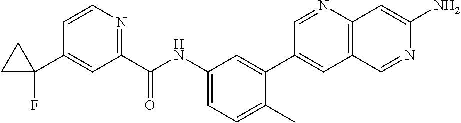 [SMILES: Cc1ccc(NC(=O)c2cc(C3(F)CC3)ccn2)cc1-c1cnc2cc(N)ncc2c1] |
| 163 | 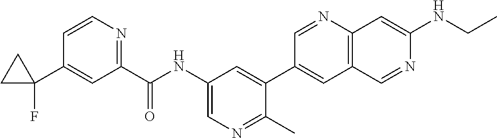 [SMILES: CCNc1cc2ncc(-c3cc(NC(=O)c4cc(C5(F)CC5)ccn4)cnc3C)cc2cn1] |
| 164 | 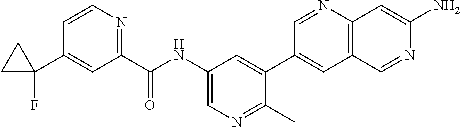 [SMILES: Cc1ncc(NC(=O)c2cc(C3(F)CC3)ccn2)cc1-c1cnc2cc(N)ncc2c1] |
| 165 | 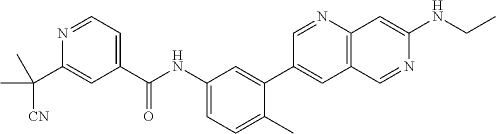 [SMILES: CCNc1cc2ncc(-c3cc(NC(=O)c4ccnc(C(C)(C)C#N)c4)ccc3C)cc2cn1] |
| 166 | 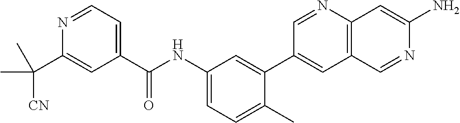 [SMILES: Cc1ccc(NC(=O)c2ccnc(C(C)(C)C#N)c2)cc1-c1cnc2cc(N)ncc2c1] |
| 167 | 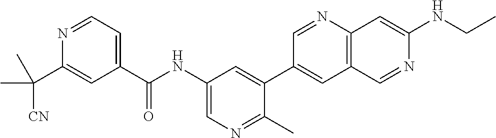 [SMILES: CCNc1cc2ncc(-c3cc(NC(=O)c4ccnc(C(C)(C)C#N)c4)cnc3C)cc2cn1] |
| 168 | 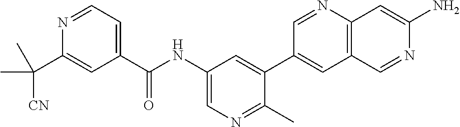 [SMILES: Cc1ncc(NC(=O)c2ccnc(C(C)(C)C#N)c2)cc1-c1cnc2cc(N)ncc2c1] |
| 169 | 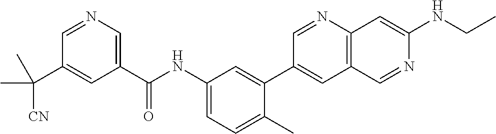 [SMILES: CCNc1cc2ncc(-c3cc(NC(=O)c4cncc(C(C)(C)C#N)c4)ccc3C)cc2cn1] |
| 170 | 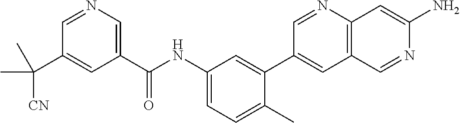 [SMILES: Cc1ccc(NC(=O)c2cncc(C(C)(C)C#N)c2)cc1-c1cnc2cc(N)ncc2c1] |
| 171 | 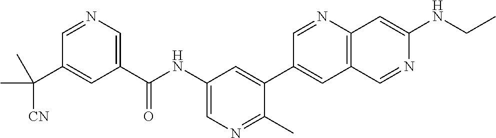 [SMILES: CCNc1cc2ncc(-c3cc(NC(=O)c4cncc(C(C)(C)C#N)c4)cnc3C)cc2cn1] |
| 172 | 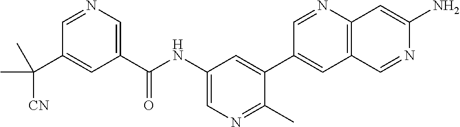 [SMILES: Cc1ncc(NC(=O)c2cncc(C(C)(C)C#N)c2)cc1-c1cnc2cc(N)ncc2c1] |
| 173 | 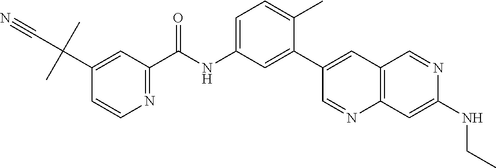 [SMILES: CCNc1cc2ncc(-c3cc(NC(=O)c4cc(C(C)(C)C#N)ccn4)ccc3C)cc2cn1] |
| 174 | 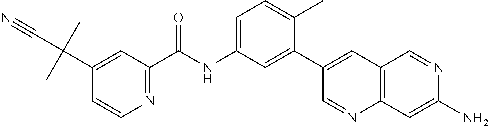 [SMILES: Cc1ccc(NC(=O)c2cc(C(C)(C)C#N)ccn2)cc1-c1cnc2cc(N)ncc2c1] |
| 175 | 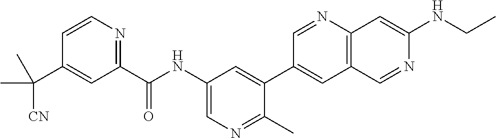 [SMILES: CCNc1cc2ncc(-c3cc(NC(=O)c4cc(C(C)(C)C#N)ccn4)cnc3C)cc2cn1] |
| 176 | 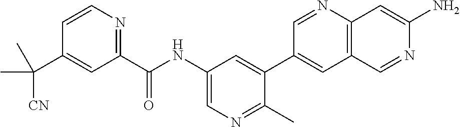 [SMILES: Cc1ncc(NC(=O)c2cc(C(C)(C)C#N)ccn2)cc1-c1cnc2cc(N)ncc2c1] |
| 177 | 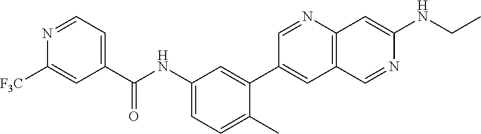 [SMILES: CCNc1cc2ncc(-c3cc(NC(=O)c4ccnc(C(F)(F)F)c4)ccc3C)cc2cn1] |
| 178 | 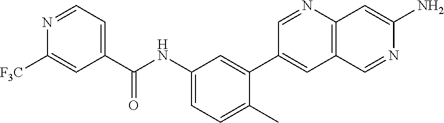 [SMILES: Cc1ccc(NC(=O)c2ccnc(C(F)(F)F)c2)cc1-c1cnc2cc(N)ncc2c1] |
| 179 | 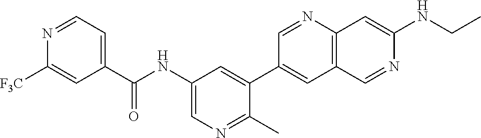 [SMILES: CCNc1cc2ncc(-c3cc(NC(=O)c4ccnc(C(F)(F)F)c4)cnc3C)cc2cn1] |
| 180 | 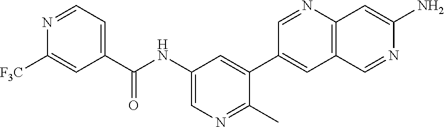 [SMILES: Cc1ncc(NC(=O)c2ccnc(C(F)(F)F)c2)cc1-c1cnc2cc(N)ncc2c1] |
| 181 | 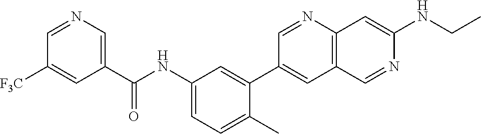 [SMILES: CCNc1cc2ncc(-c3cc(NC(=O)c4cncc(C(F)(F)F)c4)ccc3C)cc2cn1] |
| 182 | 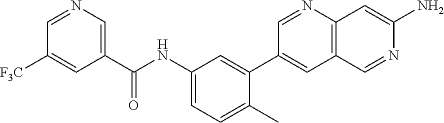 [SMILES: Cc1ccc(NC(=O)c2cncc(C(F)(F)F)c2)cc1-c1cnc2cc(N)ncc2c1] |
| 183 | 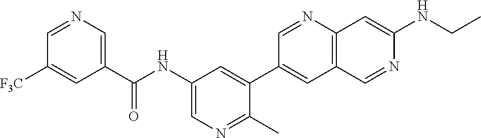 [SMILES: CCNc1cc2ncc(-c3cc(NC(=O)c4cncc(C(F)(F)F)c4)cnc3C)cc2cn1] |
| 184 | 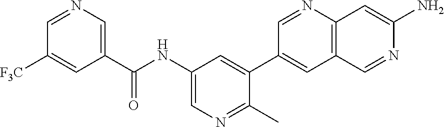 [SMILES: Cc1ncc(NC(=O)c2cncc(C(F)(F)F)c2)cc1-c1cnc2cc(N)ncc2c1] |
| 185 | 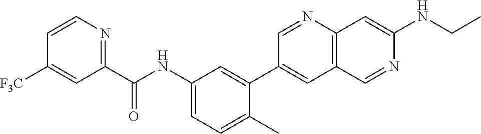 [SMILES: CCNc1cc2ncc(-c3cc(NC(=O)c4cc(C(F)(F)F)ccn4)ccc3C)cc2cn1] |
| 186 | 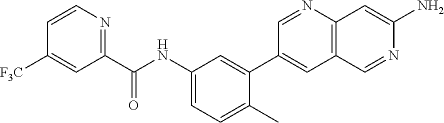 [SMILES: Cc1ccc(NC(=O)c2cc(C(F)(F)F)ccn2)cc1-c1cnc2cc(N)ncc2c1] |
| 187 | 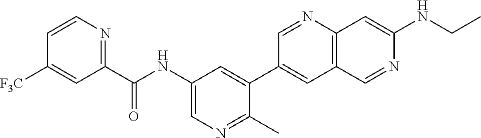 [SMILES: CCNc1cc2ncc(-c3cc(NC(=O)c4cc(C(F)(F)F)ccn4)cnc3C)cc2cn1] |
| 188 | 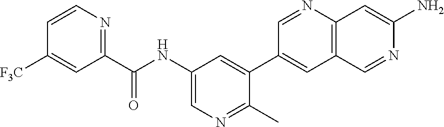 [SMILES: Cc1ncc(NC(=O)c2cc(C(F)(F)F)ccn2)cc1-c1cnc2cc(N)ncc2c1] |

TABLE II

| Compound | |
|---|---|
| No. | Structure |
| 189 | 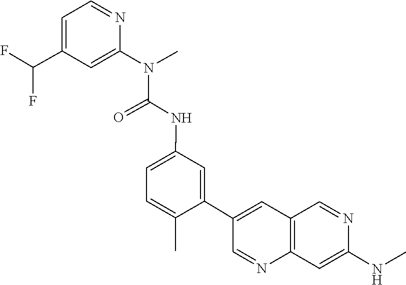 [SMILES: CNc1cc2ncc(-c3cc(NC(=O)N(C)c4cc(C(F)F)ccn4)ccc3C)cc2cn1] |
| 190 | 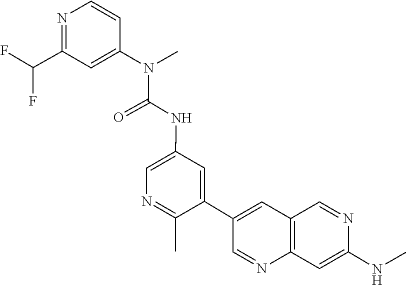 [SMILES: CNc1cc2ncc(-c3cc(NC(=O)N(C)c4ccnc(C(F)F)c4)cnc3C)cc2cn1] |
| 191 | 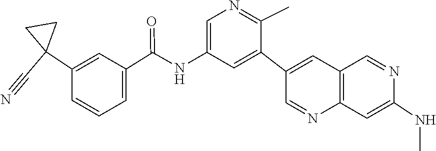 [SMILES: CNc1cc2ncc(-c3cc(NC(=O)c4cccc(C5(C#N)CC5)c4)cnc3C)cc2cn1] |
| 192 | 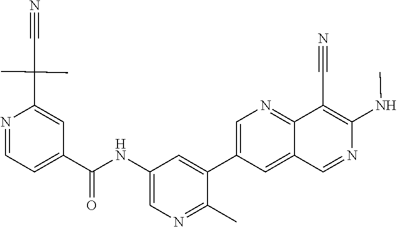 [SMILES: CNc1ncc2cc(-c3cc(NC(=O)c4ccnc(C(C)(C)C#N)c4)cnc3C)cnc2c1C#N] |
| 193 | 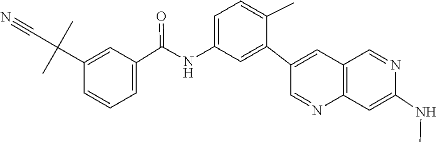 [SMILES: CNc1cc2ncc(-c3cc(NC(=O)c4cccc(C(C)(C)C#N)c4)ccc3C)cc2cn1] |
| 194 | 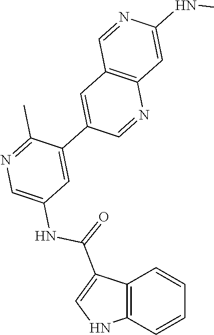 [SMILES: CNc1cc2ncc(-c3cc(NC(=O)c4c[nH]c5ccccc45)cnc3C)cc2cn1] |
| 195 | 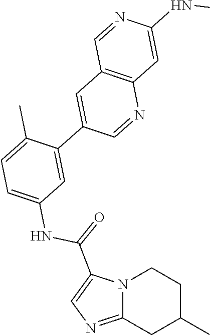 [SMILES: CNc1cc2ncc(-c3cc(NC(=O)c4cnc5n4CCC(C)C5)ccc3C)cc2cn1] |
| 196 | 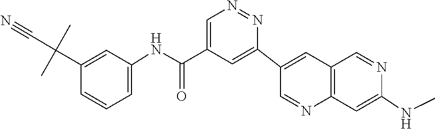 [SMILES: CNc1cc2ncc(-c3cc(C(=O)Nc4cccc(C(C)(C)C#N)c4)cnn3)cc2cn1] |
| 197 | 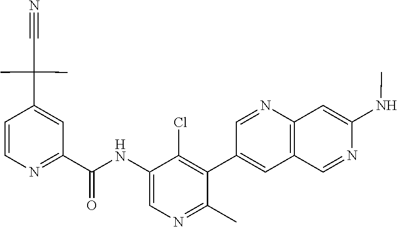 [SMILES: CNc1cc2ncc(-c3c(C)ncc(NC(=O)c4cc(C(C)(C)C#N)ccn4)c3Cl)cc2cn1] |
| 198 | 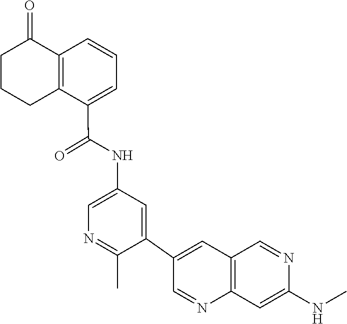 [SMILES: CNc1cc2ncc(-c3cc(NC(=O)c4cccc5c4CCCC5=O)cnc3C)cc2cn1] |
| 199 | 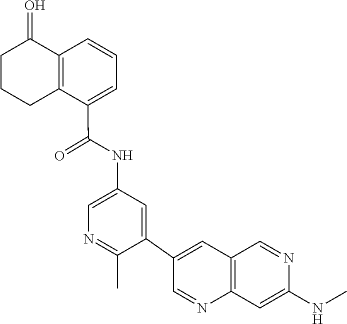 [SMILES: CNc1cc2ncc(-c3cc(NC(=O)c4cccc5c4CCCC5=O)cnc3C)cc2cn1] |
| 200 | 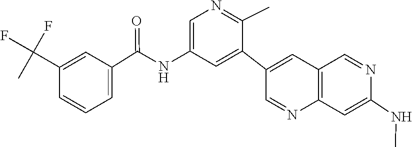 [SMILES: CNc1cc2ncc(-c3cc(NC(=O)c4cccc(C(C)(F)F)c4)cnc3C)cc2cn1] |
| 201 | 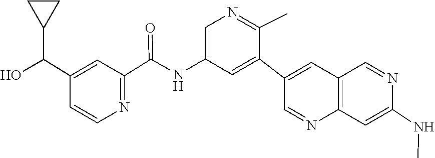 [SMILES: CNc1cc2ncc(-c3cc(NC(=O)c4cc(C(O)C5CC5)ccn4)cnc3C)cc2cn1] |
| 202 | 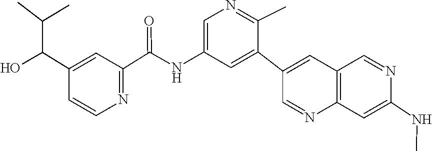 [SMILES: CNc1cc2ncc(-c3cc(NC(=O)c4cc(C(O)C(C)C)ccn4)cnc3C)cc2cn1] |
| 203 | 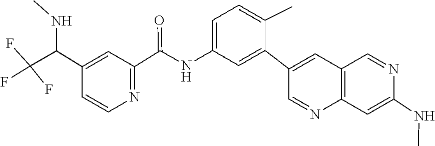 [SMILES: CNc1cc2ncc(-c3cc(NC(=O)c4cc(C(NC)C(F)(F)F)ccn4)ccc3C)cc2cn1] |
| 204 | 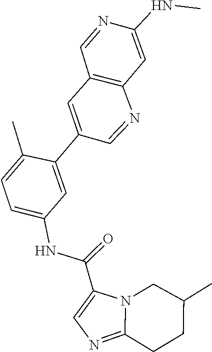 [SMILES: CNc1cc2ncc(-c3cc(NC(=O)c4cnc5n4CC(C)CC5)ccc3C)cc2cn1] |
| 205 | 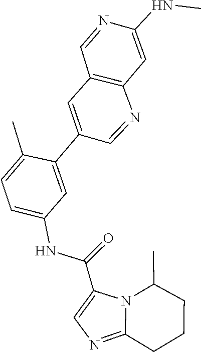 [SMILES: CNc1cc2ncc(-c3cc(NC(=O)c4cnc5n4C(C)CCC5)ccc3C)cc2cn1] |
| 206 | 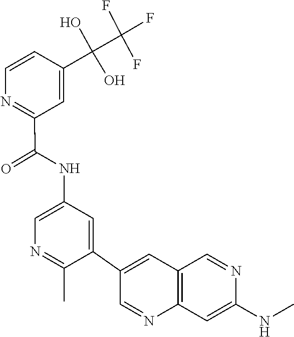 [SMILES: CNc1cc2ncc(-c3cc(NC(=O)c4cc(C(O)(O)C(F)(F)F)ccn4)cnc3C)cc2cn1] |
| 207 | 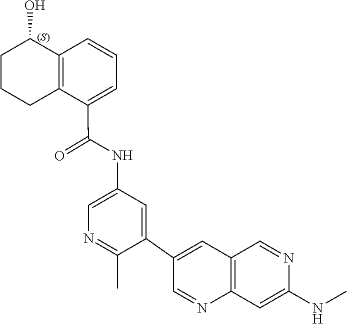 |
| 208 | 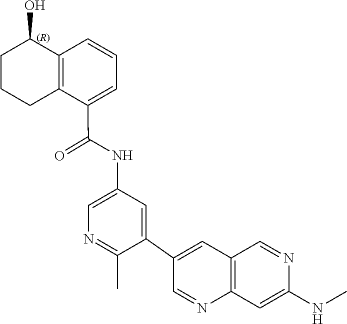 |
| 209 | 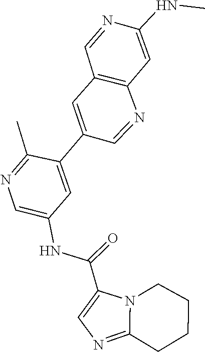 [SMILES: CNc1cc2ncc(-c3cc(NC(=O)c4cnc5n4CCCC5)cnc3C)cc2cn1] |
| 210 | 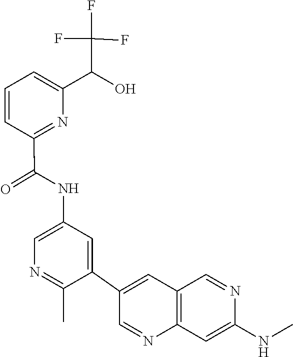 [SMILES: CNc1cc2ncc(-c3cc(NC(=O)c4cccc(C(O)C(F)(F)F)n4)cnc3C)cc2cn1] |
| 211 | 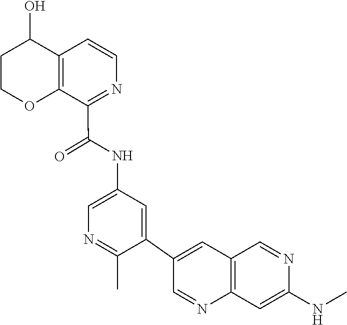 [SMILES: CNc1cc2ncc(-c3cc(NC(=O)c4nccc5c4OCCC5O)cnc3C)cc2cn1] |
| 212 | 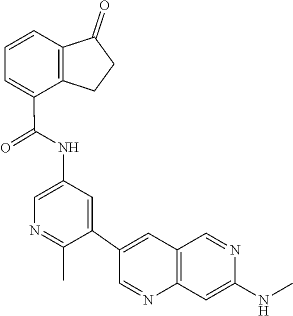 [SMILES: CNc1cc2ncc(-c3cc(NC(=O)c4cccc5c4CCC5=O)cnc3C)cc2cn1] |
| 213 | 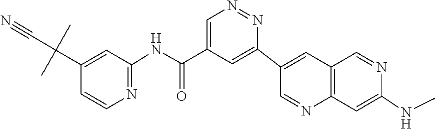 [SMILES: CNc1cc2ncc(-c3cc(C(=O)Nc4cc(C(C)(C)C#N)ccn4)cnn3)cc2cn1] |
| 214 | 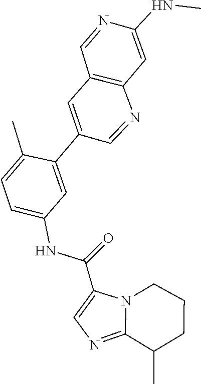 [SMILES: CNc1cc2ncc(-c3cc(NC(=O)c4cnc5n4CCCC5C)ccc3C)cc2cn1] |
| 215 | 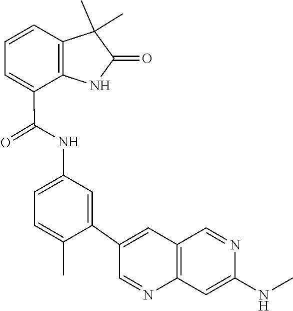 [SMILES: CNc1cc2ncc(-c3cc(NC(=O)c4cccc5c4NC(=O)C5(C)C)ccc3C)cc2cn1] |
| 216 | 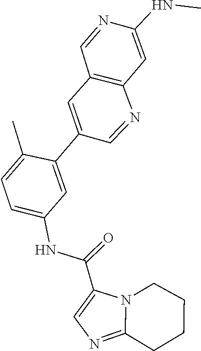 [SMILES: CNc1cc2ncc(-c3cc(NC(=O)c4cnc5n4CCCC5)ccc3C)cc2cn1] |
| 217 | 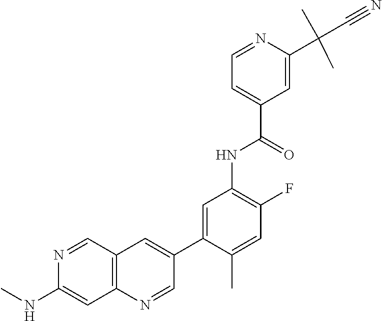 [SMILES: CNc1cc2ncc(-c3cc(NC(=O)c4ccnc(C(C)(C)C#N)c4)c(F)cc3C)cc2cn1] |
| 218 | 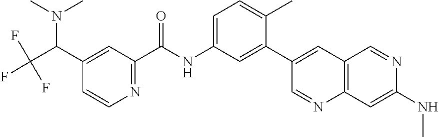 [SMILES: CNc1cc2ncc(-c3cc(NC(=O)c4cc(C(N(C)C)C(F)(F)F)ccn4)ccc3C)cc2cn1] |
| 219 | 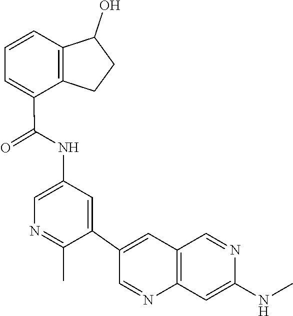 [SMILES: CNc1cc2ncc(-c3cc(NC(=O)c4cccc5c4CCC5O)cnc3C)cc2cn1] |
| 220 | 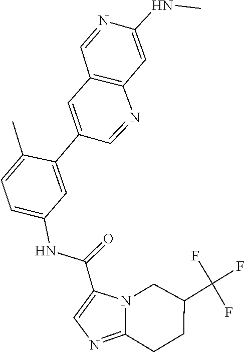 [SMILES: CNc1cc2ncc(-c3cc(NC(=O)c4cnc5n4CC(C(F)(F)F)CC5)ccc3C)cc2cn1] |
| 221 | 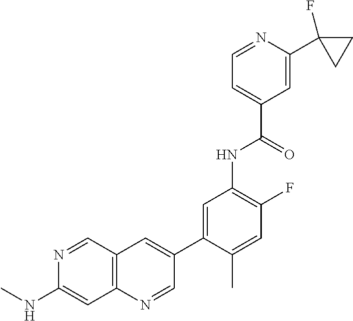 [SMILES: CNc1cc2ncc(-c3cc(NC(=O)c4ccnc(C5(F)CC5)c4)c(F)cc3C)cc2cn1] |
| 222 | 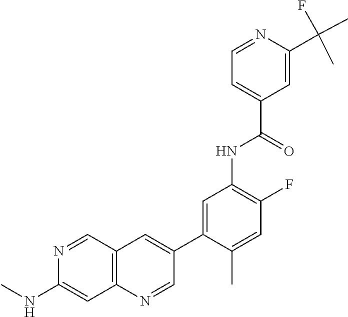 [SMILES: CNc1cc2ncc(-c3cc(NC(=O)c4ccnc(C(C)(C)F)c4)c(F)cc3C)cc2cn1] |
| 223 | 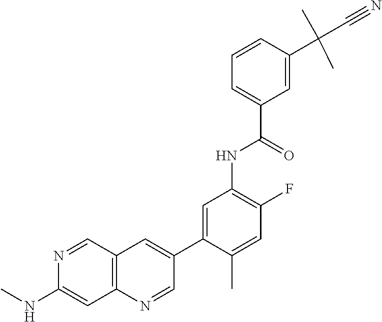 [SMILES: CNc1cc2ncc(-c3cc(NC(=O)c4cccc(C(C)(C)C#N)c4)c(F)cc3C)cc2cn1] |
| 224 | 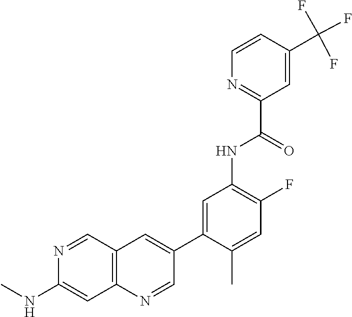 [SMILES: CNc1cc2ncc(-c3cc(NC(=O)c4cc(C(F)(F)F)ccn4)c(F)cc3C)cc2cn1] |
| 225 | 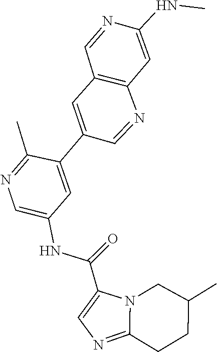 [SMILES: CNc1cc2ncc(-c3cc(NC(=O)c4cnc5n4CC(C)CC5)cnc3C)cc2cn1] |
| 226 | 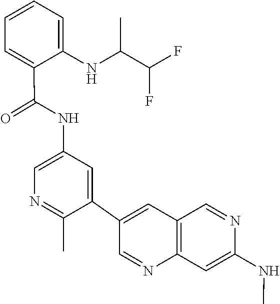 [SMILES: CNc1cc2ncc(-c3cc(NC(=O)c4ccccc4NC(C)C(F)F)cnc3C)cc2cn1] |
| 227 | 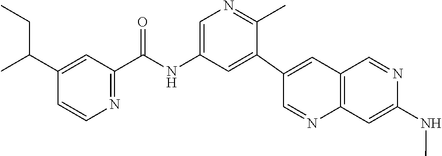 [SMILES: CCC(C)c1ccnc(C(=O)Nc2cnc(C)c(-c3cnc4cc(NC)ncc4c3)c2)c1] |
| 228 | 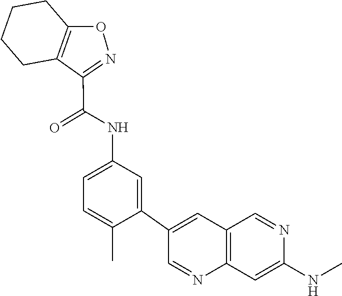 [SMILES: CNc1cc2ncc(-c3cc(NC(=O)c4noc5c4CCCC5)ccc3C)cc2cn1] |
| 229 | 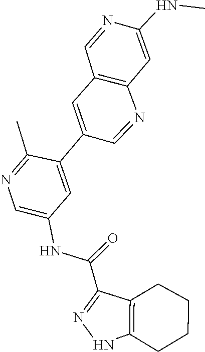 [SMILES: CNc1cc2ncc(-c3cc(NC(=O)c4n[nH]c5c4CCCC5)cnc3C)cc2cn1] |
| 230 | 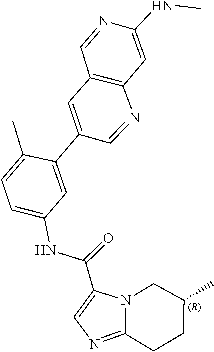 |
| 231 | 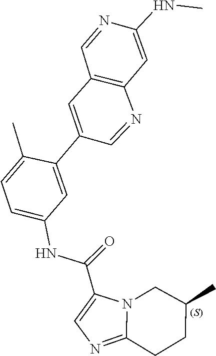 |
| 232 | 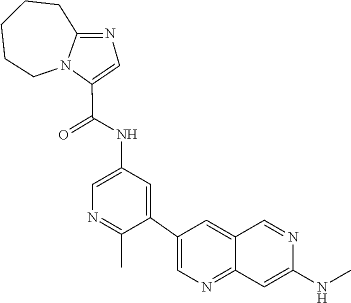 [SMILES: CNc1cc2ncc(-c3cc(NC(=O)c4cnc5n4CCCCC5)cnc3C)cc2cn1] |
| 233 | 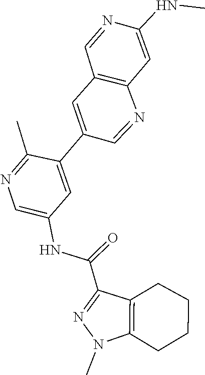 [SMILES: CNc1cc2ncc(-c3cc(NC(=O)c4nn(C)c5c4CCCC5)cnc3C)cc2cn1] |
| 234 | 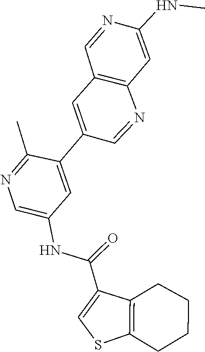 [SMILES: CNc1cc2ncc(-c3cc(NC(=O)c4csc5c4CCCC5)cnc3C)cc2cn1] |
| 235 | 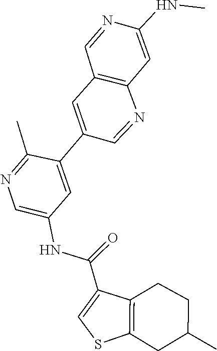 [SMILES: CNc1cc2ncc(-c3cc(NC(=O)c4csc5c4CCC(C)C5)cnc3C)cc2cn1] |
| 236 | 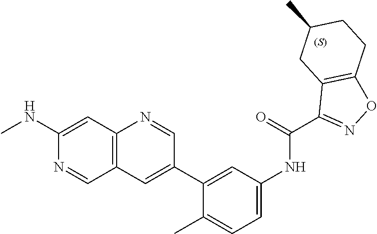 |
| 237 | 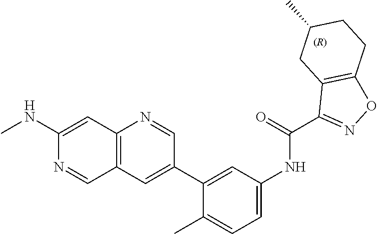 |
| 238 | 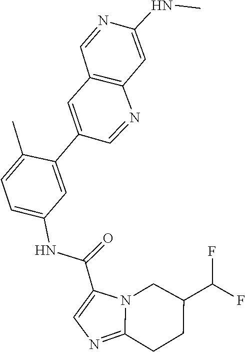 [SMILES: CNc1cc2ncc(-c3cc(NC(=O)c4cnc5n4CC(C(F)F)CC5)ccc3C)cc2cn1] |
| 239 | 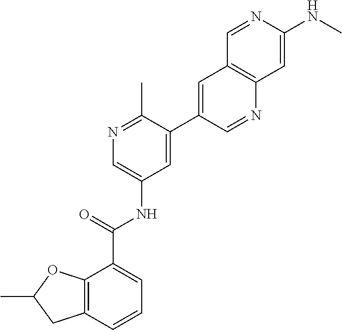 |
| 240 | 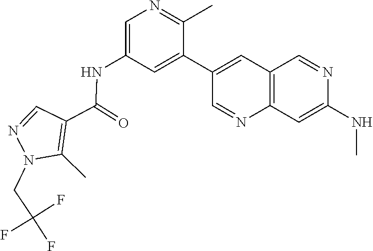 [SMILES: CNc1cc2ncc(-c3cc(NC(=O)c4cnn(CC(F)(F)F)c4C)cnc3C)cc2cn1] |
| 241 | 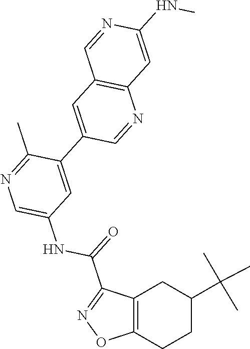 [SMILES: CNc1cc2ncc(-c3cc(NC(=O)c4noc5c4CC(C(C)(C)C)CC5)cnc3C)cc2cn1] |
| 242 | 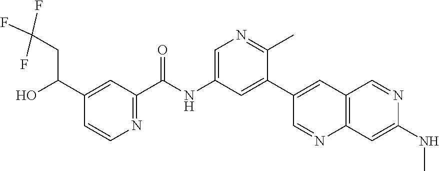 [SMILES: CNc1cc2ncc(-c3cc(NC(=O)c4cc(C(O)CC(F)(F)F)ccn4)cnc3C)cc2cn1] |
| 243 | 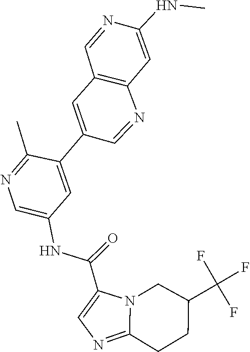 [SMILES: CNc1cc2ncc(-c3cc(NC(=O)c4cnc5n4CC(C(F)(F)F)CC5)cnc3C)cc2cn1] |
| 244 | 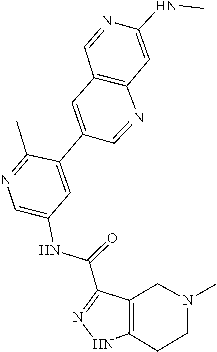 [SMILES: CNc1cc2ncc(-c3cc(NC(=O)c4n[nH]c5c4CN(C)CC5)cnc3C)cc2cn1] |
| 245 | 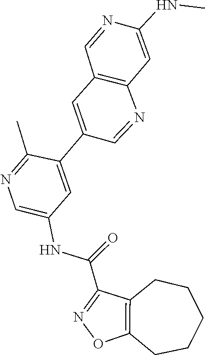 [SMILES: CNc1cc2ncc(-c3cc(NC(=O)c4noc5c4CCCCC5)cnc3C)cc2cn1] |
| 246 | 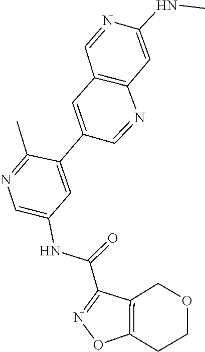 [SMILES: CNc1cc2ncc(-c3cc(NC(=O)c4noc5c4COCC5)cnc3C)cc2cn1] |
| 247 | 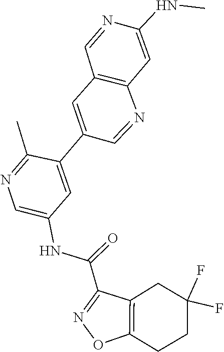 [SMILES: CNc1cc2ncc(-c3cc(NC(=O)c4noc5c4CC(F)(F)CC5)cnc3C)cc2cn1] |
| 248 | 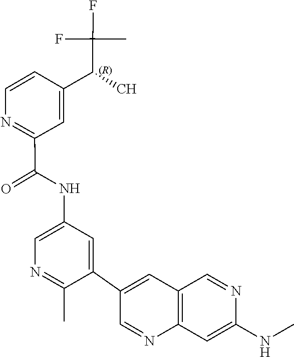 |
| 249 | 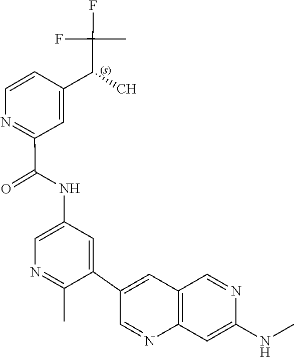 |
| 250 | 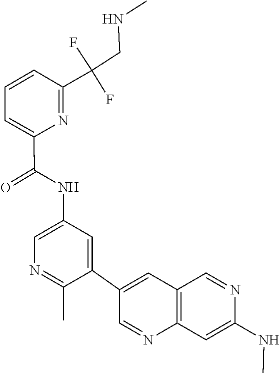 [SMILES: CNCC(F)(F)c1cccc(C(=O)Nc2cnc(C)c(-c3cnc4cc(NC)ncc4c3)c2)n1] |
| 251 | 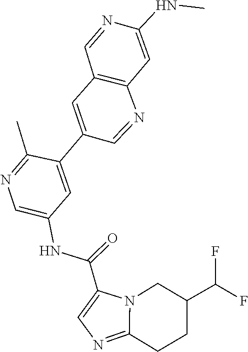 [SMILES: CNc1cc2ncc(-c3cc(NC(=O)c4cnc5n4CC(C(F)F)CC5)cnc3C)cc2cn1] |
| 252 | 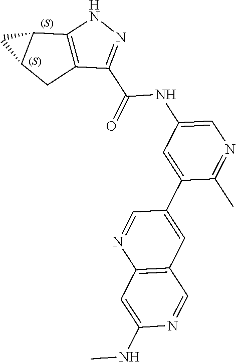 |
| 253 | 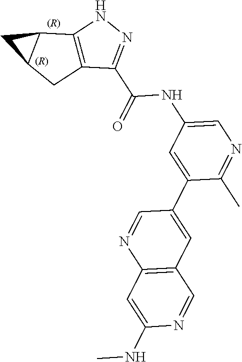 |
| 254 | 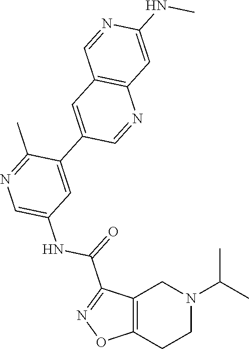 [SMILES: CNc1cc2ncc(-c3cc(NC(=O)c4noc5c4CN(C(C)C)CC5)cnc3C)cc2cn1] |
| 255 | 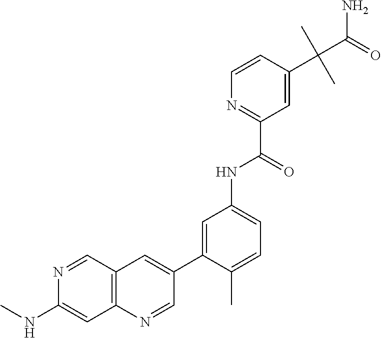 [SMILES: CNc1cc2ncc(-c3cc(NC(=O)c4cc(C(C)(C)C(N)=O)ccn4)ccc3C)cc2cn1] |
| 256 | 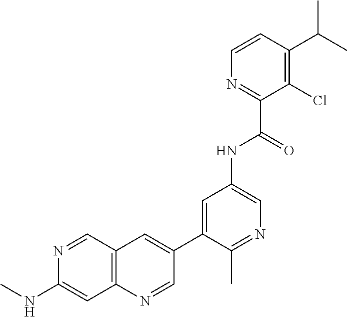 [SMILES: CNc1cc2ncc(-c3cc(NC(=O)c4nccc(C(C)C)c4Cl)cnc3C)cc2cn1] |
| 257 | 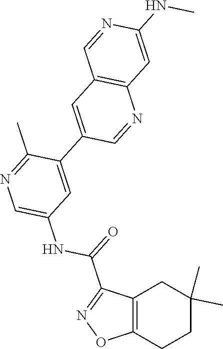 [SMILES: CNc1cc2ncc(-c3cc(NC(=O)c4noc5c4CC(C)(C)CC5)cnc3C)cc2cn1] |
| 258 | 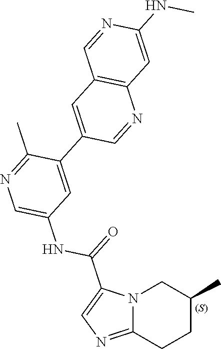 |
| 259 | 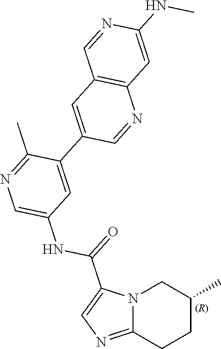 |
| 260 | 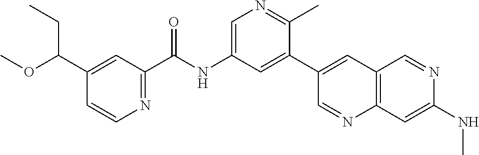 |
| 261 | 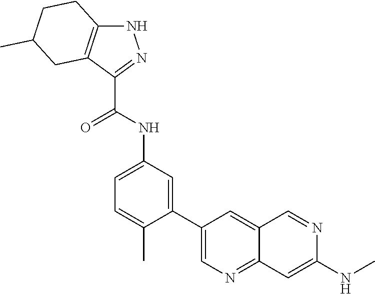 |
| 262 | 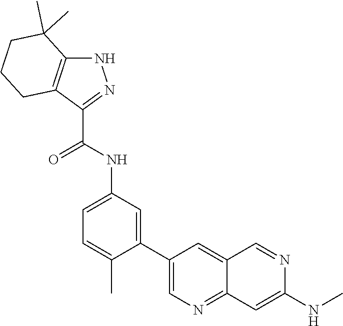 [SMILES: CNc1cc2ncc(-c3cc(NC(=O)c4n[nH]c5c4CCCC5(C)C)ccc3C)cc2cn1] |
| 263 | 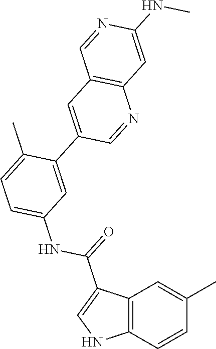 [SMILES: CNc1cc2ncc(-c3cc(NC(=O)c4c[nH]c5ccc(C)cc45)ccc3C)cc2cn1] |
| 264 | 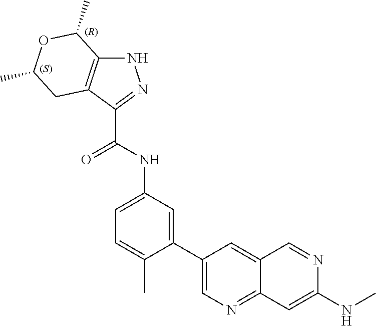 |
| 265 | 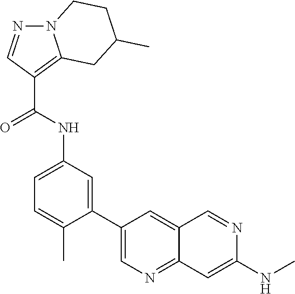 [SMILES: CNc1cc2ncc(-c3cc(NC(=O)c4cnn5c4CC(C)CC5)ccc3C)cc2cn1] |
| 266 | 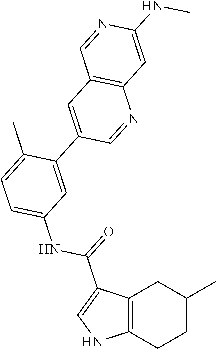 [SMILES: CNc1cc2ncc(-c3cc(NC(=O)c4c[nH]c5c4CC(C)CC5)ccc3C)cc2cn1] |
| 267 | 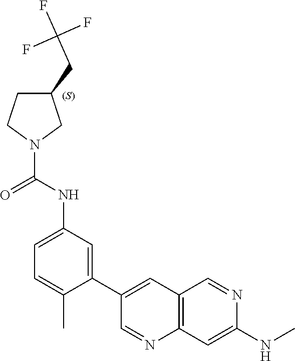 |
| 268 | 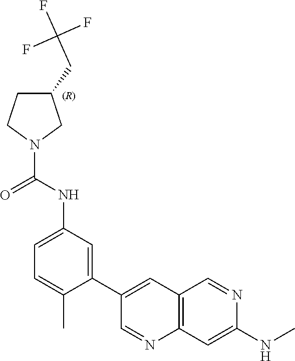 |
| 269 | 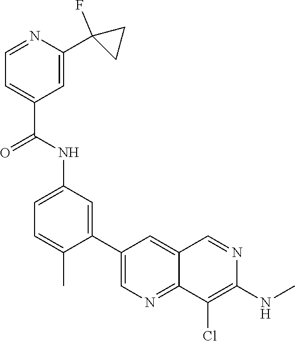 [SMILES: CNc1ncc2cc(-c3cc(NC(=O)c4ccnc(C5(F)CC5)c4)ccc3C)cnc2c1Cl] |
| 270 | 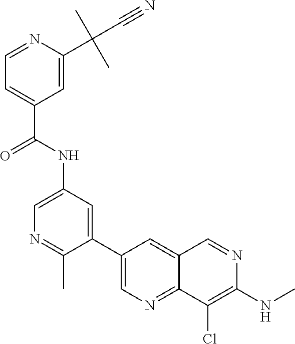 [SMILES: CNc1ncc2cc(-c3cc(NC(=O)c4ccnc(C(C)(C)C#N)c4)cnc3C)cnc2c1Cl] |
| 271 | 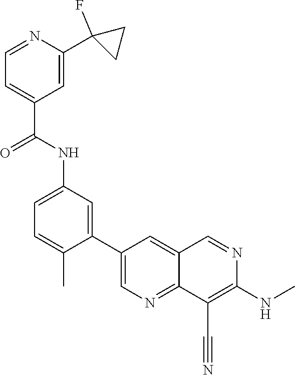 [SMILES: CNc1ncc2cc(-c3cc(NC(=O)c4ccnc(C5(F)CC5)c4)ccc3C)cnc2c1C#N] |
| 272 | 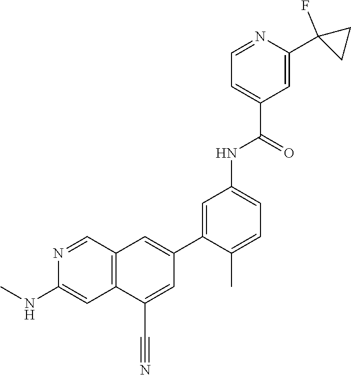 [SMILES: CNc1cc2c(C#N)cc(-c3cc(NC(=O)c4ccnc(C5(F)CC5)c4)ccc3C)cc2cn1] |
| 273 | 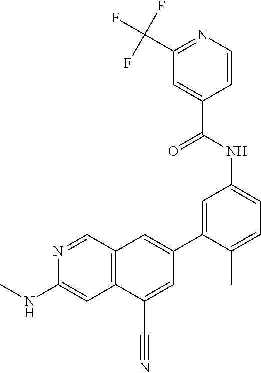 [SMILES: CNc1cc2c(C#N)cc(-c3cc(NC(=O)c4ccnc(C(F)(F)F)c4)ccc3C)cc2cn1] |
| 274 | 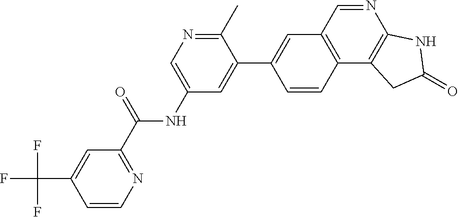 [SMILES: Cc1ncc(NC(=O)c2cc(C(F)(F)F)ccn2)cc1-c1ccc2c3c(ncc2c1)NC(=O)C3] |
| 275 | 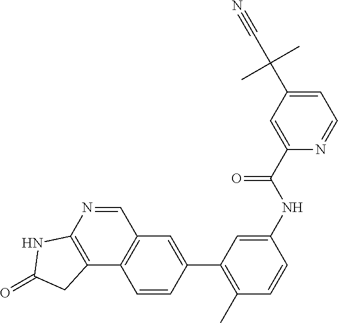 [SMILES: Cc1ccc(NC(=O)c2cc(C(C)(C)C#N)ccn2)cc1-c1ccc2c3c(ncc2c1)NC(=O)C3] |
| 276 | 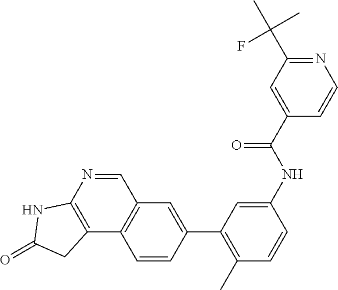 [SMILES: Cc1ccc(NC(=O)c2ccnc(C(C)(C)F)c2)cc1-c1ccc2c3c(ncc2c1)NC(=O)C3] |
| 277 | 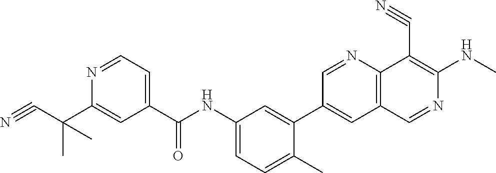 [SMILES: CNc1ncc2cc(-c3cc(NC(=O)c4ccnc(C(C)(C)C#N)c4)ccc3C)cnc2c1C#N] |
| 278 | 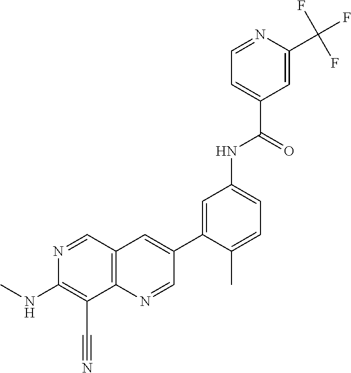 [SMILES: CNc1ncc2cc(-c3cc(NC(=O)c4ccnc(C(F)(F)F)c4)ccc3C)cnc2c1C#N] |
| 279 | 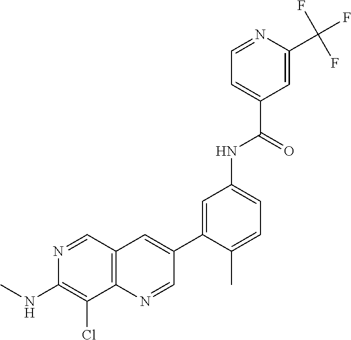 [SMILES: CNc1ncc2cc(-c3cc(NC(=O)c4ccnc(C(F)(F)F)c4)ccc3C)cnc2c1Cl] |
| 280 | 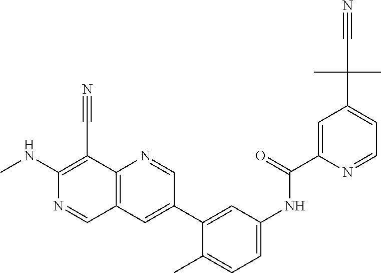 [SMILES: CNc1ncc2cc(-c3cc(NC(=O)c4cc(C(C)(C)C#N)ccn4)ccc3C)cnc2c1C#N] |
| 281 | 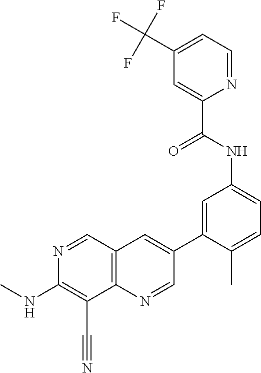 [SMILES: CNc1ncc2cc(-c3cc(NC(=O)c4cc(C(F)(F)F)ccn4)ccc3C)cnc2c1C#N] |
| 282 | 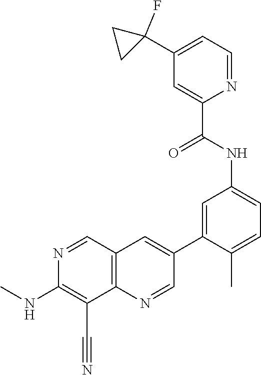 [SMILES: CNc1ncc2cc(-c3cc(NC(=O)c4cc(C5(F)CC5)ccn4)ccc3C)cnc2c1C#N] |
| 283 | 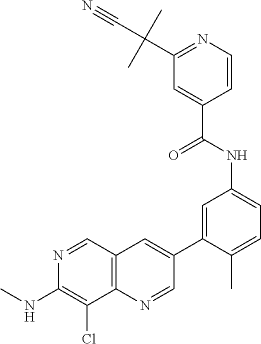 [SMILES: CNc1ncc2cc(-c3cc(NC(=O)c4ccnc(C(C)(C)C#N)c4)ccc3C)cnc2c1Cl] |
| 284 | 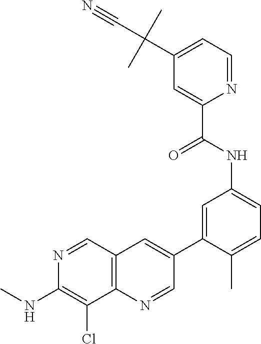 [SMILES: CNc1ncc2cc(-c3cc(NC(=O)c4cc(C(C)(C)C#N)ccn4)ccc3C)cnc2c1Cl] |
| 285 | 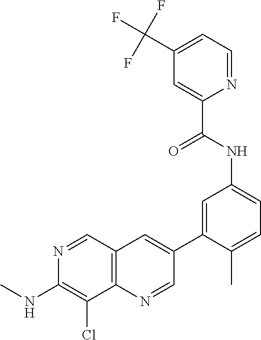 [SMILES: CNc1ncc2cc(-c3cc(NC(=O)c4cc(C(F)(F)F)ccn4)ccc3C)cnc2c1Cl] |
| 286 | 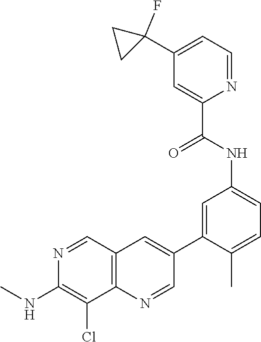 [SMILES: CNc1ncc2cc(-c3cc(NC(=O)c4cc(C5(F)CC5)ccn4)ccc3C)cnc2c1Cl] |
| 287 | 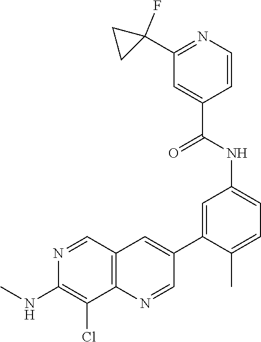 [SMILES: CNc1ncc2cc(-c3cc(NC(=O)c4ccnc(C5(F)CC5)c4)ccc3C)cnc2c1Cl] |
| 288 | 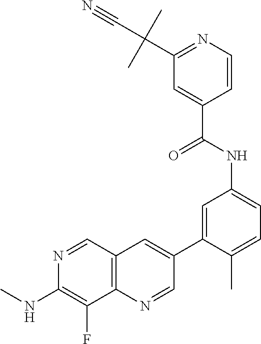 [SMILES: CNc1ncc2cc(-c3cc(NC(=O)c4ccnc(C(C)(C)C#N)c4)ccc3C)cnc2c1F] |
| 289 | 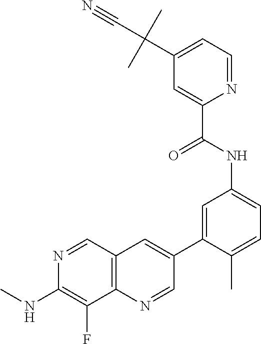 [SMILES: CNc1ncc2cc(-c3cc(NC(=O)c4cc(C(C)(C)C#N)ccn4)ccc3C)cnc2c1F] |
| 290 | 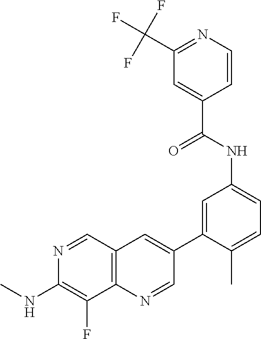 [SMILES: CNc1ncc2cc(-c3cc(NC(=O)c4ccnc(C(F)(F)F)c4)ccc3C)cnc2c1F] |
| 291 | 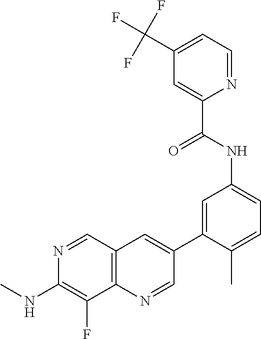 [SMILES: CNc1ncc2cc(-c3cc(NC(=O)c4cc(C(F)(F)F)ccn4)ccc3C)cnc2c1F] |
| 292 | 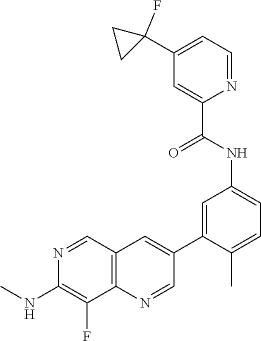 [SMILES: CNc1ncc2cc(-c3cc(NC(=O)c4cc(C5(F)CC5)ccn4)ccc3C)cnc2c1F] |
| 293 | 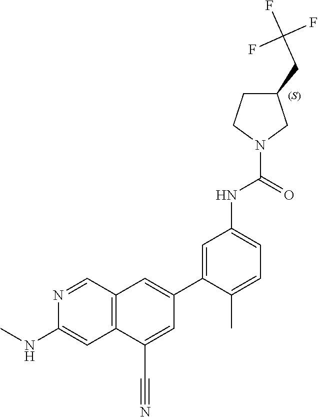 |
| 294 | 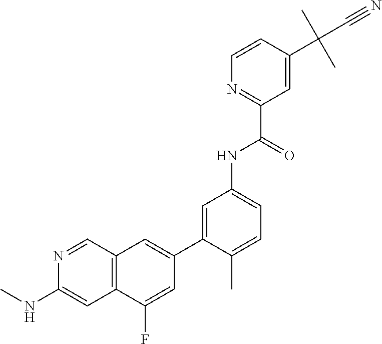 [SMILES: CNc1cc2c(F)cc(-c3cc(NC(=O)c4cc(C(C)(C)C#N)ccn4)ccc3C)cc2cn1] |
| 295 | 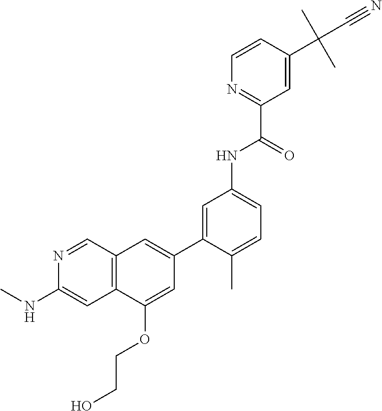 [SMILES: CNc1cc2c(OCCO)cc(-c3cc(NC(=O)c4cc(C(C)(C)C#N)ccn4)ccc3C)cc2cn1] |
| 296 | 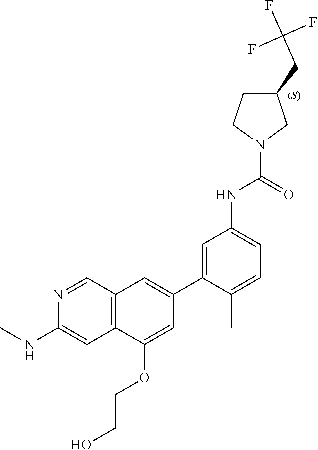 |
| 297 | 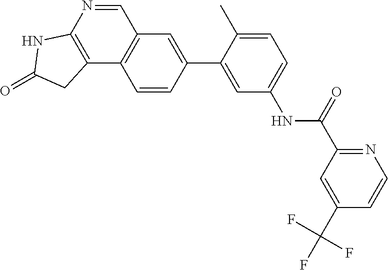 [SMILES: Cc1ccc(NC(=O)c2cc(C(F)(F)F)ccn2)cc1-c1ccc2c3c(ncc2c1)NC(=O)C3] |
| 298 | 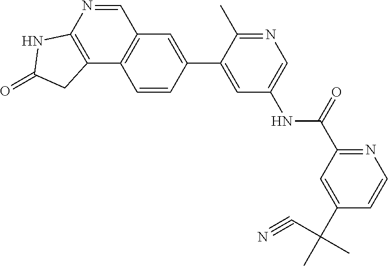 [SMILES: Cc1ncc(NC(=O)c2cc(C(C)(C)C#N)ccn2)cc1-c1ccc2c3c(ncc2c1)NC(=O)C3] |
| 299 | 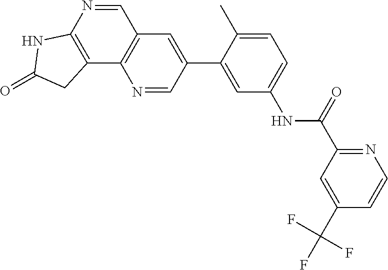 [SMILES: Cc1ccc(NC(=O)c2cc(C(F)(F)F)ccn2)cc1-c1cnc2c3c(ncc2c1)NC(=O)C3] |
| 300 | 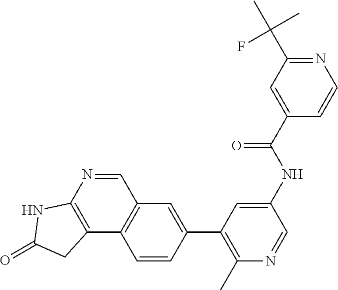 [SMILES: Cc1ncc(NC(=O)c2ccnc(C(C)(C)F)c2)cc1-c1ccc2c3c(ncc2c1)NC(=O)C3] |
| 301 | 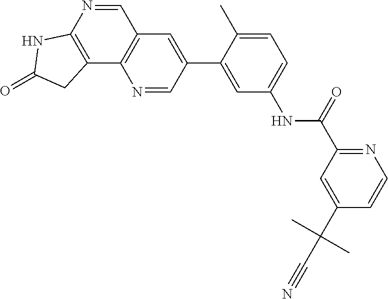 [SMILES: Cc1ccc(NC(=O)c2cc(C(C)(C)C#N)ccn2)cc1-c1cnc2c3c(ncc2c1)NC(=O)C3] |
| 302 | 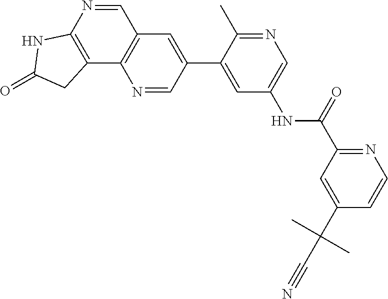 [SMILES: Cc1ncc(NC(=O)c2cc(C(C)(C)C#N)ccn2)cc1-c1cnc2c3c(ncc2c1)NC(=O)C3] |
| 303 | 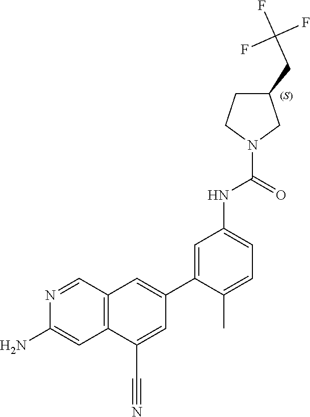 |
| 304 | 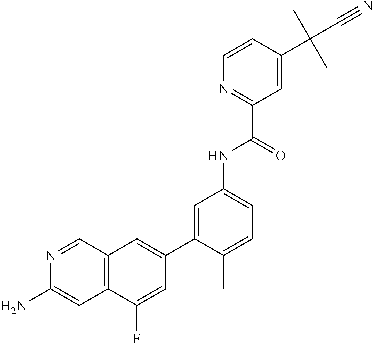 [SMILES: Cc1ccc(NC(=O)c2cc(C(C)(C)C#N)ccn2)cc1-c1cc(F)c2cc(N)ncc2c1] |
| 305 | 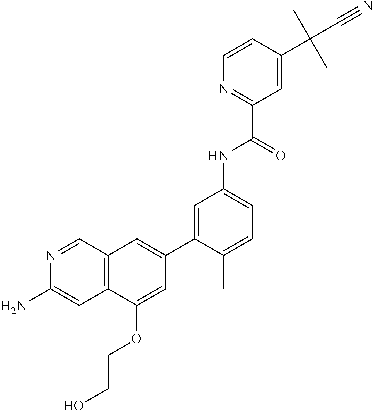 [SMILES: Cc1ccc(NC(=O)c2cc(C(C)(C)C#N)ccn2)cc1-c1cc(OCCO)c2cc(N)ncc2c1] |
| 306 | 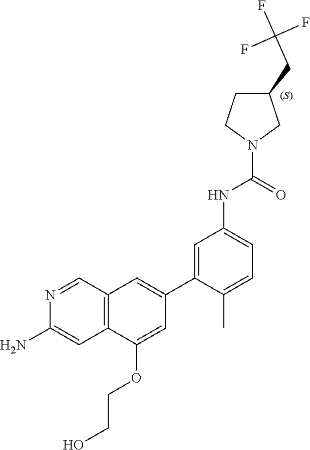 |
| 307 | 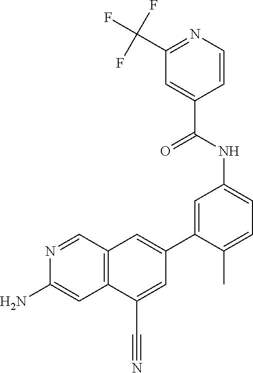 [SMILES: Cc1ccc(NC(=O)c2ccnc(C(F)(F)F)c2)cc1-c1cc(C#N)c2cc(N)ncc2c1] |
| 308 | 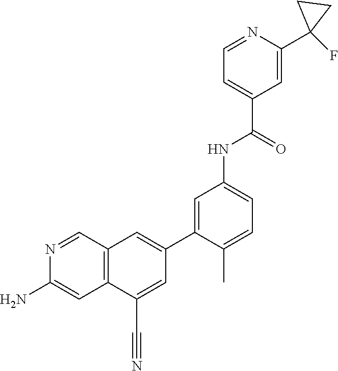 [SMILES: Cc1ccc(NC(=O)c2ccnc(C3(F)CC3)c2)cc1-c1cc(C#N)c2cc(N)ncc2c1] |
| 309 | 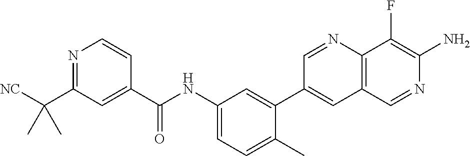 [SMILES: Cc1ccc(NC(=O)c2ccnc(C(C)(C)C#N)c2)cc1-c1cnc2c(F)c(N)ncc2c1] |
| 310 | 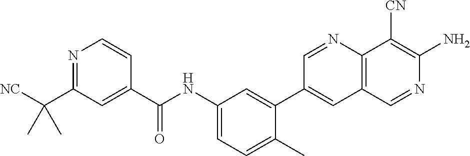 [SMILES: Cc1ccc(NC(=O)c2ccnc(C(C)(C)C#N)c2)cc1-c1cnc2c(C#N)c(N)ncc2c1] |
| 311 | 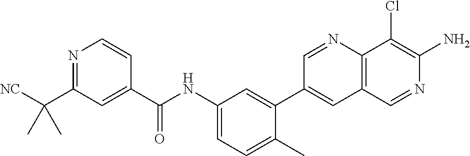 [SMILES: Cc1ccc(NC(=O)c2ccnc(C(C)(C)C#N)c2)cc1-c1cnc2c(Cl)c(N)ncc2c1] |
| 312 | 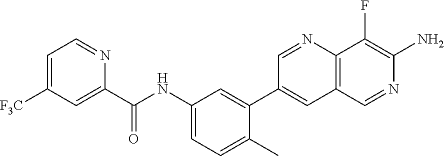 [SMILES: Cc1ccc(NC(=O)c2cc(C(F)(F)F)ccn2)cc1-c1cnc2c(F)c(N)ncc2c1] |
| 313 | 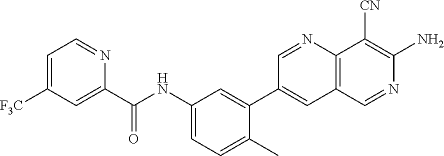 [SMILES: Cc1ccc(NC(=O)c2cc(C(F)(F)F)ccn2)cc1-c1cnc2c(C#N)c(N)ncc2c1] |
| 314 | 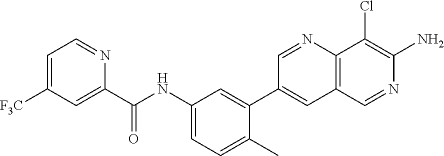 [SMILES: Cc1ccc(NC(=O)c2cc(C(F)(F)F)ccn2)cc1-c1cnc2c(Cl)c(N)ncc2c1] |
| 315 | 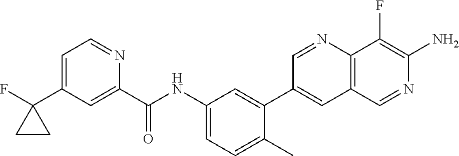 [SMILES: Cc1ccc(NC(=O)c2cc(C3(F)CC3)ccn2)cc1-c1cnc2c(F)c(N)ncc2c1] |
| 316 | 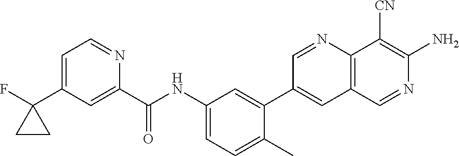 [SMILES: Cc1ccc(NC(=O)c2cc(C3(F)CC3)ccn2)cc1-c1cnc2c(C#N)c(N)ncc2c1] |
| 317 | 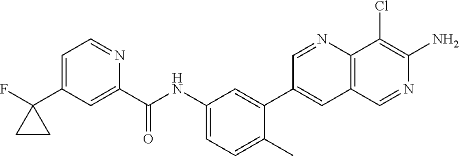 [SMILES: Cc1ccc(NC(=O)c2cc(C3(F)CC3)ccn2)cc1-c1cnc2c(Cl)c(N)ncc2c1] |
| 318 | 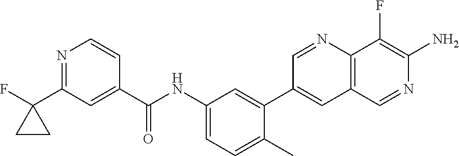 [SMILES: Cc1ccc(NC(=O)c2ccnc(C3(F)CC3)c2)cc1-c1cnc2c(F)c(N)ncc2c1] |
| 319 | 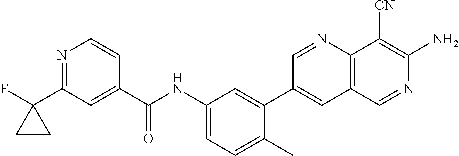 [SMILES: Cc1ccc(NC(=O)c2ccnc(C3(F)CC3)c2)cc1-c1cnc2c(C#N)c(N)ncc2c1] |
| 320 | 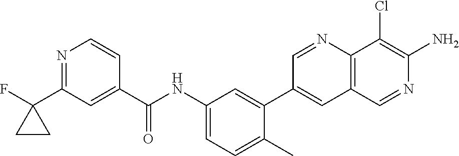 [SMILES: Cc1ccc(NC(=O)c2ccnc(C3(F)CC3)c2)cc1-c1cnc2c(Cl)c(N)ncc2c1] |
| 321 | 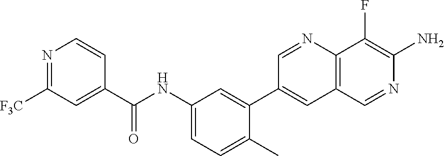 [SMILES: Cc1ccc(NC(=O)c2ccnc(C(F)(F)F)c2)cc1-c1cnc2c(F)c(N)ncc2c1] |
| 322 | 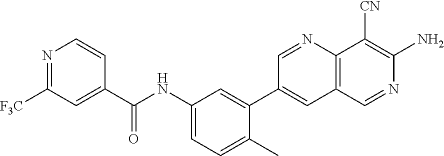 [SMILES: Cc1ccc(NC(=O)c2ccnc(C(F)(F)F)c2)cc1-c1cnc2c(C#N)c(N)ncc2c1] |
| 323 | 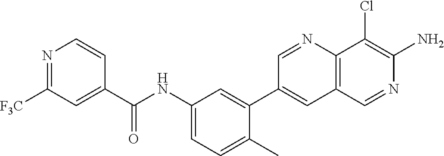 [SMILES: Cc1ccc(NC(=O)c2ccnc(C(F)(F)F)c2)cc1-c1cnc2c(Cl)c(N)ncc2c1] |
| 324 | 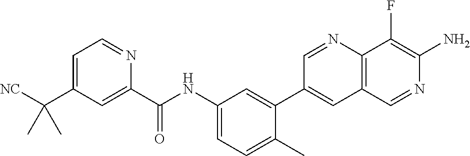 [SMILES: Cc1ccc(NC(=O)c2cc(C(C)(C)C#N)ccn2)cc1-c1cnc2c(F)c(N)ncc2c1] |
| 325 | 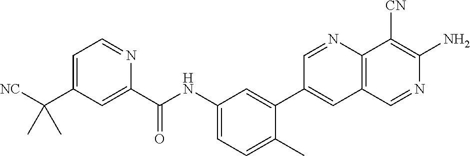 [SMILES: Cc1ccc(NC(=O)c2cc(C(C)(C)C#N)ccn2)cc1-c1cnc2c(C#N)c(N)ncc2c1] |
| 326 | 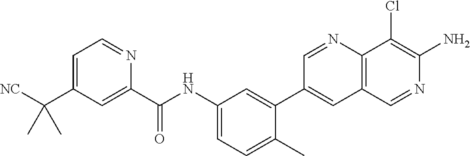 [SMILES: Cc1ccc(NC(=O)c2cc(C(C)(C)C#N)ccn2)cc1-c1cnc2c(Cl)c(N)ncc2c1] |

In some embodiments, the compound exhibits an inhibition activity against a Class II mutation or a Class III mutation.

In some embodiments, the compound exhibits an inhibition activity against a Class II mutation or a Class III mutation that is higher than a comparable agent (e.g., encorafenib), as measured in IC₅₀ value.

In some embodiments, the compound exhibits an inhibition activity against a Class II mutation or a Class III mutation that is more than three-fold, more than four-fold, more than five-fold, or more than ten-fold higher than a comparable agent (e.g., encorafenib), as measured in IC₅₀ value.

In some aspects, the present disclosure provides a compound being an isotopic derivative (e.g., isotopically labeled compound) of any one of the compounds disclosed herein.

In some embodiments, the compound is an isotopic derivative of any one of the compounds described in Table I or Table II, or a pharmaceutically acceptable salt thereof.

In some embodiments, the compound is an isotopic derivative of any one of the compounds described in Table I or Table II.

In some embodiments, the compound is an isotopic derivative of any one of the compounds described in Table I, or a pharmaceutically acceptable salt thereof.

In some embodiments, the compound is an isotopic derivative of any one of the compounds described in Table I.

In some embodiments, the compound is an isotopic derivative of any one of the compounds described in Table II, or a pharmaceutically acceptable salt thereof.

In some embodiments, the compound is an isotopic derivative of any one of the compounds described in Table II.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table I or Table IT, or a pharmaceutically acceptable salt thereof.

In some embodiments, the compound is a deuterium labeled compound of any one of the compounds described in Table I or Table IT.

In some embodiments, the compound is a deuterium labeled compound of any one of the compounds described in Table I, or a pharmaceutically acceptable salt thereof.

In some embodiments, the compound is a deuterium labeled compound of any one of the compounds described in Table I.

In some embodiments, the compound is a deuterium labeled compound of any one of the compounds described in Table II, or a pharmaceutically acceptable salt thereof.

In some embodiments, the compound is a deuterium labeled compound of any one of the compounds described in Table II.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the present disclosure or a pharmaceutically acceptable salt or solvate thereof that contains the aforementioned deuterium atom(s) is within the scope of the disclosure.

Further, substitution with deuterium (i.e., ²H) may afford certain therapeutic advantages resulting from greater metabolic stability, e.g., increased in vivo half-life or reduced dosage requirements.

For the avoidance of doubt it is to be understood that, where in this specification a group is qualified by “described herein”, the said group encompasses the first occurring and broadest definition as well as each and all of the particular definitions for that group.

A suitable pharmaceutically acceptable salt of a compound of the disclosure is, for example, an acid-addition salt of a compound of the disclosure which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of the present disclosure and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centers (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of the present disclosure may exist in solvated as well as unsolvated forms such as, for example, hydrated forms. A suitable pharmaceutically acceptable solvate is, for example, a hydrate such as hemi-hydrate, a monohydrate, a di-hydrate or a tri-hydrate. It is to be understood that the disclosure encompasses all such solvated forms that possess inflammasome inhibitory activity.

It is also to be understood that certain compounds of the present disclosure may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of the present disclosure may exist in a number of different tautomeric forms and references to compounds of the present disclosure include all such forms. For the avoidance of doubt, where a compound can exist in one of several tautomeric forms, and only one is specifically described or shown, all others are nevertheless embraced by the Formulae disclosed. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

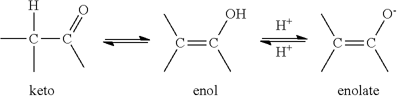

Compounds of the present disclosure containing an amine function may also form N-oxides. A reference herein to a compound disclosed herein that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of the present disclosure may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the sulfonylurea group in a compound of the any one of the Formulae disclosed herein.

Accordingly, the present disclosure includes those compounds of the present disclosure as defined hereinbefore when made available by organic synthesis and when made available within the human or animal body by way of cleavage of a prodrug thereof. Accordingly, the present disclosure includes those compounds of the present disclosure that are produced by organic synthetic means and also such compounds that are produced in the human or animal body by way of metabolism of a precursor compound, that is a compound of the present disclosure may be a synthetically-produced compound or a metabolically-produced compound.

A suitable pharmaceutically acceptable prodrug of a compound of the present disclosure is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of the present disclosure that possesses a hydroxy group is, for example, an in vivo cleavable ester or ether thereof. An in vivo cleavable ester or ether of a compound of the present disclosure containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of a compound of the present disclosure that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of the present disclosure that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl, and 4-(C₁-C₄ alkyl)piperazin-1-ylmethyl.

The in vivo effects of a compound of the present disclosure may be exerted in part by one or more metabolites that are formed within the human or animal body after administration of a compound of the present disclosure. As stated hereinbefore, the in vivo effects of a compound of the present disclosure may also be exerted by way of metabolism of a precursor compound (a prodrug).

### Methods of Synthesis

In some aspects, the present disclosure provides a method of preparing a compound disclosed herein.

In some aspects, the present disclosure provides a method of preparing a compound, comprising one or more steps as described herein.

In some aspects, the present disclosure provides a compound obtainable by, or obtained by, or directly obtained by a method for preparing a compound described herein.

In some aspects, the present disclosure provides an intermediate being suitable for use in a method for preparing a compound described herein.

The compounds of the present disclosure can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl, or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium on carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium on carbon.

Once a compound of the present disclosure has been synthesized by any one of the processes defined herein, the processes may then further comprise the additional steps of: (i) removing any protecting groups present; (ii) converting the compound of the present disclosure into another compound of the present disclosure; (iii) forming a pharmaceutically acceptable salt, hydrate or solvate thereof; and/or (iv) forming a prodrug thereof.

The resultant compounds of the present disclosure can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

The reaction temperature is suitably between about −100° C. and 300° C., depending on the reaction step and the conditions used.

Reaction times are generally in the range between a fraction of a minute and several days, depending on the reactivity of the respective compounds and the respective reaction conditions. Suitable reaction times are readily determinable by methods known in the art, for example reaction monitoring. Based on the reaction temperatures given above, suitable reaction times generally lie in the range between 10 minutes and 48 hours.

Moreover, by utilizing the procedures described herein, in conjunction with ordinary skills in the art, additional compounds of the present disclosure can be readily prepared. Those skilled in the art will readily understand that known variations of the conditions and processes of the following preparative procedures can be used to prepare these compounds.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful—in order to obtain the compounds of the present disclosure. Furthermore, some of the compounds of the present disclosure can readily be synthesized by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person. Likewise, the skilled person will apply-whenever necessary or useful-synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

General routes for the preparation of a compound of the application are described in Schemes I-X.

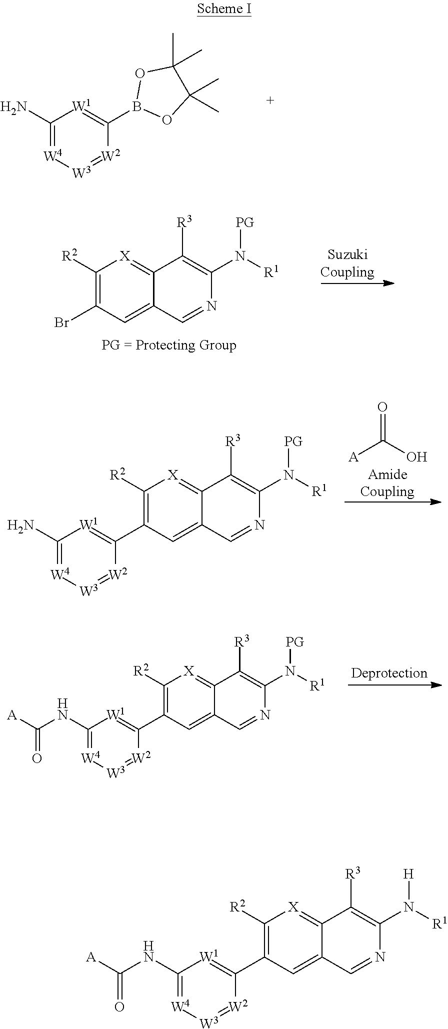

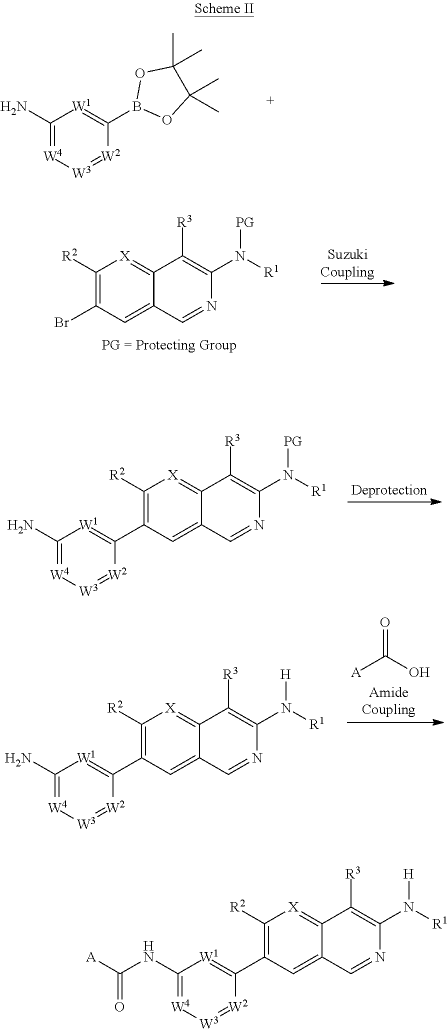

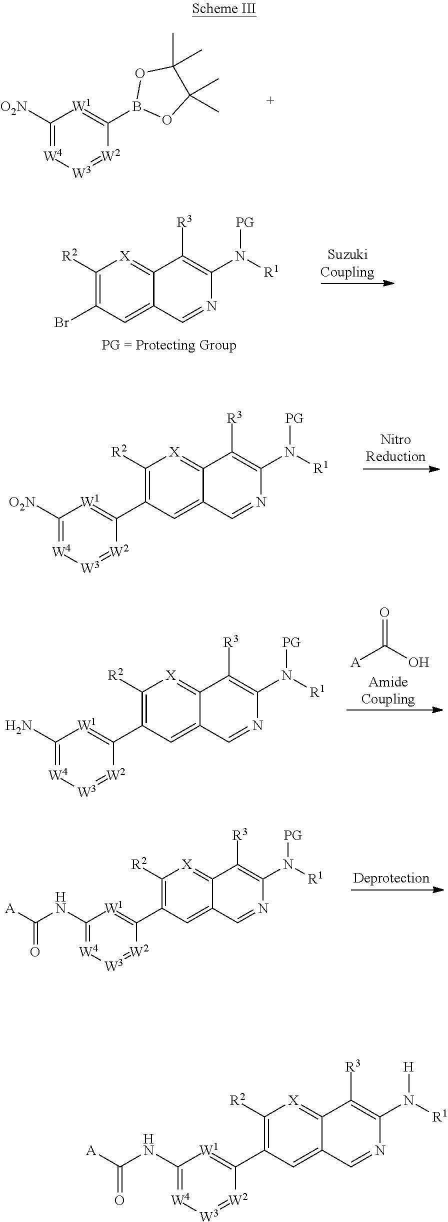

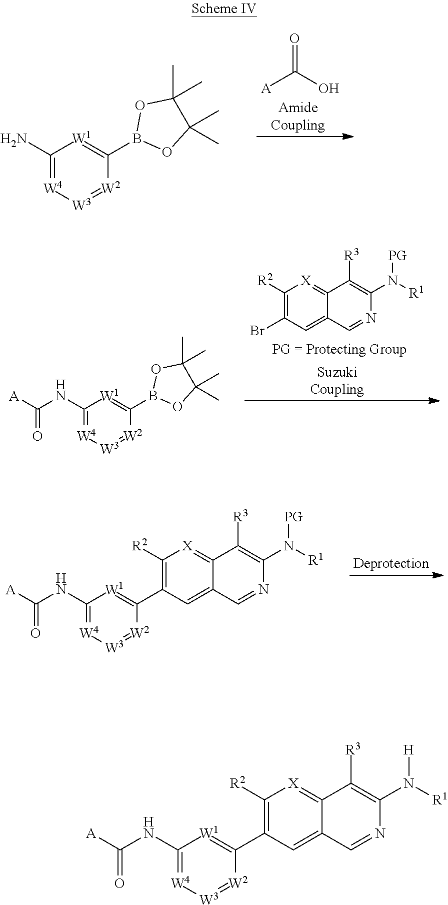

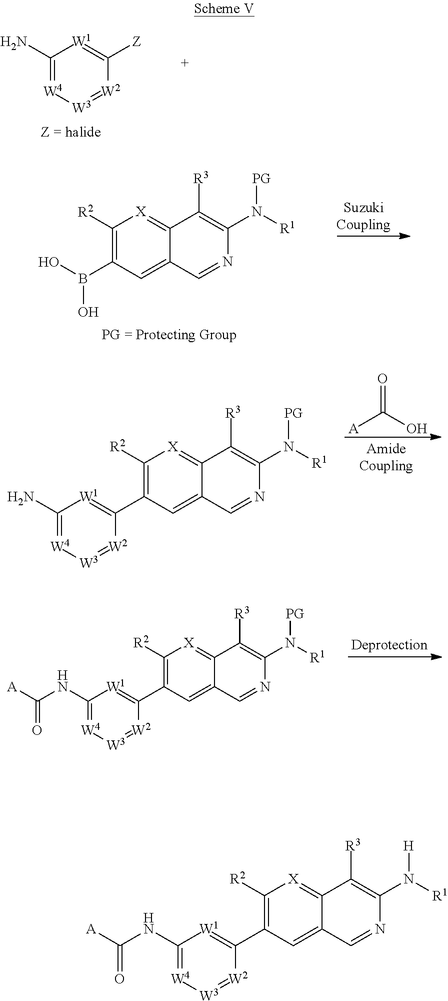

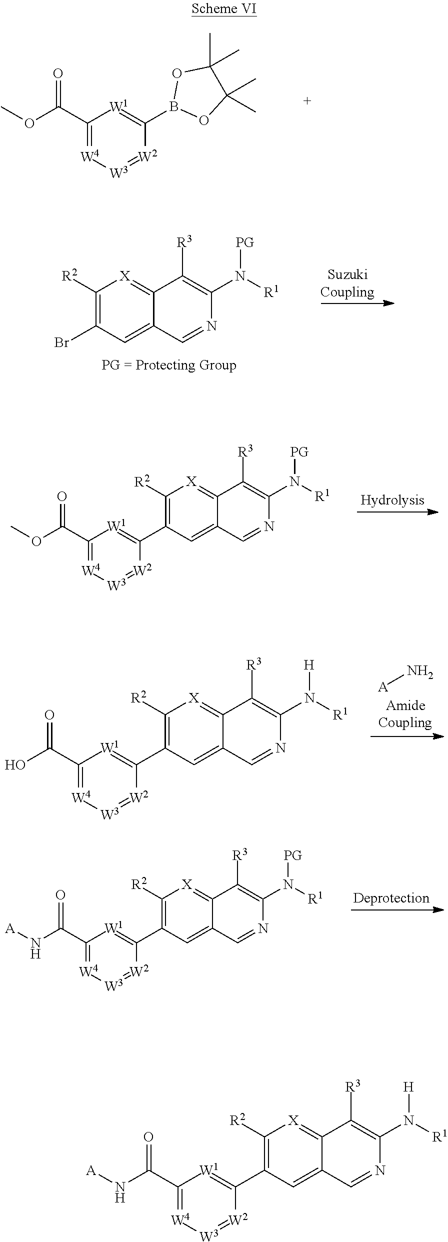

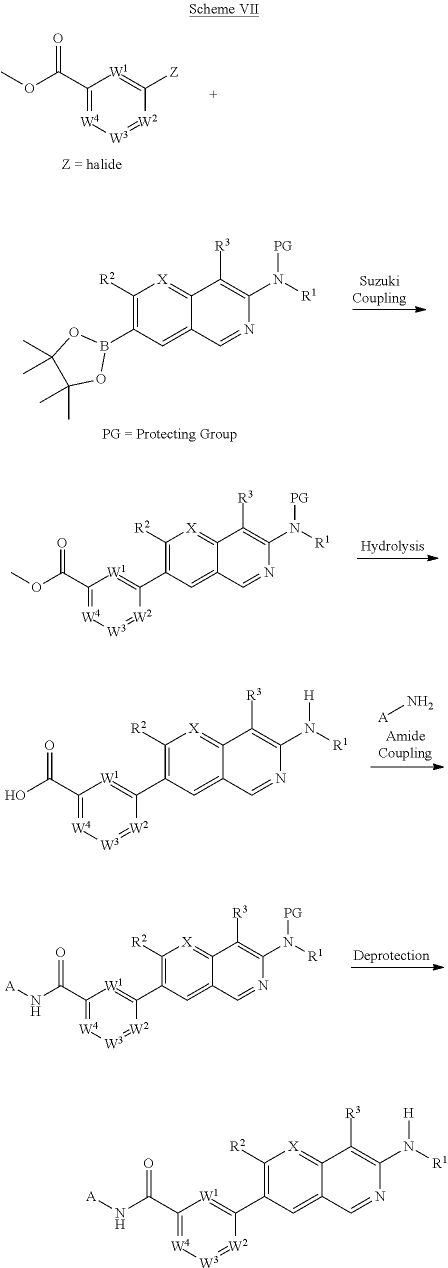

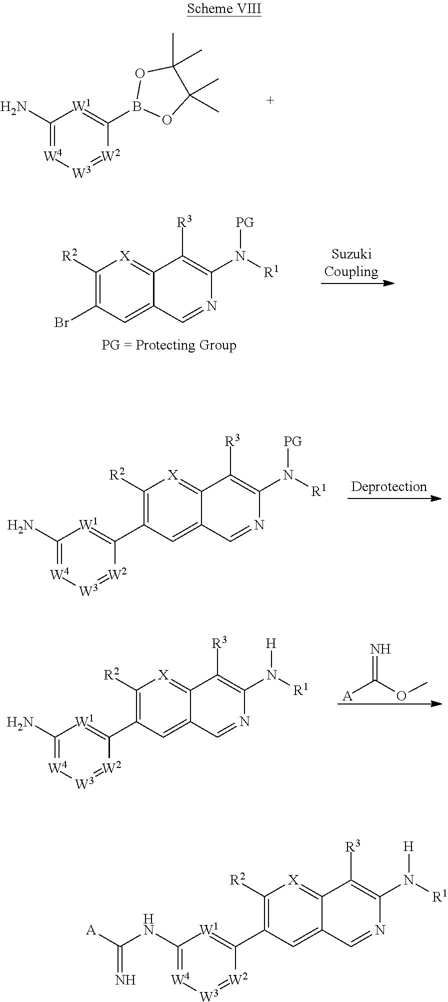

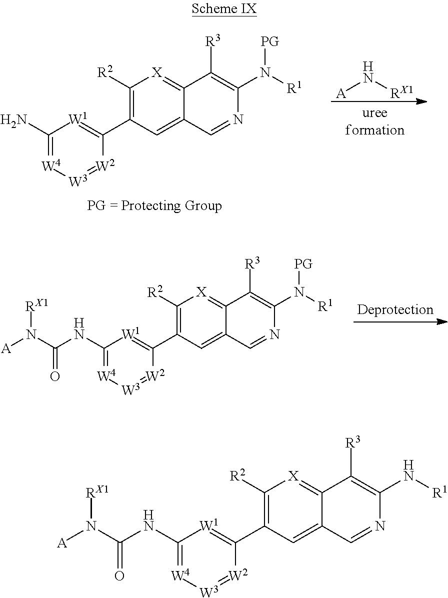

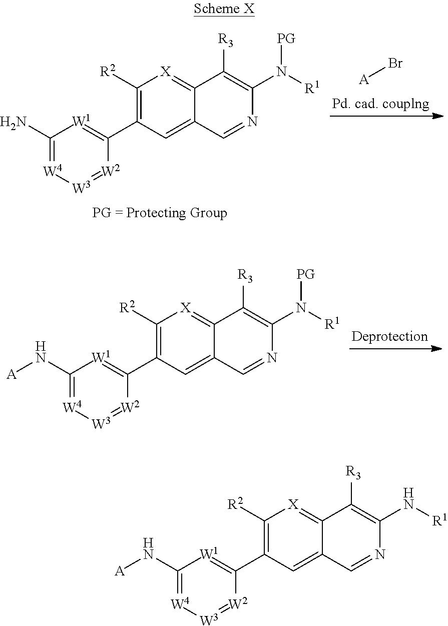

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

In some embodiments the biological assay may involve retroviral production.

In some embodiments, a fusion mutant (e.g., BRAF-KIAA1549) may be subcloned into a retroviral expression vector (e.g., pMXs-IRES-Blasticidin), wherein the retrovirus may be produced by transfection of cells (e.g., HEK 293T) with retroviral plasmids (e.g., retroviral BRAF mutant expression vector).

In some embodiments, the cells (e.g., HEK 293T) may be plated and incubated. In some embodiments, the retroviral plasmids (e.g., BRAF-KIAA fusion mutant) may added to a transfection reagent and then added to cells (e.g., HEK 293T), wherein the cells may be harvested.

In some embodiments, the biological assay may involve the generation of a fusion stable cell line (e.g., a BRAF-KIAA1549 fusion stable cell line).

In some embodiments, cells (e.g., BaF3) may be transduced with a viral supernatant (e.g., BRAF-KIAA1549 fusion viral supernatant) and the cells may be sampled for viability (e.g., by Luminescent Cell Viability Assay such as CellTiterGlo). In some embodiments, the fusion stable cell line may undergo cell banking and sequence confirmation (e.g., sanger sequencing).

In some embodiments, the biological assay is for cell proliferation.

In some embodiments, cells (e.g., BaF3 BRAF-KIAA1549 fusion cells) are suspended and dispensed in plates. In some embodiments, to determine the effect of compounds of the present disclosure on cell proliferation, the cells (e.g., BaF3 BRAF-KIAA1549 fusion cells) may be incubated in the presence of vehicle control (e.g., DMSO) or a compound of the present disclosure at varying concentrations and the inhibition of cell growth may be determined by luminescent quantification (e.g., of intracellular ATP content using CellTiterGlo), according to the manufacturers protocol. In some embodiments, to determine the IC₅₀ values, the vehicle-treated cells were normalized as viable cells and analyzed using a software (e.g., the CDD Vault (Collaborative Drug Discovery, Burlingame, CA) using an algorithm (e.g., the Levenberg-Marquardt algorithm; Levenberg, K., 1994; Marquardt, D., 1963).

### Pharmaceutical Compositions

In some aspects, the present disclosure provides a pharmaceutical composition comprising a compound of the present disclosure as an active ingredient.

In some embodiments, the present disclosure provides a pharmaceutical composition comprising a compound described herein and one or more pharmaceutically acceptable carriers or excipients. In some embodiments, the present disclosure provides a pharmaceutical composition comprising at least one compound selected from Table I and Table II. In some embodiments, the present disclosure provides a pharmaceutical composition comprising at least one compound selected from Table I. In some embodiments, the present disclosure provides a pharmaceutical composition comprising at least one compound selected from Table II.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the present disclosure, or a pharmaceutically acceptable salt, hydrate or solvate thereof, in association with a pharmaceutically acceptable diluent or carrier.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

An effective amount of a compound of the present disclosure for use in therapy is an amount sufficient to treat or prevent an inflammasome related condition referred to herein, slow its progression and/or reduce the symptoms associated with the condition.

The size of the dose for therapeutic or prophylactic purposes of a compound of the present disclosure will naturally vary according to the nature and severity of the conditions, the age and sex of the animal, subject, or patient and the route of administration, according to well-known principles of medicine.

### Methods of Use

In some aspects, the present disclosure provides methods of treating or preventing cancer in a subject, the method comprising administering to the subject a therapeutically effective amount of at least one compound of the present disclosure, or a pharmaceutically acceptable salt thereof.

In some aspects, the present disclosure provides a method of treating or preventing cancer in a subject, the method comprising administering to the subject a therapeutically effective amount of a compound of the present disclosure, or a pharmaceutically acceptable salt thereof.

In some aspects, the present disclosure provides a method of treating or preventing cancer in a subject, the method comprising administering to the subject a compound of the present disclosure, or a pharmaceutically acceptable salt thereof.

In some aspects, the present disclosure provides methods of treating cancer in a subject, the method comprising administering to the subject a therapeutically effective amount of at least one compound of the present disclosure, or a pharmaceutically acceptable salt thereof.

In some aspects, the present disclosure provides methods of treating cancer in a subject, the method comprising administering to the subject a therapeutically effective amount of a compound of the present disclosure, or a pharmaceutically acceptable salt thereof.

In some aspects, the present disclosure provides methods of treating cancer in a subject, the method comprising administering to the subject a compound of the present disclosure, or a pharmaceutically acceptable salt thereof.

In some aspects, the present disclosure provides at least one compound of the present disclosure, or a pharmaceutically acceptable salt thereof, for treating or preventing cancer in a subject.

In some aspects, the present disclosure provides a compound of the present disclosure, or a pharmaceutically acceptable salt thereof, for treating or preventing cancer in a subject.

In some aspects, the present disclosure provides at least one compound of the present disclosure, or a pharmaceutically acceptable salt thereof, for treating cancer in a subject.

In some aspects, the present disclosure provides a compound of the present disclosure, or a pharmaceutically acceptable salt thereof, for treating cancer in a subject.

In some aspects, the present disclosure provides a use of at least one compound of the present disclosure, or a pharmaceutically acceptable salt thereof, in the manufacture of a medicament for treating or preventing cancer in a subject.

In some aspects, the present disclosure provides a use of a compound of the present disclosure, or a pharmaceutically acceptable salt thereof, in the manufacture of a medicament for treating or preventing cancer in a subject.

In some aspects, the present disclosure provides a use of at least one compound of the present disclosure, or a pharmaceutically acceptable salt thereof, in the manufacture of a medicament for treating cancer in a subject.

In some aspects, the present disclosure provides a use of a compound of the present disclosure, or a pharmaceutically acceptable salt thereof, in the manufacture of a medicament for treating cancer in a subject.

In some aspects, the present disclosure provides a use of at least one compound of the present disclosure, or a pharmaceutically acceptable salt thereof, for treating or preventing cancer in a subject.

In some aspects, the present disclosure provides a use of a compound of the present disclosure, or a pharmaceutically acceptable salt thereof, for treating or preventing cancer in a subject.

In some aspects, the present disclosure provides a use of at least one compound of the present disclosure, or a pharmaceutically acceptable salt thereof, for treating cancer in a subject.

In some aspects, the present disclosure provides a use of a compound of the present disclosure, or a pharmaceutically acceptable salt thereof, for treating cancer in a subject.

In some aspects, the present disclosure provides methods of treating or preventing cancer in a subject, the method comprising administering to the subject a therapeutically effective amount of a pharmaceutical composition comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical composition comprising a compound of the present disclosure, or a pharmaceutically acceptable salt thereof.

In some aspects, the present disclosure provides methods of treating or preventing cancer in a subject, the method comprising administering to the subject a pharmaceutical composition comprising a compound of the present disclosure, or a pharmaceutically acceptable salt thereof.

In some aspects, the present disclosure provides methods of treating cancer in a subject, the method comprising administering to the subject a therapeutically effective amount of a pharmaceutical composition comprising at least one compound of the present disclosure, or a pharmaceutically acceptable salt thereof.

In some aspects, the present disclosure provides methods of treating cancer in a subject, the method comprising administering to the subject a therapeutically effective amount of a pharmaceutical composition comprising a compound of the present disclosure, or a pharmaceutically acceptable salt thereof.

In some aspects, the present disclosure provides methods of treating cancer in a subject, the method comprising administering to the subject a pharmaceutical composition comprising a compound of the present disclosure, or a pharmaceutically acceptable salt thereof.

In some aspects, the present disclosure provides a pharmaceutical composition comprising at least one compound of the present disclosure, or a pharmaceutically acceptable salt thereof, for treating or preventing cancer in a subject.

In some aspects, the present disclosure provides a pharmaceutical composition comprising a compound of the present disclosure, or a pharmaceutically acceptable salt thereof, for treating or preventing cancer in a subject.

In some aspects, the present disclosure provides a pharmaceutical composition comprising at least one compound of the present disclosure, or a pharmaceutically acceptable salt thereof, for treating cancer in a subject.

In some aspects, the present disclosure provides a pharmaceutical composition comprising a compound of the present disclosure, or a pharmaceutically acceptable salt thereof, for treating cancer in a subject.

In some aspects, the present disclosure provides a use of a pharmaceutical composition comprising at least one compound of the present disclosure, or a pharmaceutically acceptable salt thereof, in the manufacture of a medicament for treating or preventing cancer in a subject.

In some aspects, the present disclosure provides a use of a pharmaceutical composition comprising a compound of the present disclosure, or a pharmaceutically acceptable salt thereof, in the manufacture of a medicament for treating or preventing cancer in a subject.

In some aspects, the present disclosure provides a use of a pharmaceutical composition comprising at least one compound of the present disclosure, or a pharmaceutically acceptable salt thereof, in the manufacture of a medicament for treating cancer in a subject.

In some aspects, the present disclosure provides a use of a pharmaceutical composition comprising a compound of the present disclosure, or a pharmaceutically acceptable salt thereof, in the manufacture of a medicament for treating cancer in a subject.

In some aspects, the present disclosure provides a use of a pharmaceutical composition comprising at least one compound of the present disclosure, or a pharmaceutically acceptable salt thereof, for treating or preventing cancer in a subject.

In some aspects, the present disclosure provides a use of a pharmaceutical composition comprising a compound of the present disclosure, or a pharmaceutically acceptable salt thereof, for treating or preventing cancer in a subject.

In some aspects, the present disclosure provides a use of a pharmaceutical composition comprising at least one compound of the present disclosure, or a pharmaceutically acceptable salt thereof, for treating cancer in a subject.

In some aspects, the present disclosure provides a use of a pharmaceutical composition comprising a compound of the present disclosure, or a pharmaceutically acceptable salt thereof, for treating cancer in a subject.

In some aspects, the present disclosure provides methods of treating or preventing cancer in a subject, the method comprising administering to the subject a therapeutically effective amount of a pharmaceutical kit comprising at least one compound of the present disclosure, or a pharmaceutically acceptable salt thereof.

In some aspects, the present disclosure provides methods of treating or preventing cancer in a subject, the method comprising administering to the subject a therapeutically effective amount of a pharmaceutical kit comprising a compound of the present disclosure, or a pharmaceutically acceptable salt thereof.

In some aspects, the present disclosure provides methods of treating or preventing cancer in a subject, the method comprising administering to the subject a pharmaceutical kit comprising a compound of the present disclosure, or a pharmaceutically acceptable salt thereof.

In some aspects, the present disclosure provides methods of treating cancer in a subject, the method comprising administering to the subject a therapeutically effective amount of a pharmaceutical kit comprising at least one compound of the present disclosure, or a pharmaceutically acceptable salt thereof.

In some aspects, the present disclosure provides methods of treating cancer in a subject, the method comprising administering to the subject a therapeutically effective amount of a pharmaceutical kit comprising a compound of the present disclosure, or a pharmaceutically acceptable salt thereof.

In some aspects, the present disclosure provides methods of treating cancer in a subject, the method comprising administering to the subject a pharmaceutical kit comprising a compound of the present disclosure, or a pharmaceutically acceptable salt thereof.

In some aspects, the present disclosure provides a pharmaceutical kit comprising at least one compound of the present disclosure, or a pharmaceutically acceptable salt thereof, for treating or preventing cancer in a subject.

In some aspects, the present disclosure provides a pharmaceutical kit comprising a compound of the present disclosure, or a pharmaceutically acceptable salt thereof, for treating or preventing cancer in a subject.

In some aspects, the present disclosure provides a pharmaceutical kit comprising at least one compound of the present disclosure, or a pharmaceutically acceptable salt thereof, for treating cancer in a subject.

In some aspects, the present disclosure provides a pharmaceutical kit comprising a compound of the present disclosure, or a pharmaceutically acceptable salt thereof, for treating cancer in a subject.

In some aspects, the present disclosure provides a use of a pharmaceutical kit comprising at least one compound of the present disclosure, or a pharmaceutically acceptable salt thereof, in the manufacture of a medicament for treating or preventing cancer in a subject.

In some aspects, the present disclosure provides a use of a pharmaceutical kit comprising a compound of the present disclosure, or a pharmaceutically acceptable salt thereof, in the manufacture of a medicament for treating or preventing cancer in a subject.

In some aspects, the present disclosure provides a use of a pharmaceutical kit comprising at least one compound of the present disclosure, or a pharmaceutically acceptable salt thereof, in the manufacture of a medicament for treating cancer in a subject.

In some aspects, the present disclosure provides a use of a pharmaceutical kit comprising a compound of the present disclosure, or a pharmaceutically acceptable salt thereof, in the manufacture of a medicament for treating cancer in a subject.

In some aspects, the present disclosure provides a use of a pharmaceutical kit comprising at least one compound of the present disclosure, or a pharmaceutically acceptable salt thereof, for treating or preventing cancer in a subject.

In some aspects, the present disclosure provides a use of a pharmaceutical kit comprising a compound of the present disclosure, or a pharmaceutically acceptable salt thereof, for treating or preventing cancer in a subject.

In some aspects, the present disclosure provides a use of a pharmaceutical kit comprising at least one compound of the present disclosure, or a pharmaceutically acceptable salt thereof, for treating cancer in a subject.

In some aspects, the present disclosure provides a use of a pharmaceutical kit comprising a compound of the present disclosure, or a pharmaceutically acceptable salt thereof, for treating cancer in a subject.

BRAF is a human gene located on the long arm of chromosome 7 (7q34) that encodes for a protein known as B-Raf. B-Raf is a serine/threonine kinase that resides in the cytoplasm of cells.

B-Raf is an effector molecule within the mitogen-activated protein kinase (MAPK)/extracellular signal-regulated kinase (ERK) signaling pathway, a pathway that is known to regulate various cellular processes including, but not limited to, growth, proliferation, differentiation and apoptosis.

Briefly, and as would be appreciated by the skilled artisan, in the MAPK/ERK signaling pathway, certain external stimuli, such as growth factors, activate receptors located on the cellular membrane, including receptor tyrosine kinases (RTKs). These receptors in turn activate RAS, causing an exchange of GDP to GTP, thereby producing RAS-GTP. RAS-GTP then activates a Mitogen Activated Protein kinase kinase kinase (MAPKKK or MAP3K). The activated MAPKKK then activates a MAP kinase kinase (MAPKK). The activated MAPKK then activates a MAP kinase (MAPK). Activated MAPK then activates downstream effectors, including transcriptions factors, causing changes in gene expression, thereby regulating the various cellular processes described above, including, but not limited to, cellular growth, proliferation, differentiation and apoptosis.

Examples of MAPKKKs include members of the rapidly accelerated fibrosarcoma (Raf) family, including Raf-1 (also known as C-Raf), B-Raf and A-Raf.

Raf proteins, including B-raf, have three conserved domains denoted conserved region 1 (CR1), conserved region 2 (CR2) and conserved region 3 (CR3). CR1 is an autoinhibitory domain that inhibits the Raf protein's kinase domain (CR3). CR1 includes a binding site for RAS-GTP's effector domain. Upon CR1 binding to RAS-GTP's effector domain, CR1 releases the CR3, relieving autoinhibition of the kinase domain. CR2 is flexible linker that acts as a hinge to connected CR1 and CR3. CR3 is an enzymatic kinase domain.

In its active form, B-Raf forms a dimer and functions as a serine/threonine-specific protein kinase. Under activating conditions, the regulatory protein 14-3-3 is displaced from CR2, of B-Raf, allowing for a de-clamping of CR1 and CR2. Additionally, RAS-GTP binds to CR1 of B-Raf, causing CR1 to release CR3. The overall effect is that the autoinhibition of the kinase domain of B-Raf is relieved. Subsequently, B-Raf is phosphorylated at T599 and S602, which results in the kinase domain switching to the active confirmation. Dimerization can then occur, which further stabilizes the active form of B-Raf.

Mutations in the BRAF gene have been implicated in a variety of different cancers, including, but not limited to, melanoma, non-Hodgkin's lymphoma, colorectal cancer, papillary thyroid carcinoma, non small cell lung cancer (NSCLC) and glioblastoma. As of 2019, approximately 200 BRAF-mutant alleles have been identified in human tumors, with at least 30 distinct mutations having been functionally characterized. BRAF mutations are typically categorized into one of three classes based on the mutations effect on B-Raf activity.

Class I (or Class 1) mutations are mutations that result in the expression of mutant B-Raf that can become active in the monomeric form, independent of RAS activity. That is, Class I mutations in BRAF yield the expression of B-Raf proteins that are RAS-independent, active monomers. These RAS-independent, active monomers typically demonstrate elevated levels of kinase activity.

Class II (or Class 2) mutations are mutations that result in the expression of mutant B-Raf that can form active dimers independent of RAS. That is, Class II mutations in BRAF yield the expression of B-Raf proteins that are RAS-independent, active dimers. These RAS-independent, active dimers also display intermediate to high levels of kinase activity, but their activity levels are typically lower compared to the RAS-independent, active monomers produced by Class I BRAF mutations.

Class III (or Class 3) mutations are mutations that result in the expression of mutant B-Raf that are RAS dependent (i.e. must be activated by RAS-GTP) and that can form heterodimers with other MAPK proteins such as C-Raf. Class III mutations in BRAF typically yield B-Raf with low or impaired kinase activity.

As would be appreciated by the skilled artisan, since Class I BRAF mutations and Class II BRAF mutations are RAS-independent, the mutant B-Raf proteins harboring Class I or Class II mutations are uncoupled to any upstream signals, resulting in constitutive activation that can result in unchecked cellular growth and eventually oncogenic proliferation. In some embodiments, the subject is a mammal.

In some embodiments, the subject is a human.

In some embodiments, the cancer is characterized by at least one oncogenic mutation in the BRAF gene.

It is understood that a cancer that is characterized by at least one oncogenic mutation in the BRAF gene is a cancer that is typically associated with at least one oncogenic mutation in the BRAFgene, including, but not limited to, cancers whose primary oncogenic activity is thought to be driven by the at least one oncogenic mutation in the BRAF gene.

In some embodiments, the cancer is characterized by at least one oncogenic variant of B-Raf.

It is understood that a cancer that is characterized by least one oncogenic variant of B-Raf is a cancer that is typically associated with at least one oncogenic variant of B-Raf, including, but not limited to, cancers whose primary oncogenic activity is thought to be driven by the at least one oncogenic variant of B-Raf.

It is understood that an oncogenic variant of B-Raf is a B-Raf protein that comprises at least one oncogenic mutation and that is produced as the result of the expression of a BRAF gene that comprises at least one oncogenic mutation.

In some embodiments, the subject has at least one oncogenic mutation in the BRAF gene.

In some embodiments, the subject has at least one tumor and/or cancerous cell that expresses an oncogenic variant of B-Raf.

As would be appreciated by the skilled artisan, in the context of a gene (e.g. BRAF), an oncogenic mutation can include, but is not limited to a mutation that results in the substitution of one amino acid for another at a specific position within B-Raf, a mutation that results in the substitution of one or more amino acids for one or more amino acids between two specific positions within B-Raf, a mutation that results in an insertion of one or more amino acids between two positions within B-Raf, a mutation that results in the deletion of one more amino acids between two positions within B-Raf, and mutation that results in a fusion of B-Raf, or portion thereof, with another protein, or portion thereof, or any combination thereof. As would be appreciated by the skilled artisan, in the context of a gene, an oncogenic mutation can include, but is not limited to, a missense mutation, a nonsynonymous mutation, an insertion of one or more nucleotides, a deletion of one or more nucleotides, an inversion and a deletion-insertion. As would be appreciated by the skilled artisan, in the context of a gene (e.g. BRAF), the gene can have one or more of the aforementioned types of oncogenic mutations, including combinations of different types of oncogenic mutations.

As would be appreciated by the skilled artisan, in the context of a protein (e.g. B-Raf), an oncogenic mutation can include, but is not limited to, the substitution of one amino acid for another at a specific position within B-Raf, the substitution of one or more amino acids for one or more amino acids between two specific positions within B-Raf, an insertion of one or more amino acids between two positions within B-Raf, a deletion of one more amino acids between two positions within B-Raf, and a fusion of B-Raf, or portion thereof, with another protein, or portion thereof, or any combination thereof. As would be appreciated by the skilled artisan, in the context of a protein (e.g. B-Raf), the protein can have one or more of the aforementioned types of oncogenic mutations, including combinations of different types of oncogenic mutations.

In some embodiments, an oncogenic mutation of B-Raf can be any of the B-Raf mutations put forth in Table 1a. An oncogenic variant of B-Raf can comprise any combination of the oncogenic mutations put forth in Table 1a. In a non-limiting example, an oncogenic variant of B-Raf can comprise the oncogenic mutations K601E and S363F.

TABLE 1a
B-Raf mutations (numbering corresponding to SEQ ID NO: 1)

| | | | |
|---|---|---|---|
| S363F | G464V | N581S | V600K |
| F247L | G466E | D594G | V600R |
| | G466V | D594N | K601E |
| G464R | S467L | L597Q | K601N |
| L597R | G469A | L597V | |
| G469R | G469V | V600E | L485-P490 Deletion |
| G466A | L584F | K601T | L485-P490 > Y |
| | | | (L485-P490Y) |
| KIAA Fusion | SND-1 Fusion | SKAP2 Fusion | 487-492 Deletion |
| (KIAA1549 | | | |
| fusion) | | | |
| CUL-1 Fusion | | | |

As would be appreciated by the skilled Artisan, L485-P490>Y and L485-P490Y refers to the substitution residues L485 through P490 of B-Raf (SEQ ID NO: 1) with a Tyrosine (Y) residue.

In some embodiments, an oncogenic mutation of B-Raf can comprise a deletion of any combination of one or more amino acids between L485 and P490 of B-Raf (SEQ ID NO: 1). In some embodiments, an oncogenic mutation of B-Raf can comprise a deletion of any combination of one or more amino acids between L485 and Q494 of B-Raf (SEQ ID NO: 1). In some embodiments, an oncogenic mutation of B-Raf can comprise a deletion of any combination of one or more amino acids between A481 and Q494 of B-Raf (SEQ ID NO: 1). In some embodiments, an oncogenic mutation of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

A wild type B-Raf sequence of the present disclosure may comprise, consist essentially of, or consist of the amino acid sequence of:

| (SEQ ID NO: 1) | |
|---|---|
| 1 | MAALSGGGGG GAEPGQALFN GDMEPEAGAG AGAAASSAAD PAIPEEVWNI |
| 51 | KQMIKLTQEH IEALLDKFGG EHNPPSIYLE AYEEYTSKLD ALQQREQQLL |
| 101 | ESLGNGTDFS VSSSASMDTV TSSSSSSLSV LPSSLSVFQN PTDVARSNPK |
| 151 | SPQKPIVRVF LPNKQRTVVP ARCGVTVRDS LKKALMMRGL IPECCAVYRI |
| 201 | QDGEKKPIGW DTDISWLTGE ELHVEVLENV PLTTHNFVRK TFFTLAFCDF |
| 251 | CRKLLFQGFR CQTCGYKFHQ RCSTEVPLMC VNYDQLDLLF VSKFFEHHPI |
| 301 | PQEEASLAET ALTSGSSPSA PASDSIGPQI LTSPSPSKSI PIPQPFRPAD |
| 351 | EDHRNQFGQR DRSSSAPNVH INTIEPVNID DLIRDQGFRG DGGSTTGLSA |
| 401 | TPPASLPGSL TNVKALQKSP GPQRERKSSS SSEDRNRMKT LGRRDSSDDW |
| 451 | EIPDGQITVG QRIGSGSFGT VYKGKWHGDV AVKMLNVTAP TPQQLQAFKN |
| 501 | EVGVLRKTRH VNILLFMGYS TKPQLAIVTQ WCEGSSLYHH LHIIETKFEM |
| 551 | IKLIDIARQT AQGMDYLHAK SIIHRDLKSN NIFLHEDLTV KIGDFGLATV |
| 601 | KSRWSGSHQF EQLSGSILWM APEVIRMQDK NPYSFQSDVY AFGIVLYELM |
| 651 | TGQLPYSNIN NRDQIIFMVG RGYLSPDLSK VRSNCPKAMK RLMAECLKKK |
| 701 | RDERPLFPQI LASIELLARS LPKIHRSASE PSLNRAGFQT EDFSLYACAS |
| 751 | PKTPIQAGGY GAFPVH |

In some embodiments, the oncogenic mutation is a class I mutation. Accordingly, in some embodiments, the oncogenic variant of B-Raf comprises a class I mutation.

In some embodiments, the oncogenic mutation is a class U mutation. Accordingly, in some embodiments, the oncogenic variant of B-Raf comprises a class II mutation.

In some embodiments, the oncogenic mutation is a class III mutation. Accordingly, in some embodiments, the oncogenic variant of B-Raf comprises a class III mutation.

In some embodiments, the oncogenic variant of B-Raf can be any of the B-Raf variants put forth in Table 1b.

TABLE 1b
B-Raf oncogenic variants (numbering corresponding to SEQ ID NO: 1)

| | | | |
|---|---|---|---|
| B-Raf-K601E- | B-Raf-G464V | B-Raf-N581S | B-Raf-V600K |
| S363F | | | |
| B-Raf-F247L | B-Raf-G466E | B-Raf-D594G | B-Raf-V600R |
| | B-Raf-G466V | B-Raf-D594N | B-Raf-K601E |
| B-Raf-G464R | B-Raf-S467L | B-Raf-L597Q | B-Raf-K601N |
| B-Raf-L597R | B-Raf-G469A | B-Raf-L597V | |
| B-Raf-G469R | B-Raf-G469V | B-Raf-V600E | B-RAF-L485- |
| | | | P490 Deletion |
| B-Raf-G466A | B-Raf-L584F | B-Raf-K601T | B-Raf-L485- |
| | | | P490 > Y |
| | | | (B-Raf-L485- |
| | | | P490Y) |
| B-Raf-KIAA | B-Raf-SND-1 | B-Raf-SKAP2 | B-Raf-487- |
| Fusion | Fusion | Fusion | 492 Deletion |
| (B-Raf- | | | |
| KIAA1549 | | | |
| fusion) | | | |
| B-Raf-CUL-1 | | | |
| Fusion | | | |

In some embodiments, an oncogenic variant of B-Raf can comprise a deletion of any combination of one or more amino acids between L485 and P490 of B-Raf (SEQ ID NO: 1). In some embodiments, an oncogenic variant of B-Raf can comprise a deletion of any combination of one or more amino acids between L485 and Q494 of B-Raf (SEQ ID NO: 1). In some embodiments, an oncogenic variant of B-Raf can comprise a deletion of any combination of one or more amino acids between A481 and Q494 of B-Raf (SEQ ID NO: 1). In some embodiments, an oncogenic variant of B-Raf can comprise a deletion of any combination of one or more amino acids between K475 and N500 of B-Raf (SEQ ID NO: 1). In some embodiments, any of the preceding deletions can further comprise any combination of one or more substitutions and/or insertions within the range of residues indicated.

In some embodiments, a subject has at least one tumor and/or cancerous cell that expresses an oncogenic variant of B-Raf and an N-Ras protein comprising at least one mutation. In some as embodiments, an N-Ras protein comprising at least one mutation can be N-Ras-G12D, N-Ras-Q61K, and/or N-Ras-Q61R. In a non-limiting example, a subject can have at least one tumor and/or cancerous cell that expresses B-Raf-D594G and N-Ras-G12D.

In some embodiments, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some embodiments, the cancer is adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocaremoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML). Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer. Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland. Uterine cancer. Vaginal cancer, Vulval cancer, Wilms' tumor, Womb cancer and Gynaecological cancer. Examples of cancer also include, but are not limited to, Hematologic malignancies. Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer. Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some embodiments, the cancer is a hematological cancer.

In some embodiments, the cancer is a solid cancer (also referred to as a solid malignancy or a solid tumor).

In some embodiments, the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

In some embodiments, the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

In some embodiments, the administration does not induce paradoxical activation of wild-type B-Raf.

In some embodiments, the administration does not substantially increase the amount of p-ERK in the subject.

In some embodiments, the administration results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject being administered with vemurafenib or encorafenib.

In some embodiments, the administration results in an amount of p-ERK in the subject that is at least about 10% lower, at least about 20% lower, at least about 30% lower, at least about 40% lower, at least about 50% lower, at least about 60% lower, at least about 70% lower, at least about 80% lower, at least about 90% lower, or at least about 95% lower as compared to a comparable subject without administration.

In some embodiments, the administration reduces the tumor volume in the subject by at least about 10% lower, at least about 20%, at least about 30%, at least about 40%, at least about 50%, at least about 60%, at least about 70%, at least about 80%, at least about 90%, at least about 95%, at least about 98%, or at least about 99%.

### Definitions

Unless otherwise stated, the following terms used in the specification and claims have the following meanings set out below.

As would be appreciated by the skilled artisan, the BRAF gene is commonly referred to as one of BRAF, B-RAF1, BRAF1, NS7, RAFB1, B-Raf proto-oncogene, proto-oncogene B-raf, v-Raf murine sarcoma viral oncogene homolog B, and v-Raf murine sarcoma viral oncogene homolog B1. Thus, these terms are used herein interchangeably to refer to the BRAF gene.

As would be appreciated by the skilled artisan, the B-Raf protein, encoded by the BRAF gene, is commonly referred to as one of BRAF, B-Raf, serine/threonine-protein kinase B-Raf, proto-oncogene B-Raf, p94 and v-Raf murine sarcoma viral oncogene homolog B1. Thus, these terms are used herein interchangeably to refer to the B-Raf gene.

Without wishing to be limited by this statement, it is understood that, while various options for variables are described herein, the disclosure intends to encompass operable embodiments having combinations of the options. The disclosure may be interpreted as excluding the non-operable embodiments caused by certain combinations of the options.

It is to be understood that a compound of the present disclosure may be depicted in a neutral form, a cationic form (e.g., carrying one or more positive charges), or an anionic form (e.g., carrying one or more negative charges), all of which are intended to be included in the scope of the present disclosure. For example, when a compound of the present disclosure is depicted in an anionic form, it should be understood that such depiction also refers to the various neutral forms, cationic forms, and anionic forms of the compound. For another example, when a compound the present disclosure is depicted in an anionic form, it should be understood that such depiction also refers to various salts (e.g., sodium salt) of the anionic form of the compound.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆, for straight chain, C₃-C₆, for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups.

In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydroimidazo[1,2-a]pyridinyl, 6,7,8,9-tetrahydro-5H-imidazo[1,2-a]azepinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidised (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Heteroaryl groups can also be fused or bridged with alicyclic or heterocyclic rings, which are not aromatic so as to form a multicyclic system (e.g., 4,5,6,7-tetrahydrobenzo[c]isoxazolyl).

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthyridine, indole, benzofuran, purine, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encydopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” includes human and non-human animals, as well as cell lines, cell cultures, tissues, and organs. In some embodiments, the subject is a mammal. The mammal can be e.g., a human or appropriate non-human mammal, such as primate, mouse, rat, dog, cat, cow, horse, goat, camel, sheep or a pig. The subject can also be a bird or fowl. In some embodiments, the subject is a human.

As used herein, the term “subject in need thereof” refers to a subject having a disease or having an increased risk of developing the disease. A subject in need thereof can be one who has been previously diagnosed or identified as having a disease or disorder disclosed herein. A subject in need thereof can also be one who is suffering from a disease or disorder disclosed herein.

Alternatively, a subject in need thereof can be one who has an increased risk of developing such disease or disorder relative to the population at large (i.e., a subject who is predisposed to developing such disorder relative to the population at large). A subject in need thereof can have a refractory or resistant a disease or disorder disclosed herein (i.e., a disease or disorder disclosed herein that does not respond or has not yet responded to treatment). The subject may be resistant at start of treatment or may become resistant during treatment. In some embodiments, the subject in need thereof received and failed all known effective therapies for a disease or disorder disclosed herein. In some embodiments, the subject in need thereof received at least one prior therapy.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

It is to be understood that a compound of the present disclosure, or a pharmaceutically acceptable salt, polymorph or solvate thereof, can or may also be used to prevent a relevant disease, condition, or disorder, or used to identify suitable candidates for such purposes.

As used herein, the term “preventing,” “prevent,” or “protecting against” describes reducing or eliminating the onset of the symptoms or complications of such disease, condition or disorder.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., ingestion), inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., a disease or disorder disclosed herein) and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilising processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilisation. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebuliser.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories.

For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease or disorder disclosed herein and also preferably causing complete regression of the disease or disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression.

As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilising the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions.

The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Exemplary Embodiments

Embodiment 1. A compound of Formula (0):

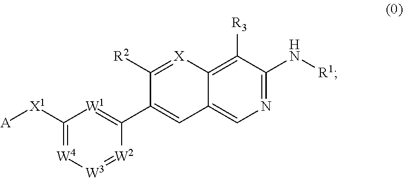

an isomer thereof, or a pharmaceutically acceptable salt thereof, wherein: X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1}; R^{W1} is H, halogen, C₁-C₆ alkyl, C₂-C₆ alkenyl, or C₂-C₆ alkynyl;
   W² is N or CR^{W2}; R² is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R³ is H, halogen, C₁-C₆ alkyl, C₂-C₆ alkenyl, or C₂-C₆ alkynyl;
   W⁴ is N or CR^{W4};
   R⁴ is H, halogen, C₁-C₆ alkyl, C₂-C₆ alkenyl, C₂-C₆ alkynyl, or S(C₁-C₆ alkyl); R¹ is H, CJ-Ce alkyl, C₂-C₆ alkenyl, C₂-C₆ alkynyl, C₁-C₆ alkoxy, C₃-C₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆, alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R³ is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

Embodiment 2. A compound of Formula (I′):

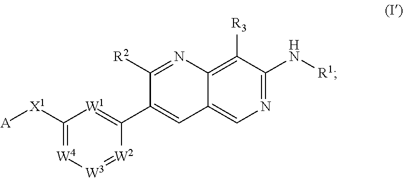

an isomer thereof, or a pharmaceutically acceptable salt thereof, wherein:
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂—C(alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
   R² is H, halogen, cyano, oxo, OH, C₁-C₆ alkyl, C₂-C₆ alkenyl, C₂-C₆ alkynyl, or C₁-C₆ alkoxy;
   R³ is H, halogen, cyano, oxo, OH, C₁-C₆ alkyl, C₂-C₆ alkenyl, C₂-C₆ alkynyl, or C₁-C₆ alkoxy; or
   R¹ and R³, together with the intervening atoms, form a 4- to 12-membered heterocycloalkyl optionally substituted with one or more oxo;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
   A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A}?, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A})₂, C(═O)R^{A1}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

Embodiment 3. A compound of Formula (I):

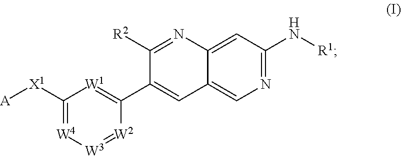

an isomer thereof, or a pharmaceutically acceptable salt thereof, wherein:
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆, alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

Embodiment 4. A compound of Formula (II′):

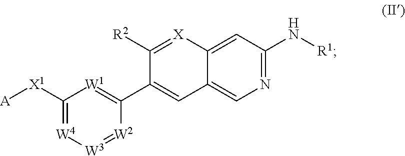

an isomer thereof, or a pharmaceutically acceptable salt thereof, wherein: X is CR^{X} or N;
   R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
   W¹ is N or CR^{W1};
   R^{W1} is H, halogen, C₁-C₆ alkyl, C₂-C₆ alkenyl, or C₂-C₆ alkynyl;
   W² is N or CR^{W2};
   R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
   W³ is N or CR^{W3};
   R^{W3} is H, halogen, C₁-C₆ alkyl, C₂-C₆ alkenyl, or C₂-C₆ alkynyl;
   W⁴ is N or CR^{W4};
   R^{W4} is H, halogen, C₁-C₆ alkyl, C₂-C₆ alkenyl, C₂-C₆ alkynyl, or S(C₁-C₆ alkyl);
   R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
   each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
   R² is H, cyano, oxo, OH, C₁-C₆ alkyl, C₂-C₆ alkenyl, C₂-C₆ alkynyl, or C₁-C₆ alkoxy;
   X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
   R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
   each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
   A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
   each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
   each R^{A1} independently is halogen, cyano, oxo, OH, OR^{A2}, NH₂, NHR^{A2}, N(R^{A2})₂, C(═O)R^{A2}, C₁-C₆, alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
   each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
   wherein R^{A3} is C₁-C₆ alkyl, C₂-C₆ alkenyl, or C₂-C₆ alkynyl.

Embodiment 5. The compound of any one of the preceding embodiments, wherein X is CR^{X}.

Embodiment 6. The compound of any one of the preceding embodiments, wherein X is N.

Embodiment 7. The compound of any one of the preceding embodiments, wherein:
W¹ is CR^{W1};
R^{W1} is H, halogen, or C₁-C₆ alkyl;
W² is N or CR^{W2}—;
R^{W2} is H, halogen, or C₁-C₆ alkyl;
W³ is N or CR^{W3};
R^{W3} is H, or halogen;
W⁴ is N or CR^{W4};
R^{W4} is H, or halogen;
R¹ is H, or C₁-C₆ alkyl, wherein the C₁-C₆ alkyl is optionally substituted with one or more R^{1a};
each R^{1a} is cyano;
R² is H, or cyano;
X¹ is —NR^{X1}—*, —C(═O)NR^{X1}—*, —NR^{X1}C(═O)—*, or —NR^{X1}C(═NH)—*, wherein * denotes attachment to A;
R^{X1} is H;
A is C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl), wherein the C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl) is optionally substituted with one or more R^{A};
each R^{A} independently is halogen, OH, OR^{A1}, NHR^{1A}, N(R^{A1})₂, C₁-C₆ alkyl, C₁-C₆ alkoxy, or C₃-C₈ cycloalkyl, wherein the C₁-C₆ alkyl, C₁-C₆ alkoxy, or C₃-C₈ cycloalkyl is optionally substituted with one or more R^{A1};
each R^{A1} independently is halogen, cyano, oxo, OH, OR^{A2}, NH₂, or C₁-C₆ alkyl, wherein the C₁-C₆ alkyl is optionally substituted with one or more R^{A2}; and
each R^{A2} independently is halogen, OH, or C₁-C₆ alkyl.

Embodiment 8. The compound of any one of the preceding embodiments, wherein W¹ is CR^{W1}.

Embodiment 9. The compound of any one of the preceding embodiments, wherein R^{W1} is H.

Embodiment 10. The compound of any one of the preceding embodiments, wherein R^{W1} is halogen.

Embodiment 11. The compound of any one of the preceding embodiments, wherein W¹ is CH.

Embodiment 12. The compound of any one of the preceding embodiments, wherein W¹ is N.

Embodiment 13. The compound of any one of the preceding embodiments, wherein R^{W1} is C₁-C₆ alkyl.

Embodiment 14. The compound of any one of the preceding embodiments, wherein R^{W1} is halogen, C₁-C₆ alkyl, C₂-C₆ alkenyl, or C₂-C₆ alkynyl.

Embodiment 15. The compound of any one of the preceding embodiments, wherein W² is N.

Embodiment 16. The compound of any one of the preceding embodiments, wherein W² is CR^{W2}.

Embodiment 17. The compound of any one of the preceding embodiments, wherein R^{W2} is H.

Embodiment 18. The compound of any one of the preceding embodiments, wherein R^{W2} is halogen.

Embodiment 19. The compound of any one of the preceding embodiments, wherein W² is CH.

Embodiment 20. The compound of any one of the preceding embodiments, R^{W2} is C₁-C₆ alkyl.

Embodiment 21. The compound of any one of the preceding embodiments, wherein W² is C(CH₃).

Embodiment 22. The compound of any one of the preceding embodiments, wherein R^{W2} is halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen.

Embodiment 23. The compound of any one of the preceding embodiments, wherein W¹ is CH, W² is C(CH₃).

Embodiment 24. The compound of any one of the preceding embodiments, wherein W³ is CR^{W3}.

Embodiment 25. The compound of any one of the preceding embodiments, wherein R^{W3} is H.

Embodiment 26. The compound of any one of the preceding embodiments, wherein W³ is CH.

Embodiment 27. The compound of any one of the preceding embodiments, wherein W³ is N.

Embodiment 28. The compound of any one of the preceding embodiments, wherein R^{W3} is halogen.

Embodiment 29. The compound of any one of the preceding embodiments, wherein R^{W3} is C₁-C₆ alkyl, C₂-C₆ alkenyl, or C₂-C₆ alkynyl.

Embodiment 30. The compound of any one of the preceding embodiments, wherein W⁴ is CR^{W4}.

Embodiment 31. The compound of any one of the preceding embodiments, wherein R^{W4} is H.

Embodiment 32. The compound of any one of the preceding embodiments, wherein W⁴ is CH.

Embodiment 33. The compound of any one of the preceding embodiments, wherein W⁴ is N.

Embodiment 34. The compound of any one of the preceding embodiments, wherein R^{W4} is halogen.

Embodiment 35. The compound of any one of the preceding embodiments, wherein R^{W4} is C₁-C₆ alkyl, C₂-C₆ alkenyl, C₂-C₆ alkynyl, or S(C₁-C₆ alkyl).

Embodiment 36. The compound of any one of the preceding embodiments, wherein R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more Ra.

Embodiment 37. The compound of any one of the preceding embodiments, wherein R¹ is H.

Embodiment 38. The compound of any one of the preceding embodiments, wherein R¹ is C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more R^{1a}.

Embodiment 39. The compound of any one of the preceding embodiments, wherein R¹ is C₁-C₆ alkyl optionally substituted with one or more R^{1a}.

Embodiment 40. The compound of any one of the preceding embodiments, wherein R¹ is CH₃.

Embodiment 41. The compound of any one of the preceding embodiments, wherein R¹ is CH₂CH₃.

Embodiment 42. The compound of any one of the preceding embodiments, wherein R^{1a} is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl.

Embodiment 43. The compound of any one of the preceding embodiments, wherein R^{1a} is cyano.

Embodiment 44. The compound of any one of the preceding embodiments, wherein R² is H, cyano, oxo, OH, C₁-C₆ alkyl, C₂-C₆ alkenyl, C₂-C₆ alkynyl, or C₁-C₆ alkoxy.

Embodiment 45. The compound of any one of the preceding embodiments, wherein R² is H or cyano.

Embodiment 46. The compound of any one of the preceding embodiments, wherein R² is H.

Embodiment 47. The compound of any one of the preceding embodiments, wherein R² is cyano.

Embodiment 48. The compound of any one of the preceding embodiments, wherein X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A.

Embodiment 49. The compound of any one of the preceding embodiments, wherein X¹ is —NR^{X1}—*, wherein * denotes attachment to A.

Embodiment 50. The compound of any one of the preceding embodiments, wherein X¹ is —NH—*, wherein * denotes attachment to A.

Embodiment 51. The compound of any one of the preceding embodiments, wherein X¹ is —NR^{X1}C(═O)—*, wherein * denotes attachment to A.

Embodiment 52. The compound of any one of the preceding embodiments, wherein X¹ is —NHC(═O)—*, wherein * denotes attachment to A.

Embodiment 53. The compound of any one of the preceding embodiments, wherein X¹ is —C(═O)NR^{X1}—*, wherein * denotes attachment to A.

Embodiment 54. The compound of any one of the preceding embodiments, wherein X¹ is —C(═O)NH—*, wherein * denotes attachment to A.

Embodiment 55. The compound of any one of the preceding embodiments, wherein X¹ is —NR^{X1}C(═NH)—*, wherein * denotes attachment to A.

Embodiment 56. The compound of any one of the preceding embodiments, wherein X¹ is —NHC(═NH)—*, wherein * denotes attachment to A.

Embodiment 57. The compound of any one of the preceding embodiments, wherein R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁—C alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a}.

Embodiment 58. The compound of any one of the preceding embodiments, wherein R^{X1} is H.

Embodiment 59. The compound of any one of the preceding embodiments, wherein R^{X1} is C₁-C₆ alkyl, optionally substituted with one or more R^{X1a}.

Embodiment 60. The compound of any one of the preceding embodiments, wherein R^{X1} is CH₃.

Embodiment 61. The compound of any one of the preceding embodiments, wherein R^{X1a} is halogen, C₁-C₆ alkyl, or 3- to 8-membered heterocycloalkyl, wherein the C₁-C₆ alkyl, or 3- to 8-membered heterocycloalkyl is optionally substituted with one or more halogen.

Embodiment 62. The compound of any one of the preceding embodiments, wherein A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A}.

Embodiment 63. The compound of any one of the preceding embodiments, wherein A is C₆-C₁₀ aryl, optionally substituted with one or more R^{A}.

Embodiment 64. The compound of any one of the preceding embodiments, wherein A is phenyl optionally substituted with one or more R^{A}.

Embodiment 65. The compound of any one of the preceding embodiments, wherein A is 5- to 10-membered heteroaryl, optionally substituted with one or more R^{A}.

Embodiment 66. The compound of any one of the preceding embodiments, wherein A is pyridyl optionally substituted with one or more R^{A}.

Embodiment 67. The compound of any one of the preceding embodiments, wherein A is pyrazolyl optionally substituted with one or more R^{A}.

Embodiment 68. The compound of any one of the preceding embodiments, wherein A is imidazolyl optionally substituted with one or more R^{A}.

Embodiment 69. The compound of any one of the preceding embodiments, wherein A is oxazolyl optionally substituted with one or more R^{A}.

Embodiment 70. The compound of any one of the preceding embodiments, wherein A is imidazo[1,5-a]pyridyl optionally substituted with one or more R^{A}.

Embodiment 71. The compound of any one of the preceding embodiments, wherein A is 2,3-dihydrofuro[2,3-c]pyridyl optionally substituted with one or more R^{A}.

Embodiment 72. The compound of any one of the preceding embodiments, wherein A is 2,3-dihydrofuro[3,2-b]pyridyl optionally substituted with one or more R^{A}.

Embodiment 73. The compound of any one of the preceding embodiments, wherein A is 3,4-dihydro-1H-pyrano[3,4-c]pyridyl optionally substituted with one or more R^{A}.

Embodiment 74. The compound of any one of the preceding embodiments, wherein A is 4,5,6,7-tetrahydrobenzo[d]isoxazolyl optionally substituted with one or more R^{A}.

Embodiment 75. The compound of any of the preceding embodiments, wherein A is —(C₁-C₆ alkyl)-(5- to 10-membered heteroaryl), optionally substituted with one or more R^{A}.

Embodiment 76. The compound of any of the preceding embodiments, wherein A is —(C₂ alkyl)-(triazolyl) optionally substituted with one or more R^{A}.

Embodiment 77. The compound of any of the preceding embodiments, wherein A is —(C₁-C₆ alkyl)-(3- to 8-membered heterocycloalkyl), optionally substituted with one or more R^{A}.

Embodiment 78. The compound of any of the preceding embodiments, wherein A is —(C₁ alkyl)-(piperidinyl) optionally substituted with one or more R^{A}.

Embodiment 79. The compound of any of the preceding embodiments, wherein A is —(C₁ alkyl)-(tetrahydropyranyl) optionally substituted with one or more R^{A}.

Embodiment 80. The compound of any of the preceding embodiments, wherein X¹ is —NHC(═O)—*, wherein * denotes attachment to A, and A is pyridyl, optionally substituted with one or more R^{A}.

Embodiment 81. The compound of any of the preceding embodiments, wherein R^{A} is halogen, cyano, oxo, OH, OR^{A1}, NH₂, NHR^{A1}, N(R^{A1})₂, (═N)R^{A}, C₁-C₆ alkyl, C₂-C₆ alkenyl, C₂-C₆alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1}.

Embodiment 82. The compound of any of the preceding embodiments, wherein R^{A} is halogen.

Embodiment 83. The compound of any of the preceding embodiments, wherein R^{A} is cyano.

Embodiment 84. The compound of any of the preceding embodiments, wherein R^{A} is OH.

Embodiment 85. The compound of any of the preceding embodiments, wherein R^{A} is O(R^{A1}).

Embodiment 86. The compound of any of the preceding embodiments, wherein R^{A} is NHR^{A1}.

Embodiment 87. The compound of any of the preceding embodiments, wherein R^{A} is N(R^{A})₂.

Embodiment 88. The compound of any of the preceding embodiments, wherein R^{A} is C₁-C₆ alkyl, optionally substituted with one or more R^{A1}.

Embodiment 89. The compound of any of the preceding embodiments, wherein R^{A} is C₁ alkyl, optionally substituted with one or more R^{A1}.

Embodiment 90. The compound of any of the preceding embodiments, wherein R^{A} is C₃ alkyl, optionally substituted with one or more R^{A1}.

Embodiment 91. The compound of any of the preceding embodiments, wherein R^{A} is C₁-C₆ alkoxy, optionally substituted with one or more R^{A1}.

Embodiment 92. The compound of any of the preceding embodiments, wherein R^{A} is CH₃.

Embodiment 93. The compound of any of the preceding embodiments, wherein R^{A} is CF₃.

Embodiment 94. The compound of any of the preceding embodiments, wherein R^{A} is C(CH₃)₂CN.

Embodiment 95. The compound of any of the preceding embodiments, wherein R^{A} is C₃-C₈ cycloalkyl, optionally substituted with one or more R^{A1}.

Embodiment 96. The compound of any of the preceding embodiments, wherein R^{A} is C₃ cycloalkyl, optionally substituted with one or more R^{A}.

Embodiment 97. The compound of any of the preceding embodiments, wherein R^{A} is C₄ cycloalkyl, optionally substituted with one or more R^{A1}.

Embodiment 98. The compound of any of the preceding embodiments, wherein R^{A1} is halogen, cyano, oxo, OH, OR^{A2}, NH₂, NHR^{A2}, N(R^{A2})₂, C(═O)R^{A2}, C₁-C₆, alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}.

Embodiment 99. The compound of any of the preceding embodiments, wherein R^{A1} is halogen.

Embodiment 100. The compound of any of the preceding embodiments, wherein R^{A1} is fluorine.

Embodiment 101. The compound of any of the preceding embodiments, wherein R^{A1} is cyano.

Embodiment 102. The compound of any of the preceding embodiments, wherein R^{A1} is oxo.

Embodiment 103. The compound of any of the preceding embodiments, wherein R^{A1} is OH.

Embodiment 104. The compound of any of the preceding embodiments, wherein R^{A1} is OR^{A2}.

Embodiment 105. The compound of any of the preceding embodiments, wherein R^{A1} is NH₂.

Embodiment 106. The compound of any of the preceding embodiments, wherein R^{A1} is C₁-C₆ alkyl, optionally substituted with one or more R^{A2}.

Embodiment 107. The compound of any of the preceding embodiments, wherein R^{A2} is halogen, cyano, OH, NH₂, N(R^{A1})₂, C(═O)R^{A3}, C₁-C₆ alkyl, C₂-C₆ alkenyl, or C₂-C₆ alkynyl.

Embodiment 108. The compound of any of the preceding embodiments, wherein R^{A2} is halogen.

Embodiment 109. The compound of any of the preceding embodiments, wherein R^{A2} is OH.

Embodiment 110. The compound of any of the preceding embodiments, wherein R^{A2} is C₁-C₆ alkyl.

Embodiment 111. The compound of any of the preceding embodiments, wherein R^{A2} is C₁-C₆ alkyl, C₂-C₆ alkenyl, or C₂-C₆ alkynyl.

Embodiment 112. The compound of any of the preceding embodiments, wherein the compound is of Formula (I-a), (I-b), (I-c), (I-d), (I-e), or (I-f):

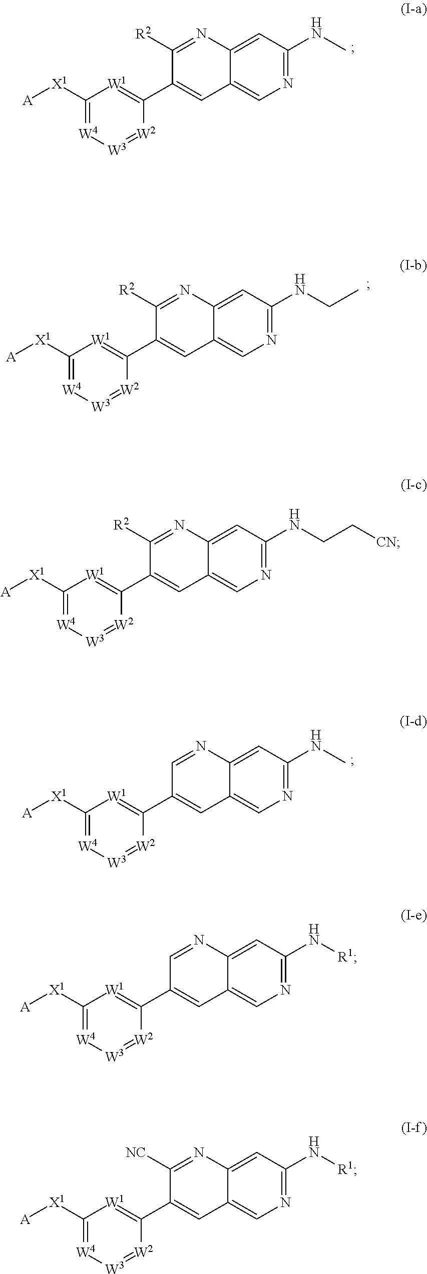

or a pharmaceutically acceptable salt or stereoisomer thereof.

Embodiment 113. The compound of any of the preceding embodiments, wherein the compound is of Formula (I-g), (I-h), (I-i), (I-j), (I-k), (I-l), (I-m), (I-n), (I-o), (I-p), (I-q), (I-r), (I-s), or (I-t):

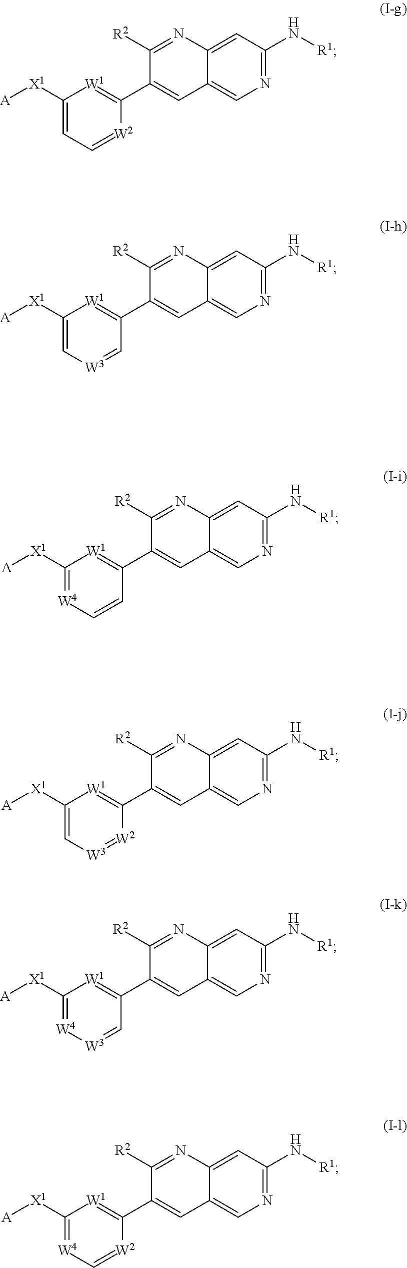

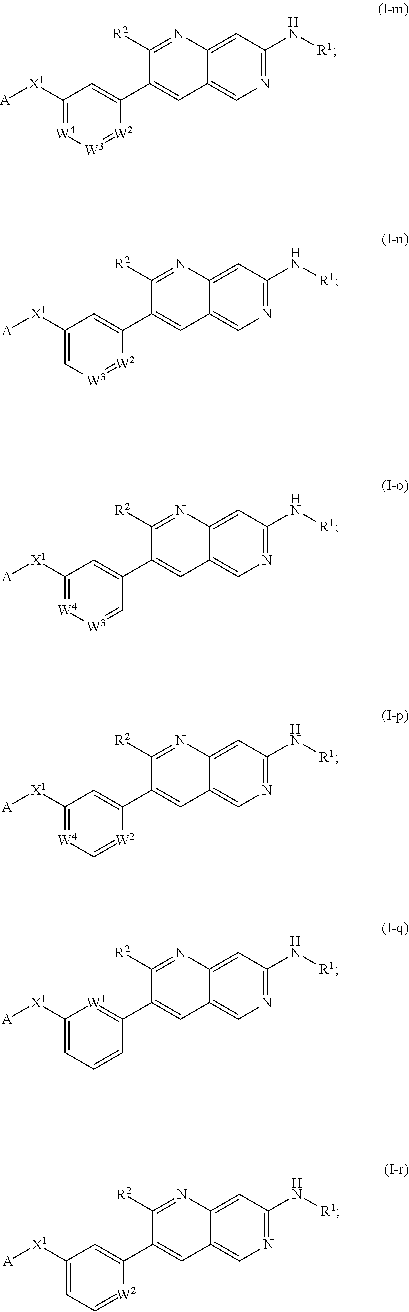

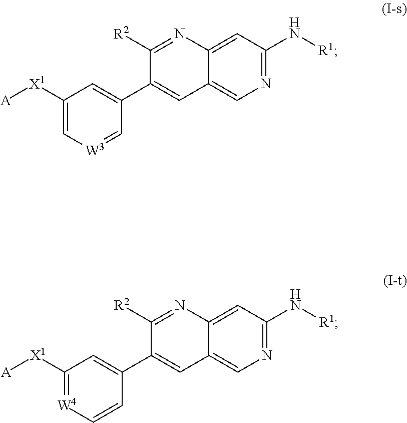

or a pharmaceutically acceptable salt or stereoisomer thereof.

Embodiment 114. The compound of any of the preceding embodiments, wherein the compound is of Formula (I-u), (I-v), (I-w), or (I-x).

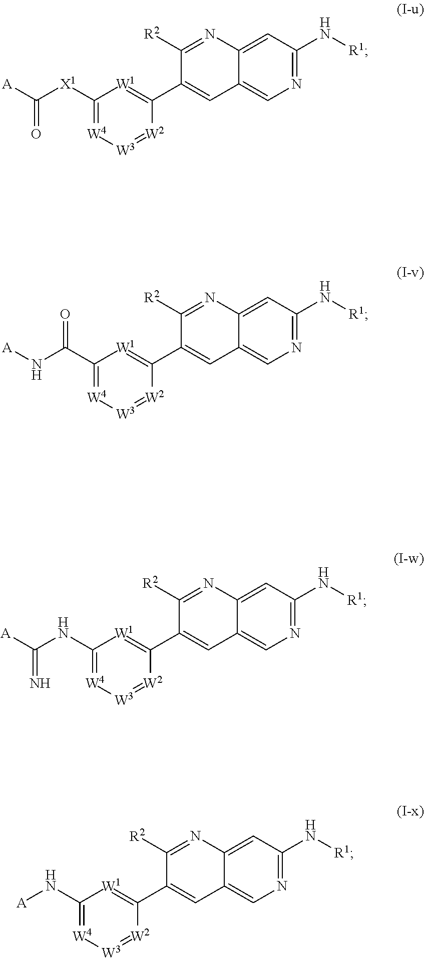

or a pharmaceutically acceptable salt or stereoisomer thereof.

Embodiment 115. The compound of any of the preceding embodiments, wherein the compound is selected from the compounds described in Table I or Table II, or a pharmaceutically acceptable salt or stereoisomer thereof.

Embodiment 116. The compound of any of the preceding embodiments, wherein the compound is selected from the compounds described in Table I, or a pharmaceutically acceptable salt or stereoisomer thereof.

Embodiment 117. The compound of any of the preceding embodiments, wherein the compound is selected from the compounds described in Table II, or a pharmaceutically acceptable salt or stereoisomer thereof.

Embodiment 118. An isotopic derivative of the compound of any one of the preceding embodiments.

Embodiment 119. A method of preparing the compound of any one of the preceding embodiments.

Embodiment 120. A pharmaceutical composition comprising the compound of any one of the preceding embodiments and one or more pharmaceutically acceptable carriers or excipients.

Embodiment 121. A method of treating or preventing cancer in a subject, the method comprising administering to the subject a therapeutically effective amount of the compound of any one of the preceding embodiments.

Embodiment 122. The compound of any one of the preceding embodiments for treating or preventing cancer in a subject.

Embodiment 123. Use of the compound of any one of the preceding embodiments in the manufacture of a medicament for treating or preventing cancer in a subject.

Embodiment 124. The method, compound, or use of any one of the preceding embodiments, wherein the subject is a human.

Embodiment 125. The method, compound, or use of any one of the preceding embodiments, wherein the cancer is characterized by at least one oncogenic mutation in the BRAF gene.

Embodiment 126. The method, compound, or use of any one of the preceding embodiments, wherein the cancer is characterized by at least one oncogenic variant of B-Raf.

Embodiment 127. The method, compound, or use of any one of the preceding embodiments, wherein the subject has at least one oncogenic mutation in the BRAFgene.

Embodiment 128. The method, compound, or use of any one of the preceding embodiments, wherein the subject has at least one tumor and/or cancerous cell that expresses an oncogenic variant of B-Raf.

Embodiment 129. The method, compound, or use of any one of the preceding embodiments, wherein the oncogenic mutation is a class I mutation. Accordingly, in some embodiments, the oncogenic variant of B-Raf comprises a class I mutation.

Embodiment 130. The method, compound, or use of any one of the preceding embodiments, wherein the oncogenic mutation is a class II mutation. Accordingly, in some embodiments, the oncogenic variant of B-Raf comprises a class II mutation.

Embodiment 131. The method, compound, or use of any one of the preceding embodiments, wherein the oncogenic mutation is a class III mutation.

Embodiment 132. The method, compound, or use of any one of the preceding embodiments, wherein the oncogenic variant of B-Raf can be any of the B-Raf variants put forth in Table 1b.

Embodiment 133. The method, compound, or use of any one of the preceding embodiments, wherein the cancer is a hematological cancer.

Embodiment 134. The method, compound, or use of any one of the preceding embodiments, wherein the cancer is a solid cancer.

Embodiment 135. The method, compound, or use of any one of the preceding embodiments, wherein the cancer is melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, or thyroid cancer.

Embodiment 136. The method, compound, or use of any one of the preceding embodiments, wherein the cancer is non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.

### EXAMPLES

For exemplary purpose, neutral compounds of Formula (0), (I′), (I) and (II′) are synthesized and tested in the examples. It is understood that the neutral compounds of Formula (0), (I′), (I) and (II′) may be converted to the corresponding pharmaceutically acceptable salts of the compounds using routine techniques in the art (e.g., by saponification of an ester to the carboxylic acid salt, or by hydrolyzing an amide to form a corresponding carboxylic acid and then converting the carboxylic acid to a carboxylic acid salt).

Compounds of Formula (0), (I′), (I) and (II′) can be prepared using the methods detailed herein. Those skilled in the art may be able to envisage alternative synthetic routes, using a variety of starting materials and reagents to prepare the disclosed compounds of Formula (0), (I′), (I) and (II′) and to make further modifications. For exemplary purpose, salts of some of the compounds of Formula (0), (I′), (I) and (II′) are synthesized and tested in the examples. It is understood that neutral compounds of Formula (0), (I′), (I) and (II′) may be similarly synthesized and tested using the exemplary procedures described in the examples. Further, it is understood that the salts (e.g., hydrochloride salt) of the compounds of Formula (0), (I′), (I) and (II′) may be converted to the corresponding neutral compounds using routine techniques in the art (e.g., pH adjustment and, optionally, extraction (e.g., into an aqueous phase)).

### Abbreviations

¹H NMR Proton nuclear magnetic resonance spectroscopy
ACN Acetonitrile
aq. Aqueous
BPO Benzoyl peroxide
BuLi Butyllithium
CbzCl Benzyl chloroformate
CDCl₃ Deuterated chloroform
DAST Diethylaminosulphur trifluoride
DCE Dichloroethane
DCM Dichloromethane
DHP 3,4-Dihydro-2H-pyran
DIEA N,N-Diisopropylethylamine
DIPA Diisopropylamine
DIPEA N,N-Diisopropylethylamine
DMA Dimethylacetamide
DMAP 4-Dimethylaminopyridine
DME 1,2-Dimethoxyethane
DMF Dimethylformamide
DMPU 1,3-Dimethyl-3,4,5,6-tetrahydro-2(1H)-pyrimidinone
DMSO Dimethyl sulfoxide
DMSO-d₆ Hexadeuterodimethylsulfoxide
eq. Equivalents
Et₃N Triethylamine
Et₃SiH Triethylsilane
EtOAc Ethyl acetate
EtOH Ethanol
h Hour(s)
HATU 1-[bis(dimethylamino)methylene]-1H-1,2,3-triazolo[4,5-b]pyridinium 3-oxid
hexafluorophosphate
HPLC High performance liquid chromatography
LAH Lithium aluminum hydride
LC-MS Liquid chromatography-mass spectrometry
LDA Lithium diisopropylamide
LiHMDS Lithium hexamethyldisilazide
mCPBA 3-Chloroperbenzoic acid
M Molar
MeCN Acetonitrile
MeOH Methanol
min Minute(s)
MsCl Methanesulfonyl chloride
N Normality
NBS N-Bromosuccinimide
NCS N-Chlorosuccinimide
NIS N-Iodosuccinimide
NMP 1-Methyl-2-pyrrolidinone
prep-HPLC preparative high performance liquid chromatography
Py Pyridine
rt room temperature
SEMCl 2-(trimethylsilyl)ethoxymethyl chloride
SFC Supercritical fluid chromatography
TBAF Tetrabutylammonium fluoride
TBTU 2-(1H-benzotriazole-1-yl)-1,1,3,3-tetramethylaminium tetrafluoroborate
TEA Triethylamine
TFA Trifluoroacetic acid
TFAA Trifluoroacetic anhydride
THE Tetrahydrofuran
TLC Thin layer chromatography
TMP 2,2,6,6-Tetramethylpiperidine
TMSCF3 Trimethyl(trifluoromethyl)silane
TMSCl Chlorotrimethylsilane
TMSCN Trimethylsilyl cyanide
TsCl Tosyl chloride
TsOH 4-Methylbenzene sulfonic acid
UHP Urea hydrogen peroxide
Y Yield

### SYNTHESIS OF INTERMEDIATES

### Intermediate 1. Synthesis of 3-bromo-N-(4-methoxybenzyl)-N-methyl-1,6-naphthyridin-7-amine

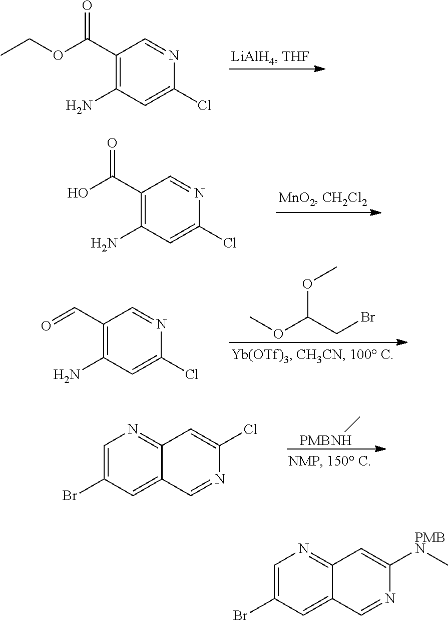

Step 1. Lithium aluminum hydride (6 mL, 6.0 mmol) was added in portions to a solution of ethyl 4-amino-6-chloronicotinate (1.0 g, 4.985 mmol) at 0° C. under nitrogen. The mixture was stirred at 25° C. for 1 hour. The reaction was quenched with sodium sulfate decahydrate (5.0 g), filtered through celite, and washed with dichloromethane (300 mL). The filtrate was concentrated to afford (4-amino-6-chloropyridin-3-yl)methanol (830 mg, 5.25 mmol, 87%) as a brown oil, which was used without further purification. MS (ESI) m/z 159.0 [M+H]⁺

Step 2. To a solution of (4-amino-6-chloropyridin-3-yl)methanol (830 mg, 5.25 mmol) in dichloromethane (10 mL) was added manganese dioxide (4.6 g, 52.3 mmol) at 25° C. After stirring at 25° C. for 12 h, the reaction mixture was filtered through celite and the filtrate was concentrated. The resulting residue was purified by flash chromatography (silica, petroleum ether/ethyl acetate=2/1) to afford 4-amino-6-chloronicotinaldehyde (560 mg, 3.59 mmol, 68%) as a white solid. MS (ESI) m/z 156.9 [M+H]⁺

Step 3. To a mixture of 4-amino-6-chloronicotinaldehyde (0.4 g, 2.88 mmol) and ytterbium(III) trifluoromethanesulfonate (0.36 g, 0.57 mmol) in acetonitrile (8 mL) was added 2-bromo-1,1-dimethoxyethane (1.0 ml, 8.65 mmol) at 0° C. under nitrogen. The mixture was stirred at 100° C. for 16 hours. The reaction was quenched with water (100 mL) and extracted with dichloromethane (100 mL×2). The combined organic layer was washed with brine (100 mL), dried over anhydrous sodium sulfate, filtered, and concentrated. The residue was purified by flash chromatography (silica, petroleum ether/ethyl acetate=7/1) to afford 3-bromo-7-chloro-1,6-naphthyridine (270 mg, 1.12 mmol, 38%) as a yellow solid. MS (ESI) m/z 242.9 [M+H]⁺

Step 4. To a solution of 3-bromo-7-chloro-1,6-naphthyridine (270 mg, 1.12 mmol) in N-methyl pyrrolidone (6 mL) was added (4-methoxyphenyl)methanamine (505 mg, 3.33 mmol). The mixture was stirred at 150° C. for 6 hours. After cooling to room temperature, the reaction was quenched with water (50 mL) and extracted with dichloromethane (80 mL). The organic layer was washed with brine (80 mL), dried over anhydrous sodium sulfate, filtered, and concentrated. The residue was purified by flash chromatography (silica, petroleum ether/ethyl acetate=5/2) to afford-3-bromo-N-(4-methoxybenzyl)-N-methyl-1,6-naphthyridin-7-amine (180 mg, 0.503 mmol, 45%) as a yellow solid. MS (ESI) m/z 357.9 [M+H]⁺

### Intermediate 2. Synthesis of 3-(5-amino-2-methylphenyl)-N-(4-methoxybenzyl)-N-methyl-1,6-naphthyridin-7-amine

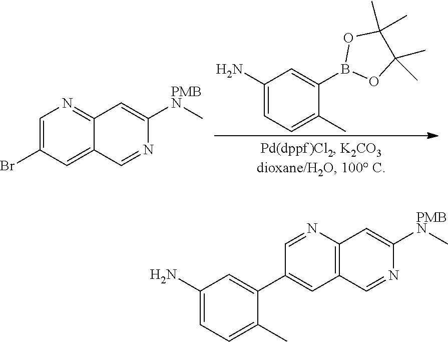

Step 5. A mixture of 3-bromo-N-(4-methoxybenzyl)-N-methyl-1,6-naphthyridin-7-amine (180 mg, 0.502 mmol), 4-methyl-3-(4,4,5,5-tetramethyl-1,3,2-dioxaborolan-2-yl)aniline (164 mg, 0.704 mmol), 1,1′-bis(diphenylphosphino)ferrocene-palladium(II)dichloride (370 mg, 0.0502 mmol) and potassium carbonate (208 mg, 1.506 mmol) in water (2 mL) and dioxane (10 mL) was stirred at 100° C. for 2 hours under argon. The reaction mixture was cooled to room temperature. The organic layer was concentrated and purified by flash chromatography (silica, dichloromethanelmethanol=100/6) to afford 3-(5-amino-2-methylphenyl)-N-(4-methoxybenzyl)-N-methyl-1,6-naphthyridin-7-amine (120 mg, 0.313 mmol, 62%) as a yellow oil. MS (ESI) m/z 385.2 [M+H]⁺

### Intermediate 3. Synthesis of 3-(5-amino-2-methylphenyl)-N-methyl-1,6-naphthyridin-7-amine

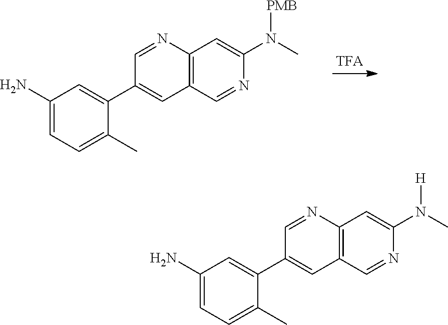

A mixture of 3-(5-amino-2-methylphenyl)-N-(4-methoxybenzyl)-N-methyl-1,6-naphthyridin-7-amine (600 mg, 1.56 mmol) in trifluoroacetic acid (5 mL) and dichloromethane (5 mL) was stirred at room temperature for 1 hour. The resulting mixture was concentrated under reduced pressure, and the residue was quenched with saturated sodium bicarbonate solution and extracted with ethyl acetate (30 mL×3). The combined organic phases were washed with water, dried over sodium sulfate, filtered, concentrated, and purified by flash chromatography (silica, 5% methanol in dichloromethane) to afford 3-(5-amino-2-methylphenyl)-N-methyl-1,6-naphthyridin-7-amine (350 mg, 1.32 mmol, 85%) as a pale yellow solid. MS (ESI) m/z 265.2 [M+H]⁺

### Intermediate 4. Synthesis of 3-(5-amino-2-methylpyridin-3-yl)-N-(4-methoxybenzyl)-N-methyl-1,6-naphthyridin-7-amine

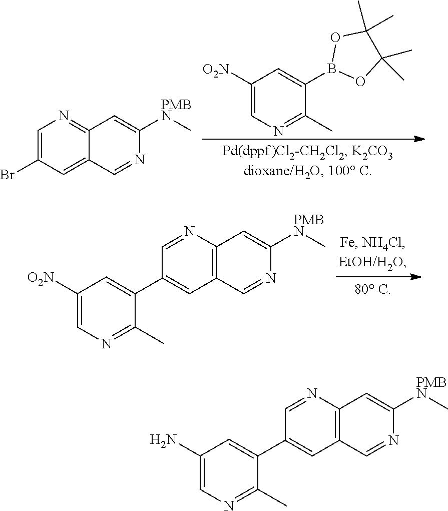

Step 1. A mixture of 3-bromo-N-(4-methoxybenzyl)-N-methyl-1,6-naphthyridin-7-amine (250 mg, 0.7 mmol), 2-methyl-5-nitro-3-(4,4,5,5-tetramethyl-1,3,2-dioxaborolan-2-yl)pyridine (185 mg, 0.7 mmol), 1,1′-bis(diphenylphosphino)ferrocene-palladium(II)dichloride dichloromethane complex (57 mg, 0.07 mmol) and potassium carbonate (193 mg, 1.4 mmol) in 1,4-dioxane/water (5 mL) was stirred at 100° C. under argon atmosphere for 2 hours. The reaction mixture was concentrated under vacuum. The residue was purified by flash chromatography (silica, petroleum ether/ethyl acetate=1/1) to afford N-(4-methoxybenzyl)-N-methyl-3-(2-methyl-5-nitropyridin-3-yl)-1,6-naphthyridin-7-amine (250 mg, 0.60 mmol, 86%) as a yellow oil. MS (ESI) m/z 416.0 [M+H]⁺

Step 2. A mixture of N-(4-methoxybenzyl)-N-methyl-3-(2-methyl-5-nitropyridin-3-yl)-1,6-naphthyridin-7-amine (250 mg, 0.60 mmol), iron powder (135 mg, 2.4 mmol) and ammonium chloride (257 mg, 4.8 mmol) in ethanol/water=5/1 (6 mL) was stirred at 80° C. under argon atmosphere for 2 hours. The mixture was cooled to room temperature and filtered. The filtrate was diluted with water (20 mL), then extracted with ethyl acetate (20 mL×2). The combined organic layers were washed with brine (20 mL), dried over anhydrous sodium sulfate, filtered, and concentrated to afford 3-(5-amino-2-methylpyridin-3-yl)-N-(4-methoxybenzyl)-N-methyl-1,6-naphthyridin-7-amine (215 mg, 0.56 mmol, 93%) as a yellow solid. MS (ESI) m/z 386.1 [M+H]⁺

### SYNTHESIS OF EXAMPLES

### Example 1. Synthesis of N-(4-methyl-3-(7-(methylamino)-1,6-naphthyridin-3-yl)phenyl)-4-(trifluoromethyl)picolinamide (Compound 1)

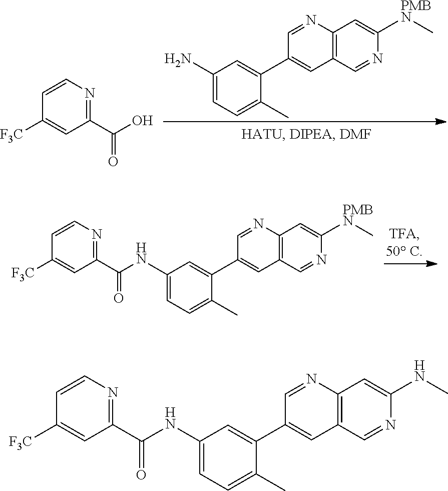

Step 1. A solution of 4-(trifluoromethyl)picolinic acid (30 mg, 0.16 mmol), N,N-diisopropylethylamine (60.7 mg, 0.47 mmol), 1-[bis(dimethylamino)methylene]-1H-1,2,3-triazolo[4,5-b]pyridinium 3-oxid hexafluorophosphate (179 mg, 0.32 mmol) and 3-(5-amino-2-methylphenyl)-N-(4-methoxybenzyl)-N-methyl-1,6-naphthyridin-7-amine (44.5 mg, 0.08 mmol) in N,N-dimethylformamide (3 mL) was stirred at room temperature for 2 hours. The mixture was diluted with water (10 mL), and then extracted with ethyl acetate (20 mL×3). The combined organic layer was washed with brine (10 mL), dried over anhydrous sodium sulfate, and filtered. The filtrate was concentrated under reduced pressure to afford N-(3-(7-((4-methoxybenzyl)(methyl)amino)-1,6-naphthyridin-3-yl)-4-methylphenyl)-4-(trifluoromethyl)picolinamide (45 mg, crude) as a yellow solid. MS (ESI) m/z 558.3 [M+H]⁺

Step 2. A mixture of N-(3-(7-((4-methoxybenzyl)(methyl)amino)-1,6-naphthyridin-3-yl)-4-methylphenyl)-4-(trifluoromethyl)picolinamide (45 mg, crude) in trifluoroacetic acid (5 mL) was stirred at 50° C. for 4 hours. After cooling to room temperature, the reaction was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-methyl-3-(7-(methylamino)-1,6-naphthyridin-3-yl)phenyl)-4-(trifluoromethyl)picolinamide (23.3 mg, 0.053 mmol) as a yellow solid. ¹H NMR (400 MHz, DMSO-d₆) δ 10.87 (s, 1H), 9.10-8.95 (m, 2H), 8.83 (d, J=2.1 Hz, 1H), 8.35 (s, 1H), 8.25 (s, 1H), 8.10 (d, J=4.6 Hz, 1H), 8.00-7.83 (m, 2H), 7.37 (d, J=8.3 Hz, 1H), 6.92 (d, J=5.0 Hz, 1H), 6.63 (s, 1H), 2.88 (d, J=4.9 Hz, 3H), 2.30 (s, 3H). MS (ESI) m/z 438.2[M+H]⁺

### Example 2. Synthesis of N-(4-methyl-3-(7-(methylamino)-1,6-naphthyridin-3-yl)phenyl)-2-(trifluoromethyl)isonicotinamide (Compound 2)

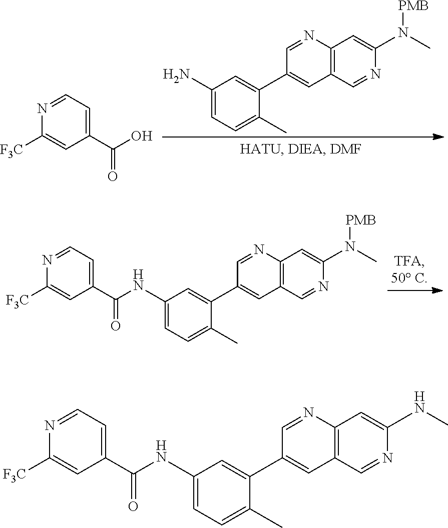

Step 1. A solution of 2-(trifluoromethyl)isonicotinic acid (20 mg, 0.10 mmol), N,N-diisopropylethylamine (38.7 mg, 0.30 mmol), 1-[bis(dimethylamino)methylene]-1H-1,2,3-triazolo[4,5-b]pyridinium 3-oxid hexafluorophosphate (76 mg, 0.20 mmol) and 3-(5-amino-2-methylphenyl)-N-(4-methoxybenzyl)-N-methyl-1,6-naphthyridin-7-amine (38.4 mg, 0.1 mmol) in N,N-dimethylformamide (1 mL) was stirred at room temperature overnight. The mixture was poured into water (5 mL) and extracted with dichloromethane (10 mL×5). The combined organic phases were concentrated and purified by flash chromatography (silica, dichloromethane/methanol=10/1) to afford N-(3-(7-((4-methoxybenzyl)(methyl)amino)-1,6-naphthyridin-3-yl)-4-methylphenyl)-2-(trifluoromethyl)isonicotinamide (40 mg, 0.07 mmol, 71%) as a brown solid. MS (ESI) m/z 558.3 [M+H]⁺

Step 2. A solution of N-(3-(7-((4-methoxybenzyl)(methyl)amino)-1,6-naphthyridin-3-yl)-4-methylphenyl)-2-(trifluoromethyl)isonicotinamide (40 mg, 0.07 mmol) in trifluoroacetic acid (2 mL) was stirred at 50° C. overnight. The mixture was adjusted to pH=8.0 with saturated aqueous sodium bicarbonate solution. The mixture was concentrated and dissolved in dimethyl sulfoxide (1 mL). The solution was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-methyl-3-(7-(methyl amino)-1,6-naphthyridin-3-yl)phenyl)-2-(trifluoromethyl)isonicotinamide (24.5 mg, 0.05 mmol, 80%) as a red solid. ¹H NMR (400 MHz, CD₃OD) δ 9.01 (s, 1H), 8.90 (d, J=4.8 Hz, 1H), 8.83 (d, J=2.4 Hz, 1H), 8.43 (d, J=1.6 Hz, 1H), 8.30 (s, 1H), 8.13-8.12 (m, 1H), 7.78 (d, J=2.4 Hz, 1H), 7.70-7.67 (m, 1H), 7.39 (d, J=8.0 Hz, 1H), 6.68 (s, 1H), 3.00 (s, 3H), 2.34 (s, 3H). MS (ESI) m/z 438.3 [M+H]⁺

### Example 3. Synthesis of N-(4-methyl-3-(7-(methylamino)-1,6-naphthyridin-3-yl)phenyl)-5-(trifluoromethyl)nicotinamide (Compound 3)

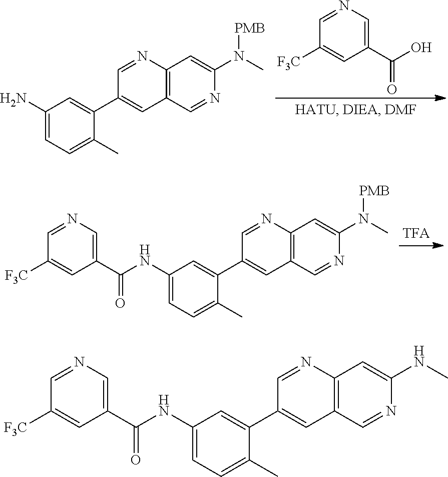

Step 1. A solution of 3-(5-amino-2-methylphenyl)-N-(4-methoxybenzyl)-N-methyl-1,6-naphthyridin-7-amine (57.6 mg, 0.15 mmol), 5-(trifluoromethyl)nicotinic acid (34 mg, 0.18 mmol), 1-[bis(dimethylamino)methylene]-1H-1,2,3-triazolo[4,5-b]pyridinium 3-oxid hexafluorophosphate (70 mg, 0.18 mmol) and N,N-diisopropylethylamine (60 mg, 0.45 mmol) in N,N-dimethylformamide (2 mL) was stirred at 25° C. for 2 hours. The reaction was quenched with water (10 mL) and extracted with ethyl acetate (10 mL×3). The combined organic layers were washed with brine, dried over sodium sulfate, filtered, and concentrated. The residue was purified by flash chromatography (silica, petroleum ether/ethyl acetate=1/1) to afford N-(3-(7-((4-methoxybenzyl)(methyl)amino)-1,6-naphthyridin-3-yl)-4-methylphenyl)-5-(trifluoromethyl)nicotinamide (55 mg, 0.10 mmol, 67%) as a yellow solid. MS (ESI) m/z 558.2 [M+H]⁺

Step 2. A solution of N-(3-(7-((4-methoxybenzyl)(methyl)amino)-1,6-naphthyridin-3-yl)-4-methylphenyl)-5-(trifluoromethyl)nicotinamide (55 mg, 0.10 mmol) in trifluoroacetic acid (1 mL) was stirred at 25° C. for 12 hours. The reaction was quenched with water (3 mL) and extracted with ethyl acetate (10 mL×3). The combined organic layers were washed with brine, dried over sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30/%-70% in 15 min) to afford N-(4-methyl-3-(7-(methylamino)-1,6-naphthyridin-3-yl)phenyl)-5-(trifluoromethyl)nicotinamide (20.0 mg, 0.0456 mmol, 46%) as a yellow solid. ¹H NMR (400 MHz, DMSO-d₆) δ 10.66 (s, 1H), 9.38 (s, 1H), 9.19 (s, 1H), 8.91 (s, 1H), 8.81 (s, 1H), 8.70 (s, 1H), 8.24 (s, 1H), 7.75-7.78 (d, 2H), 7.37-7.39 (s, 1H), 6.91 (s, 1H), 6.62 (s, 1H), 2.86-2.87 (s, 3H), 2.29-2.32 (s, 3H). MS (EST) m/z 438.1 [M+H]⁺

### Example 4. Synthesis of 3-(Dimethylamino)-N-(4-methyl-3-(7-(methylamino)-1,6-naphthyridin-3-yl)phenyl)benzamide (Compound 4)

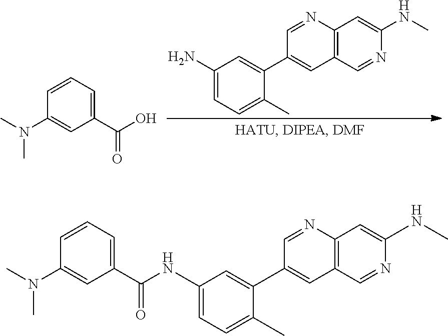

To a solution of 3-(dimethylamino)benzoic acid (50 mg, 0.30 mmol) in N,N-dimethylformamide (2 mL) were added 3-(dimethylamino)-N-(4-methyl-3-(7-(methylamino)-1,6-naphthyridin-3-yl)phenyl)benzamide (80 mg, 0.30 mmol), 1-[bis(dimethylamino)methylene]-1H-1,2,3-triazolo[4,5-b]pyridinium 3-oxid hexafluorophosphate (230 mg, 0.60 mmol) and N,N-diisopropylethylamine (117 mg, 0.90 mmol) at room temperature. The reaction mixture was stirred room temperature for 1 hour. The mixture was diluted with water (20 mL), and then extracted with ethyl acetate (20 mL×2). The combined organic layer was washed with water (20 mL×3) and brine (20 mL), dried over anhydrous sodium sulfate, filtered, and concentrated. The crude residue was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford 3-(dimethylamino)-N-(4-methyl-3-(7-(methylamino)-1,6-naphthyridin-3-yl)phenyl)benzamide (33.0 mg, 0.08 mmol, 27%) as a yellow solid. ¹H NMR (400 MHz, DMSO-d₆) δ 10.17 (s, 1H), 8.98 (s, 1H), 8.81 (d, J=2.0 Hz, 1H), 8.23 (d, J=1.6 Hz, 1H), 7.78-7.70 (m, 2H), 7.34-7.22 (m, 4H), 6.97-6.90 (m, 2H), 6.62 (s, 1H), 2.96 (s, 6H), 2.86 (d, J=4.8 Hz, 3H), 2.21 (s, 3H). MS (ESI) m/z 412.2 [M+H]⁺

### Example 5. Synthesis of 5-Methyl-N-(4-methyl-3-(7-(methylamino)-1,6-naphthyridin-3-yl)phenyl)-4-(trifluoromethyl)picolinamide (Compound 5)

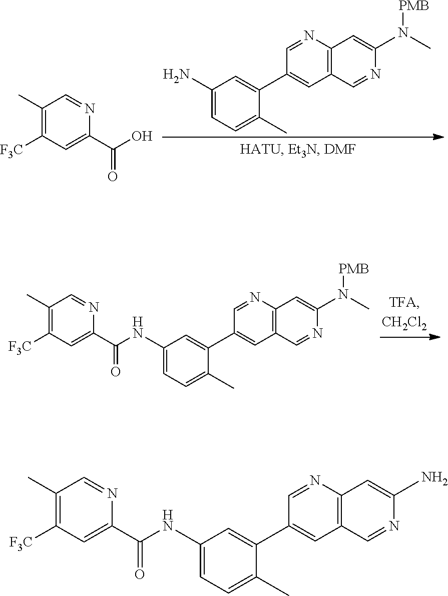

Step 1. A mixture of 5-methyl-4-(trifluoromethyl)picolinic acid (30 mg, 0.15 mmol), 3-(5-amino-2-methylphenyl)-N-(4-methoxybenzyl)-N-methyl-1,6-naphthyridin-7-amine (56 mg, 0.15 mmol), 1-[bis(dimethylamino)methylene]-1H-1,2,3-triazolo[4,5-b]pyridinium 3-oxid hexafluorophosphate (67 mg, 0.18 mmol) and triethylamine (44 mg, 0.44 mmol) in N,N-dimethylformamide (2 mL) was stirred at room temperature for 2 hours. The reaction mixture was treated with water (20 mL). The solid was collected by filtration, washed with water (3 mL) and dried to afford N-(3-(7-((4-methoxybenzyl)(methyl)amino)-1,6-naphthyridin-3-yl)-4-methylphenyl)-5-methyl-4-(trifluoromethyl)picolinamide (40 mg, 0.07 mmol, 47%) as a yellow solid. MS (ESI) m/z 572.1 [M+H]⁺

Step 2. To a solution of N-(3-(7-((4-methoxybenzyl)(methyl)amino)-1,6-naphthyridin-3-yl)-4-methylphenyl)-5-methyl-4-(trifluoromethyl)picolinamide (40 mg, 0.07 mmol) in dichloromethane (0.5 mL) was added trifluoroacetic acid (1 mL). The mixture was stirred at room temperature for 2 hours. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 5-methyl-N-(4-methyl-3-(7-(methylamino)-1,6-naphthyridin-3-yl)phenyl)-4-(trifluoromethyl) picolinamide (22.7 mg, 0.05 mmol, 72%) as a yellow solid. ¹H NMR (400 MHz, DMSO-d₆) δ 10.80 (s, 1H), 8.98 (s, 1H), 8.89 (s, 1H), 8.83 (d, J=2.3 Hz, 1H), 8.3-8.19 (m, 2H), 7.99-7.83 (m, 2H), 7.36 (d, J=8.3 Hz, 1H), 6.90 (q, J=4.9 Hz, 1H), 6.63 (s, 1H), 2.87 (d, J=5.0 Hz, 3H), 2.57 (s, 3H), 2.30 (s, 3H). MS (ESI) m/z 452.1 [M+H]⁺

### Example 6. Synthesis of 3-Chloro-N-(4-methyl-3-(7-(methylamino)-1,6-naphthyridin-3-yl)phenyl)-4-(trifluoromethyl)picolinamide (Compound 6)

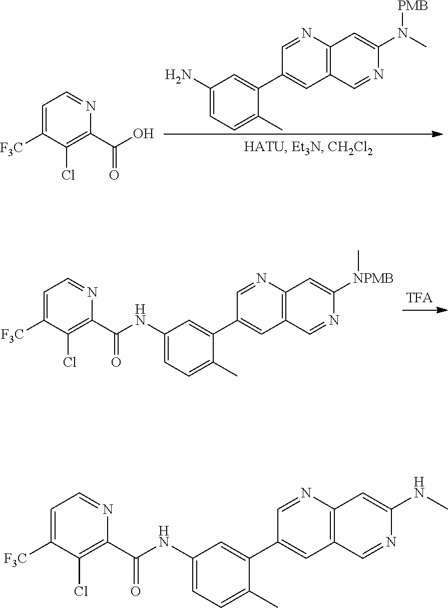

Step 1. A solution of 3-chloro-4-(trifluoromethyl)picolinic acid (0.10 g, 0.44 mmol), 3-(5-amino-2-methyl-phenyl)-N-(4-methoxybenzyl)-N-methyl-1,6-naphthyridin-7-amine (0.17 g, 0.44 mmol), 1-[bis(dimethylamino)methylene]-1H-1,2,3-triazolo[4,5-b]pyridinium 3-oxid hexafluorophosphate (0.25 g, 0.67 mmol) and triethylamine (0.16 g, 1.55 mmol) in dichloromethane (25 mL) was stirred at room temperature for 6 hours. The reaction mixture was diluted with water (40 mL) and extracted with dichloromethane (100 mL). The organic layer was washed with water (20 mL), dried over anhydrous sodium sulfate, filtered, and concentrated to afford 3-chloro-N-(3-(7-((4-methoxybenzyl)(methyl)amino)-1,6-naphthyridin-3-yl)-4-methylphenyl)-4-(trifluoromethyl)picolinamide (110 mg, 0.19 mmol, 42%) as a white solid. MS (ESI) m/z 592.3 [M+H]⁺

Step 2. A solution of 3-chloro-N-(3-(7-((4-methoxybenzyl)methyl)amino)-1,6-naphthyridin-3-yl)-4-methyl-phen-yl)-4-(trifluoromethyl)picolinamide (100 mg, 0.17 mmol) in trifluoroacetic acid (10 mL) was stirred at room temperature for 16 hours. The solvent was removed and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70/% in 15 min) to afford 3-chloro-N-(4-methyl-3-(7-(methylamino)-1,6-naphthyridin-3-yl)phenyl)-4-(trifluoromethyl)picolinamide (59.9 mg, 0.13 mmol, 75%) as a yellow solid. ¹H NMR (400 MHz, DMSO-d₆): δ 10.87 (s, 1H), 8.98 (s, 1H), 8.89 (s, 1H), 8.81 (s, 1H), 8.25 (s, 1H), 8.06 (s, 1H), 7.71 (s, 1H), 7.66 (d, J=8.4 Hz, 1H), 7.36 (d, J=8.4 Hz, 1H), 6.90 (d, J=4.8 Hz, 1H), 6.62 (s, 1H), 2.87 (d, J=4.8 Hz, 3H), 2.28 (s, 3H). MS (ESI) m/z 472.0 [M+H]⁺

### Example 7. Synthesis of N-(4-Methyl-3-(7-(methylamino)-1,6-naphthyridin-3-yl)phenyl)-2-(4-(trifluoromethyl)tetrahydro-2H-pyran-2-yl)acetamide (Compound 7)

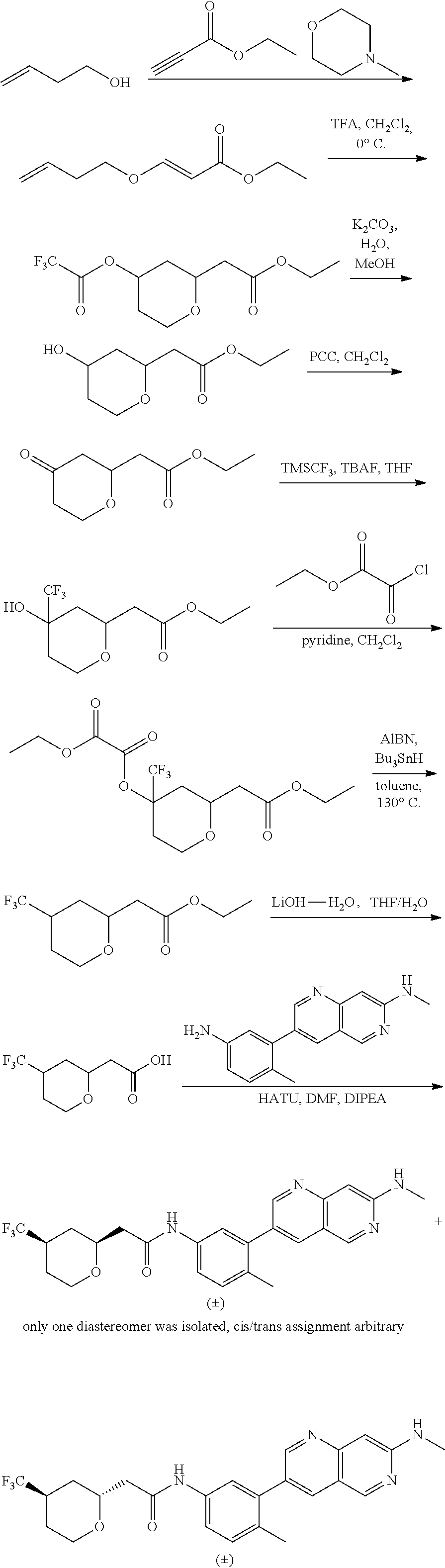

Step 1. To a solution of ethyl propiolate (6.2 g, 63.20 mmol) in ethyl ether (80 mL) were added 4-methylmorpholine (6.3 g. 62.29 mmol) and but-3-en-1-ol (4.25 g, 58.94 mmol) successively. The resulting solution was stirred overnight at 25° C. under nitrogen. The resulting mixture was poured into 0.5 M aqueous acetic acid (150 mL) and extracted with ethyl acetate (150 mL×2). The combined organic layers were dried over sodium sulfate and concenrated under vacuum. The residue was purified by flash chromatography (silica, petroleum ether/ethyl acetate=30/1, then 10/1) to afford ethyl 3-(but-3-enyloxy)acrylate (9.6 g, 56.4 mmol, 96%) as a yellow oil. ¹H NMR (400 MHz, CDCl₃) δ=7.59 (d, J=12.4 Hz, 1H), 5.82 (m, 1H), 5.22-5.09 (m, 3H), 4.16 (q, J=5.3 Hz, 2H), 3.89 (t, J=4.4 Hz, 2H), 2.49-2.44 (m, 2H), 1.27 (t, J=4.7 Hz, 3H). MS (ESI) m/z 171.1 [M−56+H]⁺

Step 2. Trifluoroacetic acid (25 mL) was added to a solution of ethyl 3-(but-3-enyloxy)acrylate (9.6 g, 56.4 mmol) in dichloromethane (150 mL) at 0° C. The reaction mixture was stirred at 0° C. for 2 hours. After completion, the reaction mixture was concentrated in vacuo and diluted with ethyl acetate (200 mL), washed with a cold 1 M sodium bicarbonate solution (100 mL). The organic layer was washed with brine (100 mL), dried over anhydrous sodium sulfate and concentrated under vacuum to afford 2-(2-ethoxy-2-oxoethyl)tetrahydro-2H-pyran-4-yl 2,2,2-trifluoroacetate (14.1 g, 49.6 mmol, 88%) as a yellow oil which was used without further purification. MS (ESI) m/z 285.1 [M+H]⁺

Step 3. Potassium carbonate (13.71 g, 99.2 mmol) in water (200 mL) was added to a solution of 2-(2-ethoxy-2-oxoethyl)tetrahydro-2H-pyran-4-yl 2,2,2-trifluoroacetate (14.1 g, 49.6 mmol) in methanol (80 mL) at 0° C. The reaction mixture was stirred at room temperature for 2 hours. Acetic acid was added to the reaction mixture until pH=7. The resulting solution was extracted with dichloromethane (100 mL×3). The combined organic layers were washed with brine (100 mL), dried over anhydrous sodium sulfate, and concentrated under vacuum to afford ethyl 2-(4-hydroxytetrahydro-2H-pyran-2-yl)acetate (6.8 g, 36.1 mmol, 73%) as a yellow oil which was used without further purification. MS (ESI) m/z 189.1 [M+H]⁺

Step 4. Pyridinium chlorochromate (11.68 g, 54.2 mmol) was added to a solution of ethyl 2-(4-hydroxytetrahydro-2H-pyran-2-yl)acetate (6.8 g, 36.1 mmol) in dichloromethane (200 mL) at room temperature. The reaction mixture was stirred at room temperature for 12 hours. The solution was filtered through silica gel, washing with ethyl acetate (200 mL). The filtrate was concentrated. The residue was purified by flash chromatography (silica, petroleum ether/ethyl acetate=3/1) to afford ethyl 2-(4-oxotetrahydro-2H-pyran-2-yl)acetate (5 g, 26.9 mmol, 74%) as a colorless oil. ¹H NMR (400 MHz, CDCl₃) δ=4.32-4.06 (m, 4H), 3.71 (m, 1H), 2.71-2.33 (m, 6H), 1.28 (t, J=7.0 Hz, 3H). MS (ESI) m/z 187.1 [M+H]⁺

Step 5. Tetrabutylammonium fluoride (1 M in tetrahydrofuran) (2 mL, 2 mmol) was added to a solution of ethyl 2-(4-oxotetrahydro-2H-pyran-2-yl)acetate (372 mg, 2 mmol) and trimethyl(trifluoromethyl)silane (426 mg, 3 mmol) in tetrahydrofuran (10 mL) at room temperature. The reaction mixture was stirred at room temperature for 12 hours. The reaction solution was concentrated. The residue was purified by flash chromatography (silica, petroleum ether/ethyl acetate=2/1) to afford ethyl 2-(4-hydroxy-4-(trifluoromethyl)tetrahydro-2H-pyran-2-yl)acetate (400 mg, 1.56 mmol, 78%) as a colorless oil. ¹H NMR (400 MHz, CDCl₃) δ=4.20-4.11 (m, 3H), 3.99-3.68 (m, 2H), 2.47 (s, 1H), 2.74-2.57 (m, 2H), 2.21-2.05 (m, 2H), 1.76-1.58 (m, 2H), 1.29-1.24 (m, 3H). MS (ESI) m/z 257.0 [M+H]r

Step 6. Ethyl 2-chloro-2-oxoacetate (531 mg, 4.0 mmol) was added to a solution of ethyl 2-(4-hydroxy-4-(trifluoromethyl)tetrahydro-2/H-pyran-2-yl)acetate (500 mg, 2.0 mmol) and pyridine (463 mg, 5.9 mmol) in dichloromethane (15 mL) at 0° C. The reaction mixture was stirred at room temperature for 12 hours. The reaction solution was diluted with dichloromethane (40 mL), washed with 1 N hydrochloric acid (10 mL) and brine (20 mL), dried over sodium sulfate, filtered, and concentrated. The residue was purified by flash chromatography (silica, petroleum ether/ethyl acetate=1/1) to afford 2-(2-ethoxy-2-oxoethyl)-4-(trifluoromethyl)tetrahydro-2H-pyran-4-yl ethyl oxalate (400 mg, 1.11 mmol, 570) as a yellow oil. MS (ESI) m/z 357.1 [M+H]⁺

Step 7. A solution of azobisisobutyronitrile (55 mg, 0.34 mmol) and tributyltin hydride (653 mg, 2.25 mmol) in toluene (4 mL) was added to a solution of 2-(2-ethoxy-2-oxoethyl)-4-(trifluoromethyl)tetrahydro-2H-pyran-4-yl ethyl oxalate (40) mg, 1.11 mmol) in toluene (8 mL) at 130° C. The reaction mixture was stirred at 130° C. for 12 hours. After cooling to room temperature, the reaction solution was diluted with ethyl acetate (40 mL), quenched with saturated aqueous potassium fluoride solution (40 mL), and filtered. The filtrate was separated. The aqueous phase was extracted with ethyl acetate (30 mL×2). The combined organic layers were washed with saturated aqueous potassium fluoride solution (20 mL) and brine (20 mL), dried over sodium sulfate, filtered, and concentrated. The residue was purified by flash chromatography (silica, petroleum ether/ethyl acetate=2/1) to afford ethyl 2-(4-(trifluoromethyl)tetrahydro-2H-pyran-2-yl)acetate (200 mg, 0.83 mmol, 74%) as a yellow oil. MS (EST) m/z 241.3 [M+H]⁺

Step 8. Lithium hydroxide monohydrate (175 mg, 4.2 mmol) was added to a solution of ethyl 2-(4-(trifluoromethyl)tetrahydro-2H-pyran-2-yl)acetate (200 mg, 0.83 mmol) in tetrahydrofuran (6 mL) and water (6 mL) at 0° C. The reaction mixture was stirred at 25° C. for 3 hours. The reaction solution was diluted with water (20 mL) and concentrated to remove tetrahydrofuran. The aqueous layer was acidified to pH=3 with 4 N hydrochloric acid and extracted with ethyl acetate (30 mL×2). The combined organic layers were washed with water (20 mL) and brine (20 mL), dried over sodium sulfate, filtered, and concentrated to afford 2-(4-(trifluoromethyl)tetrahydro-2H-pyran-2-yl)acetic acid (100 mg, 0.47 mmol, 59%) as a yellow oil. The crude was used without further purification. MS (ESI) m/z 213.1 [M+H]⁺

Step 9. To a solution of 3-(5-amino-2-methylphenyl)-N-methyl-1,6-naphthyridin-7-amine (120 mg, 0.45 mmol) in N,N-dimethylformamide (4 mL) was added N,N-diisopropylethylamine (176 mg, 1.36 mmol), 1-[bis(dimethylamino)methylene]-1H-1,2,3-triazolo[4,5-b]pyridinium 3-oxid hexafluorophosphate (259 mg, 0.68 mmol) and 2-(4-(trifluoromethyl)tetrahydro-2H-pyran-2-yl)acetic acid (96 mg, 0.45 mmol). The mixture was allowed to stir at room temperature for 1 hour. The mixture was purified by prep-HPLC (Column; Welch Xtimate 21.2×250 mm C18, 10 μm, Mobile Phase: A: water (10 mmol/L ammonium bicarbonate) B: acetonitrile; B %: 30%-70/o in 15 min) to afford N-(4-methyl-3-(7-(methylamino)-1,6-naphthyridin-3-yl)phenyl)-2-(4-(trifluoromethyl)tetrahydro-2H-pyran-2-yl)acetamide (42.9 mg, 0.09 mmol, 21%) as a yellow solid. Only one diastereomer was detected and isolated, which was arbitrarily assigned as the cis isomer. ¹H NMR (400 MHz, DMSO-d₆) δ 10.00 (s, 1H), 8.97 (s, 1H), 8.76 (d, J=2.4 Hz, 1H), 8.19 (d, J=2.0 Hz, 1H), 7.62 (d, J=2.0 Hz, 1H), 7.51 (d, J₁=2.0 Hz, J₂=8.4 Hz, 1H), 7.27 (d, J=8.4 Hz, 1H), 6.89 (q, J=4.8 Hz, 1H), 6.61 (s, 1H), 3.92-3.96 (m, 1H), 3.77-3.82 (m, 1H), 3.37-3.43 (m, 1H), 2.86 (d, J=5.2 Hz, 3H), 2.69 (s, 1H), 2.52-2.55 (m, 2H), 2.23 (s, 3H), 1.86 (d, J=12.8 Hz, 1H), 1.69 (d, J=11.6 Hz, 1H), 1.35-1.46 (m, 1H), 1.18-1.27 (m, 1H). MS (ESI) m/z 459.3[M+H]⁺

### Example 8. Synthesis of 2-(Difluoromethyl)-N-(4-methyl-3-(7-(methylamino)-1,6-naphthyridin-3-yl)phenyl)isonicotinamide (Compound 8)

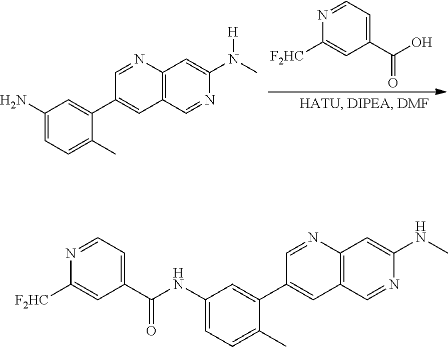 [SMILES: CNc1cc2ncc(-c3cc(N)ccc3C)cc2cn1 ; O=C(O)c1ccnc(C(F)F)c1 ; CNc1cc2ncc(-c3cc(NC(=O)c4ccnc(C(F)F)c4)ccc3C)cc2cn1]

To a solution of 3-(5-amino-2-methylphenyl)-N-methyl-1,6-naphthyridin-7-amine (200 mg, 0.76 mmol) in N,N-dimethylformamide (5 mL) was added N,N-diisopropylethylamine (293 mg, 2.27 mmol), 1-[bis(dimethylamino)methylene]-1H-1,2,3-triazolo[4,5-b]pyridinium 3-oxid hexafluorophosphate (432 mg, 1.14 mmol) and 2-(difluoromethyl)isonicotinic acid (131 mg, 0.76 mmol). The mixture was allowed to stir at room temperature for 1 hour. The mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(difluoromethyl)-N-(4-methyl-3-(7-(methylamino)-1,6-naphthyridin-3-yl)phenyl)isonicotinamide (93.4 mg, 0.22 mmol, 30%) as a yellow solid. ¹H NMR (400 MHz, DMSO-d₆) 10.68 (s, 1H), 8.98 (s, 1H), 8.90 (d, J=5.2 Hz, 1H), 8.81 (d, J=2.4 Hz, 1H), 8.23 (d, J=2.0 Hz, 1H), 8.19 (s, 1H), 8.07 (d, J=4.8 Hz, 1H), 7.77 (d, J=6.4 Hz, 2H), 7.38 (d, J=8.8 Hz, 1H), 6.89-7.22 (m, 2H), 6.62 (s, 1H), 2.87 (d, J=5.2 Hz, 3H), 2.29 (s, 3H). MS (ESI) m/z 420.1 [M+H]⁺

### Example 9. Synthesis of 2-(2-Fluoropropan-2-yl)-N-(4-methyl-3-(7-(methylamino)-1,6-naphthyridin-3-yl)phenyl)isonicotinamide (Compound 9)

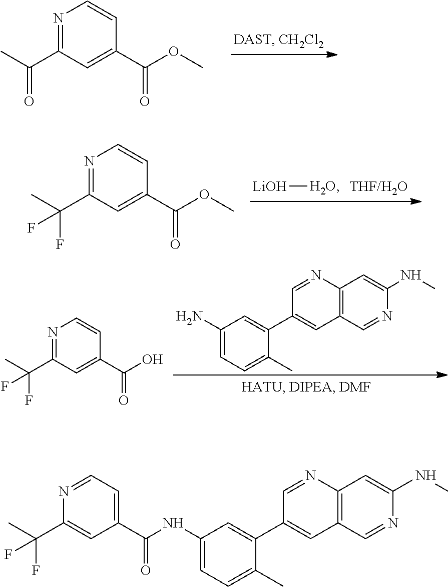

Step 1. A mixture of methyl 2-acetylisonicotinate (300 mg, 1.67 mmol) and diethylaminosulfur trifluoride (675 mg, 4.19 mmol) in dichloromethane (10 mL) was stirred at room temperature for 16 hours. The reaction was quenched with water and extracted with ethyl acetate (10 mL×3). The combined organic phases were washed with brine, dried over sodium sulfate, filtered, concentrated and purified by flash chromatography (silica, ethyl acetate/petroleum ether=1/9) to afford methyl 2-(1,1-difluoroethyl)isonicotinate (100 mg, 0.50 mmol, 29.8%) as a colorless oil. MS (ESI) m/z 202.1 [M+H]⁺

Step 2. A mixture of 2-(1,1-difluoroethyl)isonicotinate (100 mg, 0.50 mmol) and lithium hydroxide monohydrate (42 mg, 1.00 mmol) in tetrahydrofuran (5 mL) and water (0.5 mL) was stirred at room temperature for 2 hours. The reaction was concentrated under reduced pressure, and the residue was re-dissolved in water (5 mL). The resulting solution was acidified with dilute hydrochloric acid to pH=5. The solid was collected by filtration, washed with water and dried to afford 2-(1,1-difluoroethyl)isonicotinic acid (60 mg, 0.32 mmol, 64%) as a white solid. MS (ESI) m/z 188.1[M+H]⁺

Step 3. A mixture of 3-(5-amino-2-methylphenyl)-N-methyl-1,6-naphthyridin-7-amine (42 mg, 0.16 mmol), 2-(1,1-difluoroethyl)isonicotinic acid (30 mg, 0.16 mmol), N,N-diisopropylethylamine (41 mg, 0.32 mmol) and 1-[bis(dimethylamino)methylene]-1H-1,2,3-triazolo[4,5-b]pyridinium 3-oxid hexafluorophosphate (73 mg, 0.19 mmol) in N,N-dimethylformamide (5 mL) was stirred at room temperature for 1 hour. The mixture was quenched with water (10 mL) and extracted with ethyl acetate (5 mL×3). The combined organic phases were washed with brine, dried over sodium sulfate, filtered, concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(1,1-difluoroethyl)-N-(4-methyl-3-(7-(methylamino)-1,6-naphthyridin-3-yl) phenyl)isonicotinamide (20 mg, 0.046 mmol, 29%) as a light yellow solid. ¹H NMR (400 MHz, CD₃OD) δ 8.93 (s, 1H), 8.84-8.76 (m, 2H), 8.81-8.78 (m, 1H), 8.19 (s, 1H), 7.97 (d, J=3.6 Hz, 1H), 7.74-7.68 (m, 2H), 7.38 (d, J=8.2 Hz, 1H), 6.71 (s, 1H), 2.98 (s, 3H), 2.33 (s, 3H), 2.02 (t, J=20.0 Hz, 3H). MS (ESI) m/z 433.8 [M+H]⁺

### Example 10. Synthesis of 2-(2-Fluoropropan-2-yl)-N-(4-methyl-3-(7-(methylamino)-1,6-naphthyridin-3-yl)phenyl)isonicotinamide (Compound 10)

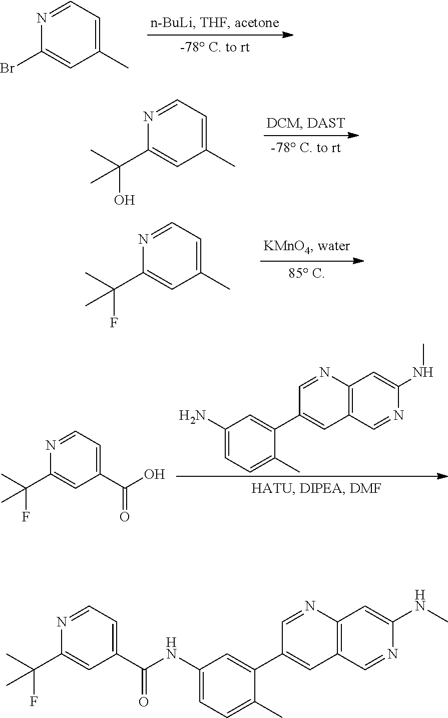

Step 1. To a solution of 2-bromo-4-methylpyridine (1.0 g, 5.85 mmol) in dry tetrahydrofuran (20 mL) under argon protection at −78° C. was added n-butyllithium (2.5M in hexane, 2.80 mL, 7.02 mmol) dropwise. After stirring at this temperature for 30 min, acetone (2 mL) was added. The mixture was allowed to stir at room temperature for another 1 hour. The reaction was quenched with saturated ammonium chloride solution and extracted with ethyl acetate (15 mL×3). The combined organic phases were dried over sodium sulfate, filtered, concentrated, and purified by flash chromatography (silica, ethyl acetate/petroleum ether=1/3) to afford 2-(4-methylpyridin-2-yl)propan-2-ol (500 mg, 3.31 mmol, 57%) as a colorless oil. MS (ESI) m/z 152.3[M+H]⁺

Step 2. To a solution of 2-(4-methylpyridin-2-yl)propan-2-ol (500 mg, 3.31 mmol) in dichloromethane (10 mL) at −78° C. was added diethylaminosulfur trifluoride (799 mg, 4.96 mmol), and the mixture was allowed to stir at room temperature for 3 hours. The reaction was quenched with water and extracted with ethyl acetate (10 mL×3). The combined organic phases were washed with brine, dried over sodium sulfate, filtered, concentrated, and purified by flash chromatography (silica, ethyl acetate/petroleum ether=8/92) to afford 2-(2-fluoropropan-2-yl)-4-methylpyridine (300 mg, 1.96 mmol, 59%) as a colorless oil. MS (EST) m/z 154.3[M+H]⁺

Step 3. A mixture of 2-(2-fluoropropan-2-yl)-4-methylpyridine (250 mg, 1.63 mmol) and potassium permanganate (644 mg, 4.08 mmol) in water (10 mL) stirred at 85° C. for 3 hours. The reaction was cooled to room temperature and acidified with dilute hydrochloric acid to pH=5. The resulting mixture was extracted with ethyl acetate (10 mL×3), and the combined organic layers were washed with brine, dried over anhydrous sodium sulfate, filtered, and concentrated to afford 2-(2-fluoropropan-2-yl)isonicotinic acid (80 mg, 0.44 mmol, 27%) as a white solid. MS (ESI) m/z 184.2[M+H]⁺

Step 4. A mixture of 3-(5-amino-2-methylphenyl)-N-methyl-1,6-naphthyridin-7-amine (116 mg, 0.44 mmol), 2-(2-fluoropropan-2-yl)isonicotinic acid (80 mg, 0.44 mmol), N,N-diisopropylethylamine (114 mg, 0.88 mmol) and 1-[bis(dimethylamino)methylene]-1H-1,2,3-triazolo[4,5-b]pyridinium 3-oxid hexafluorophosphate (200 mg, 0.53 mmol) in N,N-dimethylformamide (5 mL) was stirred at room temperature for 1 hour. The mixture was quenched with water (10 mL) and extracted with ethyl acetate (5 mL×3). The combined organic phases were washed with brine, dried over sodium sulfate, filtered, concentrated and purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(2-fluoropropan-2-yl)-N-(4-methyl-3-(7-(methylamino)-1,6-naphthyridin-3-yl)phenyl)isonicotinamide (26.6 mg, 0.062 mmol, 14%) as a light yellow solid. ¹H NMR (400 MHz, CD₃OD) δ 8.93 (s, 1H), 8.79 (d, J=2.2 Hz, 1H), 8.69 (d, J=5.1 Hz, 1H), 8.26 (d, J=1.6 Hz, 1H), 8.06 (s, 1H), 7.77 (dd, J=5.1, 1.7 Hz, 1H), 7.74-7.67 (m, 2H), 7.37 (d, J=8.1 Hz, 1H), 6.71 (s, 1H), 2.98 (s, 3H), 2.33 (s, 3H), 1.75 (s, 3H), 1.70 (s, 3H). MS (ESI) m/z 429.8 [M+H]⁺

### Example 11. Synthesis of 4-(Difluoromethyl)-N-(4-methyl-3-(7-(methylamino)-1,6-naphthyridin-3-yl)phenyl)picolinamide (Compound 11)

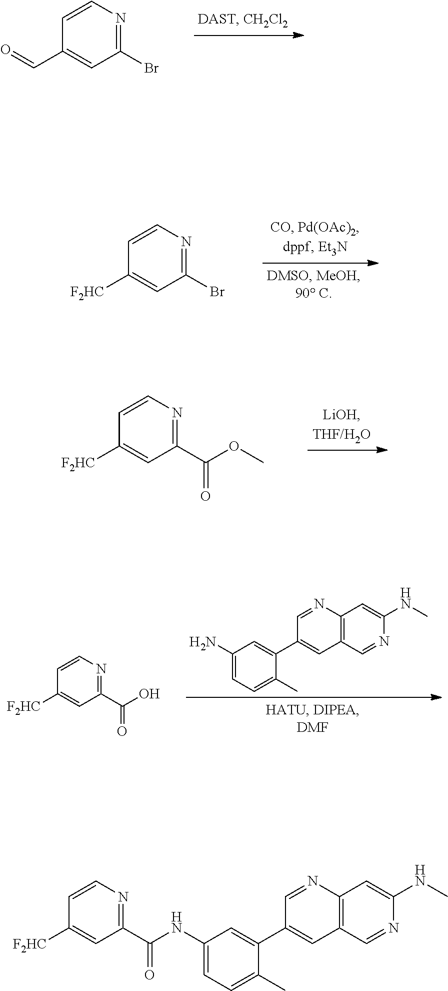 [SMILES: O=Cc1ccnc(Br)c1 ; FC(F)c1ccnc(Br)c1 ; COC(=O)c1cc(C(F)F)ccn1 ; O=C(O)c1cc(C(F)F)ccn1 ; CNc1cc2ncc(-c3cc(N)ccc3C)cc2cn1 ; CNc1cc2ncc(-c3cc(NC(=O)c4cc(C(F)F)ccn4)ccc3C)cc2cn1]

Step 1. To a solution of 2-bromoisonicotinaldehyde (500 mg, 2.68 mmol) in dichloromethane (10 mL) was added diethylaminosulfur trifluoride (863 mg, 5.36 mmol) and the mixture was stirred for 16 hours at room temperature. The mixture was concentrated under reduced pressure. The residue was purified by flash chromatography (silica, ethyl acetate/petroleum ether=1/9) to afford 2-bromo-4-(difluoromethyl)pyridine (390 mg, 1.88 mmol, 70%) as a yellow oil. MS (ESI) m/z 208.1 [M+H]⁺

Step 2. To a solution of 2-bromo-4-(difluoromethyl)pyridine (390 mg, 1.88 mmol) in dimethyl sulfoxide (6 mL) and methanol (4 mL) were added triethylamine (570 mg, 5.64 mmol), 1,1′-bis(diphenylphosphino)ferrocene (416 mg, 0.75 mmol) and palladium acetate (84.4 mg, 0.37 mmol). The resulting mixture was stirred for 16 hours at 90° C. under carbon monoxide atmosphere. After cooling to room temperature, the reaction mixture was quenched with water/ice (100 mL) and extracted with ethyl acetate (3×40 mL). The combined organic layers were washed with brine (2×100 mL) and dried over anhydrous sodium sulfate. After filtration, the filtrate was concentrated under reduced pressure. The residue was purified by flash chromatography (silica, ethyl acetate/petroleum ether=1/1) to afford methyl 4-(difluoromethyl)picolinate (230 mg, 1.22 mmol, 65%) as a yellow solid. MS (ESI) m/z 188.3 [M+H]⁺

Step 3. A solution of methyl 4-(difluoromethyl)picolinate (230 mg, 1.22 mmol) and lithium hydroxide (58 mg, 2.44 mmol) in tetrahydrofuran (4 mL) and water (2 mL) was stirred for 2 hours at room temperature. The mixture was concentrated in vacuo to afford 4-(difluoromethyl)picolinic acid (150 mg, 71%) as a yellow solid, which was used in the next step without further purification. MS (EST) m/z 174.0 [M+H]⁺

Step 4. To a solution of 4-(difluoromethyl)picolinic acid (70 mg, 0.40 mmol) in N,N-dimethylformamide (5 mL) were added 3-(5-amino-2-methylphenyl)-N-methyl-1,6-naphthyridin-7-amine (84 mg, 0.32 mmol), 1-[bis(dimethylamino)methylene]-1H-1,2,3-triazolo[4,5-b]pyridinium 3-oxid hexafluorophosphate (304 mg, 0.80 mmol) and N,N-diisopropylethylamine (155 mg, 1.2 mmol) at room temperature. The reaction mixture was stirred room temperature for 1 hour. The mixture was diluted with water (50 mL), and then extracted with ethyl acetate (3×30 mL). The combined organic layers were washed with brine (2×50 mL), dried over anhydrous sodium sulfate, filtered, and concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(difluoromethyl)-N-(4-methyl-3-(7-(methylamino)-1,6-naphthyridin-3-yl)phenyl)picolinamide (8.0 mg, 0.02 mmol, 5%) as a yellow solid. ¹H NMR (400 MHz, DMSO-d₆) δ 10.73 (s, 1H), 8.98 (s, 1H), 8.92 (d, J=4.8 Hz, 1H), 8.30 (d, J=2.4 Hz, 1H), 8.27-8.24 (m, 2H), 7.94-7.87 (m, 3H), 7.39-7.12 (m, 2H), 6.91-6.90 (m, 1H), 6.62 (s, 1H), 2.86 (d, J=4.8 Hz, 3H), 2.29 (s, 3H). MS (ESI) m/z 420.1 [M+H]⁺

### Example 12. Synthesis of 4-(1,1-Difluoroethyl)-N-(4-methyl-3-(7-(methylamino)-1,6-naphthyridin-3-yl)phenyl)picolinamide (Compound 12)

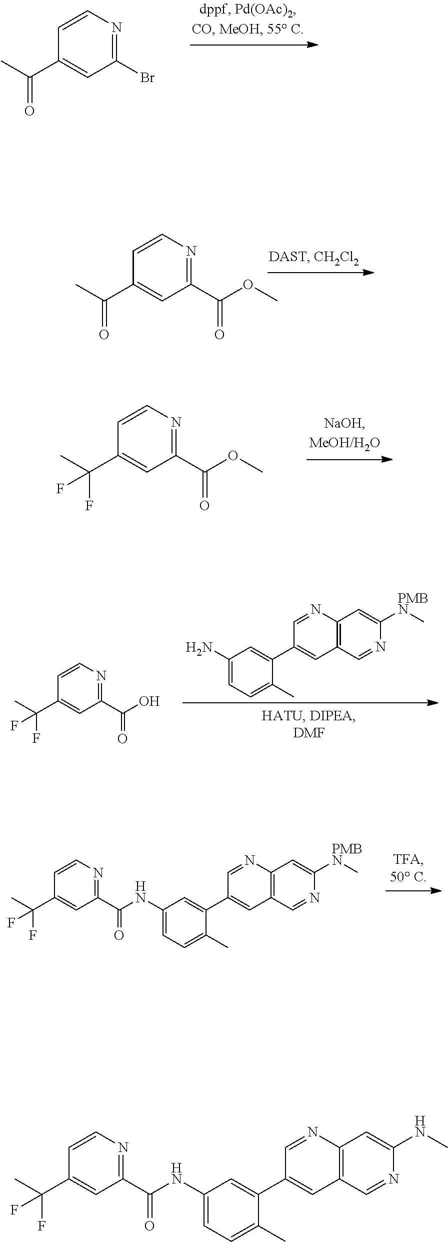

Step 1. To a solution of 1-(2-bromopyridin-4-yl)ethan-1-one (3 g, 15.15 mmol) in ethanol (20 mL) was added 1,1′-bis(diphenylphosphino)ferrocene-palladium(II) dichloride (830 mg, 1.5 mmol) and palladium (II) acetate at room temperature. The reaction mixture was stirred at 55° C. under carbon monoxide for 16 hours. The mixture was concentrated. The residue was purified by flash chromatography (silica, ethyl acetate/petroleum ether=1/4) to afford methyl 4-acetylpicolinate (860 mg, crude) as a colorless oil. MS (ESI) m/z 180.1[M+H]⁺

Step 2. To a solution of methyl 4-acetylpicolinate (250 mg, 1.4 mmol) in dichloromethane (5 mL) was added diethylaminosulfur trifluoride (670 mg, 4.2 mmol) at room temperature. The reaction mixture was stirred at room temperature for 24 hours. The mixture was diluted with water (10 mL) at 0° C., and then extracted with ethyl acetate (30 mL×3). The combined organic layers were washed with brine (30 mL), dried over anhydrous sodium sulfate, and filtered. The filtrate was concentrated. The residue was purified by flash chromatography (silica, ethyl acetate/petroleum ether=1/3) to afford methyl 4-(1,1-difluoroethyl)picolinate (55 mg, crude) as a colorless oil. MS (ESI) m/z 202.1[M+H]⁺

Step 3. To a solution of methyl 4-(1,1-difluoroethyl)picolinate (55 mg, 0.27 mmol) in methanol (5 mL) and water (I mL) was added sodium hydroxide (32.8 mg, 0.82 mmol) at room temperature. The reaction mixture was stirred at room temperature for 2 hours. The mixture was purified by reverse phase HPLC to afford 4-(1,1-difluoroethyl)picolinic acid (32 mg, 0.17 mmol) as a white solid. MS (ESI) m/z 188.1[M+H]⁺

Step 4. A solution of 4-(1,1-difluoroethyl)picolinic acid (15 mg, 0.08 mmol), N,N-diisopropylethylamine (30 mg, 0.24 mmol), 1-[bis(dimethylamino)methylene]-1H-1,2,3-triazolo[4,5-b]pyridinium 3-oxid hexafluorophosphate (60 mg, 0.08 mmol) and 3-(5-amino-2-methylphenyl)-N-(4-methoxybenzyl)-N-methyl-1,6-naphthyridin-7-amine (30.7 mg, 0.08 mmol) in N,N-dimethylformamide (3 mL) was stirred at room temperature for 2 hours. The mixture was diluted with water (10 mL), and then extracted with ethyl acetate (20 mL×3). The combined organic layers were washed with brine (10 mL), dried over anhydrous sodium sulfate, and filtered. The filtrate was concentrated under reduced pressure to afford 4-(1,1-difluoroethyl)-N-(3-(7-((4-methoxybenzyl)(methyl)amino)-1,6-naphthyridin-3-yl)-4-methylphenyl)picolinamide (40 mg, crude) as a yellow solid. MS (ESI) m/z 554.3 [M+H]⁺

Step 5. A mixture of 4-(1,1-difluoroethyl)-N-(3-(7-((4-methoxybenzyl)(methyl)amino)-1,6-naphthyridin-3-yl)-4-methylphenyl)picolinamide (40 mg, crude) in trifluoroacetic acid (5 mL) was stirred at 50° C. for 4 hours. The reaction was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-methyl-3-(7-(methylamino)-1,6-naphthyridin-3-yl)phenyl)-4-(trifluoromethyl)picolinamide (12.7 mg, 0.029 mmol) as a yellow solid. ¹H NMR (400 MHz, DMSO-d₆) δ 10.79 (s, 1H), 8.98 (s, 1H), 8.90 (d, J=5.1 Hz, 1H), 8.83 (d, J=2.3 Hz, 1H), 8.24 (s, 2H), 7.99-7.79 (m, 3H), 7.36 (d, J=8.3 Hz, 1H), 6.97-6.83 (m, 1H), 6.63 (s, 1H), 2.87 (d, J=4.9 Hz, 3H), 2.30 (s, 3H), 2.05 (t, J=19.3 Hz, 3H). MS (ESI) m/z 434.1[M+H]⁺

### Example 13. Synthesis of 4-(2-Fluoropropan-2-yl)-N-(4-methyl-3-(7-(methylamino)-1,6-naphthyridin-3-yl)phenyl)picolinamide (Compound 13)

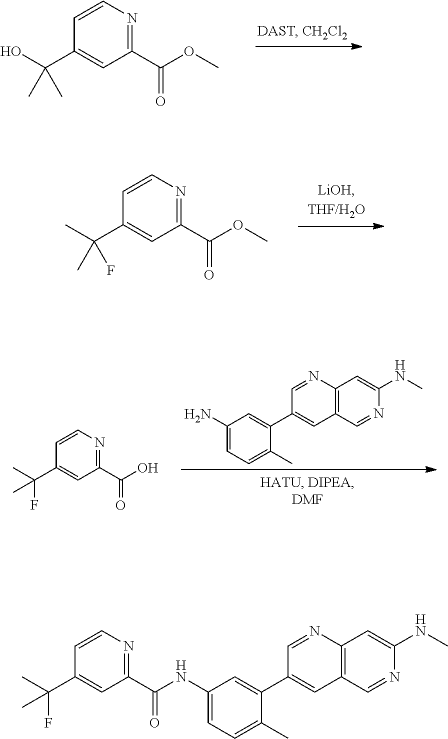 [SMILES: COC(=O)c1cc(C(C)(C)O)ccn1 ; COC(=O)c1cc(C(C)(C)F)ccn1 ; CC(C)(F)c1ccnc(C(=O)O)c1 ; CNc1cc2ncc(-c3cc(N)ccc3C)cc2cn1 ; CNc1cc2ncc(-c3cc(NC(=O)c4cc(C(C)(C)F)ccn4)ccc3C)cc2cn1]

Step 1. To a solution of methyl 4-(2-hydroxypropan-2-yl)picolinate (150 mg, 0.76 mmol) in dichloromethane (5 mL) was added diethylaminosulfur trifluoride (245 mg, 1.52 mmol) and the mixture was stirred for 16 hours at room temperature. The mixture was concentrated under reduced pressure. The residue was purified by flash chromatography (silica, ethyl acetate/petroleum ether=1/9) to afford methyl 4-(2-fluoropropan-2-yl)picolinate (90 mg, 0.45 mmol, 60%) as a yellow oil. MS (EST) m/z 198.3 [M+H]⁺

Step 2. A solution of methyl 4-(2-fluoropropan-2-yl)picolinate (90 mg, 0.45 mmol) and lithium hydroxide (22 mg, 0.90 mmol) in tetrahydrofuran (2 mL) and water (1 mL) was stirred for 2 hours at room temperature. The mixture was concentrated in vacuo to afford 4-(2-fluoropropan-2-yl)picolinic acid (60 mg, 0.33 mmol, 72%) as a yellow solid. MS (ESI) m/z 184.0 [M+H]⁺

Step 3. To a solution of 4-(2-fluoropropan-2-yl)picolinic acid (60 mg, 0.33 mmol) in N,N-dimethylformamide (5 mL) were added 3-(5-amino-2-methylphenyl)-N-methyl-1,6-naphthyridin-7-amine (40 mg, 0.16 mmol), 1-[bis(dimethylamino)methylene]-1H-1,2,3-triazolo[4,5-b]pyridinium 3-oxid hexafluorophosphate (251 mg, 0.66 mmol) and N,N-diisopropylethylamine (128 mg, 0.99 mmol) at room temperature. The reaction mixture was stirred room temperature for 3 hours. The mixture was diluted with water (50 mL), and then extracted with ethyl acetate (3×30 mL). The combined organic layers were washed with brine (2×50 mL), dried over anhydrous sodium sulfate, filtered, and concentrated. The crude residue was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-fluoropropan-2-yl)-N-(4-methyl-3-(7-(methylamino)-1,6-naphthyridin-3-yl)phenyl)picolinamide (2.1 mg, 0.005 mmol, 1.5%) as a yellow solid. ¹H NMR (400 MHz, DMSO-d₆) δ 10.72 (s, 1H), 8.98 (s, 1H), 8.83 (d, J=2.0 Hz, 1H), 8.75 (d, J=4.8 Hz, 1H), 8.24 (s, 1H), 8.15 (s, 1H), 7.93-7.88 (m, 2H), 7.71-7.69 (m, 1H), 7.36-7.34 (m, 1H), 6.91-6.87 (m, 1H), 6.62 (s, 1H), 2.87 (d, J=4.8 Hz, 3H), 2.29 (s, 3H), 1.73 (s, 3H), 1.67 (s, 3H), 1.23 (s, 3H). MS (ESI) m/z 430.2 [M+H]⁺

### Example 14. Synthesis of 4-(2-Cyanopropan-2-yl)-N-(4-methyl-3-(7-(methylamino)-1,6-naphthyridin-3-yl)phenyl)picolinamide (Compound 14)

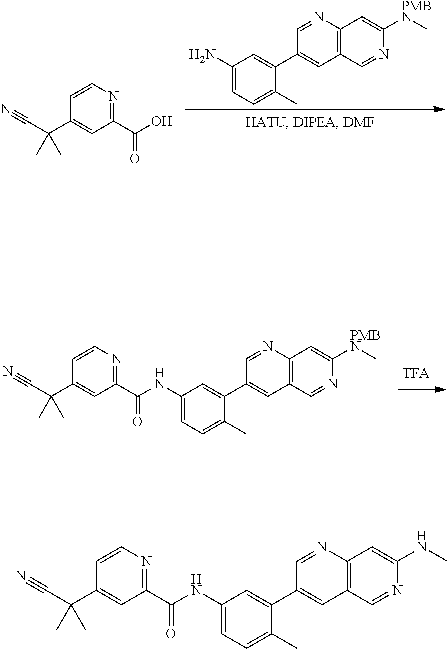

Step 1. To a solution of 4-(2-cyanopropan-2-yl)picolinic acid (30 mg, 0.16 mmol) in N,N-dimethylformamide (2 mL) were added 3-(5-amino-2-methylphenyl)-N-(4-methoxybenzyl)-N-methyl-1,6-naphthyridin-7-amine (60 mg, 0.16 mmol), 1-[bis(dimethylamino)methylene]-1H-1,2,3-triazolo[4,5-b]pyridinium 3-oxid hexafluorophosphate (67 mg, 0.18 mmol) and N,N-diisopropylethylamine (62 mg, 0.48 mmol) at room temperature. The reaction mixture was stirred room temperature for 1 hour. The mixture was diluted with water (20 mL) and extracted with ethyl acetate (20 mL×2). The combined organic layers were washed with water (20 mL×3) and brine (20 mL), dried over anhydrous sodium sulfate, filtered, and concentrated. The crude residue was purified by flash chromatography (silica, petroleum ether/ethyl acetate=1/4) to afford 4-(2-cyanopropan-2-yl)-N-(3-(7-((4-methoxybenzyl)(methyl)amino)-1,6-naphthyridin-3-yl)-4-methylphenyl)picolinamide (45 mg, 0.081 mmol, 51%) as a yellow solid. MS (ESI) m/z 556.8 [M+H]⁺

Step 2. A solution of 4-(2-cyanopropan-2-yl)-N-(3-(7-((4-methoxybenzyl)(methyl)amino)-1,6-naphthyridin-3-yl)-4-methylphenyl)picolinamide (45.0 mg, 0.081 mmol) in trifluoroacetic acid (4 mL) was stirred at room temperature for 2 hours. Then the mixture was purified by prep-HPLC (column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(4-methyl-3-(7-(methylamino)-1,6-naphthyridin-3-yl)phenyl)picolinamide (23.5 mg, 0.054 mmol, 67%) as a yellow solid. ¹H NMR (500 MHz, DMSO-d₆) δ 10.74 (s, 1H), 8.99 (s, 1H), 8.84 (s, 1H), 8.80 (d, J=5.5 Hz, 1H), 8.27 (d, J=2.0 Hz, 1H), 8.25 (s, 1H), 7.94 (d, J=2.0 Hz, 1H), 7.90 (dd, J=8.0 Hz, 2.0 Hz, 1H), 7.84 (q, J=2.0 Hz, 1H), 7.36 (d, J=8.5 Hz, 1H), 6.90 (s, 1H), 6.63 (s, 1H), 2.88 (d, J=5.0 Hz, 3H), 2.30 (s, 3H), 1.76 (s, 6H). MS (EST) m/z 436.8 [M+H]⁺

### Example 15. Synthesis of 4-(1-Cyanocyclobutyl)-N-(4-methyl-3-(7-(methylamino)-1,6-naphthyridin-3-yl)phenyl)picolinamide (Compound 15)

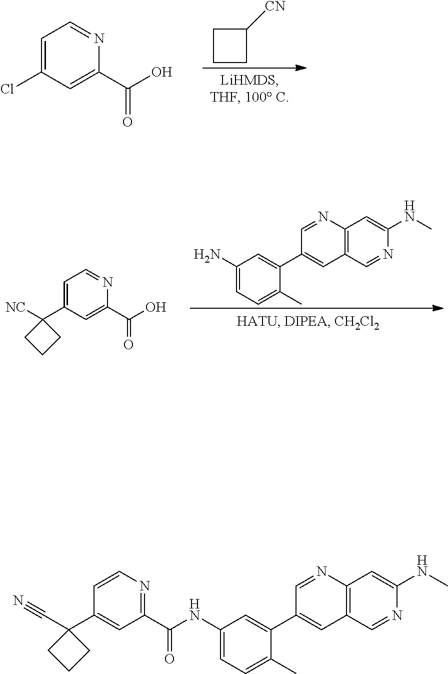 [SMILES: O=C(O)c1cc(Cl)ccn1 ; N#CC1CCC1 ; N#CC1(c2ccnc(C(=O)O)c2)CCC1 ; CNc1cc2ncc(-c3cc(N)ccc3C)cc2cn1 ; CNc1cc2ncc(-c3cc(NC(=O)c4cc(C5(C#N)CCC5)ccn4)ccc3C)cc2cn1]

Step 1. A mixture of 4-chloropicolinic acid (I g, 6.37 mmol), cyclobutanecarbonitrile (1.54 g, 19.11 mmol) and lithium hexamethyldisilazide (124 mL, 19.11 mmol) in tetrahydrofuran (10 mL) was stirred at 100° C. for 30 mm. After cooling to room temperature, the reaction was quenched with saturated ammonium chloride solution (10 mL) and adjusted to pH=3-4 with 6 N hydrochloric acid. The mixture was extracted with 15% isopropanol/dichloromethane (10 mL x 3). The combined organic layers were washed with brine (20 mL), dried over sodium sulfate, filtered, and concentrated to afford 4-(1-cyanocyclobutyl)picolinic acid (260 mg, 1.28 mmol, 20%) as a yellow solid. MS (ESI) m/z 203.2 [M+H]⁺

Step 2. A mixture of 4-(1-cyanocyclobutyl)picolinic acid (83 mg, 0.41 mmol), 3-45-amino-2-methylphenyl)-N-methyl-1,6-naphthyridin-7-amine (90 mg, 0.34 mmol), 1-[bis(dimethylamino)methylene]-1H-1,2,3-triazolo[4,5-b]pyridinium 3-oxid hexafluorophosphate (260 mg, 0.68 mmol) and N,N-diisopropylethylamine (220 mg, 1.7 mmol) in dichloromethane (10 mL) was stirred at 25° C. for 3 hours. The reaction was poured into water (10 mL) and extracted with dichloromethane (10 mL×3). The combined organic layers were washed with brine (20 mL), dried over sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(1-cyanocyclobutyl)-N-(4-methyl-3-(7-(methylamino)-1,6-naphthyridin-3-yl)phenyl)picolinamide (44.9 mg, 0.10 mmol, 29%) as a yellow solid. ¹H NMR (400 MHz, DMSO-d₆) δ 10.77 (s, 11H), 8.99 (s, 1H), 8.82 (dd, J=8.6, 3.6 Hz, 2H), 8.23 (dd, J=16.3, 1.6 Hz, 2H), 7.95 (d, J=2.0 Hz, 11H), 7.90 (dd, J=8.3, 2.1 Hz, 1H), 7.84 (dd, J=5.1, 1.9 Hz, 1H), 7.36 (d, J=8.4 Hz, 1H), 6.91 (d, J=5.0 Hz, 1H), 6.63 (s, 1H), 2.87 (d, J=4.9 Hz, 3H), 2.85-2.77 (m, 2H), 2.76-2.67 (m, 2H), 2.38-2.32 (m, 1H), 2.30 (s, 3H), 2.14-2.04 (m, 1H). MS (ESI) m/z 449.1 [M+H]⁺

### Example 16. Synthesis of 4-(1-Cyanocyclopropyl)-N-(4-methyl-3-(7-(methylamino)-1,6-naphthyridin-3-yl)phenyl)picolinamide (Compound 16)

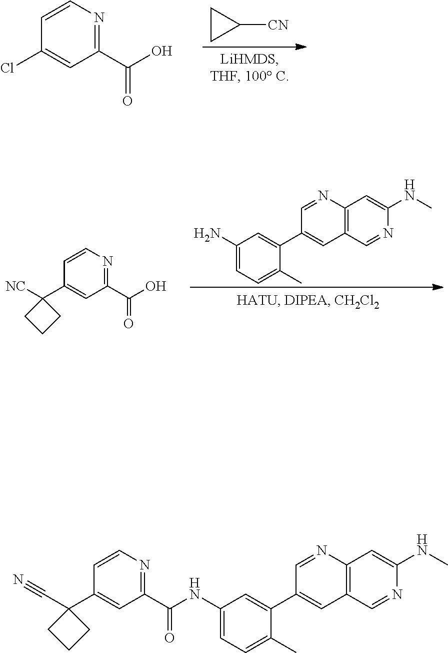 [SMILES: O=C(O)c1cc(Cl)ccn1 ; N#CC1CC1 ; N#CC1(c2ccnc(C(=O)O)c2)CCC1 ; CNc1cc2ncc(-c3cc(N)ccc3C)cc2cn1 ; CNc1cc2ncc(-c3cc(NC(=O)c4cc(C5(C#N)CCC5)ccn4)ccc3C)cc2cn1]

Step 1. A mixture of 4-chloropicolinic acid (I g, 6.37 mmol), cyclopropanecarbonitrile (1.28 g, 19.11 mmol) and lithium hexamethyldisilazide (12.4 mL, 19.11 mmol) in tetrahydrofuran (10 mL) was stirred at 100° C. for 30 min. The reaction mixture was quenched with saturated ammonium chloride solution (10 mL) and adjusted to pH=3-4 with 6 N hydrochloric acid. The mixture was extracted with 15% isopropanol/dichloromethane (10 mL×3). The combined organic layers were washed with brine (20 mL), dried over sodium sulfate, filtered, and concentrated to afford 4-(1-cyanocyclopropyl)picolinic acid (460 mg, 2.45 mmol, 38%) as a yellow solid.

Step 2. A mixture of 4-(1-cyanocyclopropyl)picolinic acid (77 mg, 0.41 mmol), 3-(5-amino-2-methylphenyl)-N-methyl-1,6-naphthyridin-7-amine (90 mg, 0.34 mmol), I-[bis(dimethylamino)methylene]-1H-1,2,3-triazolo[4,5-b]pyridinium 3-oxid hexafluorophosphate (260 mg, 0.68 mmol) and N,N-diisopropylethylamine (220 mg, 1.7 mmol) in dichloromethane (10 mL) was stirred at 25° C. for 3 hours. The reaction was poured into water (10 mL) and extracted with dichloromethane (10 mL×3). The combined organic layers were washed with brine (20 mL), dried over sodium sulfate, filtered, and concentrated. The residue was purified by prep-HPLC (Column; Welch Xtimate 21.2×250 mm C18, 10 μm, Mobile Phase: A: water (10 mmol/L ammonium bicarbonate) B: acetonitrile; B %: 30%-70% in 15 min) to afford 4-(1-cyanocyclopropyl)-N-(4 methyl-3-(7-(methylamino)-1,6-naphthyridin-3-yl)phenyl)picolinamide (51.1 mg, 0.117 mmol, 34%) as a yellow solid. ¹H NMR (400 MHz, DMSO-d₆) δ 10.74 (s, 11H), 8.98 (s, 1H), 8.83 (d, J=1.8 Hz, 1H). 8.70 (d, J=5.1 Hz, 1H), 8.24 (s, 1H), 8.07 (s, 1H), 8.00-7.83 (m, 2H), 7.53 (d, J=3.5 Hz, 1H), 7.36 (d, J=8.1 Hz, 1H), 6.91 (d, J=: 4.7 Hz, 1H), 6.63 (s, 1H), 2.87 (d, J=4.7 Hz, 3H), 2.30 (s, 3H), 2.00 (d, J=2.3 Hz, 2H), 1.78 (d, J=2.5 Hz, 2H). MS (ESI) m/z 435.1 [M+H]⁺

### Example 17. Synthesis of 2-(3,3-Difluorocyclobutyl)-N-(4-methyl-3-(7-(methylamino)-1,6-naphthyridin-3-yl)phenyl)isonicotinamide (Compound 17)

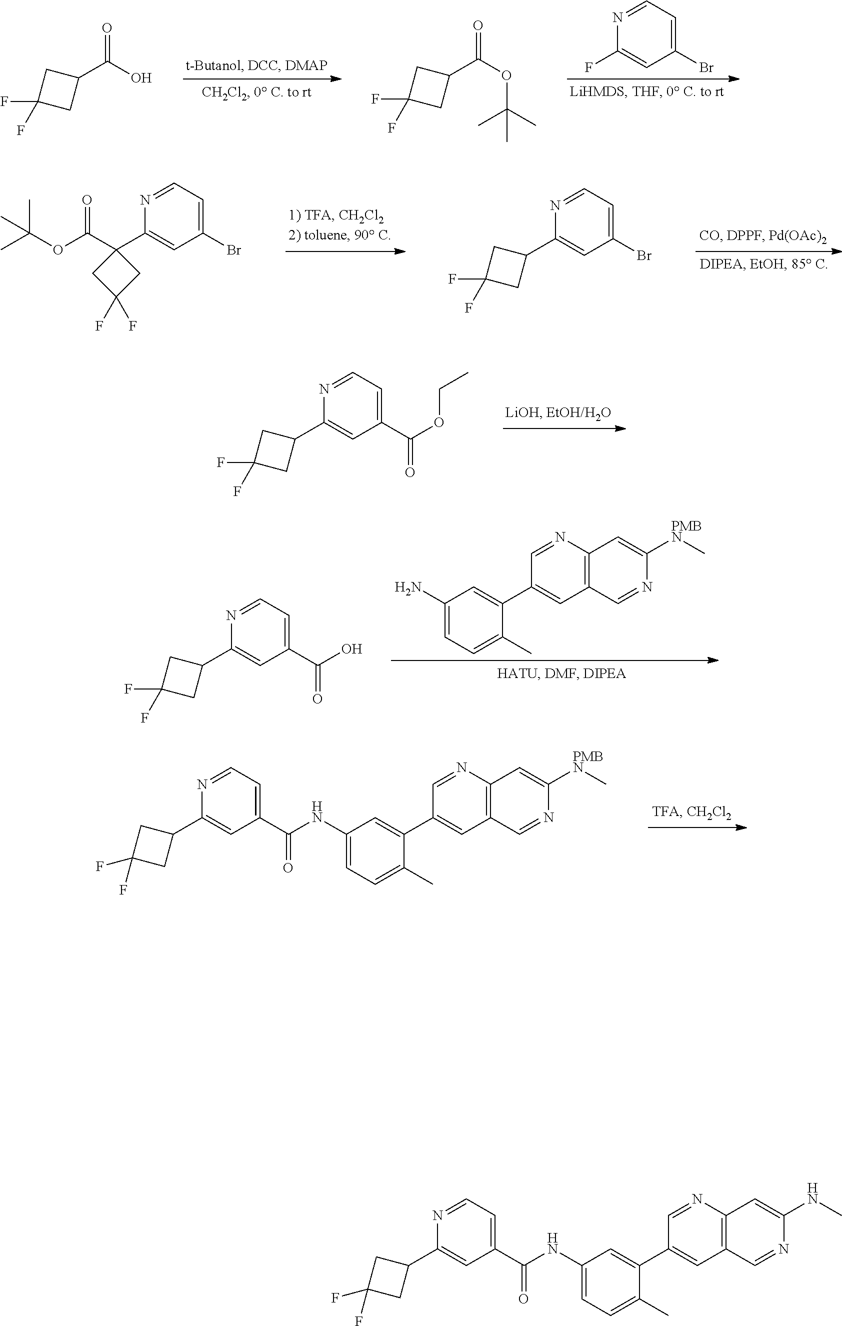

Step 1. 3,3-Difluorocyclobutane-1-carboxylic acid (5.0 g, 36.8 mmol) was dissolved in dichloromethane (20 mL) and cooled in an ice bath. To the solution was added N,N-dimethylpyridin-4-amine (448.5 mg, 3.68 mmol) portion-wise, followed by tert-butanol (5.4 g, 73.5 mmol) in one portion. A 1 M solution of N,N′-dicyclohexylcarbodiimide in dichloromethane (8.3 g, 40.4 mmol) was added dropwise, keeping the temperature below 10° C. The resulting slurry was warmed up to room temperature and stirred for 16 hours. The solid was removed by filtration. The filtrate was washed sequentially with 2 N hydrochloric acid (60 mL×2), water (60 mL×2), and saturated aqueous sodium bicarbonate solution (50 mL×2). The combine organic layers were dried over sodium sulfate, filtered, and concentrated to afford a crude product as a mixture of white solid and yellow oil. To the mixture was added pentane (60 mL) and then filtered through a silica gel pad eluting with pentane. The filtrate was concentrated to afford tert-butyl 3,3-difluorocyclobutane-1-carboxylate (4.0 g. 20.8 mmol, 57%) as a colorless oil. ¹H NMR (400 MHz, CDCl₃) δ 2.84-2.72 (m, 5H), 1.46 (s, 9H).

Step 2. A solution of lithium hexamethyldisilazide (12.1 mL, 19.3 mmol, 1.6 M solution in tetrahydrofuran) was added dropwise to a solution of 4-bromo-2-fluoropyridine (2.6 g, 14.9 mmol) and tert-butyl 3,3-difluorocyclobutane-1-carboxylate (3.4 g, 17.8 mmol) in tetrahydrofuran (30 mL) at 0° C. The reaction mixture was stirred for 20 min at the same temperature, and then allowed to warm to room temperature and stirred for 4 hours. The reaction was quenched with saturated aqueous ammonium chloride solution (50 mL) and extracted with ethyl acetate (100 mL×3). The combined organic layers were washed with brine (150 mL). The organic phase was dried over sodium sulfate, filtered, and concentrated in vacuo to afford a crude product, which was purified by flash chromatography (silica, 0-20% ethyl acetate in petroleum ether) to afford tert-butyl 1-(4-bromopyridin-2-yl)-3,3-difluorocyclobutane-1-carboxylate (1.0 g, 2.88 mmol, 22%) as a yellow oil. MS (ESI) m/z 347.0 [M+H]⁺

Step 3. To a solution of tert-butyl 1-(4-bromopyridin-2-yl)-3,3-difluorocyclobutane-1-carboxylate (1.0 g, 2.88 mmol) in dichloromethane (10 mL) was added trifluoroacetic acid (1.2 g, 11.52 mmol) at room temperature under nitrogen atmosphere. The resulting solution was stirred for 16 hours. The solvent was removed in vacuo and toluene (20 mL) was added. The resulting mixture was warmed up to 90° C. and stirred for 2 hours. The reaction mixture was concentrated in vacuo. The crude product was purified by flash chromatography (silica, ethyl acetate/petroleum ether=1:4) to afford 4-bromo-2-(3,3-difluorocyclobutyl) pyridine (600 mg, 2.43 mmol, 85%) as a colorless oil. MS (ESI) m/z 248.0 [M+H]⁺

Step 4. A solution of 4-bromo-2-(3,3-difluorocyclobutyl)pyridine (600 mg, 2.43 mmol), 1,1′-bis(diphenylphosphino)ferrocene (269 mg, 0.49 mmol), palladium (II) acetate (57 mg, 0.24 mmol) and N,N-diisopropylethylamine (940 mg, 7.29 mmol) in ethanol (15 mL) was stirred at 85° C. for 16 hours under carbon monoxide atmosphere. On completion, the reaction mixture was cooled to room temperature and used without further purification. MS (ESI) m/z 242.2 [M+H]⁺

Step 5. A mixture of ethyl 2-(3,3-difluorocyclobutyl)isonicotinate and lithium hydroxide (175 mg, 7.3 mmol) in water (5 mL) was stirred at room temperature for 1 hour. Then the organic solvent was removed. The residue was washed with ethyl acetate (20 mL). The aqueous phase was acidified with 1 N hydrochloric acid until no more precipitate formed. The mixture was filtered. The solid was washed with water (20 mL) and dried to afford 2-(3,3-difluorocyclobutyl)isonicotinic acid (260 mg, 1.22 mmol, 51% two steps) as a white solid. MS (ESI) m/z 214.1 [M+H]⁺

Step 6. To a solution of 3-(5-amino-2-methylphenyl)-N-(4-methoxybenzyl)-N-methyl-1,6-naphthyridin-7-amine (99 mg, 0.26 mmol) in N,N-dimethylformamide (4 mL) was added N,N-diisopropylethylamine (100 mg, 0.77 mmol), 1-[bis(dimethylamino)methylene]-1H-1,2,3-triazolo[4,5-b]pyridinium 3-oxid hexafluorophosphate (147 mg, 0.39 mmol) and 2-(3,3-difluorocyclobutyl)isonicotinic acid (55 mg, 0.26 mmol). After stirring at room temperature for 1 h, the mixture was extracted with ethyl acetate (30 mL×3). The combined organic layers were washed with brine (30 mL), dried over anhydrous sodium sulfate, and filtered. The filtrate was concentrated under reduced pressure. The residue was purified by flash chromatography (silica, ethyl acetate) to afford 2-(3,3-difluorocyclobutyl)-N-(3-(7-((4-methoxybenzyl)(methyl)amino)-1,6-naphthyridin-3-yl)-4-methylphenyl)isonicotinamide (70 mg, 0.12 mmol, 50%) as a yellow solid. MS (ESI) m/z 580.2 [M+H]⁺

Step 7. A mixture of 2-(3,3-difluorocyclobutyl)-N-(3-(7-((4-methoxybenzyl)(methyl)amino)-1,6-naphthyridin-3-yl)-4-methylphenyl)isonicotinamide (70 mg, 0.12 mmol) in trifluoroacetic acid (4 mL) was stirred at room temperature for 16 hours. The reaction was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70)% in 15 min) to afford 2-(3,3-difluorocyclobutyl)-N-(4-methyl-3-(7-(methylamino)-1,6-naphthyridin-3-yl)phenyl)isonicotinamide (35.2 mg, 0.08 mmol, 64%) as a yellow solid. ¹H NMR (400 MHz, DMSO-d₆) δ 10.53 (s, 1H), 8.99 (s, 1H), 8.77-8.82 (m, 2H), 7.81 (s, 1H), 8.24 (s, 1H), 2.29 (s, 3H), 2.87 (d, J=4.8 Hz, 3H), 7.74 (d, J=7.2 Hz, 3H), 7.36 (d, J=8.8 Hz, 1H), 6.92 (d, J=4.8 Hz, 1H), 6.62 (s, 1H), 3.61-3.66 (m, 1H), 2.92-3.00 (m, 4H). M S (ESI) m/z 460.0 [M+H]⁺

### Example 18. Synthesis of 2-(1-Fluorocyclopropyl)-N-(4-methyl-3-(7-(methylamino)-1,6-naphthyridin-3-yl)phenyl)isonicotinamide (Compound 18)

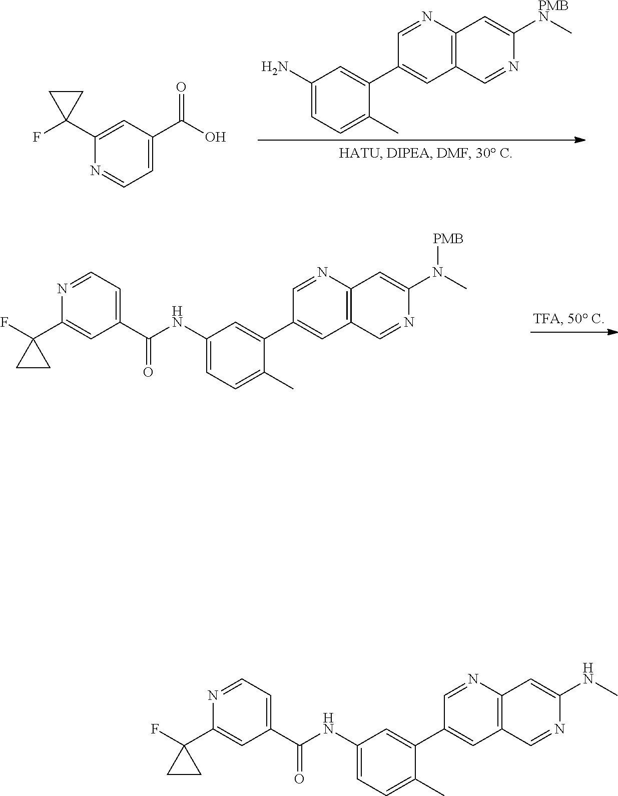

Step 1. To a solution of 2-(1-fluorocyclopropyl)isonicotinic acid (60 mg, 0.33 mmol), 3-(5-amino-2-methylphenyl)-N-(4-methoxybenzyl)-N-methyl-1,6-naphthyridin-7-amine (60 mg, 0.156 mmol) and 1-[bis(dimethylamino)methylene]-1H-1,2,3-triazolo[4,5-b]pyridinium 3-oxid hexafluorophosphate (89 mg, 0.234 mmol) in N,N-dimethylformamide (5 mL) was added N-ethyl-N-isopropylpropan-2-amine (101 mg, 0.78 mmol). The mixture was stirred at 30° C. for 1 hour. The reaction was quenched with water (60 mL) then extracted with ethyl acetate (60 mL×2). The combined organic layers were washed with brine (60 ML), dried over anhydrous sodium sulfate, and filtered. The filtrate was concentrated. The residue was purified by flash chromatography (silica, petroleum ether/ethyl acetate=1/1) to afford 2-(1-fluorocyclopropyl)-N-(3-(7-((4-methoxybenzyl)(methyl)amino)-1,6-naphthyridin-3-yl)-4-methylphenyl)isonicotinamide (80 mg, 0.146 mmol, 93%) as a yellow solid. MS (ESI) m/z 548.3 [M+H]⁺

Step 2. A solution of 2-(1-fluorocyclopropyl)-N-(3-(7-((4-methoxybenzyl)(methyl)amino)-1,6-naphthyridin-3-yl)-4-methylphenyl)isonicotinamide (80 mg, 0.146 mmol) in trifluoroacetic acid (6 mL) was stirred 50° C. for 1 hour. The mixture was concentrated and the residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(1-fluorocyclopropyl)-N-(4-methyl-3-(7-(methylamino)-1,6-naphthyridin-3-yl)phenyl)isonicotinamide (22.5 mg, 0.053 mmol, 36%) as a yellow solid. ¹H NMR (400 MHz, DMSO-d₆) δ 10.63 (s, 1H), 8.98 (s, 1H), 8.82 (d, J=2.0 Hz, 1H), 8.70 (d, J=4.8 Hz, 1H), 8.24 (d, J=1.6 Hz, 1H), 8.08 (s, 11H), 7.78-7.6 (m, 3H), 7.37 (d, J=5.2 Hz, 1H), 6.91 (d, J=5.2 Hz, 1H), 6.62 (s, 1H), 2.87 (d, J=5.2 Hz, 3H), 2.31 (d, J=1.6 Hz, 3H), 1.61-1.60 (m, 2H), 1.41-1.38 (m, 2H). MS (ESI) m/z 428.0 [M+H]⁺

### Example 19. Synthesis of 3-(Dimethylamino)-N-(6-methyl-5-(7-(methylamino)-1,6-naphthyridin-3-yl)pyridin-3-yl)benzamide (Compound 19)

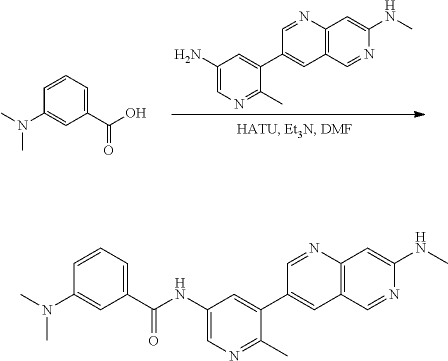

A mixture of 3-(dimethylamino)benzoic acid (23 mg, 0.14 mmol), 3-(5-amino-2-methylpyridin-3-yl)-N-methyl-1,6-naphthyridin-7-amine (37 mg, 0.14 mmol), 1-[bis(dimethylamino)methylene]-1H-1,2,3-triazolo[4,5-b]pyridinium 3-oxid hexafluorophosphate (160 mg, 0.42 mmol) and triethylamine (0.2 mL) in N,N-dimethylformamide (5 mL) was stirred at room temperature for 2 hours. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-(dimethylamino)-N-(6-methyl-5-(7-(methylamino)-1,6-naphthyridin-3-yl)pyridin-3-yl)benzamide (10.8 mg, 0.03 mmol, 19%) as a yellow solid. ¹H NMR (500 MHz, DMSO-d₆) δ 10.37 (s, 1H), 8.99 (s, 1H), 8.90 (d, J=2.4 Hz, 1H), 8.86 (d, J=2.3 Hz, 1H), 8.31 (d, J=2.1 Hz, 1H), 8.17 (d, J=2.4 Hz, 1H), 7.34 (t, J=8.0 Hz, 1H), 7.29-7.24 (m, 2H), 6.95 (dt, J=4.6, 3.4 Hz, 2H), 6.64 (s, 1H), 2.96 (d, J=7.0 Hz, 6H), 2.88 (d, J=5.0 Hz, 3H), 2.48 (s, 3H). MS (ESI) m/z 412.8 [M+H]⁺

### Example 20. Synthesis of N-(6-Methyl-5-(7-(methylamino)-1,6-naphthyridin-3-yl)pyridin-3-yl)-4-(trifluoromethyl)picolinamide (Compound 20)

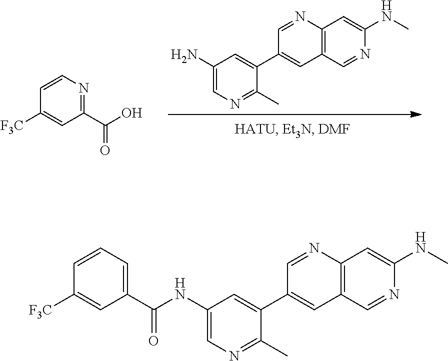 [SMILES: O=C(O)c1cc(C(F)(F)F)ccn1 ; CNc1cc2ncc(-c3cc(N)cnc3C)cc2cn1 ; CNc1cc2ncc(-c3cc(NC(=O)c4cccc(C(F)(F)F)c4)cnc3C)cc2cn1]

A mixture of 4-(trifluoromethyl)picolinic acid (36 mg, 0.19 mmol), 3-(5-amino-2-methylpyridin-3-yl)-N-methyl-1,6-naphthyridin-7-amine (50 mg, 0.19 mmol), 1-[bis(dimethylamino)methylene]-1H-1,2,3-triazolo[4,5-b]pyridinium 3-oxid hexafluorophosphate (217 mg, 0.57 mmol) and triethylamine (0.2 mL) in N,N-dimethylformamide (3 mL) was stirred at room temperature for 1 hour. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(6-methyl-5-(7-(methylamino)-1,6-naphthyridin-3-yl)pyridin-3-yl)-4-(trifluoromethyl)picolinamide (31.6 mg, 0.07 mmol, 38%) as a yellow solid. ¹H NMR (400 MHz, DMSO-d₆) δ 11.14 (s, 1H), 9.12-9.03 (m, 2H), 9.00 (s, 1H), 8.87 (d, J=2.3 Hz, 1H), 8.37 (s, 1H), 8.32 (dd, J=4.9, 2.2 Hz, 2H), 8.12 (d, J=4.0 Hz, 1H), 6.97 (q, J=4.9 Hz, 1H), 6.64 (s, 1H), 2.88 (d, J=5.0 Hz, 3H), 2.51 (d, J=1.4 Hz, 3H). MS (ESI) m/z 438.8 [M+H]⁺

### Example 21. Synthesis of N-(6-Methyl-5-(7-(methylamino)-1,6-naphthyridin-3-yl)pyridin-3-yl)-5-(trifluoromethyl)nicotinamide (Compound 21)

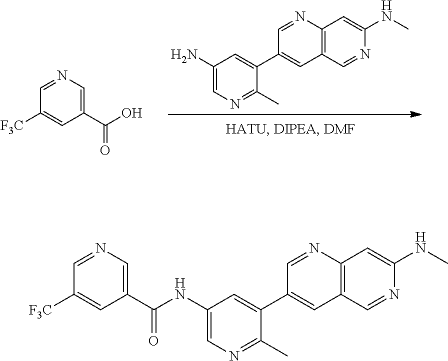 [SMILES: O=C(O)c1cncc(C(F)(F)F)c1 ; CNc1cc2ncc(-c3cc(N)cnc3C)cc2cn1 ; CNc1cc2ncc(-c3cc(NC(=O)c4cncc(C(F)(F)F)c4)cnc3C)cc2cn1]

A solution of 5-(trifluoromethyl)nicotinic acid (50 mg, 0.26 mmol), 3-(5-amino-2-methylpyridin-3-yl)-N-methyl-1,6-naphthyridin-7-amine (69 mg, 0.26 mmol), 1-[bis(dimethylamino)methylene]-1H-1,2,3-triazolo[4,5-b]pyridinium 3-oxid hexafluorophosphate (149 mg, 0.39 mmol) and N,N-diisopropylethylamine (68 mg, 0.52 mmol) in N,N-dimethylformamide (4 mL) was stirred for 2 hours at room temperature. The mixture was concentrated in vacuo. The residue was diluted with water (100 mL) and extracted with ethyl acetate (3×100 mL). The combined organic phases were concentrated in vacuo. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to yield N-(6-methyl-5-(7-(methylamino)-1,6-naphthyridin-3-yl) pyridin-3-yl)-2-(trifluoromethyl)isonicotinamide (20.9 mg, 15%) as a white solid. ¹H NMR (400 MHz, DMSO-d₆) δ 10.88 (s, 1H), 9.41 (d, J=1.7 Hz, 1H), 9.22 (d, J=1.2 Hz, 1H), 9.00 (s, 1H), 8.88 (dd, J=8.9, 2.4 Hz, 2H), 8.73 (s, 1N), 8.33 (d, J=2.0 Hz, 1H), 8.17 (d, J=2.4 Hz, 1H), 6.98 (q, J=4.9 Hz, 1H), 6.64 (s, 1H), 2.88 (d, J=4.9 Hz, 3H), 2.50 (s, 3H). MS (ESI) m/z 439.3 [M+H]⁺

### Example 22. Synthesis of 4-(Difluoromethyl)-N-(6-methyl-5-(7-(methylamino)-1,6-naphthyridin-3-yl)pyridin-3-yl)picolinamide (Compound 22)

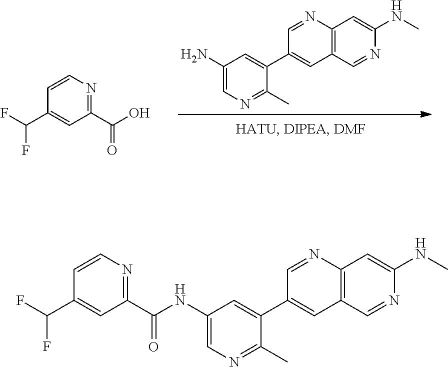 [SMILES: O=C(O)c1cc(C(F)F)ccn1 ; CNc1cc2ncc(-c3cc(N)cnc3C)cc2cn1 ; CNc1cc2ncc(-c3cc(NC(=O)c4cc(C(F)F)ccn4)cnc3C)cc2cn1]

To a solution of 4-(difluoromethyl)picolinic acid (20 mg, 0.11 mmol) in N,N-dimethylformamide (3 mL) were added 3-(5-amino-2-methylpyridin-3-yl)-N-methyl-1,6-naphthyridin-7-amine (29 mg, 0.11 mmol), 1-[bis(dimethylamino)methylene]-1H-1,2,3-triazolo[4,5-b]pyridinium 3-oxid hexafluorophosphate (84 mg, 0.22 mmol) and N,N-diisopropylethylamine (43 mg, 0.33 mmol) at room temperature. The reaction mixture was stirred at room temperature for 3 hours. The mixture was diluted with water (30 mL), and then extracted with ethyl acetate (3×15 mL). The combined organic layers were washed with brine (2×30 mL), dried over anhydrous sodium sulfate, filtered, and concentrated. The crude residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(difluoromethyl)-N-(6-methyl-5-(7-(methylamino)-1,6-naphthyridin-3-yl)pyridin-3-yl)picolinamide (4.9 mg, 0.01 mmol, 10%) as a yellow solid. ¹H NMR (400 MHz, DMSO-d₆) δ 11.08 (s, 1H), 9.05 (d, J=2.0 Hz, 1H), 8.99 (s, 1H), 8.95 (d, J=4.8 Hz, 1H), 8.87 (d, J=2.0 Hz, 1H), 8.33-8.29 (m, 3H), 7.90 (d, J=4.4 Hz, 1H), 7.40-7.12 (m, 1H), 6.98-6.96 (m, 11H), 6.63 (s, 1H), 2.88 (d, J=4.8 Hz, 3H), 2.50 (s, 3H). MS (ESI) m/z 421.2 [M+H]⁺

### Example 23. Synthesis of 4-(1,1-Difluoroethyl)-N-(6-methyl-5-(7-(methylamino)-1,6-naphthyridin-3-yl)pyridin-3-yl)picolinamide (Compound 23)

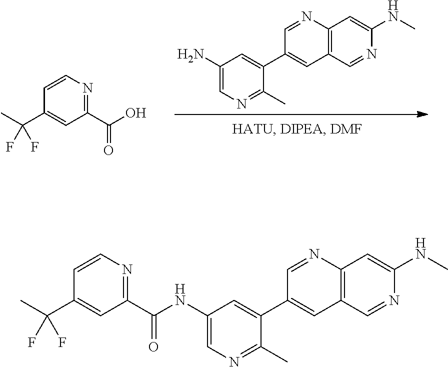 [SMILES: CC(F)(F)c1ccnc(C(=O)O)c1 ; CNc1cc2ncc(-c3cc(N)cnc3C)cc2cn1 ; CNc1cc2ncc(-c3cc(NC(=O)c4cc(C(C)(F)F)ccn4)cnc3C)cc2cn1]

A solution of 4-(1,1-difluoroethyl)picolinic acid (15 mg, 0.08 mmol), N,N-diisopropylethylamine (30 mg, 0.24 mmol), 1-[bis(dimethylamino)methylene]-1H-1,2,3-triazolo[4,5-b]pyridinium 3-oxid hexafluorophosphate (60 mg, 0.16 mmol) and 3-(5-amino-2-methylpyridin-3-yl)-N-methyl-1,6-naphthyridin-7-amine (21.2 mg, 0.08 mmol) in N,N-dimethylformamide (3 mL) was stirred at room temperature for 2 hours. The mixture was diluted with water (10 mL), and then extracted with ethyl acetate (20 mL×3). The combined organic layers were washed with brine (10 mL), dried over anhydrous sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(1,1-difluoroethyl)-N-(6-methyl-5-(7-(methylamino)-1,6-naphthyridin-3-yl)pyridin-3-yl)picolinamide (5.2 mg, 0.012 mmol, 15%) as a yellow solid. ¹H NMR (400 MHz, DMSO-d₆) δ 11.08 (s, 1H), 9.06 (d, J=2.4 Hz, 1H), 9.00 (s, 1H), 8.93 (d, J=5.0 Hz, 1H), 8.87 (d, J=2.3 Hz, 1H), 8.32 (dd, J=6.2, 2.1 Hz, 2H), 8.26 (s, 1H), 7.90 (d, J=5.0 Hz, 1H), 6.97 (d, J=5.0 Hz, 1H), 6.64 (s, 1H), 2.88 (d, J=4.9 Hz, 3H), 2.49-2.41 (m, 3H), 2.11-1.99 (m, 3H). MS (ESI) m/z 435.1[M+H]⁺

### Example 24. Synthesis of 2-(2-Fluoropropan-2-yl)-N-(6-methyl-5-(7-(methylamino)-1,6-naphthyridin-3-yl)pyridin-3-yl)isonicotinamide (Compound 24)

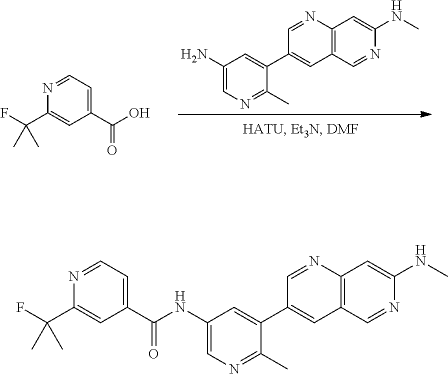 [SMILES: CC(C)(F)c1cc(C(=O)O)ccn1 ; CNc1cc2ncc(-c3cc(N)cnc3C)cc2cn1 ; CNc1cc2ncc(-c3cc(NC(=O)c4ccnc(C(C)(C)F)c4)cnc3C)cc2cn1]

A mixture of 2-(2-fluoropropan-2-yl)isonicotinic acid (35 mg, 0.19 mmol), 3-(5-amino-2-methylpyridin-3-yl)-N-methyl-1,6-naphthyridin-7-amine (50 mg, 0.19 mmol), 1-[bis(dimethylamino)methylene]-1H-1,2,3-triazolo[4,5-b]pyridinium 3-oxid hexafluorophosphate (217 mg, 0.57 mmol) and triethylamine (0.2 mL) in N,N-dimethylformamide (3 mL) was stirred at room temperature for 1 hour. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(2-fluoropropan-2-yl)-N-(6-methyl-5-(7-(methylamino)-1,6-naphthyridin-3-yl)pyridin-3-yl)isonicotinamide (14.2 mg, 0.03 mmol, 17%) as a yellow solid. ¹H NMR (400 MHz, DMSO-d₆) δ 10.82 (s, 1H), 9.00 (s, 1H), 8.90 (d, J=2.4 Hz, 1H), 8.87 (d, J=2.3 Hz, 1H), 8.79 (d, J=5.1 Hz, 1H), 8.32 (d, J=1.8 Hz, 1H), 8.17 (d, J=2.4 Hz, 1H), 8.07 (s, 1H), 7.86 (dd, J=5.1, 1.6 Hz, 1H), 7.00-6.93 (m, 1H), 6.64 (s, 1H), 2.87 (d, J=5.0 Hz, 3H), 2.51 (d, J=0.7 Hz, 3H), 1.74 (s, 3H), 1.69 (s, 3H). MS (ESI) m/z 430.8 [M+H]⁺

### Example 25. Synthesis of 5-Methyl-N-(6-methyl-5-(7-(methylamino)-1,6-naphthyridin-3-yl)pyridin-3-yl)-4-(trifluoromethyl)picolinamide (Compound 25)

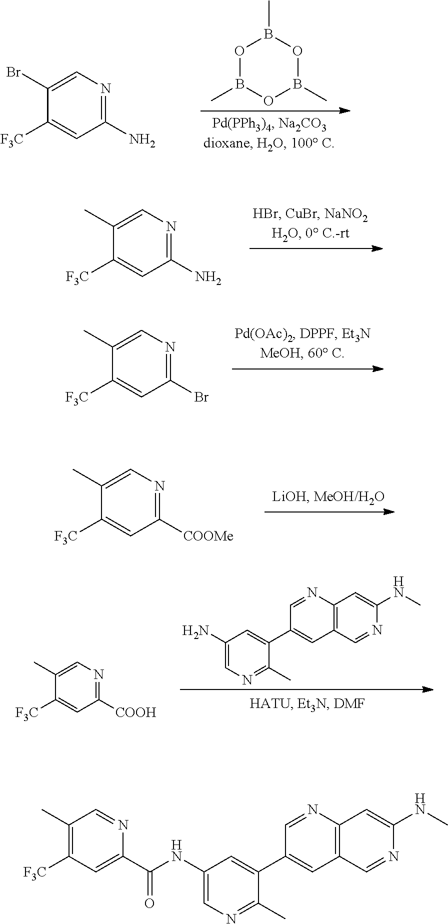

Step 1. A mixture of 5-bromo-4-(trifluoromethyl)pyridin-2-amine (1.00 g, 4.17 mmol), 2,4,6-trimethyl-1,3,5,2,4,6-trioxatriborinane (3.5 M in tetrahydrofuran, 3.6 mL, 12.45 mmol), tetrakis(triphenylphosphine)palladium (481 mg, 0.42 mmol) and sodium carbonate (883 mg, 8.33 mmol) in water (3 mL) and dioxane (10 mL) was stirred at 100° C. for 18 hours under argon. The resulting mixture was cooled to room temperature and concentrated. The residue was purified by flash chromatography (silica, petroleum ether/ethyl acetate=2/1) to afford 5-methyl-4-(trifluoromethyl)pyridin-2-amine (700 mg, 3.98 mmol, 95%) as a yellow oil. MS (ESI) m/z 177.1 [M+H]⁺

Step 2. To a solution of 5-methyl-4-(trifluoromethyl)pyridin-2-amine (700 mg, 3.98 mmol) in hydrogen bromide (10 mL, 40% in water) was added sodium nitrite (549 mg, 7.95 mmol) at 0° C. The mixture was stirred at 0° C. for 1 hour and cuprous bromide (1.14 g, 7.95 mmol) in hydrogen bromide (2 mL, 40% in water) was added. The reaction mixture was stirred at room temperature for 18 hours. The reaction was quenched by aqueous sodium hydroxide (1 M in water) until pH=9 and extracted with ethyl acetate (50 mL×3). The combined organic phases were concentrated and purified by flash chromatography (silica, petroleum ether/ethyl acetate=10/1) to afford 2-bromo-5-methyl-4-(trifluoromethyl)pyridine (210 mg, 0.88 mmol, 22%) as a yellow oil. MS (EST) m/z 239.9 [M+H]⁺

Step 3. A solution of 2-bromo-5-methyl-4-(trifluoromethyl)pyridine (210 mg, 0.88 mmol), 1,1′-bis(diphenylphosphino)ferrocene (97 mg, 0.18 mmol), palladium (II) acetate (20 mg, 0.09 mmol) and triethylamine (266 mg, 2.64 mmol) in methanol (5 mL) was stirred at 60° C. for 18 hours under carbon monoxide atmosphere. The reaction was cooled to room temperature and used without further purification. MS (ESI) m/z 220.1 [M+H]⁺

Step 4. The mixture of 5-methyl-4-(trifluoromethyl)picolinic acid (150 mg, 0.73 mmol) and lithium hydroxide (88 mg, 3.66 mmol) in methanol (5 mL) and water (2 mL) was stirred at room temperature for 1 hour. The reaction mixture was adjusted to pH=4 with 1 N hydrochloric acid and concentrated. The residue was purified by flash chromatography (silica, dichloromethane/methanol=10/1) to afford 5-methyl-4-(trifluoromethyl)picolinic acid (60 mg, 0.29 mmol, 33% over two steps) as a yellow solid. MS (ESI) m/z 205.8 [M+H]⁺

Step 5. A mixture of 5-methyl-4-(trifluoromethyl)picolinic acid (30 mg, 0.15 mmol), 3-(5-amino-2-methylpyridin-3-yl)-N-methyl-1,6-naphthyridin-7-amine (39 mg, 0.15 mmol), 1-[bis(dimethylamino)methylene]-1H-1,2,3-triazolo[4,5-b]pyridinium 3-oxid hexafluorophosphate (67 mg, 0.18 mmol) and triethylamine (44 mg, 0.44 mmol) in N,N-dimethylformamide (2 mL) was stirred at room temperature for 2 hours. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 5-methyl-N-(6-methyl-5-(7-(methylamino)-1,6-naphthyridin-3-yl)pyridin-3-yl)-4-(trifluoromethyl)picolinamide (15.8 mg, 0.035 mmol, 23%) as a yellow solid. ¹H NMR (400 MHz, DMSO-d₆) δ 11.08 (s, 1H), 9.11-8.97 (m, 2H), 8.94-8.83 (m, 2H), 8.38-8.22 (m, 3H), 7.02-6.91 (m, 1H), 6.64 (s, 1H), 2.88 (d, J=5.0 Hz, 3H), 2.58 (s, 3H), 2.50 (s, 3H). MS (EST) m/z 453.1 [M+H]⁺

### Example 26. Synthesis of 3-Chloro-N-(6-methyl-5-(7-(methylamino)-1,6-naphthyridin-3-yl)pyridin-3-yl)-4-(trifluoromethyl)picolinamide (Compound 26)

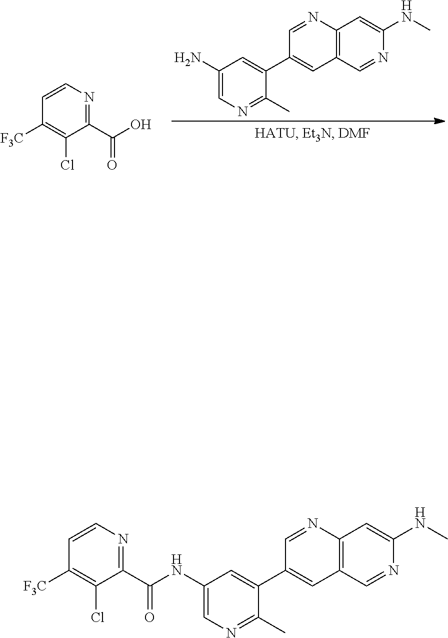 [SMILES: O=C(O)c1nccc(C(F)(F)F)c1Cl ; CNc1cc2ncc(-c3cc(N)cnc3C)cc2cn1 ; CNc1cc2ncc(-c3cc(NC(=O)c4nccc(C(F)(F)F)c4Cl)cnc3C)cc2cn1]

A solution of 3-chloro-4-(trifluoromethyl)picolinic acid (0.050 g, 0.22 mmol), 3-(5-amino-2-methylpyridin-3-yl)-N-methyl-1,6-naphthyridin-7-amine (0.059 g, 0.22 mmol), 1-[bis(dimethylamino)methylene]-1H-1,2,3-triazolo[4,5-b]pyridinium 3-oxid hexafluorophosphate (0.13 g, 0.33 mmol) and triethylamine (0.079 g, 0.78 mmol) in dichloromethane (5.0 mL) was stirred at room temperature for 16 hours. The reaction mixture was quenched with water (20.0 mL) and extracted with dichloromethane (20 mL×2). The combined organic layers were washed with brine (10 mL), dried over anhydrous sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 3-chloro-N-(6-methyl-5-(7-(methylamino)-1,6-naphthyridin-3-yl)pyridin-3-yl)-4-(trifluoromethyl)picolinamide (14.5 mg, 0.03 mmol, 13%) as a yellow solid. ¹H NMR (400 MHz, CD₃OD): δ 8.95 (s, 1H), 8.85-8.81 (m, 3H), 8.35 (s, 1H), 8.23 (s, 1H), 7.96 (s, 1H), 8.71 (s, 1H), 2.98 (s, 3H), 2.55 (s, 3H). MS (ESI) m/z 473.1 [M+H]⁺

### Example 27. Synthesis of N-(6-Methyl-5-(7-(methylamino)-1,6-naphthyridin-3-yl)pyridin-3-yl)-2-(trifluoromethyl)isonicotinamide (Compound 27)

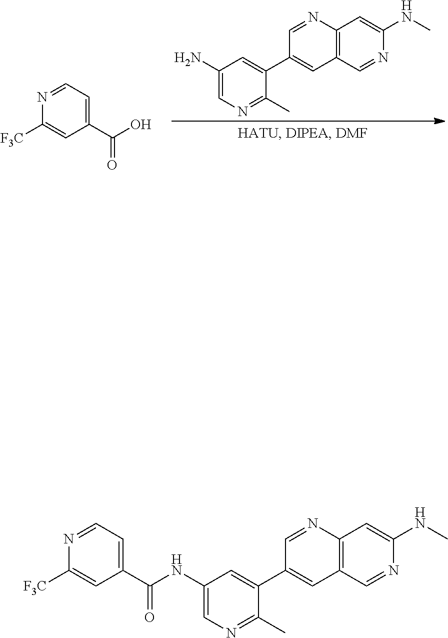 [SMILES: O=C(O)c1ccnc(C(F)(F)F)c1 ; CNc1cc2ncc(-c3cc(N)cnc3C)cc2cn1 ; CNc1cc2ncc(-c3cc(NC(=O)c4ccnc(C(F)(F)F)c4)cnc3C)cc2cn1]

A solution of 2-(trifluoromethyl)isonicotinic acid (50 mg, 0.26 mmol), 3-(5-amino-2-methylpyridin-3-yl)-N-methyl-1,6-naphthyridin-7-amine (69 mg, 0.26 mmol), 1-[bis(dimethylamino)methylene]-1H-1,2,3-triazolo[4,5-b]pyridinium 3-oxid hexafluorophosphate (149 mg, 0.39 mmol) and N,N-diisopropylethylamine (68 mg, 0.52 mmol) in N,N-dimethylformamide (4 mL) was stirred at room temperature for 2 hours. The mixture was concentrated in vacuo. The residue was diluted with water (100 mL) and extracted with ethyl acetate (3×100 mL). The organic phase was concentrated and purified by flash chromatography (silica, petroleum ether/ethyl acetate=%) to afford N-(6-methyl-5-(7-(methylamino)-1,6-naphthyridin-3-yl)pyridin-3-yl)-2-(trifluoromethyl)isonicotinamide (17.6 mg, 0.040 mmol, 15%) as a white solid. ¹H NMR (400 MHz, DMSO-d₆) δ 10.94 (s, 1H), 9.02 (d, J=5.0 Hz, 1H), 9.00 (s, 1H), 8.88 (dd, J=14.3, 2.4 Hz, 2H), 8.41 (s, 1H), 8.32 (d, J=2.2 Hz, 1H), 8.23 (d, J=5.8 Hz, 1H), 8.16 (d, J=2.4 Hz, 1H), 6.96 (q, J=4.7 Hz, 1H), 6.64 (s, 1H), 2.88 (d, J=5.0 Hz, 3H), 2.50 (s, 3H). MS (ESI) m/z 439.3 [M+H]⁺

### Example 28. Synthesis of 2-(Difluoromethyl)-N-(6-methyl-5-(7-(methylamino)-1,6-naphthyridin-3-yl)pyridin-3-yl)isonicotinamide (Compound 28)

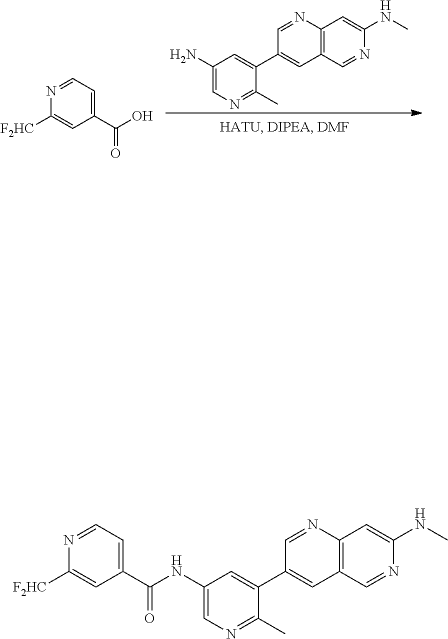 [SMILES: O=C(O)c1ccnc(C(F)F)c1 ; CNc1cc2ncc(-c3cc(N)cnc3C)cc2cn1 ; CNc1cc2ncc(-c3cc(NC(=O)c4ccnc(C(F)F)c4)cnc3C)cc2cn1]

A solution of 2-(difluoromethyl)isonicotinic acid (30 mg, 0.17 mmol), N,N-diisopropylethylamine (67 mg, 0.52 mmol), 1-[bis(dimethylamino)methylene]-1H-1,2,3-triazolo[4,5-b]pyridinium 3-oxid hexafluorophosphate (152 mg, 0.34 mmol) and 3-(5-amino-2-methylpyridin-3-yl)-N-methyl-1,6-naphthyridin-7-amine (21.2 mg, 0.08 mmol) in N,N-dimethylformamide (3 mL) was stirred at room temperature for 2 hours. The mixture was diluted with water (10 mL), and then extracted with ethyl acetate (20 mL×3). The combined organic layers were washed with brine (10 mL), dried over anhydrous sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(difluoromethyl)-N-(6-methyl-5-(7-(methylamino)-1,6-naphthyridin-3-yl)pyridin-3-yl)isonicotinamide (27.1 mg, 0.064 mmol, 80%) as a yellow solid. ¹H NMR (400 MHz, DMSO-d₆) δ 10.95 (s, 2H), 9.00 (s, 2H), 8.94 (d, J=5.1 Hz, 2H), 8.91 (d, J=2.3 Hz, 2H), 8.87 (d, J=2.3 Hz, 2H), 8.33 (d, J=2.0 Hz, 2H), 8.23 (s, 2H), 8.18 (d, J=2.3 Hz, 2H), 8.11 (d, J=4.8 Hz, 2H), 7.28-6.93 (m, 4H), 6.64 (s, 2H), 2.87 (d, J=5.0 Hz, 7H), 2.51 (s, 7H). MS (ESI) m/z 421.2 [M+H]⁺

### Example 29. Synthesis of 2-(1,1-Difluoroethyl)-N-(6-methyl-5-(7-(methylamino)-1,6-naphthyridin-3-yl)pyridin-3-yl)isonicotinamide (Compound 29)

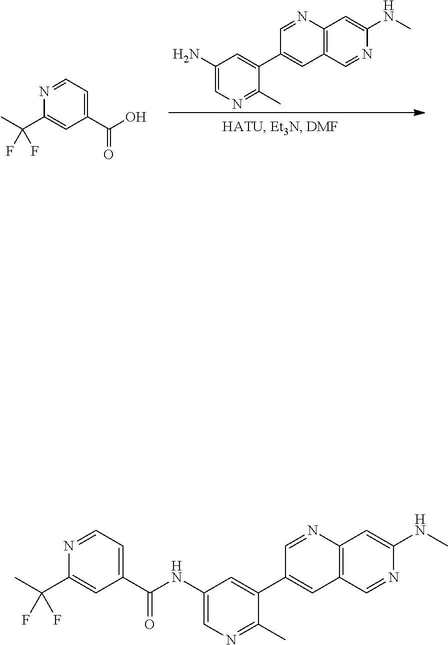 [SMILES: CC(F)(F)c1cc(C(=O)O)ccn1 ; CNc1cc2ncc(-c3cc(N)cnc3C)cc2cn1 ; CNc1cc2ncc(-c3cc(NC(=O)c4ccnc(C(C)(F)F)c4)cnc3C)cc2cn1]

A mixture of 2-(1,1-difluoroethyl)isonicotinic acid (28 mg, 0.15 mmol), 3-(5-amino-2-methylpyridin-3-yl)-N-methyl-1,6-naphthyridin-7-amine (40 mg, 0.15 mmol), 1-[bis(dimethylamino)methylene]-1H-1,2,3-triazolo[4,5-b]pyridinium 3-oxid hexafluorophosphate (171 mg, 0.45 mmol) and triethylamine (0.2 mL) in N,N-dimethylformamide (3 mL) was stirred at room temperature for 1 hour. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 300%-70% in 15 min) to afford 2-(1,1-difluoroethyl)-N-(6-methyl-5-(7-(methylamino)-1,6-naphthyridin-3-yl)pyridin-3-yl)isonicotinamide (8.4 mg, 0.02 mmol, 13%) as a yellow solid. ¹H NMR (500 MHz, DMSO-d₆) δ 10.89 (s, 1H), 9.00 (s, 1H), 8.93-8.89 (m, 2H), 8.87 (d, J=2.3 Hz, 1H), 8.32 (d, J=2.2 Hz, 1H), 8.23 (s, 1H), 8.17 (d, J=2.4 Hz, 1H), 8.06 (d, J=5.0 Hz, 1H), 6.96 (q, J=4.9 Hz, 1H), 6.64 (s, 1H), 2.88 (d, J=5.0 Hz, 3H), 2.50 (s, 3H), 2.06 (t, J=19.1 Hz, 3H). MS (ESI) m/z 434.9 [M+H]⁺

### Example 30. Synthesis of 4-(2-Cyanopropan-2-yl)-N-(6-methyl-5-(7-(methylamino)-1,6-naphthyridin-3-yl)pyridin-3-yl)picolinamide (Compound 30)

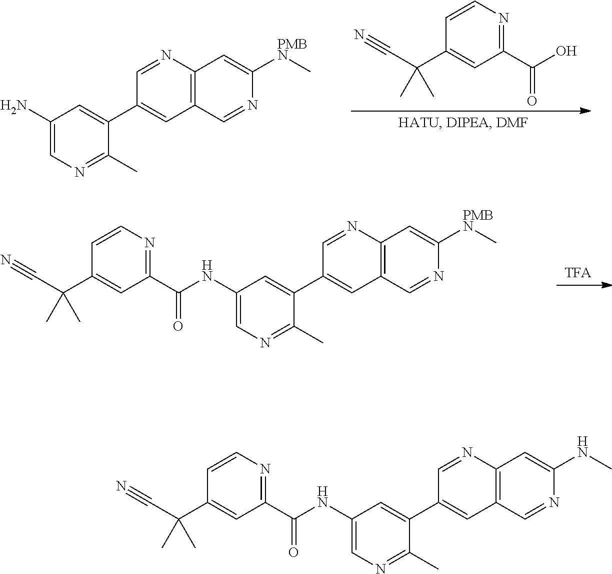

Step 1. To a solution of 4-(2-cyanopropan-2-yl)picolinic acid (35 mg, 0.18 mmol) in N,N-dimethylformamide (2 mL) were added 3-(5-amino-2-methylpyridin-3-yl)-N-(4-methoxybenzyl)-N-methyl-1,6-naphthyridin-7-amine (70 mg, 0.18 mmol), 1-[bis(dimethylamino)methylene]-1H-1,2,3-triazolo[4,5-b]pyridinium 3-oxid hexafluorophosphate (75 mg, 0.20 mmol) and N,N-diisopropylethylamine (46 mg, 0.36 mmol) at room temperature. The reaction mixture was stirred at room temperature for 1 hour. The mixture was diluted with water (10 mL), and then extracted with ethyl acetate (10 mL×2). The combined organic layers were washed with water (10 mL×3) and brine (10 mL), dried over anhydrous sodium sulfate, filtered, and concentrated. The crude residue was purified by flash chromatography (silica, ethyl acetate) to afford 4-(2-cyanopropan-2-yl)-N-(5-(7-((4-methoxybenzyl)(methyl)amino)-1,6-naphthyridin-3-yl)-6-methylpyridin-3-yl)picolinamide (60 mg, 0.11 mmol, 60%) as a yellow solid. MS (ESI) m/z 558.0 [M+H]⁺

Step 2. A solution of 4-(2-cyanopropan-2-yl)-N-(5-(7-((4-methoxybenzyl)(methyl)amino)-1,6-naphthyridin-3-yl)-6-methylpyridin-3-yl)picolinamide (60 mg, 0.11 mmol) in trifluoroacetic acid (4 mL) was stirred at room temperature for 2 hours. Then the reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(6-methyl-5-(7-(methylamino)-1,6-naphthyridin-3-yl)pyridin-3-yl)picolinamide (35.4 mg, 0.081 mmol, 74%) as a yellow solid. ¹H NMR (500 MHz, DMSO-d₆) δ 11.02 (s, 1H), 9.06 (d, J=3.0 Hz, 1H), 8.99 (s, 1H), 8.87 (d, J=2.0 Hz, 1H), 8.82 (d, J=5.5 Hz, 1H), 8.31-8.29 (m, 3H), 7.86 (dd, J=5.0 Hz, 1.5 Hz, 1H), 8.95-8.94 (m, 1H), 6.64 (s, 1H), 2.88 (d, J=4.5 Hz, 3H), 2.50 (s, 3H), 1.77 (s, 6H). MS (ESI) m/z 437.8 [M+H]⁺

### Example 31. Synthesis of 4-(1-Cyanocyclobutyl)-N-(6-methyl-5-(7-(methylamino)-1,6-naphthyridin-3-yl)pyridin-3-yl)picolinamide (Compound 31)

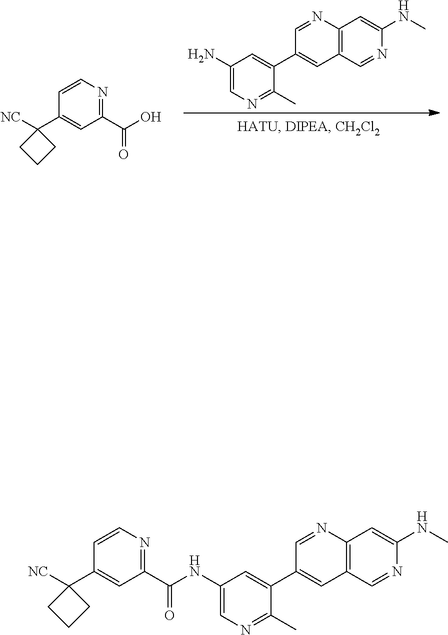 [SMILES: N#CC1(c2ccnc(C(=O)O)c2)CCC1 ; CNc1cc2ncc(-c3cc(N)cnc3C)cc2cn1 ; CNc1cc2ncc(-c3cc(NC(=O)c4cc(C5(C#N)CCC5)ccn4)cnc3C)cc2cn1]

A mixture of 4-(1-cyanocyclobutyl)picolinic acid (45 mg, 0.22 mmol), 3-(5-amino-2-methylpyridin-3-yl)-N-methyl-1,6-naphthyridin-7-amine (50 mg, 0.189 mmol), 1-[bis(dimethylamino)methylene]-1H-1,2,3-triazolo[4,5-b]pyridinium 3-oxid hexafluorophosphate (107 mg, 0.28 mmol) and N,N-diisopropylethylamine (73 mg, 0.56 mmol) in dichloromethane (10 mL) was stirred at room temperature for 1 hour. The reaction was quenched with water and extracted with dichloromethane (10 mL×3). The combined organic layers were washed with brine (20 mL), dried over sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(1-cyanocyclobutyl)-N-(6-methyl-5-(7-(methylamino)-1,6-naphthyridin-3-yl)pyridin-3-yl)picolinamide (43.8 mg, 0.097 mmol, 44%) as a yellow solid. ¹H NMR (400 MHz, DMSO-d₆) δ 11.05 (s, 1H), 9.06 (d, J=2.4 Hz, 1H), 9.00 (s, 1H), 8.87 (d, J=2.4 Hz, 1H), 8.84 (dd, J=5.1, 0.6 Hz, 1H), 8.32 (dd, J=4.1, 2.4 Hz, 2H), 8.29-8.14 (m, 1H), 7.86 (dd, J=5.1, 2.0 Hz, 1H), 6.98 (d, J=5.0 Hz, 1H), 6.64 (s, 1H), 2.88 (d, J=4.9 Hz, 3H), 2.85-0.79 (m, 2H), 2.72 (ddd, J=10.7, 9.7, 5.6 Hz, 2H), 2.51 (d, 3H), 2.40-2.26 (m, 1H), 2.16-2.02 (m, 1H). MS (ESI) m/z 450.1 [M+H]⁺

### Example 32. Synthesis of 4-(1-Cyanocyclopropyl)-N-(6-methyl-5-(7-(methylamino)-1,6-naphthyridin-3-yl)pyridin-3-yl)picolinamide (Compound 32)

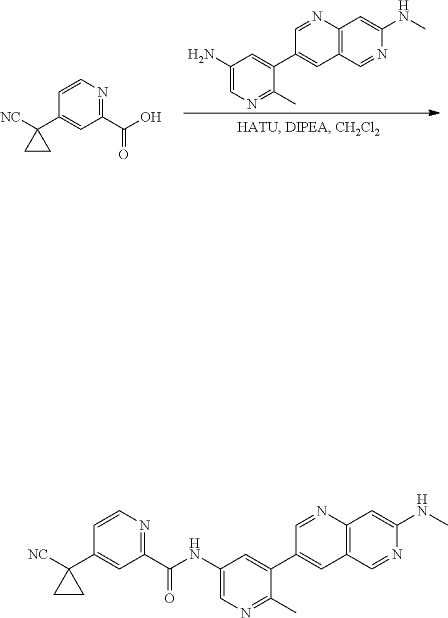 [SMILES: N#CC1(c2ccnc(C(=O)O)c2)CC1 ; CNc1cc2ncc(-c3cc(N)cnc3C)cc2cn1 ; CNc1cc2ncc(-c3cc(NC(=O)c4cc(C5(C#N)CC5)ccn4)cnc3C)cc2cn1]

A mixture of 4-(1-cyanocyclopropyl)picolinic acid (42 mg, 0.22 mmol), 3-(5-amino-2-methylpyridin-3-yl)-N-methyl-1,6-naphthyridin-7-amine (50 mg, 0.189 mmol), 1-[bis(dimethylamino)methylene]-1H-1,2,3-triazolo[4,5-b]pyridinium 3-oxid hexafluorophosphate (107 mg, 0.28 mmol) and N,N-diisopropylethylamine (73 mg, 0.56 mmol) in dichloromethane (10 mL) was stirred at room temperature for 1 hour. The reaction was quenched with water and extracted with dichloromethane (10 mL×3). The combined organic layers were washed with brine (20 mL), dried over sodium sulfate, filtered, and concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(1-cyanocyclopropyl)-N-(6-methyl-5-(7-(methylamino)-1,6-naphthyridin-3-yl)pyridin-3-yl)picolinamide (30.7 mg, 0.07 mmol, 32%) as a yellow solid. ¹H NMR (400 MHz, DMSO-d₆) δ 11.02 (s, 1H), 9.17-8.95 (m, 2H), 8.87 (d, J=2.2 Hz, 1H), 8.73 (d, J=5.2 Hz, 1H), 8.33 (d, J=5.0 Hz, 2H), 8.08 (d, J=1.8 Hz, 1H), 7.56 (dd, J=5.2, 2.0 Hz, 1H), 6.99 (s, 1H), 6.64 (s, 1H), 2.88 (d, J=4.7 Hz, 3H), 2.51 (d, J=1.6 Hz, 3H), 2.01 (dd, J=8.1, 5.1 Hz, 2H), 1.79 (dd, J=8.4, 5.4 Hz, 2H). MS (ESI) m/z 436.2 [M+H]⁺

### Example 33. Synthesis of 2-(1-Fluorocyclopropyl)-N-(6-methyl-5-(7-(methylamino)-1,6-naphthyridin-3-yl)pyridin-3-yl)isonicotinamide (Compound 33)

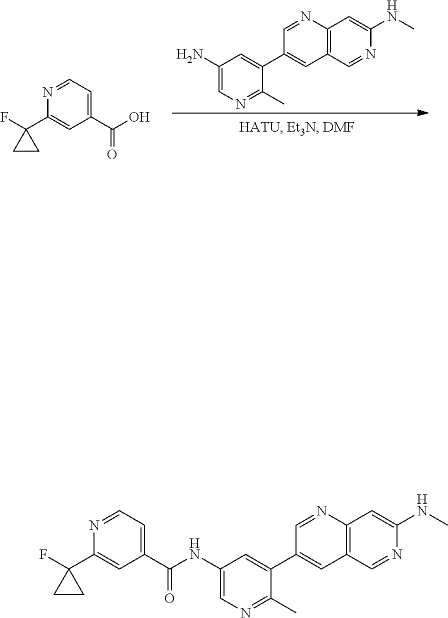 [SMILES: O=C(O)c1ccnc(C2(F)CC2)c1 ; CNc1cc2ncc(-c3cc(N)cnc3C)cc2cn1 ; CNc1cc2ncc(-c3cc(NC(=O)c4ccnc(C5(F)CC5)c4)cnc3C)cc2cn1]

A mixture of 2-(1-fluorocyclopropyl)isonicotinic acid (34 mg, 0.19 mmol), 3-(5-amino-2-methylpyridin-3-yl)-N-methyl-1,6-naphthyridin-7-amine (50 mg, 0.19 mmol), 1-[bis(dimethylamino)methylene]-1H-1,2,3-triazolo[4,5-b]pyridinium 3-oxid hexafluorophosphate (217 mg, 0.57 mmol) and triethylamine (0.3 mL) in N,N-dimethylformamide (5 mL) was stirred at room temperature for 2 hours. The resulting mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(1-fluorocyclopropyl)-N-(6-methyl-5-(7-(methylamino)-1,6-naphthyridin-3-yl)pyridin-3-yl)isonicotinamide (21.3 mg, 0.05 mmol, 26%) as a yellow solid. ¹H NMR (500 MHz, DMSO-d₆) δ 10.84 (s, 1H), 9.00 (s, 1H), 8.91 (s, 1H), 8.87 (s, 1H), 8.73 (d, J=4.8 Hz, 1H), 8.33 (s, 1H), 8.19 (s, 1H), 8.12 (s, 1H), 7.80 (d, J=4.9 Hz, 1H), 6.97 (d, J=4.9 Hz, 1H), 6.64 (s, 1H), 2.88 (d, J=4.6 Hz, 3H), 2.50-2.45 (m, 3H), 1.64-1.56 (m, 2H), 1.41 (q, J=8.5 Hz, 2H). MS (ESI) m/z 428.9 [M+H]⁺

### Example 34. Synthesis of 2-(3,3-Difluorocyclobutyl)-N-(6-methyl-5-(7-(methylamino)-1,6-naphthyridin-3-yl)pyridin-3-yl)isonicotinamide (Compound 34)

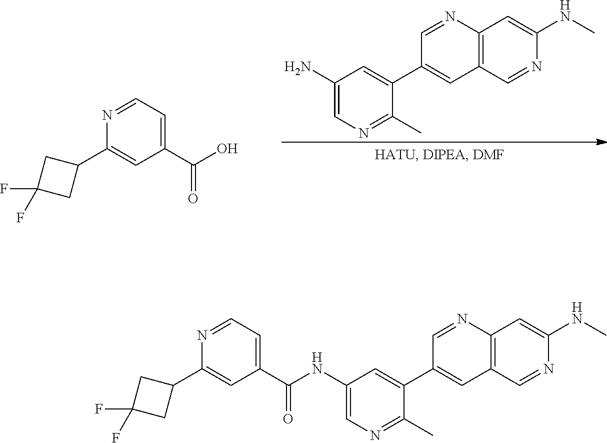 [SMILES: O=C(O)c1ccnc(C2CC(F)(F)C2)c1 ; CNc1cc2ncc(-c3cc(N)cnc3C)cc2cn1 ; CNc1cc2ncc(-c3cc(NC(=O)c4ccnc(C5CC(F)(F)C5)c4)cnc3C)cc2cn1]

To a solution of 3-(5-amino-2-methylpyridin-3-yl)-N-methyl-1,6-naphthyridin-7-amine (37 mg, 0.14 mmol) in N,N-dimethylformamide (4 mL) was added N,N-diisopropylethylamine (55 mg, 0.42 mmol), 1-[bis(dimethylamino)methylene]-1H-1,2,3-triazolo[4,5-b]pyridinium 3-oxid hexafluorophosphate (80 mg, 0.21 mmol) and 2-(3,3-difluorocyclobutyl)isonicotinic acid (30 mg, 0.14 mmol) at room temperature. The mixture was allowed to stir at room temperature for 1 hour.

The mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 2-(3,3-difluorocyclobutyl)-N-(6-methyl-5-(7-(methylamino)-1,6-naphthyridin-3-yl)pyridin-3-yl)isonicotinamide (10.7 mg, 0.02 mmol, 17%) as a yellow solid. ¹H NMR (400 MHz, CDCl₃) δ 8.87 (d, J=6.4 Hz, 2H), 8.80 (d, J=5.2 Hz, 1H), 8.68 (s, 1H), 8.31 (s, 1H), 8.06 (dd, J₁=2.0 Hz, J₂=13.2 Hz, 2H), 7.65 (s, 1H), 7.58 (d, J=5.2 Hz, 1H), 6.78 (s, 1H), 5.04 (s, 1H), 3.54-3.59 (m, 1H), 2.95-3.05 (m, 7H), 2.59 (s, 3H). MS (ESI) m/z 461.4 [M+H]⁺

### Examples 35 and 36. Synthesis of N-(6-Methyl-5-(7-(methylamino)-1,6-naphthyridin-3-yl)pyridin-3-yl)-2-((2S,4R)-4-(trifluoromethyl)tetrahydro-2H-pyran-2-yl)acetamide and N-(6-Methyl-5-(7-(methylamino)-1,6-naphthyridin-3-yl)pyridin-3-yl)-2-((2R,4R)-4-(trifluoromethyl)tetrahydro-2H-pyran-2-yl)acetamide (Compound 35 and Compound 36)

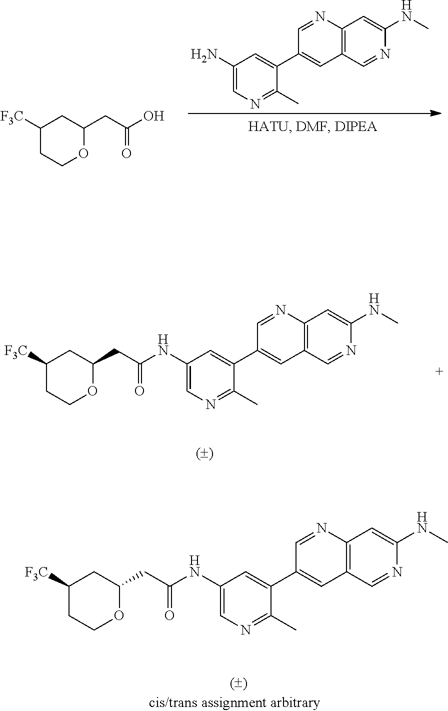 [SMILES: CNc1cc2ncc(-c3cc(N)cnc3C)cc2cn1 ; O=C(O)CC1CC(C(F)(F)F)CCO1 ; CNc1cc2ncc(-c3cc(NC(=O)C[C@@H]4C[C@H](C(F)(F)F)CCO4)cnc3C)cc2cn1 ; CNc1cc2ncc(-c3cc(NC(=O)C[C@H]4C[C@H](C(F)(F)F)CCO4)cnc3C)cc2cn1]

To a solution of 3-(5-amino-2-methylpyridin-3-yl)-N-methyl-1,6-naphthyridin-7-amine (125 mg, 0.47 mmol) in N,N-dimethylformamide (4 mL) was added N,N-diisopropylethylamine (183 mg, 1.42 mmol), 1-[bis(dimethylamino)methylene]-1H-1,2,3-triazolo[4,5-b]pyridinium 3-oxid hexafluorophosphate (269 mg, 0.71 mmol) and 2-(4-(trifluoromethyl)tetrahydro-2H-pyran-2-yl)acetic acid (100 mg, 0.47 mmol) at room temperature. The mixture was allowed to stir at room temperature for 1 hour. The mixture was purified by prep-HPLC (Column: Welch Xtimate 21.2×250 mm C18, 10 μm, Mobile Phase: A: water (10 mmol/L ammonium bicarbonate) B: acetonitrile; B %: 30-70% in 15 min) to afford the first eluting diastereomer (retention time 6.6 min) which was arbitrarily assigned as N-(6-methyl-5-(7-(methylamino)-1,6-naphthyridin-3-yl)pyridin-3-yl)-2-((2R,4R)-4-(trifluoromethyl)tetrahydro-2H-pyran-2-yl)acetamide (5.4 mg, 0.012 mmol, 3%) as a yellow solid. ¹H NMR (400 MHz, DMSO-d₆) δ 10.25 (s, 1H), 8.98 (s, 1H), 8.81 (d, J=2.4 Hz, 1H), 8.64 (d, J=2.4 Hz, 1H), 8.27 (d, J=2.0 Hz, 1H), 8.03 (d, J=2.4 Hz, 1H), 6.94 (q, J=4.4 Hz, 1H), 6.62 (s, 1H), 4.18 (s, 1H), 3.71-3.76 (m, 1H), 3.59 (s, 1H), 2.86 (d, J=5.2 Hz, 3H), 2.71 (s, 1H), 2.59-2.67 (m, 2H), 2.43 (s, 3H), 1.69-1.80 (in, 4H). MS (ESI) m/z 460.1 [M+H]⁺;
and the second eluting diastereomer (retention time 7.1 min) which was arbitrarily assigned as N-(6-methyl-5-(7-(methylamino)-1,6-naphthyridin-3-yl)pyridin-3-yl)-2-((2S,4R)-4-(trifluoromethyl)tetrahydro-2H-pyran-2-yl)acetamide (22.6 mg, 0.049 mmol, 10%) as a yellow solid. ¹H NMR (400 MHz, DMSO-d₆) δ 10.25 (s, 11H), 8.98 (s, 1H), 8.81 (d, J=2.4 Hz, 1H), 8.65 (d, J=2.4 Hz, 1H), 8.27 (d, J=1.6 Hz, 1H), 8.04 (d, J=2.0 Hz, 1H), 6.62 (s, 1H), 3.92-3.96 (m, 1H), 3.37-3.43 (in, 11H), 3.18-3.84 (m, 1H), 2.86 (d, J=4.8 Hz, 3H), 2.66-2.72 (m, 1H), 2.54 (d, J=6.0 Hz, 2H), 2.43 (s, 3H), 1.87 (d, J=13.2 Hz, 1H), 1.69 (d, J=12.8 Hz, 1H), 1.35-146 (m, 1H), 1.20-1.29 (m, 1H). MS (ESI) m/z 460.1 [M+H]⁺

### Example 37. Synthesis of N-(4-Chloro-2-fluoro-5-(7-(methylamino)-1,6-naphthyridin-3-yl)phenyl)-4-(2-cyanopropan-2-yl)picolinamide (Compound 37)

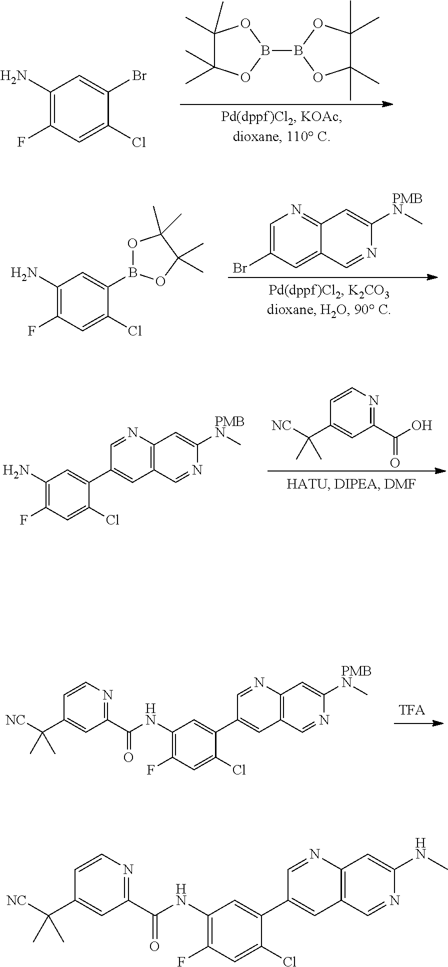

Step 1. A mixture of [1,1′-bis(diphenylphosphino)ferrocene]dichloropalladium(II) (73 mg, 0.1 mmol), 5-bromo-4-chloro-2-fluoroaniline (224 mg, 1 mmol), 4,4,4′,4′,5,5,5′,5′-octamethyl-2,2′-bi(1,3,2-dioxaborolane) (304 mg, 1.2 mmol) and potassium acetate (490 mg, 5 mmol) in dioxane (10 mL) was stirred at 110° C. for 12 hours under nitrogen. After cooling to room temperature, the reaction solution was concentrated and the residue was purified by flash chromatography (silica, dichloromethane/methanol=20/1) to afford 4-chloro-2-fluoro-5-(4,4,5,5-tetramethyl-1,3,2-dioxaborolan-2-yl)aniline (168 mg, 0.619 mmol, 62%) as a white solid. ¹H NMR (400 MHz, CDCl₃) δ=7.13 (d, J=10.0 Hz, 1H), 7.01 (d, J=11.2 Hz, 1H), 3.67 (brs, 2H), 1.35 (s, 12H). MS (ESI) m/z 272.2 [M+H]⁺

Step 2. A mixture of [1,1′-bis(diphenylphosphino)ferrocene]dichloropalladium(II) (21 mg, 0.029 mmol), 3-bromo-N-(4-methoxybenzyl)-N-methyl-1,6-naphthyridin-7-amine (200 mg, 0.558 mmol), 4-chloro-2-fluoro-5-(4,4,5,5-tetramethyl-1,3,2-dioxaborolan-2-yl)aniline (167 mg, 0.615 mmol) and potassium carbonate (231 mg, 1.671 mmol) in dioxane (10 mL) and water (2 mL) was stirred at 90° C. for 2 hours under nitrogen. After cooling to room temperature, the reaction was diluted with ethyl acetate (60 mL), washed with water (20 mL) and brine (20 mL), dried over sodium sulfate, filtered, and concentrated. The residue was purified by flash chromatography (silica, petroleum ether/ethyl acetate=2/1 then 1/1) to afford 3-(5-amino-2-chloro-4-fluorophenyl)-N-(4-methoxybenzyl)-N-methyl-1,6-naphthyridin-7-amine (80 mg, 0.189 mmol, 34%) as a brown solid. MS (ESI) m/z 595.2 [M+H]⁺

Step 3. 1-[Bis(dimethylamino)methylene]-11H-1,2,3-triazolo[4,5-b]pyridinium 3-oxid hexafluorophosphate (108 mg, 0.310 mmol) was added to a solution of 3-(5-amino-2-chloro-4-fluorophenyl)-N-(4-methoxybenzyl)-N-methyl-1,6-naphthyridin-7-amine (80 mg, 0.189 mmol), 4-(2-cyanopropan-2-yl)picolinic acid (43 mg, 0.226 mmol) and N,N-diisopropylethylamine (98 mg, 0.758 mmol) in N,N-dimethylformamide (3 mL) at room temperature under nitrogen. After stirring at room temperature for 1 h, the reaction mixture was diluted with ethyl acetate (80 mL), washed with water (20 mL) and brine (20 mL), dried over sodium sulfate, filtered, and concentrated. The residue was purified by flash chromatography (silica, petroleum ether/ethyl acetate=2/1 then 1/1) to afford N-(4-chloro-2-fluoro-5-(7-((4-methoxybenzyl)(methyl)amino)-1,6-naphthyridin-3-yl)phenyl)-4-(2-cyanopropan-2-yl)picolinamide (40 mg, 0.067 mmol, 36%) as a brown solid. MS (ESI) m/z 595.2 [M+H]⁺

Step 4. A mixture of N-(4-chloro-2-fluoro-5-(7-((4-methoxybenzyl)(methyl)amino)-1,6-naphthyridin-3-yl)phenyl)-4-(2-cyanopropan-2-yl)picolinamide (40 mg, 0.067 mmol) in trifluoroacetic acid (2 mL) was stirred at room temperature for 1 hour. The reaction mixture was concentrated and the residue was purified by prep-HPLC (Column: Waters Xbridge 21.2×250 mm C18, 10 μm, Mobile Phase: A: water (10 mmol/L ammonium bicarbonate), B: acetonitrile) to afford N-(4-chloro-2-fluoro-5-(7-(methylamino)-1,6-naphthyridin-3-yl)phenyl)-4-(2-cyanopropan-2-yl)picolinamide (8.9 mg, 0.019 mmol, 28%) as a yellow solid. ¹H NMR (400 MHz, DMSO-d₆) δ=10.62 (s, 1H), 9.00 (d, J=0.4 Hz, 1H), 8.86 (d, J=2.4 Hz, 1H), 8.83 (dd, J=5.2, 0.8 Hz, 1H), 8.33 (d, J=1.6 Hz, 1H), 8.28 (dd, J=2.0, 0.8 Hz, 1H), 8.19 (d, J=8.0 Hz, 1H), 7.90 (dd, J=5.2, 2.0 Hz, 1H), 7.82 (d, J=10.4 Hz, 1H), 7.01 (m, 1H), 6.23 (s, 1H), 2.87 (d, J=4.8 Hz, 3H), 1.77 (s, 6H). MS (ESI) m/z 475.1 [M+H]⁺

### Example 38. Synthesis of 4-(2-Cyanopropan-2-yl)-N-(3-fluoro-4-methyl-5-(7-(methylamino)-1,6-naphthyridin-3-yl)phenyl)picolinamide (Compound 38)

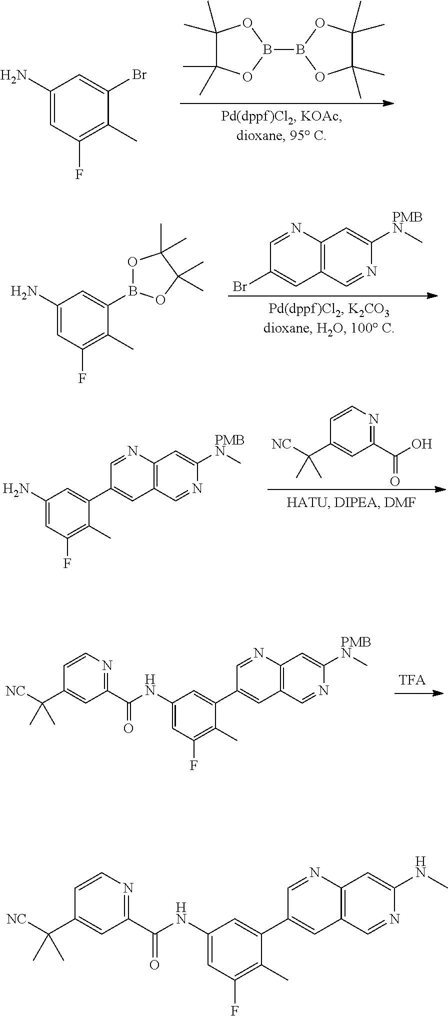

Step 1. A mixture of 3-bromo-5-fluoro-4-methylaniline (500 mg, 2.46 mmol), 4,4,4′,4′,5,5,5′,5′-octamethyl-2,2′-bi(1,3,2-dioxaborolane) (1.23 g, 4.9 mmol), [1,1′-bis(diphenylphosphino)ferrocene]dichloropalladium(II) (201 mg, 0.25 mmol) and potassium acetate (723 mg, 7.38 mmol) in 1,4-dioxane (20 mL) was stirred at 95° C. for 16 hours under nitrogen. After cooling to room temperature, the solvent was removed. The residue was purified by flash chromatography (silica, petroleum ether/ethyl acetate=10/1) to afford 3-fluoro-4-methyl-5-(4,4,5,5-tetramethyl-1,3,2-dioxaborolan-2-yl)aniline (600 mg, 2.39 mmol, 97%) as a yellow solid. MS (ESI) m/z 252.4 [M+H]⁺

Step 2. A mixture of 3-fluoro-4-methyl-5-(4,4,5,5-tetramethyl-1,3,2-dioxaborolan-2-yl)aniline (250 mg, 1 mmol), 3-bromo-N-(4-methoxybenzyl)-N-methyl-1,6-naphthyridin-7-amine (237 mg, 0.66 mmol), [1,1′-bis(diphenylphosphino)ferrocene]dichloropalladium(II) (54 mg, 0.066 mmol) and potassium carbonate (182 mg, 1.32 mmol) in water (2 mL) and 1,4-dioxane (10 mL) was stirred at 100° C. for 4 hours under nitrogen. The reaction mixture was diluted with water (20 mL) and extracted with ethyl acetate (10 mL×3). The combined organic phases were washed brine (10 mL×2), dried over sodium sulfate, filtered and concentrated. The residue was purified by flash chromatography (silica, petroleum ether/ethyl acetate=1/1) to afford 3-(5-amino-3-fluoro-2-methylphenyl)-N-(4-methoxybenzyl)-N-methyl-1,6-naphthyridin-7-amine (230 mg, 0.57 mmol, 57%) as a yellow solid. MS (ESI) m/z 403.2 [M+H]⁺

Step 3. A mixture of 4-(2-cyanopropan-2-yl)picolinic acid (108 mg, 0.57 mmol), 3-(5-amino-3-fluoro-2-methylphenyl)-N-(4-methoxybenzyl)-N-methyl-1,6-naphthyridin-7-amine (230 mg, 0.57 mmol), 1-[bis(dimethylamino)methylene]-1H-1,2,3-triazolo[4,5-b]pyridinium 3-oxid hexafluorophosphate (260 mg, 0.68 mmol) and N,N-diisopropylethylamine (147 mg, 1.14 mmol) in N,N-dimethylformamide (3 mL) was stirred at room temperature for 2 hours. The mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (silica, petroleum ether/ethyl acetate=1/1) to afford 4-(2-cyanopropan-2-yl)-N-(3-fluoro-5-(7-((4-methoxybenzyl)(methyl)amino)-1,6-naphthyridin-3-yl)-4-methylphenyl)picolinamide (110 mg, 0.19 mmol, 34%) as a yellow solid. MS (ESI) m/z 575.2 [M+H]⁺

Step 4. A solution of 4-(2-cyanopropan-2-yl)-N-(3-fluoro-5-(7-((4-methoxybenzyl)(methyl)amino)-1,6-naphthyridin-3-yl)-4-methylphenyl)picolinamide (110 mg, 0.19 mmol) in trifluoroacetic acid (5 mL) was stirred at room temperature for 16 hours. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(3-fluoro-4-methyl-5-(7-(methylamino)-1,6-naphthyridin-3-yl)phenyl)picolinamide (34.9 mg, 0.08 mmol, 40%) as a yellow solid. ¹H NMR (400 MHz, DMSO-d₆) δ 10.97 (s, 1H), 9.00 (s, 1H), 8.84 (d, J=2.4 Hz, 1H), 8.81 (d, J=4.8 Hz, 1H), 8.30-8.28 (m, 2H), 7.95 (dd, J=12.0 Hz, 2.0, 1H), 7.87-7.84 (m, 2H), 6.99 (s, 1H), 6.64 (s, 1H), 2.88 (d, J=4.4 Hz, 3H), 2.20 (d, J=2.0 Hz, 3H), 1.77 (s, 6H). MS (ESI) m/z 455.4 [M+H]⁺

### Example 39. Synthesis of 4-(2-Cyanopropan-2-yl)-N-(3,4-difluoro-5-(7-(methylamino)-1,6-naphthyridin-3-yl)phenyl)picolinamide (Compound 39)

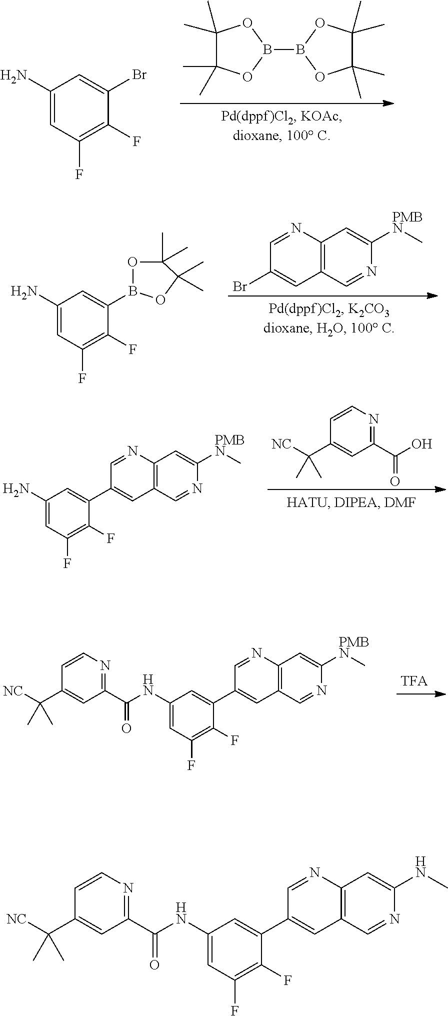

Step 1. A mixture of 3-bromo-4,5-difluoroaniline (414 mg, 2.0 mmol), 4,4,4′,4′,5,5,5′,5′-octamethyl-2,2′-bi(1,3,2-dioxaborolane) (1 g, 4.0 mmol), [1,1′-bis(diphenylphosphino)ferrocene]dichloropalladium(II) (146 mg, 0.2 mmol) and potassium acetate (392 mg, 4.0 mmol) in 1,4-dioxane (5 mL) was stirred at 100° C. for 2 hours under nitrogen.

The reaction mixture was diluted with ethyl acetate (10 mL) and saturated aqueous sodium bicarbonate (10 mL). The organic phase was separated. The aqueous phase was extracted with ethyl acetate (10 mL×2). The combined organic phases were washed with brine (10 mL×2). The solution was dried over anhydrous sodium sulfate, filtered, and concentrated. The crude mixture was purified by flash chromatography (silica, petroleum ether/ethyl acetate=1/1) to afford 3,4-difluoro-5-(4,4,5,5-tetramethyl-1,3,2-dioxaborolan-2-yl)aniline (450 mg, 1.77 mmol, 89%) as a yellow solid. MS (ESI) m/z 256.1 [M+H]⁺

Step 2. A mixture of 3,4-difluoro-5-(4,4,5,5-tetramethyl-1,3,2-dioxaborolan-2-yl)aniline (128 mg, 0.5 mmol), 3-bromo-N-(4-methoxybenzyl)-N-methyl-1,6-naphthyridin-7-amine (214 mg, 0.6 mmol), [1,1′-bis(diphenylphosphino)ferrocene]dichloropalladium(II) (37 mg, 0.05 mmol) and potassium carbonate (138 mg, 1.0 mmol) in water (0.2 mL) and 1,4-dioxane (2 mL) was stirred at 90° C. for 1 hour. The reaction mixture was diluted with water (20 mL) and extracted with ethyl acetate (10 mL×3). The combined organic phases were washed with brine (10 mL×2), dried over sodium sulfate, filtered, and concentrated by rotary evaporation. The crude mixture was purified by flash chromatography (silica, petroleum ether/ethyl acetate=2/3) to afford 3-(5-amino-2,3-difluorophenyl)-N-(4-methoxybenzyl)-N-methyl-1,6-naphthyridin-7-amine (162 mg, 0.4 mmol, 80%) as a yellow solid. MS (ESI) m/z 407.4 [M+H]⁺

Step 3. To a solution of 3-(5-amino-2,3-difluorophenyl)-N-(4-methoxybenzyl)-N-methyl-1,6-naphthyridin-7-amine (162 mg, 0.4 mmol), 4-(2-cyanopropan-2-yl)picolinic acid (80 mg, 0.4 mmol) and 1-[bis(dimethylamino)methylene]-1H-1,2,3-triazolo[4,5-b]pyridinium 3-oxid hexafluorophosphate (198 mg, 0.52 mmol) in N,N-dimethylformamide (3 mL) was added N,N-diisopropylethylamine (0.3 mL). The mixture was stirred at 25° C. for 1 hour. The mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (silica, ethyl acetate) to afford 4-(2-cyanopropan-2-yl)-N-(3,4-difluoro-5-(7-((4-methoxybenzyl(methyl)amino)-1,6-naphthyridin-3-yl)phenyl)picolinamide (210 mg, 0.36 mmol, 91%) as a brown oil. MS (EST) m/z 579.3 [M+H]⁺

Step 4. A solution of 4-(2-cyanopropan-2-yl)-N-(3,4-difluoro-5-(7-((4-methoxybenzyl)(methyl)amino)-1,6-naphthyridin-3-yl)phenyl)picolinamide (145 mg, 0.25 mmol) in trifluoroacetic acid (20 mL) was stirred at 40° C. for 2 hours. The reaction mixture was concentrated and the residue was purified by prep-HPLC (column: Waters Xbridge 25×150 mm, 5 μm, Mobile phase: A: water (0.05% ammonia hydroxide), B: acetonitrile; B %: 30%-57% in 10 min) to afford 4-(2-cyanopropan-2-yl)-N-(3,4-difluoro-5-(7-(methylamino)-1,6-naphthyridin-3-yl)phenyl)picolinamide (59.1 mg, 0.13 mmol, 52%) as a yellow solid. ¹H NMR (400 MHz, DMSO-d₆) δ 11.09 (s, 1H), 9.03 (s, 1H), 9.00 (s, 1H), 8.83 (d, J=5.2 Hz, 1H), 8.48 (s, 1H), 8.31 (d, J=1.4 Hz, 1H), 8.21-8.12 (m, 1H), 8.13-8.06 (m, 1H), 7.88 (dd, J=5.2, 2.0 Hz, 1H), 7.07 (d, J=4.4 Hz, 1H), 6.63 (s, 1H), 2.88 (d, J=4.8 Hz, 3H), 1.77 (s, 6H). MS (ESI) m/z 459.3 [M+H]⁺

### Example 40. Synthesis of 4-(2-Cyanopropan-2-yl)-N-(3-fluoro-4-methyl-5-(7-(methylamino)-1,6-naphthyridin-3-yl)phenyl)picolinamide (Compound 40)

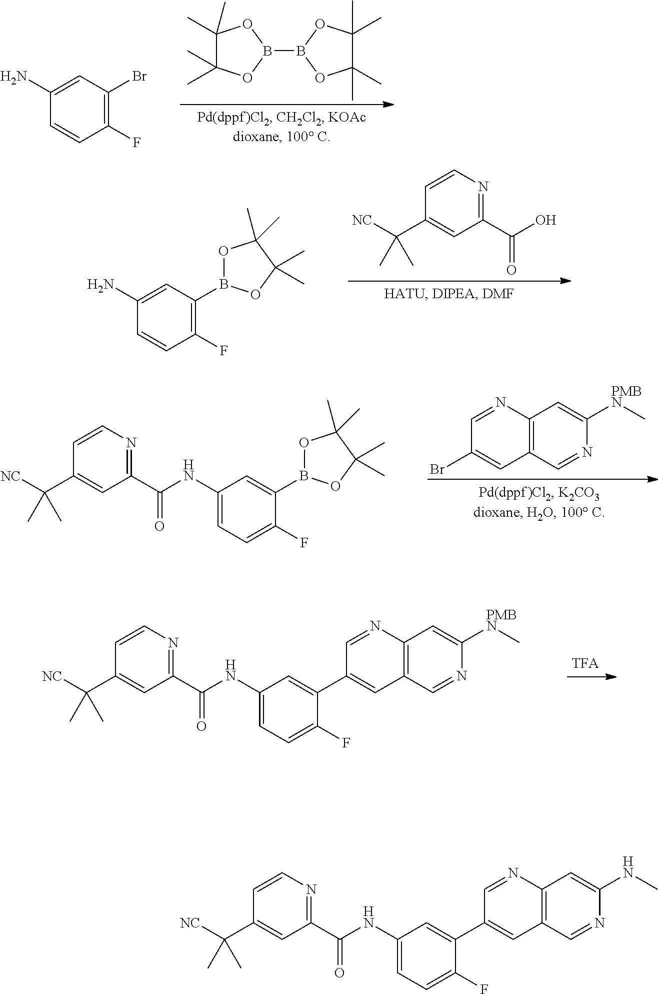

Step 1. A mixture of 3-bromo-4-fluoroaniline (600 mg, 3.16 mmol), 4,4,4′,4′,5,5,5′,5′-octamethyl-2,2′-bi(1,3,2-dioxaborolane) (962 mg, 3.79 mmol), 1,1′-bis(diphenylphosphino)ferrocene-palladium(II) dichloride dichloromethane complex (258 mg, 0.32 mmol) and potassium acetate (619 mg, 6.32 mmol) in 1,4-dioxane (10 mL) was stirred at 100° C. under a nitrogen atmosphere for 2 hours. The mixture was cooled to room temperature and concentrated. The crude residue was purified by flash chromatography (silica, petroleum ether/ethyl acetate=1/1) to afford 4-fluoro-3-(4,4,5,5-tetramethyl-1,3,2-dioxaborolan-2-yl)aniline (330 mg, 1.39 mmol, 44%) as a yellow oil. MS (ESI) m/z 238.1 (M+H)⁺

Step 2. A mixture of 4-(2-cyanopropan-2-yl)picolinic acid (108 mg, 0.57 mmol), 4-fluoro-3-(4,4,5,5-tetramethyl-1,3,2-dioxaborolan-2-yl)aniline (135 mg, 0.57 mmol), 1-[bis(dimethylamino)methylene]-1H-1,2,3-triazolo[4,5-b]pyridinium 3-oxid hexafluorophosphate (260 mg, 0.68 mmol) and N,N-diisopropylethylamine (147 mg, 1.14 mmol) in N,N-dimethylformamide (3 mL) was stirred at room temperature for 2 hours. The mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (silica, petroleum ether/ethyl acetate=1/1) to afford 4-(2-cyanopropan-2-yl)-N-(4-fluoro-3-(4,4,5,5-tetramethyl-1,3,2-dioxaborolan-2-yl)phenyl)picolinamide (180 mg, 0.19 mmol, 78%) as a yellow solid. MS (ESI) m/z 410.1 [M+H]⁺

Step 3. A mixture of 4-(2-cyanopropan-2-yl)-N-(4-fluoro-3-(4,4,5,5-tetramethyl-1,3,2-dioxaborolan-2-yl)phenyl)picolinamide (135 mg, 0.33 mmol), 3-bromo-N-(4-methoxybenzyl)-N-methyl-1,6-naphthyridin-7-amine (118 mg, 0.33 mmol), [1,1′-bis(diphenylphosphino)ferrocene]dichloropalladium(II) (27 mg, 0.033 mmol) and potassium carbonate (91 mg, 0.66 mmol) in water (2 mL) and 1,4-dioxane (10 mL) was stirred at 100° C. for 2 hours under nitrogen. The mixture was cooled to room temperature and concentrated. The reaction mixture was diluted with water (20 mL) and extracted with ethyl acetate (10 mL×3). The combined organic phases were washed brine (10 mL×2), dried over sodium sulfate, filtered and concentrated. The residue was purified by flash chromatography (silica, petroleum ether/ethyl acetate=1/1) to afford 4-(2-cyanopropan-2-yl)-N-(4-fluoro-3-(7-((4-methoxybenzyl)(methyl)amino)-1,6-naphthyridin-3-yl)phenyl)picolinamide (80 mg, 0.14 mmol, 43%) as a yellow solid. MS (ESI) m/z 560.7 [M+H]⁺

Step 4. A solution of 4-(2-cyanopropan-2-yl)-N-(4-fluoro-3-(7-((4-methoxybenzyl)(methyl)amino)-1,6-naphthyridin-3-yl)phenyl)picolinamide (50 mg, 0.09 mmol) in trifluoroacetic acid (5 mL) was stirred at room temperature for 2 hours. The reaction solution was concentrated. The residue was purified by prep-HPLC (Column: Waters Xbridge 21.2×250 mm C18, 10 μm, Mobile Phase: A: water (10 mmol/L ammonium bicarbonate), B: acetonitrile) to afford 4-(2-cyanopropan-2-yl)-N-(4-fluoro-3-(7-(methylamino)-1,6-naphthyridin-3-yl)phenyl)picolinamide (16.2 mg, 0.08 mmol, 41%) as a yellow solid. ¹H NMR (400 MHz, DMSO-d₆) δ 10.93 (s, 1H), 9.01 (d, J=13.8 Hz, 2H), 8.82 (d, J=5.1 Hz, 1H), 8.44 (s, 1H), 8.33-8.20 (m, 2H), 8.02 (s, 1H), 7.86 (dd, J=5.2, 1.9 Hz, 1H), 7.45-7.36 (m, 1H), 7.01 (s, 1H), 6.63 (s, 1H), 2.88 (d, J=4.8 Hz, 3H), 1.75 (d, J=21.7 Hz, 6H). MS (ESI) m/z 441.0 [M+H]⁺

### Example 41. Synthesis of N-(4-Chloro-3-(7-(methylamino)-1,6-naphthyridin-3-yl)phenyl)-4-(2-cyanopropan-2-yl)picolinamide (Compound 41)

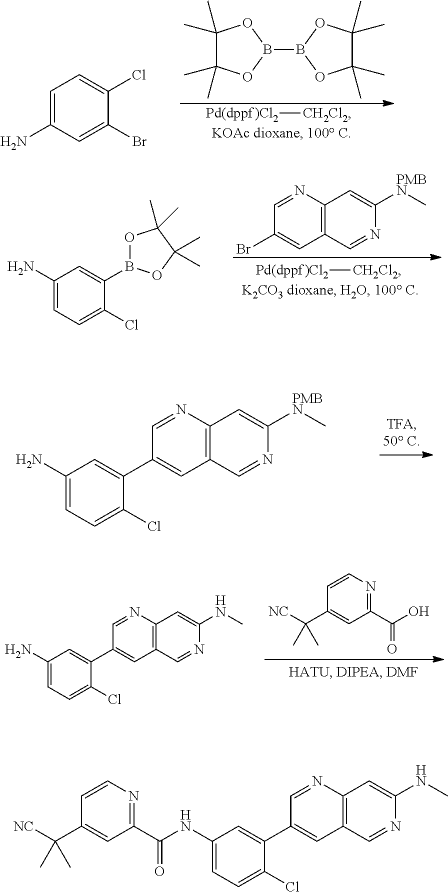

Step 1. A mixture of 3-bromo-4-chloroaniline (618 mg, 3 mmol), 4,4,4′,4′,5,5,5′,5′-octamethyl-2,2′-bi(1,3,2-dioxaborolane) (721 mg, 6 mmol), 1,1′-bis(diphenylphosphino)ferrocene-palladium(II) dichloride dichloromethane complex (245 mg, 0.3 mmol) and potassium acetate (588 mg, 6 mmol) in dioxane (30 mL) was stirred at 100° C. for 2 hours under argon. After cooling to room temperature, the reaction mixture was concentrated. The residue was purified by flash chromatography (silica, petroleum ether/ethyl acetate=4/1) to afford 4-chloro-3-(4,4,5,5-tetramethyl-1,3,2-dioxaborolan-2-yl)aniline (600 mg, 2.37 mmol, 79%) as a yellow oil. MS (ESI) m/z 254.1 [M+H]⁺

Step 2. A solution of 4-chloro-3-(4,4,5,5-tetramethyl-1,3,2-dioxaborolan-2-yl)aniline (167 mg, 0.66 mmol), 3-bromo-N-(4-methoxybenzyl)-N-methyl-1,6-naphthyridin-7-amine (215 mg, 0.6 mmol), 1,1′-bis(diphenylphosphino)ferrocene-palladium(II)dichloride dichloromethane complex (54 mg, 0.066 mmol) and potassium carbonate (166 mg, 1.2 mmol) in dioxane (12 mL) and water (3 ml) was stirred at 100° C. for 2 hours under argon. The reaction mixture was concentrated, extracted with ethyl acetate (50 mL×2), washed with brine (50 mL), dried over sodium sulfate and concentrated. The residue was purified by flash chromatography (silica, petroleum ether/ethyl acetate=3/2) to afford 3-(5-amino-2-chlorophenyl)-N-(4-methoxybenzyl)-N-methyl-1,6-naphthyridin-7-amine (180 mg, 0.45 mmol, 68%) as a yellow oil. MS (ESI) m/z 405.1 [M+H]⁺

Step 3. A solution of 3-(5-amino-2-chlorophenyl)-N-(4-methoxybenzyl)-N-methyl-1,6-naphthyridin-7-amine (180 mg, 0.45 mmol) in trifluoroacetic acid (5 mL) was stirred at 50° C. for 1 hour. Upon completion, the reaction mixture was concentrated, dissolved in methanol (5 mL) and neutralized with sodium bicarbonate. Then the mixture was concentrated and purified by flash chromatography (silica, dichloromethane/methanol=90/1) to afford 3-(5-amino-2-chlorophenyl)-N-methyl-1,6-naphthyridin-7-amine (95 mg, 0.33 mmol, 74%) as a yellow solid. MS (ESI) m/z 284.9 [M+H]⁺

Step 4. A solution of 3-(5-amino-2-chlorophenyl)-N-methyl-1,6-naphthyridin-7-amine (45 mg, 0.16 mmol), 4-(2-cyanopropan-2-yl)picolinic acid (30 mg, 0.16 mmol), 1-[bis(dimethylamino)methylene]-1H-1,2,3-triazolo[4,5-b]pyridinium 3-oxid hexafluorophosphate (91 mg, 0.24 mmol) and N,N-diisopropylethylamine (62 mg, 0.48 mmol) in N,N-dimethylformamide (2 mL) was stirred at 25° C. for 1 hour. The reaction mixture was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford N-(4-chloro-3-(7-(methylamino)-1,6-naphthyridin-3-yl)phenyl)-4-(2-cyanopropan-2-yl)picolinamide (54.4 mg, 0.12 mmol, 75%) as a yellow solid. ¹H NMR (500 MHz, DMSO-d₆) δ 10.99 (s, 1H), 9.01 (s, 1H), 8.89 (d, J=2.3 Hz, 1H), 8.81 (d, J=5.2 Hz, 1H), 8.34 (d, J=2.2 Hz, 1H), 8.28 (d, J=1.9 Hz, 1H), 8.18 (d, J=2.5 Hz, 1H), 8.06 (dd, J=8.8, 2.5 Hz, 1H), 7.86 (dd, J=5.2, 2.0 Hz, 1H), 7.64 (d, J=8.8 Hz, 1H), 6.98 (q, J=4.8 Hz, 1H), 6.64 (s, 1H), 2.88 (d, J=4.9 Hz, 3H), 1.77 (s, 6H). MS (ESI) m/z 456.8 [M+H]⁺

### Example 42. Synthesis of 4-(2-Cyanopropan-2-yl)-N-(2-fluoro-4-methyl-5-(7-(methylamino)-1,6-naphthyridin-3-yl)phenyl)picolinamide (Compound 42)

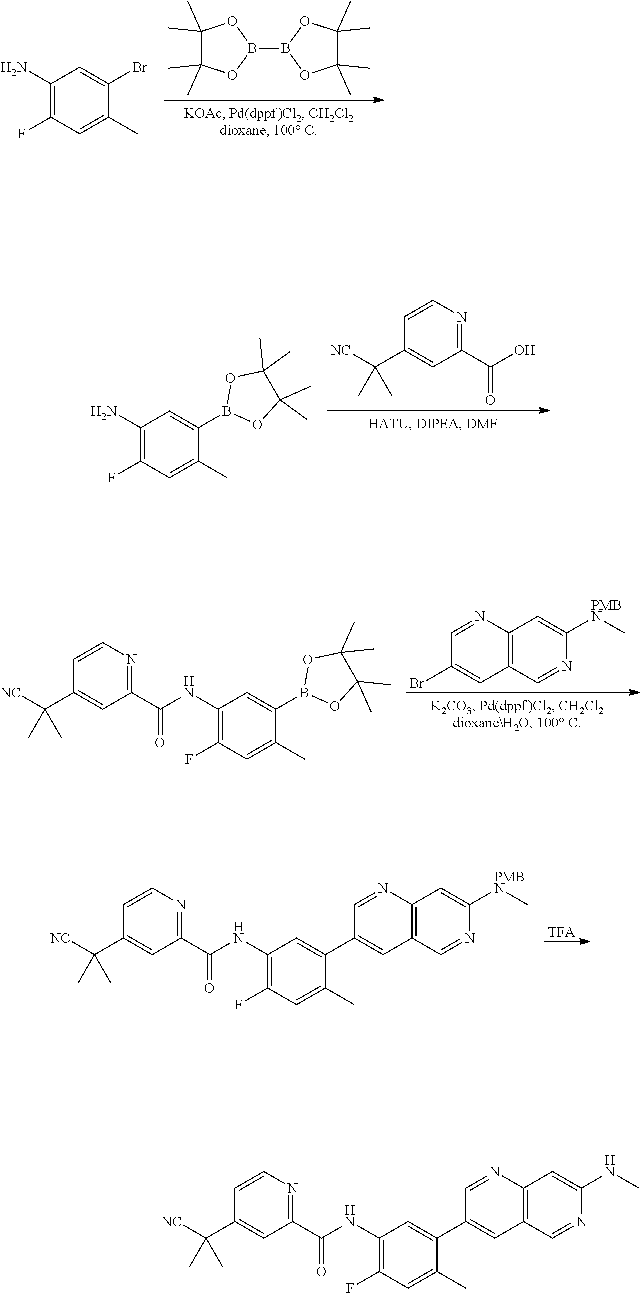

Step 1. A solution of 5-bromo-2-fluoro-4-methylaniline (1.0 g, 4.9 mmol), 4,4,4′,4′,5,5,5′,5′-octamethyl-2,2′-bi(1,3,2-dioxaborolane) (1.87 g, 7.35 mmol), 1,1′-bis(diphenylphosphino)ferrocene-palladium(II) dichloride dichloromethane complex (334 mg, 0.49 mmol) and potassium acetate (960 mg, 9.8 mmol) in 1,4-dioxane (100.0 mL) was stirred at 100° C. under nitrogen atmosphere for 1 hour. The mixture was cooled to room temperature and concentrated. The crude residue was purified by flash chromatography (silica, petroleum ether/ethyl acetate=3/1) to afford 2-fluoro-4-methyl-5-(4,4,5,5-tetramethyl-1,3,2-dioxaborolan-2-yl)aniline (1.2 g, 4.78 mmol, 98%) as a colorless oil. MS (ESI) m/z 252.1[M+H]⁺

Step 2. To a solution of 4-(2-cyanopropan-2-yl)picolinic acid (200 mg, 1.05 mmol) in N,N-dimethylformamide (3 mL) were added 2-fluoro-4-methyl-5-(4,4,5,5-tetramethyl-1,3,2-dioxaborolan-2-yl)aniline (264 mg, 1.05 mmol), 1-[bis(dimethylamino)methylene]-1H-1,2,3-triazolo[4,5-b]pyridinium 3-oxid hexafluorophosphate (439 mg, 1.15 mmol) and N,N-diisopropylethylamine (271 mg, 2.1 mmol) at room temperature. The reaction mixture was stirred room temperature for 2 hours. The reaction mixture was diluted with water. The solid was collected by filtration and dried to afford 4-(2-cyanopropan-2-yl)-N-(2-fluoro-4-methyl-5-(4,4,5,5-tetramethyl-1,3,2-dioxaborolan-2-yl)phenyl)picolinamide (300 mg, 0.71 mmol, 68%) as a white solid. MS (EST) m/z 424.0 [M+H]⁺

Step 3. A mixture of 4-(2-cyanopropan-2-yl)-N-(2-fluoro-4-methyl-5-(4,4,5,5-tetramethyl-1,3,2-dioxaborolan-2-yl)phenyl)picolinamide (300 mg, 0.71 mmol), 3-bromo-N-(4-methoxybenzyl)-N-methyl-1,6-naphthyridin-7-amine (253 mg, 0.71 mmol), 1,1′-bis(diphenylphosphino)ferrocene-palladium(II)dichloride dichloromethane complex (58 mg, 0.071 mmol) and potassium carbonate (196 mg, 1.42 mmol) in 1,4-dioxane/water=5/1 (6 mL) was stirred at 100° C. under nitrogen for 1 hour. The mixture was cooled to room temperature and concentrated. The crude residue was purified flash chromatography (silica, petroleum ether/ethyl acetate=2/3) to afford 4-(2-cyanopropan-2-yl)-N-(2-fluoro-5-(7-((4-methoxybenzyl)(methyl)amino)-1,6-naphthyridin-3-yl)-4-methylphenyl)picolinamide (200 mg, 0.35 mmol, 98%) as a yellow oil. MS (ESI) m/z 575.1 [M+H]⁺

Step 4. A solution of 4-(2-cyanopropan-2-yl)-N-(2-fluoro-5-(7-((4-methoxybenzyl)(methyl)amino)-1,6-naphthyridin-3-yl)-4-methylphenyl)picolinamide (200 mg, 0.35 mmol) in trifluoroacetic acid (4 mL) was stirred at room temperature overnight. The reaction mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-700% in 15 min) to afford 4-(2-cyanopropan-2-yl)-N-(2-fluoro-4-methyl-5-(7-(methylamino)-1,6-naphthyridin-3-yl)phenyl)picolinamide (93.9 mg, 0.21 mmol, 51%) as a yellow solid. ¹H NMR (500 MHz, DMSO-d₆) δ 10.47 (s, 1H), 8.98 (s, 1H), 8.82-8.81 (m, 2H), 8.26 (s, 1H), 8.24 (s, 1H), 7.99 (d, J=8.0 Hz, 1H), 7.88-7.87 (m, 1H), 7.39 (d, J=11.0 Hz, 1H), 6.92 (s, 1H), 6.62 (s, 1H), 2.87 (d, J=4.0 Hz, 3H), 2.31 (s, 3H), 1.76 (s, 6H). MS (EST) m/z 455.1 [M+H]⁺

### Example 43. Synthesis of 4-(2-Cyanopropan-2-yl)-N-(2,4-difluoro-5-(7-(methylamino)-1,6-naphthyridin-3-yl)phenyl)picolinamide (Compound 43)

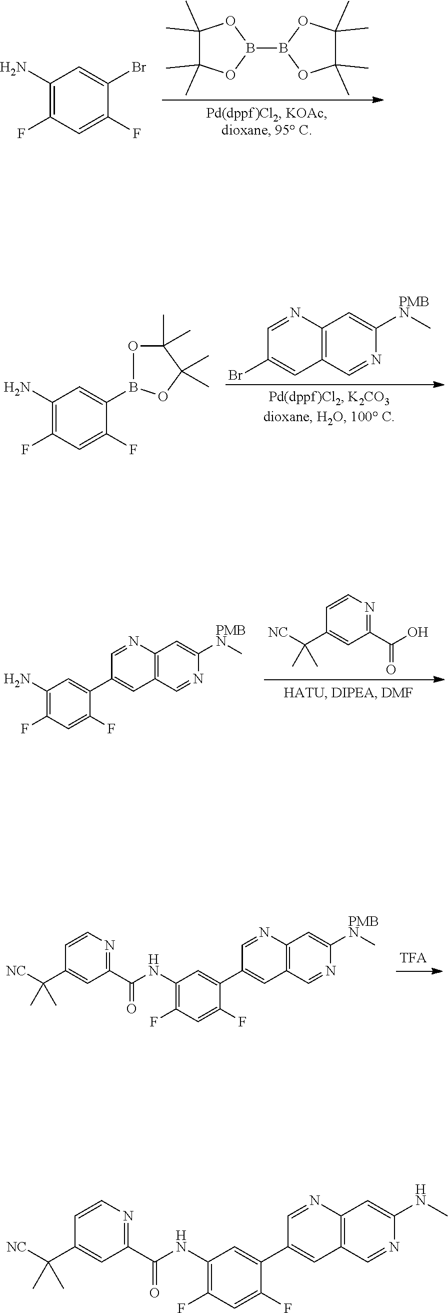

Step 1. A mixture of 5-bromo-2,4-difluoroaniline (300 mg, 1.45 mmol), 4,4,4′,4′,5,5,5′,5′-octamethyl-2,2′-bi(1,3,2-dioxaborolane) (736 mg, 2.89 mmol), [1,1′-bis(diphenylphosphino)ferrocene]dichloropalladium(II) (122 mg, 0.15 mmol) and potassium acetate (426 mg, 4.35 mmol) in 1,4-dioxane (15 mL) was stirred at 95° C. for 16 hours under nitrogen. After cooling to room temperature, the solvent was removed. The residue was purified by flash chromatography (silica, petroleum ether/ethyl acetate=10/1) to afford 2,4-difluoro-5-(4,4,5,5-tetramethyl-1,3,2-dioxaborolan-2-yl)aniline (250 mg, 0.98 mmol, 68%) as a yellow solid. MS (ESI) m/z 256.1 [M+H]⁺

Step 2. A mixture of 2,4-difluoro-5-(4,4,5,5-tetramethyl-1,3,2-dioxaborolan-2-yl)aniline (100 mg, 0.39 mmol), 3-bromo-N-(4-methoxybenzyl)-N-methyl-1,6-naphthyridin-7-amine (93 mg, 0.26 mmol), [1,1′-bis(diphenylphosphino)ferrocene]dichloropalladium(II) (21 mg, 0.026 mmol) and potassium carbonate (72 mg, 0.52 mmol) in water (1 mL) and 1,4-dioxane (5 mL) was stirred at 100° C. for 4 hours under nitrogen. After cooling to room temperature, the reaction mixture was diluted with water (10 mL) and extracted with ethyl acetate (10 mL×3). The combined organic phases were washed brine (10 mL×2), dried over sodium sulfate, filtered, and concentrated. The residue was purified by flash chromatography (silica, petroleum ether/ethyl acetate=1/1) to afford 3-(5-amino-2,4-difluorophenyl)-N-(4-methoxybenzyl)-N-methyl-1,6-naphthyridin-7-amine (75 mg, 0.18 mmol, 71%) as a yellow solid. MS (ESI) m/z 407.4 [M+H]⁺

Step 3. A mixture of 4-(2-cyanopropan-2-yl)picolinic acid (35 mg, 0.18 mmol), 3-(5-amino-2,4-difluorophenyl)-N-(4-methoxybenzyl)-N-methyl-1,6-naphthyridin-7-amine (75 mg, 0.18 mmol), 1-[bis(dimethylamino)methylene]-1H-1,2,3-triazolo[4,5-b]pyridinium 3-oxid hexafluorophosphate (82 mg, 0.22 mmol) and N,N-diisopropylethylamine (46 mg, 0.36 mmol) in N,N-dimethylformamide (3 mL) was stirred at room temperature for 2 hours. The mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (silica, petroleum ether/ethyl acetate=1/1) to afford 4-(2-cyanopropan-2-yl)-N-(2,4-difluoro-5-(7-((4-methoxybenzyl)(methyl)amino)-1,6-naphthyridin-3-yl)phenyl)picolinamide (42 mg, 0.07 mmol, 40%) as a yellow solid. MS (ESI) m/z 579.3 [M+H]⁺

Step 4. A solution of 4-(2-cyanopropan-2-yl)-N-(2,4-difluoro-5-(7-((4-methoxybenzyl)(methyl)amino)-1,6-naphthyridin-3-yl)phenyl)picolinamide (42 mg, 0.07 mmol) in trifluoroacetic acid (2 mL) was stirred at room temperature for 16 hours. The reaction solution was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(2,4-difluoro-5-(7-(methylamino)-1,6-naphthyridin-3-yl)phenyl)picolinamide (15.6 mg, 0.03 mmol, 49%) as a yellow solid. ¹H NMR (400 MHz, DMSO-d₆) δ 10.61 (s, 1H), 9.02 (s, 1H), 8.95 (s, 1H), 8.84 (d, J=5.2 Hz, 1H), 8.82 (s, 1H), 8.29 (s, 1H), 8.18 (t, J=8.4 Hz, 1H), 7.89 (dd, J=5.2 Hz, 2.0 Hz, 1H), 7.64 (t, J=10.4 Hz, 1H), 7.01 (d, J=5.2 Hz, 1H), 6.62 (s, 1H), 2.87 (d, J=4.8 Hz, 3H), 1.77 (s, 6H). MS (ESI) m/z 459.3 [M+H]⁺

### Example 44. Synthesis of N-(4-chloro-3-fluoro-5-(7-(methylamino)-1,6-naphthyridin-3-yl)phenyl)-4-(2-cyanopropan-2-yl)picolinamide (Compound 44)

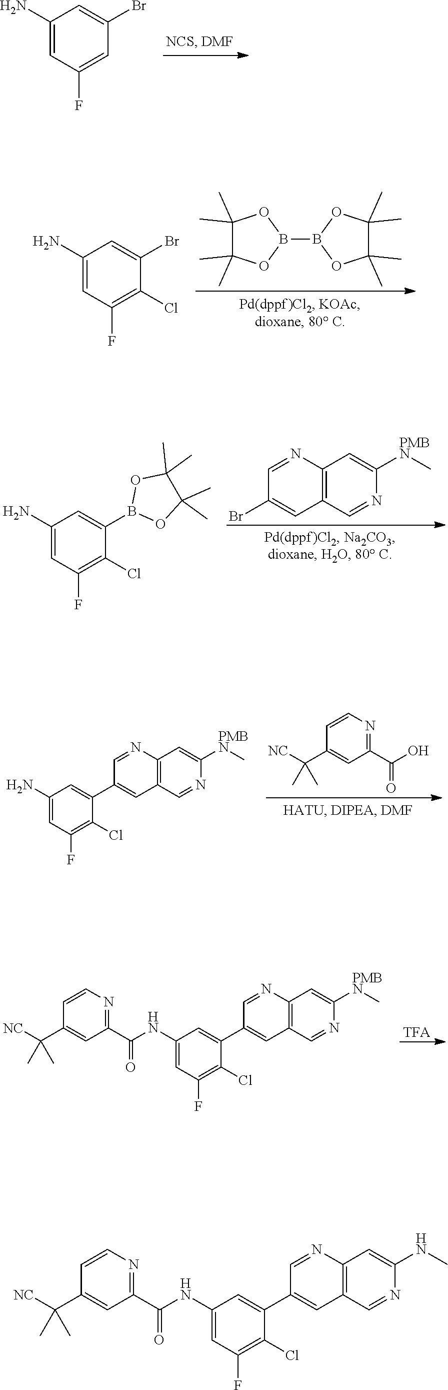

Step 1. A solution of 3-bromo-5-fluoroaniline (900 mg, 5.0 mmol) and N-chlorosuccinimide (731.5 mg, 5.5 mmol) in N,N-dimethylformamide (10 mL) was stirred 60° C. for 6 hours. After cooling to room temperature, the mixture was quenched with water (60 mL) and extracted with ethyl acetate (20 mL×2). The combined organic layers were washed with water (20 mL×3) and brine (10 mL), dried over anhydrous sodium sulfate, filtered and concentrated. The crude residue was purified by flash chromatography (silica, petroleum ether/ethyl acetate=1/1) to afford 3-bromo-4-chloro-5-fluoroaniline (936 mg, 4.0 mmol, 80.8%) as a yellow solid. MS (ESI) m/z 224.9 [M+H]⁺

Step 2. A solution of 3-bromo-4-chloro-5-fluoroaniline (936 mg, 4.0 mmol), 4,4,4′,4′,5,5,5′,5′-octamethyl-2,2′-bi(1,3,2-dioxaborolane) (1.52 g, 6.0 mmol), 1,1′-bis(diphenylphosphino)ferrocene]dichloropalladium(II) (292 mg, 0.4 mmol) and potassium acetate (1.18 g, 12 mmol) in 1,4-dioxane (10 mL) was stirred at 80° C. under argon for 4 hours. After cooling to room temperature, the reaction mixture was quenched with water (50 mL) and extracted with ethyl acetate (100 mL×3). The combined organic layers were dried over sodium sulfate, filtered, concentrated. The residue was purified by flash chromatography (silica, petroleum ether/ethyl acetate=4/1) to afford 4-chloro-3-fluoro-5-(4,4,5,5-tetramethyl-1,3,2-dioxaborolan-2-yl)aniline (545 mg, 2.0 mmol, 50%) as a yellow solid. MS (ESI) m/z 272.1 [M+H]⁺

Step 3. A solution of 4-chloro-3-fluoro-5-(4,4,5,5-tetramethyl-1,3,2-dioxaborolan-2-yl)aniline (545 mg, 2.0 mmol) and 3-bromo-N-(4-methoxybenzyl)-N-methyl-1,6-naphthyridin-7-amine (716 mg, 2.0 mmol) 1,1′-bis(diphenylphosphino)ferrocene]dichloropalladium(II) (140 mg, 0.2 mmol) and sodium carbonate (0.636 g, 6 mmol) in 1,4-dioxane (5 mL) and water (1 mL) was stirred at 80° C. under argon protection for 4 hours. After cooling to room temperature, the reaction mixture was quenched with water (30 mL) and extracted with ethyl acetate (30 mL×3). The combined organic layers were dried over sodium sulfate, filtered and concentrated. The residue was purified by flash chromatography (silica, petroleum ether/ethyl acetate=1/1) to afford 3-(5-amino-2-chloro-3-fluorophenyl)-N-(4-methoxybenzyl)-N-methyl-1,6-naphthyridin-7-amine (422 mg, 1.0 mmol, 500%) as a yellow solid. MS (ESI) m/z 423.1 [M+H]⁺

Step 4. A solution of 4-(2-cyanopropan-2-yl)picolinic acid (38.0 mg, 0.2 mmol), 3-(5-amino-2-chloro-3-fluorophenyl)-N-(4-methoxybenzyl)-N-methyl-1,6-naphthyridin-7-amine (84.4 mg, 0.2 mmol), 1-[bis(dimethylamino)methylene]-1H-1,2,3-triazolo[4,5-b]pyridinium 3-oxid hexafluorophosphate (91.2 mg, 0.24 mmol) and N,N-diisopropylethylamine (77.4 mg, 0.6 mmol) in N,N-dimethyl formamide (1.0 ml) was stirred at 25° C. for 2 hours. The reaction mixture was diluted with ethyl acetate (80 mL), washed with water (20 mL×3) and brine (20 mL), dried over sodium sulfate, filtered and concentrated to afford N-(4-chloro-3-fluoro-5-(7-((4-methoxybenzyl)(methyl)amino)-1,6-naphthyridin-3-yl)phenyl)-4-(2-cyanopropan-2-yl)picolinamide (59.5 mg, 0.1 mmol, 50%) as a yellow solid. MS (ESI) m/z 593.4 [M+H]⁺

Step 5. A solution of N-(4-chloro-3-fluoro-5-(7-((4-methoxybenzyl)(methyl)amino)-1,6-naphthyridin-3-yl)phenyl)-4-(2-cyanopropan-2-yl)picolinamide (59.5 mg, 0.1 mmol) in trifluoroacetic acid (2 mL) was stirred at room temperature for 12 hours and then concentrated. The residue was purified by prep-HPLC (Column. Welch Xtimate 21.2×250 mm C18, 10 μm, Mobile Phase: A: water (10 mmol/L ammonium bicarbonate) B: acetonitrile; B %: 30%-70% in 15 min) to afford N-(4-chloro-3-fluoro-5-(7-(methylamino)-1,6-naphthyridin-3-yl)phenyl)-4-(2-cyanopropan-2-yl)picolinamide (20.0 mg, 0.042 mmol, 42%) as a yellow solid. ¹H NMR (400 MHz, DMSO-d₆) δ 11.18 (s, 1H), 9.02 (s, 1H), 8.91 (s, 1H), 8.82 (s, 1H), 8.29 (s, 1H), 8.16-8.19 (d, 1H), 8.07 (s, 1H), 7.89 (m, 2H), 7.82 (s, 1H), 7.87 (m, 2H), 7.11 (s, 1H), 6.64 (s, 1H), 2.88 (s, 3H), 1.77 (s, 6H). MS (ESI) m/z 475.1 [M+H]⁺

### Example 45. Synthesis of 4-(2-Cyanopropan-2-yl)-N-(2-fluoro-5-(7-(methylamino)-1,6-naphthyridin-3-yl)phenyl)picolinamide (Compound 45)

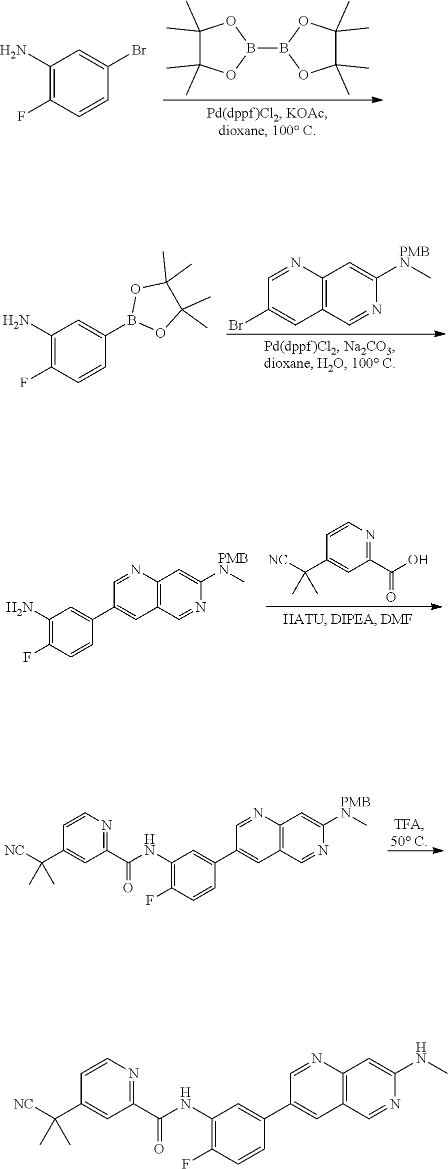

Step 1. A mixture of 5-bromo-2-fluoroaniline (1 g, 5.26 mmol), 4,4,4′,4′,5,5,5′,5′-octamethyl-2,2′-bi(1,3,2-dioxaborolane) (1.6 g, 6.31 mmol), potassium acetate (1.55 g, 15.78 mmol) and [1,1′-bis(diphenylphosphino)ferrocene]dichloropalladium(II) (763 mg, 1.05 mmol) in 1,4-dioxane (10 mL) was stirred at 100° C. for 3 hours under nitrogen. The reaction was poured into water (40 mL) and extracted with ethyl acetate (50 mL×3). The combined organic phases were concentrated and purified by flash chromatography (silica, petroleum ether/ethyl acetate=4/1) to afford 2-fluoro-5-(4,4,5,5-tetramethyl-1,3,2-dioxaborolan-2-yl)aniline (600 mg, 2.53 mmol, 48%) as a yellow solid. MS (ESI) m/z 238.2 [M+H]⁺

Step 2. A solution of 2-fluoro-5-(4,4,5,5-tetramethyl-1,3,2-dioxaborolan-2-yl)aniline (600 mg, 2.53 mmol), 3-bromo-N-(4-methoxybenzyl)-N-methyl-1,6-naphthyridin-7-amine (903 mg, 2.53 mmol), sodium carbonate (802 mg, 7.59 mmol) and [1,1′-bis(diphenylphosphino)ferrocene]dichloropalladium(II) (362 mg, 0.51 mmol) in 1,4-dioxane (5 mL) and water (2 mL) was stirred at 100° C. for 3 hours under nitrogen. The reaction was poured into water (20 mL) and extracted with ethyl acetate (50 mL×3). The combined organic phases were concentrated and purified by flash chromatography (silica, dichloromethanelmethanol=100/1) to afford 3-(3-amino-4-fluorophenyl)-N-(4-methoxybenzyl)-N-methyl-1,6-naphthyridin-7-amine (370 mg, 0.95 mmol, 37%) as a yellow solid. MS (ESI) m/z 389.3 [M+H]⁺

Step 3. 3-(3-Amino-4-fluorophenyl)-N-(4-methoxybenzyl)-N-methyl-1,6-naphthyridin-7-amine (70 mg, 0.18 mmol), 4-(2-cyanopropan-2-yl)picolinic acid (35 mg, 0.18 mmol), 1-[bis(dimethylamino)methylene]-1H-1,2,3-triazolo[4,5-b]pyridinium 3-oxid hexafluorophosphate (137 mg, 0.36 mmol) and N,N-diisopropylethylamine (70 mg, 0.54 mmol) were dissolved in N,N-dimethylformamide (2 mL) at room temperature. After stirring at room temperature for 2 h, the reaction mixture was quenched with water (10 mL) and extracted with dichloromethane (30 mL×3). The combined organic phases were concentrated and purified by flash chromatography (silica, dichloromethane/methanol=10/1) to afford 4-(2-cyanopropan-2-yl)-N-(2-fluoro-5-(7-((4-methoxybenzyl)(methyl)amino)-1,6-naphthyridin-3-yl)phenyl)picolinamide (90 mg, 0.16 mmol, 65%) as a yellow solid. MS (ESI) m/z 561.2 [M+H]⁺

Step 4. A solution of 4-(2-cyanopropan-2-yl)-N-(2-fluoro-5-(7-((4-methoxybenzyl)(methyl)amino)-1,6-naphthyridin-3-yl)phenyl)picolinamide (90 mg, 0.15 mmol) in trifluoroacetic acid (4 mL) was stirred at 50° C. overnight. After cooling to room temperature, the mixture was adjusted to pH=8.0 with saturated aqueous sodium bicarbonate solution. The mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(2-fluoro-5-(7-(methylamino)-1,6-naphthyridin-3-yl)phenyl)picolinamide (37.5 mg, 0.08 mmol, 54%) as a yellow solid. ¹H NMR (400 MHz, DMSO-d) δ 9.12 (d, J=2.4 Hz, 1H), 9.01 (d, J=0.4 Hz, 1H), 8.85-8.83 (m, 1H), 8.49-8.44 (m, 2H), 8.32-8.31 (m, 1H), 7.91-7.89 (m, 1H), 7.71-7.67 (m, 1H), 7.53-7.48 (m, 1H), 6.96-6.92 (m, 1H), 6.61 (s, 1H), 2.87-2.86 (d, J=4.8 Hz, 3H), 1.78 (s, 6H). MS (ESI) m/z 441.3 [M+H]⁺

### Example 46. Synthesis of 4-(2-Cyanopropan-2-yl)-N-(2,3-difluoro-5-(7-(methylamino)-1,6-naphthyridin-3-yl)phenyl)picolinamide (Compound 46)

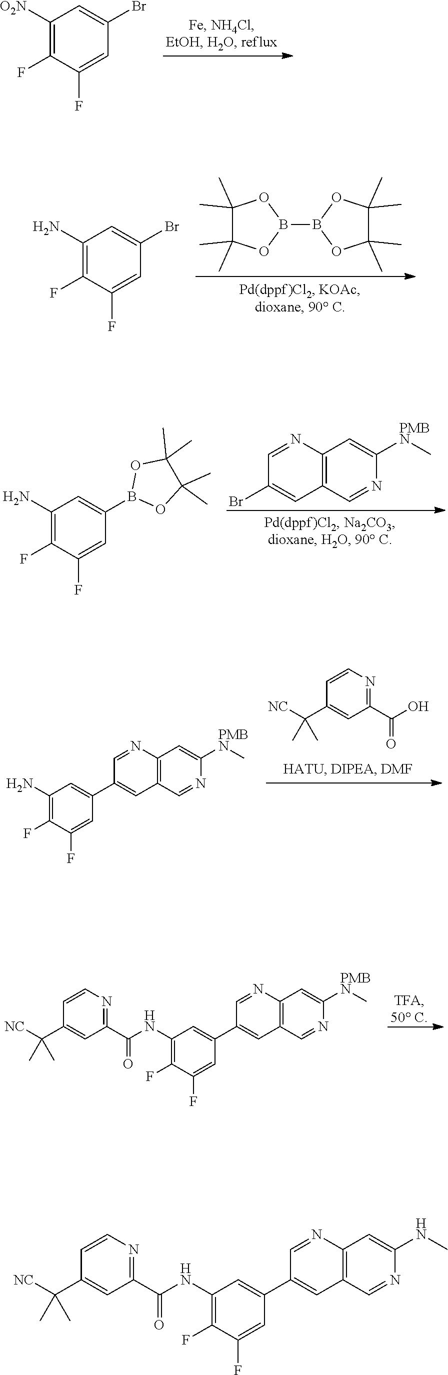

Step 1. A mixture of 5-bromo-1,2-difluoro-3-nitrobenzene (500 mg, 2.11 mmol), iron (236 mg, 4.22 mmol) and ammonium chloride (1.13 g, 21.1 mmol) in ethanol (10 mL) and water (5 mL) was refluxed for 5 hours. The reaction mixture was filtered through celite. The filtrate was concentrated and purified by flash chromatography (silica, petroleum ether/ethyl acetate=1/1) to afford 5-bromo-2,3-difluoroaniline (400 mg, 1.94 mmol, 92%) as a brown oil. MS (ESI) m/z 208.1 [M+H]⁺

Step 2. A solution of 5-bromo-2,3-difluoroaniline (400 mg, 1.94 mmol), 4,4,4′,4′,5,5,5′,5′-octamethyl-2,2′-bi(1,3,2-dioxaborolane) (591 mg, 2.32 mmol), potassium acetate (570 mg, 5.82 mmol) and [1,1′-bis(diphenylphosphino)ferrocene]dichloropalladium(II) (283 mg, 0.39 mmol) in 1,4-dioxane (5 mL) was stirred at 90° C. for 3 hours under nitrogen. After cooling to room temperature, the reaction was poured into water (40 mL) and extracted with ethyl acetate (50 mL×3). The combined organic phases were concentrated and purified by flash chromatography (silica, petroleum ether/ethyl acetate=4/1) to afford 2,3-difluoro-5-(4,4,5,5-tetramethyl-1,3,2-dioxaborolan-2-yl)aniline (250 mg, 0.98 mmol, 50%/o) as a yellow solid. MS (ESI) m/z 256.3 [M+H]⁺

Step 3. A solution of 2,3-difluoro-5-(4,4,5,5-tetramethyl-1,3,2-dioxaborolan-2-yl)aniline (250 mg, 0.98 mmol), 3-bromo-N-(4-methoxybenzyl)-N-methyl-1,6-naphthyridin-7-amine (350 mg, 0.98 mmol), sodium carbonate (311 mg, 2.94 mmol) and [1,1′-bis(diphenylphosphino)ferrocene]dichloropalladium(II) (143 mg, 0.20 mmol) in 1,4-dioxane (5 mL) and water (2 mL) was stirred at 90° C. for 3 hours under nitrogen. After cooling to room temperature, the reaction was poured into water (20 mL) and extracted with ethyl acetate (50 mL×3). The combined organic phases were concentrated and purified by flash chromatography (silica, dichloromethane/methanol=100/1) to afford 3-(3-amino-4,5-difluorophenyl)-N-(4-methoxybenzyl)-N-methyl-1,6-naphthyridin-7-amine (260 mg, 0.64 mmol, 65%) as a yellow solid. MS (EST) m/z 407.1 [M+H]⁺

Step 4. 3-(3-Amino-4,5-difluorophenyl)-N-(4-methoxybenzyl)-N-methyl-1,6-naphthyridin-7-amine (100 mg, 0.24 mmol), 4-(2-cyanopropan-2-yl)picolinic acid (46 mg, 0.24 mmol), I-[bis(dimethylamino)methylene]-1H-1,2,3-triazolo[4,5-b]pyridinium 3-oxid hexafluorophosphate (182 mg, 0.48 mmol) and N,N-diisopropylethylamine (93 mg, 0.72 mmol) were dissolved in N,N-dimethylformamide (2 mL) at room temperature. After stirring at room temperature for 2 h, the reaction mixture was quenched with water (10 mL) and extracted with dichloromethane (30 mL×3). The combined organic phases were concentrated and purified by flash chromatography (silica, dichloromethane/methanol=10/1) to afford 4-(2-cyanopropan-2-yl)-N-(2,3-difluoro-5-(7-((4-methoxybenzyl)(methyl)amino)-1,6-naphthyridin-3-yl)phenyl)picolinamide (90 mg, 0.15 mmol, 65%) as a yellow solid. MS (ESI) m/z 579.3 [M+H]⁺

Step 5. A solution of 4-(2-cyanopropan-2-yl)-N-(2,3-difluoro-5-(7-((4-methoxybenzyl)(methyl)amino)-1,6-naphthyridin-3-yl)phenyl)picolinamide (90 mg, 0.15 mmol) in trifluoroacetic acid (4 mL) was stirred at 50° C. overnight. After cooling to room temperature, the mixture was adjusted to pH=8.0 with saturated aqueous sodium bicarbonate solution. The mixture was concentrated. The residue was purified by prep-HPLC (Column: Welch Xtimate 21.2×250 mm C18, 10 μm, Mobile Phase: A: water (10 mmol/L ammonium bicarbonate) B: acetonitrile; B %: 30%-70% in 15 min) to afford 4-(2-cyanopropan-2-yl)-N-(2,3-difluoro-5-(7-(methylamino)-1,6-naphthyridin-3-yl)phenyl)picolinamide (37.5 mg, 0.08 mmol, 54%) as a yellow solid. ¹H NMR (400 MHz, DMSO-d₆) δ 10.75 (s, 1H), 9.13 (d, J=2.4 Hz, 1H), 8.99 (s, 1H), 8.85 (d, J=5.2 Hz, 1H), 8.54 (d, J=1.6 Hz, 1H), 8.30 (d, J=1.6 Hz, 1H), 8.16 (d, J=6.0 Hz, 1H), 7.91-7.83 (m, 2H), 6.99-6.98 (m, 1H), 6.61 (s, 1H), 2.86 (d, J=4.8 Hz, 3H), 1.78 (s, 6H). MS (ESI) m/z 459.1 [M+H]⁺

### Example 47. Synthesis of 4-(2-Cyanopropan-2-yl)-N-(3-fluoro-5-(7-(methylamino)-1,6-naphthyridin-3-yl)phenyl)picolinamide (Compound 47)

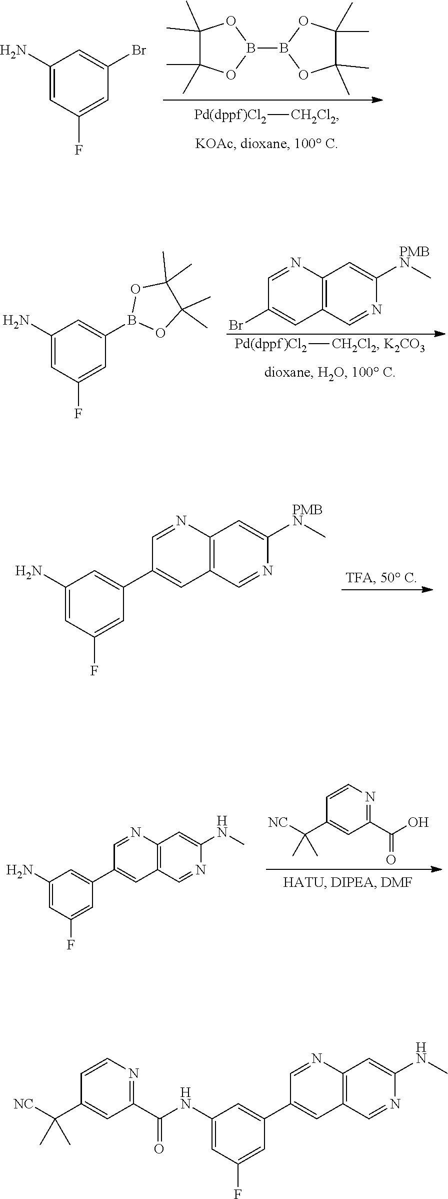

Step 1. A mixture of 3-bromo-5-fluoroaniline (760 mg, 4 mmol), 4,4,4′,4′,5,5,5′,5′-octamethyl-2,2′-bi(1,3,2-dioxaborolane) (1.52 g, 6 mmol), 1,1′-bis(diphenylphosphino)ferrocene-palladium(I) dichloride dichloromethane complex (327 mg, 0.4 mmol) and potassium acetate (784 mg, 8 mmol) in dioxane (35 mL) was stirred at 100° C. for 2 hours under argon. After cooling to room temperature, the reaction mixture was concentrated. The residue was purified by flash chromatography (silica, ethyl acetate/petroleum ether=1/5) to afford 3-fluoro-5-(4,4,5,5-tetramethyl-1,3,2-dioxaborolan-2-yl)aniline (870 mg, 3.67 mmol, 92%) as a yellow oil. MS (ESI) m/z 238.2 [M+H]⁺

Step 2. A mixture of 3-fluoro-5-(4,4,5,5-tetramethyl-1,3,2-dioxaborolan-2-yl)aniline (190 mg, 0.8 mmol), 3-bromo-N-(4-methoxybenzyl)-N-methyl-1,6-naphthyridin-7-amine (260 mg, 0.73 mmol), 1,1′-bis(diphenylphosphino)ferrocene-palladium(II)dichloride dichloromethane complex (65 mg, 0.08 mmol) and potassium carbonate (221 mg, 1.6 mmol) in dioxane (12 mL) and water (3 mL) was stirred at 100° C. for 2 hours under argon. After cooling to room temperature, the reaction mixture was concentrated to remove dioxane. The residue was extracted with ethyl acetate (50 mL×2). The organic layers were washed with brine (50 mL), dried over anhydrous sodium sulfate, filtered and concentrated. The residue was purified by flash chromatography (silica, ethyl acetate/petroleum ether=2/3) to afford 3-(3-amino-5-fluorophenyl)-N-(4-methoxybenzyl)-N-methyl-1,6-naphthyridin-7-amine (145 mg, 0.437 mmol, 47%) as a yellow oil. MS (ESI) m/z 389.1 [M+H]⁺

Step 3. A solution of 3-(3-amino-5-fluorophenyl)-N-(4-methoxybenzyl)-N-methyl-1,6-naphthyridin-7-amine (145 mg, 0.437 mmol) in trifluoroacetic acid (5 mL) was stirred at 50° C. for 1 hour. After cooling to room temperature, the reaction mixture was concentrated to afford 3-(3-amino-5-fluorophenyl)-N-methyl-1,6-naphthyridin-7-amine (99 mg, 0.37 mmol, 1009%) as a brown oil. MS (ESI) m/z 269.3 [M+H]⁺

Step 4. To a solution of 3-(3-amino-5-fluorophenyl)-N-methyl-1,6-naphthyridin-7-amine (32 mg, 0.12 mmol) in N,N-dimethylformamide (2 mL) was added N,N-diisopropylethylamine (77 mg, 0.6 mmol), 4-(2-cyanopropan-2-yl)picolinic acid (27 mg, 0.144 mmol) and 1-[bis(dimethylamino)methylene]-1H-1,2,3-triazolo[4,5-b]pyridinium 3-oxid hexafluorophosphate (68 mg, 0.18 mmol). The mixture was stirred at 25° C. for 1 hour. The mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 4-(2-cyanopropan-2-yl)-N-(3-fluoro-5-(7-(methylamino)-1,6-naphthyridin-3-yl)phenyl)picolinamide (22 mg, 0.05 mmol, 42%) as a yellow solid. ¹H NMR (500 MHz, DMSO-d₆) δ 11.04 (s, 1H), 9.17 (d, J=2.4 Hz, 1H), 9.00 (s, 1H), 8.84 (d, J=5.1 Hz, 1H), 8.56 (d, J=2.2 Hz, 1H), 8.32 (d, 1=1.7 Hz, 1H), 8.26 (s, 1H), 7.97 (d, J=11.1 Hz, 1H), 7.88 (dd, J=5.1, 1.9 Hz, 1H), 7.50 (d, J=10.0 Hz, 1H), 6.98 (q, J=4.8 Hz, 1H), 6.63 (s, 1H), 2.88 (d, J=4.9 Hz, 3H), 1.78 (s, 6H). MS (ESI) m/z 441.1 [M+H]⁺

### Example 48. Synthesis of N-(3-Chloro-4-methyl-5-(7-(methylamino)-1,6-naphthyridin-3-yl)phenyl)-4-(2-cyanopropan-2-yl)picolinamide (Compound 48)

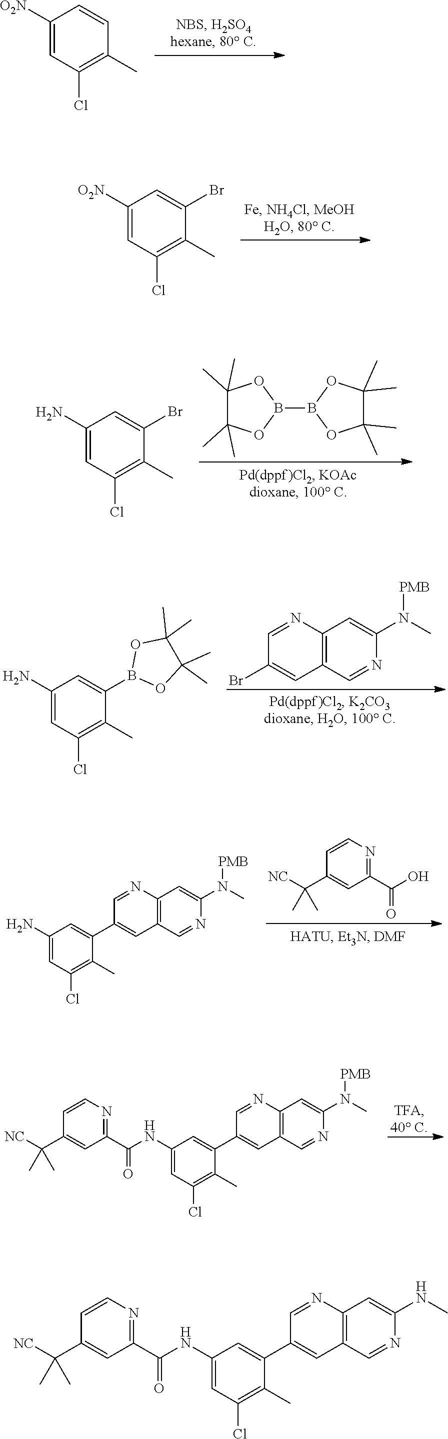

Step 1. To a solution of 2-chloro-1-methyl-4-nitrobenzene (200 mg, 1.17 mmol) in sulfuric acid (3 mL) and hexanes (3 mL) was added N-bromosuccinimide (229 mg, 1.29 mmol) at room temperature. The mixture was stirred at 60° C. for 1 hour. The reaction was cooled to room temperature, treated with water (50 mL) and extracted with ethyl acetate (30 mL×2). The combined organic layers were concentrated. The residue was purified by flash chromatography (silica, petroleum ether/ethyl acetate=5/1) to afford 1-bromo-3-chloro-2-methyl-5-nitrobenzene (150 mg, 0.6 mmol, 51%) as a yellow solid. ¹H NMR (400 MHz, CDCl₃) δ 8.34 (d, J=2.3 Hz, 1H), 8.21 (d, J=2.2 Hz, 1H), 2.61 (s, 3H).

Step 2. The mixture of 1-bromo-3-chloro-2-methyl-5-nitrobenzene (150 mg, 0.6 mmol), iron (169 mg, 3.01 mmol) and ammonium chloride (160 mg, 3.01 mmol) in methanol (3 mL) and water (3 mL) was stirred at 80° C. for 0.5 hour. The reaction was filtered and washed with methanol (3 mL). The filtrate was concentrated. The residue was purified by flash chromatography (silica, petroleum ether/ethyl acetate=2/1) to afford 3-bromo-5-chloro-4-methylaniline (100 mg, 0.46 mmol, 76%) as a yellow oil. MS (ESI) m/z 219.9 [M+H]⁺

Step 3. The mixture of 3-bromo-5-chloro-4-methylaniline (100 mg, 0.46 mmol), 4,4,4′,4′,5,5,5′,5′-octamethyl-2,2′-bi(1,3,2-dioxaborolane) (116 mg, 0.46 mmol), 1,1′-bis(diphenylphosphino)ferrocene-palladium(II)dichloride dichloromethane complex (37 mg, 0.05 mmol) and potassium acetate (89 mg, 0.91 mmol) in dioxane (5 mL) was stirred at 100° C. for 1 hour under nitrogen. The reaction mixture containing 3-chloro-4-methyl-5-(4,4,5,5-tetramethyl-1,3,2-dioxaborolan-2-yl)aniline was used in the next step without further purification. MS (ESI) m/z 268.1 [M+H]⁺

Step 4. The mixture of 3-chloro-4-methyl-5-(4,4,5,5-tetramethyl-1,3,2-dioxaborolan-2-yl)aniline (120 mg, 0.45 mmol), 3-bromo-N-(4-methoxybenzyl)-N-methyl-1,6-naphthyridin-7-amine (160 mg, 0.45 mmol), 1,1′-bis(diphenylphosphino)ferrocene-palladium(II)dichloride dichloromethane complex (37 mg, 0.05 mmol) and potassium carbonate (124 mg, 0.9 mmol) in dioxane (5 mL) and water (2 mL) was stirred at 100° C. for 2 hours under nitrogen. The reaction was cooled to room temperature and concentrated. The residue was purified by flash chromatography (silica, petroleum ether/ethyl acetate=1/2) to afford 3-(5-amino-3-chloro-2-methylphenyl)-N-(4-methoxybenzyl)-N-methyl-1,6-naphthyridin-7-amine (140 mg, 0.33 mmol, 73% over two steps) as a yellow solid. MS (ESI) m/z 419.1 [M+H]⁺

Step 5. The mixture of 3-(5-amino-3-chloro-2-methylphenyl)-N-(4-methoxybenzyl)-N-methyl-1,6-naphthyridin-7-amine (66 mg, 0.16 mmol), 4-(2-cyanopropan-2-yl)picolinic acid (30 mg, 0.16 mmol), 1-[bis(dimethylamino)methylene]-1H-1,2,3-triazolo[4,5-b]pyridinium 3-oxid hexafluorophosphate (72 mg, 0.19 mmol) and triethylamine (48 mg, 0.47 mmol) in N,N-dimethylformamide (2 mL) was stirred at room temperature for 2 hours. The reaction mixture was treated with water (30 mL). The solid was filtered and washed with water (3 mL) to afford N-(3-chloro-5-(7-((4-methoxybenzyl)(methyl)amino)-1,6-naphthyridin-3-yl)-4-methylphenyl)-4-(2-cyanopropan-2-yl)picolinamide (50 mg, 0.085 mmol, 53%) as a yellow solid. MS (ESI) m/z 591.2 [M+H]⁺

Step 6. A solution of N-(3-chloro-5-(7-((4-methoxybenzyl)(methyl)amino)-1,6-naphthyridin-3-yl)-4-methylphenyl)-4-(2-cyanopropan-2-yl)picolinamide (50 mg, 0.085 mmol) in trifluoroacetic acid (1 mL) was stirred at 40° C. for 1 hour. The reaction mixture was cooled to room temperature and concentrated. The residue was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford N-(3-chloro-4-methyl-5-(7-(methylamino)-1,6-naphthyridin-3-yl)phenyl)-4-(2-cyanopropan-2-yl)picolinamide (14.2 mg, 0.03 mmol, 36%) as a yellow solid. ¹H NMR (500 MHz, DMSO-d₆) δ 10.94 (s, 1H), 8.99 (s, 1H), 8.85-8.75 (m, 2H), 8.27 (d, J=1.9 Hz, 2H), 8.22 (d, J=2.1 Hz, 1H), 7.93 (d, J=2.1 Hz, 1H), 7.85 (dd, J=5.2, 1.9 Hz, 1H), 6.99 (s, 1H), 6.63 (s, 1H), 2.88 (d, J=3.8 Hz, 3H), 2.30 (s, 3H), 1.76 (s, 6H). MS (ESI) m/z 470.7 [M+H]⁺

### Example 49. Synthesis of 4-(2-Cyanopropan-2-yl)-N-(6-(7-(methylamino)-1,6-naphthyridin-3-yl)pyridazin-4-yl)picolinamide (Compound 49)

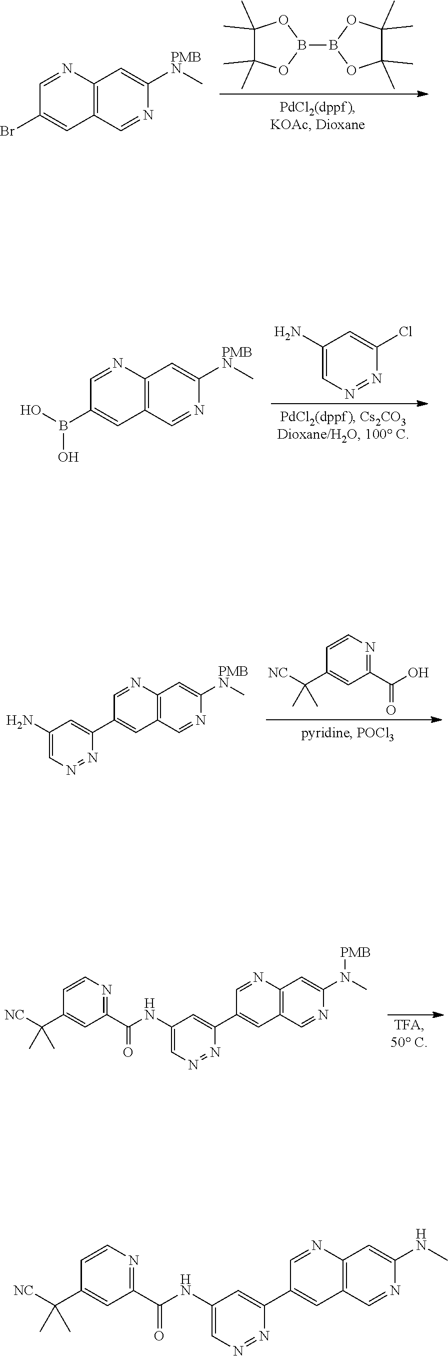

Step 1. A mixture of 3-bromo-N-(4-methoxybenzyl)-N-methyl-1,6-naphthyridin-7-amine (600 mg, 1.68 mmol), 4,4,4′,4′,5,5,5′,5′-octamethyl-2,2′-bi(1,3,2-dioxaborolane) (508 mg, 2.0 mmol), 1,1′-bis(diphenylphosphino)ferrocene-palladium(II)dichloride dichloromethane complex (163 mg, 0.2 mmol) and potassium acetate (600 mg, 6.12 mmol) in dioxane (10 mL) was stirred at 100° C. for 1 hour under nitrogen. After cooling to room temperature, the reaction mixture was used in the next step directly. MS (ESI) m/z 324.1 [M+H]⁺

Step 2. 6-Chloropyridazin-4-amine (260 mg, 2.0 mmol), 1,1′-bis(diphenylphosphino)ferrocene-palladium(II)dichloride dichloromethane complex (163 mg, 0.2 mmol), potassium carbonate (838 mg, 6.0 mmol) and water (5 mL) were added to the reaction solution in the previous step. The resulting mixture was stirred at 100° C. for 2 hours under nitrogen. After cooling to room temperature, the reaction mixture was poured into water (20 mL) and extracted with ethyl acetate (30 mL×3). The combined organic phases were concentrated and purified by flash chromatography (silica, dichloromethane/methanol=10/1) to afford 3-(5-aminopyridazin-3-yl)-N-(4-methoxybenzyl)-N-methyl-1,6-naphthyridin-7-amine (90 mg, 0.24 mmol, 14% over two steps) as a yellow solid. MS (ESI) m/z 373.1 [M+H]⁺

Step 3. To a solution of 3-(5-aminopyridazin-3-yl)-N-(4-methoxybenzyl)-N-methyl-1,6-naphthyridin-7-amine (90 mg, 0.24 mmol) and 4-(2-cyanopropan-2-yl)picolinic acid (45 mg, 0.24 mmol) in pyridine (5 mL) was added phosphoryl trichloride (3 drops) at 0° C. The mixture was stirred at 20° C. for 3 hours. The reaction solution was poured into water (20 mL) and extracted with ethyl acetate (20 mL×3). The combined organic phases were concentrated and purified by flash chromatography (silica, dichloromethane/methanol=10/1) to afford 4-(2-cyanopropan-2-yl)-N-(6-(7-((4-methoxybenzyl)(methyl)amino)-1,6-naphthyridin-3-yl)pyridazin-4-yl)picolinamide (50 mg, 0.09 mmol, 38%) as a yellow solid. MS (ESI) m/z 545.2 [M+H]⁺

Step 4. A solution of 4-(2-cyanopropan-2-yl)-N-(6-(7-((4-methoxybenzyl)(methyl)amino)-1,6-naphthyridin-3-yl)pyridazin-4-yl)picolinamide (50 mg, 0.09 mmol) in trifluoroacetic acid (10 mL) was stirred at 40° C. for 1 hour. After cooling to room temperature, the mixture was concentrated. The residue was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 4-(2-cyanopropan-2-yl)-N-(6-(7-(methylamino)-1,6-naphthyridin-3-yl)pyridazin-4-yl)picolinamide (9.2 mg, 0.02 mmol, 24%) as a yellow solid. ¹H NMR (400 MHz, DMSO-d₆) δ 11.52 (s, 1H), 9.75 (t, J=21.0 Hz, 1H), 9.51 (d, J=2.3 Hz, 1H), 9.12 (s, 1H), 8.97-8.79 (m, 3H), 8.34 (t, J=8.0 Hz, 1H), 7.94 (dd, J=5.2, 2.0 Hz, 1H), 7.14 (d, J=4.8 Hz, 1H), 6.66 (s, 1H), 2.90 (d, J=4.9 Hz, 3H), 1.80 (s, 6H). MS (ESI) m/z 425.1 [M+H]⁺

### Example 50. Synthesis of N-(2-Chloro-4-methyl-5-(7-(methylamino)-1,6-naphthyridin-3-yl)phenyl)-4-(2-cyanopropan-2-yl)picolinamide (Compound 50)

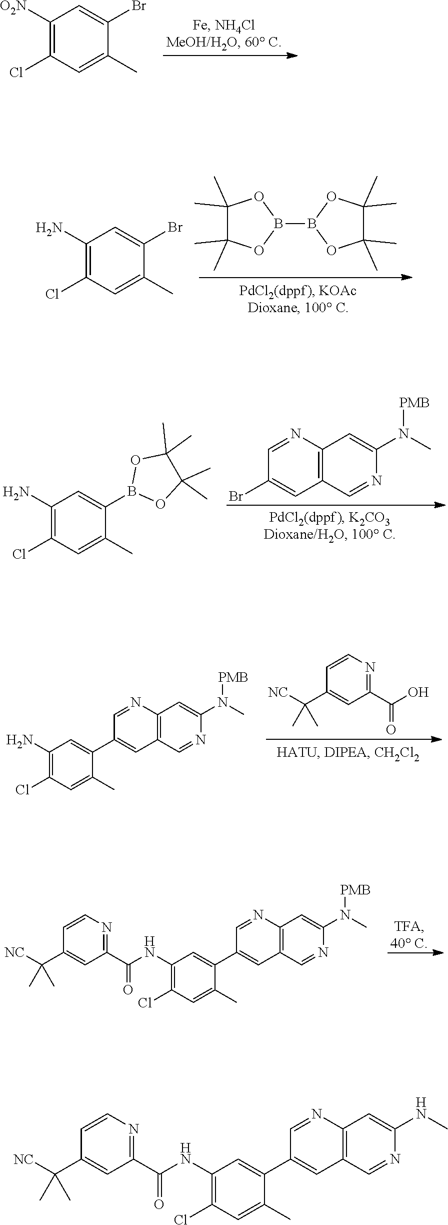

Step 1. A mixture of 1-bromo-4-chloro-2-methyl-5-nitrobenzene (250 mg, 1.0 mmol), iron (224 mg, 4.0 mmol) and ammonium chloride (106 mg, 2.0 mmol) in methanol (6 mL) and water (6 mL) was stirred at 60° C. for 0.5 hour. The reaction was filtered and washed with methanol (30 mL). The filtrate was concentrated. The residue was purified by flash chromatography (silica, petroleum ether/ethyl acetate=5/1) to afford 5-bromo-2-chloro-4-methylaniline (200 mg, 0.91 mmol, 91%) as a yellow oil. MS (ESI) m/z 220.0 [M+H]⁺

Step 2. A mixture of 5-bromo-2-chloro-4-methylaniline (200 mg, 0.91 mmol), 1,1′-bis(diphenylphosphino)ferrocene-palladium(II)dichloride dichloromethane complex (74 mg, 0.09 mmol), 4,4,4′,4′,5,5,5′,5′-octamethyl-2,2′-bi(1,3,2-dioxaborolane) (277 mg, 1.09 mmol) and potassium acetate (267 mg, 2.73 mmol) in dioxane (10 mL) was stirred at 100° C. for 1 hour under nitrogen. The reaction mixture was used in the next step directly. MS (ESI) m/z 268.1 [M+H]⁺

Step 3. 3-Bromo-N-(4-methoxybenzyl)-N-methyl-1,6-naphthyridin-7-amine (270 mg, 0.758 mmol), 1,1′-bis(diphenylphosphino)ferrocene-palladium(II)dichloride dichloromethane complex (62 mg, 0.09 mmol), potassium carbonate (313 mg, 2.274 mmol) and water (5 mL) were added to the reaction solution in the previous step. The resulting mixture was stirred at 100° C. for 2 hours under nitrogen. After cooling to room temperature, the reaction mixture was poured into water (20 mL) and extracted with ethyl acetate (30 mL×3). The combined organic phases were concentrated and purified by flash chromatography (silica, petroleum ether/ethyl acetate=2/1) to afford 3-(5-amino-4-chloro-2-methylphenyl)-N-(4-methoxybenzyl)-N-methyl-1,6-naphthyridin-7-amine (200 mg, 0.478 mmol, 63% over two steps) as a yellow solid. MS (ESI) m/z 419.1 [M+H]⁺

Step 4. A mixture of 3-(5-amino-4-chloro-2-methylphenyl)-N-(4-methoxybenzyl)-N-methyl-1,6-naphthyridin-7-amine (200 mg, 0.478 mmol), 4-(2-cyanopropan-2-yl)picolinic acid (91 mg, 0.478 mmol), 1-[bis(dimethylamino)methylene]-1H-1,2,3-triazolo[4,5-b]pyridinium 3-oxid hexafluorophosphate (272 mg, 0.717 mmol) and N,N-diisopropylethylamine (185 mg, 1.434 mmol) in dichloromethane (12 mL) was stirred at room temperature for 2 hours. The mixture was poured into water (20 mL) and extracted with dichloromethane (20 mL×3). The organic phase was concentrated and purified by flash chromatography (silica, petroleum ether/ethyl acetate=1/2) to afford N-(2-chloro-5-(7-((4-methoxybenzyl)(methyl)amino)-1,6-naphthyridin-3-yl)-4-methylphenyl)-4-(2-cyanopropan-2-yl)picolinamide (250 mg, 0.42 mmol, 89%) as a yellow solid. MS (ESI) m/z 591.2 [M+H]⁺

Step 5. A solution of N-(2-chloro-5-(7-((4-methoxybenzyl)(methyl)amino)-1,6-naphthyridin-3-yl)-4-methylphenyl)-4-(2-cyanopropan-2-yl)picolinamide (250 mg, 0.42 mmol) in trifluoroacetic acid (6 mL) was stirred at 40° C. for 1 hour. The reaction mixture was cooled to room temperature and concentrated. The residue was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford N-(2-chloro-4-methyl-5-(7-(methylamino)-1,6-naphthyridin-3-yl)phenyl)-4-(2-cyanopropan-2-yl)picolinamide (106.2 mg, 0.226 mmol, 54%) as a yellow solid. ¹H NMR (500 MHz, DMSO-d₆) δ 10.62 (s, 1H), 9.00 (s, 1H), 8.88-8.77 (m, 2H), 8.35-8.17 (m, 3H), 7.89 (dd, J=5.2, 2.0 Hz, 1H), 7.63 (s, 1H), 7.12-6.82 (m, 1H), 6.63 (s, 1H), 2.88 (d, J=4.5 Hz, 3H), 2.32 (s, 3H), 1.76 (s, 6H). MS (ESI) m/z 471.1 [M+H]⁺

### Example 51. Synthesis of 4-(2-Cyanopropan-2-yl)-N-(2,3-difluoro-4-methyl-5-(7-(methylamino)-1,6-naphthyridin-3-yl)phenyl)picolinamide (Compound 51)

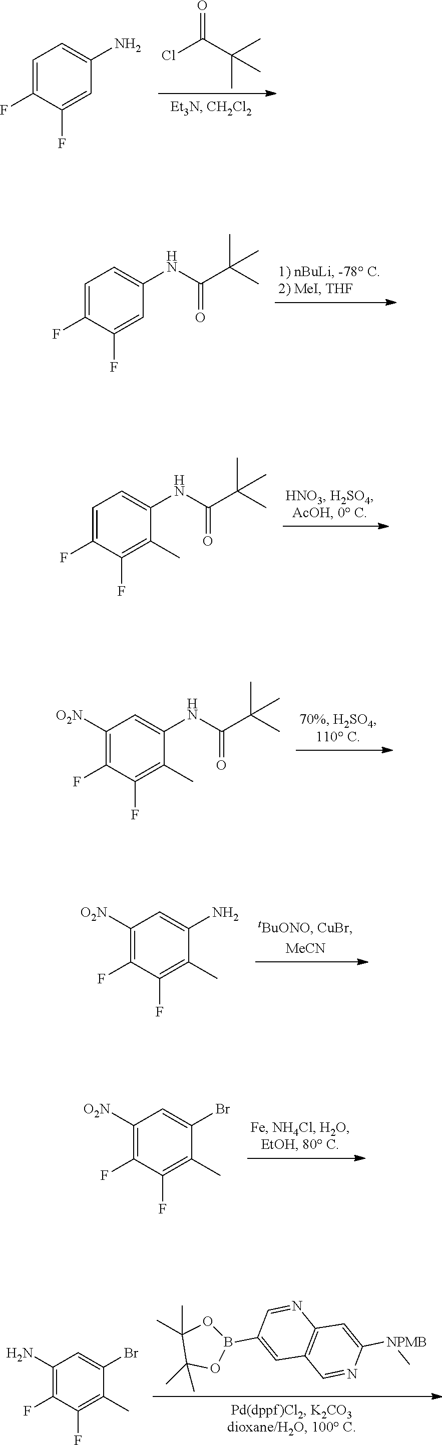

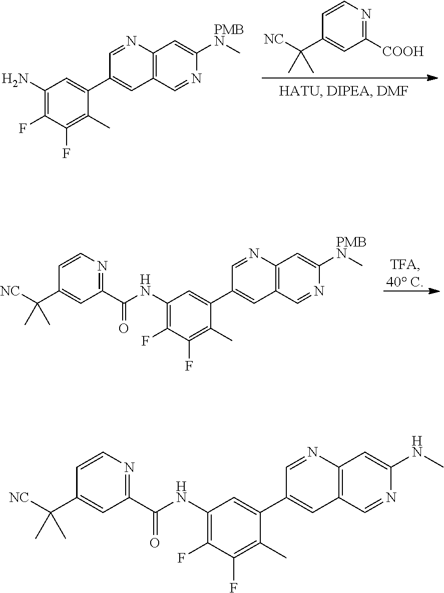

Step 1. To a solution of 3,4-difluoroaniline (1.3 g, 10.0 mmol) in dichloromethane (20 mL) was added pivaloyl chloride (1.36 mL, 11.09 mmol) and triethylamine (1.7 mL, 12.2 mmol) at 0° C. The reaction temperature was allowed to rise to room temperature over 1 hour. The reaction mixture was diluted with dichloromethane (50 mL), washed with water (30 mL) and brine (30 mL). The organic layer was dried over anhydrous sodium sulfate, filtered, and concentrated to afford N-(3,4-difluorophenyl)-2,2-dimethylpropanamide (1.82 g, 8.5 mmol, 85%) as a white solid. MS (ESI) m/z 214.1 [M+H]⁺

Step 2. To a solution of N-(3,4-difluorophenyl)pivalamide (3.2 g, 10.0 mmol) in tetrahydrofuran (60 mL) cooled with dry ice/acetone bath was added n-butyllithium (2.5 M in hexane, 30 mL). After stirring for 1 hour at the same temperature, methyl iodide (1.1 mL, 17 mmol) in tetrahydrofuran (15 mL) was added. The reaction mixture was stirred for 1 hour at the same temperature and then warmed to room temperature and stirred for 2 hours. Then the reaction mixture was quenched with water (100 mL) and extracted with ethyl acetate (100 mL). The organic layer was washed with water (100 mL) and dried over anhydrous sodium sulfate, filtered and concentrated. The residue was purified by flash chromatography (silica, petroleum ether/ethyl acetate=4/1) to afford N-(3,4-difluoro-2-methylphenyl)pivalamide (1.7 g, 7.5 mmol, 50%) as a white solid. MS (ESI) m/z 228.3 [M+H]⁺

Step 3. A solution of N-(3,4-difluoro-2-methylphenyl)pivalamide (570 mg, 2.5 mmol) in acetic acid (0.5 mL) and concentrated sulfuric acid (1.7 mL) was cooled to 0° C. To the solution was added a mixture of fuming nitric acid (0.22 mL) and acetic acid (0.055 mL). After stirring for 1.5 hours at 0° C., the mixture was poured into ice. The mixture was diluted with water (30 mL) and extracted with ethyl acetate (20 mL×3). The organic phases were dried over anhydrous sodium sulfate and concentrated to afford N-(3,4-difluoro-2-methyl-5-nitrophenyl)pivalamide (480 mg, 1.8 mmol, 70%) as a white solid which was used without further purification. MS (ESI) m/z 273.3 [M+H]⁺

Step 4. A mixture of N-(3,4-difluoro-2-methylphenyl)pivalamide (810 mg, 3.0 mmol) in 70% sulfuric acid aqueous solution (30 mL) was stirred at 110° C. overnight. The mixture was cooled to room temperature, diluted with water (100 mL) and extracted with ethyl acetate (50 mL×3). The organic phases were dried over anhydrous sodium sulfate and concentrated to afford 3,4-difluoro-2-methyl-5-nitroaniline (540 mg, 2.87 mmol, 95%) as a white solid which was used without further purification. MS (ESI) m/z 189.3 [M+H]⁺

Step 5. 3,4-Difluoro-2-methyl-5-nitroaniline (540 mg, 2.8 mmol) in acetonitrile (15 mL) was added to a solution of tert-butyl nitrite (590 mg, 5.7 mmol) and cuprous bromide (1.24 mg, 8.6 mmol) in acetonitrile (15 mL) at room temperature. The reaction mixture was stirred at room temperature for 2 hours. The mixture was diluted with water (15 mL) and extracted with ethyl acetate (20 mL×3). The organic phases were dried over anhydrous sodium sulfate and concentrated. The residue was purified by flash chromatography (silica, petroleum ether/ethyl acetate=10/1) to afford 1-bromo-3,4-difluoro-2-methyl-5-nitrobenzene (530 mg, 2.1 mmol, 75%) as a white solid.

Step 6. To a solution of ammonium chloride (910 mg, 16.8 mmol) in ethanol (15 mL) and water (6 mL) was added a mixture of 1-bromo-3,4-difluoro-2-methyl-5-nitrobenzene (530 mg, 2.1 mmol) and iron (950 mg, 16.8 mmol) in ethanol (15 mL). The mixture was stirred at 80° C. for 1 hour. After cooling to room temperature, the reaction mixture was filtered and the filtrate was concentrated to afford 5-bromo-2,3-difluoro-4-methylaniline (430 mg, 1.95 mmol, 93%) as a yellow solid. MS (ESI) m/z 222.7 [M+H]⁺

Step 7. A mixture of 5-bromo-2,3-difluoro-4-methylaniline (44.2 mg, 0.2 mmol), N-(4-methoxybenzyl)-N-methyl-3-(4,4,5,5-tetramethyl-1,3,2-dioxaborolan-2-yl)-1,6-naphthyridin-7-amine (90 mg, 0.22 mmol), [1,1′-bis(diphenylphosphino)ferrocene]dichloropalladium(II) (15 mg, 0.02 mmol) and potassium carbonate (55 mg, 0.4 mmol) in 1,4-dioxane (2 mL) and water (0.2 mL) was stirred at 100° C. for 1 hour under nitrogen. After cooling to room temperature, the reaction mixture was diluted with water (10 mL) and extracted with ethyl acetate (10 mL×3). The organic phases were dried over anhydrous sodium sulfate and concentrated. The residue was purified by flash chromatography (silica, petroleum ether/ethyl acetate=1/9) to afford 3-(5-amino-3,4-difluoro-2-methylphenyl)-N-(4-methoxybenzyl)-N-methyl-1,6-naphthyridin-7-amine (80 mg, 0.19 mmol, 95%) as a yellow solid. MS (ESI) m/z 421.3 [M+H]⁺

Step 8. To an N,N-dimethylformamide (2 mL) solution of 3-(5-amino-2,3-difluorophenyl)-N-(4-methoxybenzyl)-N-methyl-1,6-naphthyridin-7-amine (80 mg, 0.19 mmol), 4-(2-cyanopropan-2-yl)picolinic acid (40 mg, 0.21 mmol) and 1-[bis(dimethylamino)methylene]-1H-1,2,3-triazolo[4,5-b]pyridinium 3-oxid hexafluorophosphate (94 mg, 0.25 mmol) was added N,N-diisopropylethylamine (0.15 mL) at room temperature. After stirring at room temperature for 1 h, the mixture was diluted with water (10 mL) and extracted with ethyl acetate (10 mL×3). The combined organic layers were washed with brine (10 mL), dried over anhydrous sodium sulfate, filtered and concentrated. The residue was purified by flash chromatography (silica, petroleum ether/ethyl acetate=1/9) to afford 4-(2-cyanopropan-2-yl)-N-(2,3-difluoro-5-(7-((4-methoxybenzyl)(methyl)amino)-1,6-naphthyridin-3-yl)-4-methylphenyl)picolinamide (95 mg, 0.16 mmol, 84%). MS (ESI) m/z 593.3 [M+H]⁺

Step 9. A solution of 4-(2-cyanopropan-2-yl)-N-(2,3-difluoro-5-(7-((4-methoxybenzyl)(methyl)amino)-1,6-naphthyridin-3-yl)-4-methylphenyl)picolinamide (50 mg, 0.084 mmol) in trifluoroacetic acid (10 mL) was stirred at 40° C. for 2 hours. The reaction mixture was cooled to room temperature and concentrated. The residue was purified by prep-HPLC (column: Waters Xbridge 25×150 mm, 5 μm, Mobile phase: A: water (0.05% ammonia hydroxide), B: acetonitrile; B %: 30%-57% in 10 min) to afford 4-(2-cyanopropan-2-yl)-N-(2,3-difluoro-4-methyl-5-(7-(methylamino)-1,6-naphthyridin-3-yl)phenyl)picolinamide (21.0 mg, 0.44 mmol, 53%) as a yellow solid. ¹H NMR (400 MHz, DMSO-d₆) δ 10.13 (s, 1H), 8.91 (s, 1H), 8.65 (d, J=2.3 Hz, 1H), 8.34 (d, J=5.2 Hz, 1H), 8.22 (d, J=1.8 Hz, 1H), 8.13 (d, J=1.5 Hz, 1H), 8.03 (dd, J=12.2, 7.9 Hz, 1H), 7.69 (dd, J=5.2, 2.0 Hz, 1H), 6.97 (d, J=5.0 Hz, 1H), 6.61 (s, 1H), 2.86 (d, J=4.9 Hz, 3H), 2.09 (d, J=2.3 Hz, 3H), 1.68 (s, 6H). MS (ESI) m/z 473.4 [M+H]⁺

### Example 52. Synthesis of 3-Fluoro-N-(6-methyl-5-(7-(methylamino)-1,6-naphthyridin-3-yl)pyridin-3-yl)-4-(trifluoromethyl)picolinamide (Compound 52)

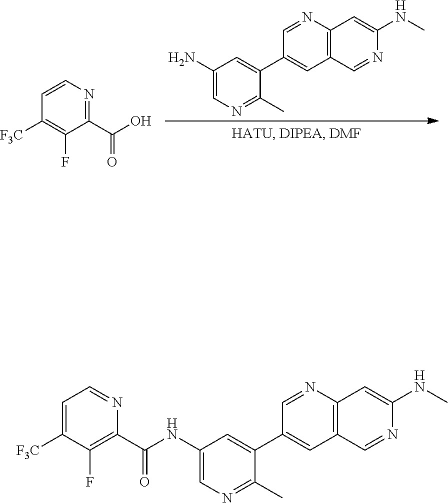 [SMILES: O=C(O)c1nccc(C(F)(F)F)c1F ; CNc1cc2ncc(-c3cc(N)cnc3C)cc2cn1 ; CNc1cc2ncc(-c3cc(NC(=O)c4nccc(C(F)(F)F)c4F)cnc3C)cc2cn1]

A solution of 3-(5-amino-2-methylpyridin-3-yl)-N-methyl-1,6-naphthyridin-7-amine (32 mg, 0.12 mmol), 3-fluoro-4-(trifluoromethyl)picolinic acid (30 mg, 0.14 mmol), 1-[bis(dimethylamino)methylene]-1H-1,2,3-triazolo[4,5-b]pyridinium 3-oxid hexafluorophosphate (80 mg, 0.21 mmol) and N,N-diisopropylethylamine (54 mg, 0.42 mmol) in N,N-dimethylformamide (2 mL) was stirred at 25° C. for 1 hour. The mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 3-fluoro-N-(6-methyl-5-(7-(methylamino)-1,6-naphthyridin-3-yl)pyridin-3-yl)-4-(trifluoromethyl)picolinamide (13.7 mg, 0.03 mmol, 22%) as a yellow solid. ¹H NMR (500 MHz, DMSO-d₆) δ 11.07 (s, 1H), 8.99 (s, 1H), 8.92 (d, J=2.4 Hz, 1H), 8.87 (d, J=2.2 Hz, 1H), 8.83 (d, J=4.8 Hz, 1H), 8.33 (d, J=2.0 Hz, 1H), 8.22 (d, J=2.3 Hz, 1H), 8.15 (t, J=4.9 Hz, 1H), 6.95 (q, J=4.8 Hz, 1H), 6.63 (s, 1H), 2.88 (d, J=4.9 Hz, 3H), 2.50 (s, 3H). MS (ESI) m/z 457.0 [M+H]⁺

### Example 53. Synthesis of N-(4-(2-cyanopropan-2-yl)pyridin-2-yl)-4-methyl-3-(7-(methylamino)-1,6-naphthyridin-3-yl)benzamide (Compound 53)

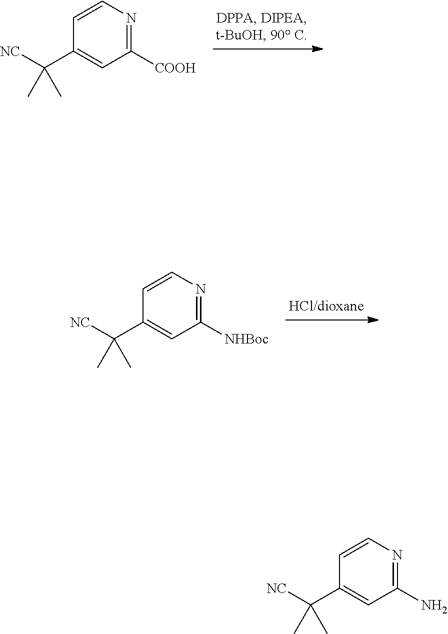 [SMILES: CC(C)(C#N)c1ccnc(C(=O)O)c1 ; CC(C)(C)OC(=O)Nc1cc(C(C)(C)C#N)ccn1 ; CC(C)(C#N)c1ccnc(N)c1]

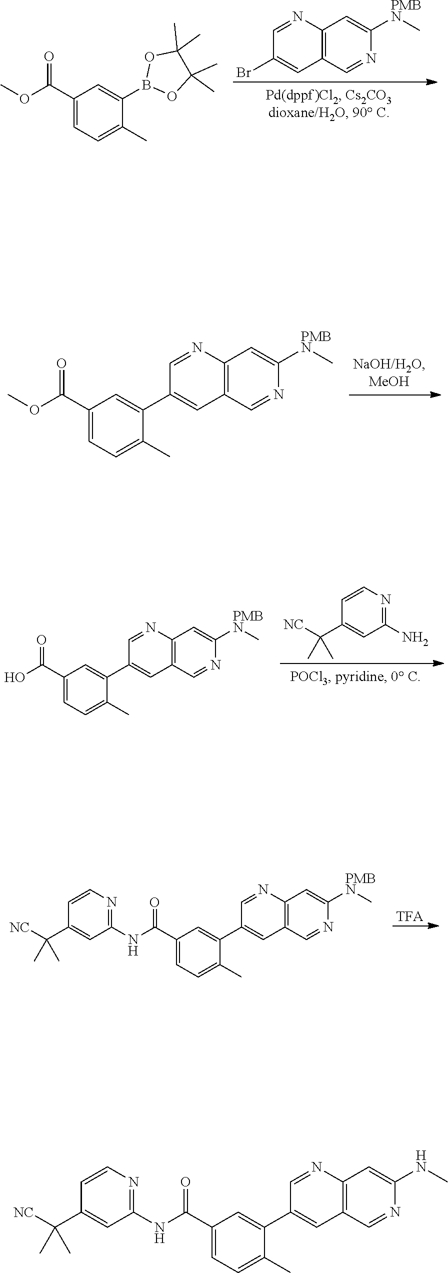

Step 1. 4-(2-Cyanopropan-2-yl)picolinic acid (190 mg, 1 mmol), diphenylphosphoryl azide (302 mg, 1.1 mmol) and N,N-diisopropylethylamine (258 mg, 2 mmol) were dissolved in tert-butanol (4 mL) and stirred at 90° C. for 16 hours. After cooling to room temperature, the mixture was concentrated in vacuo. The residue was diluted with water (30 mL) and extracted with ethyl acetate (3×30 mL). The combined organic phases were concentrated in vacuo. The residue was purified by flash chromatography (silica, ethyl acetate/petroleum ether=0% to 33%) to afford tert-butyl 4-(2-cyanopropan-2-yl)pyridin-2-ylcarbamate (150 mg, 0.57 mmol, 52%) as a white solid. MS (ESI) m/z 262.2 [M+H]⁺

Step 2. tert-Butyl 4-(2-cyanopropan-2-yl)pyridin-2-ylcarbamate (150 mg, 0.57 mmol) was dissolved in 4 M hydrochloride in dioxane (4 mL) and stirred at room temperature for 4 hours. The mixture was concentrated in vacuo to afford 2-(2-aminopyridin-4-yl)-2-methylpropanenitrile (100 mg, 0.62 mmol) as a yellow oil which was used without further purification. MS (ESI) m/z 162.1 [M+H]⁺

Step 3. [1,1′-Bis(diphenylphosphino)ferrocene]dichloropalladium(II) (21 mg, 0.029 mmol) was added to a solution of methyl 4-methyl-3-(4,4,5,5-tetramethyl-1,3,2-dioxaborolan-2-yl)benzoate (200 mg, 0.72 mmol), 3-bromo-N-(4-methoxybenzyl)-N-methyl-1,6-naphthyridin-7-amine (259 mg, 0.72 mmol) and potassium carbonate (468 mg, 1.44 mmol) in dioxane (10 mL) and water (2 mL) at room temperature. The reaction solution was stirred at 90° C. for 2 hours under nitrogen. After cooling to room temperature, the reaction was diluted with ethyl acetate (60 mL), washed with water (20 mL) and brine (20 mL), dried over sodium sulfate, filtered, and concentrated. The residue was purified by flash chromatography (silica, petroleum ether/ethyl acetated=2/1 then 1/1) to afford methyl 3-(7-((4-methoxybenzyl)(methyl)amino)-1,6-naphthyridin-3-yl)-4-methylbenzoate (180 mg, 0.42 mmol, 58% over two steps) as a yellow solid. MS (ESI) m/z 428.1 [M+H]⁺

Step 4. A solution of methyl 3-(7-((4-methoxybenzyl)(methyl)amino)-1,6-naphthyridin-3-yl)-4-methylbenzoate (180 mg, 0.42 mmol) and sodium hydroxide (84 mg, 2.11 mmol) in water (5 mL) and methanol (5 mL) was stirred at room temperature for 3 hours under argon. The reaction mixture was adjusted to pH=3.0 with 2 N hydrogen chloride and concentrated. The residue was diluted with ethyl acetate (25 mL), washed with water (25 mL) and brine (25 mL), dried over sodium sulfate, filtered, and concentrated. The residue was purified by flash chromatography (silica, dichloromethane/methanol=20/1) to afford 3-(7-((4-methoxybenzyl)(methyl)amino)-1,6-naphthyridin-3-yl)-4-methylbenzoic acid (120 mg, 0.29 mmol, 84%) as a yellow solid. MS (ESI) m/z 414.1 [M+H]⁺

Step 5. To a solution of 3-(7-((4-methoxybenzyl)(methyl)amino)-1,6-naphthyridin-3-yl)-4-methylbenzoic acid (30 mg, 0.073 mmol) in pyridine (3 mL) were added 2-(2-aminopyridin-4-yl)-2-methylpropanenitrile (12 mg, 0.073 mmol) and phosphoryl trichloride (97 mg, 0.63 mmol) at 0° C. The reaction mixture was stirred at 0° C. for 1 hour. The reaction mixture was poured to water at 0° C. and extracted with ethyl acetate (30 mL). The organic layer was washed with water (20 mL×3) and brine (20 mL), dried over sodium sulfate, filtered and concentrated. The residue was purified by flash chromatography (silica, ethyl acetate) to afford N-(4-(2-cyanopropan-2-yl)pyridin-2-yl)-3-(7-((4-methoxybenzyl)(methyl)amino)-1,6-naphthyridin-3-yl)-4-methylbenzamide (20 mg, 0.036 mmol, 49%) as a yellow solid. MS (ESI) m/z 557.3 [M+H]⁺

Step 6. A solution of N-(4-(2-cyanopropan-2-yl)pyridin-2-yl)-3-(7-((4-methoxybenzyl)(methyl)amino)-1,6-naphthyridin-3-yl)-4-methylbenzamide (20 mg, 0.036 mmol) in trifluoroacetic acid (2 mL) was stirred at room temperature for 16 hours. Then the solvent was removed. The residue was purified prep-HPLC (Column: Waters Xbridge 21.2×250 mm C18, 10 μm, Mobile Phase: A: water (10 mmol/L ammonium bicarbonate), B: acetonitrile) to afford N-(4-(2-cyanopropan-2-yl)pyridin-2-yl)-4-methyl-3-(7-(methylamino)-1,6-naphthyridin-3-yl)benzamide (2.9 mg, 0.007 mmol, 18%) as a yellow solid. ¹H NMR (400 MHz, DMSO-d₆) δ 11.06 (s, 1H), 8.98 (s, 1H), 8.94 (d, J=2.3 Hz, 1H), 8.48 (s, 1H), 8.44 (d, J=5.3 Hz, 1H), 8.34 (s, 1H), 8.09 (s, 1H), 7.98 (d, J=7.8 Hz, 1H), 7.52 (d, J=8.0 Hz, 1H), 7.32 (dd, J₁=5.3 Hz, J₂=1.7 Hz 1H), 6.91 (d, J₁=4.8 Hz, 1H), 6.64 (s, 1H), 2.88 (d, J=4.9 Hz, 3H), 2.41 (s, 3H) 1.72 (s, 6H). MS (ESI) m/z 436.8 [M+H]⁺

### Example 54. Synthesis of 3-Fluoro-N-(4-methyl-3-(7-(methylamino)-1,6-naphthyridin-3-yl)phenyl)-4-(trifluoromethyl)picolinamide (Compound 54)

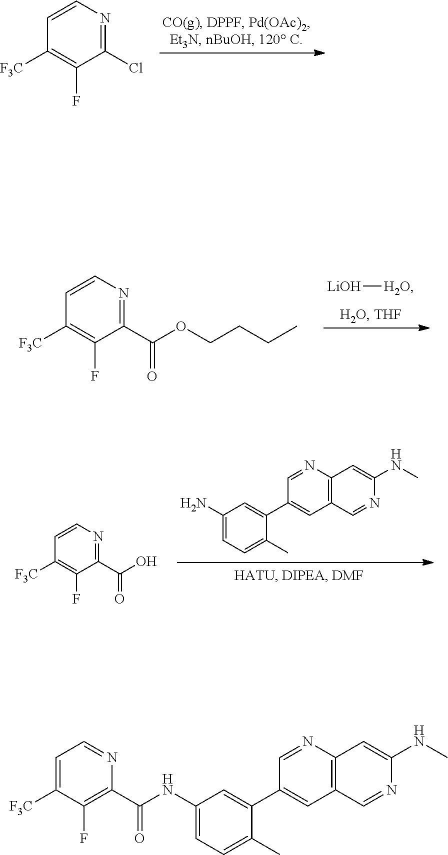

Step 1. A mixture of 2-chloro-3-fluoro-4-(trifluoromethyl)pyridine (1 g, 5 mmol), 1,1′-bis(diphenylphosphino)ferrocene (1.11 g, 2 mmol), palladium (II) acetate (224 mg, 1 mmol) and triethylamine (2.02 g, 20 mmol) in butan-1-ol (250 mL) was stirred at 120° C. for 36 hours under carbon monoxide. The mixture was filtered and washed with butan-1-ol (50 mL×2). The filtrate was concentrated under reduced pressure to afford butyl 3-fluoro-4-(trifluoromethyl)picolinate (1.3 g, crude) as a brown oil. MS (ESI) m/z 266.1 [M+H]⁺

Step 2. To a solution of butyl 3-fluoro-4-(trifluoromethyl)picolinate (1.3 g, 4.9 mmol) in tetrahydrofuran (15 mL) was added lithium hydroxide hydrate (1.03 g, 24.5 mmol) and water (15 mL) at 25° C. After stirring at 25° C. for 1 h, the solvent was evaporated under reduced pressure. The residue was diluted with water (5 mL). The mixture was adjusted to pH=3 with 1 N hydrochloric acid slowly at 0° C. and extracted with ethyl acetate (50 mL×4). The combined organic layers were washed with water (50 mL), dried over sodium sulfate, filtered and concentrated to afford 3-fluoro-4-(trifluoromethyl)picolinic acid (700 mg, 3.35 mmol, 68%) as a white solid. MS (ESI) m/z 210.1 [M+H]⁺

Step 3. A solution of 3-(5-amino-2-methylphenyl)-N-methyl-1,6-naphthyridin-7-amine (48 mg, 0.18 mmol), 3-fluoro-4-(trifluoromethyl)picolinic acid (42 mg, 0.2 mmol), 1-[bis(dimethylamino)methylene]-1H-1,2,3-triazolo[4,5-b]pyridinium 3-oxid hexafluorophosphate (114 mg, 0.3 mmol) and N,N-diisopropylethylamine (77 mg, 0.6 mmol) in N,N-dimethylformamide (2 mL) was stirred at 25° C. for 1 hour. The mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 3-fluoro-N-(4-methyl-3-(7-(methylamino)-1,6-naphthyridin-3-yl)phenyl)-4-(trifluoromethyl)picolinamide (47.1 mg, 0.104 mmol, 52%) as an orange solid. ¹H NMR (500 MHz, DMSO-d₆) δ 10.82 (s, 1H), 9.01 (s, 1H), 8.84 (d, J=2.3 Hz, 1H), 8.81 (d, J=4.8 Hz, 1H), 8.29 (s, 1H), 8.12 (dd, J=10.4, 5.5 Hz, 1H), 7.82 (d, J=2.2 Hz, 1H), 7.76 (dd, J=8.2, 2.3 Hz, 1H), 7.37 (d, J=8.4 Hz, 1H), 6.98 (s, 1H), 6.63 (s, 1H), 2.87 (s, 3H), 2.29 (s, 3H). MS (ESI) m/z 456.0 [M+H]⁺

### Example 55. Synthesis of 2-(2-Cyanopropan-2-yl)-N-(6-methyl-5-(7-(methylamino)-1,6-naphthyridin-3-yl)pyridin-3-yl)isonicotinamide (Compound 55)

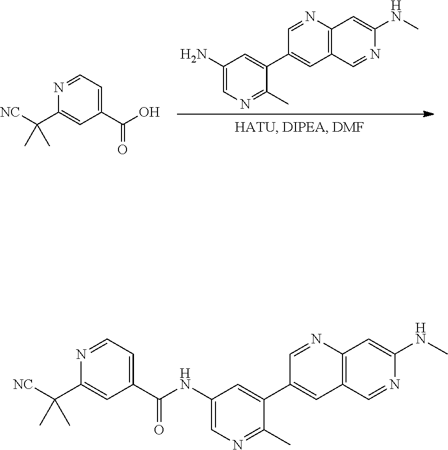 [SMILES: CC(C)(C#N)c1cc(C(=O)O)ccn1 ; CNc1cc2ncc(-c3cc(N)cnc3C)cc2cn1 ; CNc1cc2ncc(-c3cc(NC(=O)c4ccnc(C(C)(C)C#N)c4)cnc3C)cc2cn1]

A solution of 3-(5-amino-2-methylpyridin-3-yl)-N-methyl-1,6-naphthyridin-7-amine (48 mg, 0.18 mmol), 2-(2-cyanopropan-2-yl)isonicotinic acid (38 mg, 0.2 mmol), 1-[bis(dimethylamino)methylene]-1H-1,2,3-triazolo[4,5-b]pyridinium 3-oxid hexafluorophosphate (114 mg, 0.3 mmol) and N,N-diisopropylethylamine (77 mg, 0.6 mmol) in N,N-dimethylformamide (2 mL) was stirred at 25° C. for 1 hour. The mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 2-(2-cyanopropan-2-yl)-N-(6-methyl-5-(7-(methylamino)-1,6-naphthyridin-3-yl)pyridin-3-yl)isonicotinamide (10 mg, 0.023 mmol, 13%) as a yellow solid. ¹H NMR (500 MHz, DMSO-d₆) δ 10.81 (s, 1H), 9.00 (s, 1H), 8.89 (d, J=2.4 Hz, 1H), 8.86 (d, J=2.3 Hz, 1H), 8.84 (d, J=5.1 Hz, 1H), 8.32 (d, J=2.0 Hz, 1H), 8.15 (d, J=2.4 Hz, 1H), 8.05 (s, 1H), 7.90 (dd, J=5.1, 1.4 Hz, 1H), 6.95 (q, J=4.9 Hz, 1H), 6.64 (s, 1H), 2.88 (d, J=5.0 Hz, 3H), 2.50 (s, 3H), 1.78 (s, 6H). MS (ESI) m/z 438.1 [M+H]⁺

### Example 56. Synthesis of 2-(2-Cyanopropan-2-yl)-N-(4-methyl-3-(7-(methylamino)-1,6-naphthyridin-3-yl)phenyl)isonicotinamide (Compound 56)

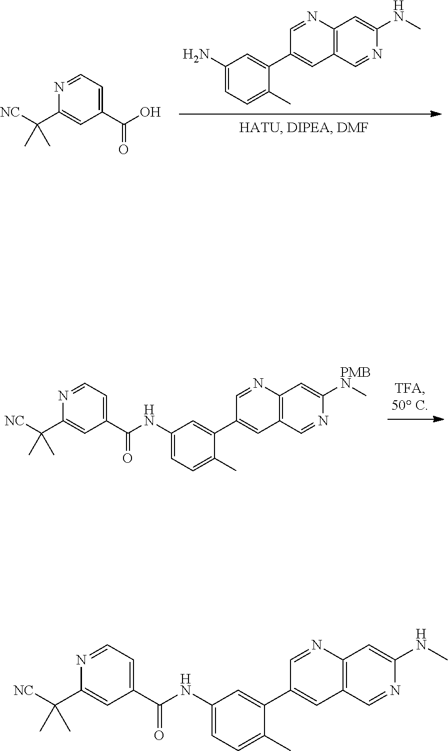

Step 1. To a solution of 2-(2-cyanopropan-2-yl)isonicotinic acid (67 mg, 0.35 mmol) in N,N-dimethylformamide (3 mL) were added 3-(5-amino-2-methylphenyl)-N-(4-methoxybenzyl)-N-methyl-1,6-naphthyridin-7-amine (135 mg, 0.35 mmol), 1-[bis(dimethylamino)methylene]-1H-1,2,3-triazolo[4,5-b]pyridinium 3-oxid hexafluorophosphate (200 mg, 0.53 mmol) and N,N-diisopropylethylamine (90 mg, 0.70 mmol) at room temperature. After stirring at room temperature for 2 h, the mixture was diluted with water (30 mL) and extracted with ethyl acetate (50 mL×2). The combined organic layers were washed with brine (30 mL×3), dried over anhydrous sodium sulfate and filtered. The filtrate was concentrated under reduced pressure. The residue was purified by flash chromatography (silica, ethyl acetate/petroleum ether=1/3) to afford 2-(2-cyanopropan-2-yl)-N-(3-(7-((4-methoxybenzyl)(methyl)amino)-1,6-naphthyridin-3-yl)-4-methylphenyl)isonicotinamide (130 mg, 0.234 mmol, 67%) as a yellow solid. MS (EST) m/z 557.3 [M+H]⁺

Step 2. A solution of 2-(2-cyanopropan-2-yl)-N-(3-(7-((4-methoxybenzyl)(methyl)amino)-1,6-naphthyridin-3-yl)-4-methylphenyl)isonicotinamide (130 mg, 0.234 mmol) in trifluoroacetic acid (5 mL) was stirred at 50° C. for 1 hour. After cooling to room temperature, the mixture concentrated. The residue was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford 2-(2-cyanopropan-2-yl)-N-(4-methyl-3-(7-(methylamino)-1,6-naphthyridin-3-yl)phenyl)isonicotinamide (25.4 mg, 0.058 mmol, 25%) as a yellow solid. ¹H NMR (400 MHz, DMSO-d₆) δ 10.60 (s, 1H), 8.99 (s, 1H), 8.82 (t, J=4.0 Hz, 2H), 8.26 (s, 1H), 8.02 (s, 1H), 7.88 (d, J=5.1 Hz, 1H), 7.76 (d, J=5.9 Hz, 2H), 7.38 (d, J=9.0 Hz, 1H), 6.92 (s, 1H), 6.63 (s, 1H), 2.87 (d, J=4.5 Hz, 3H), 2.30 (s, 3H), 1.77 (s, 6H). MS (ESI) m/z 437.2 [M+H]⁺

### Example 57. Synthesis of 2-Isopropyl-N-(6-methyl-5-(7-(methylamino)-1,6-naphthyridin-3-yl)pyridin-3-yl)isonicotinamide (Compound 57)

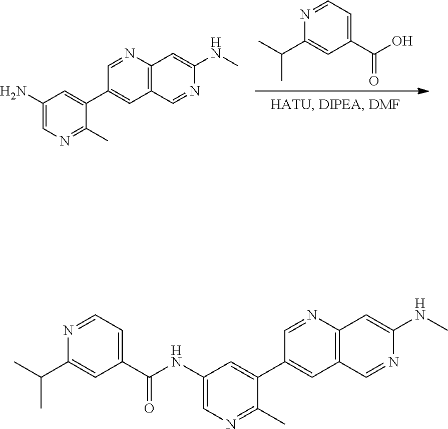

The mixture of 2-isopropylisonicotinic acid (30 mg, 0.18 mmol), 3-(5-amino-2-methylpyridin-3-yl)-N-methyl-1,6-naphthyridin-7-amine (48 mg, 0.18 mmol), 1-[bis(dimethylamino)methylene]-1H-1,2,3-triazolo[4,5-b]pyridinium 3-oxid hexafluorophosphate (103.6 mg, 0.27 mmol) and N,N-diisopropylethylamine (47 mg, 0.36 mmol) in N,N-dimethylformamide (3 mL) was stirred at room temperature for 2 hours. The reaction mixture was concentrated. The residue was purified by prep-HPLC (Column: Sunfire Xbridge 4.6×50 mm C18, 3.5 μm, Mobile Phase: A: 0.01% aqueous ammonium bicarbonate, B: acetonitrile; B %: 5%-95% in 1.5 min) to afford 2-isopropyl-N-(6-methyl-5-(7-(methylamino)-1,6-naphthyridin-3-yl)pyridin-3-yl)isonicotinamide (15.1 mg, 0.04 mmol, 20%) as a yellow solid. ¹H NMR (500 MHz, DMSO-d₆) δ 10.70 (s, 1H), 8.99 (s, 1H), 8.88 (dd, J=13.2, 2.3 Hz, 2H), 8.71 (d, J=5.1 Hz, 1H), 8.32 (d, J=2.2 Hz, 1H), 8.16 (d, J=2.4 Hz, 1H), 7.77 (s, 1H), 7.70 (dd, J=5.1, 1.5 Hz, 1H), 6.96 (d, J=5.0 Hz, 1H), 6.63 (s, 1H), 3.14 (dt, J=13.8, 6.9 Hz, 1H), 2.87 (d, J=5.0 Hz, 3H), 2.51 (s, 3H), 1.30 (s, 3H), 1.28 (s, 3H). MS (ESI) m/z 412.8 [M+H]⁺

### Example 58. Synthesis of 2-(tert-Butyl)-N-(6-methyl-5-(7-(methylamino)-1,6-naphthyridin-3-yl)pyridin-3-yl)isonicotinamide (Compound 58)

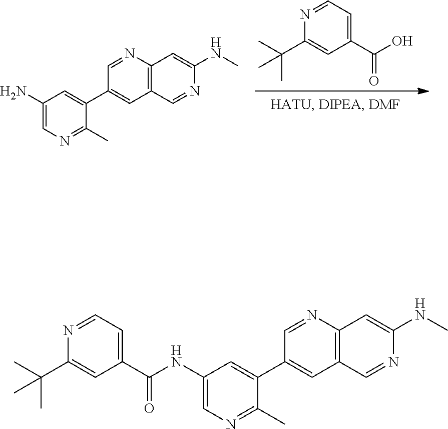

The mixture of 2-(tert-butyl)isonicotinic acid (30 mg, 0.17 mmol), 3-(5-amino-2-methylpyridin-3-yl)-N-methyl-1,6-naphthyridin-7-amine (44 mg, 0.17 mmol), 1-[bis(dimethylamino)methylene]-1H-1,2,3-triazolo[4,5-b]pyridinium 3-oxid hexafluorophosphate (95.5 mg, 0.25 mmol) and N,N-diisopropylethylamine (43.2 mg, 0.34 mmol) in N,N-dimethylformamide (3 mL) was stirred at room temperature for 2 hours. The reaction mixture was concentrated. The residue was purified by prep-HPLC (Column: Sunfire Xbridge 4.6×50 mm C18, 3.5 μm, Mobile Phase: A: 0.01% aqueous ammonium bicarbonate, B: acetonitrile; B %: 5%-95% in 1.5 min) to afford 2-(tert-butyl)-N-(6-methyl-5-(7-(methylamino)-1,6-naphthyridin-3-yl)pyridin-3-yl)isonicotinamide (16.9 mg, 0.04 mmol, 24%) as a yellow solid. ¹H NMR (400 MHz, DMSO-d₆) δ 10.70 (s, 1H), 9.00 (s, 1H), 8.88 (dd, J=10.4, 2.3 Hz, 2H), 8.74 (d, J=5.0 Hz, 1H), 8.32 (d, J=2.1 Hz, 1H), 8.15 (d, J=2.3 Hz, 1H), 7.89 (s, 1H), 7.71 (d, J=5.0 Hz, 1H), 6.96 (d, J=4.9 Hz, 1H), 6.64 (s, 1H), 2.88 (d, J=4.9 Hz, 3H), 2.51 (s, 3H), 1.38 (s, 9H). MS (EST) m/z 426.8 [M+H]⁺

### Example 59. Synthesis of N-(4-(2-Cyanopropan-2-yl)pyridin-2-yl)-6-methyl-5-(7-(methylamino)-1,6-naphthyridin-3-yl)nicotinamide (Compound 59)

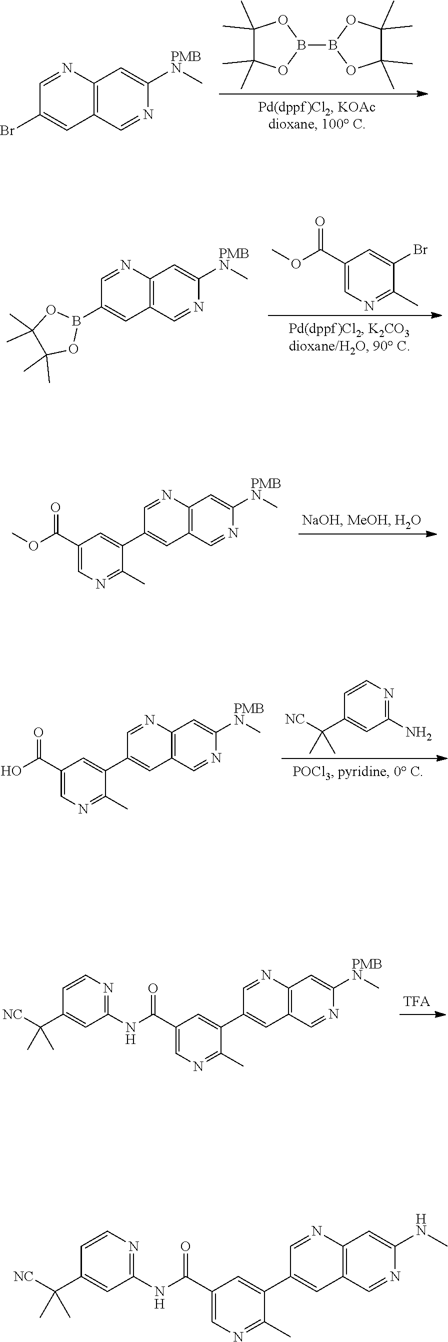

Step 1. [1,1′-Bis(diphenylphosphino)ferrocene]dichloropalladium(II) (21 mg, 0.029 mmol) was added to a mixture of 3-bromo-N-(4-methoxybenzyl)-N-methyl-1,6-naphthyridin-7-amine (100 mg, 0.28 mmol), 4,4,4′,4′,5,5,5′,5′-octamethyl-2,2′-bi(1,3,2-dioxaborolane) (152 mg, 0.6 mmol) and potassium acetate (245 mg, 0.25 mmol) in dioxane (5 mL) at room temperature. The reaction mixture was stirred at 100° C. for 1 hour under nitrogen. After cooling to room temperature, the reaction was diluted with ethyl acetate (20 mL), washed with water (20 mL) and brine (20 mL), dried over sodium sulfate, filtered and concentrated to afford N-(4-methoxybenzyl)-N-methyl-3-(4,4,5,5-tetramethyl-1,3,2-dioxaborolan-2-yl)-1,6-naphthyridin-7-amine (100 mg, 0.25 mmol, 89%) as a yellow solid, which was used without further purification. MS (ESI) m/z 406.2 [M+H]⁺

Step 2. [1,1′-Bis(diphenylphosphino)ferrocene]dichloropalladium(II) (21 mg, 0.029 mmol) was added to a solution of methyl 5-bromo-6-methylnicotinate (58 mg, 0.25 mmol), N-(4-methoxybenzyl)-N-methyl-3-(4,4,5,5-tetramethyl-1,3,2-dioxaborolan-2-yl)-1,6-naphthyridin-7-amine (100 mg, 0.25 mmol) and potassium carbonate (104 mg, 0.75 mmol) in dioxane (10 mL) and water (2 mL) at room temperature. The reaction solution was stirred at 90° C. for 2 hours under nitrogen. After cooling to room temperature, the reaction was diluted with ethyl acetate (20 mL), washed with water (20 mL) and brine (20 mL), dried over sodium sulfate, filtered, and concentrated. The residue was purified by flash chromatography (silica, petroleum ether/ethyl acetated=2/1 then 1/1) to afford methyl 5-(7-((4-methoxybenzyl)(methyl)amino)-1,6-naphthyridin-3-yl)-6-methylnicotinate (80 mg, 0.187 mmol, 75%) as a yellow solid. MS (EST) m/z 429.1 [M+H]⁺

Step 3. A solution of methyl 5-(7-((4-methoxybenzyl)(methyl)amino)-1,6-naphthyridin-3-yl)-6-methylnicotinate (80 mg, 0.187 mmol) and sodium hydroxide (84 mg, 2.11 mmol) in water (5 mL) and methanol (5 mL) was stirred at room temperature for 3 hours under Argon. The reaction mixture was adjusted to pH=3.0 with 2 N hydrogen chloride. The reaction mixture was concentrated, diluted with ethyl acetate (25 mL), and washed with water (25 mL). The organic phase was washed with brine (25 mL), dried over sodium sulfate, filtered, and concentrated. The residue was purified by flash chromatography (silica, dichloromethane/methanol=20/1) to afford 5-(7-((4-methoxybenzyl)(methyl)amino)-1,6-naphthyridin-3-yl)-6-methylnicotinic acid (50 mg, 0.12 mmol, 64%) as a yellow solid. MS (ESI) m/z 415.3 [M+H]⁺

Step 4. To a solution of 5-(7-((4-methoxybenzyl)(methyl)amino)-1,6-naphthyridin-3-yl)-6-methylnicotinic acid (50 mg, 0.12 mmol) in pyridine (3 mL) was added 2-(2-aminopyridin-4-yl)-2-methylpropanenitrile (19 mg, 0.12 mmol) and phosphoryl trichloride (97 mg, 0.63 mmol) at 0° C. The reaction mixture was stirred at 0° C. for 1 hour. The reaction mixture was poured to water at 0° C., and then extracted with ethyl acetate (30 mL). The organic layer was washed with water (20 mL×3) and brine (20 mL), dried over sodium sulfate, filtered and concentrated. The residue was purified by flash chromatography (silica, ethyl acetate) to afford N-(4-(2-cyanopropan-2-yl)pyridin-2-yl)-5-(7-((4-methoxybenzyl)(methyl)amino)-1,6-naphthyridin-3-yl)-6-methylnicotinamide (30 mg, 0.054 mmol, 45%) as a yellow solid. MS (ESI) m/z 557.7 [M+H]⁺

Step 5. N-(4-(2-cyanopropan-2-yl)pyridin-2-yl)-5-(7-((4-methoxybenzyl)(methyl)amino)-1,6-naphthyridin-3-yl)-6-methylnicotinamide (30 mg, 0.054 mmol) in trifluoroacetic acid (2 mL) was stirred at room temperature for 1 hour. The reaction mixture was concentrated and the residue was purified by prep-HPLC (Column: Waters Xbridge 21.2×250 mm C18, 10 μm, Mobile Phase: A: water (10 mmol/L ammonium bicarbonate), B: acetonitrile) to afford N-(4-(2-cyanopropan-2-yl)pyridin-2-yl)-6-methyl-5-(7-(methylamino)-1,6-naphthyridin-3-yl)nicotinamide (3.3 mg, 0.0076 mmol, 14%) as a yellow solid. ¹H NMR (400 MHz, DMSO-d₆) δ 11.28 (s, 1H), 9.07 (d, J=1.2 Hz, 1H), 9.06-8.97 (m, 2H), 8.49-0.40 (m, 4H), 7.35-7.34 (m, 1H), 6.98 (d, J=5.2 Hz, 1H), 6.65 (s, 1H), 2.88 (d, J=5.2 Hz, 3H), 2.62 (s, 3H) 1.73 (s, 6H). MS (EST) m/z 438.1 [M+H]⁺

### Example 60. Synthesis of 5-Fluoro-N-(6-methyl-5-(7-(methylamino)-1,6-naphthyridin-3-yl)pyridin-3-yl)-4-(trifluoromethyl)picolinamide (Compound 60)

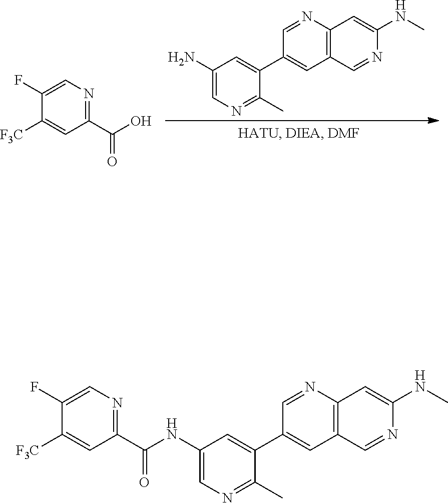 [SMILES: O=C(O)c1cc(C(F)(F)F)c(F)cn1 ; CNc1cc2ncc(-c3cc(N)cnc3C)cc2cn1 ; CNc1cc2ncc(-c3cc(NC(=O)c4cc(C(F)(F)F)c(F)cn4)cnc3C)cc2cn1]

A mixture of 5-fluoro-4-(trifluoromethyl)picolinic acid (60 mg, 0.29 mmol), 3-(5-amino-2-methylpyridin-3-yl)-N-methyl-1,6-naphthyridin-7-amine (76 mg, 0.29 mmol), 1-[bis(dimethylamino)methylene]-1H-1,2,3-triazolo[4,5-b]pyridinium 3-oxid hexafluorophosphate (131 mg, 0.34 mmol) and N,N-diisopropylethylamine (74 mg, 0.57 mmol) in N,N-dimethylformamide (3 mL) was stirred at room temperature for 2 hours. The mixture was purified by prep-HPLC (Column: Waters Xbridge 21.2×250 mm C18, 10 μm, Mobile Phase: A: water (10 mmol/L ammonium bicarbonate), B: acetonitrile) to afford 5-fluoro-N-(6-methyl-5-(7-(methylamino)-1,6-naphthyridin-3-yl)pyridin-3-yl)-4-(trifluoromethyl)picolinamide (15.6 mg, 0.03 mmol, 49%) as a yellow solid. ¹H NMR (400 MHz, DMSO-d₆) δ 11.09 (s, 1H), 9.01 (s, 1H), 8.93 (d, J=2.4 Hz, 1H), 8.89 (d, J=2.4 Hz, 1H), 8.84 (d, J=4.4 Hz, 1H), 8.36 (s, 1H), 8.24 (d, J=2.4 Hz, 1H), 8.16 (t, J=5.2 Hz, 1H), 7.01 (s, 1H), 6.64 (s, 1H), 2.88 (d, J=3.6 Hz, 3H), 2.55 (s, 3H). MS (ESI) m/z 457.0 [M+H]⁺

### Example 61. Synthesis of N-(4-Methyl-3-(7-(methylamino)-1,6-naphthyridin-3-yl)phenyl)-4-(trifluoromethyl)picolinimidamide (Compound 61)

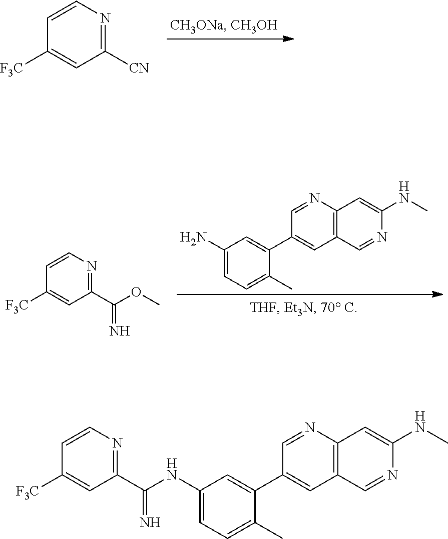 [SMILES: N#Cc1cc(C(F)(F)F)ccn1 ; COC(=N)c1cc(C(F)(F)F)ccn1 ; CNc1cc2ncc(-c3cc(N)ccc3C)cc2cn1 ; CNc1cc2ncc(-c3cc(NC(=N)c4cc(C(F)(F)F)ccn4)ccc3C)cc2cn1]

Step 1. A solution of 4-(trifluoromethyl)picolinonitrile (400 mg, 2.91 mmol) and sodium methoxide (30% in methanol) in methanol (5 mL) was stirred at room temperature for 16 hours. The mixture was diluted with water and extracted with ethyl acetate (30 mL×3). The combined organic phases were dried over sodium sulfate, filtered, and concentrated under reduced pressure to afford methyl 4-(trifluoromethyl)picolinimidate (352 mg, 1.73 mmol, 59%) as a colorless oil. MS (ESI) m/z 204.9 [M+H]⁺

Step 2. A solution of methyl 4-(trifluoromethyl)picolinimidate (70 mg, 0.34 mmol), 3-(5-amino-2-methylphenyl)-N-methyl-1,6-naphthyridin-7-amine (90 mg, 0.34 mmol) and triethylamine (0.2 mL) in tetrahydrofuran (3 mL) was stirred at 70° C. for 3 hours. After cooling to room temperature, the resulting mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford N-(4-methyl-3-(7-(methylamino)-1,6-naphthyridin-3-yl)phenyl)-4-(trifluoromethyl)picolinimidamide (6.3 mg, 0.014 mmol, 4%) as a yellow solid. ¹H NMR (400 MHz, DMSO-d₆) δ 8.98-8.92 (m, 2H), 8.86 (d, J=2.3 Hz, 1H), 8.55 (s, 1H), 8.25 (d, 1=1.8 Hz, 1H), 7.97 (d, 1=4.9 Hz, 1H), 7.34 (d, J=8.3 Hz, 2H), 6.94 (s, 2H), 6.87 (d, J=5.0 Hz, 1H), 6.61 (s, 1H), 2.86 (d, 1=4.9 Hz, 3H), 2.30 (s, 3H). MS (ESI) m/z 437.0 [M+H]⁺

### Example 62. Synthesis of (S)—N-(6-Methyl-5-(7-(methylamino)-1,6-naphthyridin-3-yl)pyridin-3-yl)-2-(3-(trifluoromethyl)piperidin-1-yl)acetamide (Compound 62)

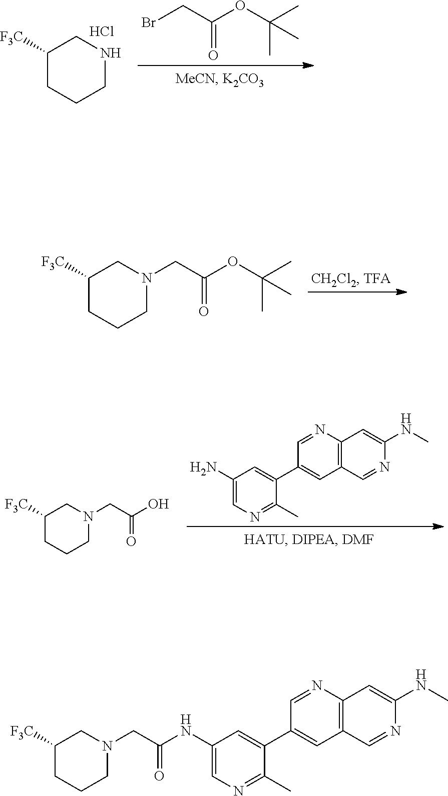

Step 1. To a mixture of (S)-3-(trifluoromethyl)piperidine hydrochloride (19 mg, 0.1 mmol) and potassium carbonate (69 mg, 0.5 mmol) in anhydrous acetonitrile (3 mL) was added tert-butyl 2-bromoacetate (20 mg, 0.1 mmol). Then the reaction mixture was stirred at 25° C. for 16 hours. The mixture was diluted with acetonitrile and filtered. The filtrate was concentrated under reduced pressure to afford (S)-tert-butyl 2-(3-(trifluoromethyl)piperidin-1-yl)acetate (26 mg) as a yellow oil. The crude product was used in the next step without further purification. MS (ESI) m/z 267.9 [M+H]⁺

Step 2. To a mixture of (S)-tert-butyl 2-(3-(trifluoromethyl)piperidin-1-yl)acetate (26 mg, 0.1 mmol) in dichloromethane (1 mL) was added trifluoroacetic acid (1 mL). The mixture was stirred at 25° C. for 16 hours and concentrated to afford (S)-2-(3-(trifluoromethyl)piperidin-1-yl)acetic acid (21 mg) as a yellow oil, which was used in the next step without further purification. MS (EST) m/z 212.3 [M+H]⁺

Step 3. To a solution of crude (S)-2-(3-(trifluoromethyl)piperidin-1-yl)acetic acid (21 mg, 0.1 mmol), 3-(5-amino-2-methylpyridin-3-yl)-N-methyl-1,6-naphthyridin-7-amine (21 mg, 0.08 mmol) and 1-[bis(dimethylamino)methylene]-1H-1,2,3-triazolo[4,5-b]pyridinium 3-oxid hexafluorophosphate (61 mg, 0.16 mmol) in N,N-dimethylformamide (2 ml) was added N,N-diisopropylethylamine (41 mg, 0.32 mmol). The mixture was stirred at 25° C. for 1 hour. The mixture purified by prep-HPLC (Column: Welch Xtimate 21.2×250 mm C18, 10 μm, Mobile Phase. A: water (10 mmol/L ammonium bicarbonate), B: acetonitrile; B %: 30%-70% in 15 min) to afford (S)—N-(6-methyl-5-(7-(methylamino)-1,6-naphthyridin-3-yl)pyridin-3-yl)-2-(3-(trifluoromethyl)piperidin-1-yl)acetamide (18.6 mg, 0.049 mmol, 41%) as a yellow solid. ¹H NMR (400 MHz, DMSO-d₆) δ 9.95 (s, 1H), 8.98 (s, 1H), 8.83 (d, J=2.3 Hz, 1H), 8.76 (d, J=2.4 Hz, 1H), 8.28 (d, J=2.1 Hz, 1H), 8.05 (d, J=2.3 Hz, 1H), 6.95 (q, J=4.9 Hz, 1H), 6.63 (s, 1H), 3.25 (dd, J=31.5, 15.8 Hz, 2H), 3.08-3.01 (m, 1H), 2.87 (d, J=5.0 Hz, 3H), 2.84-2.79 (m, 1H), 2.71-2.57 (m, 1H), 2.45 (s, 3H), 2.27-2.12 (m, 2H), 1.92-1.84 (m, 1H), 1.74-1.55 (m, 2H), 1.30-1.15 (m, 1H). MS (ESI) m/z 458.7 [M+H]⁺

### Example 63. Synthesis of (S)—N-(4-Methyl-3-(7-(methylamino)-1,6-naphthyridin-3-yl)phenyl)-2-(3-(trifluoromethyl)piperidin-1-yl)acetamide (Compound 63)

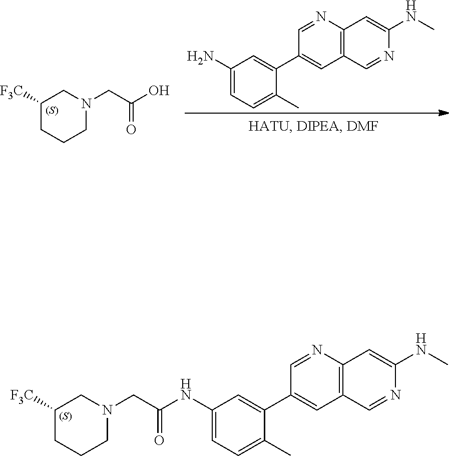

To a solution of (S)-2-(3-(trifluoromethyl)piperidin-1-yl)acetic acid (21 mg, 0.1 mmol), 3-(5-amino-2-methylphenyl)-N-methyl-1,6-naphthyridin-7-amine (21 mg, 0.08 mmol) and 1-[bis(dimethylamino)methylene]-1H-1,2,3-triazolo[4,5-b]pyridinium 3-oxid hexafluorophosphate (61 mg, 0.16 mmol) in N,N-dimethylformamide (2 ml) was added N,N-diisopropylethylamine (41 mg, 0.32 mmol). The mixture was stirred at 25° C. for 1 hour. The mixture purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford (S)—N-(4-methyl-3-(7-(methylamino)-1,6-naphthyridin-3-yl)phenyl)-2-(3-(trifluoromethyl)piperidin-1-yl)acetamide (20.5 mg, 0.049 mmol, 49%) as a yellow solid. ¹H NMR (400 MHz, DMSO-d₆) δ 9.74 (s, 1H), 8.97 (s, 1H), 8.78 (d, J=2.3 Hz, 1H), 8.20 (d, J=1.8 Hz, 1H), 7.73-7.54 (m, 2H), 7.38-7.20 (m, 1H), 6.98-6.82 (m, 1H), 6.62 (s, 1H), 3.20 (dd, J=31.0, 15.5 Hz, 2H), 3.04 (d, J=9.7 Hz, 1H), 2.92-2.78 (m, 4H), 2.70-2.59 (m, 1H), 2.39-2.09 (m, 5H), 1.87 (d, J=10.8 Hz, 1H), 1.77-1.49 (m, 2H), 1.29-1.16 (m, 1H). MS (ESI) m/z 458.4 [M+H]⁺

### Example 64. Synthesis of N-(6-Methyl-5-(7-(methylamino)-1,6-naphthyridin-3-yl)pyridin-3-yl)-4-(trifluoromethyl)picolinimidamide (Compound 64)

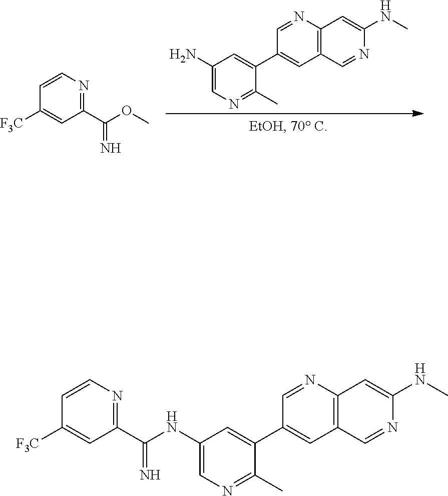 [SMILES: COC(=N)c1cc(C(F)(F)F)ccn1 ; CNc1cc2ncc(-c3cc(N)cnc3C)cc2cn1 ; CNc1cc2ncc(-c3cc(NC(=N)c4cc(C(F)(F)F)ccn4)cnc3C)cc2cn1]

A solution of methyl 4-(trifluoromethyl)picolinimidate (70 mg, 0.34 mmol), 3-(5-amino-2-methylpyridin-3-yl)-N-methyl-1,6-naphthyridin-7-amine (91 mg, 0.34 mmol) in ethanol (3 mL) was stirred at 70° C. for 16 hours. After cooling to room temperature, the resulting mixture was purified by prep-HPLC (Column: Welch Xtimate 21.2×250 mm C18, 10 μm, Mobile Phase: A: water (10 mmol/L ammonium bicarbonate), B: acetonitrile; B %: 30%-70% in 15 min) to afford N-(6-methyl-5-(7-(methylamino)-1,6-naphthyridin-3-yl)pyridin-3-yl)-4-(trifluoromethyl)picolinimidamide (19.0 mg, 0.04 mmol, 13%) as a yellow solid. ¹H NMR (400 MHz, DMSO-d₆) δ 8.98-8.93 (m, 2H), 8.89 (s, 1H), 8.56 (s, 1H), 8.32 (s, 1H), 8.17 (s, 1H), 7.99 (d, J=5.1 Hz, 1H), 7.33 (s, 1H), 7.14 (s, 1H), 6.92 (d, J=4.9 Hz, 1H), 6.62 (s, 1H), 2.87 (d, J=4.8 Hz, 3H), 2.49 (s, 3H). MS (ESI) m/z 438.1 [M+H]⁺

### Example 65. Synthesis of 4-(2-Fluoropropan-2-yl)-N-(6-methyl-5-(7-(methylamino)-1,6-naphthyridin-3-yl)pyridin-3-yl)picolinamide (Compound 65)

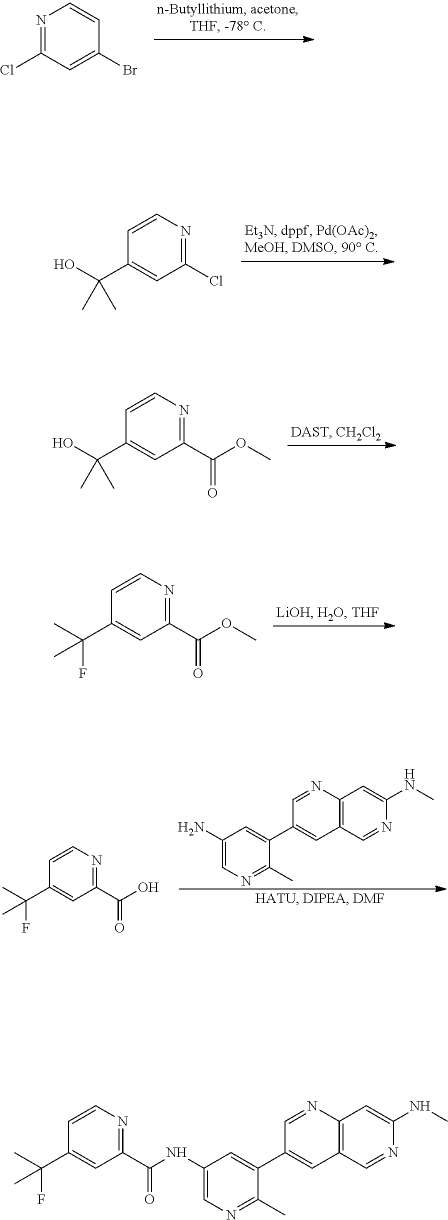

Step 1. n-Butyllithium (8.7 mL, 17.4 mmol) was added to a solution of 4-bromo-2-chloropyridine (3000 mg, 15.7 mmol) in tetrahydrofuran (30 mL) at −78° C. After stirring −78° C. for 1 hour under nitrogen, acetone (504 mg, 17.4 mmol) was added. The reaction was allowed to rise to room temperature and concentrated. The residue was purified by flash chromatography (silica, dichloromethane/methanol=20/1) to afford 2-(2-chloropyridin-4-yl)propan-2-ol (1200 mg, 7.01 mmol, 44%) as a white solid. MS (EST) m/z 172 [M+H]⁺

Step 2. Palladium(II)acetate (29 mg, 0.103 mmol) was added to a solution of 1,1′-bis(diphenylphosphino)ferrocene (57 mg, 0.103 mmol), 2-(2-chloropyridin-4-yl)propan-2-ol (2000 mg, 10.3 mmol) and potassium carbonate (231 mg, 1.671 mmol) in dimethyl sulfoxide (10 mL) and methanol (2 mL) at room temperature. The reaction mixture was stirred at 90° C. for 12 hours under nitrogen. After cooling to room temperature, the reaction was diluted with ethyl acetate (60 mL), washed with water (20 mL) and brine (20 mL), dried over sodium sulfate, filtered, and concentrated. The residue was purified by flash chromatography (silica, petroleum ether/ethyl acetated=2/1 then 1/1) to afford methyl 4-(2-hydroxypropan-2-yl)picolinate (400 mg, 2.05 mmol, 20%) as a brown solid. MS (ESI) m/z 196 [M+H]⁺

Step 3. Methyl 4-(2-hydroxypropan-2-yl)picolinate (400 mg, 2.05 mmol) was added to a solution of diethylaminosulfur trifluoride (363 mg, 2.26 mmol) in dichloromethane (10 mL) at 25° C. under nitrogen. After stirring at 25° C. for 2 h, the reaction mixture was diluted with ethyl acetate (80 mL), washed with water (20 mL) and brine (20 mL), dried over sodium sulfate, filtered, and concentrated. The residue was purified by flash chromatography (silica, petroleum ether/ethyl acetated=2/1 then 1/1) to afford methyl 4-(2-fluoropropan-2-yl)picolinate (300 mg, 1.52 mmol, 74%) as a brown solid. MS (ESI) m/z 198 [M+H]⁺

Step 4. Methyl 4-(2-fluoropropan-2-yl)picolinate (300 mg, 1.64 mmol) was added to a solution of lithium hydroxide (141 mg, 3.28 mmol), water (3 mL) and tetrahydrofuran (6 mL) at 25° C. After stirring at 25° C. for 2 h, dilute hydrochloric acid (3 mL, 3.28 mmol) was added to the reaction solution. The resulting reaction solution was diluted with ethyl acetate (60 mL), washed with water (20 mL) and brine (20 mL), dried over sodium sulfate, filtered, and concentrated. The residue was purified by prep-HPLC (Column: Waters Xbridge 21.2×250 mm C18, 10 μm, Mobile Phase: A: water (10 mmol/L ammonium bicarbonate), B: acetonitrile) to afford 4-(2-fluoropropan-2-yl)picolinic acid (240 mg, 1.24 mmol, 75%) as a yellow solid. MS (ESI) m/z 184 [M+H]⁺

Step 5. To a solution of 4-(2-fluoropropan-2-yl)picolinic acid (20 mg, 0.11 mmol), 3-(5-amino-2-methylpyridin-3-yl)-N-methyl-1,6-naphthyridin-7-amine (40 mg, 0.15 mmol) and 1-[bis(dimethylamino)methylene]-1H-1,2,3-triazolo[4,5-b]pyridinium 3-oxid hexafluorophosphate (57 mg, 0.15 mmol) in N,N-dimethylformamide (2 mL) was added N,N-diisopropylethylamine (0.2 mL) at room temperature. After stirring at room temperature for 16 h, the reaction mixture was concentrated. The residue was purified by prep-HPLC (Column: Waters Xbridge 21.2×250 mm C18, 10 μm, Mobile Phase: A: water (10 mmol/L ammonium bicarbonate), B: acetonitrile) to afford 4-(2-fluoropropan-2-yl)-N-(6-methyl-5-(7-(methylamino)-1,6-naphthyridin-3-yl)pyridin-3-yl)picolinamide (15 mg, 0.034 mmol, 33%) as a yellow solid. ¹H NMR (400 MHz, CD₃OD) δ 8.97 (d, J=13.3 Hz, 2H), 8.84 (s, 1H), 8.71 (d, J=7 Hz, 1H), 8.33 (d, J=11.1 Hz, 2H), 8.24 (s, 1H), 7.65 (d, J=4.4 Hz, 1H), 6.72 (s, 1H), 2.99 (s, 3H), 2.55 (s, 3H), 1.70 (dd, J=21.9, 12.1 Hz, 3H), 1.30 (d, J=5.5 Hz, 3H). MS (ESI) m/z 431.2 [M+H]⁺

### Example 66. Synthesis of 4-(Difluoromethyl)-5-fluoro-N-(6-methyl-5-(7-(methylamino)-1,6-naphthyridin-3-yl)pyridin-3-yl)picolinamide (Compound 66)

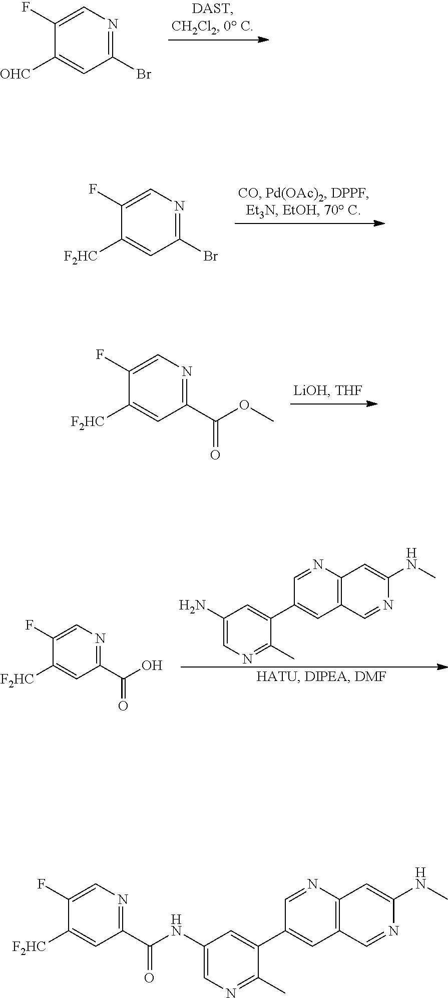 [SMILES: O=Cc1cc(Br)ncc1F ; Fc1cnc(Br)cc1C(F)F ; COC(=O)c1cc(C(F)F)c(F)cn1 ; O=C(O)c1cc(C(F)F)c(F)cn1 ; CNc1cc2ncc(-c3cc(N)cnc3C)cc2cn1 ; CNc1cc2ncc(-c3cc(NC(=O)c4cc(C(F)F)c(F)cn4)cnc3C)cc2cn1]

Step 1. A solution of 2-bromo-5-fluoroisonicotinaldehyde (500 mg, 2.47 mmol), diethylaminosulfur trifluoride (1992 mg, 12.35 mmol) in dichloromethane (8 mL) was stirred at 0° C. for 2 hours. The mixture was diluted with water (100 mL) and extracted with ethyl acetate (100 mL×3). The combined organic layers were washed with brine (10 mL), dried over anhydrous sodium sulfate, filtered and concentrated. The residue was purified by flash chromatography (silica, petroleum ether/ethyl acetate=1/1) to afford 2-bromo-4-(difluoromethyl)-5-fluoropyridine (400 mg, 1.78 mmol, 72%) as a yellow solid. MS (ESI) m/z 225.8 [M+H]⁺

Step 2. A mixture of 2-bromo-4-(difluoromethyl)-5-fluoropyridine (400 mg, 1.78 mmol), palladium(1H)acetate (241 mg, 4.31 mmol), 1,1′-bis(diphenylphosphino)ferrocene (242 mg, 4.31 mmol) and triethylamine (241 mg, 4.31 mmol) in methanol (6 mL) was stirred at 70° C. for 16 hours under carbon monoxide. After cooling to room temperature, the reaction mixture was diluted with water (100 mL) and extracted with ethyl acetate (100 mL×3). The combined organic phases were washed brine (100 mL×2), dried over sodium sulfate, filtered, and concentrated. The residue was purified by flash chromatography (silica, petroleum ether/ethyl acetate=1/2) to afford methyl 4-(difluoromethyl)-5-fluoropicolinate (300 mg, 1.03 mmol, 79%) as a yellow solid. MS (ESI) m/z 206.1 [M+H]⁺

Step 3. A solution of methyl 4-(difluoromethyl)-5-fluoropicolinate (300 mg, 1.03 mmol) and lithium hydroxide (159 mg, 5.15 mmol) in tetrahydrofuran (5 mL) was stirred at room temperature for 2 hours. The reaction mixture was diluted with water (20 mL) and extracted with ethyl acetate (10 mL×3). The combined organic phases were washed brine (10 mL×2), dried over sodium sulfate, filtered, and concentrated. The residue was purified by prep-HPLC (Column: Waters Xbridge 21.2×250 mm C18, 10 μm, Mobile Phase: A: water (10 mmol/L ammonium bicarbonate), B: acetonitrile) to afford 4-(difluoromethyl)-5-fluoropicolinic acid (100 mg, 0.52 mmol, 39%) as a yellow solid. MS (ESI) m/z 192.1 [M+H]⁺

Step 4. A solution of 4-(difluoromethyl)-5-fluoropicolinic acid (100 mg, 0.52 mmol), 3-(5-amino-2-methylpyridin-3-yl)-N-methyl-1,6-naphthyridin-7-amine (138 mg, 0.52 mmol), 1-[bis(dimethylamino)methylene]-1H-1,2,3-triazolo[4,5-b]pyridinium 3-oxid hexafluorophosphate (296 mg, 0.78 mmol) and N,N-diisopropylethylamine (217 mg, 1.56 mmol) in N,N-dimethylformamide (5 mL) was stirred at room temperature for 2 hours. The mixture was quenched with brine (20 mL) and extracted with ethyl acetate (10 mL×3). The combined organic phases were dried over sodium sulphate, filtered and concentrated under reduced pressure to afford 4-(difluoromethyl)-5-fluoro-N-(6-methyl-5-(7-(methylamino)-1,6-naphthyridin-3-yl)pyridin-3-yl)picolinamide (16 mg, 0.18 mmol, 7%) as a yellow solid. ¹H NMR (400 MHz, DMSO-d₆) δ 11.06 (s, 1H), 9.10-9.00 (m, 2H), 8.97 (s, 1H), 8.89 (d, J=2.3 Hz, 1H), 8.44-8.28 (m, 3H), 7.43 (t, J=53.3 Hz, 1H), 7.08 (s, 1H), 6.64 (s, 1H), 2.88 (s, 3H), 2.51 (s, 3H). MS (ESI) m/z 438.8 [M+H]⁺

### Example 67. Synthesis of 5-(2-Cyanopropan-2-yl)-N-(6-methyl-5-(7-(methylamino)-1,6-naphthyridin-3-yl)pyridin-3-yl)nicotinamide (Compound 67)

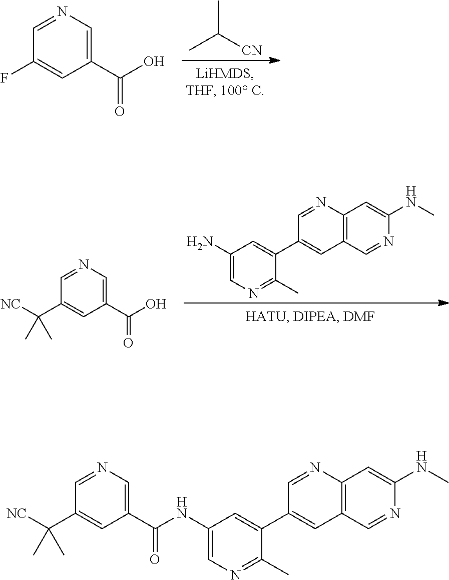

Step 1. Lithium hexamethyldisilazide (1.9 mL, 1.6 M in tetrahydrofuran, 3.0 mmol) was added to a mixture of 5-fluoronicotinic acid (141 mg, 1.0 mmol) and isobutyronitrile (0.26 mL, 3.0 mmol) in tetrahydrofuran (2 mL) at room temperature. After stirring at 100° C. for 0.5 h, the reaction mixture was quenched with saturated ammonium chloride solution (10 mL) and adjusted to pH=4 with 6 N hydrochloric acid. The mixture was extracted with ethyl acetate (10 mL×3). The combined organic layers were washed with brine (20 mL), dried over sodium sulfate, filtered and concentrated to afford 5-(2-cyanopropan-2-yl)nicotinic acid (180 mg, 0.95 mmol, 95%) as a white solid. MS (ESI) m/z 191.4 [M+H]⁺

Step 2. To an N,N-dimethylformamide (3 mL) solution of 3-(5-amino-2-methylpyridin-3-yl)-N-methyl-1,6-naphthyridin-7-amine (80 mg, 0.3 mmol), 5-(2-cyanopropan-2-yl)nicotinic acid (60 mg, 0.3 mmol) and 1-[bis(dimethylamino)methylene]-1H-1,2,3-triazolo[4,5-b]pyridinium 3-oxid hexafluorophosphate (152 mg, 0.4 mmol) was added N,N-diisopropylethylamine (0.25 mL). The mixture was stirred at room temperature for 1 hour. The mixture was purified by prep-HPLC (column: Waters Xbridge 25×150 mm, 5 μm, Mobile phase: A: water (0.05% ammonia hydroxide), B: acetonitrile; B %: 30%-57% in 10 min) to afford 5-(2-cyanopropan-2-yl)-N-(6-methyl-5-(7-(methylamino)-1,6-naphthyridin-3-yl)pyridin-3-yl)nicotinamide (89.7 mg, 0.205 mmol, 68%) as a yellow solid. ¹H NMR (500 MHz, DMSO-d₆) δ 10.76 (s, 1H), 9.14 (d, J=2.0 Hz, 1H), 9.00 (s, 1H), 8.98 (d, J=2.4 Hz, 1H), 8.89 (d, J=2.5 Hz, 1H), 8.87 (d, J=2.4 Hz, 1H), 8.42 (t, J=2.2 Hz, 1H), 8.32 (d, J=2.0 Hz, 1H), 8.15 (d, J=2.4 Hz, 1H), 6.96 (d, J=5.0 Hz, 1H), 6.64 (s, 1H), 2.88 (d, J=4.9 Hz, 3H), 2.48 (s, 3H), 1.81 (s, 6H). MS (ESI) m/z 438.3 [M+H]⁺

### Examples 68 and 69. Synthesis of (S)—N-(4-Methyl-3-(7-(methylamino)-1,6-naphthyridin-3-yl)phenyl)-2-(3-(trifluoromethyl)-1H-1,2,4-triazol-1-yl)propanamide and (R)—N-(4-Methyl-3-(7-(methylamino)-1,6-naphthyridin-3-yl)phenyl)-2-(3-(trifluoromethyl)-1H-1,2,4-triazol-1-yl)propanamide (Compound 68 and Compound 69)

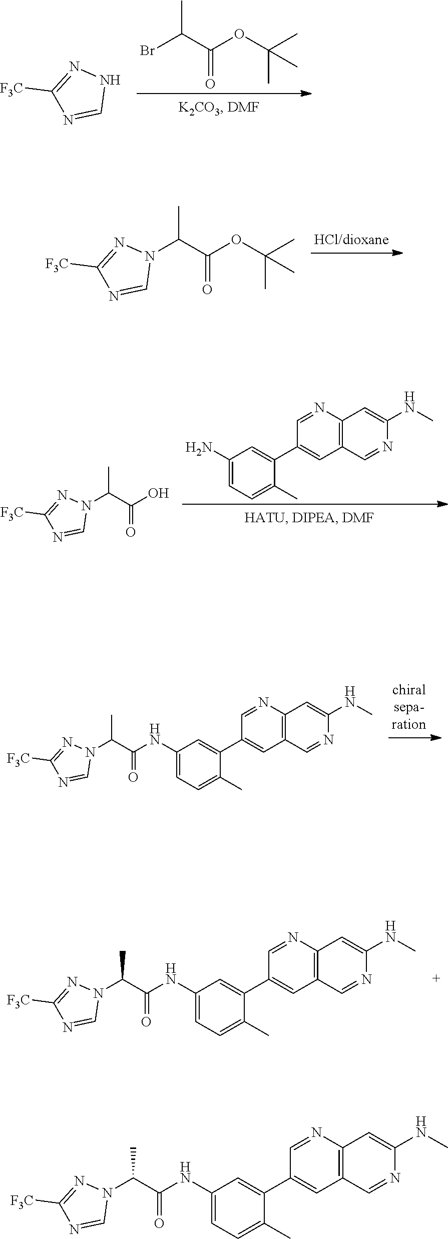

Step 1. To a mixture of 3-(trifluoromethyl)-1H-1,2,4-triazole (100 mg, 0.73 mmol) and tert-butyl 2-bromopropanoate (182 mg, 0.88 mmol) in N,N-dimethylformamide (5 mL) was added potassium carbonate (201 mg, 1.46 mmol). After stirring at room temperature for 2 h, the reaction mixture was quenched with water (30 mL) and extracted with dichloromethane (30 mL×3). The combined organic layers were dried over anhydrous sodium sulfate, filtered and concentrated. The residue was purified by flash chromatography (silica, petroleum ether/ethyl acetate=10/1 to 1/1) to afford tert-butyl 2-(3-(trifluoromethyl)-1H-1,2,4-triazol-1-yl)propanoate (150 mg, 0.57 mmol, 78%) as a light yellow oil. MS (ESI) m/z 266.3 [M+H]⁺

Step 2. To a solution of ter-butyl 2-(3-(trifluoromethyl)-1H-1,2,4-triazol-1-yl)propanoate (150 mg, 0.57 mmol) in 1,4-dioxane (5 mL) was added 4 M hydrochloride in 1,4-dioxane (5 mL). After stirring at room temperature for 16 h, the reaction mixture was concentrated in vacuo to afford 2-(3-(trifluoromethyl)-1H-1,2,4-triazol-1-yl)propanoic acid (110 mg, 0.53 mmol, 92%) as a light yellow oil, which was used in the next step without further purification. MS (ESI) m/z 210.1 [M+H]⁺

Step 3. A solution of 2-(3-(trifluoromethyl)-1H-1,2,4-triazol-1-yl)propanoic acid (140 mg, 0.67 mmol), 3-(5-amino-2-methylphenyl)-N-methyl-1,6-naphthyridin-7-amine (177 mg, 0.67 mmol), 1-[bis(dimethylamino)methylene]-1H-1,2,3-triazolo[4,5-b]pyridinium 3-oxid hexafluorophosphate (304 mg, 0.8 mmol) and N,N-diisopropylethylamine (173 mg, 1.34 mmol) in N,N-dimethylformamide (5 mL) was stirred at room temperature for 2 hours. The residue was purified by prep-HPLC (Column: Waters Xbridge 21.2×250 mm C18, 10 μm, Mobile Phase: A: water (10 mmol/L ammonium bicarbonate), B: acetonitrile) to afford N-(4-methyl-3-(7-(methylamino)-1,6-naphthyridin-3-yl)phenyl)-2-(3-(trifluoromethyl)-1H-1,2,4-triazol-1-yl)propanamide (100 mg, 0.22 mmol, 33%) as a yellow solid. MS (ESI) m/z 456.1 [M+H]⁺

Step 4. N-(4-Methyl-3-(7-(methylamino)-1,6-naphthyridin-3-yl)phenyl)-2-(3-(trifluoromethyl)-1H-1,2,4-triazol-1-yl)propanamide (100 mg, 0.22 mmol) was separated by chiral prep-HPLC (Instrument: SFC-150 (Waters); Column: OJ 20×250 mm, 10 μm (Daicel); Column temperature: 35° C.; Mobile phase: CO₂/MeOH (0.2% methanol ammonia)=70/30) to afford the first eluting enantiomer (retention time: 4.0 min) which was arbitrarily assigned as (S)—N-(4-methyl-3-(7-(methylamino)-1,6-naphthyridin-3-yl)phenyl)-2-(3-(trifluoromethyl)-1H-1,2,4-triazol-1-yl)propanamide (39.8 mg, 0.087 mmol) and the second eluting enantiomer (retention time: 5.2 min) which was arbitrarily assigned as (R)—N-(4-methyl-3-(7-(methylamino)-1,6-naphthyridin-3-yl)phenyl)-2-(3-(trifluoromethyl)-1H-1,2,4-triazol-1-yl)propanamide (44 mg, 0.097 mmol). ¹H NMR (400 MHz, DMSO-d₆) δ 10.53 (s, 1H), 9.06 (s, 1H), 8.98 (s, 1H), 8.78 (d, J=2.4 Hz, 1H), 8.22 (s, 1H), 7.61 (d, J=2.4 Hz, 1H), 7.52-7.50 (m, 1H), 7.33 (d, J=8.4 Hz, 1H), 6.93 (s, 1H), 6.62 (s, 1H), 5.42 (q, J=7.2 Hz, 1H), 2.87 (d, J=4.8 Hz, 3H), 2.26 (s, 3H), 1.85 (d, J=7.2 Hz, 3H). MS (ESI) m/z 456.1 [M+H]⁺

### Example 70. Synthesis of 3-Chloro-2-(2-cyanopropan-2-yl)-N-(6-methyl-5-(7-(methylamino)-1,6-naphthyridin-3-yl)pyridin-3-yl)isonicotinamide (Compound 70)

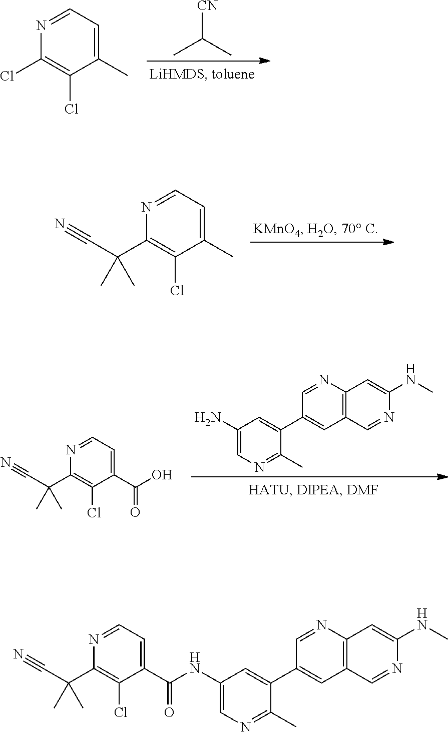

Step 1. To a solution of 2,3-dichloro-4-methylpyridine (300 mg, 1.85 mmol) and isobutyronitrile (127.8 mg, 1.85 mmol) in toluene (6 mL) was added lithium hexamethyldisilazide (1.6 M in tetrahydrofuran, 2.8 mL, 1.74 mmol) at 0° C. slowly. After stirring at 20° C. for 12 h, the reaction was quenched with saturated aqueous ammonium chloride solution (20 mL) at 0° C. and extracted with ethyl acetate (50 mL×3). The combined organic layers were washed with brine (50 mL), dried over anhydrous sodium sulfate, and filtered. The filtrate was concentrated under reduced pressure. The residue was purified by flash chromatography (silica, petroleum ether/ethyl acetate=1/10) to afford 2-(3-chloro-4-methylpyridin-2-yl)-2-methylpropanenitrile (285 mg, 1.47 mmol, 79%) as a colorless oil. MS (ESI) m/z 195.3[M+H]⁺

Step 2. A solution of 2-(3-chloro-4-methylpyridin-2-yl)-2-methylpropanenitrile (285 mg, 1.47 mmol), potassium permanganate (464.5 mg, 2.94 mmol) in water (10 mL) was stirred at 90° C. for 24 hours. After cooling to room temperature, the reaction mixture was adjusted to pH=3.0 with 2 N hydrochloric acid and extracted with ethyl acetate (50 mL). The organic layer was washed with water (25 mL) and brine (25 mL), dried over sodium sulfate, filtered and concentrated to afford 3-chloro-2-(2-cyanopropan-2-yl)isonicotinic acid (139 mg, 0.62 mmol, 42%) as a brown solid, which was used in the next step without further purification. MS (ESI) m/z 225.3 [M+H]⁺

Step 3. A mixture of 3-chloro-2-(2-cyanopropan-2-yl)isonicotinic acid (139 mg, 0.62 mmol), 3-(5-amino-2-methylpyridin-3-yl)-N-methyl-1,6-naphthyridin-7-amine (98.7 mg, 0.37 mmol), 1-[bis(dimethylamino)methylene]-1H-1,2,3-triazolo[4,5-b]pyridinium 3-oxid hexafluorophosphate (353.7 mg, 0.93 mmol) and N,N-diisopropylethylamine (240 mg, 1.86 mmol) in N,N-dimethylformamide (3 mL) was stirred at room temperature for 2 hours. The reaction mixture was concentrated. The residue was purified by prep-HPLC (Column: Sunfire Xbridge 4.6×50 mm C18, 3.5 μm, Mobile Phase: A: 0.01% aqueous ammonium bicarbonate, B: acetonitrile; B %: 5%-95% in 1.5 min) to afford 3-chloro-2-(2-cyanopropan-2-yl)-N-(6-methyl-5-(7-(methylamino)-1,6-naphthyridin-3-yl)pyridin-3-yl)isonicotinamide (2.3 mg, 0.0049 mmol, 0.8%) as a yellow solid. ¹H NMR (400 MHz, CDCl₃) δ 8.93-8.82 (m, 2H), 8.62 (d, J=4.6 Hz, 2H), 8.27 (d, J=2.6 Hz, 1H), 8.04 (d, J=7.8 Hz, 2H), 7.52 (d, J=4.8 Hz, 1H), 6.79 (s, 1H), 5.03 (s, 1H), 3.05 (d, J=5.1 Hz, 3H), 2.57 (s, 3H), 1.90 (s, 6H). MS (ESI) m/z 472.0 [M+H]⁺

### Example 71. Synthesis of 5-(2-hydroxyethoxy)-N-(4-methyl-3-(7-(methylamino)-1,6-naphthyridin-3-yl)phenyl)-4-(trifluoromethyl)picolinamide (Compound 71)

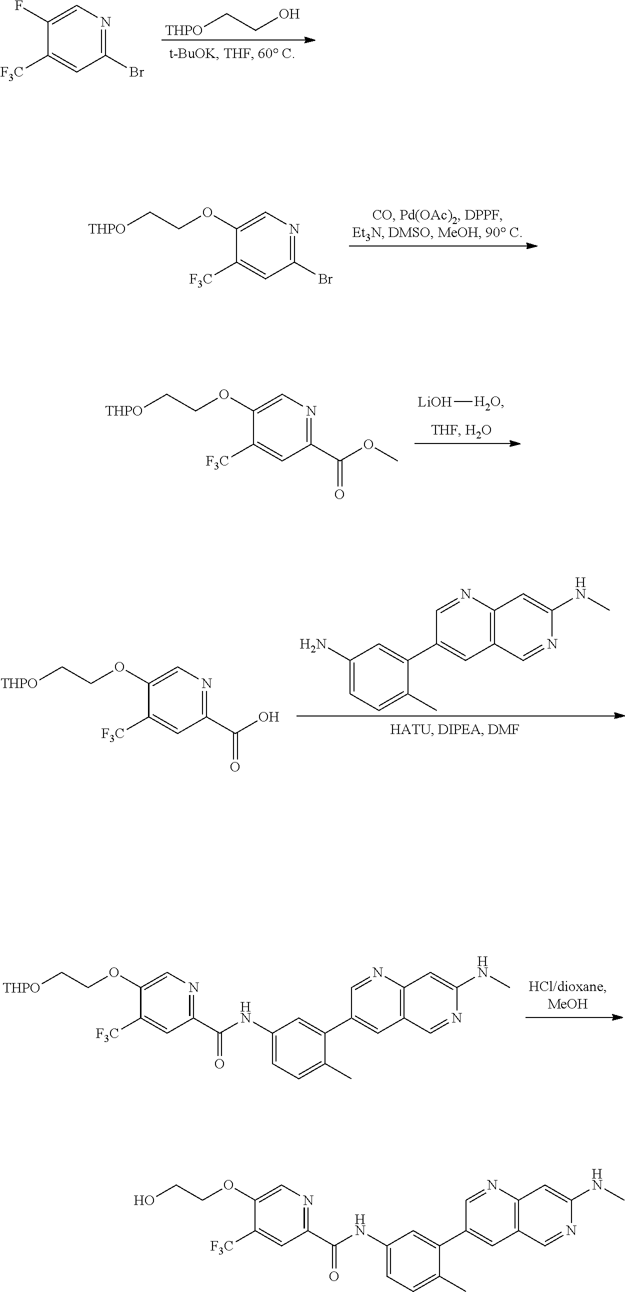

Step 1. A mixture of 2-bromo-5-fluoro-4-(trifluoromethyl)pyridine (300 mg, 1.2 mmol), 2-(tetrahydro-2H-pyran-2-yloxy)ethanol (193 mg, 1.32 mmol) and potassium 2-methylpropan-2-olate (1.32 mL, 1.32 mmol, 1 M in tetrahydrofuran) in tetrahydrofuran (10 mL) was stirred at 60° C. for 16 hours. The reaction mixture was concentrated. The residue was purified by flash chromatography (silica, ethyl acetate/petroleum ether=3/10) to afford 2-bromo-5-(2-(tetrahydro-2H-pyran-2-yloxy)ethoxy)-4-(trifluoromethyl)pyridine (250 mg, 0.68 mmol, 57%) as a white solid. MS (ESI) m/z 369.9 [M+H]⁺

Step 2. A mixture of 2-bromo-5-(2-(tetrahydro-2H-pyran-2-yloxy)ethoxy)-4-(trifluoromethyl)pyridine (200 mg, 0.54 mmol), 1,1′-bis(diphenylphosphino)ferrocene (120 mg, 0.216 mmol), palladium (II) acetate (24 mg, 0.108 mmol) and triethylamine (218 mg, 2.16 mmol) in dimethyl sulfoxide (12 mL) and methanol (8 mL) was stirred at 90° C. for 16 hours under carbon monoxide. The mixture was extracted with ethyl acetate (50 mL×2). The combined organic layer was washed with brine (30 mL×3), dried over anhydrous sodium sulfate, and filtered. The filtrate was concentrated under reduced pressure. The residue was purified by flash chromatography (silica, ethyl acetate/petroleum ether=10% to 30%) to afford methyl 5-(2-(tetrahydro-2H-pyran-2-yloxy)ethoxy)-4-(trifluoromethyl)picolinate (164 mg, 0.47 mmol, 87%) as a white solid. MS (ESI) m/z 350.0 [M+H]⁺

Step 3. To a solution of methyl 5-(2-(tetrahydro-2H-pyran-2-yloxy)ethoxy)-4-(trifluoromethyl)picolinate (205 mg, 0.59 mmol) in tetrahydrofuran (2 mL) were added lithium hydroxide hydrate (124 mg, 2.95 mmol) and water (2 mL), and the mixture was stirred at 25° C. for 16 hours. The solvent was evaporated under reduced pressure, and the residue was diluted with water (10 mL). The mixture was adjusted to pH 3 by adding 1 N hydrochloric acid slowly. The mixture was extracted with ethyl acetate (60 mL×2). The combined organic layers were dried over anhydrous sodium sulfate and filtered. The filtrate was concentrated under reduced pressure to afford 5-(2-(tetrahydro-2H-pyran-2-yloxy)ethoxy)-4-(trifluoromethyl)picolinic acid (145 mg, 0.43 mmol, 73%) as a white solid. MS (ESI) m/z 336.0 [M+H]⁺

Step 4. A mixture of 5-(2-((tetrahydro-2H-pyran-2-yl)oxy)ethoxy)-4-(trifluoromethyl)picolinic acid (20 mg, 0.06 mmol), 3-(5-amino-2-methylpyridin-3-yl)-N-methyl-1,6-naphthyridin-7-amine (9.5 mg, 0.036 mmol), 1-[bis(dimethylamino)methylene]-1H-1,2,3-triazolo[4,5-b]pyridinium 3-oxid hexafluorophosphate (34.2 mg, 0.09 mmol) and N,N-diisopropylethylamine (23.2 mg, 0.18 mmol) in N,N-dimethylformamide (3 mL) was stirred at room temperature for 2 hours. The reaction mixture was concentrated. The residue was diluted with ethyl acetate (50 mL), washed with water (25 mL) and brine (25 mL), dried over sodium sulfate, filtered, and concentrated. The residue was purified by flash chromatography (silica, dichloromethane/methanol=10/1) to afford N-(4-methyl-3-(7-(methylamino)-1,6-naphthyridin-3-yl)phenyl)-5-(2-((tetrahydro-2H-pyran-2-yl)oxy)ethoxy)-4-(trifluoromethyl)picolinamide (34 mg, 0.059 mmol, 98%) as a yellow solid. MS (EST) m/z 582.2 [M+H]⁺

Step 5. Hydrochloric acid (4 M in 1,4-dioxane, 3 mL) was added to a solution of N-(4-methyl-3-(7-(methylamino)-1,6-naphthyridin-3-yl)phenyl)-5-(2-((tetrahydro-2H-pyran-2-yl)oxy)ethoxy)-4-(trifluoromethyl)picolinamide (34 mg, 0.059 mmol) in methanol (1 mL) at room temperature. After stirring at room temperature for 6 h, the reaction mixture was concentrated. The residue was purified by prep-HPLC (Column: Sunfire Xbridge 4.6×50 mm C18, 3.5 μm, Mobile Phase: A: 0.01% aqueous ammonium bicarbonate, B: acetonitrile; B %: 5%-95% in 1.5 min) to afford 5-(2-hydroxyethoxy)-N-(4-methyl-3-(7-(methylamino)-1,6-naphthyridin-3-yl)phenyl)-4-(trifluoromethyl)picolinamide (9.2 mg, 0.019 mmol, 31%) as a yellow solid. ¹H NMR (400 MHz, CDCl₃) δ 9.74 (s, 1H), 8.96-8.79 (m, 2H), 8.46 (d, J=17.7 Hz, 2H), 8.03 (d, J=1.6 Hz, 1H), 7.76-7.66 (m, 2H), 7.35 (d, J=8.9 Hz, 2H), 6.79 (s, 1H), 4.98 (s, 1H), 4.54-4.34 (m, 2H), 4.07 (s, 2H), 3.04 (d, J=5.1 Hz, 3H), 2.33 (s, 3H). MS (ESI) m/z 498.1 [M+H]⁺

### Example 72. Synthesis of 5-(Difluoromethoxy)-N-(6-methyl-5-(7-(methylamino)-1,6-naphthyridin-3-yl)pyridin-3-yl)picolinamide (Compound 72)

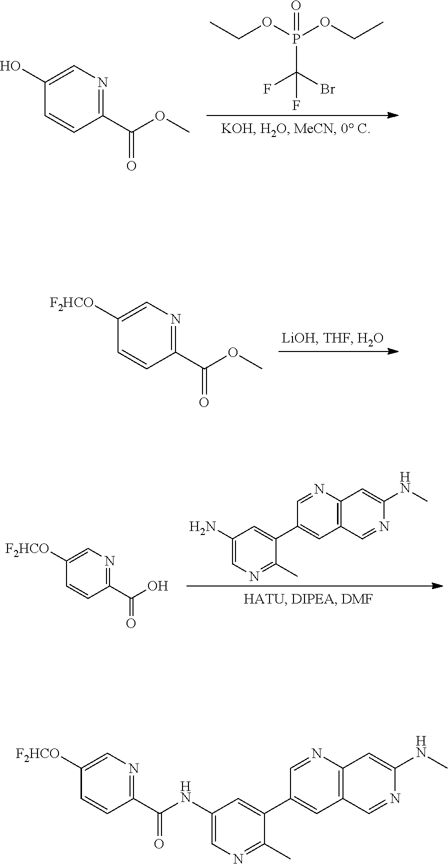 [SMILES: COC(=O)c1ccc(O)cn1 ; CCOP(=O)(OCC)C(F)(F)Br ; COC(=O)c1ccc(OC(F)F)cn1 ; O=C(O)c1ccc(OC(F)F)cn1 ; CNc1cc2ncc(-c3cc(N)cnc3C)cc2cn1 ; CNc1cc2ncc(-c3cc(NC(=O)c4ccc(OC(F)F)cn4)cnc3C)cc2cn1]

Step 1. Diethyl (bromodifluoromethyl)phosphonate (700 mg, 2.62 mmol) was added slowly to a solution of methyl 5-hydroxypicolinate (200 mg, 1.31 mmol) and potassium hydroxide (292 mg, 5.22 mmol) in acetonitrile (5 mL) and water (5 mL) at 0° C. over 5 min. After stirring at 0° C. for 1 h, the resulting solution was concentrated in vacuo and treated with ethyl acetate (20 mL). The organic phase was washed with brine (20 mL), dried over anhydrous sodium sulfate, and filtered. The filtrate was concentrated in vacuo and purified by flash chromatography (silica, ethyl acetate/petroleum ether=0% to 50%) to afford methyl 5-(difluoromethoxy)picolinate (64 mg, 0.32 mmol, 24%) as a white solid. MS (ESI) m/z 204.1 [M+H]⁺

Step 2. A mixture of methyl 5-(difluoromethoxy)picolinate (70 mg, 0.34 mmol) in tetrahydrofuran (2 mL) and water (2 mL) was added lithium hydroxide monohydrate (55 mg, 1.32 mmol) at room temperature. After stirring at room temperature for 1 h, the resulting mixture was concentrated under reduced pressure and treated with aqueous hydrochloric acid solution (1M, 5 mL). The aqueous phase was extracted with ethyl acetate (10 mL×3). The combined organic phases were dried over anhydrous sodium sulfate and filtered. The filtrate was concentrated in vacuo to afford 5-(difluoromethoxy)picolinic acid (60 mg, 0.32 mmol, 94%) as a white solid. MS (ESI) m/z 190.1 [M+H]⁺

Step 3. To a solution of 5-(difluoromethoxy)picolinic acid (60 mg, 0.32 mmol) in N,N-dimethylformamide (2 mL) was added N,N-diisopropylethylamine (62 mg, 0.48 mmol) and 1-[bis(dimethylamino)methylene]-1H-1,2,3-triazolo[4,5-b]pyridinium 3-oxid hexafluorophosphate (146 mg, 0.38 mmol). The solution was stirred at room temperature for 5 min, then 3-(5-amino-2-methylpyridin-3-yl)-N-methyl-1,6-naphthyridin-7-amine (85 mg, 0.32 mmol) was added. After stirring at room temperature for 16 h, the resulting solution was poured into water (20 mL) and extracted with ethyl acetate (20 mL×3). The combined organic phases were washed with brine (20 mL), dried over sodium sulfate, filtered, and concentrated. The residue was purified by flash chromatography (silica, methanol/dichloromethane=0% to 10%) to afford the crude solid. The solid was treated with methanol (2 mL) and stirred for 5 min. The mixture was filtered and the filter cake was dried under reduced pressure to afford 5-(difluoromethoxy)-N-(6-methyl-5-(7-(methylamino)-1,6-naphthyridin-3-yl)pyridin-3-yl)picolinamide (90.0 mg, 0.21 mmol, 64%) as a yellow solid. ¹H NMR (400 MHz, DMSO-d₆) δ 10.94 (s, 1H), 9.03 (d, J=2.4 Hz, 1H), 9.00 (s, 1H), 8.87 (d, J=2.4 Hz, 1H), 8.64 (d, J=2.4 Hz, 1H), 8.33-8.30 (m, 2H), 8.25 (d, J=8.4 Hz, 1H), 7.94-7.94 (m, 1H), 7.51 (t, J=73.0 Hz, 1H), 6.99-6.96 (m, 1H), 6.64 (s, 1H), 2.88 (d, J=4.8 Hz, 3H), 2.49 (s, 3H). MS (ESI) m/z 436.6 [M+H]⁺

### Example 73. Synthesis of N-(6-methyl-5-(7-(methylamino)-1,6-naphthyridin-3-yl)pyridazin-3-yl)-4-(trifluoromethyl)picolinamide (Compound 75)

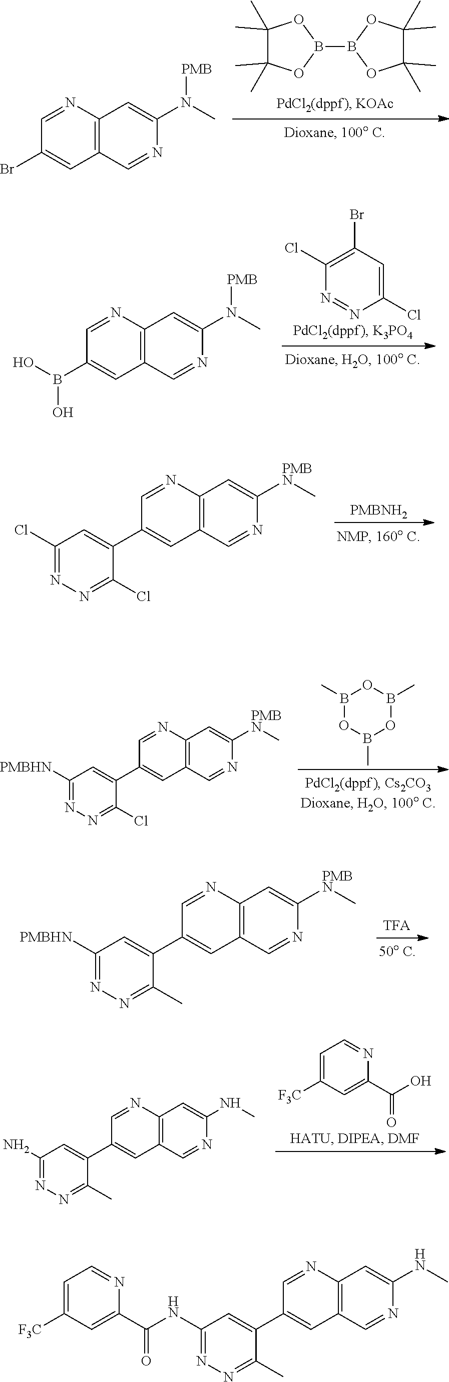

Step 1. A mixture of 3-bromo-N-(4-methoxybenzyl)-N-methyl-1,6-naphthyridin-7-amine (3.57 g, 10.0 mmol), 4,4,4′,4′,5,5,5′,5′-octamethyl-2,2′-bi(1,3,2-dioxaborolane) (3.04 g, 12.0 mmol), 1,1′-bis(diphenylphosphino)ferrocene-palladium(II)dichloride dichloromethane complex (816 mg, 1.0 mmol) and potassium acetate (2.94 g, 30.0 mmol) in dioxane (30 mL) was stirred at 100° C. for 1 hour under nitrogen. After cooling to room temperature, the reaction mixture was used in the next step directly. MS (ESI) m/z 324.1 [M+H]⁺

Step 2. To the reaction solution in the previous step were added 4-bromo-3,6-dichloropyridazine (2.28 g, 10 mmol), 1,1′-bis(diphenylphosphino)ferrocene-palladium(II)dichloride dichloromethane complex (816 mg, 1.0 mmol), potassium phosphate tribasic (6.0 g, 28.3 mmol) and water (15 mL). The reaction mixture was stirred at 100° C. for 2 hours under nitrogen. The reaction was cooled to room temperature and poured into water (40 mL), and then extracted with ethyl acetate (50 mL×3). The organic layers were concentrated and purified by flash chromatography (silica, dichloromethane/methanol=10/1) to give 3-(3,6-dichloropyridazin-4-yl)-N-(4-methoxybenzyl)-N-methyl-1,6-naphthyridin-7-amine (2.0 g, 4.7 mmol, 47%) as a yellow solid. MS (ESI) m/z 426.1 [M+H]⁺

Step 3. A mixture of 3-(3,6-dichloropyridazin-4-yl)-N-(4-methoxybenzyl)-N-methyl-1,6-naphthyridin-7-amine (480 mg, 1.13 mmol) and (4-methoxyphenyl)methanamine (200 mg, 1.46 mmol) in 1-methyl-2-pyrrolidinone (2 mL) was stirred at 160° C. for 6 hours. The reaction was cooled to room temperature, poured into water (20 mL) and extracted with ethyl acetate (20 mL×3). The organic layers were concentrated and purified by flash chromatography (silica, dichloromethane/methanol=10/1) to give 3-(3-chloro-6-((4-methoxybenzyl)amino)pyridazin-4-yl)-N-(4-methoxybenzyl)-N-methyl-1,6-naphthyridin-7-amine (230 mg, 0.44 mmol, 38%) as a yellow solid. MS (EST) m/z 527.2 [M+H]⁺

Step 4. A mixture of 3-(3-chloro-6-((4-methoxybenzyl)amino)pyridazin-4-yl)-N-(4-methoxybenzyl)-N-methyl-1,6-naphthyridin-7-amine (230 mg, 0.44 mmol), 2,4,6-trimethyl-1,3,5,2,4,6-trioxatriborinane (111 mg, 0.88 mmol), 1,1′-bis(diphenylphosphino)ferrocene-palladium(II)dichloride dichloromethane complex (31 mg, 0.038 mmol) and cesium carbonate (430 mg, 1.32 mmol) in dioxane (5 mL) and water (2 mL) was stirred at 100° C. for 2 hours under nitrogen. After cooling to room temperature, the reaction mixture was poured into water (10 mL) and extracted with ethyl acetate (20 mL×3). The combined organic phases were concentrated and purified by flash chromatography (silica, dichloromethane/methanol=10/2) to give N-(4-methoxybenzyl)-3-(6-((4-methoxybenzyl)amino)-3-methylpyridazin-4-yl)-N-methyl-1,6-naphthyridin-7-amine (80 mg, 0.158 mmol, 36%) as a yellow solid. MS (ESI) m/z 507.1 [M+H]⁺

Step 5. A solution of N-(4-methoxybenzyl)-3-(6-((4-methoxybenzyl)amino)-3-methylpyridazin-4-yl)-N-methyl-1,6-naphthyridin-7-amine (80 mg, 0.158 mmol) in trifluoroacetic acid (10 mL) was stirred at 50° C. for 2 hours. The mixture was concentrated and purified by flash chromatography (silica, dichloromethane/methanol=20/3) to give 3-(6-amino-3-methylpyridazin-4-yl)-N-methyl-1,6-naphthyridin-7-amine (30 mg, 0.113 mmol, 75%) as a yellow solid. MS (ESI) m/z 266.1 [M+H]⁺

Step 6. A mixture of 3-(6-amino-3-methylpyridazin-4-yl)-N-methyl-1,6-naphthyridin-7-amine (120 mg, 0.45 mmol), 4-(trifluoromethyl)picolinic acid (86 mg, 0.45 mmol), 1-[bis(dimethylamino)methylene]-1H-1,2,3-triazolo[4,5-b]pyridinium 3-oxid hexafluorophosphate (HATU, 513 mg, 1.35 mmol) and N,N-diisopropylethylamine (174 mg, 1.35 mmol) in N,N-dimethylformamide (10 mL) was stirred at 25° C. for 1 hour. The reaction was diluted with water (20 mL) and extracted with ethyl acetate (30 mL×2). The combined organic phases were washed with brine (30 mL), dried over sodium sulfate, filtered, and concentrated. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(6-methyl-5-(7-(methylamino)-1,6-naphthyridin-3-yl)pyridazin-3-yl)-4-(trifluoromethyl)picolinamide (13.7 mg, 0.03 mmol, 7%) as a yellow solid. ¹H NMR (400 MHz, DMSO-d₆) δ 11.07 (s, 1H), 9.10 (d, J=5.1 Hz, 1H), 9.03 (s, 1H), 8.95 (d, J=2.3 Hz, 1H), 8.50 (d, J=1.9 Hz, 1H), 8.43 (s, 1H), 8.39 (s, 1H), 8.18 (d, J=4.7 Hz, 1H), 7.10 (d, J=4.3 Hz, 1H), 6.65 (s, 1H), 2.89 (d, J=4.1 Hz, 3H), 2.70 (s, 3H). MS (EST) m/z 440.1 [M+H]⁺

### Example 74. Synthesis of 4-(2-cyanopropan-2-yl)-N-(6-methyl-5-(7-(methylamino)-1,6-naphthyridin-3-yl)pyridazin-3-yl)picolinamide (Compound 77)

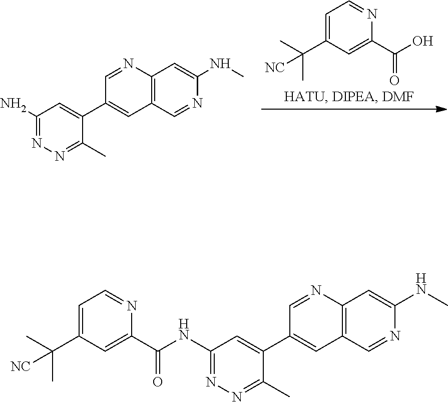

Step 1. A mixture of 3-(6-amino-3-methylpyridazin-4-yl)-N-methyl-1,6-naphthyridin-7-amine (30 mg, 0.113 mmol), 4-(2-cyanopropan-2-yl)picolinic acid (21.4 mg, 0.113 mmol), 1-[bis(dimethylamino)methylene]-1H-1,2,3-triazolo[4,5-b]pyridinium 3-oxid hexafluorophosphate (64 mg, 0.17 mmol) and N,N-diisopropylethylamine (44 mg, 0.34 mmol) in N,N-dimethylformamide (10 mL) was stirred at 25° C. for 1 hour. The reaction was diluted with water (20 mL) and extracted with ethyl acetate (30 mL×2). The combined organic phases were washed with brine (30 mL), dried over sodium sulfate, filtered, and concentrated. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(2-cyanopropan-2-yl)-N-(6-methyl-5-(7-(methylamino)-1,6-naphthyridin-3-yl)pyridazin-3-yl)picolinamide (17.0 mg, 0.039 mmol, 34%) as a yellow solid. ¹H NMR (400 MHz, DMSO-d₆) δ 10.99 (s, 1H), 9.04 (s, 1H), 8.96 (d, J=2.4 Hz, 1H), 8.87 (d, J=5.7 Hz, 1H), 8.51 (d, J=1.9 Hz, 1H), 8.46 (s, 1H), 8.31 (d, J=1.6 Hz, 1H), 7.93 (dd, J=5.2, 2.0 Hz, 1H), 7.11 (s, 1H), 6.66 (s, 1H), 2.89 (s, 3H), 2.70 (s, 3H), 1.78 (s, 6H). MS (ESI) m/z 439.2 [M+H]⁺

### Example 75. Synthesis of N-(3-(7-((2-cyanoethyl)amino)-1,6-naphthyridin-3-yl)-4-methylphenyl)-4-(difluoromethyl)picolinamide (Compound 78)

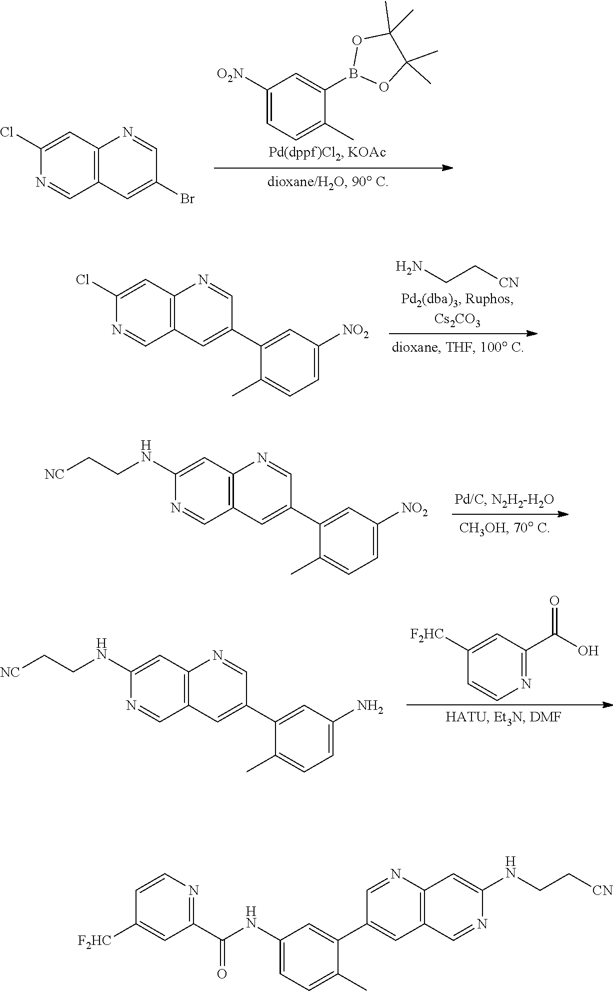

Step 1. A solution of 3-bromo-7-chloro-1,6-naphthyridine (300 mg, 1.23 mmol), 4,4,5,5-tetramethyl-2-(2-methyl-5-nitrophenyl)-1,3,2-dioxaborolane (323 mg, 1.23 mmol), 1,1′-bis(diphenylphosphino) ferrocene-palladium(II) dichloride dichloromethane complex (98 mg, 0.12 mmol) and potassium acetate (509 mg, 3.69 mmol) in 1,4-dioxane (10 mL) and water (1 mL) was stirred at 90° C. under a nitrogen atmosphere for 3 hours. The mixture was cooled to room temperature and concentrated. The crude residue was purified flash chromatography (silica, petroleum ether/ethyl acetate=3/1) to give 7-chloro-3-(2-methyl-5-nitrophenyl)-1,6-naphthyridine (246 mg, 0.82 mmol, 67%) as a yellow oil. MS (ESI) m/z 300.0 [M+H]⁺

Step 2. A solution of 7-chloro-3-(2-methyl-5-nitrophenyl)-1,6-naphthyridine (246 mg, 0.82 mmol), 3-aminopropanenitrile (58 mg, 0.82 mmol), tris(dibenzylideneacetone)dipalladium (73 mg, 0.08 mmol), 2-dicyclohexylphosphino-2′,6′-diisopropoxy-1,1′-biphenyl (RuPhos, 186 mg, 0.4 mmol) and cesium carbonate (935 mg, 2.46 mmol) in 1,4-dioxane (10 mL) and tetrahydrofuran (10 mL) was stirred at 100° C. for 1 hour. The mixture was cooled to room temperature and concentrated. The crude residue was purified flash chromatography (silica, petroleum ether/ethyl acetate=1/1) to give 3-((3-(2-methyl-5-nitrophenyl)-1,6-naphthyridin-7-yl)amino)propanenitrile (66 mg, 0.20 mmol, 24%) as a yellow oil. MS (ESI) m/z 333.8 [M+H]⁺

Step 3. A solution of 3-((3-(2-methyl-5-nitrophenyl)-1,6-naphthyridin-7-yl)amino)propanenitrile (66 mg, 0.20 mmol) in methanol (10 mL) was added hydrazine hydrate (5 mg, 0.1 mmol) and 10% palladium on carbon (7 mg). The mixture was stirred at 70° C. for 1 hour. After cooling to room temperature, the reaction mixture was filtered through celite. The filtrate was concentrated. The residue was purified by flash chromatography (silica, petroleum ether/ethyl acetate=1/2) to give 3-((3-(5-amino-2-methylphenyl)-1,6-naphthyridin-7-yl)amino)propanenitrile (48 mg, 0.16 mmol, 79%) as a yellow oil. MS (ESI) m/z 304.0 [M+H]⁺

Step 4. A solution 4-(difluoromethyl)picolinic acid (27 mg, 0.16 mmol), 3-((3-(5-amino-2-methylphenyl)-1,6-naphthyridin-7-yl)amino)propanenitrile (48 mg, 0.16 mmol), 1-[bis(dimethylamino)methylene]-1H-1,2,3-triazolo[4,5-b]pyridinium 3-oxid hexafluorophosphate (182 mg, 0.48 mmol) and triethylamine (0.2 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%/6-70% in 15 min) to afford N-(3-(7-((2-cyanoethyl)amino)-1,6-naphthyridin-3-yl)-4-methylphenyl)-4-(difluoromethyl)picolinamide (6.1 mg, 0.013 mmol, 8%) as a yellow solid. ¹H NMR (400 MHz, DMSO-d₆) δ 10.81 (s, 1H), 9.04 (s, 1H), 8.93 (d, J=4.9 Hz, 1H), 8.86 (d, J=2.3 Hz, 1H), 8.31-8.27 (m, 2H), 7.91 (ddd, J=11.9, 10.2, 3.5 Hz, 3H), 7.30 (dt, J=71.0, 30.8 Hz, 3H), 6.87 (s, 1H), 3.64 (q, J=6.4 Hz, 2H), 2.84 (t, J=6.5 Hz, 2H), 2.30 (s, 3H). MS (ESI) m/z 459.1 [M+H]⁺

### Example 76. Synthesis of N-(3-(2-cyano-7-(methylamino)-1,6-naphthyridin-3-yl)-4-methylphenyl)-2-(trifluoromethyl)isonicotinamide (Compound 79)

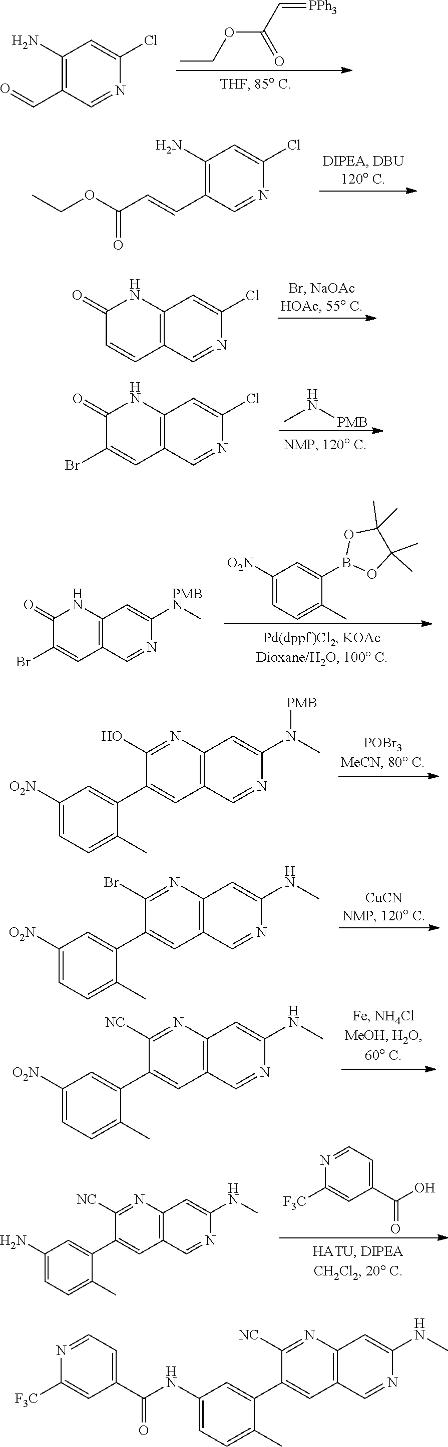

Step 1. A solution of 4-amino-6-chloronicotinaldehyde (3.12 g, 20 mmol) and ethyl 2-(triphenyl-15-phosphaneylidene)acetate (8.35 g, 24 mmol) in tetrahydrofuran (40 mL) was stirred at 85° C. for 16 hours. The reaction was poured into water (40 mL) and extracted with ethyl acetate (50 mL×3). The organic phases were concentrated and purified by flash chromatography (silica, petroleum ether/ethyl acetate=1/2) to give ethyl (E)-3-(4-amino-6-chloropyridin-3-yl)acrylate (4.4 g, 19.4 mmol, 97%) as a yellow solid. MS (ESI) m/z 227.1 [M+H]⁺

Step 2. A solution of ethyl (E)-3-(4-amino-6-chloropyridin-3-yl)acrylate (4.4 g, 19.4 mmol) and 1,8-diazabicyclo[5.4.0]undec-7-ene (DBU, 4.9 g, 31 mmol) in N,N-diisopropylethylamine (20 mL) was stirred at 120° C. for 16 hours. The reaction was poured into water (20 mL) and filtered. The filter cake was washed with ethyl acetate (40 mL) and dried to obtain 7-chloro-1,6-naphthyridin-2(1H)-one (1.44 g, 8 mmol, 41%) as a yellow solid. MS (ESI) m/z 181.1 [M+H]⁺

Step 3. To a solution of 7-chloro-1,6-naphthyridin-2(1H)-one (1.44 g, 8 mmol) and sodium acetate (1.31 g, 16 mmol) in acetic acid (20 mL) was added bromine (2.3 mL, 4.2 mmol) in four portions over four days at 60° C. The reaction was poured into water (20 mL) and filtered. The filter cake was washed with water (40 mL) and dried to give 3-bromo-7-chloro-1,6-naphthyridin-2(1H)-one (1.5 g, 5.7 mmol, 71%) as a white solid. MS (EST) m/z 258.8 [M+H]⁺

Step 4. A solution of 3-bromo-7-chloro-1,6-naphthyridin-2(1H)-one (1.5 g, 5.7 mmol) and 1-(4-methoxyphenyl)-N-methylmethanamine (2.6 g, 17.3 mmol) in 1-methyl-2-pyrrolidinone (20 mL) was stirred at 120° C. for 16 hours. The mixture was poured into water (20 mL) and filtered. The filter cake was washed with ethyl acetate (40 mL) and dried to give 3-bromo-7-((4-methoxybenzyl)methyl)amino)-1,6-naphthyridin-2(1H)-one (850 mg, 2.2 mmol, 40%) as a yellow solid. MS (ESI) m/z 373.6 [M+H]⁺

Step 5. A mixture of 3-bromo-7-((4-methoxybenzyl)(methyl)amino)-1,6-naphthyridin-2(1H)-one (1.4 g, 3.74 mmol), 4,4,5,5-tetramethyl-2-(2-methyl-5-nitrophenyl)-1,3,2-dioxaborolane (1.18 g, 4.49 mmol), 1,1′-bis(diphenylphosphino)ferrocene-palladium(II)dichloride dichloromethane complex (305 mg, 0.374 mmol) and potassium acetate (1.55 g, 11.22 mmol) in dioxane (15 mL) and water (5 mL) was stirred at 100° C. for 1 hour under nitrogen. After cooling to room temperature, the reaction mixture was poured into water (30 mL) and extracted with ethyl acetate (30 mL×3). The combined organic phases were concentrated and purified by flash chromatography (silica, dichloromethane/ethyl acetate=1/1) to give 7-((4-methoxybenzyl)(methyl)amino)-3-(2-methyl-5-nitrophenyl)-1,6-naphthyridin-2-ol (980 mg, 2.28 mmol, 61%) as a yellow solid. MS (ESI) m/z 431.1[M+H]⁺

Step 6. A mixture of 7-((4-methoxybenzyl)(methyl)amino)-3-(2-methyl-5-nitrophenyl)-1,6-naphthyridin-2-ol (260 mg, 0.60 mmol) and phosphoryl tribromide (0.5 g) in acetonitrile (10 mL) was stirred at 80° C. for 3 hours under nitrogen. After cooling to room temperature, the reaction mixture was poured into ice water (20 mL) and extracted with ethyl acetate (20 mL×2). The organic phases were concentrated and purified by flash chromatography (silica, methanol/dichloromethane=1/10) to give 2-bromo-N-methyl-3-(2-methyl-5-nitrophenyl)-1,6-naphthyridin-7-amine (90 mg, 0.241 mmol, 40%) as a yellow solid. MS (ESI) m/z 373.0[M+H]⁺

Step 7. A mixture of 2-bromo-N-methyl-3-(2-methyl-5-nitrophenyl)-1,6-naphthyridin-7-amine (90 mg, 0.241 mmol) and cyanocopper (64 mg, 0.72 mmol) in 1-methyl-2-pyrrolidinone (1 mL) was stirred at 120° C. for 3 hours under nitrogen. After cooling to room temperature, the reaction mixture was poured into water (10 mL) and extracted with ethyl acetate (20 mL×3). The combined organic phases were concentrated and purified by flash chromatography (silica, methanol/dichloromethane=1/10) to give 3-(2-methyl-5-nitrophenyl)-7-(methylamino)-1,6-naphthyridine-2-carbonitrile (30 mg, 0.094 mmol, 39%) as a yellow solid. MS (ESI) m/z 320.1 [M+H]⁺

Step 8. A mixture of 3-(2-methyl-5-nitrophenyl)-7-(methylamino)-1,6-naphthyridine-2-carbonitrile (30 mg, 0.094 mmol), iron (0.1 g, 1.84 mmol) and ammonium chloride (48 mg, 0.92 mmol) in methanol (6 mL) and water (6 mL) was stirred at 60° C. for 1 hour. The reaction was filtered and washed with methanol (30 mL). The filtrate was concentrated to give 3-(5-amino-2-methylphenyl)-7-(methylamino)-1,6-naphthyridine-2-carbonitrile (20 mg, 0.069 mmol, 74%) as a yellow solid. MS (ESI) m/z 290.1 [M+H]⁺

Step 9. A mixture of 3-(5-amino-2-methylphenyl)-7-(methylamino)-1,6-naphthyridine-2-carbonitrile (20 mg, 0.069 mmol), 2-(trifluoromethyl)isonicotinic acid (14 mg, 0.07 mmol), 1-[bis(dimethylamino)methylene]-1H-1,2,3-triazolo[4,5-b]pyridinium 3-oxid hexafluorophosphate (80 mg, 0.21 mmol) and N,N-diisopropylethylamine (27 mg, 0.21 mmol) in dichloromethane (10 mL) was stirred at room temperature for 30 minutes. The mixture was poured into water (20 mL) and extracted with dichloromethane (20 mL×3). The organic phases were concentrated and purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(3-(2-cyano-7-(methylamino)-1,6-naphthyridin-3-yl)-4-methylphenyl)-2-(trifluoromethyl)isonicotinamide (2.3 mg, 0.005 mmol, 7%) as a yellow solid. ¹H NMR (400 MHz, DMSO-d₆) δ 10.78 (s, 1H), 9.13 (s, 1H), 8.99 (d, J=5.0 Hz, 1H), 8.46 (s, 1H), 8.37 (s, 1H), 8.20 (d, J=5.9 Hz, 1H), 7.87-7.75 (m, 2H), 7.45 (d, J=8.4 Hz, 1H), 7.31 (s, 1H), 6.65 (s, 1H), 2.89 (d, J=3.6 Hz, 3H), 2.18 (s, 3H). MS (ESI) m/z 463.0 [M+H]⁺

### Example 77. Synthesis of (R)—N-(4-methyl-3-(7-(methylamino)-1,6-naphthyridin-3-yl)phenyl)-4-(2,2,2-trifluoro-1-hydroxyethyl)picolinamide and (S)—N-(4-methyl-3-(7-(methylamino)-1,6-naphthyridin-3-yl)phenyl)-4-(2,2,2-trifluoro-1-hydroxyethyl)picolinamide (Compounds 80 and 81)

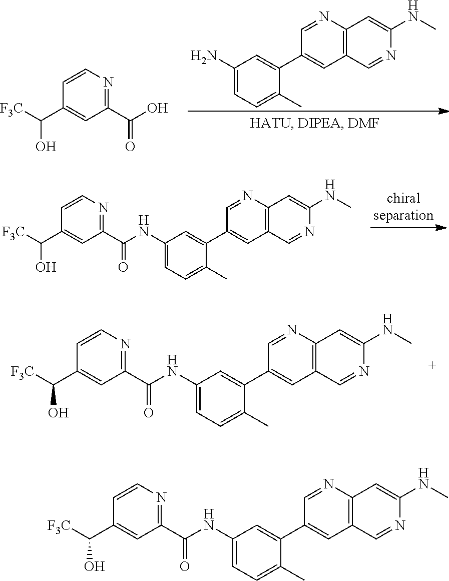 [SMILES: O=C(O)c1cc(C(O)C(F)(F)F)ccn1 ; CNc1cc2ncc(-c3cc(N)ccc3C)cc2cn1 ; CNc1cc2ncc(-c3cc(NC(=O)c4cc(C(O)C(F)(F)F)ccn4)ccc3C)cc2cn1 ; CNc1cc2ncc(-c3cc(NC(=O)c4cc([C@@H](O)C(F)(F)F)ccn4)ccc3C)cc2cn1 ; CNc1cc2ncc(-c3cc(NC(=O)c4cc([C@H](O)C(F)(F)F)ccn4)ccc3C)cc2cn1]

Step 1. A solution of 4-(2,2,2-trifluoro-1-hydroxyethyl)picolinic acid (50 mg, 0.23 mmol), 3-(5-amino-2-methylphenyl)-N-methyl-1,6-naphthyridin-7-amine (35.8 mg, 0.14 mmol), 1-[bis(dimethylamino)methylene]-1H-1,2,3-triazolo[4,5-b]pyridinium 3-oxid hexafluorophosphate (129 mg, 0.34 mmol) and N,N-diisopropylethylamine (87.6 mg, 0.68 mmol) in N,N-dimethylformamide (3 mL) was stirred at room temperature for 2 hours. The reaction solution was concentrated. The residue was purified by prep-HPLC (Column: Xbridge 21.2×250 mm C18, 10 μm, Mobile Phase A: water (10 mM ammonium bicarbonate), B: acetonitrile) to give N-(4-methyl-3-(7-(methylamino)-1,6-naphthyridin-3-yl)phenyl)-4-(2,2,2-trifluoro-1-hydroxyethyl)picolinamide (52 mg, 0.11 mmol, 48%) as a yellow solid. MS (ESI) m/z 468.1 [M+H]⁺

Step 2. The enantiomers of N-(4-methyl-3-(7-(methylamino)-1,6-naphthyridin-3-yl)phenyl)-4-(2,2,2-trifluoro-1-hydroxyethyl)picolinamide (52 mg, 0.11 mmol) were separated by chiral SFC (Instrument: SFC-150 (Waters); Column: OJ 20×250 mm, 10 μm (Daicel); Mobile phase: CO₂/MeOH (0.2% methanol ammonia)=70/30; Detection wavelength: 214 nm) to afford the first eluting enantiomer which was arbitrarily assigned as (R)—N-(4-methyl-3-(7-(methylamino)-1,6-naphthyridin-3-yl)phenyl)-4-(2,2,2-trifluoro-1-hydroxyethyl)picolinamide (15.6 mg, 0.033 mmol). ¹H NMR (400 MHz, DMSO-d₆) δ 10.73 (s, 1H), 8.98 (s, 1H), 8.81 (dd, J=11.6, 3.6 Hz, 2H), 8.32-8.21 (m, 2H), 7.97-7.86 (m, 2H), 7.79 (d, J=5.4 Hz, 1H), 7.36 (d, J=8.4 Hz, 1H), 7.28 (d, J=5.8 Hz, 1H), 6.89 (d, J=5.0 Hz, 1H), 6.63 (s, 1H), 5.61-5.45 (m, 1H), 2.87 (d, J=5.0 Hz, 3H), 2.30 (s, 3H). MS (ESI) m/z 468.1 [M+H]˜; and the second eluting enantiomer which was arbitrarily assigned as (S)—N-(4-methyl-3-(7-(methylamino)-1,6-naphthyridin-3-yl)phenyl)-4-(2,2,2-trifluoro-1-hydroxyethyl)picolinamide (13.2 mg, 0.028 mmol). ¹H NMR (400 MHz, DMSO-d₆) δ 10.73 (s, 1H), 8.98 (s, 1H), 8.81 (dd, J=11.8, 3.7 Hz, 2H), 8.36-8.21 (m, 2H), 7.97-7.85 (m, 2H), 7.79 (d, J=4.6 Hz, 1H), 7.36 (d, J=8.4 Hz, 1H), 7.28 (d, J=5.8 Hz, 1H), 6.89 (d, J=5.0 Hz, 1H), 6.63 (s, 1H), 5.58-5.41 (m, 1H), 2.87 (d, J=5.0 Hz, 3H), 2.30 (s, 3H). MS (ESI) m/z 468.1 [M+H]⁺

### Example 78. Synthesis of 5-(difluoromethoxy)-4-methyl-N-(6-methyl-5-(7-(methylamino)-1,6-naphthyridin-3-yl)pyridin-3-yl)picolinamide (Compound 82)

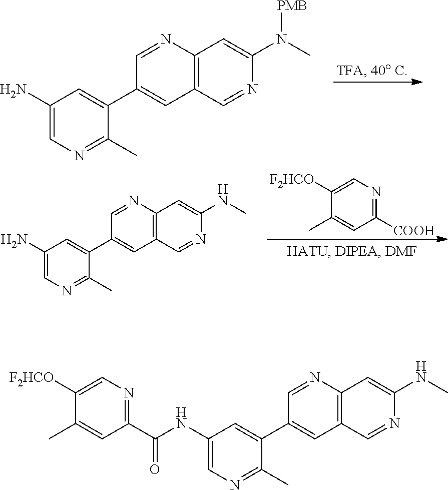

Step 1. A mixture of 3-(5-amino-2-methylpyridin-3-yl)-N-(4-methoxybenzyl)-N-methyl-1,6-naphthyridin-7-amine (6.17 g, 16.0 mmol, crude), and trifluoroacetic acid (40 mL) was stirred at 40° C. for 1 hour. The reaction was concentrated in vacuo and the residue was adjusted to pH=3-4 with ammonia (7 N in methanol). The residue was purified by flash chromatography (silica, methanol/dichloromethane=0-10%) to give 3-(5-amino-2-methylpyridin-3-yl)-N-methyl-1,6-naphthyridin-7-amine (3.1 g, 11.7 mmol, 73%) as a yellow solid. MS (ESI) m/z 266.1 [M+H]⁺

Step 2. A solution of 3-(5-amino-2-methylpyridin-3-yl)-N-methyl-1,6-naphthyridin-7-amine (30 mg, 0.11 mmol), 5-(difluoromethoxy)-4-methylpicolinic acid (31 mg, 0.15 mmol), 1-[bis(dimethylamino)methylene]-1H-1,2,3-triazolo[4,5-b]pyridinium 3-oxid hexafluorophosphate (114 mg, 0.3 mmol) and N,N-diisopropylethylamine (58.0 mg, 0.45 mmol) in N,N-dimethylformamide (5 mL) was stirred at room temperature for 2 hours. The reaction was poured into water (20 mL) and extracted with ethyl acetate (20 mL×3). The combined organic phases were concentrated and purified by prep-HPLC (Column: Welch Xtimate 21.2×250 mm C18, 10 μm, Mobile Phase: A: water (10 mM ammonium bicarbonate), B: acetonitrile; B %: 30%-70% in min) to give 5-(difluoromethoxy)-4-methyl-N-(6-methyl-5-(7-(methylamino)-1,6-naphthyridin-3-yl)pyridin-3-yl)picolinamide (11.4 mg, 0.025 mmol) as a yellow solid. ¹H NMR (500 MHz, DMSO-d₆) δ 10.91 (s, 1H), 9.03 (d, J=2.3 Hz, 1H), 8.99 (s, 1H), 8.86 (d, J=2.3 Hz, 1H), 8.52 (s, 1H), 8.32 (d, J=2.0 Hz, 1H), 8.29 (d, J=2.3 Hz, 1H), 8.16 (s, 1H), 7.46 (t, J=73.1 Hz, 1H), 6.96 (q, J=4.8 Hz, 1H), 6.64 (s, 1H), 2.88 (t, J=4.8 Hz, 3H), 2.49 (s, 3H), 2.39 (s, 3H). MS (ESI) m/z 451.1 [M+H]⁺

### Example 79. Synthesis of 3-chloro-N-(6-methyl-5-(7-(methylamino)-1,6-naphthyridin-3-yl)pyridin-3-yl)-2-(trifluoromethyl)isonicotinamide (Compound 83)

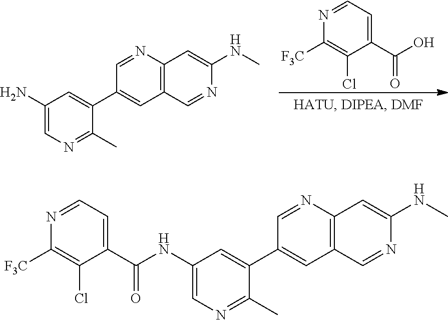

A solution of 3-chloro-2-(trifluoromethyl)isonicotinic acid (25 mg, 0.11 mmol), 3-(5-amino-2-methylpyridin-3-yl)-N-methyl-1,6-naphthyridin-7-amine (31 mg, 0.11 mmol), 1-[bis(dimethylamino)methylene]-1H-1,2,3-triazolo[4,5-b]pyridinium 3-oxid hexafluorophosphate (70 mg, 0.16 mmol), and N,N-diisopropylethylamine (0.2 mL) in N,N-dimethylformamide (2 mL) was stirred at room temperature for 2 hours. The reaction mixture was concentrated and the residue purified by prep-HPLC (Column: Xbridge 21.2×250 mm C18, 10 μm, Mobile Phase A: water (10 mM ammonium bicarbonate), B: acetonitrile) to give 3-chloro-N-(6-methyl-5-(7-(methylamino)-1,6-naphthyridin-3-yl)pyridin-3-yl)-2-(trifluoromethyl)isonicotinamide (19 mg, 0.040 mmol, 40%) as a yellow solid. ¹H NMR (400 MHz, DMSO-d₆) δ 11.14 (s, 1H), 9.00 (s, 1H), 8.86 (dd, J=6.7, 3.5 Hz, 2H), 8.74 (d, J=2.4 Hz, 1H), 8.32 (d, J=1.9 Hz, 1H), 8.09 (dd, J=7.7, 3.6 Hz, 2H), 6.97 (d, J=5.0 Hz, 1H), 6.63 (s, 1H), 2.87 (d, J=4.9 Hz, 3H), 2.54-2.42 (m, 3H). MS (ESI) m/z 473.0 [M+H]⁺

### Example 80. Synthesis of 4-(1,1-difluoro-2-methylpropan-2-yl)-N-(4-methyl-3-(7-(methylamino)-1,6-naphthyridin-3-yl)phenyl)picolinamide (Compound 84)

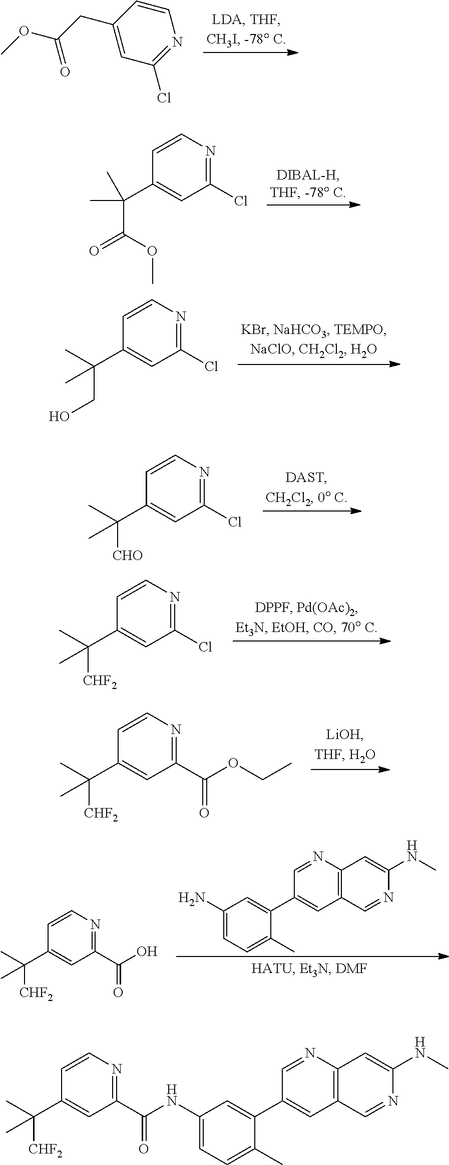

Step 1. To a solution of methyl 2-(2-chloropyridin-4-yl)acetate (1.5 g, 8.1 mmol) in tetrahydrofuran (20 mL) was added lithium diisopropylamide (1.7 g, 16.2 mmol) at −78° C. The mixture was stirred at −78° C. for 30 minutes, and then iodomethane (11.4 g, 81 mmol) was added and the mixture was stirred at −78° C. for 16 hours. The temperature of the reaction was allowed to rise to room temperature and the reaction was concentrated. The crude residue was purified by flash chromatography (silica, petroleum ether/ethyl acetate=10/1) to give methyl 2-(2-chloropyridin-4-yl)-2-methylpropanoate (1.2 g, 5.6 mmol, 70%) as a colorless oil. MS (EST) m/z 213.9 [M+H]⁺

Step 2. A solution of methyl 2-(2-chloropyridin-4-yl)-2-methylpropanoate (1.2 g, 5.6 mmol) and diisobutyl aluminium hydride (1.6 g, 11.2 mmol) in tetrahydrofuran (10 mL) was stirred at −78° C. for 16 hours. The temperature of the reaction was allowed to rise to room temperature and the reaction was concentrated. The crude residue was purified flash chromatography (silica, petroleum ether/ethyl acetate=5/1) to give 2-(2-chloropyridin-4-yl)-2-methylpropan-1-ol (600 mg, 3.24 mmol, 58%) as a colorless oil. MS (ESI) m/z 185.9 [M+H]⁺

Step 3. A solution of 2-(2-chloropyridin-4-yl)-2-methylpropan-1-ol (600 mg, 3.24 mmol), potassium bromide (77 mg, 0.65 mmol), sodium bicarbonate (55 mg, 0.65 mmol), 2,2,6,6-tetramethyl-1-piperidinyloxy (51 mg, 0.33 mmol), and sodium hypochlorite (360 mg, 0.98 mmol) in dichloromethane (10 mL) and water (2 mL) was stirred at room temperature for 2 hours. The reaction mixture was concentrated. The residue was purified by flash chromatography (silica, petroleum ether/ethyl acetate=1/1) to give 2-(2-chloropyridin-4-yl)-2-methylpropanal (249 mg, 1.36 mmol, 42%) as a colorless oil. MS (ESI) m/z 183.9 [M+H]⁺

Step 4. To a solution of 2-(2-chloropyridin-4-yl)-2-methylpropanal (249 mg, 1.36 mmol) in dichloromethane (5 mL) was added diethylaminosulphur trifluoride (1.1 g, 6.8 mmol) at 0° C. The mixture was stirred at 0° C. for 3 hours. The mixture was quenched with water (30 mL) and extracted with ethyl acetate (30 mL×3). The combined organic phases were dried over sodium sulfate, filtered, and concentrated under reduced pressure to give 2-chloro-4-(1,1-difluoro-2-methylpropan-2-yl)pyridine (210 mg, 1.02 mmol, 75%) as a colorless oil. MS (ESI) m/z 206.1 [M+H]⁺

Step 5. A solution of 2-chloro-4-(1,1-difluoro-2-methylpropan-2-yl)pyridine (210 mg, 1.02 mmol), 1,1′-bis(diphenylphosphino)ferrocene (113 mg, 0.2 mmol), palladium acetate (23 mg, 0.1 mmol) and triethylamine (0.5 mL) in ethanol (5 mL) was stirred at 70° C. for 16 hours under carbon monoxide. After cooling to room temperature, the reaction mixture was concentrated. The crude residue was purified flash chromatography (dichloromethane/methanol=1/1) to give ethyl 4-(1,1-difluoro-2-methylpropan-2-yl)picolinate (219 mg, 0.90 mmol, 88.4%) as colorless solid. MS (ESI) m/z 243.9 [M+H]⁺

Step 6. A solution of ethyl 4-(1,1-difluoro-2-methylpropan-2-yl)picolinate (219 mg, 0.90 mmol) and lithium hydroxide (65 mg, 2.7 mmol) in tetrahydrofuran (5 mL) and water (1 mL) was stirred at room temperature for 2 hours. The mixture was acidified with 1 N hydrochloric acid until pH=4 and extracted with ethyl acetate (30 mL×3). The combined organic phases were dried over sodium sulfate, filtered, and concentrated under reduced pressure to give 4-(1,1-difluoro-2-methylpropan-2-yl)picolinic acid (173 mg, 0.80 mmol, 89%) as a yellow solid. MS (ESI) m/z 216.1 [M+H]⁺

Step 7. A solution 4-(1,1-difluoro-2-methylpropan-2-yl)picolinic acid (30 mg, 0.14 mmol), 3-(5-amino-2-methylphenyl)-N-methyl-1,6-naphthyridin-7-amine (37 mg, 0.14 mmol), 1-[bis(dimethylamino)methylene]-1H-1,2,3-triazolo[4,5-b]pyridinium 3-oxid hexafluorophosphate (160 mg, 0.42 mmol), and triethylamine (0.2 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(1,1-difluoro-2-methylpropan-2-yl)-N-(4-methyl-3-(7-(methylamino)-1,6-naphthyridin-3-yl)phenyl)picolinamide (9.6 mg, 0.02 mmol, 15%) as a yellow solid. ¹H NMR (400 MHz, DMSO-d₆) δ 10.72 (s, 1H), 8.99 (s, 1H), 8.84 (d, J=2.3 Hz, 1H), 8.73 (d, J=5.2 Hz, 1H), 8.23 (dd, J=14.7, 1.6 Hz, 2H), 7.96-7.87 (m, 2H), 7.76 (dd, J=5.2, 1.8 Hz, 1H), 7.36 (d, J=8.4 Hz, 1H), 6.91 (q, J=4.8 Hz, 1H), 6.63 (s, 1H), 6.30 (t, J=56.0 Hz, 1H), 2.88 (d, J=4.9 Hz, 3H), 2.30 (s, 3H), 1.44 (s, 6H). MS (ESI)m m/z 461.7 [M+H]⁺

### Example 81. Synthesis of 4-(difluoromethyl-3-methoxy-N-(6-methyl-5-(7-(methylamino)-1,6-naphthyridin-3-yl)pyridin-3-yl)picolinamide (Compound 85)

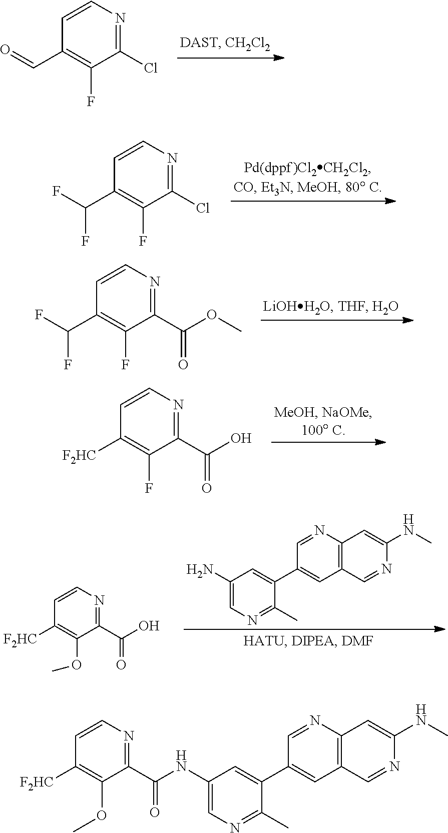

Step 1. Diethylamninosulphur trifluoride (4276 mg, 50.3 mmol) was added to a solution of 2-chloro-3-fluoroisonicotinaldehyde (2000 mg, 12.6 mmol) in dichloromethane (30 mL) at 0° C. After stirring at 25° C. for 2 hours under nitrogen, water (30 mL) was added. The reaction solution was extracted with ethyl acetate (60 mL), washed with water (20 mL) and brine (20 mL), dried over sodium sulfate, filtered, and concentrated. The residue was concentrated and purified by flash chromatography (silica, dichloromethane/methanol=20/1) to give 2-chloro-4-(difluoromethyl)-3-fluoropyridine (2000 mg, 11.1 mmol, 88%) as a white oil. MS (ESI) m/z 182 [M+H]⁺

Step 2. 1,1′-Bis(diphenylphosphino)ferrocene-palladium(II)dichloride dichloromethane complex (804.1 mg, 1.1 mmol) was added to a solution of 2-chloro-4-(difluoromethyl)-3-fluoropyridine (2000 mg, 11.1 mmol) and triethylamine (3363 mg, 33.3 mmol) in methanol (20 mL) at room temperature. The reaction mixture was stirred at 80° C. for 20 hours under carbon monoxide. After cooling to room temperature, the reaction was diluted with ethyl acetate (60 mL), washed with water (20 mL) and brine (20 mL), dried over sodium sulfate, filtered, and concentrated. The residue was purified by flash chromatography (silica, petroleum ether/ethyl acetated=2/1 then 1/1) to give methyl 4-(difluoromethyl)-3-fluoropicolinate (1200 mg, 5.9 mmol, 53%) as a brown solid. MS (ESI) m/z 206.1 [M+H]⁺

Step 3. Lithium hydroxide monohydrate (283 mg, 1.8 mmol) was added to a solution of methyl 4-(difluoromethyl)-3-fluoropicolinate (1200 mg, 5.9 mmol) in tetrahydrofuran (10 mL) and water (5 mL) at 25° C. under nitrogen. The reaction mixture was quenched with 1M hydrochloric acid until the pH was adjusted to 3-4. The mixture was extracted with ethyl acetate (80 mL). The organic layer was washed with water (20 mL) and brine (20 mL), dried over sodium sulfate, filtered, and concentrated. The residue was purified by flash chromatography (silica, petroleum ether/ethyl acetated=211 then 1/1) to give 4-(difluoromethyl)-3-fluoropicolinic acid (1100 mg, 5.8 mmol, 97.6%) as a brown solid. MS (ESI) m/z 192.1 [M+H]⁺

Step 4. 4-(Difluoromethyl)-3-fluoropicolinic acid (40 mg, 0.21 mmol) was added to a solution of sodium methoxide (33 mg, 0.63 mmol) in methanol (5 mL). The reaction mixture was stirred at 100° C. for 48 hours. The reaction mixture was quenched with 2N hydrochloric acid until the pH was adjusted to 3-4. The reaction was diluted with ethyl acetate (60 mL), washed with water (20 mL) and brine (20 mL), dried over sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give 4-(difluoromethyl)-3-methoxypicolinic acid (24 mg, 012 mmol, 57%) as a yellow solid. MS (ESI) m/z 204.1 [M+H]⁺

Step 5. 3-(5-Amino-2-methylpyridin-3-yl)-N-methyl-1,6-naphthyridin-7-amine (28 mg, 0.11 mmol) was added to a solution of 4-(difluoromethyl)-3-methoxypicolinic acid (24 mg, 0.11 mmol), N,N-diisopropylethylamine (43 mg, 0.33 mmol) and 1-[bis(dimethylamino)methylene]-1H-1,2,3-triazolo[4,5-b]pyridinium 3-oxid hexafluorophosphate (57 mg, 0.15 mmol) in N,N-dimethylformamide (2 mL) at room temperature. After stirring at room temperature for 4 hours, the reaction mixture was concentrated and the residue was purified by prep-HPLC (Column: Xbridge 21.2×250 mm C18, 10 μm, Mobile Phase A: water (10 mM ammonium bicarbonate), B: acetonitrile) to give 4-(difluoromethyl)-3-methoxy-N-(6-methyl-5-(7-(methylamino)-1,6-naphthyridin-3-yl)pyridin-3-yl)picolinamide (15 mg, 0.034 mmol, 33%) as a yellow solid. ¹H NMR (400 MHz, DMSO-d₆) δ 10.95 (s, 1H), 9.00 (s, 1H), 8.87 (dd, J=6.5, 2.4 Hz, 2H), 8.61 (d, J=4.8 Hz, 1H), 8.33 (d, J=1.9 Hz, 1H), 8.21 (d, J=2.4 Hz, 1H), 7.80 (d, J=4.8 Hz, 1H), 7.31 (s, 1H), 6.96 (q, J=4.8 Hz, 1H), 6.63 (s, 1H), 3.93 (s, 3H), 2.87 (d, J=5.0 Hz, 3H), 2.49 (s, 3H). MS (ESI) m/z 451.2 [M+H]⁺

### Example 82. Synthesis of N-(4-methyl-3-(7-(methylamino)-1,6-naphthyridin-3-yl)phenyl)-5-(2,2,2-trifluoro-1-hydroxyethyl)nicotinamide (Compound 86)

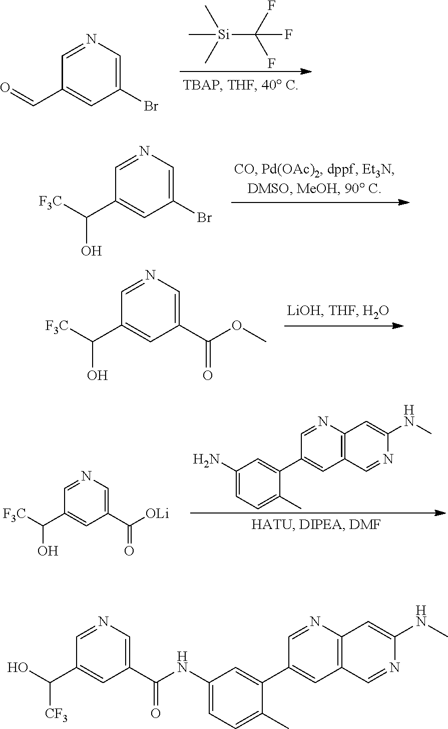

Step 1. To a solution of 5-bromonicotinaldehyde (1.0 g, 5.38 mmol) in tetrahydrofuran (20 mL) were added trimethyl(trifluoromethyl)silane (919 mg, 6.46 mmol) and tetrabutylammonium perchlorate (550 mg, 1.61 mmol) at room temperature. The mixture was stirred for 16 hours at 40° C. After cooling to room temperature, 2N hydrogen chloride (20 mL) was added to the reaction solution slowly and stirred at 25° C. for 1 hour. The resulting solution was extracted with ethyl acetate (3×50 mL). The combined organic phases were dried over sodium sulfate, filtered, and concentrated under reduced pressure. The residue was purified by flash chromatography (silica, ethyl acetate/petroleum ether=30/100) to afford 1-(5-bromopyridin-3-yl)-2,2,2-trifluoroethanol (790 mg, 3.09 mmol, 57%) as a yellow oil. MS (EST) m/z 256.0 [M+H]⁺

Step 2. A mixture of 1-(5-bromopyridin-3-yl)-2,2,2-trifluoroethanol (790 mg, 3.1 mmol), 1,1′-bis(diphenylphosphino)ferrocene (687 mg, 1.24 mmol), palladium (II) acetate (139 mg, 0.62 mmol), and triethylamine (1.25 g, 12.4 mmol) in dimethyl sulfoxide (12 mL) and methanol (8 mL) was stirred at 90° C. for 16 hours under carbon monoxide. After cooling to room temperature, the mixture was extracted with ethyl acetate (50 mL×2). The combined organic layers were washed with brine (30 mL×3), dried over anhydrous sodium sulfate, and filtered. The filtrate was concentrated under reduced pressure. The residue was purified by flash chromatography (silica, ethyl acetate in petroleum ether from 10% to 40%) to give methyl 5-(2,2,2-trifluoro-1-hydroxyethyl)nicotinate (560 mg, 2.38 mmol, 77%) as a yellow oil. MS (ESI) m/z 235.8 [M+H]⁺

Step 3. To a solution of methyl 5-(2,2,2-trifluoro-1-hydroxyethyl)nicotinate (28 mg, 0.12 mmol) in tetrahydrofuran (0.5 mL) was added lithium hydroxide monohydrate (25 mg, 7.8 mmol) in water (0.5 mL) at 25° C. After stirring at 25° C. for 2 hours, the mixture was concentrated under reduced pressure to give crude lithium 5-(2,2,2-trifluoro-1-hydroxyethyl)nicotinate (27 mg) as a white solid, which was used in the next step without further purification.

Step 4. A solution of 3-(5-amino-2-methylphenyl)-N-methyl-1,6-naphthyridin-7-amine (26 mg, 0.1 mmol), lithium 5-(2,2,2-trifluoro-1-hydroxyethyl)nicotinate (27 mg, 0.12 mmol), 1-[bis(dimethylamino)methylene]-1H-1,2,3-triazolo[4,5-b]pyridinium 3-oxid hexafluorophosphate (57 mg, 0.15 mmol), and N,N-diisopropylethylamine (52 mg, 0.4 mmol) in N,N-dimethylformamide (2 mL) was stirred at 25° C. for 3 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(4-methyl-3-(7-(methylamino)-1,6-naphthyridin-3-yl)phenyl)-5-(2,2,2-trifluoro-1-hydroxyethyl)nicotinamide (15.8 mg, 0.034 mmol, 34%) as a yellow solid. ¹H NMR (400 MHz, DMSO-d₆) δ 10.59 (s, 1H), 9.17 (d, J=2.0 Hz, 1H), 8.99 (s, 1H), 8.86 (d, J=1.5 Hz, 1H), 8.82 (d, J=2.3 Hz, 1H), 8.42 (s, 1H), 8.24 (d, J=1.9 Hz, 1H), 7.82-7.72 (m, 2H), 7.37 (d, J=9.0 Hz, 1H), 7.24 (d, J=5.8 Hz, 1H), 6.91 (q, J=4.7 Hz, 1H), 6.63 (s, 1H), 5.53-5.41 (m, 1H), 2.87 (d, J=4.9 Hz, 3H), 2.29 (s, 3H). MS (ESI) m/z 468.1 [M+H]⁺

### Example 83. Synthesis of N-(4-chloro-2-fluoro-3-(7-(methylamino)-1,6-naphthyridin-3-yl)phenyl)-4-(2-cyanopropan-2-yl)picolinamide (Compound 87)

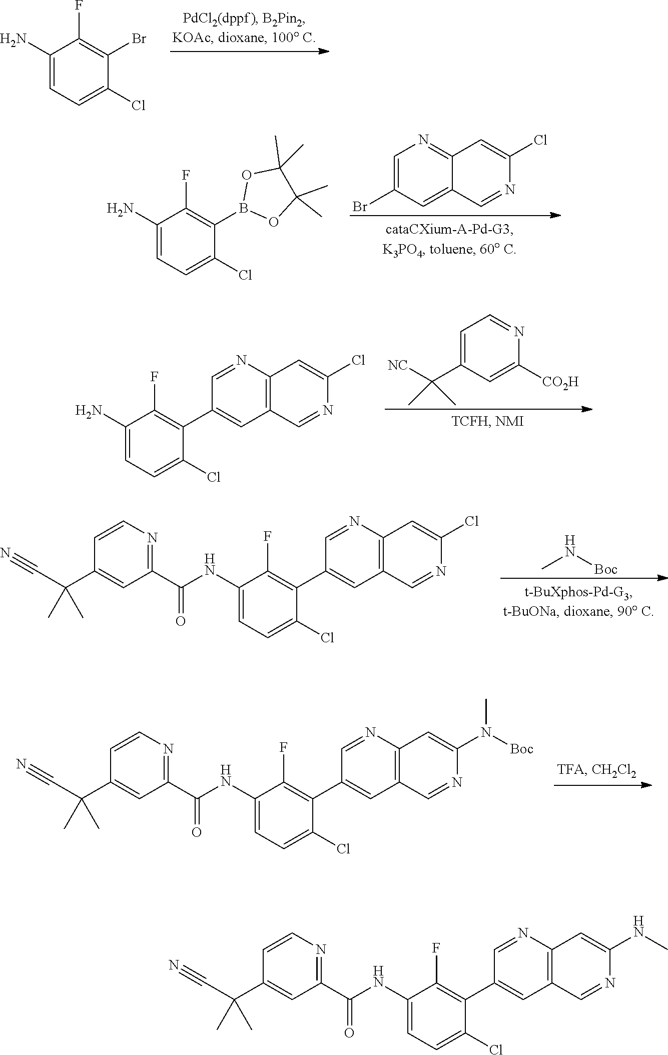

Step 1. To a solution of 3-bromo-4-chloro-2-fluoro-aniline (4 g, 17.82 mmol) and 4,4,5,5-tetramethyl-2-(4,4,5,5-tetramethyl-1,3,2-dioxaborolan-2-yl)-1,3,2-dioxaborolane (13.58 g, 53.46 mmol) in dioxane (100 mL) was added potassium acetate (7.00 g, 71.28 mmol) and 1,1′-bis(diphenylphosphino)ferrocene]dichloropalladium(II) (3.91 g, 5.35 mmol) under a nitrogen atmosphere. The mixture was stirred at 100° C. for 40 hours under a nitrogen atmosphere. On completion, the mixture was concentrated to give a residue. The residue was purified by flash chromatography (silica, petroleum ether/ethyl acetate=1/1 to 0/1) to give 4-chloro-2-fluoro-3-(4,4,5,5-tetramethyl-1,3,2-dioxaborolan-2-yl)aniline (4.8 g, 17.68 mmol, 99%) as a white solid. MS (ESI) m/z 272.1 [M+H]⁺

Step 2. To a solution of 4-chloro-2-fluoro-3-(4,4,5,5-tetramethyl-1,3,2-dioxaborolan-2-yl)aniline (2.70 g, 9.94 mmol) and 3-bromo-7-chloro-1,6-naphthyridine (1.1 g, 4.52 mmol) in toluene (45 mL) was added [(di(1-adamantyl)-butylphosphine)-2-(2′-amino-1,1′-biphenyl)]palladium(II) methanesulfonate (329 mg, 0.451 mmol) and potassium phosphate (1.5 M, 9.04 mL). The mixture solution was stirred at 60° C. for 14 hours under a nitrogen atmosphere. On completion, the solution was poured into water (50 mL) and extracted with ethyl acetate (100 mL×2). The organic layers were concentrated to give a residue. The residue was purified by flash chromatography (silica, petroleum ether/ethyl acetate=1/1 to 0/1) to give 4-chloro-3-(7-chloro-1,6-naphthyridin-3-yl)-2-fluoro-aniline (0.4 g, 1.30 mmol, 29%) as a yellow solid. MS (ESI) m/z 308.0 [M+H]⁺

Step 3. To a solution of 4-chloro-3-(7-chloro-1,6-naphthyridin-3-yl)-2-fluoro-aniline (350 mg, 1.14 mmol) and 4-(1-cyano-1-methyl-ethyl)pyridine-2-carboxylic acid (432 mg, 2.27 mmol) in acetonitrile (10 mL) was added 1-methylimidazole (NMI, 373 mg, 4.54 mmol) and chloro-N,N,N′,N′-tetramethylformamidinium hexafluorophosphate (TCFH, 637 mg, 2.27 mmol). The mixture solution was stirred at 25° C. for 2 hours. On completion, the solution was concentrated to give a residue. The residue was purified by flash chromatography (silica, petroleum ether/ethyl acetate=1/1 to 0/1) to give N-(4-chloro-3-(7-chloro-1,6-naphthyridin-3-yl)-2-fluorophenyl)-4-(2-cyanopropan-2-yl)picolinamide (0.5 g, 1.04 mmol, 92%) as a white solid. ¹H NMR (400 MHz, DMSO-d6) δ=10.63 (s, 1H), 9.43 (s, 1H), 9.23 (s, 1H), 8.84-8.80 (m, 2H), 8.25 (s, 1H), 8.23-8.20 (m, 2H), 7.89-7.88 (m, 1H), 7.67-766 (m, 1H), 1.77 (s, 6H). MS (EST) m/z 480.1 [M+H]⁺

Step 4. To a solution of N-(4-chloro-3-(7-chloro-1,6-naphthyridin-3-yl)-2-fluorophenyl)-4-(2-cyanopropan-2-yl)picolinamide (40 mg, 0.083 mmol) and tert-butyl N-methylcarbamate (16.39 mg, 0.125 mmol) in dioxane (0.4 mL) was added [(2-di-tert-butylphosphino-2′,4′,6′-triisopropyl-1,1′-biphenyl)-2-(2′-amino-1,1′-biphenyl)] palladium(II) methanesulfonate (t-BuXphos-Pd-G3, 6.62 mg, 0.008 mmol) and sodium tert-butoxide (2 M, 125 uL) under nitrogen atmosphere. The reaction mixture was stirred at 90° C. under nitrogen atmosphere for 16 hours. On completion, the solution was concentrated and the residue was purified by reversed-phase HPLC (0.1% formic acid condition) to give tert-butyl(3-(6-chloro-3-(4-(2-cyanopropan-2-yl)picolinamido)-2-fluorophenyl)-1,6-naphthyridin-7-yl)(methyl)carbamate (12 mg, 0.021 mmol, 25%) as a yellow solid. MS (ESI) m/z 575.1 [M+H]⁺

Step 5. A solution of tert-butyl(3-(6-chloro-3-(4-(2-cyanopropan-2-yl)picolinamido)-2-fluorophenyl)-1,6-naphthyridin-7-yl)(methyl)carbamate (10 mg, 0.017 mmol) in dichloromethane (0.5 mL) and trifluoroacetic acid (154 mg, 1.35 mmol, 0.1 mL) was stirred at 25° C. for 15 minutes. On completion, the solution was concentrated. The residue was purified by prep-HPLC (Column: Waters Xbridge 150×25 mm, 5 μm; Mobile Phase: A: water (ammonium bicarbonate), B: acetonitrile; B %: 48%-78%, 10 min) to give N-(4-chloro-2-fluoro-3-(7-(methylamino)-1,6-naphthyridin-3-yl)phenyl)-4-(2-cyanopropan-2-yl)picolinamide (7 mg, 13.51 μmol, 78%) as a yellow solid. 1H NMR (400 MHz, CD₃OD) δ=8.97 (s, 1H), 8.80 (s, 1H), 8.76 (d, J=5.2 Hz, 1H), 8.46-8.42 (m, 2H), 8.37 (d, J=1.2 Hz, 1H), 7.84-7.82 (m, 1H), 7.54-7.51 (m, 1H), 6.74 (s, 1H), 3.02 (s, 3H), 1.83 (s, 6H). MS (ESI) m/z 474.9 [M+H]⁺

### Example 84. Synthesis of 3-methoxy-N-(6-methyl-5-(7-(methylamino)-1,6-naphthyridin-3-yl)pyridin-3-yl)-2-(trifluoromethyl)isonicotinamide (Compound 88)

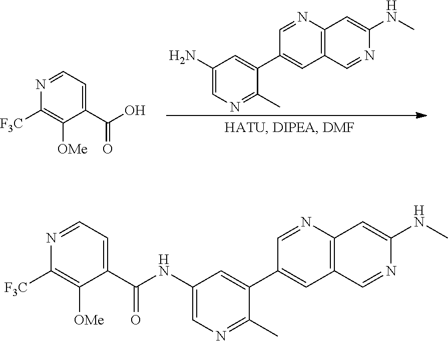 [SMILES: COc1c(C(=O)O)ccnc1C(F)(F)F ; CNc1cc2ncc(-c3cc(N)cnc3C)cc2cn1 ; CNc1cc2ncc(-c3cc(NC(=O)c4ccnc(C(F)(F)F)c4OC)cnc3C)cc2cn1]

A mixture of 3-methoxy-2-(trifluoromethyl)isonicotinic acid (96 mg, 0.43 mmol), 3-(5-amino-2-methylpyridin-3-yl)-N-methyl-1,6-naphthyridin-7-amine (79.8 mg, 0.3 mmol), 1-[bis(dimethylamino)methylene]-1H-1,2,3-triazolo[4,5-b]pyridinium 3-oxid hexafluorophosphate (245.1 mg, 0.65 mmol), and N,N-diisopropylethylamine (166.4 mg, 1.29 mmol) in N,N-dimethylformamide (3 mL) was stirred at room temperature for 2 hours. The reaction mixture was concentrated. The residue was purified by flash chromatography (silica, petroleum ether/ethyl acetate=1/1) to give 3-methoxy-N-(6-methyl-5-(7-(methylamino)-1,6-naphthyridin-3-yl)pyridin-3-yl)-2-(trifluoromethyl)isonicotinamide (14.3 mg, 0.031 mmol, 7%) as a yellow solid. ¹H NMR (500 MHz, CDCl₃) δ 9.30 (s, 1H), 8.88 (d, J=2.1 Hz, 2H), 8.67 (d, J=4.3 Hz, 2H), 8.31 (d, J=2.5 Hz, 1H), 8.13 (d, J=4.8 Hz, 1H), 8.05 (d, J=2.1 Hz, 1H), 6.80 (s, 1H), 4.05 (s, 3H), 3.05 (d, J=2.8 Hz, 3H), 2.58 (s, 3H). MS (ESI) m/z 469.0 [M+H]⁺

### Example 85. Synthesis of (S)-4-(1,1-difluoropropan-2-yl)-N-(4-methyl-3-(7-(methylamino)-1,6-naphthyridin-3-yl)phenyl)picolinamide and (R)-4-(1,1-difluoropropan-2-yl)-N-(4-methyl-3-(7-(methylamino)-1,6-naphthyridin-3-yl)phenyl)picolinamide (Compounds 89 and 90)

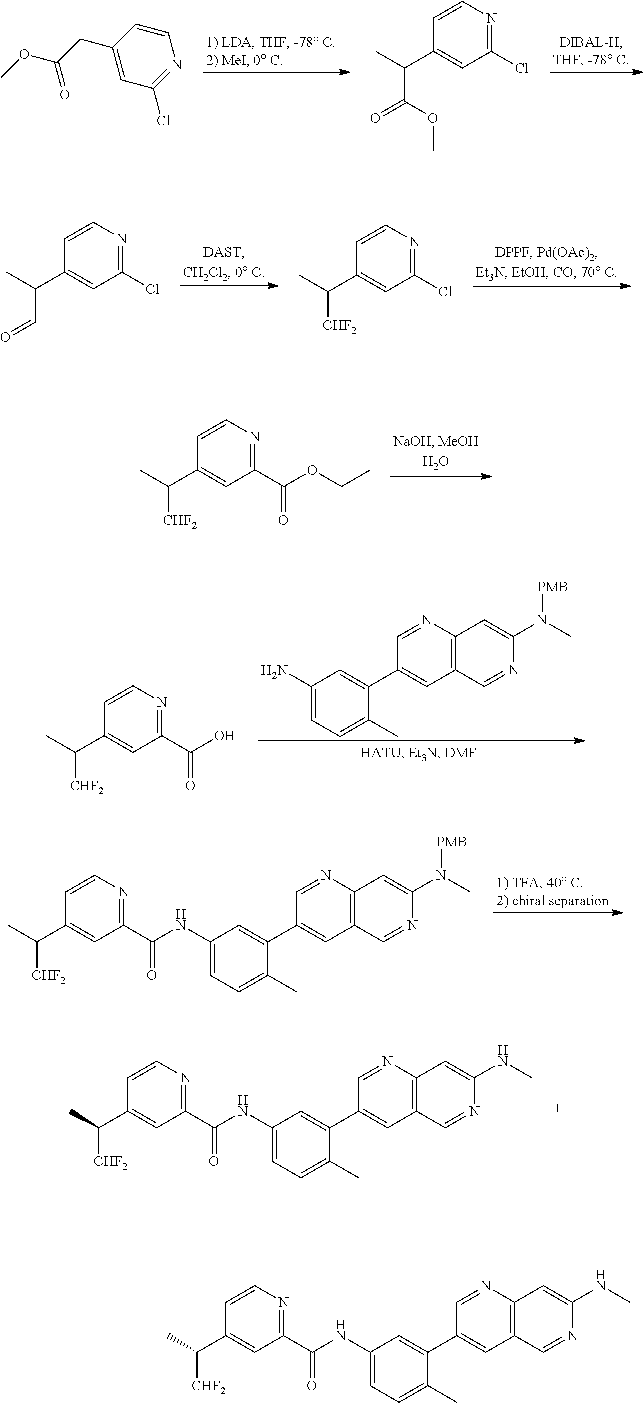

Step 1. To a solution of methyl 2-(2-chloropyridin-4-yl)acetate (3.8 g, 20.5 mmol) in tetrahydrofuran (40 mL) was added lithium diisopropylamide (11.3 mL, 22.6 mmol, 2 M in tetrahydrofuran) at −78° C. The mixture was stirred at −78° C. for 0.5 hour. Iodomethane (3.2 g, 22.6 mmol) was added at −78° C. and the reaction mixture was stirred at 0° C. for 0.5 hour. The reaction mixture was quenched by water (150 mL) and extracted with ethyl acetate (100 mL×2). The combined organic phases were concentrated and purified by flash chromatography (silica, petroleum ether/ethyl acetate=5/1) to give methyl 2-(2-chloropyridin-4-yl)propanoate (3.8 g, 19.1 mmol, 932) as a yellow oil. MS (ESI) m/z 200.1 [M+H]⁺

Step 2. To a solution of methyl 2-(2-chloropyridin-4-yl)propanoate (2.8 g, 14.1 mmol) in dichloromethane (10 mL) was added diisobutylaluminium hydride (15.5 mL, 15.5 mmol, 1 M in cyclohexane) at −78° C. The mixture was stirred at −78° C. for 5 hours. The reaction mixture was quenched by sodium sulfate decahydrate (5 g) and stirred at room temperature for 1 hour. The mixture was filtered, washed with ethyl acetate (30 mL). The filtrate was concentrated and purified by flash chromatography (silica, petroleum ether/ethyl acetate=4/3) to give 2-(2-chloropyridin-4-yl)propanal (500 mg, 2.96 mmol, 21%) as a yellow oil. MS (ESI) m/z 169.8 [M+H]⁺

Step 3. To a solution of 2-(2-chloropyridin-4-yl)propanal (500 mg, 2.96 mmol) in dichloromethane (10 mL) was added diethylaminosulfur trifluoride (DAST, 1.19 g, 7.4 mmol) at 0° C. The mixture was stirred at 0° C. for 2 hours. The reaction mixture was quenched by water (20 mL) and extracted with dichloromethane (20 mL×2). The combined organic phases were concentrated and purified by flash chromatography (silica, petroleum ether/ethyl acetate=3/1) to give 2-chloro-4-(1,1-difluoropropan-2-yl)pyridine (230 mg, 1.2 mmol, 41%) as a yellow oil. MS (ESI) m/z 192.1 [M+H]⁺

Step 4. A solution of 2-chloro-4-(1,1-difluoropropan-2-yl)pyridine (230 mg, 1.2 mmol), 1,1′-bis(diphenylphosphino)ferrocene (133 mg, 0.24 mmol), palladium (II) acetate (27 mg, 0.12 mmol) and triethylamine (365 mg, 3.61 mmol) in ethanol (10 mL) was stirred at 70° C. for 18 hours under carbon monoxide atmosphere. The reaction was cooled to room temperature and concentrated. The residue was purified by flash chromatography (silica, petroleum ether/ethyl acetate=4/1) to give ethyl 4-(1,1-difluoropropan-2-yl)picolinate (170 mg, 0.74 mmol, 62%) as a yellow oil. MS (ESI) m/z 230.1 [M+H]⁺

Step 5. A mixture of ethyl 4-(1,1-difluoropropan-2-yl)picolinate (170 mg, 0.74 mmol) and sodium hydroxide (89 mg, 2.23 mmol) in methanol (5 mL) and water (2 mL) was stirred at room temperature for 2 hours. The reaction mixture was adjusted pH=4 with hydrochloric acid (1 N) and concentrated. The residue was purified by flash chromatography (silica, dichloromethane/methanol=10/1) to give 4-(1,1-difluoropropan-2-yl)picolinic acid (110 mg, 0.55 mmol, 74%) as a yellow solid. MS (ESI) m/z 202.1 [M+H]⁺

Step 6. A mixture of 4-(1,1-difluoropropan-2-yl)picolinic acid (75 mg, 0.37 mmol), 3-(5-amino-2-methylphenyl)-N-(4-methoxybenzyl)-N-methyl-1,6-naphthyridin-7-amine (143 mg, 0.37 mmol), 1-[bis(dimethylamino)methylene]-1H-1,2,3-triazolo[4,5-b]pyridinium 3-oxid hexafluorophosphate (170 mg, 0.45 mmol), and triethylamine (113 mg, 1.12 mmol) in N,N-dimethylformamide (3 mL) was stirred at room temperature for 2 hours. The reaction mixture was treated with water (50 mL), and the precipitate was filtered and washed with water (5 mL). The solid was purified by flash chromatography (silica, petroleum ether/ethyl acetate=1/1) to give 4-(1,1-difluoropropan-2-yl)-N-(3-(7-((4-methoxybenzyl)(methyl)amino)-1,6-naphthyridin-3-yl)-4-methylphenyl)picolinamide (110 mg, 0.19 mmol, 52%) as a yellow solid. MS (ESI) m/z 568.0 [M+H]⁺

Step 7. A solution of 4-(1,1-difluoropropan-2-yl)-N-(3-(7-((4-methoxybenzyl)(methyl)amino)-1,6-naphthyridin-3-yl)-4-methylphenyl)picolinamide (110 mg, 0.19 mmol) in trifluoroacetic acid (2 mL) was stirred at 40° C. for 2 hours. The reaction mixture was concentrated. The residue was purified by flash chromatography (silica, dichloromethane/methanol=10/1) to give 4-(1,1-difluoropropan-2-yl)-N-(4-methyl-3-(7-(methylamino)-1,6-naphthyridin-3-yl)phenyl)picolinamide (80 mg, 0.18 mmol, 92%) as a yellow solid. The enantiomers were separated by chiral SFC (Instrument: SFC-150 (Thar, Waters); Column: OJ 20×250 mm, 10 μm (Daicel); Mobile phase: CO₂/MeOH (0.2% methanol ammonia)=50/50; Detection wavelength: 214 nm) to afford the first eluting enantiomer (retention time 9.8 min) which was arbitrarily assigned as (S)-4-(1,1-difluoropropan-2-yl)-N-(4-methyl-3-(7-(methylamino)-1,6-naphthyridin-3-yl)phenyl)picolinamide (23 mg, 0.05 mmol, 27%) as a yellow solid. ¹H NMR (500 MHz, DMSO-d₆) δ 10.69 (s, 1H), 8.98 (s, 1H), 8.83 (d, J=2.3 Hz, 1H), 8.71 (d, J=5.1 Hz, 1H), 8.25 (d, J=2.1 Hz, 1H), 8.14 (s, 1H), 7.96-7.85 (m, 2H), 7.66 (dd, J=5.0, 1.6 Hz, 1H), 7.35 (d, J=8.4 Hz, 1H), 6.94-6.84 (m, 1H), 6.63 (s, 1H), 6.32 (td, J=56.0, 4.2 Hz, 1H), 3.55 (dd, J=16.7, 12.6 Hz, 1H), 2.87 (d, J=4.9 Hz, 3H), 2.29 (s, 3H), 1.36 (dd, J=10.6, 7.9 Hz, 3H). MS (ESI) m/z 448.1 [M+H]⁺;
and the second eluting enantiomer (retention time 11.6 min) which was arbitrarily assigned as (R)-4-(1,1-difluoropropan-2-yl)-N-(4-methyl-3-(7-(methylamino)-1,6-naphthyridin-3-yl)phenyl)picolinamide (14.3 mg, 0.032 mmol, 16%) as a yellow solid. ¹H NMR (500 MHz, DMSO-d₆) δ 10.70 (s, 1H), 9.01 (s, 1H), 8.85 (s, 1H), 8.71 (d, J=5.0 Hz, 1H), 8.30 (s, 1H), 8.14 (s, 1H), 7.97-7.83 (m, 2H), 7.72-7.61 (m, 1H), 7.36 (d, J=8.4 Hz, 1H), 7.00 (s, 1H), 6.63 (s, 1H), 6.32 (td, J=56.0, 4.2 Hz, 1H), 3.61-3.47 (m, 1H), 2.88 (s, 3H), 2.30 (s, 3H), 1.37 (d, J=7.1 Hz, 3H). MS (EST) m/z 448.1 [M+H]⁺

### Example 86. Synthesis of (S)-4-(1,1-difluoropropan-2-yl)-N-(6-methyl-5-(7-(methylamino)-1,6-naphthyridin-3-yl)pyridin-3-yl)picolinamide and (R)-4-(1,1-difluoropropan-2-yl)-N-(6-methyl-5-(7-(methylamino)-1,6-naphthyridin-3-yl)pyridin-3-yl)picolinamide (Compounds 91 and 92)

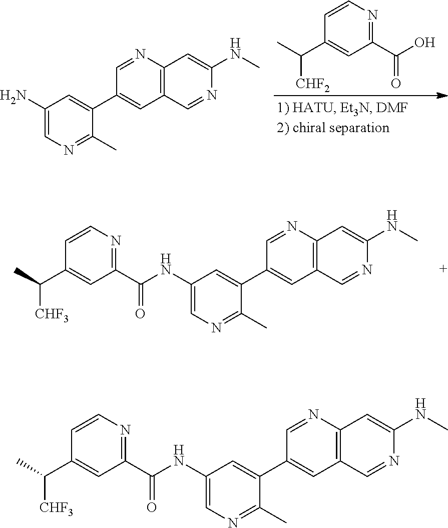

A mixture of 4-(1,1-difluoropropan-2-yl)picolinic acid (40 mg, 0.2 mmol), 3-(5-amino-2-methylpyridin-3-yl)-N-methyl-1,6-naphthyridin-7-amine (53 mg, 0.2 mmol), 1-[bis(dimethylamino)methylene]-1H-1,2,3-triazolo[4,5-b]pyridinium 3-oxid hexafluorophosphate (91 mg, 0.24 mmol), and triethylamine (40 mg, 0.4 mmol) in N,N-dimethylformamide (2 mL) was stirred at room temperature for 2 hours. The reaction mixture was treated with water (50 mL), and extracted with ethyl acetate (30 mL×2). The combined organic phases were concentrated and purified by flash chromatography (silica, dichloromethane/methanol=10/1) to give 4-(1,1-difluoropropan-2-yl)-N-(6-methyl-5-(7-(methylamino)-1,6-naphthyridin-3-yl)pyridin-3-yl)picolinamide (45 mg, 0.1 mmol, 50%) as a yellow solid. The enantiomers were separated by chiral SFC (Instrument: SFC-150 (Waters); Column: OJ 20×250 mm, 10 μm (Daicel); Mobile phase: CO₂/MeOH (0.2% methanol ammonia)=50/50; Detection wavelength: 214 nm) to afford the first eluting enantiomer (retention time 5.1 min) which was arbitrarily assigned as (S)-4-(1,1-difluoropropan-2-yl)-N-(6-methyl-5-(7-(methylamino)-1,6-naphthyridin-3-yl)pyridin-3-yl)picolinamide (19 mg, 0.042 mmol, 21%) as a yellow solid. ¹H NMR (500 MHz, DMSO-d₆) δ 10.97 (s, 1H), 9.06 (d, J=2.4 Hz, 1H), 9.00 (s, 1H), 8.88 (d, J=2.3 Hz, 1H), 8.74 (d, J=5.0 Hz, 1H), 8.39-8.28 (m, 2H), 8.16 (s, 1H), 7.68 (dd, J=4.9, 1.7 Hz, 1H), 6.99 (s, 1H), 6.64 (s, 1H), 6.46-6.18 (m, 1H), 3.55 (s, 1H), 2.88 (d, J=4.3 Hz, 3H), 2.49 (s, 3H), 1.37 (d, J=7.2 Hz, 3H). MS (ESI) m/z 449.1 [M+H]⁺;
and the second eluting enantiomer (retention time 6.7 min) which was arbitrarily assigned as (R)-4-(1,1-difluoropropan-2-yl)-N-(6-methyl-5-(7-(methylamino)-1,6-naphthyridin-3-yl)pyridin-3-yl)picolinamide (22.5 mg, 0.05 mmol, 25%) as a yellow solid. ¹H NMR (500 MHz, DMSO-d₆) δ 10.98 (s, 1H), 9.06 (d, J=2.4 Hz, 1H), 9.00 (s, 1H), 8.88 (d, J=2.3 Hz, 1H), 8.74 (d, J=5.0 Hz, 1H), 8.39-8.27 (m, 2H), 8.16 (s, 1H), 7.68 (dd, J=5.0, 1.6 Hz, 1H), 6.99 (s, 1H), 6.64 (s, 1H), 6.49-6.14 (m, 1H), 3.55 (s, 1H), 2.88 (d, J=4.3 Hz, 3H), 2.49 (s, 3H), 1.37 (d, J=7.2 Hz, 3H). MS (ESI) m/z 449.1 [M+H]⁺

### Example 87. Synthesis of (R)-4-(1-(difluoromethoxy)ethyl)-N-(4-methyl-3-(7-(methylamino)-1,6-naphthyridin-3-yl)phenyl)picolinamide and (S)-4-(1-(difluoromethoxy)ethyl)-N-(4-methyl-3-(7-(methylamino)-1,6-naphthyridin-3-yl)phenyl)picolinamide (Compounds 93 and 94)

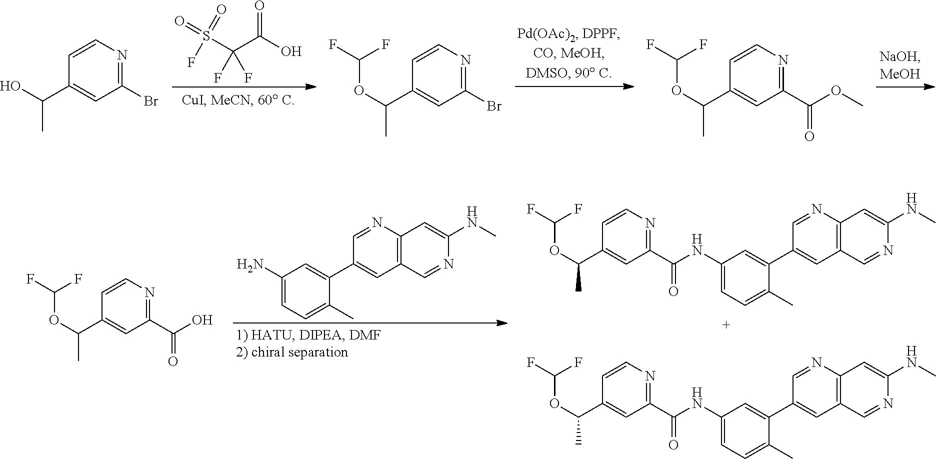

Step 1. A solution of 1-(2-bromopyridin-4-yl)ethan-1-ol (1.6 g, 7.96 mmol) and copper iodide (151 mg, 079 mmol) in acetonitrile (10 mL) was stirred at 60° C. for 10 minutes. Then 2,2-difluoro-2-(fluorosulfonyl)acetic acid (2.88 g, 1.92 mmol) was added. After stirring at 60° C. for 16 hours under nitrogen, the reaction was quenched with ice water (20 mL) and extracted with ethyl acetate (30 mL×2). The combined organic phases were concentrated and purified by flash chromatography (silica, petroleum ether/ethyl acetate=1/1) to give 2-bromo-4-(1-(difluoromethoxy)ethyl)pyridine (0.7 g, 2.79 mmol, 35%) as a yellow oil. MS (ESI) m/z 251.9 [M+H]⁺

Step 2. A solution of 2-bromo-4-(1-(difluoromethoxy)ethyl)pyridine (700 mg, 2.78 mmol), 1,1′-bis(diphenylphosphino)ferrocene (615 mg, 1.11 mmol), palladium (II) acetate (124 mg, 0.56 mmol), and triethylamine (280 mg, 8.34 mmol) in dimethyl sulfoxide (6 mL) and methanol (4 mL) was stirred at 90° C. for 18 hours under a carbon monoxide atmosphere. The reaction was cooled to room temperature and concentrated. The residue was quenched with water (15 mL) and extracted with ethyl acetate (30 mL×2). The combined organic phases were concentrated and purified by flash chromatography (silica, petroleum ether/ethyl acetate=1/1) to give methyl 4-(1-(difluoromethoxy)ethyl)picolinate (0.2 g, 0.87 mmol, 31%) as a brown oil. MS (ESI) m/z 223.1 [M+H]⁺

Step 3. A mixture of methyl 4-(1-(difluoromethoxy)ethyl)picolinate (200 mg, 0.87 mmol) and sodium hydroxide (2N in water, 0.87 mL, 1.74 mmol,) in methanol (5 mL) was stirred at room temperature for 1 hour. The reaction mixture was extracted with ethyl acetate (30 mL×2). The combined aqueous layers were adjusted to pH=4 with hydrochloric acid (6 N) and extracted with ethyl acetate (30 mL×2). The combined organic phases were concentrated to give 4-(1-(difluoromethoxy)ethyl)picolinic acid (120 mg. 0.55 mmol, 64%) as a brown oil. MS (ESI) m/z 218.1 [M+H]⁺

Step 4. A mixture of 4-(1-(difluoromethoxy)ethyl)picolinic acid (55 mg, 0.25 mmol), 3-(5-amino-2-methylphenyl)-N-methyl-1,6-naphthyridin-7-amine (66 mg, 0.25 mmol), 1-[bis(dimethylamino)methylene]-1H-1,2,3-triazolo[4,5-b]pyridinium 3-oxid hexafluorophosphate (104 mg, 0.27 mmol), and N,N-diisopropylethylamine (64 mg, 0.5 mmol) in N,N-dimethylformamide (3 mL) was stirred at room temperature for 1 hour. The reaction mixture was treated with water (50 mL). The precipitate was collected by filtration, washed with water (5 mL) and dried to give 4-(1-(difluoromethoxy)ethyl)-N-(4-methyl-3-(7-(methylamino)-1,6-naphthyridin-3-yl)phenyl)picolinamide (100 mg, 0.22 mmol, 100%) as a yellow solid. MS (ESI) m/z 464.0 [M+H]. The enantiomers were separated by chiral SFC (Instrument: SFC-150 (Waters); Column: OJ 20×250 mm, 10 μm (Daicel); Mobile phase: CO₂/MeOH (0.2% methanol ammonia)=40/60; Detection wavelength: 214 nm) to afford the first eluting enantiomer (retention time 4.3 min) which was arbitrarily assigned as (R)-4-(1-(difluoromethoxy)ethyl)-N-(4-methyl-3-(7-(methylamino)-1,6-naphthyridin-3-yl)phenyl)picolinamide (22.4 mg, 0.048 mmol, 22.4%) as a yellow solid. ¹H NMR (500 MHz, DMSO-d₆) δ 10.70 (s, 1H), 8.98 (s, 1H), 8.83 (d, J=2.5 Hz, 1H), 8.74 (d, J=5.0 Hz, 1H), 8.24 (d, J=2.0 Hz, 1H), 8.15 (d, J=1.5 Hz, 1H), 7.92 (d, J=2.5 Hz, 1H), 7.88 (dd, J=8.0, 2.0 Hz, 1H), 7.67 (dd, J=5.0, 1.5 Hz, 1H), 7.35 (d, J=8.0 Hz, 1H), 6.98-6.68 (m, 2H), 6.63 (s, 1H), 5.50 (q, J=6.5 Hz, 1H), 2.87 (d, J=4.5 Hz, 3H), 2.29 (s, 3H), 1.53 (d, J=7.0 Hz, 3H). MS (ESI) m/z 464.2[M+H]⁺;
and the second eluting enantiomer (retention time 8.8 min) which was arbitrarily assigned as (S)-4-(1-(difluoromethoxy)ethyl)-N-(4-methyl-3-(7-(methylamino)-1,6-naphthyridin-3-yl)phenyl)picolinamide (39.2 mg, 0.085 mmol, 39.2%) as a yellow solid. ¹H NMR (500 MHz, DMSO-d₆) δ 10.70 (s, 1H), 8.99 (s, 1H), 8.84 (d, J=2.0 Hz, 1H), 8.74 (d, J=5.0 Hz, 1H), 8.27 (s, 1H), 8.14 (s, 1H), 7.93 (d, J=1.5 Hz, 1H), 7.89 (dd, J=8.0, 2.0 Hz, 1H), 7.67 (dd, J=5.5, 2.0 Hz, 1H), 7.35 (d, J=8.0 Hz, 1H), 6.98-6.68 (m, 2H), 6.63 (s, 1H), 5.50 (q, J=5.0 Hz, 1H), 2.87 (d, J=4.0 Hz, 3H), 2.29 (s, 3H), 1.53 (d, J=6.0 Hz, 3H). MS (ESI) m/z 464.1 [M+H]⁺

### Example 88. Synthesis of 4-(difluoromethoxy)-N-(6-methyl-5-(7-(methylamino)-1,6-naphthyridin-3-yl)pyridin-3-yl)picolinamide (Compound 95)

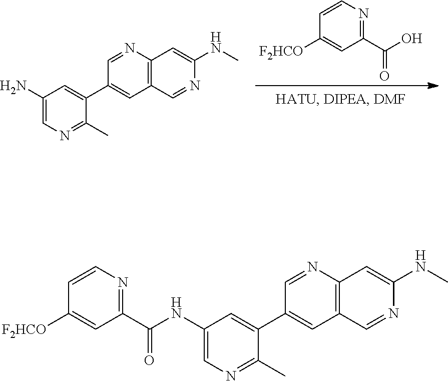 [SMILES: CNc1cc2ncc(-c3cc(N)cnc3C)cc2cn1 ; O=C(O)c1cc(OC(F)F)ccn1 ; CNc1cc2ncc(-c3cc(NC(=O)c4cc(OC(F)F)ccn4)cnc3C)cc2cn1]

A mixture of 4-(difluoromethoxy)picolinic acid (74 mg, 0.39 mmol), 3-(5-amino-2-methylpyridin-3-yl)-N-methyl-1,6-naphthyridin-7-amine (72.6 mg, 0.27 mmol), 1-[bis(dimethylamino)methylene]-1H-1,2,3-triazolo[4,5-b]pyridinium 3-oxid hexafluorophosphate (222.3 mg, 0.59 mmol), and N,N-diisopropylethylamine (151 mg, 1.17 mmol) in N,N-dimethylformamide (3 mL) was stirred at room temperature for 2 hours. The reaction mixture was concentrated. The residue was purified by prep-HPLC (SunFire C18, 4.6×50 mm, 3.5 μm, Mobile Phase: A: water (0.01% ammonium bicarbonate), B: acetonitrile; B %: 5%-95% in 1.5 min) to give 4-(difluoromethoxy)-N-(6-methyl-5-(7-(methylamino)-1,6-naphthyridin-3-yl)pyridin-3-yl)picolinamide (17.2 mg, 0.039 mmol, 10%) as a yellow solid. ¹H NMR (500 MHz, CDCl₃) δ 10.07 (s, 1H), 8.92-8.85 (m, 2H), 8.77 (d, J=2.5 Hz, 1H), 8.61 (d, J=5.5 Hz, 1H), 8.38 (d, J=2.5 Hz, 1H), 8.06 (d, J=1.7 Hz, 1H), 8.00 (d, J=2.2 Hz, 1H), 7.26-7.21 (m, 2H), 6.91-6.53 (m, 2H), 3.05 (d, J=5.3 Hz, 3H), 2.58 (s, 3H). MS (EST) m/z 437.1[M+H]⁺

### Example 89. Synthesis of 2-isopropyl-3-methoxy-N-(6-methyl-5-(7-(methylamino)-1,6-naphthyridin-3-yl)pyridin-3-yl)isonicotinamide (Compound 96)

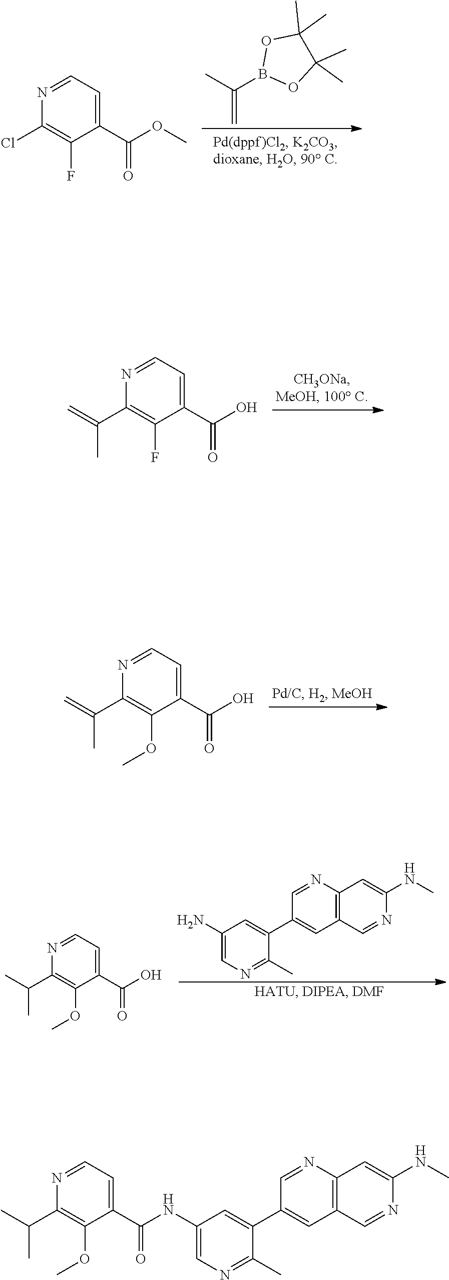

Step 1. A mixture of methyl 2-chloro-3-fluoroisonicotinate (189 mg, 1.0 mmol), 4,4,5,5-tetramethyl-2-(prop-1-en-2-yl)-1,3,2-dioxaborolane (252 mg, 1.5 mmol), [1,1′-bis(diphenylphosphino)ferrocene]dichloropalladium(II) (82 mg, 0.1 mmol) and potassium carbonate (276 mg, 2.0 mmol) in water (1.5 mL) and 1,4-dioxane (6 mL) was stirred at 90° C. for 16 hours under nitrogen. After cooling to room temperature, the organic solvent was removed. The residue was washed with ethyl acetate (20 mL). The aqueous phase was acidified with 1 N hydrogen chloride till no more precipitate formed. The mixture was filtered. The solid was washed with water (20 mL) and dried to give 3-fluoro-2-(prop-1-en-2-yl)isonicotinic acid (100 mg. 0.55 mmol, 55%) as a white solid. MS (ESI) m/z 182.1 [M+H]⁺

Step 2. A solution of 3-fluoro-2-(prop-1-en-2-yl)isonicotinic acid (100 mg, 0.55 mmol) and sodium methanolate (149 mg, 2.76 mmol) in methanol (10 mL) was stirred at 100° C. for 72 hours. The mixture was filtered. The filtrate was concentrated in vacuo to give 3-methoxy-2-(prop-1-en-2-yl)isonicotinic acid (85 mg, 0.44 mmol, 48%) as a white solid. MS (ESI) m/z 194.1 [M+H]⁺

Step 3. A mixture of 3-methoxy-2-(prop-1-en-2-yl)isonicotinic acid (85 mg, 0.44 mmol) and palladium (10% on activated carbon, 8 mg) in methanol (5 mL) was stirred at room temperature for 2 hours under hydrogen. The mixture was filtered and the filtrate was concentrated. The mixture was purified by prep-HPLC (Column: Xbridge 21.2×250 mm C18, 10 μm, Mobile Phase A: water (10 mM ammonium bicarbonate), B: acetonitrile) to give 2-isopropyl-3-methoxyisonicotinic acid (20 mg, 0.1 mmol, 23%) as a white solid. MS (ESI) m/z 196.1 [M+H]⁺

Step 4. A mixture of 2-isopropyl-3-methoxyisonicotinic acid (20 mg, 0.1 mmol), 3-(5-amino-2-methylpyridin-3-yl)-N-methyl-1,6-naphthyridin-7-amine (27 mg, 0.1 mmol), 1-[bis(dimethylamino)methylene]-1H-1,2,3-triazolo[4,5-b]pyridinium 3-oxid hexafluorophosphate (46 mg, 0.12 mmol), and N,N-diisopropylethylamine (26 mg, 0.2 mmol) in N,N-dimethylformamide (3 mL) was stirred at room temperature for 4 hours. The mixture was purified by prep-HPLC (Column: Xbridge 21.2×250 mm C18, 10 μm, Mobile Phase A: water (10 mM ammonium bicarbonate), B: acetonitrile) to give 2-isopropyl-3-methoxy-N-(6-methyl-5-(7-(methylamino)-1,6-naphthyridin-3-yl)pyridin-3-yl)isonicotinamide (34.5 mg, 0.08 mmol, 78%) as a yellow solid. ¹H NMR (500 MHz, DMSO-d₆) δ 10.78 (s, 1H), 9.00 (s, 1H), 8.86 (d, J=2.0 Hz, 1H), 8.80 (d, J=2.5 Hz, 1H), 8.43 (d, J=4.5 Hz, 1H), 8.32 (d, J=2.5 Hz, 1H), 8.14 (d, J=2.0 Hz, 1H), 8.38 (d, J=4.5 Hz, 1H), 6.98-6.95 (m, 1H), 6.64 (s, 1H), 3.82 (s, 3H), 3.49-3.45 (m, 1H), 2.88 (d, J=4.5 Hz, 3H), 2.48 (s, 3H), 1.24 (d, J=7.0 Hz, 6H). MS (ESI) m/z 443.2 [M+H]⁺

### Example 90. Synthesis of 3-fluoro-4-isopropyl-N-(6-methyl-5-(7-(methylamino)-1,6-naphthyridin-3-yl)pyridin-3-yl)picolinamide (Compound 97)

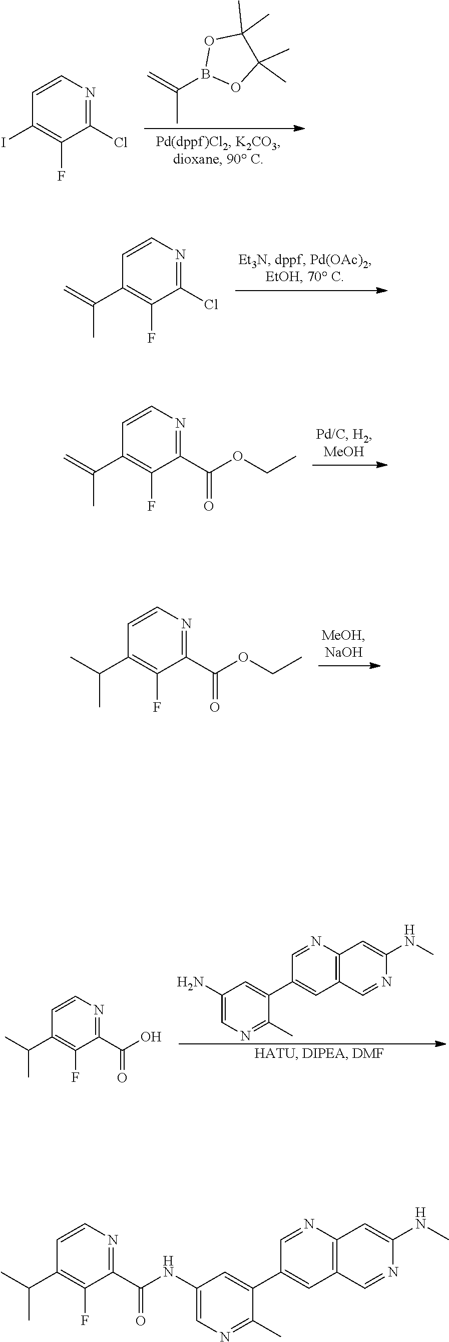

Step 1. A solution of 2-chloro-3-fluoro-4-iodopyridine (500 mg, 1.95 mmol), 4,4,5-trimethyl-2-(prop-1-en-2-yl)-1,3,2-dioxaborolane (300 mg, 1.95 mmol), [1,1′-bis(diphenylphosphino)ferrocene]dichloropalladium(II) (139 mg, 0.19 mmol) and potassium carbonate (538 mg, 3.90 mmol) in 1,4-dioxane (8 mL) was stirred at 90° C. for 2 hours under nitrogen. The mixture was diluted with water (100 mL) and extracted with ethyl acetate (100 mL×3). The combined organic layers were washed with brine (10 mL), dried over anhydrous sodium sulfate, filtered, and concentrated. The residue was purified by flash chromatography (silica, petroleum ether/ethyl acetate=1/1) to give 2-chloro-3-fluoro-4-(prop-1-en-2-yl)pyridine (300 mg, 1.75 mmol, 91) as a yellow solid. MS (ESI) m/z 172.7 [M+H]⁺

Step 2. A mixture of 2-chloro-3-fluoro-4-(prop-1-en-2-yl)pyridine (300 mg, 1.75 mmol), palladium(II)acetate (84 mg, 0.17 mmol), 1,1′-bis(diphenylphosphino)ferrocene (183 mg, 0.34 mmol) and triethylamine (353 mg, 3.5 mmol) in ethanol (6 mL) was stirred at 70° C. for 16 hours under the protection of carbon monoxide. After cooling to room temperature, the reaction mixture was diluted with water (100 mL) and extracted with ethyl acetate (100 mL×3). The combined organic phases were washed brine (100 mL×2), dried over sodium sulfate, filtered, and concentrated. The residue was purified by flash chromatography (silica, petroleum ether/ethyl acetate=1/2) to give ethyl 3-fluoro-4-(prop-1-en-2-yl)picolinate (250 mg, 1.19 mmol, 68) as a yellow solid. MS (ESI) m/z 210.1[M+H]⁺

Step 3. A mixture of ethyl 3-fluoro-4-(prop-1-en-2-yl)picolinate (250 mg, 1.19 mmol) and palladium (10% on carbon, 631 mg, 5.95 mmol) in methanol (5 mL) was stirred at room temperature for 2 hours under hydrogen. The reaction mixture was diluted with water (50 mL) and extracted with ethyl acetate (50 mL×3). The combined organic phases were washed with brine (50 mL×2), dried over sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give ethyl 3-fluoro-4-isopropylpicolinate (200 mg, 0.95 mmol, 80%) as a yellow solid. MS (ESI) m/z 211.9 [M+H]⁺

Step 4. A solution of ethyl 3-fluoro-4-isopropylpicolinate (100 mg, 0.47 mmol) and sodium hydroxide (38 mg, 0.95 mmol) in methanol (3 mL) was stirred at room temperature for 2 hours. The solvent was evaporated under reduced pressure, and the residue was diluted with water (5 mL). The mixture was adjusted to pH=3 by adding 1 N hydrochloric acid slowly and then was extracted with ethyl acetate (10 mL×3). The combined organic phases were dried over sodium sulfate, filtered, and concentrated under reduced pressure to give 3-fluoro-4-isopropylpicolinic acid (80 mg, 0.43 mmol, 96%) as a yellow solid. MS (ESI) m/z 184.1 [M+H]⁺

Step 5. A solution of 3-fluoro-4-isopropylpicolinic acid (30 mg, 0.16 mmol), 3-(5-amino-2-methylpyridin-3-yl)-N-methyl-1,6-naphthyridin-7-amine (43 mg, 0.16 mmol), 1-[bis(dimethylamino)methylene]-1H-1,2,3-triazolo[4,5-b]pyridinium 3-oxid hexafluorophosphate (91 mg, 0.24 mmol), and N,N-diisopropylethylamine (62 mg, 0.48 mmol) in N,N-dimethylformamide (3 mL) was stirred at room temperature for 2 hours. The mixture was washed with brine and extracted with ethyl acetate (10 mL×3). The combined organic phases were dried over sodium sulfate, filtered, and concentrated under reduced pressure to give 3-fluoro-4-isopropyl-N-(6-methyl-5-(7-(methylamino)-1,6-naphthyridin-3-yl)pyridin-3-yl)picolinamide (16.8 mg, 0.039 mmol, 24%) as a yellow solid. ¹H NMR (500 MHz, DMSO-d₆) δ 10.89 (s, 1H), 9.00 (s, 1H), 8.92 (s, 1H), 8.88 (s, 1H), 8.49 (d, J=4.8 Hz, 1H), 8.35 (s, 1H), 8.24 (s, 1H), 7.69 (t, J=5.0 Hz, 1H), 7.00 (s, 1H), 6.63 (s, 1H), 3.28-3.24 (m, 1H), 2.88 (d, J=3.5 Hz, 3H), 2.49-2.48 (m, 3H), 1.27 (d, J=6.9 Hz, 6H). MS (EST) m/z 431.1 [M+H]⁺

### Example 91. Synthesis of -fluoro-4-isopropyl-N-(6-methyl-5-(7-(methylamino)-1,6-naphthyridin-3-yl)pyridin-3-yl)picolinamide (Compound 98)

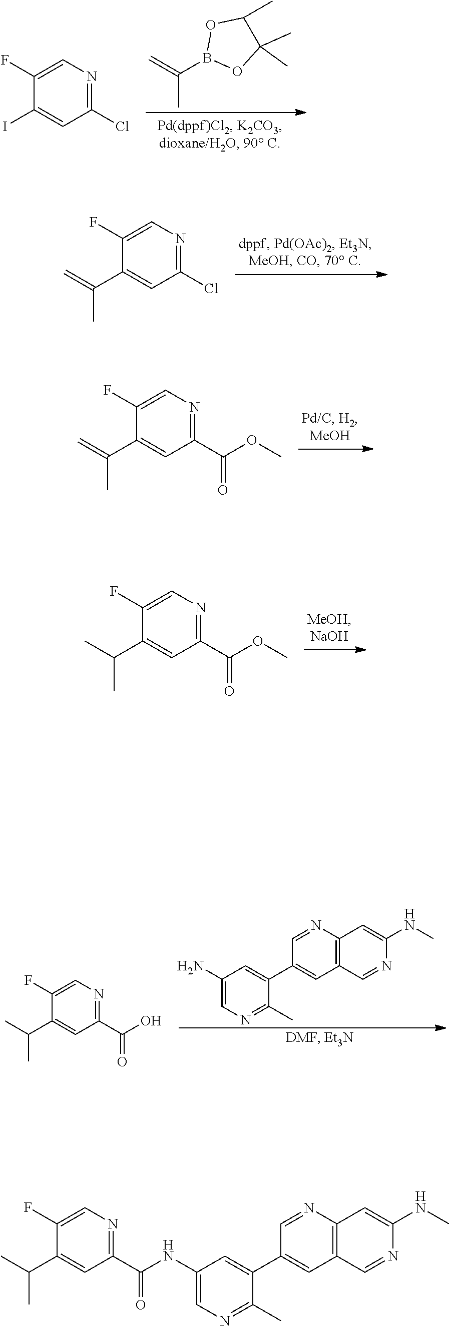

Step 1. A solution of 2-chloro-5-fluoro-4-iodopyridine (500 mg, 1.95 mmol), 4,4,5-trimethyl-2-(prop-1-en-2-yl)-1,3,2-dioxaborolane (300 mg, 1.95 mmol), [1,1′-bis(diphenylphosphino)ferrocene]dichloropalladium(II) (139 mg, 0.20 mmol), and potassium carbonate (538 mg, 3.90 mmol) in 1,4-dioxane (5 mL) and water (1 mL) was stirred at 90° C. for 3 hours under nitrogen. The mixture was diluted with water (10 mL) and extracted with ethyl acetate (15 mL×3). The combined organic layers were washed with brine (10 mL), dried over anhydrous sodium sulfate, filtered, and concentrated. The residue was purified by flash chromatography (silica, petroleum ether/ethyl acetate=20/1) to give 2-chloro-5-fluoro-4-(prop-1-en-2-yl)pyridine (298 mg, 1.74 mmol, 89.4%) as a yellow solid. MS (ESI) m/z 172.1 [M+H]⁺

Step 2. A mixture of 2-chloro-5-fluoro-4-(prop-1-en-2-yl)pyridine (298 mg, 1.74 mmol), palladium(II)acetate (84 mg, 0.17 mmol), 1,1′-bis(diphenylphosphino)ferrocene (185 mg, 0.35 mmol) and triethylamine (0.5 mL) in methanol (6 mL) was stirred at 70° C. for 16 hours under the protection of carbon monoxide. After cooling to room temperature, the reaction mixture was diluted with water (20 mL) and extracted with ethyl acetate (15 mL×3). The combined organic phases were washed brine (20 mL×2), dried over sodium sulfate, filtered, and concentrated. The residue was purified by flash chromatography (silica, petroleum ether/ethyl acetate=2/1) to give methyl 5-fluoro-4-(prop-1-en-2-yl)picolinate (87 mg, 0.45 mmol, 25.6%) as a yellow solid. MS (ESI) m/z 195.9 [M+H]⁺

Step 3. A mixture of methyl 5-fluoro-4-(prop-1-en-2-yl)picolinate (87 mg, 0.45 mmol) and palladium 10% on carbon (10 mg) in methanol (5 mL) was stirred at room temperature for 2 hours. The reaction mixture was diluted with water (10 mL) and extracted with ethyl acetate (10 mL×3). The combined organic phases were washed brine (15 mL×2), dried over sodium sulfate, filtered, and concentrated. The residue was purified by flash chromatography (silica, petroleum ether/ethyl acetate=1/1) to give methyl 5-fluoro-4-isopropylpicolinate (75 mg, 0.38 mmol, 84.6%) as a yellow solid. MS (ESI) m/z 198.1 [M+H]⁺

Step 4. A solution of methyl 5-fluoro-4-isopropylpicolinate (75 mg, 0.38 mmol) and sodium hydroxide (38 mg, 0.95 mmol) in methanol (3 mL) was stirred at room temperature for 1 hour. The mixture was washed with brine and extracted with ethyl acetate (10 mL×3). The combined organic phases were dried over sodium sulfate, filtered, and concentrated under reduced pressure to give 5-fluoro-4-isopropylpicolinic acid (45 mg, 0.25 mmol, 26%) as a yellow solid. MS (ESI) m/z 184.2 [M+H]⁺

Step 5. A solution of 5-fluoro-4-isopropylpicolinic acid (45 mg, 0.25 mmol), 3-(5-amino-2-methylpyridin-3-yl)-N-methyl-1,6-naphthyridin-7-amine (50 mg, 0.19 mmol), 1-[bis(dimethylamino)methylene]-1H-1,2,3-triazolo[4,5-b]pyridinium 3-oxid hexafluorophosphate (217 mg, 0.57 mmol), and triethylamine (0.2 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %. 30%-70% in 15 min) to afford 5-fluoro-4-isopropyl-N-(6-methyl-5-(7-(methylamino)-1,6-naphthyridin-3-yl)pyridin-3-yl)picolinamide (12.5 mg, 0.03 mmol, 15.3%) as a yellow solid. ¹H NMR (500 MHz, DMSO-d₆) δ 10.90 (s, 1H), 9.04 (d, J=2.3 Hz, 1H), 8.99 (s, 1H), 8.86 (d, J=2.3 Hz, 1H), 8.65 (s, 1H), 8.31 (d, J=1.9 Hz, 1H), 8.28 (d, J=2.3 Hz, 1H), 8.16 (d, J=6.2 Hz, 1H), 6.96 (d, J=5.0 Hz, 1H), 6.64 (s, 1H), 3.30-3.26 (m, 1H), 2.88 (d, J=4.9 Hz, 3H), 2.49 (s, 3H), 1.29 (d, J=6.9 Hz, 6H). MS (ESI) m/z 431.0 [M+H]⁺

### Example 92. Synthesis of 4-(2-cyanopropan-2-yl)-N-(2-fluoro-4-methyl-3-(7-(methylamino)-1,6-naphthyridin-3-yl)phenyl)picolinamide (Compound 99)

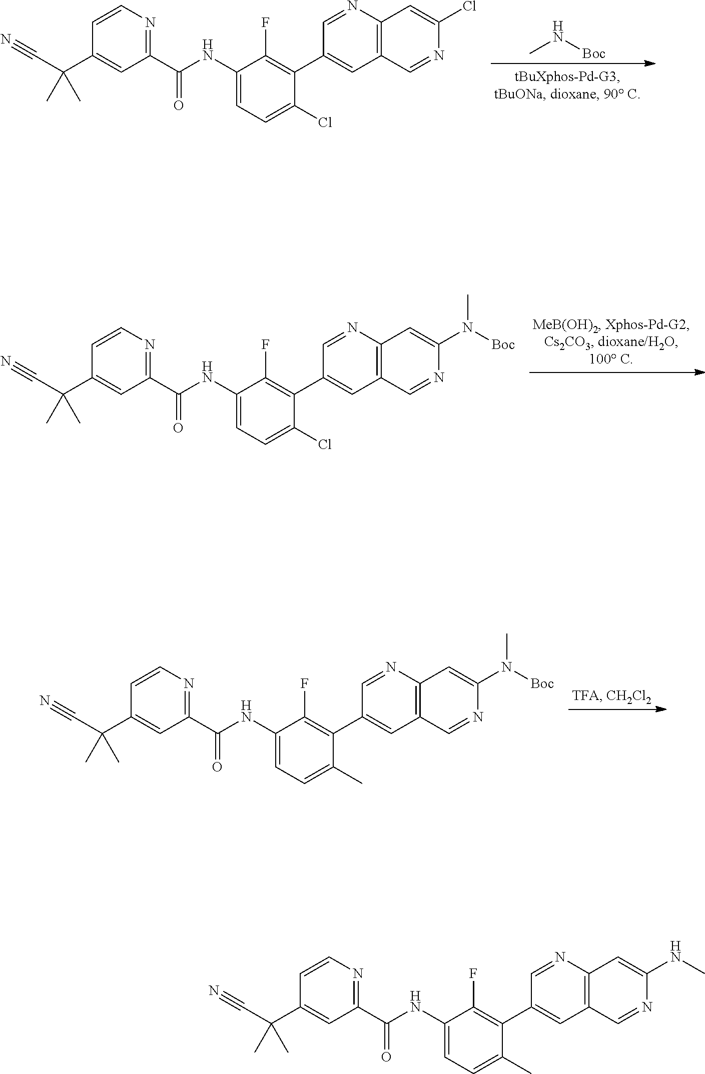

Step 1. To a solution of N-[4-chloro-3-(7-chloro-1,6-naphthyridin-3-yl)-2-fluoro-phenyl]-4-(1-cyano-1-methyl-ethyl)pyridine-2-carboxamide (390 mg, 811.96 μmol) and tert-butyl N-methylcarbamate (213.01 mg, 1.62 mmol) in dioxane (4 mL) was added [(2-di-tert-butylphosphino-2′,4′,6′-triisopropyl-1,1′-biphenyl)-2-(2′-amino-1,1′-biphenyl)] palladium(II) methanesulfonate (tBuXphos-Pd-G3, 64.50 mg, 81.20 μmol) and sodium tert-butoxide (2 M, 1.22 mL) under nitrogen atmosphere. The mixture solution was stirred at 90° C. under nitrogen atmosphere for 16 hours. On completion, the solution was concentrated and the residue was purified by reversed-phase HPLC (0.1% formic acid condition) to give tert-butyl(3-(6-chloro-3-(4-(2-cyanopropan-2-yl)picolinamido)-2-fluorophenyl)-1,6-naphthyridin-7-yl(methyl)carbamate (30 mg, 52.17 μmol, 6.4%) as a yellow solid. MS (ESI) m/z 575.1 [M+H]⁺

Step 2. To a solution of tert-butyl(3-(6-chloro-3-(4-(2-cyanopropan-2-yl)picolinamido)-2-fluorophenyl)-1,6-naphthyridin-7-yl)methyl)carbamate (30 mg, 52.2 μmol) and methylboronic acid (31.23 mg, 522 μmol) in dioxane (1 mL) and water (0.2 ML) was added Chloro(2-dicyclohexylphosphino-2′,4′,6′-triisopropyl-1,1′-biphenyl)[2-(2′-amino-1,1′-biphenyl)]palladium(II) (XPhos-Pd-G2, 4.10 mg, 5.22 μmol) and potassium carbonate (21.63 mg, 157 μmol). The mixture solution was stirred at 100° C. for 16 hours under nitrogen atmosphere. On completion, the solution was concentrated and the residue was purified by reversed-phase HPLC (0.1% formic acid condition) to give tert-butyl (3-(3-(4-(2-cyanopropan-2-yl)picolinamido)-2-fluoro-6-methylphenyl)-1,6-naphthyridin-7-yl)(methyl)carbamate (20 mg, 36.06 μmol, 69%) as a yellow solid. MS (ESI) m/z 555.2 [M+H]⁺

Step 3. A solution of tert-butyl (3-(3-(4-(2-cyanopropan-2-yl)picolinamido)-2-fluoro-6-methylphenyl)-1,6-naphthyridin-7-yl)(methyl)carbamate (20 mg, 36.06 μmol) in dichloromethane (1 mL) and trifluoroacetic acid (0.2 mL, 2.70 mmol) was stirred at 25° C. for 15 minutes. On completion, the solution was concentrated. The residue was purified by prep-HPLC (Column: Phenomenex Luna C18 150×25 mm, 5 μm; Mobile phase: water (formic acid)-acetonitrile; B %: 29%-59%, 10 min) to give 4-(2-cyanopropan-2-yl)-N-(2-fluoro-4-methyl-3-(7-(methylamino)-1,6-naphthyridin-3-yl)phenyl)picolinamide (11.32 mg, 24.64 μmol, 68%) as a yellow solid. ¹H NMR (400 MHz, CD₃OD) δ=8.96 (s, 1H), 8.76-8.74 (m, 2H), 8.42 (d, J=2.0 Hz, 1H), 8.29-8.25 (m, 2H), 7.82-7.81 (m, 1H), 7.28 (d, J=7.6 Hz, 1H), 6.75 (s, 1H), 3.01 (s, 3H), 2.28 (s, 3H), 1.83 (s, 6H). MS (ESI) m/z 455.0 [M+H]⁺

### Example 93. Synthesis of 3-(difluoromethoxy)-2-(difluoromethyl)-N-(6-methyl-5-(7-(methylamino)-1,6-naphthyridin-3-yl)pyridin-3-yl)isonicotinamide (Compound 100)

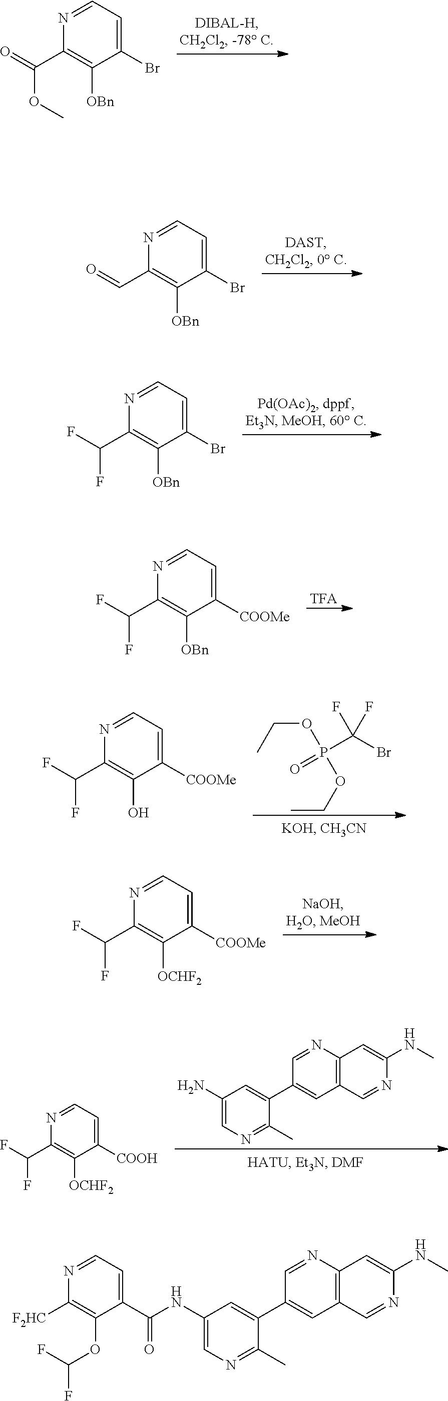

Step 1. To a solution of methyl 3-(benzyloxy)-4-bromopicolinate (1.8 g, 5.6 mmol) in dichloromethane (20 mL) was added diisobutylaluminium hydride (6.2 mL, 6.2 mmol, 1 M in cyclohexane) at −78° C. The mixture was stirred at −78° C. for 1 hour. The reaction mixture was quenched by sodium sulfate decahydrate (5 g) and stirred at room temperature for 1 hour. The mixture was filtered, washed with ethyl acetate (30 mL). The filtrate was concentrated to give 3-(benzyloxy)-4-bromopicolinaldehyde (1.2 g, 4.1 mmol, 73%) as a yellow oil. MS (ESI) m/z 292.0 [M+H]⁺

Step 2. To a solution of 3-(benzyloxy)-4-bromopicolinaldehyde (1.2 g, 4.12 mmol) in dichloromethane (30 mL) was added diethylaminosulfur trifluoride (1.66 g, 10.31 mmol) at 0° C. The mixture was stirred at 0° C. for 2 hours. The reaction mixture was quenched with water (100 mL) and extracted with dichloromethane (100 mL×2). The combined organic phases were concentrated and purified by flash chromatography (silica, petroleum ether/ethyl acetate=4/1) to give 3-(benzyloxy)-4-bromo-2-(difluoromethyl)pyridine (450 mg, 1.44 mmol, 35%) as a yellow oil. MS (ESI) m/z 314.0 [M+H]⁺

Step 3. A mixture of 3-(benzyloxy)-4-bromo-2-(difluoromethyl)pyridine (500 mg, 1.6 mmol), 1,1′-bis(diphenyl phosphino)ferrocene (177 mg, 0.32 mmol), palladium (II) acetate (36 mg, 0.16 mmol), and triethylamine (484 mg, 4.8 mmol) in methanol (20 mL) was stirred at 60° C. for 18 hours under carbon monoxide atmosphere. The reaction was cooled to room temperature and concentrated. The residue was purified by flash chromatography (silica, petroleum ether/ethyl acetate=4/1) to give methyl 3-(benzyloxy)-2-(difluoromethyl)isonicotinate (320 mg, 1.09 mmol, 68%) as a yellow oil. MS (EST) m/z 294.0 [M+H]⁺

Step 4. A solution of methyl 3-(benzyloxy)-2-(difluoromethyl)isonicotinate (320 mg, 1.09 mmol) in trifluoroacetic acid (2 mL) was stirred at room temperature for 5 hours. The reaction mixture was concentrated. The residue was purified by flash chromatography (silica, petroleum ether/ethyl acetate=1/1) to give methyl 2-(difluoromethyl)-3-hydroxyisonicotinate (180 mg, 0.89 mmol, 81%) as a yellow solid. MS (ESI) m/z 204.1 [M+H]⁺

Step 5. To a solution of methyl 2-(difluoromethyl)-3-hydroxyisonicotinate (180 mg, 0.89 mmol) and potassium hydroxide (248 mg, 4.43 mmol) in acetonitrile (13 mL) was added diethyl (bromodifluoromethyl)phosphonate (473 mg, 1.77 mmol). The mixture was stirred at room temperature for 2 hours. The reaction mixture was filtered, washed with acetonitrile (3 mL). The filtrate was concentrated. The residue was purified by flash chromatography (silica, petroleum ether/ethyl acetate=3/1) to give methyl 3-(difluoromethoxy)-2-(difluoromethyl)isonicotinate (80 mg, 0.32 mmol, 36%) as a yellow oil. MS (ESI) m/z 254.1 [M+H]⁺

Step 6. A mixture of methyl 3-(difluoromethoxy)-2-(difluoromethyl)isonicotinate (80 mg, 0.32 mmol) and sodium hydroxide (0.32 mL, 0.63 mmol, 2 M in water) in methanol (1 mL) was stirred at room temperature for 2 hours. The reaction mixture was treated with water (10 mL), and extracted with ethyl acetate (10 mL). The aqueous phase was separated and its pH was adjusted to 4 with hydrochloric acid (1 N), then extracted with ethyl acetate (10 mL×2). The combined organic phases were concentrated to give 3-(difluoromethoxy)-2-(difluoromethyl)isonicotinic acid (40 mg, 0.17 mmol, 52%) as a yellow oil. MS (ESI) m/z 240.0 [M+H]⁺

Step 7. A mixture of 3-(difluoromethoxy)-2-(difluoromethyl)isonicotinic acid (40 mg, 0.17 mmol), 3-(5-amino-2-methylpyridin-3-yl)-N-methyl-1,6-naphthyridin-7-amine (44 mg, 0.17 mmol), 1-[bis(dimethylamino)methylene]-1H-1,2,3-triazolo[4,5-b]pyridinium 3-oxid hexafluorophosphate (76 mg, 0.2 mmol), and triethylamine (34 mg, 0.33 mmol) in N,N-dimethylformamide (2 mL) was stirred at room temperature for 2 hours. The reaction mixture was treated with water (30 mL), and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-(difluoromethoxy)-2-(difluoromethyl)-N-(6-methyl-5-(7-(methylamino)-1,6-naphthyridin-3-yl)pyridin-3-yl)isonicotinamide (12.2 mg, 0.025 mmol, 15%) as a yellow solid. ¹H NMR (500 MHz, DMSO-d₆) δ 10.99 (s, 1H), 9.00 (s, 1H), 8.88-8.80 (m, 2H), 8.75 (d, J=2.4 Hz, 1H), 8.31 (d, J=2.1 Hz, 1H), 8.07 (d, J=2.4 Hz, 1H), 7.96 (d, J=4.8 Hz, 1H), 7.37-7.00 (m, 2H), 6.96 (q, J=4.9 Hz, 1H), 6.63 (s, 1H), 2.87 (d, J=5.0 Hz, 3H), 2.49 (s, 3H). MS (EST) m/z 487.0 [M+H]⁺

### Example 94. Synthesis of 4-(2-(difluoromethoxy)propan-2-yl)-N-(4-methyl-3-(7-(methylamino)-1,6-naphthyridin-3-yl)phenyl)picolinamide (Compound 101)

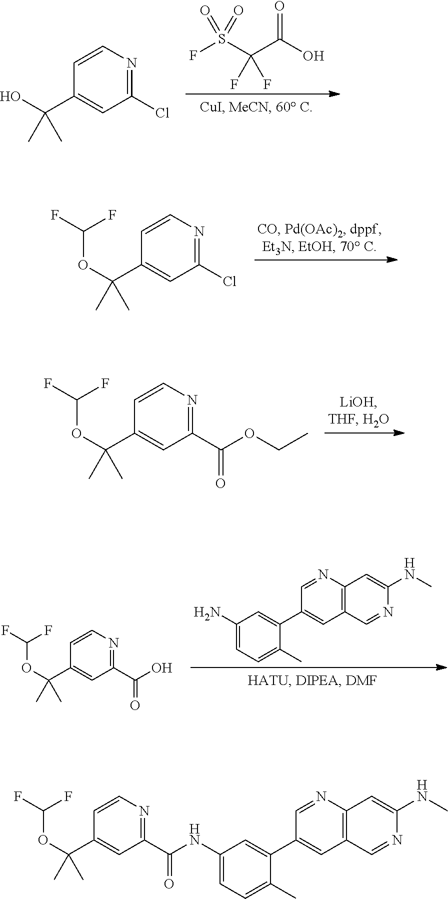

Step 1. A solution of 2-(2-chloropyridin-4-yl)propan-2-ol (500 mg, 2.91 mmol) and copper(I) iodide (110 mg, 0.58 mmol) in acetonitrile (20 mL) was stirred at 60° C. for 10 minutes and 2,2-difluoro-2-(fluorosulfonyl)acetic acid (1.81 g, 10.19 mmol) was added. The reaction was stirred at 60° C. for 0.5 h under argon. After cooling to room temperature, the reaction solution was quenched with ice water (60 mL) and extracted with ethyl acetate (50 mL×2). The combined organic phases were concentrated and purified by flash chromatography (silica, petroleum ether/ethyl acetate=1/1) to give 2-chloro-4-(2-(difluoromethoxy)propan-2-yl)pyridine (185 mg, 0.83 mmol, 29%) as a yellow oil. MS (ESI) m/z 172.1 [M+H]⁺

Step 2. A mixture of 2-chloro-4-(2-(difluoromethoxy)propan-2-yl)pyridine (185 mg, 0.83 mmol), 1,1′-bis(diphenyl phosphino)ferrocene (184 mg, 0.332 mmol), palladium (II) acetate (37 mg, 0.166 mmol) and triethylamine (336 mg, 3.32 mmol) in ethanol (10 mL) was stirred at 70° C. for 16 hours under carbon monoxide atmosphere. The reaction was cooled to room temperature and concentrated. The residue was purified by flash chromatography (silica, petroleum ether/ethyl acetate=100/90) to give ethyl 4-(2-(difluoromethoxy)propan-2-yl)picolinate (135 mg, 0.87 mmol, 52%) as a brown oil. MS (ESI) m/z 260.1 [M+H]⁺

Step 3. To a solution of ethyl 4-(2-(difluoromethoxy)propan-2-yl)picolinate (150 mg, 0.58 mmol) in tetrahydrofuran (1.5 mL) were added lithium hydroxide hydrate (122 mg, 2.9 mmol) and water (1.5 mL) at 25° C. After stirring at 25° C. for 2 hours, the solvent was evaporated under reduced pressure. The residue was diluted with water (10 mL), adjusted to pH 2 by adding 1 N hydrochloric acid slowly and extracted with ethyl acetate (30 mL×3). The combined organic layers were dried over anhydrous sodium sulfate, filtered, and concentrated to give 4-(2-(difluoromethoxy)propan-2-yl)picolinic acid (90 mg, 0.39 mmol, 67%) as a yellow solid. MS (ESI) m/z 232.1 [M+H]⁺

Step 4. A solution of 3-(5-amino-2-methylphenyl)-N-methyl-1,6-naphthyridin-7-amine (32 mg, 0.12 mmol), 4-(2-(difluoromethoxy)propan-2-yl)picolinic acid (33 mg, 0.14 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (62 mg, 0.48 mmol) in N,N-dimethylformamide (3 mL) was stirred at 25° C. for 3 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %. 30%-70% in 15 min) to afford 4-(2-(difluoromethoxy)propan-2-yl)-N-(4-methyl-3-(7-(methylamino)-1,6-naphthyridin-3-yl)phenyl)picolinamide (18.9 mg, 0.04 mmol, 33%) as a yellow solid. ¹H NMR (400 MHz, DMSO-d₆) δ 10.74 (s, 1H), 8.98 (s, 1H), 8.83 (d, J=2.3 Hz, 1H), 8.76 (d, J=5.1 Hz, 1H), 8.25 (d, J=1.9 Hz, 1H), 8.20 (d, J=1.3 Hz, 1H), 7.94 (d, J=2.2 Hz, 1H), 7.90 (dd, J=8.2, 2.2 Hz, 1H), 7.73 (dd, J=5.2, 1.9 Hz, 1H), 7.36 (d, J=8.5 Hz, 1H), 7.03-6.59 (m, 3H), 2.87 (d, J=5.0 Hz, 3H), 2.31 (d, J=12.9 Hz, 3H), 1.70 (s, 6H). MS (ESI) m/z 478.1 [M+H]⁺

### Example 95. Synthesis of 5-(2-fluoropropan-2-yl)-N-(6-methyl-5-(7-(methylamino)-1,6-naphthyridin-3-yl)pyridin-3-yl)nicotinamide (Compound 102)

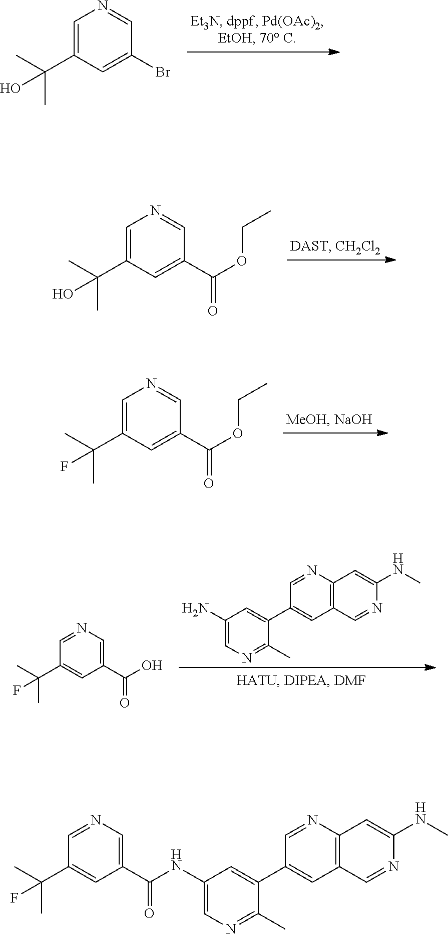 [SMILES: CC(C)(O)c1cncc(Br)c1 ; CCOC(=O)c1cncc(C(C)(C)O)c1 ; CCOC(=O)c1cncc(C(C)(C)F)c1 ; CC(C)(F)c1cncc(C(=O)O)c1 ; CNc1cc2ncc(-c3cc(N)cnc3C)cc2cn1 ; CNc1cc2ncc(-c3cc(NC(=O)c4cncc(C(C)(C)F)c4)cnc3C)cc2cn1]

Step 1. A mixture of 2-(5-bromopyridin-3-yl)propan-2-ol (300 mg, 1.39 mmol), palladium(II) acetate (32 mg, 0.14 mmol), 1,1′-bis(diphenylphosphino)ferrocene (155 mg, 0.28 mmol), and triethylamine (421 mg, 4.17 mmol) in ethanol (6 mL) was stirred at 70° C. for 16 hours under carbon monoxide atmosphere. After cooling to room temperature, the reaction mixture was diluted with water (100 mL) and extracted with ethyl acetate (100 mL×3). The combined organic phases were washed brine (100 mL×2), dried over sodium sulfate, filtered, and concentrated. The residue was purified by flash chromatography (silica, petroleum ether/ethyl acetate=1/2) to give ethyl 5-(2-hydroxypropan-2-yl)nicotinate (250 mg, 1.19 mmol, 86.6%) as a yellow solid. MS (ESI) m/z 210.1 [M+H]⁺

Step 2. A mixture of ethyl 5-(2-hydroxypropan-2-yl)nicotinate (250 mg, 1.19 mmol) and diethylaminosulphur trifluoride (958 mg, 5.95 mmol) in dichloromethane (5 mL) was stirred at room temperature for 16 hours. The reaction mixture was diluted with water (50 mL) and extracted with ethyl acetate (50 mL×3). The combined organic phases were washed brine (50 mL×2), dried over sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give ethyl 5-(2-fluoropropan-2-yl)nicotinate (200 mg, 0.95 mmol, 80%) as a yellow solid. MS (EST) m/z 212.2 [M+H]⁺

Step 3. A solution of ethyl 5-(2-fluoropropan-2-yl)nicotinate (200 mg, 0.95 mmol) and sodium hydroxide (38 mg, 0.95 mmol) in methanol (3 mL) was stirred at room temperature for 2 hours. The solvent was evaporated under reduced pressure, and the residue was diluted with water (5 mL). The mixture was adjusted to pH=3 by adding 1 N hydrochloric acid slowly and extracted with ethyl acetate (50 mL×3). The combined organic phases were dried over sodium sulfate, filtered, and concentrated under reduced pressure to give 5-(2-fluoropropan-2-yl)nicotinic acid (80 mg, 0.43 mmol, 96%) as a yellow solid. MS (ESI) m/z 184.2 [M+H]⁺

Step 4. A solution of 5-(2-fluoropropan-2-yl)nicotinic acid (30 mg, 0.16 mmol), 3-(5-amino-2-methylpyridin-3-yl)-N-methyl-1,6-naphthyridin-7-amine (43 mg, 0.16 mmol), 1-[bis(dimethylamino)methylene]-1H-1,2,3-triazolo[4,5-b]pyridinium 3-oxid hexafluorophosphate (91 mg, 0.24 mmol), and N,N-diisopropylethylamine (62 mg, 0.48 mmol) in N,N-dimethylformamide (3 mL) was stirred at room temperature for 2 hours. The mixture was quenched with brine (15 mL) and extracted with ethyl acetate (10 mL×3). The combined organic phases were dried over sodium sulfate, filtered, and concentrated to give 5-(2-fluoropropan-2-yl)-N-(6-methyl-5-(7-(methylamino)-1,6-naphthyridin-3-yl)pyridin-3-yl)nicotinamide (27.5 mg, 0.064 mmol, 40%) as a yellow solid. ¹H NMR (500 MHz, DMSO-d₆) δ 10.73 (s, 1H), 9.10 (d, J=2.0 Hz, 1H), 9.00 (s, 1H), 8.88 (dd, J=14.8, 2.3 Hz, 3H), 8.34 (dd, J=15.7, 2.0 Hz, 2H), 8.16 (d, J=2.3 Hz, 1H), 6.97 (d, J=5.0 Hz, 1H), 6.64 (s, 1H), 2.88 (d, J=4.8 Hz, 3H), 2.51 (s, 3H), 1.78 (s, 3H), 1.74 (s, 3H). MS (ESI) m/z 431.1 [M+H]⁺

### Example 96. Synthesis of 4-isopropyl-3-methoxy-N-(6-methyl-5-(7-(methylamino)-1,6-naphthyridin-3-yl)pyridin-3-yl)picolinamide (Compound 103)

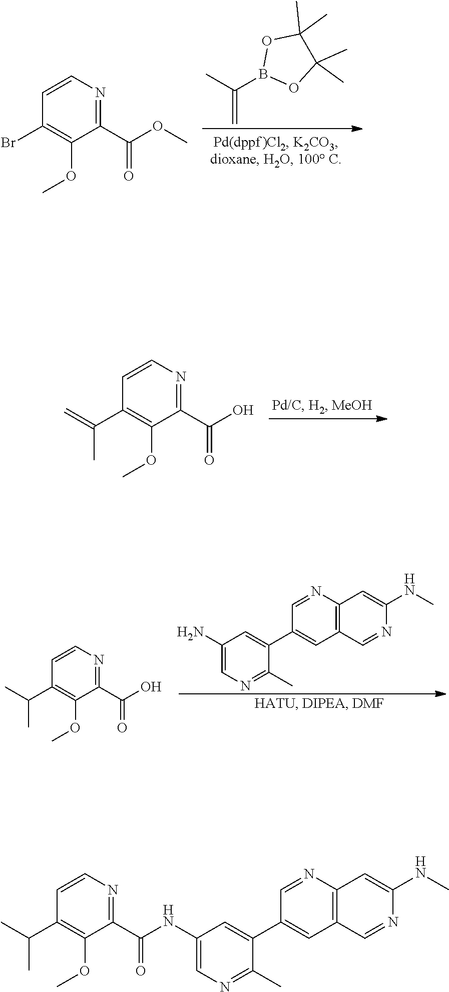 [SMILES: COC(=O)c1nccc(Br)c1OC ; C=C(C)B1OC(C)(C)C(C)(C)O1 ; C=C(C)c1ccnc(C(=O)O)c1OC ; COc1c(C(C)C)ccnc1C(=O)O ; CNc1cc2ncc(-c3cc(N)cnc3C)cc2cn1 ; CNc1cc2ncc(-c3cc(NC(=O)c4nccc(C(C)C)c4OC)cnc3C)cc2cn1]

Step 1. A solution of methyl 4-bromo-3-methoxypicolinate (500 mg, 2.0 mmol), [1,1′-bis(diphenylphosphino)ferrocene]dichloropalladium(II) (146.2 mg, 0.2 mmol), 4,4,5,5-tetramethyl-2-(prop-1-en-2-yl)-1,3,2-dioxaborolane (403.2 mg, 2.4 mmol), and potassium carbonate (552 mg, 4.0 mmol) in water (2 mL) and 1,4-dioxane (10 mL) was stirred at 100° C. for 16 hours under nitrogen. After cooling to room temperature, the mixture was adjusted to pH=3 by slow addition of 1 N hydrochloric acid. The mixture was concentrated and the residue was purified by flash chromatography (silica, petroleum ether/ethyl acetate=1/5) to give 3-methoxy-4-(prop-1-en-2-yl)picolinic acid (370 mg, crude) as a yellow oil. MS (ESI) m/z 194.1 [M+H]⁺

Step 2. A mixture of 3-methoxy-4-(prop-1-en-2-yl)picolinic acid (270 mg, crude) and palladium (10% on carbon, 300 mg) in methanol (10 mL) was stirred at room temperature for 16 hours under hydrogen. The reaction mixture was filtered through celite and concentrated to give 4-isopropyl-3-methoxypicolinic acid (46 mg, 0.24 mmol) as a yellow oil. The crude product was used without further purification. MS (ESI) m/z 196.2 [M+H]⁺

Step 3. A solution of 4-isopropyl-3-methoxypicolinic acid (23 mg, 0.12 mmol), 3-(5-amino-2-methylpyridin-3-yl)-N-methyl-1,6-naphthyridin-7-amine (26 mg, 0.1 mmol), 1-[bis(dimethylamino)methylene]-1H-1,2,3-triazolo[4,5-b]pyridinium 3-oxid hexafluorophosphate (91 mg, 0.24 mmol), and N,N-diisopropylethylamine (46.4 mg, 0.36 mmol) in N,N-dimethylformamide (3 mL) was stirred at room temperature for 4 hours. The reaction solution was diluted with ethyl acetate (50 mL), washed with water (20 mL×2) and brine (20 mL2), dried over sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give 4-isopropyl-3-methoxy-N-(6-methyl-5-(7-(methylamino)-1,6-naphthyridin-3-yl)pyridin-3-yl)picolinamide (21.5 mg, 0.049 mmol) as a yellow solid. ¹H NMR (500 MHz, DMSO-d₆) δ 10.55 (s, 1H), 8.99 (s, 1H), 8.86 (s, 2H), 8.32 (s, 1H), 8.17 (d, J=2.1 Hz, 1H), 7.60 (d, J=8.7 Hz, 1H), 7.44 (d, J=8.7 Hz, 1H), 6.95 (d, J=5.0 Hz, 1H), 6.63 (s, 1H), 3.87-3.73 (m, 3H), 3.11-2.96 (m, 1H), 2.87 (d, J=4.9 Hz, 3H), 2.48 (s, 3H), 1.23 (t, J=14.4 Hz, 6H). MS (ESI) m/z 443.0 [M+H]⁺

### Example 97. Synthesis of 3-fluoro-2-isopropyl-N-(6-methyl-5-(7-(methylamino)-1,6-naphthyridin-3-yl)pyridin-3-yl)isonicotinamide (Compound 104)

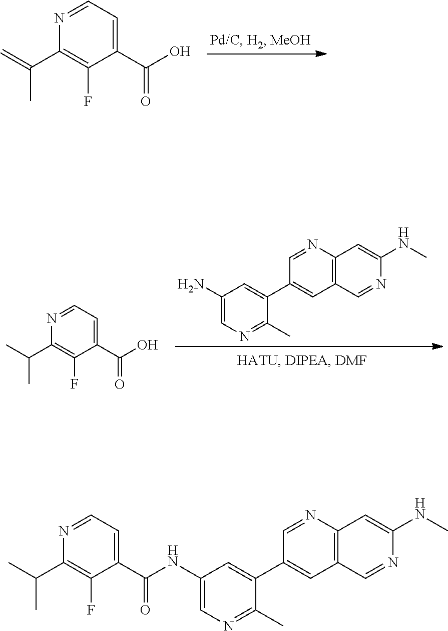 [SMILES: C=C(C)c1nccc(C(=O)O)c1F ; CC(C)c1nccc(C(=O)O)c1F ; CNc1cc2ncc(-c3cc(N)cnc3C)cc2cn1 ; CNc1cc2ncc(-c3cc(NC(=O)c4ccnc(C(C)C)c4F)cnc3C)cc2cn1]

Step 1. A mixture of 3-fluoro-2-(prop-1-en-2-yl)isonicotinic acid (50 mg, 0.28 mmol) and palladium (10% on activated carbon, 5 mg) in methanol (5 mL) was stirred at room temperature for 2 hours under hydrogen. The mixture was filtered through celite and the filtrate was concentrated. The mixture was purified by prep-HPLC (Column: Xbridge 21.2×250 mm C18, 10 μm, Mobile Phase A: water (10 mM ammonium bicarbonate), B: acetonitrile) to give 3-fluoro-2-isopropylisonicotinic acid (9 mg, 0.05 mmol, 18%) as a white solid. MS (ESI) m/z 184.2 [M+H]⁺

Step 2. A mixture of 3-fluoro-2-isopropylisonicotinic acid (9 mg, 0.05 mmol), 3-(5-amino-2-methylpyridin-3-yl)-N-methyl-1,6-naphthyridin-7-amine (13 mg, 0.05 mmol), 1-[bis(dimethylamino)methylene]-1H-1,2,3-triazolo[4,5-b]pyridinium 3-oxid hexafluorophosphate (22 mg, 0.06 mmol), and N,N-diisopropylethylamine (13 mg, 0.1 mmol) in N,N-dimethylformamide (3 mL) was stirred at room temperature for 4 hours. The mixture was purified by prep-HPLC (Column: Xbridge 21.2×250 mm C18, 10 μm, Mobile Phase A: water (10 mM ammonium bicarbonate), B: acetonitrile) to give 3-fluoro-2-isopropyl-N-(6-methyl-5-(7-(methylamino)-1,6-naphthyridin-3-yl)pyridin-3-yl)isonicotinamide (9 mg, 0.02 mmol, 41%) as a yellow solid. ¹H NMR (500 MHz, DMSO-d₆) δ 10.93 (s, 1H), 9.00 (s, 1H), 8.86 (d, J=2.5 Hz, 1H), 8.79 (d, J=2.5 Hz, 1H), 8.83 (d, J=4.5 Hz, 1H), 8.32 (d, J=1.6 Hz, 1H), 8.12 (d, J=2.5 Hz, 1H), 7.55 (t, J=5.0 Hz, 1H), 6.97 (q, J=5.0 Hz, 1H), 6.64 (s, 1H), 3.45-3.38 (m, 1H), 2.88 (d, J=4.5 Hz, 3H), 2.49 (s, 3H), 1.28 (d, J=6.5 Hz, 6H). MS (ESI) m/z 431.1 [M+H]⁺

### Example 98. Synthesis of (S)—N-(6-methyl-5-(7-(methylamino)-1,6-naphthyridin-3-yl)pyridin-3-yl)-5-(2,2,2-trifluoro-1-hydroxyethyl)nicotinamide and (R)—N-(6-methyl-5-(7-(methylamino)-1,6-naphthyridin-3-yl)pyridin-3-yl)-5-(2,2,2-trifluoro-1-hydroxyethyl)nicotinamide (Compounds 105 and 111)

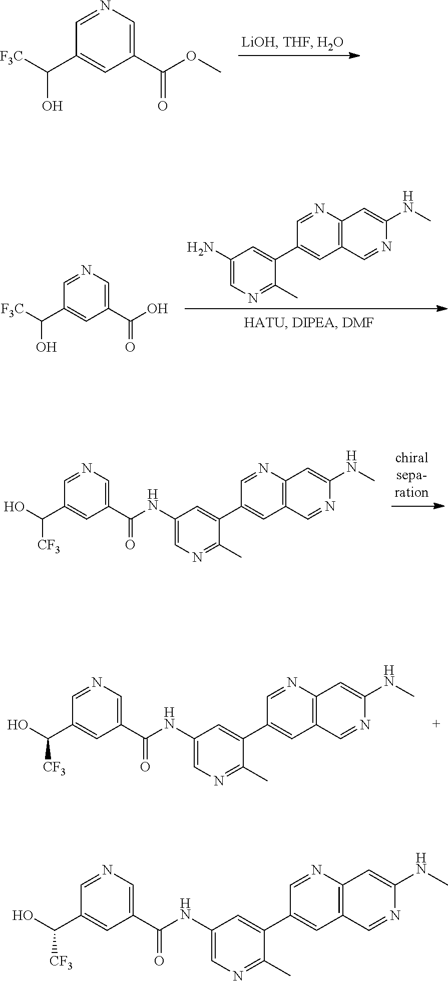 [SMILES: COC(=O)c1cncc(C(O)C(F)(F)F)c1 ; O=C(O)c1cncc(C(O)C(F)(F)F)c1 ; CNc1cc2ncc(-c3cc(N)cnc3C)cc2cn1 ; CNc1cc2ncc(-c3cc(NC(=O)c4cncc(C(O)C(F)(F)F)c4)cnc3C)cc2cn1 ; CNc1cc2ncc(-c3cc(NC(=O)c4cncc([C@H](O)C(F)(F)F)c4)cnc3C)cc2cn1 ; CNc1cc2ncc(-c3cc(NC(=O)c4cncc([C@@H](O)C(F)(F)F)c4)cnc3C)cc2cn1]

Step 1. To a solution of methyl 5-(2,2,2-trifluoro-1-hydroxyethyl)nicotinate (532 mg, 2.26 mmol) in tetrahydrofuran (2 mL) were added lithium hydroxide monohydrate (190 mg, 4.52 mmol) and water (2 mL). After stirring at 25° C. for 2 hours, the solvent was evaporated under reduced pressure. The residue was diluted with water (10 mL) and adjusted to pH=3 by adding 1 N hydrochloric acid slowly. The resulting white precipitate was collected by filtration, washed with water (5 mL) and dried to give 5-(2,2,2-trifluoro-1-hydroxyethyl)nicotinic acid (420 mg, 3.56 mmol, 91%) as a yellow solid. MS (ESI) m/z 222.1 [M+H]⁺

Step 2. A solution of 3-(5-amino-2-methylpyridin-3-yl)-N-methyl-1,6-naphthyridin-7-amine (69 mg, 0.26 mmol), 5-(2,2,2-trifluoro-1-hydroxyethyl)nicotinic acid (86 mg, 0.39 mmol), 1-[bis(dimethylamino)methylene]-1H-1,2,3-triazolo[4,5-b]pyridinium 3-oxid hexafluorophosphate (198 mg, 0.52 mmol), and N,N-diisopropylethylamine (134 mg, 1.04 mmol) in N,N-dimethylformamide (3 mL) was stirred at 25° C. for 18 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(6-methyl-5-(7-(methylamino)-1,6-naphthyridin-3-yl)pyridin-3-yl)-5-(2,2,2-trifluoro-1-hydroxyethyl)nicotinamide (40 mg, 0.085 mmol, 33%) as a yellow solid. MS (ESI) m/z 469.1 [M+H]⁺

Step 3. The enantiomers of N-(6-methyl-5-(7-(methylamino)-1,6-naphthyridin-3-yl)pyridin-3-yl)-5-(2,2,2-trifluoro-1-hydroxyethyl)nicotinamide (40 mg, 0.085 mmol) were separated by chiral SFC (Instrument: SFC-150 (Waters); Column: AS 20×250 mm, 10 μm (Daicel); Mobile phase: CO₂/MeOH (0.2% methanol ammonia)=70/30; Detection wavelength: 214 nm) to afford the first eluting enantiomer (retention time 3.3 min) which was arbitrarily assigned as (S)—N-(6-methyl-5-(7-(methylamino)-1,6-naphthyridin-3-yl)pyridin-3-yl)-5-(2,2,2-trifluoro-1-hydroxyethyl)nicotinamide (8.4 mg, 0.018 mmol) as a yellow solid. ¹H NMR (400 MHz, DMSO-d₆) δ 10.80 (s, 1H), 9.20 (d, 1=2.1 Hz, 1H), 9.00 (s, 1H), 8.92-8.84 (m, 3H), 8.46 (s, 1H), 8.32 (d, J=2.0 Hz, 1H), 8.17 (d, J=2.3 Hz, 1H), 7.26 (d, J=5.4 Hz, 1H), 6.97 (q, J=5.0 Hz, 1H), 6.64 (s, 1H), 5.53-5.43 (m, 1H), 2.88 (d, J=5.0 Hz, 3H), 2.50 (s, 3H). MS (ESI) m/z 469.0 [M+H]⁺;
and the second eluting enantiomer (retention time 3.6 min) which was arbitrarily assigned as (R)—N-(6-methyl-5-(7-(methylamino)-1,6-naphthyridin-3-yl)pyridin-3-yl)-5-(2,2,2-trifluoro-1-hydroxyethyl)nicotinamide (5.3 mg, 0.011 mmol) as a yellow solid. ¹H NMR (400 MHz, DMSO-d₆) δ 10.80 (s, 1H), 9.20 (d, J=2.1 Hz, 1H), 9.00 (s, 1H), 8.91-8.85 (m, 3H), 8.46 (s, 1H), 8.32 (d, J=2.2 Hz, 1H), 8.16 (d, J=2.4 Hz, 1H), 7.26 (d, J=5.8 Hz, 1H), 6.97 (q, J=5.0 Hz, 1H), 6.64 (s, 1H), 5.53-5.43 (m, 1H), 2.87 (d, J=5.0 Hz, 3H), 2.51 (s, 3H). MS (ESI) m/z 469.0 [M+H]⁺

### Example 99. Synthesis of 3,3-dimethyl-N-(6-methyl-5-(7-(methylamino)-1,6-naphthyridin-3-yl)pyridin-3-yl)-2,3-dihydrofuro[2,3-c]pyridine-7-carboxamide (Compound 106)

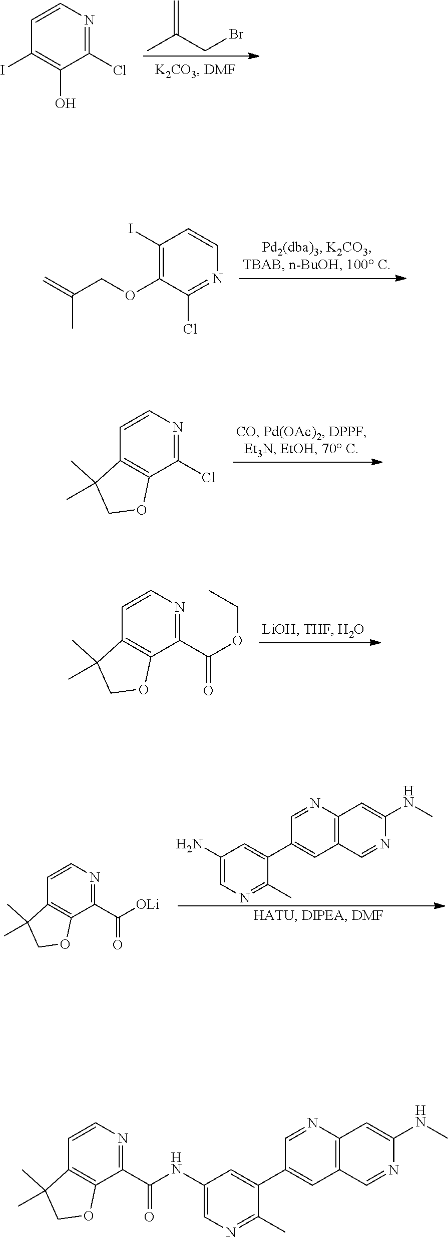

Step 1. The mixture of 2-chloro-4-iodopyridin-3-ol (1.02 g, 4.0 mmol), 3-bromo-2-methylprop-1-ene (648 mg, 4.8 mmol) and potassium carbonate (1.66 g, 12 mmol) in N,N-dimethylformamide (15 mL) was stirred at 25° C. for 3 hour. The mixture was diluted with ethyl acetate (100 mL) and washed with water (100 mL). The organic phase was washed with brine (40 mL×3), dried over sodium sulfate, filtered, and concentrated. The residue was purified by flash chromatography (silica, ethyl acetate/petroleum ether=18/100) to get 2-chloro-4-iodo-3-(2-methylallyloxy)pyridine (1.1 g, 3.55 mmol, 89%) as a white solid. MS (ESI) m/z 310.0 [M+H]⁺

Step 2. A mixture of 2-chloro-4-iodo-3-(2-methylallyloxy)pyridine (500 mg, 1.62 mmol), tris(dibenzylideneacetone)dipalladium(0) (148 mg, 0.162 mmol), potassium carbonate (647 mg, 4.68 mmol) and tetrabutylammonium bromide (52 mg, 0.162 mmol) in n-butanol (10 mL) was heated to 100° C. and stirred for 16 hours. The reaction was diluted with water (100 mL) and extracted with ethyl acetate (50 mL×2). The combined organic phases were washed with brine (50 mL), dried over sodium sulfate, filtered, and concentrated. The crude residue was purified by flash chromatography (silica, ethyl acetate/petroleum ether=18/100) to give 7-chloro-3,3-dimethyl-2,3-dihydrofuro[2,3-c]pyridine (120 mg, 0.65 mmol, 40%) as a yellow oil. MS (ESI) m/z 184.2 [M+H]⁺

Step 3. A mixture of 7-chloro-3,3-dimethyl-2,3-dihydrofuro[2,3-c]pyridine (120 mg, 0.65 mmol), 1,1′-bis(diphenylphosphino)ferrocene (144 mg, 0.26 mmol), palladium (II) acetate (29 mg, 0.13 mmol) and triethylamine (263 mg, 2.6 mmol) in ethanol (20 mL) was stirred at 70° C. for 48 hours under carbon monoxide. The reaction was cooled to room temperature and concentrated. The residue was purified by flash chromatography (silica, petroleum ether/ethyl acetate=100/95) to give ethyl 3,3-dimethyl-2,3-dihydrofuro[2,3-c]pyridine-7-carboxylate (90 mg, 0.41 mmol, 63%) as brown oil. MS (ESI) m/z 222.1 [M+H]⁺

Step 4. To a solution of ethyl 3,3-dimethyl-2,3-dihydrofuro[2,3-c]pyridine-7-carboxylate (40 mg, 0.18 mmol) in tetrahydrofuran (1 mL) were added lithium hydroxide hydrate (38 mg, 0.9 mmol) and water (1 mL). After stirring at 25° C. for 2 hours, the mixture was evaporated under reduced pressure to give lithium 3,3-dimethyl-2,3-dihydrofuro[2,3-c]pyridine-7-carboxylate (36 mg) as a white solid. The crude product was used in the next step without further purification. MS (ESI) m/z 194.2 [M+H]⁺

Step 5. A solution of 3-(5-amino-2-methylpyridin-3-yl)-N-methyl-1,6-naphthyridin-7-amine (40 mg, 0.15 mmol), lithium 3,3-dimethyl-2,3-dihydrofuro[2,3-c]pyridine-7-carboxylate (36 mg, 0.18 mmol), 1-[bis(dimethylamino)methylene]-1H-1,2,3-triazolo[4,5-b]pyridinium 3-oxid hexafluorophosphate (87 mg, 0.23 mmol), and N,N-diisopropylethylamine (77 mg, 0.6 mmol) in N,N-dimethylformamide (3 mL) was stirred at 25° C. for 3 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3,3-dimethyl-N-(6-methyl-5-(7-(methylamino)-1,6-naphthyridin-3-yl)pyridin-3-yl)-2,3-dihydrofuro[2,3-c]pyridine-7-carboxamide (12.7 mg, 0.028 mmol, 19%) as a yellow solid. ¹H NMR (400 MHz, DMSO-d₆) δ 10.66 (s, 1H), 8.99 (s, 2H), 8.87 (d, J=1.8 Hz, 1H), 8.37-8.19 (m, 3H), 7.61 (d, J=4.4 Hz, 1H), 6.96 (d, J=4.8 Hz, 1H), 6.64 (s, 1H), 4.41 (s, 2H), 2.88 (d, J=4.8 Hz, 3H), 2.49 (s, 3H), 1.35 (s, 6H). MS (EST) m/z 441.1 [M+H]⁺

### Example 100. Synthesis of 2-(2-cyanopropan-2-yl)-N-(6-(7-(methylamino)-1,6-naphthyridin-3-yl)pyridazin-4-yl)isonicotinamide (Compound 107)

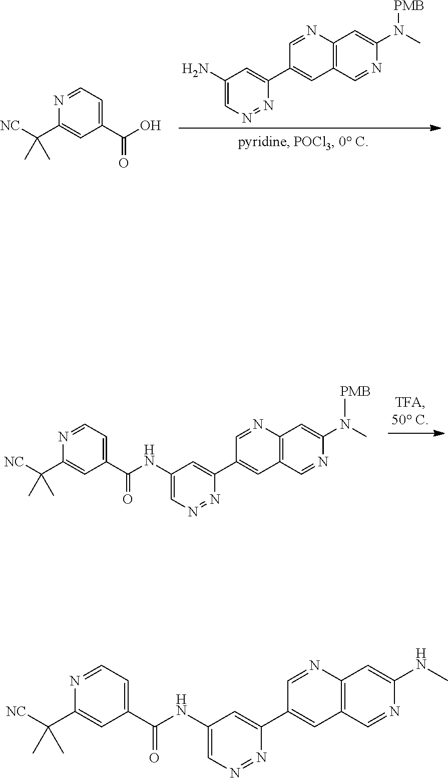

Step 1. To a solution of 3-(5-aminopyridazin-3-yl)-N-(4-methoxybenzyl)-N-methyl-1,6-naphthyridin-7-amine (90 mg, 0.24 mmol) and 2-(2-cyanopropan-2-yl)isonicotinic acid (45 mg, 0.24 mmol) in pyridine (5 mL) was added phosphoryl trichloride (3 drops) at 0° C. The mixture was stirred at 20° C. for 1 hour. The reaction solution was poured into water (20 mL) and extracted with ethyl acetate (20 mL×3). The combined organic phases were concentrated and purified by flash chromatography (silica, dichloromethane/methanol=10/1) to give 2-(2-cyanopropan-2-yl)-N-(6-(7-((4-methoxybenzyl)(methyl)amino)-1,6-naphthyridin-3-yl)pyridazin-4-yl)isonicotinamide (100 mg, 0.18 mmol, 76%) as a yellow solid. MS (ESI) m/z 545.2 [M+H]⁺

Step 2. A solution of 2-(2-cyanopropan-2-yl)-N-(6-(7-((4-methoxybenzyl)(methyl)amino)-1,6-naphthyridin-3-yl)pyridazin-4-yl)isonicotinamide (100 mg, 0.18 mmol) in trifluoroacetic acid (10 mL) was stirred at 50° C. for 1 hour. The mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%-700.% in 15 min) to give 2-(2-cyanopropan-2-yl)-N-(6-(7-(methylamino)-1,6-naphthyridin-3-yl)pyridazin-4-yl)isonicotinamide (29.2 mg, 0.068 mmol, 38%) as a yellow solid. ¹H NMR (500 MHz, DMSO-d₆) δ 11.27 (s, 1H), 9.51 (m, J=2.5 Hz, 2H), 9.13 (s, 1H), 8.90 (d, J=5.3 Hz, 2H), 8.68 (d, J=2.2 Hz, 1H), 8.09 (s, 1H), 7.95 (dd, J=5.0, 1.4 Hz, 1H), 7.14 (s, 1H), 6.66 (s, 1H), 2.89 (d, J=4.6 Hz, 3H), 1.80 (s, 6H). MS (ESI) m/z 425.1 [M+H]⁺

### Example 101. Synthesis of N-(6-(7-(methylamino)-1,6-naphthyridin-3-yl)pyridazin-4-yl)-2-(trifluoromethyl)isonicotinamide (Compound 108)

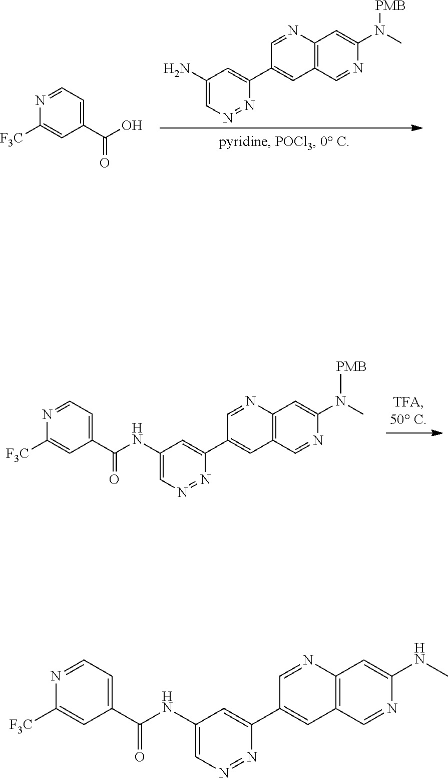

Step 1. To a solution of 3-(5-aminopyridazin-3-yl)-N-(4-methoxybenzyl)-N-methyl-1,6-naphthyridin-7-amine (90 mg, 0.24 mmol) and 2-(trifluoromethyl)isonicotinic acid (45 mg, 0.24 mmol) in pyridine (5 mL) was added phosphoryl trichloride (3 drops) at 0° C. The mixture was stirred at 20° C. for 1 hour. The reaction solution was poured into water (20 mL) and extracted with ethyl acetate (20 mL×3). The combined organic phases were concentrated and purified by flash chromatography (silica, dichloromethane/methanol=10/1) to give N-(6-(7-((4-methoxybenzyl)(methyl)amino)-1,6-naphthyridin-3-yl)pyridazin-4-yl)-2-(trifluoromethyl)isonicotinamide (110 mg, 0.20 mmol, 84%) as a yellow solid. MS (ESI) m/z 546.1 [M+H]⁺

Step 2. A solution of N-(6-(7-((4-methoxybenzyl)(methyl)amino)-1,6-naphthyridin-3-yl)pyridazin-4-yl)-2-(trifluoromethyl)isonicotinamide (110 mg, 0.20 mmol) in trifluoroacetic acid (10 mL) was stirred at 50° C. for 1 hour. The mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(6-(7-(methylamino)-1,6-naphthyridin-3-yl)pyridazin-4-yl)-2-(trifluoromethyl)isonicotinamide (21.0 mg, 0.049 mmol, 24%) as a yellow solid. ¹H NMR (500 MHz, DMSO-d₆) δ 11.36 (s, 1H), 9.50 (s, 2H), 9.24-9.05 (m, 2H), 8.88 (s, 1H), 8.67 (s, 1H), 8.44 (s, 1H), 8.26 (d, J=4.7 Hz, 1H), 7.13 (s, 1H), 6.65 (s, 1H), 2.89 (d, J=4.6 Hz, 3H). MS (ESI) m/z 426.1 [M+H]⁺

### Example 102. Synthesis of (S)-4-(1-hydroxypropyl)-N-(6-methyl-5-(7-(methylamino)-1,6-naphthyridin-3-yl)pyridin-3-yl)picolinamide and (R)-4-(1-hydroxypropyl)-N-(6-methyl-5-(7-(methylamino)-1,6-naphthyridin-3-yl)pyridin-3-yl)picolinamide (Compounds 109 and 110)

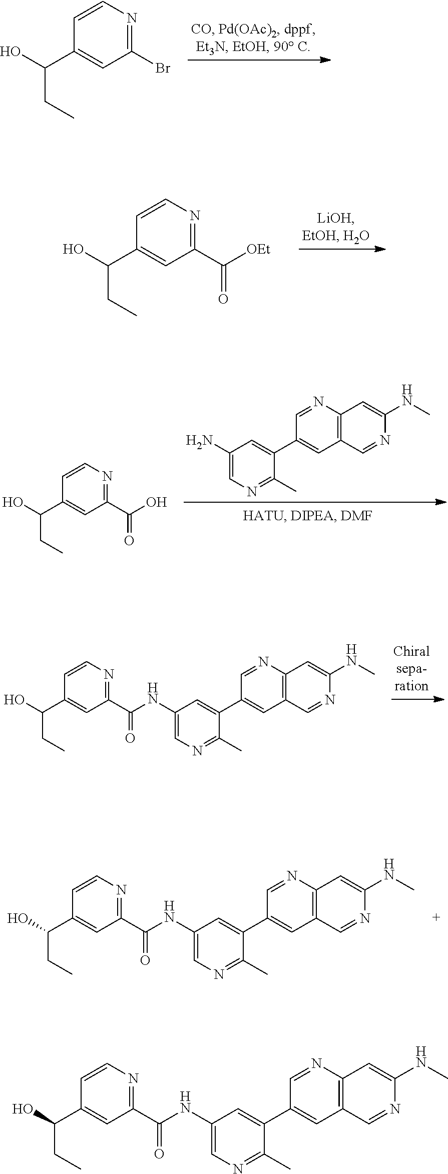

Step 1. A solution of 1-(2-bromopyridin-4-yl)propan-1-ol (100 mg, 0.47 mmol), 1,1′-bis(diphenylphosphino)ferrocene (51 mg, 0.09 mmol), palladium (II) acetate (10 mg, 0.05 mmol) and N,N-diisopropylethylamine (180 mg, 1.40 mmol) in ethanol (4 mL) was stirred at 90° C. for 16 hours under carbon monoxide atmosphere. On completion, the reaction mixture was cooled to room temperature and used in the next step without further purification. MS (ESI) m/z 210.1 [M+H]⁺

Step 2. A mixture of ethyl 4-(1-hydroxypropyl)picolinate and lithium hydroxide (59 mg, 1.40 mmol) in water (5 mL) was stirred at room temperature for 1 hour. Then the organic solvent was removed. The residue was washed with ethyl acetate (20 mL). The aqueous phase was purified by prep-HPLC to give 4-(1-hydroxypropyl)picolinic acid (30 mg, 0.17 mmol, 36% over two steps) as a white solid. MS (ESI) m/z 182.1 [M+H]⁺

Step 3. To a solution of 3-(5-amino-2-methylpyridin-3-yl)-N-methyl-1,6-naphthyridin-7-amine (44 mg, 0.17 mmol) in N,N-dimethylformamide (2 mL) was added N,N-diisopropylethylamine (64 mg, 0.50 mmol), 1-[bis(dimethylamino)methylene]-1H-1,2,3-triazolo[4,5-b]pyridinium 3-oxid hexafluorophosphate (64 mg, 0.25 mmol), and 4-(1-hydroxypropyl)picolinic acid (30 mg, 0.17 mmol). The mixture was stirred at room temperature for 2 hours. The mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(1-hydroxypropyl)-N-(6-methyl-5-(7-(methylamino)-1,6-naphthyridin-3-yl)pyridin-3-yl)picolinamide (40 mg, 0.09 mmol, 57%) as a yellow solid. MS (ESI) m/z 429.1 [M+H]⁺

Step 4. The enantiomers of 4-(1-hydroxypropyl)-N-(6-methyl-5-(7-(methylamino)-1,6-naphthyridin-3-yl)pyridin-3-yl)picolinamide (40 mg, 0.09 mmol,) were separated by chiral SFC (Instrument: SFC-150 (Waters); Column: OJ 20×250 mm, 10 μm (Daicel); Mobile phase: CO₂/MeOH (0.2% methanol ammonia)=40/60; Detection wavelength: 214 nm) to afford the first eluting enantiomer (retention time 3.85 min) which was arbitrarily assigned as (S)-4-(1-hydroxypropyl)-N-(6-methyl-5-(7-(methylamino)-1,6-naphthyridin-3-yl)pyridin-3-yl)picolinamide (12.4 mg, 0.029 mmol, 18%) as a yellow solid. ¹H NMR (500 MHz, DMSO-d6) δ 0.85 (t, J=7.0 Hz, 3H), 1.59-1.72 (m, 2H), 2.49 (s, 3H), 2.87 (d, J=5.0 Hz, 3H), 4.64 (d, J=7.0 Hz, 1H), 5.53 (d, J=4.5 Hz, 1H), 6.63 (s, 1H), 6.95 (d, J=5.0 Hz, 1H), 7.62 (d, J=3.5 Hz, 1H), 8.14 (s, 1H), 8.31 (dd, J₁=2.0 Hz, J₂=6.25 Hz, 2H), 8.68 (d, J=5.0 Hz, 1H), 8.86 (d, J=2.0 Hz, 1H), 8.99 (s, 1H), 9.04 (d, J=2.0 Hz, 1H), 10.93 (s, 1H). MS (EST) m/z 429.1 [M+H]⁺;
and the second eluting enantiomer (retention time 5.49 min) which was arbitrarily assigned as (R)-4-(1-hydroxypropyl)-N-(6-methyl-5-(7-(methylamino)-1,6-naphthyridin-3-yl)pyridin-3-yl)picolinamide (14.2 mg, 0.033 mmol, 20%) as a yellow solid. ¹H NMR (500 MHz, DMSO-d6) δ 0.85 (t, J=7.0 Hz, 3H), 1.59-1.75 (m, 2H), 2.49 (s, 3H), 2.87 (d, J=4.5 Hz, 3H), 4.64 (s, 1H), 5.53 (d, J=4.5 Hz, 1H), 6.63 (s, 1H), 6.96 (d, J=4.5 Hz, 1H), 7.62 (d, J=4.0 Hz, 1H), 8.14 (s, 1H), 8.32 (dd, J₁=2.0 Hz, J₂=7.0 Hz, 2H), 8.68 (d, J=5.0 Hz, 1H), 8.87 (d, J=2.5 Hz, 1H), 8.99 (s, 1H), 9.04 (d, J=2.0 Hz, 1H), 10.94 (s, 1H). MS (ESI) m/z 429.1[M+H]⁺

### Example 103. Synthesis of 4-hydroxy-N-(6-methyl-5-(7-(methylamino)-1,6-naphthyridin-3-yl)pyridin-3-yl)-3,4-dihydro-1H-pyrano[3,4-c]pyridine-8-carboxamide (Compound 112)

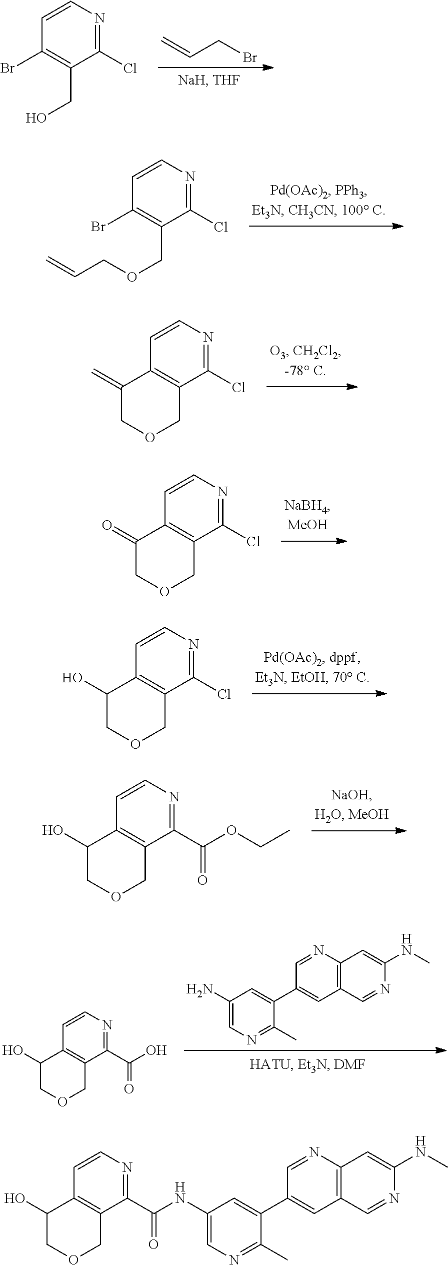

Step 1. To a solution of (4-bromo-2-chloropyridin-3-yl)methanol (500 mg, 2.26 mmol) in tetrahydrofuran (15 mL) was added sodium hydride (118 mg, 2.94 mmol, 60%) at 0° C. The mixture was stirred at 0° C. for 30 minutes. 3-Bromoprop-1-ene (326 mg, 2.71 mmol) was added and the mixture was stirred at room temperature for 18 hours. The reaction mixture was treated with water (50 mL), extracted with ethyl acetate (30 mL×2). The combined organic phases were concentrated. The residue was purified by flash chromatography (silica, petroleum ether/ethyl acetate=3/1) to give 3-((allyloxy)methyl)-4-bromo-2-chloropyridine (480 mg, 1.84 mmol, 81%) as a yellow solid. MS (ESI) m/z 262.0 [M+H]⁺

Step 2. The mixture of 3-((allyloxy)methyl)-4-bromo-2-chloropyridine (480 mg, 1.84 mmol), palladium (II) acetate (124 mg, 0.55 mmol), triphenylphosphine (145 mg, 0.55 mmol) and triethylamine (186 mg, 1.84 mmol) in acetonitrile (5 mL) in a sealed tube was stirred at 100° C. for 2 hours. The reaction mixture was cooled to room temperature and concentrated. The residue was purified by flash chromatography (silica, petroleum ether/ethyl acetate=10/1) to give 8-chloro-4-methylene-3,4-dihydro-1H-pyrano[3,4-c]pyridine (110 mg, 0.61 mmol, 33%) as a yellow solid. MS (ESI) m/z 182.1 [M+H]⁺

Step 3. Into a solution of 8-chloro-4-methylene-3,4-dihydro-1H-pyrano[3,4-c]pyridine (110 mg, 0.61 mmol) in dichloromethane (10 mL) was bubbled ozone until the solution maintained a slight blue color at −78° C. Nitrogen gas was then bubbled into the solution until it turned clear. Triphenylphosphine (318 mg, 1.22 mmol) was added to the solution and the resulting mixture was stirred at room temperature for 30 minutes. The reaction mixture was concentrated. The residue was purified by flash chromatography (silica, petroleum ether/ethyl acetate=3/1) to give 8-chloro-1H-pyrano[3,4-c]pyridin-4(3H)-one (55 mg, 0.3 mmol, 49%) as a white solid. MS (ESI) m/z 184.1 [M+H]⁺

Step 4. To a solution of 8-chloro-1H-pyrano[3,4-c]pyridin-4(3H)-one (55 mg, 0.3 mmol) in methanol (5 mL) was added sodium borohydride (23 mg, 0.6 mmol). The mixture was stirred at room temperature for 2 hours. The reaction mixture was concentrated, treated with water (20 mL) and extracted with ethyl acetate (20 mL×2). The combined organic phases were concentrated and purified by flash chromatography (silica, petroleum ether/ethyl acetate=1/1) to give 8-chloro-3,4-dihydro-1H-pyrano[3,4-c]pyridin-4-ol (45 mg, 0.24 mmol, 81%) as white solid. MS (ESI) m/z 186.1 [M+H]⁺

Step 5. The mixture of 8-chloro-3,4-dihydro-1H-pyrano[3,4-c]pyridin-4-ol (45 mg, 0.24 mmol), 1,1′-bis(diphenylphosphino)ferrocene (27 mg, 0.05 mmol), palladium (II) acetate (5 mg, 0.024 mmol) and triethylamine (74 mg, 0.73 mmol) in ethanol (5 mL) was stirred at 70° C. for 18 hours under carbon monoxide atmosphere. The reaction mixture was cooled to room temperature and concentrated. The residue was purified by flash chromatography (silica, petroleum ether/ethyl acetate=1/1) to give ethyl 4-hydroxy-3,4-dihydro-1H-pyrano[3,4-c]pyridine-8-carboxylate (30 mg, 0.13 mmol, 56%) as a yellow oil. MS (ESI) m/z 224.1 [M+H]⁺

Step 6. A mixture of ethyl 4-hydroxy-3,4-dihydro-1H-pyrano[3,4-c]pyridine-8-carboxylate (30 mg, 0.13 mmol) and sodium hydroxide (0.13 mL, 0.27 mmol, 2 M in water) in methanol (1 mL) was stirred at room temperature for 2 hours. The reaction mixture was treated with water (10 mL) and extracted with ethyl acetate (10 mL×1). The organic phase was discarded. The aqueous phase was adjusted pH=4 with hydrochloric acid (1 N) and extracted with ethyl acetate (10 mL×2). The combined organic phases were concentrated to give 4-hydroxy-3,4-dihydro-1H-pyrano[3,4-c]pyridine-8-carboxylic acid (15 mg, 0.08 mmol, 59%) as a colorless oil. The crude was used in next step without further purification. MS (ESI) m/z 196.0 [M+H]⁺

Step 7. A mixture of 4-hydroxy-3,4-dihydro-1H-pyrano[3,4-c]pyridine-8-carboxylic acid (15 mg, 0.08 mmol), 3-(5-amino-2-methylpyridin-3-yl)-N-methyl-1,6-naphthyridin-7-amine (20 mg, 0.08 mmol), 1-[bis(dimethylamino)methylene]-1H-1,2,3-triazolo[4,5-b]pyridinium 3-oxid hexafluorophosphate (35 mg, 0.09 mmol), and triethylamine (16 mg, 0.15 mmol) in N,N-dimethylformamide (2 mL) was stirred at room temperature for 2 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 4-hydroxy-N-(6-methyl-5-(7-(methylamino)-1,6-naphthyridin-3-yl)pyridin-3-yl)-3,4-dihydro-1H-pyrano[3,4-c]pyridine-8-carboxamide (3.9 mg, 0.009 mmol, 11%) as a yellow solid. ¹H NMR (500 MHz, DMSO-d₆) δ 10.93 (s, 1H), 9.06-8.92 (m, 2H), 8.86 (d, J=2.4 Hz, 1H), 8.65 (d, J=4.9 Hz, 1H), 8.29 (dd, J=19.3, 2.1 Hz, 2H), 7.76 (d, J=4.8 Hz, 1H), 6.94 (q, J=4.8 Hz, 1H), 6.64 (s, 1H), 5.88 (d, J=6.7 Hz, 1H), 5.19-5.05 (m, 2H), 4.64 (dd, J=12.2, 6.5 Hz, 1H), 3.98 (dd, J=11.2, 5.1 Hz, 1H), 3.58 (dd, J=11.2, 7.2 Hz, 1H), 2.88 (d, J=5.0 Hz, 3H), 2.48 (s, 3H). MS (ESI) m/z 443.1 [M+H]⁺

### Example 104. Synthesis of 5-(2-hydroxypropan-2-yl)-N-(6-methyl-5-(7-(methylamino)-1,6-naphthyridin-3-yl)pyridin-3-yl)nicotinamide (Compound 113)

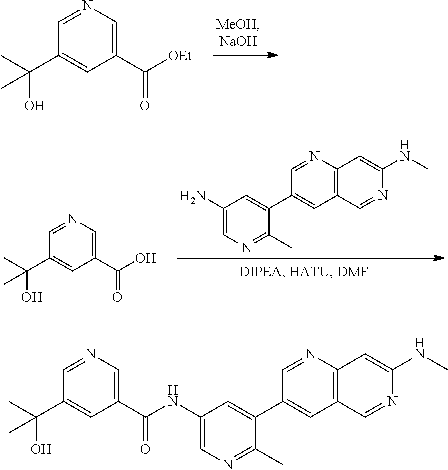 [SMILES: CCOC(=O)c1cncc(C(C)(C)O)c1 ; CC(C)(O)c1cncc(C(=O)O)c1 ; CNc1cc2ncc(-c3cc(N)cnc3C)cc2cn1 ; CNc1cc2ncc(-c3cc(NC(=O)c4cncc(C(C)(C)O)c4)cnc3C)cc2cn1]

Step 1. A solution of ethyl 5-(2-hydroxypropan-2-yl)nicotinate (80 mg, 0.38 mmol) and sodium hydroxide (18 mg, 0.76 mmol) in methanol (3 mL) and water (I mL) was stirred at room temperature for 2 hours. The solvent was evaporated under reduced pressure. The residue was diluted with water (5 mL) and adjusted to pH=3 by adding 1 N hydrochloric acid slowly. The mixture was extracted with ethyl acetate (10 mL×3). The combined organic phases were dried over sodium sulfate, filtered, and concentrated under reduced pressure to give 5-(2-hydroxypropan-2-yl)nicotinic acid (50 mg, 0.28 mmol, 74%) as a yellow solid. MS (ESI) m/z 182.2 [M+H]⁺

Step 2. A solution of 5-(2-hydroxypropan-2-yl)nicotinic acid (30 mg, 0.17 mmol), 3-(5-amino-2-methylpyridin-3-yl)-N-methyl-1,6-naphthyridin-7-amine (45 mg, 0.17 mmol), 1-[bis(dimethylamino)methylene]-1H-1,2,3-triazolo[4,5-b]pyridinium 3-oxid hexafluorophosphate (99 mg, 0.26 mmol) and N,N-diisopropylethylamine (66 mg, 0.51 mmol) in N,N-dimethylformamide (3 mL) was stirred at room temperature for 2 hours. The mixture was quenched with brine (10 mL) and extracted with ethyl acetate (10 mL×3). The combined organic phases were dried over sodium sulfate, filtered, and concentrated under reduced pressure. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5-(2-hydroxypropan-2-yl)-N-(6-methyl-5-(7-(methylamino)-1,6-naphthyridin-3-yl)pyridin-3-yl)nicotinamide (20.0 mg, 0.046 mmol, 28%) as a yellow solid. ¹H NMR (500 MHz, DMSO-d₆) δ 10.68 (s, 1H), 9.12-8.95 (m, 2H), 8.94-8.81 (m, 3H), 8.50-8.24 (m, 2H), 8.16 (d, J=2.2 Hz, 1H), 6.96 (d, J=4.9 Hz, 1H), 6.64 (s, 1H), 5.35 (d, J=44.3 Hz, 1H), 2.88 (d, J=4.9 Hz, 3H), 2.51 (s, 3H), 1.52 (s, 6H). MS (ESI) m/z 429.1 [M+H]⁺

### Example 105. Synthesis of 4-(2-(difluoromethoxy)propan-2-yl)-N-(6-methyl-5-(7-(methylamino)-1,6-naphthyridin-3-yl)pyridin-3-yl)picolinamide (Compound 114)

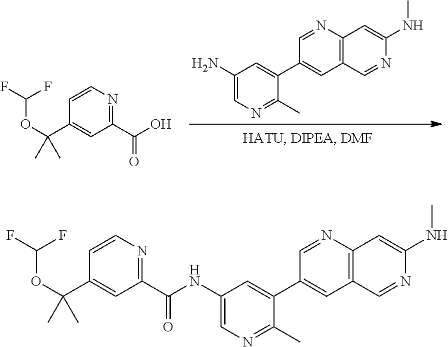 [SMILES: CC(C)(OC(F)F)c1ccnc(C(=O)O)c1 ; CNc1cc2ncc(-c3cc(N)cnc3C)cc2cn1 ; CNc1cc2ncc(-c3cc(NC(=O)c4cc(C(C)(C)OC(F)F)ccn4)cnc3C)cc2cn1]

A solution of 3-(5-amino-2-methylpyridin-3-yl)-N-methyl-1,6-naphthyridin-7-amine (32 mg, 0.12 mmol), 4-(2-(difluoromethoxy)propan-2-yl)picolinic acid (33 mg, 0.14 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (62 mg, 0.48 mmol) in N,N-dimethylformamide (3 mL) was stirred at 25° C. for 3 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%/6-70% in 15 min) to afford 4-(2-(difluoromethoxy)propan-2-yl)-N-(6-methyl-5-(7-(methylamino)-1,6-naphthyridin-3-yl)pyridin-3-yl)picolinamide (18.9 mg, 0.04 mmol, 33%) as a yellow solid. ¹H NMR (400 MHz, DMSO-d₆) δ 11.01 (s, 1H), 9.06 (d, J=2.3 Hz, 1H), 9.00 (s, 1H), 8.87 (d, J=2.3 Hz, 1H), 8.79 (d, J=5.1 Hz, 1H), 8.32 (t, J=2.6 Hz, 2H), 8.22 (d, J=1.6 Hz, 1H), 7.76 (dd, J=5.2, 1.8 Hz, 1H), 7.02-6.61 (m, 3H), 2.88 (d, J=4.9 Hz, 3H), 2.50 (s, 3H), 1.71 (s, 6H). MS (ESI) m/z 479.0 [M+H]⁺

### Example 106. Synthesis of 4-(1-amino-2,2,2-trifluoroethyl)-N-(4-methyl-3-(7-(methylamino)-1,6-naphthyridin-3-yl)phenyl)picolinamide, (R)-4-(1-amino-2,2,2-trifluoroethyl)-N-(4-methyl-3-(7-(methylamino)-1,6-naphthyridin-3-yl)phenyl)picolinamide, and (S)-4-(1-amino-2,2,2-trifluoroethyl)-N-(4-methyl-3-(7-(methylamino)-1,6-naphthyridin-3-yl)phenyl)picolinamide (Compounds 117, 115, and 116)

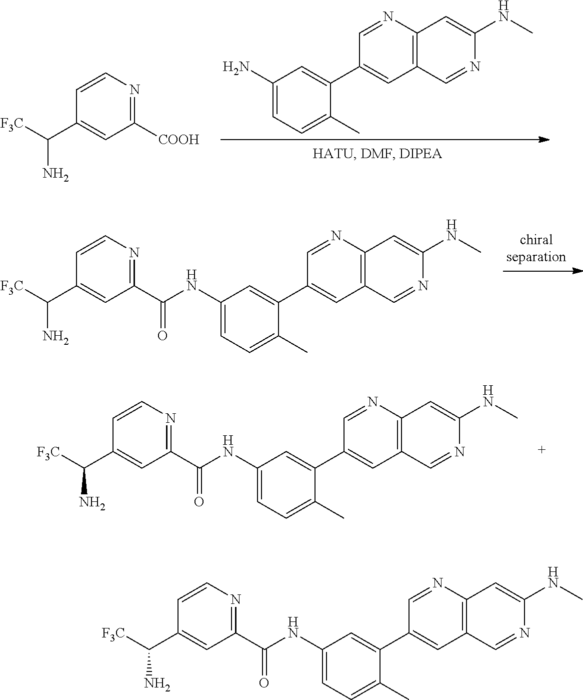 [SMILES: NC(c1ccnc(C(=O)O)c1)C(F)(F)F ; CNc1cc2ncc(-c3cc(N)ccc3C)cc2cn1 ; CNc1cc2ncc(-c3cc(NC(=O)c4cc(C(N)C(F)(F)F)ccn4)ccc3C)cc2cn1 ; CNc1cc2ncc(-c3cc(NC(=O)c4cc([C@@H](N)C(F)(F)F)ccn4)ccc3C)cc2cn1 ; CNc1cc2ncc(-c3cc(NC(=O)c4cc([C@H](N)C(F)(F)F)ccn4)ccc3C)cc2cn1]

Step 1. A solution of 4-(1-amino-2,2,2-trifluoroethyl)picolinic acid (120 mg, 0.55 mmol), 3-(5-amino-2-methylphenyl)-N-methyl-1,6-naphthyridin-7-amine (144 mg, 0.55 mmol), 1-[bis(dimethylamino)methylene]-1H-1,2,3-triazolo[4,5-b]pyridinium 3-oxid hexafluorophosphate (313 mg, 0.83 mmol), and N,N-diisopropylethylamine (0.2 mL) in N,N-dimethylformamide (5 mL) was stirred at room temperature for 12 hours. The reaction was diluted with ethyl acetate (50 mL), washed with water (20 mL) and brine (20 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(1-amino-2,2,2-trifluoroethyl)-N-(4-methyl-3-(7-(methylamino)-1,6-naphthyridin-3-yl)phenyl)picolinamide (50 mg, 0.11 mmol, 20%) as a yellow solid. ¹H NMR (400 MHz, DMSO-d₆) δ 10.69 (s, 1H), 8.97 (s, 1H), 8.83 (d, J=1.6 Hz, 1H), 8.76 (d, J=4.0 Hz, 1H), 8.31 (s, 1H), 8.24 (d, J=1.6 Hz, 1H), 7.92-7.81 (m, 2H), 7.81 (d, J=4.0 Hz, 1H), 7.36 (d, J=6.8 Hz, 1H), 6.88-6.68 (m, 1H), 6.63 (s, 1H), 4.78 (s, 1H), 2.88 (d, J=4.0 Hz, 3H), 2.77 (s, 1H), 1.99 (s, 3H). MS (ESI) m/z 467.1 [M+H]⁺

Step 2. The enantiomers of 4-(1-amino-2,2,2-trifluoroethyl)-N-(4-methyl-3-(7-(methylamino)-1,6-naphthyridin-3-yl)phenyl)picolinamide (50 mg, 0.11 mmol) were separated by chiral prep-HPLC (Instrument: Gilson-281; Column: AY 20×250 mm, 10 μm (Daicel); Mobile phase: n-hexanes (0.1% diethylamine)/EtOH (0.1% diethylamine)=10/90; Detection wavelength: 214 nm) to afford the first eluting enantiomer (retention time 6.7 min) which was arbitrarily assigned as (S)-4-(1-amino-2,2,2-trifluoroethyl)-N-(4-methyl-3-(7-(methylamino)-1,6-naphthyridin-3-yl)phenyl)picolinamide (18.7 mg, 0.040 mmol, 37%). ¹H NMR (400 MHz, DMSO-d₆) δ 10.71 (s, 1H), 8.98 (s, 1H), 8.83 (d, J=1.2 Hz, 1H), 8.77 (d, J=4.8 Hz, 1H), 8.32 (s, 1H), 8.26 (s, 1H), 7.93-7.81 (m, 2H), 7.81 (d, J=4.2 Hz, 1H), 7.36 (d, J=8.2 Hz, 1H), 6.93-6.91 (m, 1H), 6.63 (s, 1H), 4.84 (s, 1H), 2.88 (d, J=4.8 Hz, 3H), 3.18 (s, 3H). MS (EST) m/z 467.1 [M+H]⁺;
and the second eluting enantiomer (retention time 12.5 min) which was arbitrarily assigned as (R)-4-(1-amino-2,2,2-trifluoroethyl)-N-(4-methyl-3-(7-(methylamino)-1,6-naphthyridin-3-yl)phenyl)picolinamide (11.6 mg, 0.025 mmol, 23%) as a yellow solid. ¹H NMR (400 MHz, DMSO-d₆) δ 10.69 (s, 1H), 8.97 (s, 1H), 8.83 (d, J=1.2 Hz, 1H), 8.77 (d, J=4.8 Hz, 1H), 8.32 (s, 1H), 8.26 (s, 1H), 7.93-7.81 (m, 2H), 7.81 (d, J=4.8 Hz, 1H), 7.36 (d, J=8.0 Hz, 1H), 6.93-6.91 (m, 1H), 6.63 (s, 1H), 4.84 (s, 1H), 2.88 (d, J=4.8 Hz, 3H), 3.18 (s, 3H). MS (ESI) m/z 467.1 [M+H]⁺

### Example 107. Synthesis of 2-(difluoromethyl)-3-methoxy-N-(6-methyl-5-(7-(methylamino)-1,6-naphthyridin-3-yl)pyridin-3-yl)isonicotinamide (Compound 118)

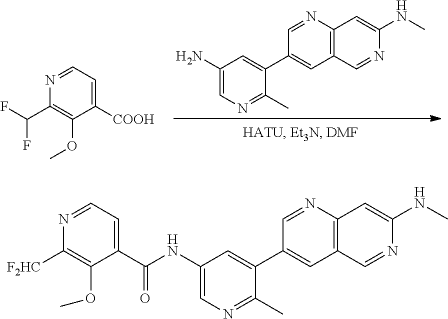 [SMILES: COc1c(C(=O)O)ccnc1C(F)F ; CNc1cc2ncc(-c3cc(N)cnc3C)cc2cn1 ; CNc1cc2ncc(-c3cc(NC(=O)c4ccnc(C(F)F)c4OC)cnc3C)cc2cn1]

A mixture of 2-(difluoromethyl)-3-methoxyisonicotinic acid (25 mg, 0.12 mmol), 3-(5-amino-2-methylpyridin-3-yl)-N-methyl-1,6-naphthyridin-7-amine (33 mg, 0.12 mmol), 1-[bis(dimethylamino)methylene]-1H-1,2,3-triazolo[4,5-b]pyridinium 3-oxid hexafluorophosphate (56 mg, 0.15 mmol), and triethylamine (37 mg, 0.37 mmol) in N,N-dimethylformamide (2 mL) was stirred at room temperature for 2 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 2-(difluoromethyl)-3-methoxy-N-(6-methyl-5-(7-(methylamino)-1,6-naphthyridin-3-yl)pyridin-3-yl)isonicotinamide (31.3 mg, 0.07 mmol, 58%) as a yellow solid. ¹H NMR (500 MHz, DMSO-d₆) δ 10.95 (s, 1H), 9.00 (s, 1H), 8.85 (d, J=2.3 Hz, 1H), 8.78 (d, J=2.4 Hz, 1H), 8.57 (d, J=4.7 Hz, 1H), 8.31 (d, J=2.3 Hz, 1H), 8.12 (d, J=2.4 Hz, 1H), 7.78 (d, J=4.7 Hz, 1H), 7.17 (t, J=53.6 Hz, 1H), 6.94 (d, J=5.0 Hz, 1H), 6.63 (s, 1H), 3.90 (s, 3H), 2.87 (d, J=4.9 Hz, 3H), 2.49 (s, 3H). MS (ESI) m/z 451.2 [M+H]⁺

### Example 108. Synthesis of 4-isopropyl-N-(6-methyl-5-(7-(methylamino)-1,6-naphthyridin-3-yl)pyridin-3-yl)picolinamide (Compound 119)

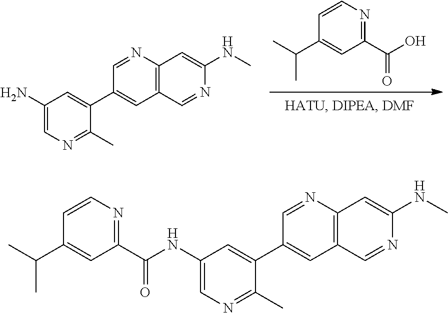

A solution of 4-isopropylpicolinic acid (56 mg, 0.34 mmol), 3-(5-amino-2-methylpyridin-3-yl)-N-methyl-1,6-naphthyridin-7-amine (53 mg, 0.2 mmol), 1-[bis(dimethylamino)methylene]-1H-1,2,3-triazolo[4,5-b]pyridinium 3-oxid hexafluorophosphate (193.8 mg, 0.51 mmol), and N,N-diisopropylethylamine (131.6 mg, 1.02 mmol) in N,N-dimethylformamide (3 mL) was stirred at room temperature for 2 hours. The reaction solution was purified by prep-HPLC (Column: Xbridge 21.2×250 mm C18, 10 μm, Mobile Phase A: water (10 mM ammonium bicarbonate), B: acetonitrile) to give 4-isopropyl-N-(6-methyl-5-(7-(methylamino)-1,6-naphthyridin-3-yl)pyridin-3-yl)picolinamide (11.4 mg, 0.028 mmol, 8%) as a yellow solid. ¹H NMR (500 MHz, DMSO-d₆) δ 10.92 (s, 1H), 9.12-8.93 (m, 2H), 8.87 (d, J=2.2 Hz, 1H), 8.65 (d, J=5.0 Hz, 1H), 8.31 (dd, J=5.8, 2.1 Hz, 2H), 8.05 (s, 1H), 7.59 (d, J=5.0 Hz, 1H), 6.94 (d, J=5.0 Hz, 1H), 6.64 (s, 1H), 3.06 (dt, J=13.9, 7.0 Hz, 1H), 2.88 (d, J=5.0 Hz, 3H), 2.49 (s, 3H), 1.26 (d, J=6.9 Hz, 6H). MS (ESI) m/z 413.1 [M+H]⁺

### Example 109. Synthesis of 3-methyl-N-(6-methyl-5-(7-(methylamino)-1,6-naphthyridin-3-yl)pyridin-3-yl)-4-(trifluoromethyl)picolinamide (Compound 120)

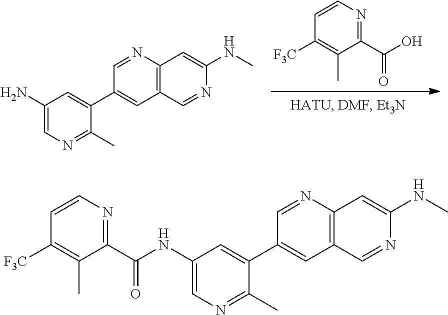

A solution of 3-methyl-4-(trifluoromethyl)picolinic acid (40 mg, 0.20 mmol), 3-(5-amino-2-methylpyridin-3-yl)-N-methyl-1,6-naphthyridin-7-amine (53 mg, 0.20 mmol), 1-[bis(dimethylamino)methylene]-1H-1,2,3-triazolo[4,5-b]pyridinium 3-oxid hexafluorophosphate (228 mg, 0.60 mmol), and triethylamine (0.2 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3-methyl-N-(6-methyl-5-(7-(methylamino)-1,6-naphthyridin-3-yl)pyridin-3-yl)-4-(trifluoromethyl)picolinamide (14.1 mg, 0.03 mmol, 16%) as a yellow solid. ¹H NMR (500 MHz, DMSO-d₆) δ 10.94 (s, 1H), 9.00 (s, 1H), 8.88-8.85 (m, 2H), 8.79 (d, J=5.0 Hz, 1H), 8.32 (d, J=2.1 Hz, 1H), 8.20 (d, J=2.3 Hz, 1H), 7.91 (d, J=5.0 Hz, 1H), 6.96-6.91 (m, 1H), 6.64 (s, 1H), 2.88 (d, J=4.9 Hz, 3H), 2.59 (s, 3H), 2.49 (s, 3H). MS (ESI) m/z 453.0 [M+H]⁺

### Example 110. Synthesis of N-methyl-3-(2-methyl-5-((8-(trifluoromethyl)imidazo[1,5-a]pyridin-3-yl)amino)pyridin-3-yl)-1,6-naphthyridin-7-amine (Compound 121)

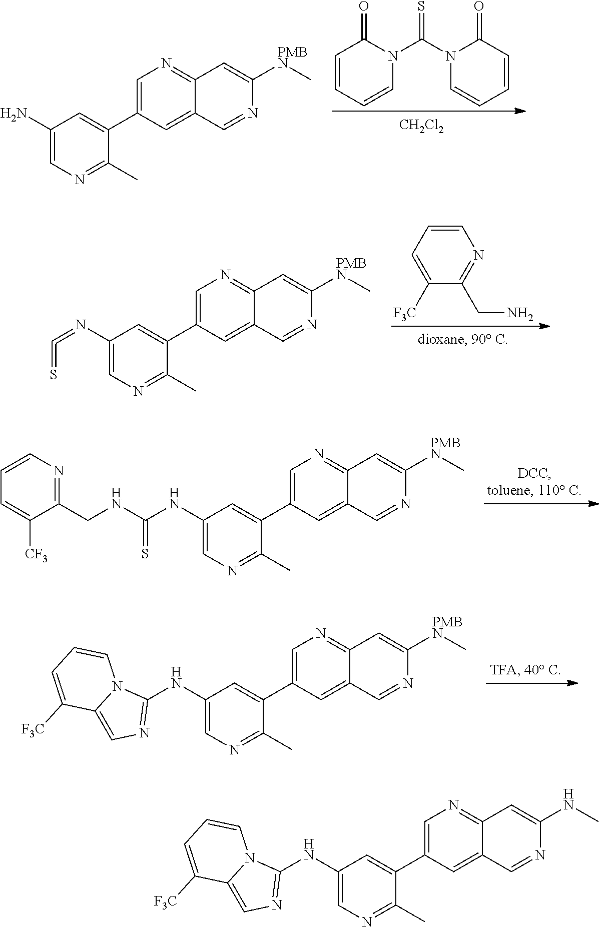

Step 1. A mixture of 3-(5-amino-2-methylpyridin-3-yl)-N-(4-methoxybenzyl)-N-methyl-1,6-naphthyridin-7-amine (500 mg, 1.3 mmol) and 1,1′-thiocarbonyldipyridin-2(1H)-one (600 mg, 2.6 mmol) in dichloromethane (20 mL) was stirred at room temperature overnight. After completion, the reaction mixture was concentrated. The residue was purified by flash chromatography (silica, dichloromethane/methanol=4/1) to afford 3-(5-isothiocyanato-2-methylpyridin-3-yl)-N-(4-methoxybenzyl)-N-methyl-1,6-naphthyridin-7-amine (490 mg, 1.15 mmol, 88%) as a yellow solid. MS (ESI) m/z 428.2 [M+H]⁺

Step 2. A mixture of 3-(5-isothiocyanato-2-methylpyridin-3-yl)-N-(4-methoxybenzyl)-N-methyl-1,6-naphthyridin-7-amine (60 mg, 0.14 mmol) and (3-(trifluoromethyl)pyridin-2-yl)methanamine (74 mg, 0.28 mmol) in 1,4-dioxane (3 mL) was stirred at 90° C. for 2 hours. After cooling to room temperature, the reaction solution was concentrated. The residue was purified by flash chromatography (silica, dichloromethane/methanol=4/1) to afford 1-(5-(7-((4-methoxybenzyl)(methyl)amino)-1,6-naphthyridin-3-yl)-6-methylpyridin-3-yl)-3-((3-(trifluoromethyl)pyridin-2-yl)methyl)thiourea (60 mg, 0.01 mmol, 71%) as a yellow solid. MS (ESI) m/z 603.9 [M+H]⁺

Step 3. A mixture of 1-(5-(7-((4-methoxybenzyl)(methyl)amino)-1,6-naphthyridin-3-yl)-6-methylpyridin-3-yl)-3-((3-(trifluoromethyl)pyridin-2-yl)methyl)thiourea (60 mg, 0.01 mmol) and N,N-dicyclohexylcarbodiimide (31 mg, 0.21 mmol) in toluene (3 mL) was heated at reflux for 2 hours. After cooling to room temperature, the reaction solution was concentrated. The residue was purified by flash chromatography (silica, petroleum ether/ethyl acetate=4/1) to give N-(4-methoxybenzyl)-N-methyl-3-(2-methyl-5-((8-(trifluoromethyl)imidazo[1,5-a]pyridin-3-yl)amino)pyridin-3-yl)-1,6-naphthyridin-7-amine (50 mg, 0.088 mmol, 88%) as a yellow solid. MS (ESI) m/z 570.1 [M+H]⁺

Step 4. A solution of N-(4-methoxybenzyl)-N-methyl-3-(2-methyl-5-((8-(trifluoromethyl)imidazo[1,5-a]pyridin-3-yl)amino)pyridin-3-yl)-1,6-naphthyridin-7-amine (50 mg, 0.088 mmol) in trifluoroacetic acid (5 mL) was stirred at 40° C. for 1 hour. The reaction mixture was concentrated and the residue was purified by prep-HPLC (Column: Waters Xbridge 150×25 mm, 5 μm, Mobile phase: A: water (0.05% ammonia hydroxide v/v), B: acetonitrile; B %: 30%-57% in 10 min) to give N-methyl-3-(2-methyl-5-((8-(trifluoromethyl)imidazo[1,5-a]pyridin-3-yl)amino)pyridin-3-yl)-1,6-naphthyridin-7-amine (28.1 mg, 0.062 mmol, 71%) as a yellow solid. ¹H NMR (400 MHz, DMSO-d₆) δ 9.33 (s, 1H), 8.98 (s, 1H), 8.83 (d, J=2.1 Hz, 1H), 8.62 (d, J=2.2 Hz, 1H), 8.34 (d, J=7.4 Hz, 1H), 8.28 (s, 1H), 7.99 (d, J=2.1 Hz, 1H), 7.26 (s, 1H), 7.13 (d, J=6.7 Hz, 1H), 6.93 (d, J=5.0 Hz, 1H), 6.73 (t, J=7.0 Hz, 1H), 6.63 (s, 1H), 2.87 (d, J=4.8 Hz, 3H), 2.43 (s, 3H). MS (ESI) m/z 450.1 [M+H]⁺

### Example 111. Synthesis of N-(6-methyl-5-(7-(methylamino)-1,6-naphthyridin-3-yl)pyridin-3-yl)-4-(2,2,2-trifluoro-1-methoxyethyl)picolinamide (Compound 122)

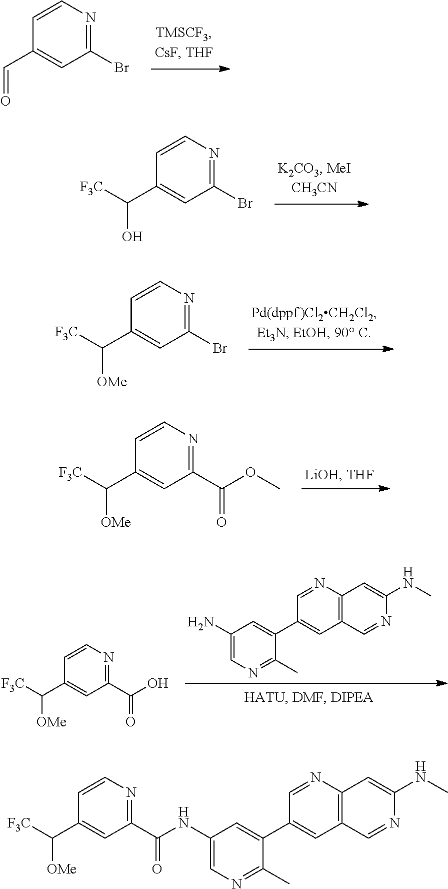

Step 1. To a solution of 2-bromoisonicotinaldehyde (2000 mg, 10.75 mmol) and cesium fluoride (1634 mg, 10.75 mmol) in tetrahydrofuran (10 mL) was added (trifluoromethyl)trimethylsilane (1527 mg, 10.75 mmol) at room temperature. After stirring at room temperature for 4 hours, the reaction mixture was quenched with water (20 mL) and extracted with ethyl acetate (30 mL×2). The combined organic phases were concentrated and purified by flash chromatography (silica, petroleum ether/ethyl acetate=3/1) to give 1-(2-bromopyridin-4-yl)-2,2,2-trifluoroethan-1-ol (2000 mg, 7.81 mmol, 73%) as a colorless oil. MS (ESI) m/z 256.0 [M+H]⁺

Step 2. A solution of 1-(2-bromopyridin-4-yl)-2,2,2-trifluoroethan-1-ol (2000 mg, 7.81 mmol), iodomethane (3327 mg, 23.43 mmol) and potassium carbonate (3233 mg, 23.43 mmol) in acetonitrile (10 mL) was stirred at 25° C. for 16 hours. The reaction was quenched with ice water (30 mL) and extracted with ethyl acetate (30 mL×2). The combined organic phases were concentrated and purified by flash chromatography (silica, petroleum ether/ethyl acetate=1/1) to give 2-bromo-4-(2,2,2-trifluoro-1-methoxyethyl)pyridine (1500 mg, 5.58 mmol, 71%) as a yellow oil. MS (ESI) m/z 270.0 [M+H]⁺

Step 3. A solution of 2-bromo-4-(2,2,2-trifluoro-1-methoxyethyl)pyridine (300 mg, 1.12 mmol), 1,1′-bis(diphenylphosphino)ferrocene-palladium(I)dichloride dichloromethane complex (90 mg, 0.11 mmol) and triethylamine (339 mg, 3.36 mmol) in ethanol (15 mL) was stirred at 90° C. for 16 hours under carbon monoxide atmosphere. The reaction was cooled to room temperature and concentrated. The residue was dissolved in water (15 mL) and extracted with ethyl acetate (30 mL×2). The combined organic phases were concentrated and purified by flash chromatography (silica, petroleum ether/ethyl acetate=1/1) to give ethyl 4-(2,2,2-trifluoro-1-methoxyethyl)picolinate (200 mg, 0.76 mmol, 69%) as a brown oil. MS (ESI) m/z 264.1 [M+H]⁺

Step 4. A mixture of ethyl 4-(2,2,2-trifluoro-1-methoxyethyl)picolinate (200 mg, 0.76 mmol) and lithium hydroxide (64 mg, 1.52 mmol) in tetrahydrofuran (5 mL) was stirred at room temperature for 1 hour. The reaction mixture was extracted with ethyl acetate (30 mL×2). The water layer was adjusted to pH=4 with hydrochloric acid (6 N) and extracted with ethyl acetate (30 mL×2). The combined organic phases were concentrated to give 4-(2,2,2-trifluoro-1-methoxyethyl)picolinic acid (150 mg, 0.77 mmol, 64%) as a brown oil. MS (ESI) m/z 236.1 [M+H]⁺

Step 5. A mixture of 4-(2,2,2-trifluoro-1-methoxyethyl)picolinic acid (55 mg, 0.25 mmol), 3-(5-amino-2-methylphenyl)-N-methyl-1,6-naphthyridin-7-amine (66 mg, 0.25 mmol), 1-[bis(dimethylamino)methylene]-1H-1,2,3-triazolo[4,5-b]pyridinium 3-oxid hexafluorophosphate (104 mg, 0.27 mmol), and N,N-diisopropylethylamine (64 mg, 0.5 mmol) in N,N-dimethylformamide (3 mL) was stirred at room temperature for 1 hour. The reaction mixture was treated with water (50 mL). The precipitate was collected by filtration, washed with water (5 mL) and dried to give N-(6-methyl-5-(7-(methylamino)-1,6-naphthyridin-3-yl)pyridin-3-yl)-4-(2,2,2-trifluoro-1-methoxyethyl)picolinamide (46 mg, 0.10 mmol, 50%) as a yellow solid. ¹H NMR (400 MHz, DMSO-d₆) δ 11.04 (s, 1H), 9.07-8.97 (m, 2H), 8.87 (t, J=3.5 Hz, 2H), 8.32 (dd, J=6.2, 2.2 Hz, 2H), 8.25 (s, 1H), 7.78 (d, J=4.9 Hz, 1H), 6.96 (d, J=5.0 Hz, 1H), 6.64 (s, 1H), 5.46 (q, J=6.9 Hz, 1H), 3.45 (s, 3H), 2.88 (d, J=4.9 Hz, 3H), 2.50 (s, J=3.4, 1.7 Hz, 3H). MS (ESI) m/z 483.0 [M+H]⁺

### Example 112. Synthesis of N-(6-methyl-5-(7-(methylamino)-1,6-naphthyridin-3-yl)pyridin-3-yl)-2-(2,2,2-trifluoro-1-methoxyethyl)isonicotinamide (Compound 123)

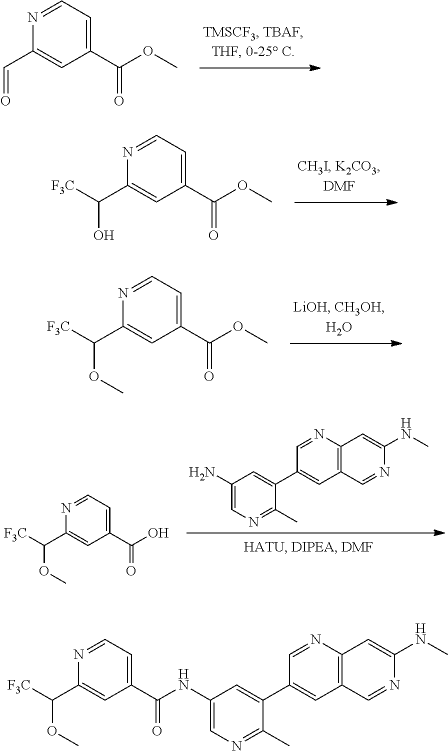 [SMILES: COC(=O)c1ccnc(C=O)c1 ; COC(=O)c1ccnc(C(O)C(F)(F)F)c1 ; COC(=O)c1ccnc(C(OC)C(F)(F)F)c1 ; COC(c1cc(C(=O)O)ccn1)C(F)(F)F ; CNc1cc2ncc(-c3cc(N)cnc3C)cc2cn1 ; CNc1cc2ncc(-c3cc(NC(=O)c4ccnc(C(OC)C(F)(F)F)c4)cnc3C)cc2cn1]

Step 1. To a stirred solution of methyl 2-formylisonicotinate (330 mg, 2 mmol) and (trifluoromethyl)trimethylsilane (341 mg, 2.4 mmol) in tetrahydrofuran (5 mL) at 0° C. was add 1 M tetrabutylammonium fluoride in tetrahydrofuran (0.1 mL, 0.1 mmol). The reaction solution was stirred at 0° C. for 30 minutes and then at room temperature for 1 hour. The reaction solution was quenched with 1 N aqueous hydrogen chloride (2 mL) and stirred 2 hours at room temperature. 1 N aqueous sodium hydroxide was added to the solution until pH=8 and extract with ethyl acetate (50 mL×3). The combined organic layers were dried over anhydrous sodium sulfate, filtered, and concentrate in vacuo. The residue was purified by flash chromatography (silica, hexanes/ethyl acetate=5/1) to give methyl 2-(2,2,2-trifluoro-1-hydroxyethyl)isonicotinate (260 mg, 1.1 mmol, 55%) as a colorless oil. MS (ESI) m/z 236.2 [M+H]⁺

Step 2. To a stirred solution of methyl 2-(2,2,2-trifluoro-1-hydroxyethyl)isonicotinate (260 mg, 1.1 mmol) and potassium carbonate (304 mg, 2.2 mmol) in tetrahydrofuran (6 mL) at 0° C. was added iodomethane (187 mg, 1.33 mmol). After stirring at room temperature for 16 hours, the solvent was removed. The residue was purified by flash chromatography (silica, hexanes/ethyl acetate=6/1) to give methyl 2-(2,2,2-trifluoro-1-methoxyethyl)isonicotinate (170 mg, 0.68 mmol, 62%) as a colorless oil. MS (EST) m/z 250.1 [M+H]⁺

Step 3. A mixture of methyl 2-(2,2,2-trifluoro-1-methoxyethyl)isonicotinate (60 mg, 0.24 mmol) and lithium hydroxide (12 mg, 0.48 mmol) in methanol (4 mL) and water (2 mL) was stirred at room temperature for 1 hour. The organic solvent was removed. The residue was diluted with water (2 mL), acidified with 1 N hydrogen chloride till no more precipitate formed and filtered. The solid was washed with water (10 mL) and dried to give 2-(2,2,2-trifluoro-1-methoxyethyl)isonicotinic acid (50 mg, 0.21 mmol, 88%) as a white solid. MS (ESI) m/z 236.1 [M+H]⁺

Step 4. A mixture of 2-(2,2,2-trifluoro-1-methoxyethyl)isonicotinic acid (50 mg, 0.21 mmol), 3-(5-amino-2-methylpyridin-3-yl)-N-methyl-1,6-naphthyridin-7-amine (56 mg, 0.21 mmol), 1-[bis(dimethylamino)methylene]-1H-1,2,3-triazolo[4,5-b]pyridinium 3-oxid hexafluorophosphate (97 mg, 0.25 mmol), and N,N-diisopropylethylamine (55 mg, 0.42 mmol) in N,N-dimethylformamide (5 mL) was stirred at room temperature for 4 hours. The mixture was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-2-(2,2,2-trifluoro-1-methoxyethyl)isonicotinamide (50 mg, 0.1 mmol, 49%) as a yellow solid. ¹H NMR (500 MHz, DMSO-d₆) δ 10.87 (s, 1H), 9.02 (s, 1H), 8.91-8.89 (m, 3H), 8.37 (s, 1H), 8.18 (s, 1H), 8.09 (s, 1H), 8.02 (dd, J=5.0 Hz, 1.5 Hz, 1H), 7.03 (s, 1H), 6.64 (s, 1H), 5.22 (q, J=7.0 Hz, 1H), 3.48 (s, 3H), 2.89 (d, J=3.0 Hz, 3H), 2.55 (s, 3H). MS (ESI) m/z 483.1 [M+H]⁺

### Example 113. Synthesis of N-(6-methyl-5-(7-(methylamino)-1,6-naphthyridin-3-yl)pyridin-3-yl)-4-(2,2,2-trifluoro-1-hydroxyethyl)picolinamide (Compound 124)

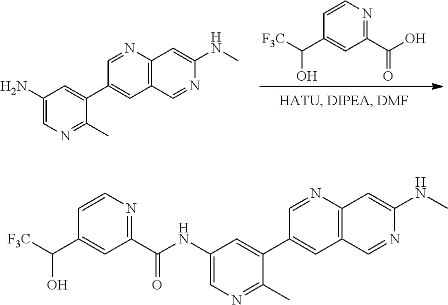 [SMILES: CNc1cc2ncc(-c3cc(N)cnc3C)cc2cn1 ; O=C(O)c1cc(C(O)C(F)(F)F)ccn1 ; CNc1cc2ncc(-c3cc(NC(=O)c4cc(C(O)C(F)(F)F)ccn4)cnc3C)cc2cn1]

A solution of 4-(2,2,2-trifluoro-1-hydroxyethyl)picolinic acid (100 mg, 0.45 mmol), 3-(5-amino-2-methylpyridin-3-yl)-N-methyl-1,6-naphthyridin-7-amine (119 mg, 0.45 mmol), 1-[bis(dimethylamino)methylene]-1H-1,2,3-triazolo[4,5-b]pyridinium 3-oxid hexafluorophosphate (228 mg, 0.60 mmol), and N,N-diisopropylethylamine (174 mg, 1.35 mmol) in N,N-dimethylformamide (5 mL) was stirred at room temperature for 2 hours. The mixture was quenched with brine (20 mL) and extracted with ethyl acetate (10 mL×3). The combined organic phases were dried over sodium sulfate, filtered, and concentrated under reduced pressure. The residue was purified by prep-HPLC (Column: Welch Xtimate 21.2×250 mm C18, 10 μm, Mobile Phase: A: water (10 mM ammonium bicarbonate), B: acetonitrile; B %: 30%-70/a in 15 min) to give N-(6-methyl-5-(7-(methylamino)-1,6-naphthyridin-3-yl)pyridin-3-yl)-4-(2,2,2-trifluoro-1-hydroxyethyl)picolinamide (2.2 mg, 0.005 mmol, 1%) as a yellow solid. ¹H NMR (400 MHz, DMSO-d₆) δ 11.02 (s, 1H), 9.04 (d, J=2.4 Hz, 1H), 8.99 (s, 1H), 8.87 (d, J=2.3 Hz, 1H), 8.83 (d, J=5.1 Hz, 1H), 8.32 (dd, J=5.4, 2.2 Hz, 3H), 7.82 (d, J=4.6 Hz, 1H), 6.97 (d, J=5.0 Hz, 1H), 6.64 (s, 1H), 5.52 (t, J=7.4 Hz, 1H), 2.88 (d, J=5.0 Hz, 3H), 2.51 (s, 3H). MS (ESI) m/z 468.9 [M+H]⁺

### Example 114. Synthesis of (S)-2-((1,1-difluoropropan-2-yl)amino)-N-(6-methyl-5-(7-(methylamino)-1,6-naphthyridin-3-yl)pyridin-3-yl)nicotinamide and (R)-2-((1,1-difluoropropan-2-yl)amino)-N-(6-methyl-5-(7-(methylamino)-1,6-naphthyridin-3-yl)pyridin-3-yl)nicotinamide (Compounds 125 and 126)

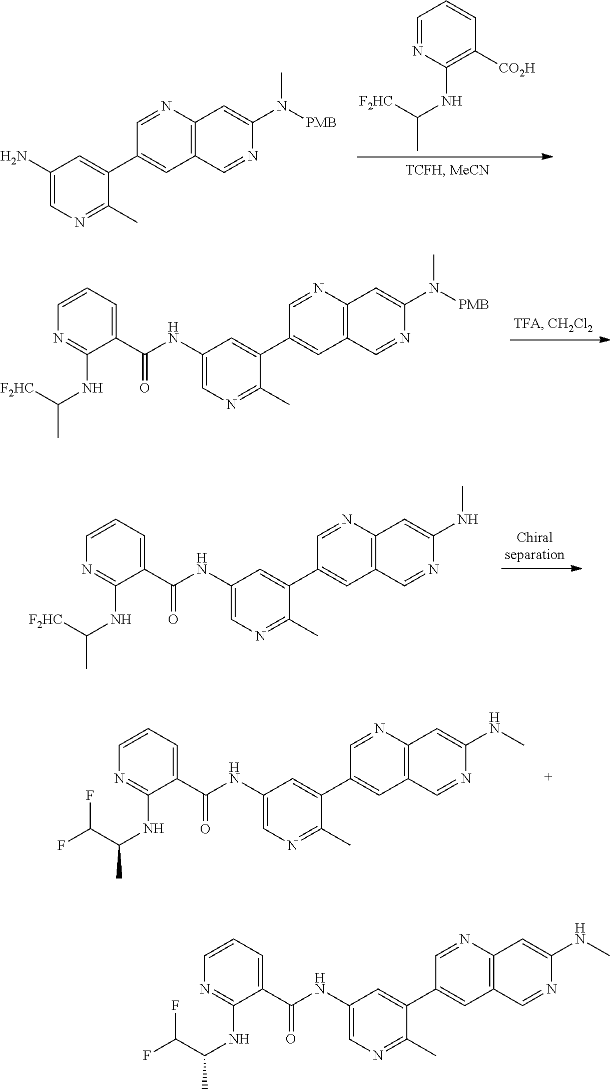

Step 1. To a solution of 3-(5-amino-2-methyl-3-pyridyl)-N-[(4-methoxyphenyl)methyl]-N-methyl-1,6-naphthyridin-7-amine (0.18 g, 0.47 mmol) in acetonitrile (2 mL) was added 2-[(2,2-difluoro-1-methyl-ethyl)amino]pyridine-3-carboxylic acid (0.1 g, 0.47 mmol), Chloro-N,N,N′,N′-tetramethylformamidinium hexafluorophosphate (TCFH, 0.26 g, 0.93 mmol) and 1-methylimidazole (0.15 g, 1.9 mmol). The mixture was stirred at 25° C. for 2 hours. On completion, the reaction mixture was concentrated under vacuum. The residue was purified by reversed-phase HPLC (0.1% formic acid condition) to give 2-((1,1-difluoropropan-2-yl)amino)-N-(5-(7-((4-methoxybenzyl)(methyl)amino)-1,6-naphthyridin-3-yl)-6-methylpyridin-3-yl)nicotinamide (0.26 g, 95%) as a yellow solid. MS (EST) m/z 584.3 [M+H]⁺

Step 2. To a solution of 2-[(2,2-difluoro-1-methyl-ethyl)amino]-N-[5-[7-[(4-methoxyphenyl)methyl-methyl-amino]-1,6-naphthyridin-3-yl]-6-methyl-3-pyridyl]pyridine-3-carboxamide (0.26 g, 0.45 mmol) in dichloromethane (3 mL) was added trifluoroacetic acid (1.5 g, 14 mmol). The mixture was stirred at 25° C. for 1 hour. The mixture was concentrated in vacuo and the resulting residue was purified by reversed-phase HPLC (0.1% formic acid condition) to give 2-((1,1-difluoropropan-2-yl)amino)-N-(6-methyl-5-(7-(methylamino)-1,6-naphthyridin-3-yl)pyridin-3-yl)nicotinamide (0.18 g, 87%) as a yellow solid. MS (ESI) m/z 464.2 [M+H]⁺

Step 3. The enantiomers of 2-((1,1-difluoropropan-2-yl)amino)-N-(6-methyl-5-(7-(methylamino)-1,6-naphthyridin-3-yl)pyridin-3-yl)nicotinamide (0.18 g) were separated by chiral SFC (Column: ChiralPak IG 30×250 mm, 10 μm (Daicel); Mobile phase: CO₂/MeOH-acetonitrile=60/40; Detection wavelength: 214 nm) to afford the first eluting enantiomer (retention time 1.3 min) which was arbitrarily assigned as (S)-2-((1,1-difluoropropan-2-yl)amino)-N-(6-methyl-5-(7-(methylamino)-1,6-naphthyridin-3-yl)pyridin-3-yl)nicotinamide (54 mg, 30%) as a yellow solid. ¹H NMR (400 MHz, DMSO-d₆) δ 10.50 (s, 1H), 8.99 (s, 1H), 8.85 (d, J=2.4 Hz, 1H), 8.78 (d, J=2.4 Hz, 1H), 8.31 (d, J=1.6 Hz, 1H), 8.27 (dd, J=1.6, 4.8 Hz, 1H), 8.23-8.14 (m, 2H), 8.11 (d, J=2.4 Hz, 1H), 6.94 (q, J=4.8 Hz, 1H), 6.78 (dd, J=4.8, 7.6 Hz, 1H), 6.63 (s, 1H), 6.34-5.90 (m, 1H), 4.79-4.62 (m, 1H), 2.87 (d, J=5.2 Hz, 3H), 2.48 (br s, 3H), 1.22 (d, J=6.8 Hz, 3H). MS (ESI) m/z 464.0 [M+H]⁺;
and the second eluting enantiomer (retention time 1.6 min) which was arbitrarily assigned as (R)-2-((1,1-difluoropropan-2-yl)amino)-N-(6-methyl-5-(7-(methylamino)-1,6-naphthyridin-3-yl)pyridin-3-yl)nicotinamide (65 mg, 36%) as a yellow solid. ¹H NMR (400 MHz, DMSO-d₆) δ 10.50 (br s, 1H), 8.99 (s, 1H), 8.85 (d, J=2.4 Hz, 1H), 8.78 (d, J=2.4 Hz, 1H), 8.31 (d, J=1.6 Hz, 1H), 8.29-8.24 (m, 1H), 8.21 (dd, J=1.6, 7.6 Hz, 1H), 8.17 (d, J=8.4 Hz, 1H), 8.11 (d, J=2.4 Hz, 1H), 6.94 (q, J=4.8 Hz, 1H), 6.78 (dd, J=4.8, 7.6 Hz, 1H), 6.63 (s, 1H), 6.32-5.96 (m, 1H), 4.78-4.62 (m, 1H), 2.87 (d, J=5.2 Hz, 3H), 2.48 (br s, 3H), 1.22 (d, J=6.8 Hz, 3H). MS (ESI) m/z 464.0 [M+H]⁺

### Example 115. Synthesis of 4-(1-(difluoromethyl)cyclopropyl)-N-(4-methyl-3-(7-(methylamino)-1,6-naphthyridin-3-yl)phenyl)picolinamide (Compound 127)

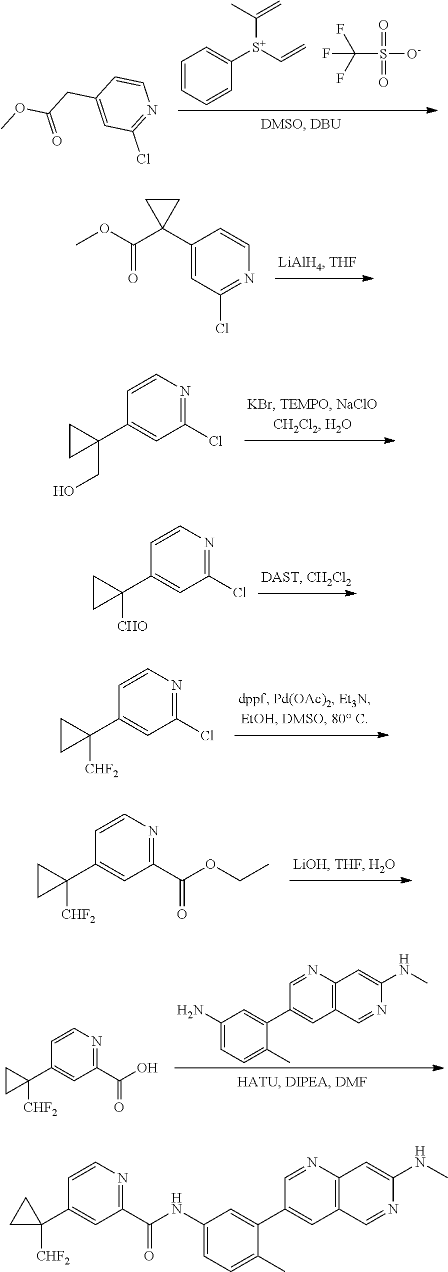

Step 1. A solution of methyl 2-(2-chloropyridin-4-yl)acetate (4 g, 21.6 mmol), diphenyl(vinyl)sulfonium trifluoromethanesulfonate (7.82 g, 21.6 mmol) and 1,8-diazabicyclo[5.4.0]undec-7-ene (6.56 g, 43.2 mmol) in dimethyl sulfoxide (40 mL) was stirred at room temperature overnight. The mixture was poured into water (50 mL) and extracted with ethyl acetate (50 mL×3). The combined organic phases were washed with brine, dried over sodium sulfate, filtered, and concentrated. The residue was purified by flash chromatography (silica, petroleum ether/ethyl acetate=1/1) to give methyl 1-(2-chloropyridin-4-yl)cyclopropane-1-carboxylate (4 g, 18.9 mmol, 88%) as a colorless oil. MS (ESI) m/z 212.2 [M+H]⁺

Step 2. To a solution of methyl 1-(2-chloropyridin-4-yl)cyclopropane-1-carboxylate (5 g, 23.6 mmol) in tetrahydrofuran (50 mL) was added lithium aluminum hydride solution (1.0 M solution in tetrahydrofuran, 35.4 mL) dropwise at room temperature. After stirring at room temperature for 2 hours, the reaction solution was poured into ice water (50 mL) and adjusted to pH 5.0 with dilute hydrochloric acid. The mixture was extracted with ethyl acetate (50 mL×3). The combined organic phases were washed with brine (50 mL), dried over sodium sulfate, filtered, concentrated and purified by flash chromatography (silica, petroleum ether/ethyl acetate=1/2) to give (1-(2-chloropyridin-4-yl)cyclopropyl)methanol (2.1 g, 11.47 mmol, 48%) as a colorless oil. MS (ESI) m/z 184.1 [M+H]⁺

Step 3. A solution of (1-(2-chloropyridin-4-yl)cyclopropyl)methanol (900 mg, 4.91 mmol), potassium bromide (117 mg, 0.98 mmol), sodium bicarbonate (83 mg, 0.98 mmol), 2,2,6,6-tetramethyl-1-piperidinyloxy (77 mg, 0.5 mmol), and sodium hypochlorite (545 mg, 1.48 mmol) in dichloromethane (10 mL) and water (2 mL) was stirred at room temperature for 2 hours. The reaction mixture was concentrated. The residue was purified by flash chromatography (silica, petroleum ether/ethyl acetate=1/1) to give 1-(2-chloropyridin-4-yl)cyclopropane-1-carbaldehyde (500 mg, 2.76 mmol, 56%) as a colorless oil. MS (ESI) m/z 182.1 [M+H]⁺

Step 4. A solution of 1-(2-chloropyridin-4-yl)cyclopropane-1-carbaldehyde (500 mg, 2.76 mmol) and diethylaminosulfur trifluoride (888 mg, 5.52 mmol) in dichloromethane (10 mL) was stirred at room temperature overnight. The mixture was treated with saturated aqueous sodium bicarbonate (30 mL) and extracted with dichloromethane (30 mL×3). The combined organic phases were washed with brine (30 mL), dried over sodium sulfate, filtered, concentrated and purified by flash chromatography (10% ethyl acetate in petroleum ether) to give 2-chloro-4-(1-(difluoromethyl)cyclopropyl)pyridine (5.7 g, 14 mmol, 81%) as a yellow oil. MS (ESI) m/z 204.1 [M+H]⁺

Step 5. A solution of 2-chloro-4-(1-(difluoromethyl)cyclopropyl)pyridine (300 mg, 1.47 mmol), 1,1′-bis(diphenylphosphino)ferrocene (163 mg, 0.3 mmol), palladium (II) acetate (34 mg, 0.15 mmol) and triethylamine (445 mg, 4.41 mmol) in ethanol (5 mL) and dimethyl sulfoxide (5 mL) was stirred at 80° C. overnight under carbon monoxide atmosphere. After cooling to room temperature, the mixture was poured into water (30 mL) and extracted with ethyl acetate (30 mL×3). The combined organic phases were washed with brine (30 mL), dried over sodium sulfate, filtered, concentrated and purified by flash chromatography (20% ethyl acetate in petroleum ether) to give ethyl 4-(1-(difluoromethyl)cyclopropyl)picolinate (150 mg, 0.62 mmol, 42%) as a yellow oil. MS (EST) m/z 242.1[M+H]⁺

Step 6. A solution of ethyl 4-(1-(difluoromethyl)cyclopropyl)picolinate (150 mg, 0.62 mmol) and lithium hydroxide (74.4 mg, 0.95 mmol) in tetrahydrofuran (3 mL) and water (3 mL) was stirred at room temperature overnight. The reaction mixture was quenched with water (10 mL) and extracted with ethyl acetate (10 mL). The aqueous phase was adjusted to pH=4.0 with dilute hydrochloric acid and extracted with ethyl acetate (10 mL×3). The combined organic phases were dried over anhydrous sodium sulfate, filtered, and concentrated to give 4-(1-(difluoromethyl)cyclopropyl)picolinic acid (130 mg, 0.61 mmol, 98%) as a white solid, which was used without further purification. MS (ESI) m/z 214.3 [M+H]⁺

Step 7. A mixture of 4-(1-(difluoromethyl)cyclopropyl)picolinic acid (30 mg, 0.14 mmol), 3-(5-amino-2-methylphenyl)-N-methyl-1,6-naphthyridin-7-amine (37 mg, 0.14 mmol), 1-[bis(dimethylamino)methylene]-1H-1,2,3-triazolo[4,5-b]pyridinium 3-oxid hexafluorophosphate (106 mg, 0.28 mmol), and N,N-diisopropylethylamine (54 mg, 0.42 mmol) in N,N-dimethylformamide (1 mL) was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 4-(1-(difluoromethyl)cyclopropyl)-N-(4-methyl-3-(7-(methylamino)-1,6-naphthyridin-3-yl)phenyl)picolinamide (5.4 mg, 0.01 mmol, 8%) as a yellow solid. ¹H NMR (400 MHz, DMSO-d₆) δ 10.70 (s, 1H), 8.98 (s, 1H), 8.83 (d, J=2.4 Hz, 1H), 8.68 (d, J=5.2 Hz, 1H), 8.24 (d, J=2.4 Hz, 1H), 8.12 (d, J=1.2 Hz, 1H), 7.92 (d, J=2.4 Hz, 1H), 7.89-7.87 (m, 1H), 7.62-7.60 (m, 1H), 7.35 (d, J=8.4 Hz, 1H), 7.90-7.89 (m, 1H), 6.62 (s, 1H), 6.05 (t, J=55.6 Hz, 1H), 2.86 (d, J=4.8 Hz, 3H), 2.29 (s, 3H), 1.22-1.19 (m, 4H). MS (ESI) m/z 460.0 [M+H]⁺

### Example 116. Synthesis of 4-(1-(difluoromethyl)cyclopropyl)-N-(6-methyl-5-(7-(methylamino)-1,6-naphthyridin-3-yl)pyridin-3-yl)picolinamide (Compound 128)

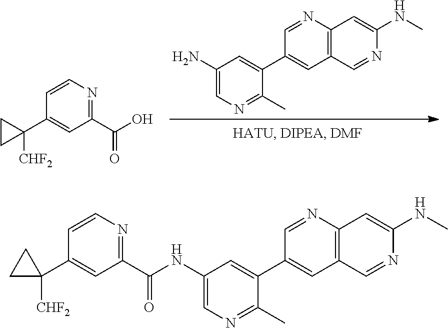 [SMILES: O=C(O)c1cc(C2(C(F)F)CC2)ccn1 ; CNc1cc2ncc(-c3cc(N)cnc3C)cc2cn1 ; CNc1cc2ncc(-c3cc(NC(=O)c4cc(C5(C(F)F)CC5)ccn4)cnc3C)cc2cn1]

A mixture of 4-(1-(difluoromethyl)cyclopropyl)picolinic acid (30 mg, 0.14 mmol), 3-(5-amino-2-methylpyridin-3-yl)-N-methyl-1,6-naphthyridin-7-amine (37 mg, 0.14 mmol), 1-[bis(dimethylamino)methylene]-1H-1,2,3-triazolo[4,5-b]pyridinium 3-oxid hexafluorophosphate (106 mg, 0.28 mmol), and N,N-diisopropylethylamine (54 mg, 0.42 mmol) in N,N-dimethylformamide (1 mL) was stirred at room temperature for 2 hours. The solution was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 4-(1-(difluoromethyl)cyclopropyl)-N-(6-methyl-5-(7-(methylamino)-1,6-naphthyridin-3-yl)pyridin-3-yl)picolinamide (18.3 mg, 0.04 mmol, 28%) as a yellow solid. ¹H NMR (400 MHz, DMSO-d₆) δ 10.98 (s, 1H), 9.05 (d, J=2.0 Hz, 1H), 8.99 (s, 1H), 8.86 (d, J=2.8 Hz, 1H), 8.71 (d, J=5.2 Hz, 1H), 8.33-8.30 (m, 2H), 8.14 (d, J=1.2 Hz, 1H), 7.64-7.62 (m, 1H), 6.99-6.96 (m, 1H), 6.63 (s, 1H), 6.06 (t, J=55.6 Hz, 1H), 2.87 (d, J=4.4 Hz, 3H), 2.51 (s, 3H), 1.32-1.20 (m, 4H). MS (ESI) m/z 461.1 [M+H]⁺

### Example 117. Synthesis of (S)—N-(4-methyl-3-(7-(methylamino)-1,6-naphthyridin-3-yl)phenyl)-5-(2,2,2-trifluoro-1-hydroxyethyl)nicotinamide and (R)—N-(4-methyl-3-(7-(methylamino)-1,6-naphthyridin-3-yl)phenyl)-5-(2,2,2-trifluoro-1-hydroxyethyl)nicotinamide (Compounds 129 and 130)

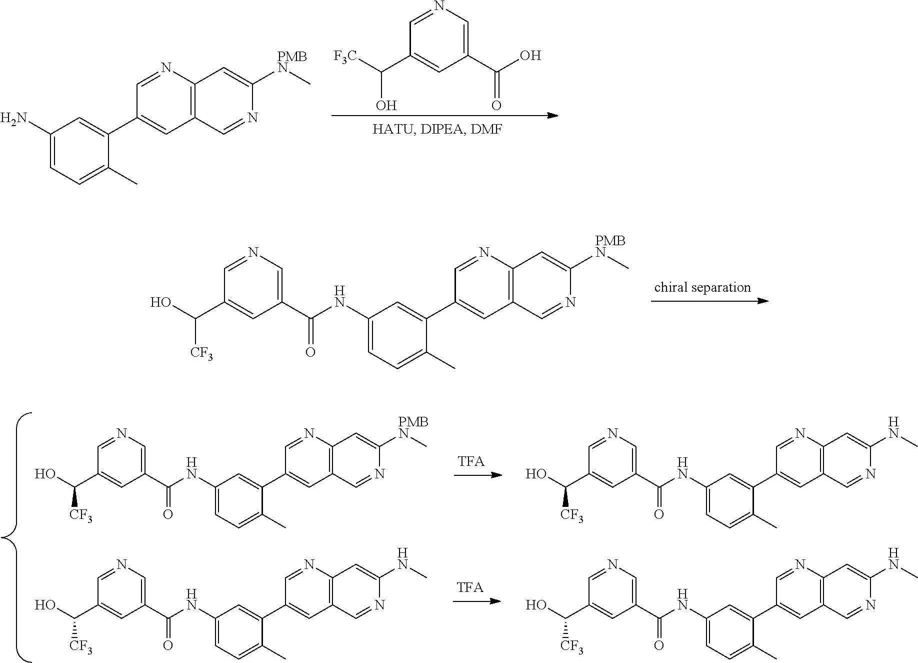

Step 1. A solution of 3-(5-amino-2-methylphenyl)-N-(4-methoxybenzyl)-N-methyl-1,6-naphthyridin-7-amine (100 mg, 0.26 mmol), 5-(2,2,2-trifluoro-1-hydroxyethyl)nicotinic acid (64 mg, 0.29 mmol), 1-[bis(dimethylamino)methylene]-1H-1,2,3-triazolo[4,5-b]pyridinium 3-oxid hexafluorophosphate (148 mg, 0.39 mmol) and N,N-diisopropylethylamine (134 mg, 1.04 mmol) in N,N-dimethylformamide (5 mL) was stirred at 25° C. for 3 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(3-(7-((4-methoxybenzyl)(methyl)amino)-1,6-naphthyridin-3-yl)-4-methylphenyl)-5-(2,2,2-trifluoro-1-hydroxyethyl)nicotinamide (90 mg, 0.15 mmol, 58%) as a yellow solid. MS (ESI) m/z 588.2 [M+H]⁺

Step 2. The enantiomers of N-(3-(7-((4-methoxybenzyl)(methyl)amino)-1,6-naphthyridin-3-yl)-4-methylphenyl)-5-(2,2,2-trifluoro-1-hydroxyethyl)nicotinamide (90 mg, 0.15 mmol) were separated by chiral SFC (Instrument: SFC-150 (Waters); Column: AS 20×250 mm, 10 μm (Daicel); Mobile phase: CO₂/MeOH (0.2% methanol ammonia)=55/45; Detection wavelength: 214 nm) to afford the first eluting enantiomer (retention time 5.3 min) which was arbitrarily assigned as (S)—N-(3-(7-((4-methoxybenzyl)(methyl)amino)-1,6-naphthyridin-3-yl)-4-methylphenyl)-5-(2,2,2-trifluoro-1-hydroxyethyl)nicotinamide (20.4 mg, 0.044 mmol) as a yellow solid. MS (ESI) m/z 588.2 [M+H]⁺;
and the second eluting enantiomer (retention time 6.8 min) which was arbitrarily assigned as (R)—N-(3-(7-((4-methoxybenzyl)(methyl)amino)-1,6-naphthyridin-3-yl)-4-methylphenyl)-5-(2,2,2-trifluoro-1-hydroxyethyl)nicotinamide (20.7 mg, 0.044 mmol) as a yellow solid. MS(ESI) m/z 588.2 [M+H]⁺

Step 3. A solution of (S)—N-(3-(7-((4-methoxybenzyl)(methyl)amino)-1,6-naphthyridin-3-yl)-4-methylphenyl)-5-(2,2,2-trifluoro-1-hydroxyethyl)nicotinamide (42 mg, 0.071 mmol) in trifluoroacetic acid (5 mL) was stirred at 25° C. for 3 hours. The reaction mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%-709% in 15 min) to afford (S)—N-(4-methyl-3-(7-(methylamino)-1,6-naphthyridin-3-yl)phenyl)-5-(2,2,2-trifluoro-1-hydroxyethyl)nicotinamide (20.4 mg, 0.044 mmol, 62%) as a yellow solid. ¹H NMR (400 MHz, DMSO-d₆) δ 10.59 (s, 1H), 9.17 (d, J=2.0 Hz, 1H), 8.99 (s, 1H), 8.86 (d, J=1.7 Hz, 1H), 8.82 (d, J=2.3 Hz, 1H), 8.42 (s, 1H), 8.24 (d, J=2.0 Hz, 1H), 7.80-7.72 (m, 2H), 7.37 (d, J=9.0 Hz, 1H), 7.23 (d, J=5.8 Hz, 1H), 6.91 (q, J=4.9 Hz, 1H), 6.63 (s, 1H), 5.51-5.42 (m, 1H), 2.87 (d, J=4.9 Hz, 3H), 2.29 (s, 3H). MS (ESI) m/z 468.1 [M+H]⁺

Step 4. A solution of (R)—N-(3-(7-((4-methoxybenzyl)(methyl)amino)-1,6-naphthyridin-3-yl)-4-methylphenyl)-5-(2,2,2-trifluoro-1-hydroxyethyl)nicotinamide (40 mg, 0.068 mmol) in 2,2,2-trifluoroacetic acid (5 mL) was stirred at 25° C. for 3 hours. The reaction mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%/6-70% in 15 min) to afford (R)—N-(4-methyl-3-(7-(methylamino)-1,6-naphthyridin-3-yl)phenyl)-5-(2,2,2-trifluoro-1-hydroxyethyl)nicotinamide (20.7 mg, 0.044 mmol, 65%) as a yellow solid. ¹H NMR (400 MHz, DMSO-de) δ 10.59 (s, 1H), 9.17 (s, 1H), 8.99 (s, 1H), 8.84 (d, J=13.9 Hz, 2H), 8.42 (s, 1H), 8.24 (s, 11H), 7.76 (s, 2H), 7.37 (d, J=8.9 Hz, 1H), 7.24 (d, J=5.7 Hz, 1H), 6.95-6.85 (m, 1H), 6.63 (s, 1H), 5.52-5.40 (m, 1H), 2.87 (d, J=4.8 Hz, 3H), 2.29 (s, 3H). MS (ESI) m/z 468.1 [M+H]⁺

### Example 118. Synthesis of (R)—N-(6-methyl-5-(7-(methylamino)-1,6-naphthyridin-3-yl)pyridin-3-yl)-4-(2,2,2-trifluoro-1-hydroxyethyl)picolinamide and (S)—N-(6-methyl-5-(7-(methylamino)-1,6-naphthyridin-3-yl)pyridin-3-yl)-4-(2,2,2-trifluoro-1-hydroxyethyl)picolinamide (Compounds 131 and 132)

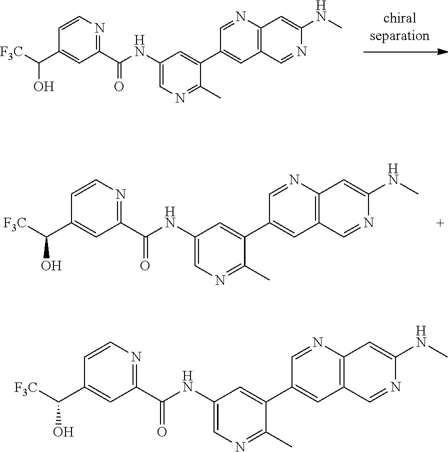 [SMILES: CNc1cc2ncc(-c3cc(NC(=O)c4cc(C(O)C(F)(F)F)ccn4)cnc3C)cc2cn1 ; CNc1cc2ncc(-c3cc(NC(=O)c4cc([C@@H](O)C(F)(F)F)ccn4)cnc3C)cc2cn1 ; CNc1cc2ncc(-c3cc(NC(=O)c4cc([C@H](O)C(F)(F)F)ccn4)cnc3C)cc2cn1]

The enantiomers of N-(6-methyl-5-(7-(methylamino)-1,6-naphthyridin-3-yl)pyridin-3-yl)-4-(2,2,2-trifluoro-1-hydroxyethyl)picolinamide (60 mg, 0.128 mmol) were separated by chiral SFC (Instrument: SFC-150 (Waters); Column: AS 20×250 mm, 10 μm (Daicel); Mobile phase: CO₂/MeOH (0.2% methanol ammonia)=50/50; Detection wavelength: 214 nm) to afford the first eluting enantiomer (retention time 2.3 min) which was arbitrarily assigned as (R)—N-(6-methyl-5-(7-(methylamino)-1,6-naphthyridin-3-yl)pyridin-3-yl)-4-(2,2,2-trifluoro-1-hydroxyethyl)picolinamide (15.1 mg, 0.032 mmol) as a yellow solid. ¹H NMR (400 MHz, DMSO-d₆) δ 11.02 (s, 1H), 9.04 (d, J=2.4 Hz, 1H), 8.99 (s, 1H), 8.87 (d, J=2.3 Hz, 1H), 8.83 (d, J=5.1 Hz, 1H), 8.32 (dd, J=5.4, 2.2 Hz, 3H), 7.82 (d, J=4.6 Hz, 1H), 6.97 (d, J=5.0 Hz, 1H), 6.64 (s, 1H), 5.52 (t, J=7.4 Hz, 1H), 2.88 (d, J=5.0 Hz, 3H), 2.51 (s, 3H). MS (ESI) m/z 469.0 [M+H]⁺;
and the second eluting enantiomer (retention time 3.4 min) which was arbitrarily assigned as (S)—N-(6-methyl-5-(7-(methylamino)-1,6-naphthyridin-3-yl)pyridin-3-yl)-4-(2,2,2-trifluoro-1-hydroxyethyl)picolinamide (16.7 mg, 0.035 mmol) as a yellow solid. ¹H NMR (400 MHz, DMSO-d₆) δ 11.02 (s, 1H), 9.04 (d, J=2.4 Hz, 1H), 8.99 (s, 1H), 8.87 (d, J=2.3 Hz, 1H), 8.83 (d, J=5.1 Hz, 1H), 8.32 (dd, J=5.4, 2.2 Hz, 3H), 7.82 (d, J=4.6 Hz, 1H), 6.97 (d, J=5.0 Hz, 1H), 6.64 (s, 1H), 5.52 (t, J=7.4 Hz, 1H), 2.88 (d, J=5.0 Hz, 3H), 2.51 (s, 3H). MS (ESI) m/z 469.1 [M+H]⁺

### Example 119. Synthesis of N-(4,6-dimethyl-5-(7-(methylamino)-1,6-naphthyridin-3-yl)pyridin-3-yl)-4-(trifluoromethyl)picolinamide (Compound 133)

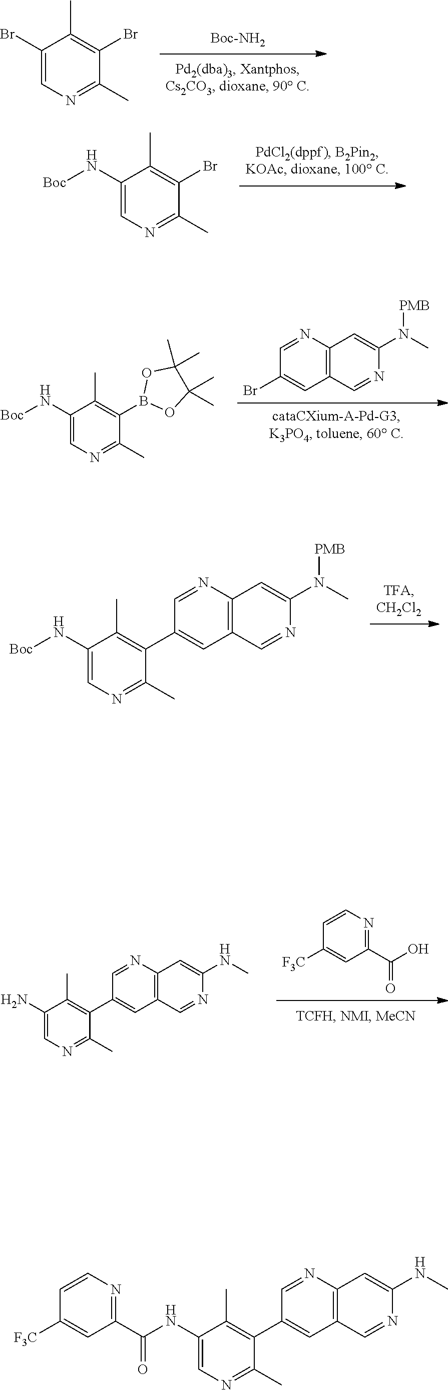

Step 1. A mixture of 3,5-dibromo-2,4-dimethyl-pyridine (2 g, 7.6 mmol), tert-butyl carbamate (0.89 g, 7.6 mmol), tris(dibenzylideneacetone)dipalladium (0.69 g, 0.75 mmol), 4,5-bis(diphenylphosphino)-9,9-dimethylxanthene (Xantphos, 0.87 g, 1.5 mmol), and cesium carbonate (4.9 g, 15 mmol) in dioxane (30 mL) was stirred at 90° C. under nitrogen for 16 hours. On completion, the mixture was concentrated to give a residue. The residue was purified by flash chromatography (silica, petroleum ether/ethyl acetate=I/O to 3/1) to give tert-butyl (5-bromo-4,6-dimethylpyridin-3-yl)carbamate (1.8 g, 79%6) as a yellow oil. ¹H NMR (400 MHz, DMSO-d₆) δ 8.98 (s, 1H), 8.26 (s, 1H), 2.57 (s, 3H), 2.28 (s, 3H), 1.46 (s, 9H). MS (ESI) m/z 301.3 [M+H]⁺

Step 2. To a solution of tert-butyl (5-bromo-4,6-dimethylpyridin-3-yl)carbamate (1.6 g, 5.3 mmol) and 4,4,5,5-tetramethyl-2-(4,4,5,5-tetramethyl-1,3,2-dioxaborolan-2-yl)-1,3,2-dioxaborolane (2 g, 8 mmol) in dioxane (18 mL) was added 1,1′-bis(diphenylphosphino)ferrocene-palladium(II)dichloride (0.39 g, 0.53 mmol) and potassium acetate (0.52 g, 5.3 mmol). The mixture was stirred at 100° C. for 2 hours. The mixture was filtered, and the filter cake was concentrated in vacuo to give a residue. The residue was purified by flash chromatography (silica, petroleum ether/ethyl acetate=5/1 to 3/1) to give ter-butyl (4,6-dimethyl-5-(4,4,5,5-tetramethyl-1,3,2-dioxaborolan-2-yl)pyridin-3-yl)carbamate (0.35 g, 9.7%) as a white solid. ¹H NMR (400 MHz, DMSO-d₆) δ 7.07 (s, 1H), 2.43 (s, 3H), 2.37 (s, 3H), 1.44 (d, J=1.5 Hz, 12H), 1.34 (s, 9H). MS (ESI) m/z 349.1 [M+H]⁺

Step 3. To a solution of 3-bromo-N-[(4-methoxyphenyl)methyl]-N-methyl-1,6-naphthyridin-7-amine (0.16 g, 0.45 mmol) and tert-butyl (4,6-dimethyl-5-(4,4,5,5-tetramethyl-1,3,2-dioxaborolan-2-yl)pyridin-3-yl)carbamate (0.28 g, 0.8 mmol) in toluene (1.6 mL) was added aqueous potassium phosphate tribasic (1.5 M, 0.89 mL) and [(di(1-adamantyl)-butylphosphine)-2-(2′-amino-1,1′-biphenyl)]palladium(II) methanesulfonate (cataCXium-A-Pd-G3, 33 mg, 0.045 mmol). The mixture was stirred at 60° C. for 16 hours. The mixture was poured into water (20 mL) and then extracted with ethyl acetate (20 mL×2). The combined organic phases were concentrated in vacuo to give a residue. The residue was purified by flash chromatography (silica, petroleum ether/ethyl acetate=5/1 to 1/1) to give tert-butyl (5-(7-((4-methoxybenzyl)(methyl)amino)-1,6-naphthyridin-3-yl)-4,6-dimethylpyridin-3-yl)carbamate (186 mg, 83%) as a yellow oil. MS (ESI) m/z 500.0 [M+H]⁺

Step 4. To a solution of tert-butyl (5-(7-((4-methoxybenzyl)(methyl)amino)-1,6-naphthyridin-3-yl)-4,6-dimethylpyridin-3-yl)carbamate (0.19 g, 0.37 mmol) in dichloromethane (3 mL) was added trifluoroacetic acid (42 mg, 0.37 mmol). The mixture was stirred at 25° C. for 1 hour. The mixture was concentrated in vacuo to give 3-(5-amino-2,4-dimethylpyridin-3-yl)-N-methyl-1,6-naphthyridin-7-amine (112 mg, crude, TFA salt) as a white solid. MS (ESI) m/z 280.2 [M+H]⁺

Step 5. To a solution of 3-(5-amino-2,4-dimethyl-3-pyridyl)-N-methyl-1,6-naphthyridin-7-amine (0.11 g, 0.28 mmol) and 4-(trifluoromethyl)pyridine-2-carboxylic acid (54 mg, 0.28 mmol) in acetonitrile (2.5 mL) was added chloro-N,N,N′,N′-tetramethylformamidinium hexafluorophosphate (TCFH, 0.16 g, 0.57 mmol) and 1-methylimidazole (93 mg, 1.1 mmol). The mixture was stirred at 25° C. for 2 hours. The mixture was concentrated and the residue was purified by prep-HPLC (Column: Waters Xbridge BEH 25×150 mm C18, 5 μm, Mobile Phase A: water (10 mM ammonium bicarbonate), B: acetonitrile) to give N-(4,6-dimethyl-5-(7-(methylamino)-1,6-naphthyridin-3-yl)pyridin-3-yl)-4-(trifluoromethyl)picolinamide (33 mg, 25%) as a white solid. ¹H NMR (400 MHz, DMSO-d₆) δ 10.70 (s, 1H), 9.05 (d, J=5.2 Hz, 1H), 8.96 (s, 1H), 8.64 (d, J=2.0 Hz, 1H), 8.58 (s, 1H), 8.35 (s, 1H), 8.16 (d, J=2.0 Hz, 1H), 8.12 (d, J=4.4 Hz, 1H), 6.92 (q, J=4.8 Hz, 1H), 6.63 (s, 1H), 2.87 (d, J=4.8 Hz, 3H), 2.27 (s, 3H), 2.00 (s, 3H). MS (ESI) m/z 452.9 [M+H]⁺

### Example 120. Synthesis of 3,3-dimethyl-N-(6-methyl-5-(7-(methylamino)-1,6-naphthyridin-3-yl)pyridin-3-yl)-2,3-dihydrofuro[3,2-b]pyridine-7-carboxamide (Compound 134)

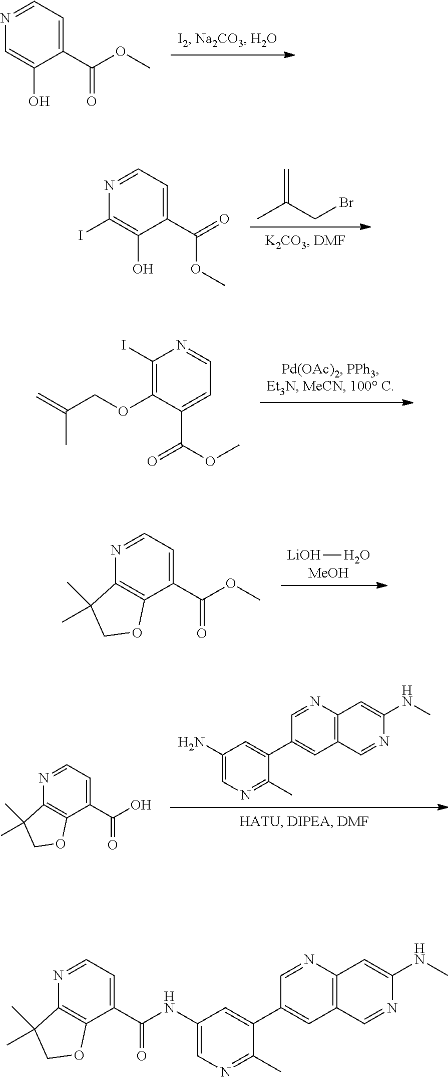

Step 1. A mixture of methyl 3-hydroxyisonicotinate (3.06 g, 20 mmol), iodine (5.58 g, 22 mmol) and sodium carbonate (2.33 g, 22 mmol) in water (50 mL) was stirred at 20° C. for 2 hours. The reaction solution was extracted with ethyl acetate (50 mL×3). The combined organic phases were concentrated and purified by flash chromatography (silica, petroleum ether/ethyl acetate=10/1) to give methyl 3-hydroxy-2-iodoisonicotinate (1.8 g, 6.45 mmol, 32%) as a white solid. MS (ESI) m/z 280.0 [M+H]⁺

Step 2. A mixture of methyl 3-hydroxy-2-iodoisonicotinate (1.8 g, 6.45 mmol), 3-bromo-2-methylprop-1-ene (1.05 g, 7.74 mmol) and potassium carbonate (2.67 g, 19.35 mmol) in N,N-dimethylformamide (30 mL) was stirred at 25° C. for 3 hours. The mixture was diluted with ethyl acetate (100 mL) and washed with water (100 mL×3). The organic phase was washed with brine (40 mL), dried over sodium sulfate, filtered, and concentrated. The residue was purified by flash chromatography (silica, petroleum ether/ethyl acetate=10/1) to give methyl 2-iodo-3-((2-methylallyl)oxy)isonicotinate (2.0 g, 6.0 mmol, 93%) as a colorless oil. MS (ESI) m/z 334.1 [M+H]⁺

Step 3. A mixture of methyl 2-iodo-3-((2-methylallyl)oxy)isonicotinate (0.66 g, 2.0 mmol), palladium (II) acetate (133 mg, 0.6 mmol), triethylamine (200 mg, 2.0 mmol) and triphenylphosphine (155 mg, 0.6 mmol) in acetonitrile (2 mL) was stirred at 100° C. for 6 hours under nitrogen atmosphere. After cooling to room temperature, the reaction was diluted with water (20 mL) and extracted with ethyl acetate (30 mL×2). The combined organic phases were washed with brine (30 mL), dried over sodium sulfate, filtered, and concentrated. The crude residue was purified by flash chromatography (silica, petroleum ether/ethyl acetate=9/1) to give methyl 3,3-dimethyl-2,3-dihydrofuro[3,2-b]pyridine-7-carboxylate (90 mg, 0.43 mmol, 22%) as a yellow solid. MS (ESI) m/z 208.2 [M+H]⁺

Step 4. To a solution of methyl 3,3-dimethyl-2,3-dihydrofuro[3,2-b]pyridine-7-carboxylate (90 mg, 0.43 mmol) in methanol (5 mL) was added lithium hydroxide monohydrate (91 mg, 2.15 mmol) in water (1 mL). After stirring at 25° C. for 1 hour, the mixture was evaporated under reduced pressure to give 3,3-dimethyl-2,3-dihydrofuro[3,2-b]pyridine-7-carboxylic acid (80 mg, crude) as a white solid. MS (ESI) m/z 194.1 [M+H]⁺

Step 5. A solution of 3-(5-amino-2-methylpyridin-3-yl)-N-methyl-1,6-naphthyridin-7-amine (55 mg, 0.2 mmol), 3,3-dimethyl-2,3-dihydrofuro[3,2-b]pyridine-7-carboxylic acid (80 mg, crude), 1-[bis(dimethylamino)methylene]-1H-1,2,3-triazolo[4,5-b]pyridinium 3-oxid hexafluorophosphate (236 mg, 0.6 mmol), and N,N-diisopropylethylamine (160 mg, 1.2 mmol) in N,N-dimethylformamide (10 mL) was stirred at 25° C. for 4 hours. The reaction was diluted with water (20 mL) and extracted with ethyl acetate (30 mL×2). The combined organic phases were washed with brine (30 mL), dried over sodium sulfate, filtered, and concentrated. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3,3-dimethyl-N-(6-methyl-5-(7-(methylamino)-1,6-naphthyridin-3-yl)pyridin-3-yl)-2,3-dihydrofuro[3,2-b]pyridine-7-carboxamide (18.4 mg, 0.042 mmol, 21%) as a yellow solid. ¹H NMR (400 MHz, DMSO-d₆) δ 10.17 (s, 1H), 8.99 (s, 1H), 8.85 (s, 2H), 8.32 (s, 1H), 8.18 (d, J=5.2 Hz, 1H), 8.12 (s, 1H), 7.46 (d, J=5.1 Hz, 1H), 6.95 (d, J=5.1 Hz, 1H), 6.63 (s, 1H), 4.55 (s, 2H), 2.88 (d, J=4.8 Hz, 3H), 2.48 (s, 3H), 1.36 (s, 6H). MS (ESI) m/z 441.1 [M+H]⁺

### Example 121. Synthesis of (R)—N-(6-methyl-5-(7-(methylamino)-1,6-naphthyridin-3-yl)pyridin-3-yl)-2-(2,2,2-trifluoro-1-methoxyethyl)isonicotinamide and (S)—N-(6-methyl-5-(7-(methylamino)-1,6-naphthyridin-3-yl)pyridin-3-yl)-2-(2,2,2-trifluoro-1-methoxyethyl)isonicotinamide (Compounds 135 and 136)

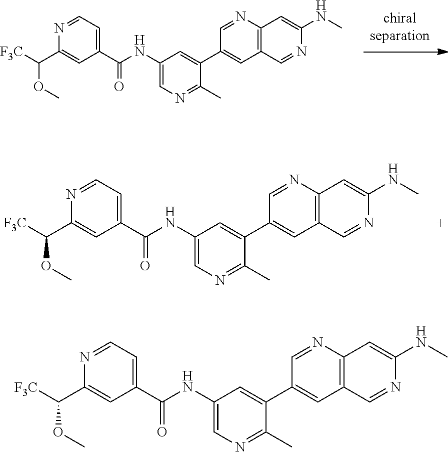 [SMILES: CNc1cc2ncc(-c3cc(NC(=O)c4ccnc(C(OC)C(F)(F)F)c4)cnc3C)cc2cn1 ; CNc1cc2ncc(-c3cc(NC(=O)c4ccnc([C@@H](OC)C(F)(F)F)c4)cnc3C)cc2cn1 ; CNc1cc2ncc(-c3cc(NC(=O)c4ccnc([C@H](OC)C(F)(F)F)c4)cnc3C)cc2cn1]

The enantiomers of N-(6-methyl-5-(7-(methylamino)-1,6-naphthyridin-3-yl)pyridin-3-yl)-2-(2,2,2-trifluoro-1-methoxyethyl)isonicotinamide (50 mg, 0.1 mmol) were separated by chiral SFC (Instrument: SFC-150 (Waters): Column: AD 20×250 mm, 10 μm (Daicel); Mobile phase: CO₂/MeOH (0.2% methanol ammonia)=65/35; Detection wavelength: 214 nm) to afford the first eluting enantiomer (retention time 5.0 min) which was arbitrarily assigned as (R)—N-(6-methyl-5-(7-(methylamino)-1,6-naphthyridin-3-yl)pyridin-3-yl)-2-(2,2,2-trifluoro-1-methoxyethyl)isonicotinamide (15.5 mg, 0.032 mmol) as a yellow solid. ¹H NMR (400 MHz, DMSO-d₆) δ 2.50 (s, 3H), 2.87 (d, J=4.8 Hz, 3H), 3.47 (s, 3H), 5.20 (q, J=6.8 Hz, 1H), 6.64 (s, 1H), 6.94 (q, J=4.4 Hz, 1H), 8.02 (d, J=5.2 Hz, 1H), 8.08 (s, 1H), 8.16 (d, J=2.0 Hz, 1H), 8.31 (d, J=1.6 Hz, 1H), 8.86-8.90 (m, 3H), 8.99 (s, 1H), 10.85 (s, 1H). MS (ESI) m/z 483.1 [M+H]⁺; and the second eluting enantiomer (retention time 6.3 min) which was arbitrarily assigned as (S)—N-(6-methyl-5-(7-(methylamino)-1,6-naphthyridin-3-yl)pyridin-3-yl)-2-(2,2,2-trifluoro-1-methoxyethyl)isonicotinamide (2.4 mg, 0.0050 mmol) as a yellow solid. ¹H NMR (400 MHz, DMSO-d₆) δ 2.50 (s, 3H), 2.87 (d, J=4.8 Hz, 3H), 3.47 (s, 3H), 5.20 (q, J=6.8 Hz, 1H), 6.63 (s, 1H), 6.94 (q, J=4.8 Hz, 1H), 8.02 (d, J=5.2 Hz, 1H), 8.07 (s, 1H), 8.15 (d, J=2.0 Hz, 1H), 8.31 (d, J=1.6 Hz, 1H), 8.86-8.89 (m, 3H), 8.99 (s, 1H), 10.84 (s, 1H). MS (EST) m/z 483.1 [M+H]⁺

### Example 122. Synthesis of 3-(5-((1-(2,2-difluoroethyl)-1H-pyrazol-4-yl)amino)-2-methylpyridin-3-yl)-N-methyl-1,6-naphthyridin-7-amine (Compound 137)

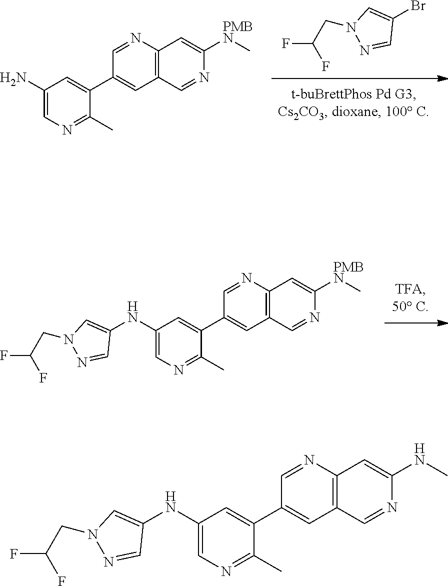

Step 1. A mixture of 4-bromo-1-(2,2-difluoroethyl)-1H-pyrazole (60 mg, 0.285 mmol), 3-(5-amino-2-methylpyridin-3-yl)-N-(4-methoxybenzyl)-N-methyl-1,6-naphthyridin-7-amine (100 mg, 0.26 mmol), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (22 mg, 0.026 mmol) and cesium carbonate (254 mg, 0.78 mmol) in 1,4-dioxane (2 mL) was stirred at 100° C. for 16 hours under nitrogen atmosphere. After cooling to room temperature, the reaction was diluted with water (20 mL) and extracted with ethyl acetate (20 mL×2). The combined organic phases were washed with brine (30 mL), dried over sodium sulfate, filtered, and concentrated. The crude residue was purified by flash chromatography (silica, methanol/dichloromethane=1/10) to give 3-(5-((1-(2,2-difluoroethyl)-1H-pyrazol-4-yl)amino)-2-methylpyridin-3-yl)-N-(4-methoxybenzyl)-N-methyl-1,6-naphthyridin-7-amine (100 mg, 0.194 mmol, 74%) as a yellow solid. MS (ESI) m/z 516.2 [M+H]⁺

Step 2. A solution of 3-(5-((1-(2,2-difluoroethyl)-1H-pyrazol-4-yl)amino)-2-methylpyridin-3-yl)-N-(4-methoxybenzyl)-N-methyl-1,6-naphthyridin-7-amine (100 mg, 0.194 mmol) in trifluoroacetic acid (10 mL) was stirred at 50° C. for 1 hour. The mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-(5-((1-(2,2-difluoroethyl)-1H-pyrazol-4-yl)amino)-2-methylpyridin-3-yl)-N-methyl-1,6-naphthyridin-7-amine (52.6 mg, 0.133 mmol, 68%) as a yellow solid. ¹H NMR (400 MHz, DMSO-d₆) δ 8.96 (s, 1H), 8.81 (s, 1H), 8.24 (s, 1H), 8.11 (s, 1H), 7.85 (d, J=25.7 Hz, 2H), 7.50 (s, 1H), 7.07 (s, 1H), 6.90 (d, J=4.7 Hz, 1H), 6.62 (s, 1H), 6.34 (t, J=55.2 Hz, 1H), 4.56 (dd, J=20.7, 9.0 Hz, 2H), 2.87 (d, J=4.7 Hz, 3H), 2.35 (s, 3H). MS (ESI) m/z 396.1 [M+H]⁺

### Example 123. Synthesis of 3-(5-((1-isopropyl-1H-pyrazol-4-yl)amino)-2-methylpyridin-3-yl)-N-methyl-1,6-naphthyridin-7-amine (Compound 138)

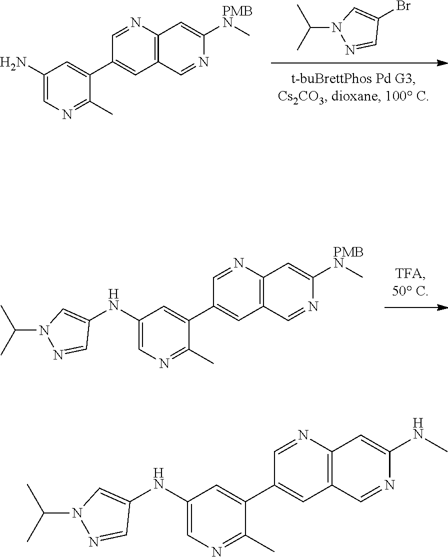

Step 1. A solution of 4-bromo-1-isopropyl-1H-pyrazole (110 mg, 0.58 mmol), 3-(5-amino-2-methylpyridin-3-yl)-N-(4-methoxybenzyl)-N-methyl-1,6-naphthyridin-7-amine (224 mg, 0.58 mmol), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (50 mg, 0.06 mmol) and cesium carbonate (567 mg, 1.74 mmol) in 1,4-dioxane (2 mL) was stirred at 100° C. overnight under nitrogen atmosphere. After cooling to room temperature, the reaction was poured into water (20 mL) and extracted with ethyl acetate (20 mL×3). The combined organic phases were concentrated and purified by flash chromatography (silica, petroleum ether/ethyl acetate=4/1) to give 3-(5-((1-isopropyl-1H-pyrazol-4-yl)amino)-2-methylpyridin-3-yl)-N-(4-methoxybenzyl)-N-methyl-1,6-naphthyridin-7-amine (200 mg, 0.40 mmol, 70%) as a yellow solid. MS (ESI) m/z 494.2 [M+H]⁺

Step 2. A solution of 3-(5-((1-isopropyl-1H-pyrazol-4-yl)amino)-2-methylpyridin-3-yl)-N-(4-methoxybenzyl)-N-methyl-1,6-naphthyridin-7-amine (200 mg, 0.40 mmol) in trifluoroacetic acid (4 mL) was stirred at 50° C. overnight. After cooling to room temperature, the reaction mixture was adjusted to pH=8.0 with saturated aqueous sodium bicarbonate solution. The mixture was concentrated and dissolved in dimethyl sulfoxide (1 mL). The solution was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-(5-((1-isopropyl-1H-pyrazol-4-yl)amino)-2-methylpyridin-3-yl)-N-methyl-1,6-naphthyridin-7-amine (94.4 mg, 0.25 mmol, 63%) as a yellow solid. ¹H NMR (400 MHz, DMSO-d₆) δ 8.95 (s, 1H), 8.79 (d, J=2.4 Hz, 1H), 8.23 (d, J=2.0 Hz, 1H), 8.07 (d, J=2.8 Hz, 1H), 7.76 (s, 1H), 7.73 (s, 1H), 7.38 (d, J=0.8 Hz, 1H), 7.03 (d, J=2.8 Hz, 1H), 6.92-6.89 (m, 1H), 6.60 (s, 1H), 4.46-4.39 (m, 1H), 2.85 (d, J=4.8 Hz, 3H), 2.34 (s, 3H), 1.38 (d, J=6.4 Hz, 6H). MS (ESI) m/z 374.2 [M+H]⁺

### Example 124. Synthesis of 3-(5-((1-(2,2-difluoroethyl)-1H-imidazol-4-yl)amino)-2-methylpyridin-3-yl)-N-methyl-1,6-naphthyridin-7-amine (Compound 139)

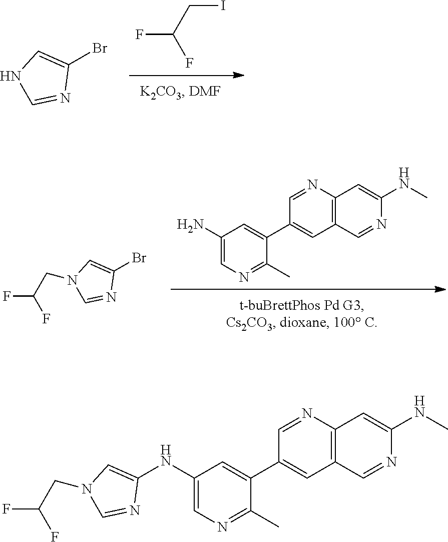

Step 1. A solution of 4-bromo-1H-imidazole (500 mg, 3.40 mmol), 1,1-difluoro-2-iodoethane (653 mg, 3.40 mmol) and potassium carbonate (1.41 g, 10.2 mmol) in N,N-dimethylformamide (5 mL) was stirred at room temperature for 16 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-bromo-1-(2,2-difluoroethyl)-1H-imidazole (152 mg, 0.72 mmol, 21%). MS (ESI) m/z 212.0 [M+H]⁺

Step 2. A solution of 4-bromo-1-(2,2-difluoroethyl)-1H-imidazole (30 mg, 0.14 mmol), 3-(5-amino-2-methylpyridin-3-yl)-N-methyl-1,6-naphthyridin-7-amine (37 mg, 0.14 mmol), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (1.2 mg, 0.014 mmol) and cesium carbonate (137 mg, 0.42 mmol) in 1,4-dioxane (3 mL) was stirred at 100° C. for 16 hours. After cooling to room temperature, the reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3-(5-((1-(2,2-difluoroethyl)-1H-imidazol-4-yl)amino)-2-methylpyridin-3-yl)-N-methyl-1,6-naphthyridin-7-amine (21.6 mg, 0.05 mmol, 39%). ¹H NMR (500 MHz, DMSO-d₆) δ 8.97 (s, 1H), 8.79 (d, J=2.3 Hz, 1H), 8.34 (s, 1H), 8.27 (d, J=2.6 Hz, 1H), 8.23 (d, J=2.3 Hz, 1H), 7.48 (s, 1H), 7.42 (d, J=2.6 Hz, 1H), 6.92-6.87 (m, 2H), 6.62 (s, 1H), 6.45-6.21 (m, 1H), 4.43 (td, J=15.7, 3.6 Hz, 2H), 2.87 (d, J=4.9 Hz, 3H), 2.35 (s, 3H). MS (ESI)m m/z 396.1 [M+H]⁺

### Example 125. Synthesis of 4-(2,2-difluoro-1-hydroxypropyl)-N-(6-methyl-5-(7-(methylamino)-1,6-naphthyridin-3-yl)pyridin-3-yl)picolinamide (Compound 140)

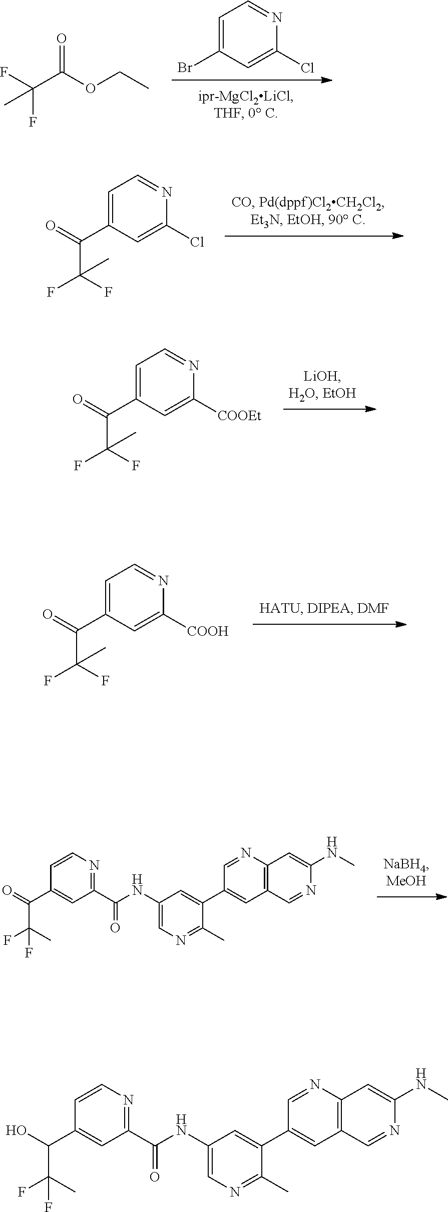

Step 1. Isopropylmagnesium chloride-lithium chloride complex (15.7 ml, 20.4 mmol) was added to a solution of 4-bromo-2-chloropyridine (2000 mg, 20.4 mmol) in tetrahydrofuran (20 mL) at 0° C. After stirring at 0° C. for 1 hour under nitrogen, ethyl 2,2-difluoropropanoate (3097 mg, 22.4 mmol) was added. The reaction solution was concentrated and the residue was purified by flash chromatography (silica, dichloromethane/methanol=20/1) to give 1-(2-chloropyridin-4-yl)-2,2-difluoropropan-1-one (100 mg, 0.49 mmol, 2.4%) as a white solid. MS (ESI) m/z 206.1 [M+H]⁺

Step 2. A solution of 1-(2-chloropyridin-4-yl)-2,2-difluoropropan-1-one (100 mg, 0.48 mmol), 1,1′-bis(diphenylphosphino)ferrocene-palladium(II)dichloride dichloromethane complex (42 mg, 0.05 mmol) and triethylamine (339 mg, 3.36 mmol) in ethanol (15 mL) was stirred at 90° C. for 16 hours under carbon monoxide atmosphere. After cooling to room temperature, the reaction solution was concentrated to give ethyl 4-(2,2-difluoropropanoyl)picolinate (crude) as a brown oil, which was used without further purification. MS (ESI) m/z 244.1 [M+H]⁺

Step 3. A solution of ethyl 4-(2,2-difluoropropanoyl)picolinate (crude) and sodium hydroxide (58 mg, 1.43 mmol) in methanol (3 mL) and water (2 mL) was stirred for 2 hours at room temperature. The solvent was evaporated under reduced pressure. The residue was diluted with water (5 mL), adjusted to pH=3 by adding 1 N hydrochloric acid slowly and extracted with ethyl acetate (50 mL×3). The combined organic layers were concentrated in vacuo to give 4-(2,2-difluoropropanoyl)picolinic acid (30 mg, 0.14 mmol, 29%/c) as a yellow solid. MS (ESI) m/z 234.1 [M+H+18]⁺

Step 4. A solution of 4-(2,2-difluoropropanoyl)picolinic acid (30 mg, 0.14 mmol), 3-(5-amino-2-methylpyridin-3-yl)-N-methyl-1,6-naphthyridin-7-amine (45 mg, 0.10 mmol), 1-[bis(dimethylamino)methylene]-1H-1,2,3-triazolo[4,5-b]pyridinium 3-oxid hexafluorophosphate (114 mg, 0.30 mmol) and N,N-diisopropylethylamine (39 mg, 0.30 mmol) in N,N-dimethylformamide (3 mL) was stirred at room temperature for 2 hours. The crude residue was purified by flash chromatography (silica, petroleum ether/ethyl acetate=1/3) to give 4-(2,2-difluoropropanoyl)-N-(6-methyl-5-(7-(methylamino)-1,6-naphthyridin-3-yl)pyridin-3-yl)picolinamide (18 mg, 0.04 mmol, 29%)%) as a yellow solid. MS (ESI) m/z 481.0 [M+H+18]

Step 5. To a solution of 4-(2,2-difluoropropanoyl)-N-(6-methyl-5-(7-(methylamino)-1,6-naphthyridin-3-yl)pyridin-3-yl)picolinamide (18 mg, 0.04 mmol) in methanol (3 mL) was added sodium borohydride (1634 mg, 0.12 mmol) at room temperature. After stirring at room temperature for 4 hours, the reaction mixture was quenched by water (6 mL) and extracted with ethyl acetate (10 mL×2). The combined organic phases were concentrated and purified by flash chromatography (silica, petroleum ether/ethyl acetate=3/1) to give 4-(2,2-difluoro-1-hydroxypropyl)-N-(6-methyl-5-(7-(methylamino)-1,6-naphthyridin-3-yl)pyridin-3-yl)picolinamide (5 mg, 0.01 mmol, 25%) as a yellow solid. ¹H NMR (400 MHz, DMSO-d₆) δ 10.99 (s, 1H), 9.05 (d, J=2.2 Hz, 1H), 8.99 (s, 1H), 8.87 (d, J=2.1 Hz, 1H), 8.76 (d, J=5.0 Hz, 1H), 8.32 (d, J=2.2 Hz, 2H), 8.24 (s, 1H), 7.72 (d, J=4.7 Hz, 1H), 6.97 (d, J=5.0 Hz, 1H), 6.70 (d, J=5.5 Hz, 1H), 6.64 (s, 1H), 5.01 (dd, J=13.6, 6.1 Hz, 1H), 2.88 (d, J=4.9 Hz, 3H), 2.50 (d, J=1.9 Hz, 3H), 1.59 (t, J=19.1 Hz, 3H). MS (ESI) m/z 465.1 [M+H]⁺

### Example 126. Synthesis of (R)—N-(6-methyl-5-(7-(methylamino)-1,6-naphthyridin-3-yl)pyridin-3-yl)-4-(2,2,2-trifluoro-1-methoxyethyl)picolinamide and (S)—N-(6-methyl-5-(7-(methylamino)-1,6-naphthyridin-3-yl)pyridin-3-yl)-4-(2,2,2-trifluoro-1-methoxyethyl)picolinamide (Compounds 141 and 142)

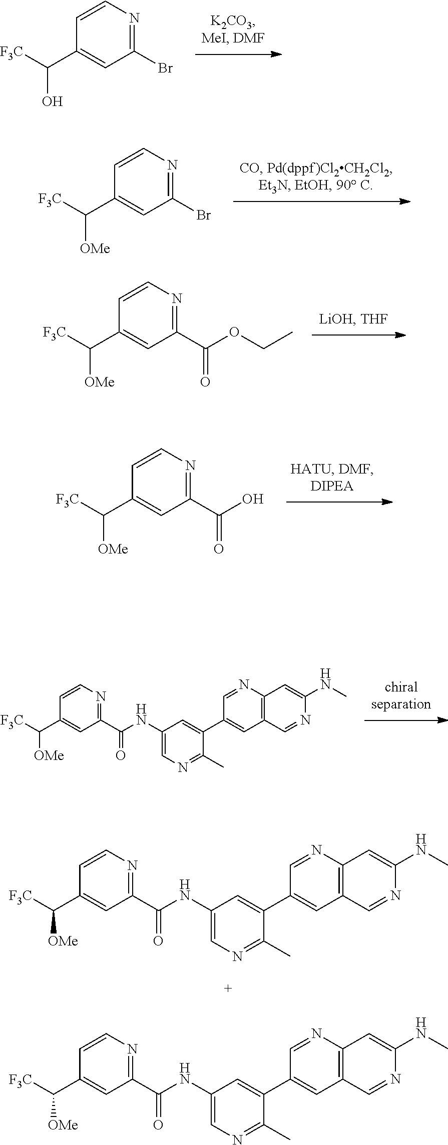

Step 1. A solution of 1-(2-bromopyridin-4-yl)-2,2,2-trifluoroethan-1-ol (2000 mg, 7.81 mmol) and iodomethane (3327 mg, 23.43 mmol) and potassium carbonate (3233 mg, 23.43 mmol) in acetonitrile (10 mL) was stirred at 25° C. for 16 hours. The reaction mixture was quenched with ice water (40 mL) and extracted with ethyl acetate (30 mL×2). The combined organic phases were concentrated and purified by flash chromatography (silica, petroleum ether/ethyl acetate=1/1) to give 2-bromo-4-(2,2,2-trifluoro-1-methoxyethyl)pyridine (1500 mg, 5.58 mmol, 71%) as a yellow oil. MS (ESI) m/z 270.0 [M+H]⁺

Step 2. A solution of 2-bromo-4-(2,2,2-trifluoro-1-methoxyethyl)pyridine (300 mg, 1.12 mmol), 1,1′-bis(diphenylphosphino)ferrocene-palladium(II)dichloride dichloromethane complex (90 mg, 0.11 mmol) and triethylamine (339 mg, 3.36 mmol) in ethanol (15 mL) was stirred at 90° C. for 16 hours under carbon monoxide atmosphere. The reaction was cooled to room temperature and concentrated. The residue was quenched with water (15 mL) and extracted with ethyl acetate (30 mL×2). The combined organic phases were concentrated and purified by flash chromatography (silica, petroleum ether/ethyl acetate=1/1) to give ethyl 4-(2,2,2-trifluoro-1-methoxyethyl)picolinate (200 mg, 0.76 mmol, 69%) as a brown oil. MS (ESI) m/z 264.1 [M+H]⁺

Step 3. The mixture of ethyl 4-(2,2,2-trifluoro-1-methoxyethyl)picolinate (200 mg, 0.76 mmol) and lithium hydroxide (64 mg, 1.52 mmol) in tetrahydrofuran (5 mL) was stirred at room temperature for 1 hour. The reaction mixture was extracted with ethyl acetate (30 mL×2). The water layer was adjusted to pH=4 with hydrochloric acid (6 N) and extracted with ethyl acetate (30 mL×2). The combined organic phases were concentrated to give 4-(2,2,2-trifluoro-1-methoxyethyl)picolinic acid (150 mg, 0.77 mmol, 64%) as a brown oil. MS (ESI) m/z 236.1 [M+H]⁺

Step 4. A mixture of 4-(2,2,2-trifluoro-1-methoxyethyl)picolinic acid (100 mg, 0.50 mmol), 3-(5-amino-2-methylphenyl)-N-methyl-1,6-naphthyridin-7-amine (132 mg, 0.50 mmol), 1-[bis(dimethylamino)methylene]-1H-1,2,3-triazolo[4,5-b]pyridinium 3-oxid hexafluorophosphate (208 mg, 0.54 mmol) and N,N-diisopropylethylamine (128 mg, 1.0 mmol) in N,N-dimethylformamide (6 mL) was stirred at room temperature for 1 hour. The reaction mixture was treated with water (50 mL). The precipitate was filtered, washed with water (5 mL) and dried to give N-(6-methyl-5-(7-(methylamino)-1,6-naphthyridin-3-yl)pyridin-3-yl)-4-(2,2,2-trifluoro-1-methoxyethyl)picolinamide (102 mg, 0.21 mmol, 41%) as a yellow solid. MS (EST) m/z 483.0 [M+H]⁺

Step 5. The enantiomers of N-(6-methyl-5-(7-(methylamino)-1,6-naphthyridin-3-yl)pyridin-3-yl)-4-(2,2,2-trifluoro-1-methoxyethyl)picolinamide (102 mg, 0.21 mmol) were separated by chiral SFC (Instrument: SFC-150 (Waters); Column: OJ 20×250 mm, 10 μm (Daicel); Mobile phase: CO₂/MeOH (0.2% methanol ammonia)=40/60; Detection wavelength: 214 nm) to afford the first eluting enantiomer (retention time 2.1 min) which was arbitrarily assigned as (R)—N-(6-methyl-5-(7-(methylamino)-1,6-naphthyridin-3-yl)pyridin-3-yl)-4-(2,2,2-trifluoro-1-methoxyethyl)picolinamide (29.0 mg, 0.060 mmol, 29%) as a yellow solid. ¹H NMR (500 MHz, DMSO-d₆) δ 11.04 (s, 1H), 9.07-8.97 (m, 2H), 8.87 (t, J=3.5 Hz, 2H), 8.32 (dd, J=6.2, 2.2 Hz, 2H), 8.25 (s, 1H), 7.78 (d, J=4.9 Hz, 1H), 6.96 (d, J=5.0 Hz, 1H), 6.64 (s, 1H), 5.46 (q, J=6.9 Hz, 1H), 3.45 (s, 3H), 2.88 (d, J=4.9 Hz, 3H), 2.50 (dt, J=3.4, 1.7 Hz, 3H). MS (ESI) m/z 483.0 [M+H]⁺;
and the second eluting enantiomer (retention time 2.4 min) which was arbitrarily assigned as (S)—N-(6-methyl-5-(7-(methylamino)-1,6-naphthyridin-3-yl)pyridin-3-yl)-4-(2,2,2-trifluoro-1-methoxyethyl)picolinamide (40.0 mg, 0.083 mmol, 40%) as a yellow solid. ¹H NMR (500 MHz, DMSO-d₆) δ 11.04 (s, 1H), 9.07-8.97 (m, 2H), 8.87 (t, J=3.5 Hz, 2H), 8.32 (dd, J=6.2, 2.2 Hz, 2H), 8.25 (s, 1H), 7.78 (d, J=4.9 Hz, 1H), 6.96 (d, J=5.0 Hz, 1H), 6.64 (s, 1H), 5.46 (q, J=6.9 Hz, 1H), 3.45 (s, 3H), 2.88 (d, J=4.9 Hz, 3H), 2.50 (dt, J=3.4, 1.7 Hz, 3H). MS (ESI) m/z 483.0 [M+H]⁺

### Example 127. Synthesis of 3-(difluoromethyl)-N-(6-methyl-5-(7-(methylamino)-1,6-naphthyridin-3-yl)pyridin-3-yl)benzamide (Compound 143)

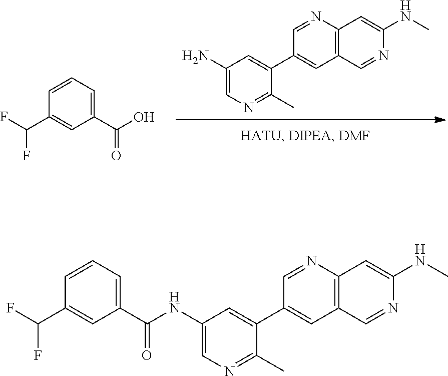 [SMILES: O=C(O)c1cccc(C(F)F)c1 ; CNc1cc2ncc(-c3cc(N)cnc3C)cc2cn1 ; CNc1cc2ncc(-c3cc(NC(=O)c4cccc(C(F)F)c4)cnc3C)cc2cn1]

A solution of 3-(difluoromethyl)benzoic acid (40 mg, 0.23 mmol), 3-(5-amino-2-methylpyridin-3-yl)-N-methyl-1,6-naphthyridin-7-amine (62 mg, 0.23 mmol), 1-[bis(dimethylamino)methylene]-1H-1,2,3-triazolo[4,5-b]pyridinium 3-oxid hexafluorophosphate (175 mg, 0.46 mmol) and N,N-diisopropylethylamine (0.2 mL) in N,N-dimethylformamide (1 mL) was stirred at room temperature for 1 hour. The solution was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-(difluoromethyl)-N-(6-methyl-5-(7-(methylamino)-1,6-naphthyridin-3-yl)pyridin-3-yl)benzamide (57.4 mg, 0.14 mmol, 59%) as a yellow solid. ¹H NMR (400 MHz, DMSO-d₆) δ 10.66 (s, 1H), 8.99 (s, 1H), 8.90 (d, J=2.4 Hz, 1H), 8.66 (d, J=2.8 Hz, 1H), 8.32 (d, J=1.6 Hz, 1H), 8.20-8.16 (m, 3H), 7.81 (d, J=8.0 Hz, 1H), 7.72 (t, J=7.6 Hz, 1H), 7.16 (t, J=56.0 Hz, 1H), 6.97-6.96 (m, 1H), 6.63 (s, 1H), 2.87 (d, J=4.8 Hz, 3H), 2.49 (s, 3H). MS (ESI) m/z 420.0 [M+H]⁺

### Example 128. Synthesis of 3-(difluoromethyl)-N-(4-methyl-3-(7-(methylamino)-1,6-naphthyridin-3-yl)phenyl)benzamide (Compound 144)

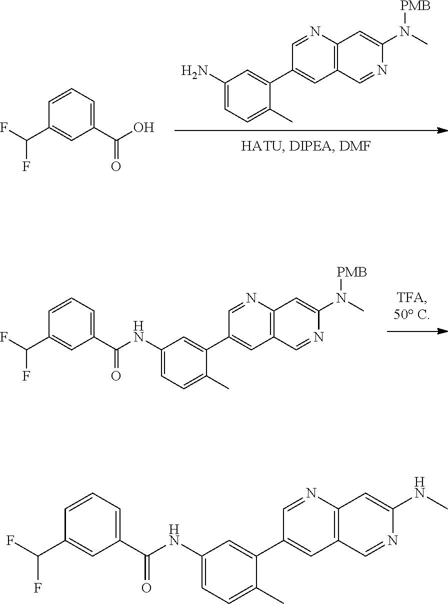

Step 1. A solution of 3-(difluoromethyl)benzoic acid (40 mg, 0.23 mmol), 3-(5-amino-2-methylphenyl)-N-(4-methoxybenzyl)-N-methyl-1,6-naphthyridin-7-amine (88 mg, 0.23 mmol), N,N-diisopropylethylamine (89 mg, 0.69 mmol) and 1-[bis(dimethylamino)methylene]-1H-1,2,3-triazolo[4,5-b]pyridinium 3-oxid hexafluorophosphate (175 mg, 0.46 mmol) in N,N-dimethylformamide (1 mL) was stirred at room temperature for 1 hour. The reaction was poured into water (20 mL) and extracted with ethyl acetate (20 mL×3). The combined organic phases were concentrated and purified by flash chromatography (silica, petroleum ether/ethyl acetate=1/2) to give 3-(difluoromethyl)-N-(3-(7-((4-methoxybenzyl)(methyl)amino)-1,6-naphthyridin-3-yl)-4-methylphenyl)benzamide (80 mg, 0.15 mmol, 64%) as a yellow solid. MS (ESI) m/z 539.1 [M+H]⁺

Step 2. A solution of 3-(difluoromethyl)-N-(3-(7-((4-methoxybenzyl)(methyl)amino)-1,6-naphthyridin-3-yl)-4-methylphenyl)benzamide (80 mg, 0.15 mmol) in trifluoroacetic acid (4 mL) was stirred at 50° C. overnight. After cooling to room temperature, the reaction mixture was adjusted to pH=8 with saturated aqueous sodium bicarbonate solution. The mixture was concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-(difluoromethyl)-N-(4-methyl-3-(7-(methylamino)-1,6-naphthyridin-3-yl)phenyl)benzamide (51.7 mg, 0.12 mmol, 82%) as a yellow solid. ¹H NMR (400 MHz, DMSO-d₆) δ 10.45 (s, 1H), 8.99 (s, 1H), 8.82 (d, J=2.4 Hz, 1H), 8.25 (d, J=2.0 Hz, 1H), 8.17-8.13 (m, 2H), 7.80-7.77 (m, 3H), 7.69 (t, J=8.0 Hz, 1H), 7.36-7.34 (m, 1H), 7.15 (t, J=55.6 Hz, 1H), 6.93-6.91 (m, 1H), 6.62 (s, 1H), 2.86 (d, J=4.8 Hz, 3H), 2.29 (s, 3H). MS (ESI) m/z 419.1 [M+H]⁺

### Example 129. Synthesis of N-(6-methyl-5-(7-(methylamino)-1,6-naphthyridin-3-yl)pyridin-3-yl)-3-(trifluoromethyl)benzamide (Compound 145)

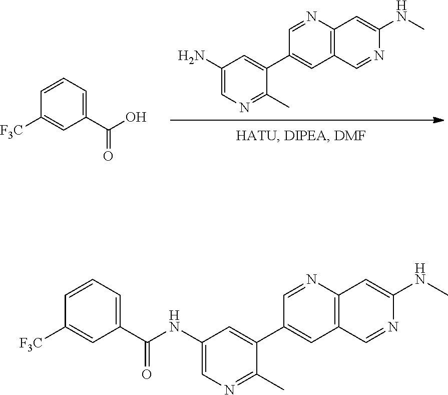 [SMILES: O=C(O)c1cccc(C(F)(F)F)c1 ; CNc1cc2ncc(-c3cc(N)cnc3C)cc2cn1 ; CNc1cc2ncc(-c3cc(NC(=O)c4cccc(C(F)(F)F)c4)cnc3C)cc2cn1]

A solution of 3-(trifluoromethyl)benzoic acid (50 mg, 0.26 mmol), 3-(5-amino-2-methylpyridin-3-yl)-N-methyl-1,6-naphthyridin-7-amine (48.8 mg, 0.18 mmol), 1-[bis(dimethylamino)methylene]-1H-1,2,3-triazolo[4,5-b]pyridinium 3-oxid hexafluorophosphate (150 mg, 0.39 mmol) and N,N-diisopropylethylamine (101.8 mg, 0.79 mmol) in N,N-dimethylformamide (3 mL) was stirred at room temperature for 2 hours. The residue was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-3-(trifluoromethyl)benzamide (36.8 mg, 0.084 mmol, 32%) as a yellow solid. ¹H NMR (400 MHz, DMSO-d₆) δ 10.72 (s, 1H), 9.01 (s, 1H), 8.89 (dd, J=11.4, 2.4 Hz, 2H), 8.34 (s, 2H), 8.30 (d, J=7.8 Hz, 1H), 8.18 (d, J=2.3 Hz, 1H), 8.00 (d, J=7.7 Hz, 1H), 7.82 (t, J=7.8 Hz, 1H), 6.99 (s, 1H), 6.64 (s, 1H), 2.88 (d, J=3.7 Hz, 3H), 2.49 (s, 3H). MS (ESI) m/z 438.1 [M+H]⁺

### Example 130. Synthesis of 5-methyl-N-(4-methyl-3-(7-(methylamino)-1,6-naphthyridin-3-yl)phenyl)-4,5,6,7-tetrahydrobenzo[d]isoxazole-3-carboxamide (Compound 146)

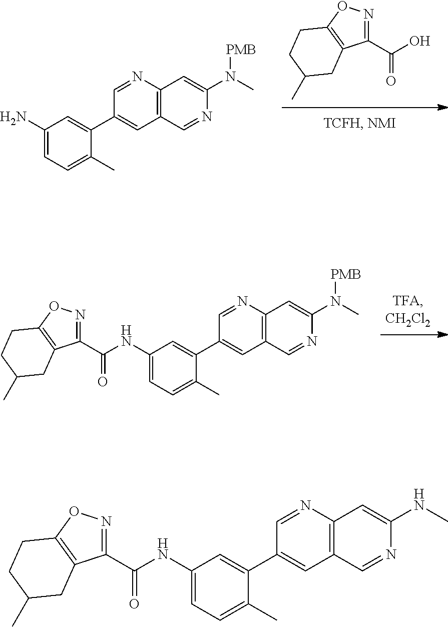

Step 1. To a solution of 3-(5-amino-2-methyl-phenyl)-N-[(4-methoxyphenyl)methyl]-N-methyl-1,6-naphthyridin-7-amine (0.1 g, 0.26 mmol) in acetonitrile (0.3 mL) were added 5-methyl-4,5,6,7-tetrahydro-1,2-benzoxazole-3-carboxylic acid (47 mg, 0.26 mmol), chloro-N,N,N′,N′-tetramethylformamidinium hexafluorophosphate (TCFH, 0.15 g, 0.52 μmol) and 1-methylimidazole (85 mg, 1 mmol). The mixture was stirred at 25° C. for 12 hours. The mixture was concentrated in vacuo to give a residue. The residue was purified by reversed-phase HPLC (0.1% formic acid) to give N-(3-(7-((4-methoxybenzyl)(methyl)amino)-1,6-naphthyridin-3-yl)-4-methylphenyl)-5-methyl-4,5,6,7-tetrahydrobenzo[d]isoxazole-3-carboxamide (50 mg, 35%) as a yellow oil. MS (ESI) m/z 548.3 [M+H]⁺

Step 2. To a solution of N-[3-[7-[(4-methoxyphenyl)methyl-methyl-amino]-1,6-naphthyridin-3-yl]-4-methyl-phenyl]-5-methyl-4,5,6,7-tetrahydro-1,2-benzoxazole-3-carboxamide (50 mg, 91 μmol) in dichloromethane (1 mL) was added trifluoroacetic acid (0.46 g, 4.1 mmol). The mixture was stirred at 25° C. for 2 hours. The mixture was concentrated in vacuo to give a residue. The residue was purified by prep-HPLC (Column: Phenomenex Synergi 25×150 mm C18, 10 μm, Mobile Phase: A: water (formic acid), B: acetonitrile; B %: 32%-62% in 10 min) to give 5-methyl-N-(4-methyl-3-(7-(methylamino)-1,6-naphthyridin-3-yl)phenyl)-4,5,6,7-tetrahydrobenzo[d]isoxazole-3-carboxamide (24 mg, 61%) as a yellow solid. ¹H NMR (400 MHz, DMSO-d₆) δ 10.65 (s, 1H), 8.98 (s, 1H), 8.79 (d, J=2.4 Hz, 1H), 8.22 (d, J=2.0 Hz, 1H), 7.81 (d, J=2.0 Hz, 1H), 7.72 (dd, J=2.4, 8.4 Hz, 1H), 7.33 (d, J=8.4 Hz, 1H), 6.89 (q, J=4.8 Hz, 1H), 6.62 (s, 1H), 2.87 (d, J=5.2 Hz, 3H), 2.83-2.65 (m, 3H), 2.27 (s, 3H), 2.14 (dd, J=9.2, 15.6 Hz, 1H), 1.98-1.76 (m, 2H), 1.47 (dtd, J=6.0, 10.4, 13.2 Hz, 1H), 1.04 (d, J=6.4 Hz, 3H). MS (ESI) m/z 428.0 [M+H]⁺

### Example 131. Synthesis of 5-cyclopropyl-1-ethyl-N-(4-methyl-3-(7-(methylamino)-1,6-naphthyridin-3-yl)phenyl)-1H-pyrazole-3-carboxamide (Compound 147)

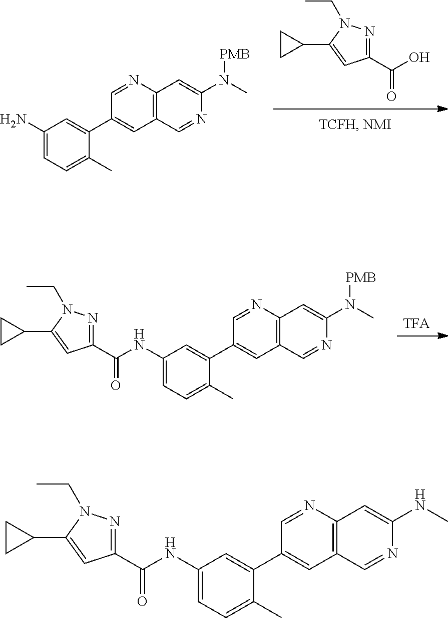

Step 1. To a solution of 3-(5-amino-2-methyl-phenyl)-N-[(4-methoxyphenyl)methyl]-N-methyl-1,6-naphthyridin-7-amine (0.1 g, 0.26 mmol) in acetonitrile (0.3 mL) was added 5-cyclopropyl-1-ethyl-pyrazole-3-carboxylic acid (47 mg, 0.26 mmol), chloro-N,N,N′,N′-tetramethylformamidinium hexafluorophosphate (0.15 g, 0.52 mmol) and 1-methylimidazole (85 mg, 1 mmol). The mixture was stirred at 25° C. for 12 hours. The mixture was concentrated in vacuo to give a residue. The residue was purified by reversed-phase HPLC (0.1% formic acid) to give 5-cyclopropyl-1-ethyl-N-(3-(7-((4-methoxybenzyl)(methyl)amino)-1,6-naphthyridin-3-yl)-4-methylphenyl)-1H-pyrazole-3-carboxamide (0.1 g, 70%) as a yellow oil. MS (ESI) m/z 547.4 [M+H]⁺

Step 2. To a solution of 5-cyclopropyl-1-ethyl-N-[3-[7-[(4-methoxyphenyl)methyl-methyl-amino]-1,6-naphthyridin-3-yl]-4-methyl-phenyl]pyrazole-3-carboxamide (0.1 g, 0.18 mmol) in dichloromethane (1.5 mL) was added trifluoroacetic acid (0.46 g, 4.1 mmol). The mixture was stirred at 25° C. for 1 hour. The mixture was concentrated in vacuo to give a residue. The residue was purified by prep-HPLC (Column: Phenomenex Synergi 25×150 mm C18, 10 μm, Mobile Phase: A: water (formic acid), B: acetonitrile; B %: 24%-54% in 10 min) to give 5-cyclopropyl-1-ethyl-N-(4-methyl-3-(7-(methylamino)-1,6-naphthyridin-3-yl)phenyl)-1H-pyrazole-3-carboxamide (47 mg, 60%) as a yellow solid. ¹H NMR (400 MHz, DMSO-d&) δ 9.86 (s, 1H), 8.97 (s, 1H), 8.80 (d, J=2.4 Hz, 1H), 8.21 (d, J=1.6 Hz, 1H), 7.83-7.75 (m, 2H), 7.29 (d, J=8.0 Hz, 1H), 6.87 (q, J=4.8 Hz, 1H), 6.62 (s, 1H), 6.39 (s, 1H), 4.29 (q, J=7.2 Hz, 2H), 2.87 (d, J=5.2 Hz, 3H), 2.26 (s, 3H), 2.01-1.90 (m, 1H), 1.42 (t, J=7.2 Hz, 3H), 1.02-0.94 (m, 2H), 0.75-0.66 (m, 2H). MS (ESI) m/z 427.0 [M+H]⁺

### Example 132. Synthesis of N-(6-methyl-5-(7-(methylamino)-1,6-naphthyridin-3-yl)pyridin-3-yl)-5-(trifluoromethyl)oxazol-2-amine (Compound 148)

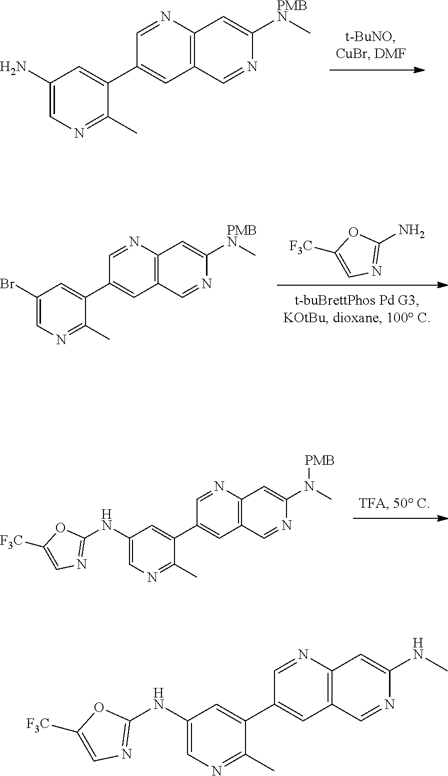

Step 1. To a solution of tert-butyl nitrite (107 mg, 1.04 mmol) and copper (I) bromide (111 mg, 0.78 mmol) in N,N-dimethylformamide (4 mL) was added 3-(5-amino-2-methylpyridin-3-yl)-N-(4-methoxybenzyl)-N-methyl-1,6-naphthyridin-7-amine (200 mg, 0.52 mmol) at 20° C. The reaction mixture was stirred at 20° C. for 16 hours. The reaction was diluted with ethyl acetate (50 mL×3), washed with saturated sodium bicarbonate aqueous solution (50 mL) and brine (40 mL), dried over sodium sulfate, filtered, and concentrated. The residue was purified by flash chromatography (silica, ethyl acetate) to give 3-(5-bromo-2-methylpyridin-3-yl)-N-(4-methoxybenzyl)-N-methyl-1,6-naphthyridin-7-amine (100 mg, 0.22 mmol, 43%) as a yellow solid. MS (EST) m/z 449.1 [M+H]⁺

Step 2. A mixture of 5-(trifluoromethyl)oxazol-2-amine (20 mg, 0.132 mmol), 3-(5-bromo-2-methylpyridin-3-yl)-N-(4-methoxybenzyl)-N-methyl-1,6-naphthyridin-7-amine (30 mg, 0.067 mmol), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (6 mg, 0.007 mmol) and potassium tert-butoxide (23 mg, 0.2 mmol) in 1,4-dioxane (2 mL) was stirred at 100° C. for 16 hours under nitrogen atmosphere. After cooling to room temperature, the reaction was diluted with water (20 mL) and extracted with ethyl acetate (20 mL×2). The combined organic phases were washed with brine (30 mL), dried over sodium sulfate, filtered, and concentrated. The crude residue was purified by flash chromatography (silica, methanol/dichloromethane=1/10) to give N-(5-(7-((4-methoxybenzyl)(methyl)amino)-1,6-naphthyridin-3-yl)-6-methylpyridin-3-yl)-5-(trifluoromethyl)oxazol-2-amine (20 mg, 0.038 mmol, 57%) as a yellow solid. MS (ESI) m/z 521.1 [M+H]⁺

Step 3. A solution of N-(5-(7-((4-methoxybenzyl)(methyl)amino)-1,6-naphthyridin-3-yl)-6-methylpyridin-3-yl)-5-(trifluoromethyl)oxazol-2-amine (20 mg, 0.038 mmol) in trifluoroacetic acid (10 mL) was stirred at 50° C. for 1 hour. The mixture was concentrated and purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(6-methyl-5-(7-(methylamino)-1,6-naphthyridin-3-yl)pyridin-3-yl)-5-(trifluoromethyl)oxazol-2-amine (3.8 mg, 0.0095 mmol, 25%) as a yellow solid. ¹H NMR (400 MHz, DMSO-d₆) δ 10.84 (s, 1H), 9.00 (s, 1H), 8.84 (d, J=2.3 Hz, 1H), 8.71 (d, J=2.5 Hz, 1H), 8.44 (d, J=1.8 Hz, 1H), 8.33 (d, J=1.8 Hz, 1H), 7.94 (d, J=2.5 Hz, 1H), 7.00 (s, 1H), 6.63 (s, 1H), 2.88 (s, 3H), 2.45 (s, 3H). MS (ESI) m/z 401.1 [M+H]⁺

### Example 133. Synthesis of 3-(2-cyanopropan-2-yl)-N-(6-methyl-5-(7-(methylamino)-1,6-naphthyridin-3-yl)pyridin-3-yl)benzamide (Compound 149)

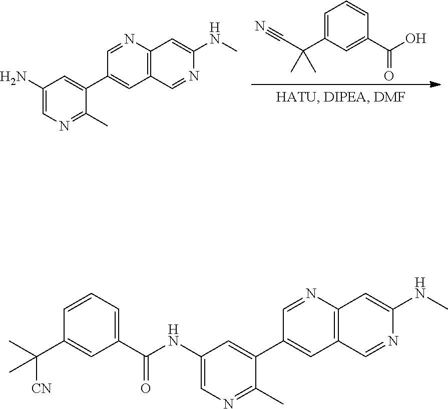

A solution of 3-(2-cyanopropan-2-yl)benzoic acid (30 mg, 0.16 mmol), 3-(5-amino-2-methylpyridin-3-yl)-N-methyl-1,6-naphthyridin-7-amine (33.7 mg, 0.13 mmol), 1-[bis(dimethylamino)methylene]-1H-1,2,3-triazolo[4,5-b]pyridinium 3-oxid hexafluorophosphate (91.2 mg, 0.24 mmol) and N,N-diisopropylethylamine (61.9 mg, 0.48 mmol) in N,N-dimethylformamide (3 mL) was stirred at room temperature for 2 hours. The reaction solution was purified by prep-HPLC (Column: Xbridge 21.2×250 mm C18, 10 μm, Mobile Phase A: water (10 mM ammonium bicarbonate), B: acetonitrile) to give 3-(2-cyanopropan-2-yl)-N-(6-methyl-5-(7-(methylamino)-1,6-naphthyridin-3-yl)pyridin-3-yl)benzamide (38 mg, 0.087 mmol, 55%) as a yellow solid. ¹H NMR (400 MHz, DMSO-d₆) δ 10.56 (s, 1H), 9.00 (s, 1H), 8.88 (dd, J=10.7, 2.3 Hz, 2H), 8.32 (d, J=2.1 Hz, 1H), 8.17 (d, J=2.3 Hz, 1H), 8.09 (s, 1H), 7.99 (d, J=7.8 Hz, 1H), 7.78 (d, J=8.1 Hz, 1H), 7.63 (t, J=7.8 Hz, 1H), 6.95 (d, J=5.0 Hz, 1H), 6.64 (s, 1H), 2.88 (d, J=4.9 Hz, 3H), 2.51 (s, 3H), 1.76 (s, 6H). MS (ESI) m/z 437.1 [M+H]⁺

### Example 134. Synthesis of N-methyl-3-(2-methyl-5-((1-methyl-2-(trifluoromethyl)-1H-imidazol-4-yl)amino)pyridin-3-yl)-1,6-naphthyridin-7-amine (Compound 150)

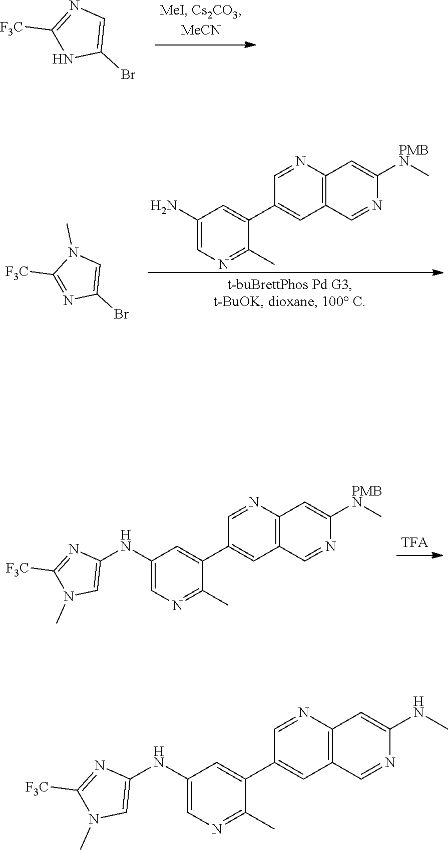

Step 1. To a solution of 5-bromo-2-(trifluoromethyl)-1H-imidazole (500 mg, 2.32 mmol) and cesium carbonate (1.51 g, 4.64 mmol) in acetonitrile (15 mL) was added dropwise methyl iodide (494 mg, 3.48 mmol) and the resulting solution was stirred for 12 hours at 20° C. The reaction mixture was quenched with water (50 mL) and extracted with ethyl acetate (3×80 mL). The combined organic layers were washed with brine (2×100 mL) and dried over anhydrous sodium sulfate. After filtration, the filtrate was concentrated reduced pressure. The residue was purified by flash chromatography (silica, ethyl acetate/petroleum ether=1/4) to afford 4-bromo-1-methyl-2-(trifluoromethyl)-1H-imidazole (250 mg, 1.09 mmol, 47%) as a yellow oil. MS (ESI) m/z 229.1 [M+H]⁺

Step 2. To a solution of 4-bromo-1-methyl-2-(trifluoromethyl)-1H-imidazole (30 mg, 0.13 mmol) and 3-(5-amino-2-methylpyridin-3-yl)-N-(4-methoxybenzyl)-N-methyl-1,6-naphthyridin-7-amine (50 mg, 0.13 mmol) in 1,4-dioxane (5 mL) were added [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (10 mg, 0.012 mmol) and potassium tert-butoxide (29 mg, 0.26 mmol). The mixture was stirred for 16 hours at 100° C. under nitrogen atmosphere. The mixture was concentrated reduced pressure. The residue was purified by flash chromatography (silica, ethyl acetate/petroleum ether=1/4) to afford N-(4-methoxybenzyl)-N-methyl-3-(2-methyl-5-((1-methyl-2-(trifluoromethyl)-1H-imidazol-4-yl)amino)pyridin-3-yl)-1,6-naphthyridin-7-amine (20 mg, 0.037 mmol, 29%) as a yellow solid. MS (ESI) m/z 534.2 [M+H]⁺

Step 3. A mixture of N-(4-methoxybenzyl)-N-methyl-3-(2-methyl-5-((1-methyl-2-(trifluoromethyl)-1H-imidazol-4-yl)amino)pyridin-3-yl)-1,6-naphthyridin-7-amine (20 mg, 0.038 mmol) in trifluoroacetic acid (4 mL) was stirred for 12 hours at 20° C. and concentrated under reduced pressure. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-methyl-3-(2-methyl-5-((1-methyl-2-(trifluoromethyl)-1H-imidazol-4-yl)amino)pyridin-3-yl)-1,6-naphthyridin-7-amine (6.2 mg, 0.015 mmol, 39%) as a yellow solid. ¹H NMR (400 MHz, DMSO-d₆) δ 8.97 (s, 1H), 8.81 (d, J=2.4 Hz, 1H), 8.64 (s, 1H), 8.31 (d, J=3.2 Hz, 1H), 8.26 (d, J=1.2 Hz, 1H), 7.37 (d, J=2.8 Hz, 1H), 7.23 (s, 1H), 6.93 (d, J=5.2 Hz, 1H), 6.62 (s, 1H), 3.76 (d, J=0.8 Hz, 3H), 2.87 (d, J=4.8 Hz, 3H), 2.36 (s, 3H). MS (ESI) m/z 414.1 [M+H]⁺

### Example 135. Synthesis of 3-(1-cyanocyclopropyl)-N-(4-methyl-3-(7-(methylamino)-1,6-naphthyridin-3-yl)phenyl)benzamide (Compound 151)

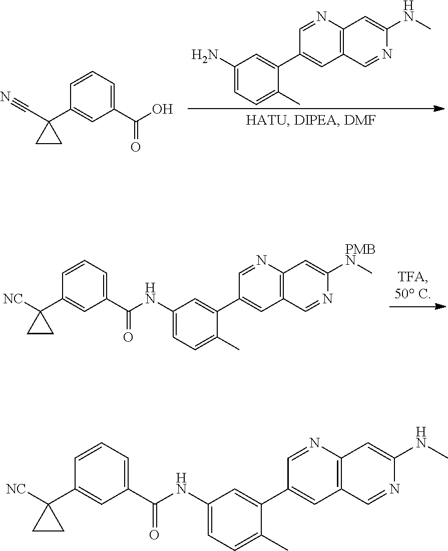

Step 1. To a solution of 3-(1-cyanocyclopropyl)benzoic acid (30 mg, 0.16 mmol) in N,N-dimethylformamide (5 mL) were added 1-[bis(dimethylamino)methylene]-1H-1,2,3-triazolo[4,5-b]pyridinium 3-oxid hexafluorophosphate (73 mg, 0.19 mmol), N,N-diisopropylethylamine (60 mg, 0.48 mmol) and 3-(5-amino-2-methylphenyl)-N-(4-methoxybenzyl)-N-methyl-1,6-naphthyridin-7-amine (42 mg, 0.16 mmol). The reaction mixture was stirred at room temperature for 1 hour. The reaction mixture was diluted with ethyl acetate (100 mL), washed with water (50 mL×2) and brine (50 mL), dried over sodium sulfate, filtered, and concentrated. The crude product was purified by flash chromatography (silica, petroleum ether/ethyl acetate=1/4) to 3-(1-cyanocyclopropyl)-N-(3-(7-((4-methoxybenzyl)(methyl)amino)-1,6-naphthyridin-3-yl)-4-methylphenyl)benzamide (50 mg, 0.09 mmol, 56%) as a yellow oil. MS (EST) m/z 554 [M+H]⁺

Step 2. A mixture of 3-(1-cyanocyclopropyl)-N-(3-(7-((4-methoxybenzyl)(methyl)amino)-1,6-naphthyridin-3-yl)-4-methylphenyl)benzamide (50 mg, 0.09 mmol) in trifluoroacetic acid (5 mL) was stirred at 50° C. for 1 hour. After cooling to room temperature, the reaction mixture was concentrated. The residue was purified by prep-HPLC (Column: Xbridge 21.2×250 mm C18, 10 μm, Mobile Phase A: water (10 mM ammonium bicarbonate), B: acetonitrile) to give 3-(1-cyanocyclopropyl)-N-(4-methyl-3-(7-(methylamino)-1,6-naphthyridin-3-yl)phenyl)benzamide (26.2 mg, 0.061 mmol, 68%) as a yellow solid. ¹H NMR (400 MHz, DMSO-d₆) δ 10.34 (s, 1H), 8.99 (s, 1H), 8.82 (d, J=2.3 Hz, 1H), 8.24 (d, J=1.7 Hz, 1H), 7.96-7.81 (m, 2H), 7.79-7.68 (m, 2H), 7.56 (dd, J=4.8, 1.4 Hz, 2H), 7.35 (d, J=9.1 Hz, 1H), 6.90 (d, J=5.1 Hz, 1H), 6.62 (s, 1H), 2.87 (d, J=5.0 Hz, 3H), 2.31 (d, J=17.1 Hz, 3H), 1.82 (q, J=4.8 Hz, 2H), 1.62 (q, J=5.1 Hz, 2H). MS (ESI) m/z 434 [M+H]⁺

### Example 136. Synthesis of N-(3-(7-(ethylamino)-1,6-naphthyridin-3-yl)-4-methylphenyl)-2-(1-fluorocyclopropyl)isonicotinamide (Compound 153)

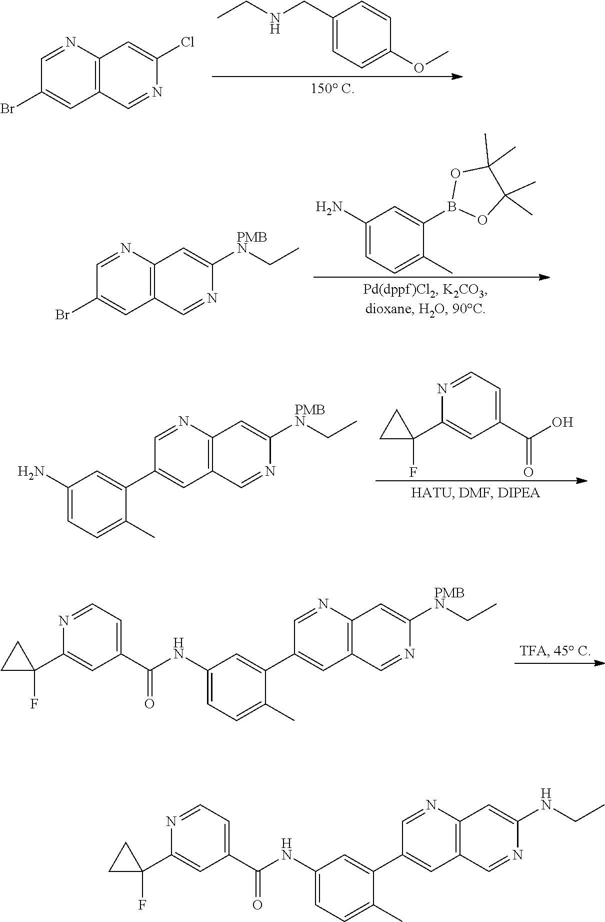

Step 1. A solution of 3-bromo-7-chloro-1,6-naphthyridine (330 mg, 1.36 mmol) in N-(4-methoxybenzyl)ethanamine (675 g, 4.09 mmol) was stirred at 150° C. for 16 hours. The reaction was quenched with water (100 mL) and extracted with ethyl acetate (100 mL×3). The combined organic layers were washed with brine (100 mL), dried over anhydrous sodium sulfate, filtered, and concentrated. The residue was purified by flash chromatography (silica, petroleum ether/ethyl acetate=5/2) to give 3-bromo-N-ethyl-N-(4-methoxybenzyl)-1,6-naphthyridin-7-amine (240 mg, 0.65 mmol, 48%) as yellow solid. MS (ESI) m/z 372.0 [M+H]⁺

Step 2. A solution of 3-bromo-N-ethyl-N-(4-methoxybenzyl)-1,6-naphthyridin-7-amine (100 mg, 0.27 mmol), 4-methyl-3-(4,4,5,5-tetramethyl-1,3,2-dioxaborolan-2-yl)aniline (75 mg, 0.32 mmol), 1,1′-bis(diphenylphosphino)ferrocene-palladium(II)dichloride (22 mg, 0.03 mmol) and potassium carbonate (112 mg, 0.81 mmol) in water (1 mL) and dioxane (5 mL) was stirred at 90° C. for 16 hours under argon. After cooling to room temperature, the mixture was diluted with ethyl acetate (50 mL) and washed with water (50 mL). The organic layer was concentrated and purified by flash chromatography (silica, petroleum ether/ethyl acetate=1/1) give 3-(5-amino-2-methylphenyl)-N-ethyl-N-(4-methoxybenzyl)-1,6-naphthyridin-7-amine (40 mg, 0.10 mmol, 35%) as a yellow solid. MS (ESI) m/z 399.1 [M+H]⁺

Step 3. To a solution of 3-(5-amino-2-methylphenyl)-N-ethyl-N-(4-methoxybenzyl)-1,6-naphthyridin-7-amine (40 mg, 0.10 mmol) in N,N-dimethylformamide (3 mL) was added N,N-diisopropylethylamine (39 mg, 0.30 mmol), 1-[bis(dimethylamino)methylene]-1H-1,2,3-triazolo[4,5-b]pyridinium 3-oxid hexafluorophosphate (57 mg, 0.15 mmol) and 2-(1-fluorocyclopropyl)isonicotinic acid (18 mg, 0.10 mmol) at room temperature. The mixture was stirred at room temperature for 2 hours. The reaction was diluted with ethyl acetate (100 mL), washed with saturated sodium bicarbonate aqueous solution (50 mL) and brine (40 mL), dried over sodium sulfate, filtered, and concentrated. The residue was purified by flash chromatography (silica, petroleum ether/ethyl acetate=1/1) to give N-(3-(7-(ethyl(4-methoxybenzyl)amino)-1,6-naphthyridin-3-yl)-4-methylphenyl)-2-(1-fluorocyclopropyl)isonicotinamide (60 mg, 0.11 mmol, 89%) as a yellow solid. MS (ESI) m/z 562.2 [M+H]⁺

Step 4. A mixture of N-(3-(7-(ethyl(4-methoxybenzyl)amino)-1,6-naphthyridin-3-yl)-4-methylphenyl)-2-(1-fluorocyclopropyl)isonicotinamide (60 mg, 0.107 mmol) in trifluoroacetic acid (4 mL) was stirred at 45° C. for 2 hours. The reaction was concentrated and the residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(3-(7-(ethylamino)-1,6-naphthyridin-3-yl)-4-methylphenyl)-2-(1-fluorocyclopropyl)isonicotinamide (13.1 mg, 0.030 mmol, 28%) as a yellow solid. ¹H NMR (400 MHz, DMSO-d₆) δ 1.22 (t, J=7.2 Hz, 3H), 1.37-1.43 (m, 2H), 1.54-1.62 (m, 2H), 2.29 (s, 3H), 3.54 (s, 2H), 6.67 (s, 1H), 7.00 (s, 1H), 7.37 (d, J=8.0 Hz, 1H), 7.76-7.78 (m, 3H), 8.08 (s, 1H), 8.29 (s, 1H), 8.70 (d, J=7.2 Hz, 1H), 8.83 (d, J=2.4 Hz, 1H), 9.01 (s, 1H), 10.63 (s, 1H). MS (ESI) m/z 442.2 [M+H]⁺

### Example 137. Synthesis of N-(3-(7-amino-1,6-naphthyridin-3-yl)-4-methylphenyl)-2-(1-fluorocyclopropyl)isonicotinamide (Compound 154)

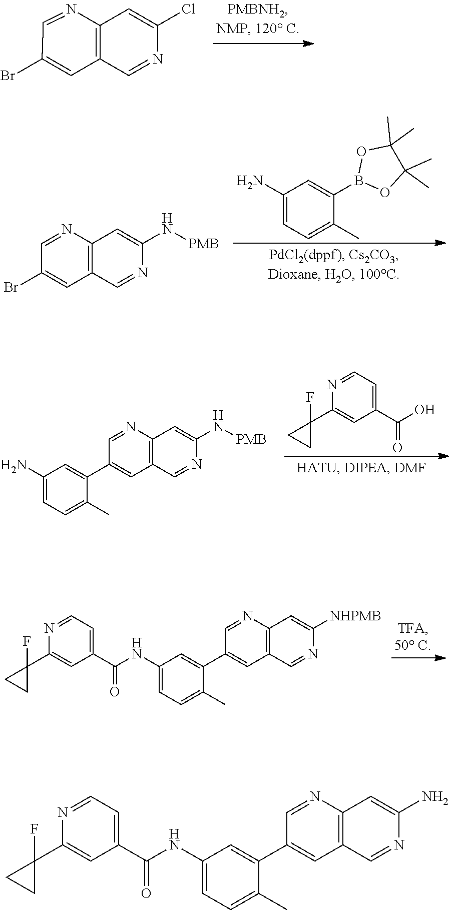

Step 1. A solution of 3-bromo-7-chloro-1,6-naphthyridine (0.33 g, 1.37 mmol) and (4-methoxyphenyl)methanamine (0.56 g, 4.12 mmol) in 1-methyl-2-pyrrolidinone (2 mL) was stirred at 120° C. for 16 hours. The mixture was poured into water (20 mL) and filtered. The filter cake was washed with ethyl acetate (40 mL) and dried to give 3-bromo-N-(4-methoxybenzyl)-1,6-naphthyridin-7-amine (120 mg, 0.35 mmol, 27%) as a yellow solid. MS (ESI) m/z 344.0 [M+H]⁺

Step 2. A mixture of 3-bromo-N-(4-methoxybenzyl)-1,6-naphthyridin-7-amine (180 mg, 0.523 mmol), 1,1′-bis(diphenylphosphino)ferrocene-palladium(II)dichloride dichloromethane complex (43 mg, 0.053 mmol), cesium carbonate (511 mg, 1.57 mmol) and 4-methyl-3-(4,4,5,5-tetramethyl-1,3,2-dioxaborolan-2-yl)aniline (158 mg, 0.68 mmol) in dioxane (5 mL) and water (2 mL) was stirred at 100° C. for 3 hours under nitrogen. After cooling to room temperature, the reaction mixture was poured into water (10 mL) and extracted with ethyl acetate (20 mL×3). The combined organic phases were concentrated and purified by flash chromatography (silica, petroleum methanol/dichloromethane=1/10) to give 3-(5-amino-2-methylphenyl)-N-(4-methoxybenzyl)-1,6-naphthyridin-7-amine (160 mg, 0.43 mmol, 82%) as a yellow solid. MS (ESI) m/z 371.1 [M+H]⁺

Step 3. A mixture of 3-(5-amino-2-methylphenyl)-N-(4-methoxybenzyl)-1,6-naphthyridin-7-amine (160 mg, 0.43 mmol), 2-(1-fluorocyclopropyl)isonicotinic acid (78 mg, 0.43 mmol), 1-[bis(dimethylamino)methylene]-1H-1,2,3-triazolo[4,5-b]pyridinium 3-oxid hexafluorophosphate (490 mg, 1.29 mmol) and N,N-diisopropylethylamine (166 mg, 1.29 mmol) in N,N-dimethylformamide (10 mL) was stirred at room temperature for 1 hour. The mixture was poured into water (20 mL) and extracted with dichloromethane (20 mL×3). The organic phases were concentrated and purified by flash chromatography (silica, methanol/dichloromethane=1/20) to give 2-(1-fluorocyclopropyl)-N-(3-(7-(4-methoxybenzylamino)-1,6-naphthyridin-3-yl)-4-methylphenyl)isonicotinamide (160 mg, 0.3 mmol, 70%) as a yellow solid. MS (ESI) m/z 534.0 [M+H]⁺

Step 4. A solution of 2-(1-fluorocyclopropyl)-N-(3-(7-(4-methoxybenzylamino)-1,6-naphthyridin-3-yl)-4-methylphenyl)isonicotinamide (160 mg, 0.3 mmol) in trifluoroacetic acid (6 mL) was stirred at 50° C. for 1 hour. The reaction mixture was cooled to room temperature and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(3-(7-amino-1,6-naphthyridin-3-yl)-4-methylphenyl)-2-(1-fluorocyclopropyl)isonicotinamide (54.4 mg, 0.13 mmol, 44%) as a yellow solid. ¹H NMR (400 MHz, DMSO-d₆) δ 10.62 (s, 1H), 8.95 (s, 1H), 8.81 (d, J=2.3 Hz, 1H), 8.71 (d, J=5.1 Hz, 1H), 8.24 (d, J=1.9 Hz, 1H), 8.08 (s, 1H), 7.77 (dd, J=3.4, 1.8 Hz, 3H), 7.46-7.25 (m, 1H), 6.77 (s, 1H), 6.42 (s, 2H), 2.29 (s, 3H), 1.59 (ddd, J=19.0, 8.1, 5.1 Hz, 2H), 1.40 (td, J=8.3, 5.3 Hz, 2H). MS (ESI) m/z 414.1 [M+H]⁺

### Example 138. Synthesis of 4-(2-cyanopropan-2-yl)-N-(3-(7-(ethylamino)-1,6-naphthyridin-3-yl)-4-methylphenyl)picolinamide (Compound 173)

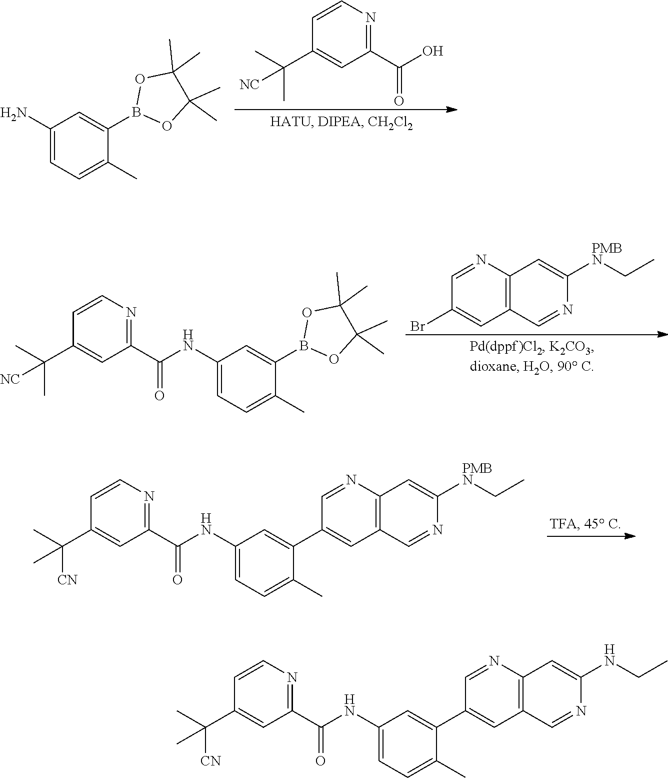

Step 1. A mixture of 4-methyl-3-(4,4,5,5-tetramethyl-1,3,2-dioxaborolan-2-yl)aniline (480 mg, 2.06 mmol), 4-(2-cyanopropan-2-yl)picolinic acid (391 mg, 2.06 mmol), 1-[bis(dimethylamino)methylene]-1H-1,2,3-triazolo[4,5-b]pyridinium 3-oxid hexafluorophosphate (1.5 g, 4.12 mmol) and N,N-diisopropylethylamine (0.79 g, 6.18 mmol) in dichloromethane (15 mL) was stirred at room temperature for 0.5 hour. The mixture was poured into water (20 mL) and extracted with dichloromethane (20 mL×3). The organic phases were concentrated and purified by flash chromatography (silica, petroleum ether/ethyl acetate=½) to give 4-(2-cyanopropan-2-yl)-N-(4-methyl-3-(4,4,5,5-tetramethyl-1,3,2-dioxaborolan-2-yl)phenyl)picolinamide (600 mg, 1.48 mmol, 72%) as a white solid. MS (ESI) m/z 406.2 [M+H]⁺

Step 2. A solution of 3-bromo-N-ethyl-N-(4-methoxybenzyl)-1,6-naphthyridin-7-amine (140 mg, 0.38 mmol), 4-(2-cyanopropan-2-yl)-N-(4-methyl-3-(4,4,5,5-tetramethyl-1,3,2-dioxaborolan-2-yl)phenyl)picolinamide (183 mg, 0.45 mmol), 1,1′-bis(diphenylphosphino)ferrocene-palladium(II)dichloride (31 mg, 0.04 mmol) and potassium carbonate (156 mg, 1.13 mmol) in water (1 mL) and dioxane (5 mL) was stirred at 90° C. for 16 hours under argon. After cooling to room temperature, the mixture was diluted with ethyl acetate (50 mL) and washed with water (50 mL). The organic layer was concentrated and purified by flash chromatography (silica, petroleum ether/ethyl acetate=1/1) give 4-(2-cyanopropan-2-yl)-N-(3-(7-(ethyl(4-methoxybenzyl)amino)-1,6-naphthyridin-3-yl)-4-methylphenyl)picolinamide (60 mg, 0.11 mmol, 29%) as a yellow solid. MS (ESI) m/z 517.2 [M+H]⁺

Step 3. A mixture of 4-(2-cyanopropan-2-yl)-N-(3-(7-(ethyl(4-methoxybenzyl)amino)-1,6-naphthyridin-3-yl)-4-methylphenyl)picolinamide (60 mg, 0.10 mmol) in trifluoroacetic acid (4 mL) was stirred at 45° C. for 2 hours. The reaction was concentrated and the residue was purified by prep-HPLC (Column: Welch Xtimate 21.2×250 mm C18, 10 μm, Mobile Phase: A: water (10 mM ammonium bicarbonate), B: acetonitrile; B %: 30%-70)% in 15 min) to offer 4-(2-cyanopropan-2-yl)-N-(3-(7-(ethylamino)-1,6-naphthyridin-3-yl)-4-methylphenyl)picolinamide (7.2 mg, 0.016 mmol, 15%) as a yellow solid. ¹H NMR (400 MHz, DMSO-d₆) δ 1.22 (t, J=6.8 Hz, 3H), 1.76 (s, 6H), 2.29 (s, 3H), 3.35 (s, 2H), 6.67 (s, 1H), 6.87 (t, J=5.2 Hz, 1H), 7.35 (d, J=8.4 Hz, 1H), 7.83-7.93 (m, 3H), 8.24 (d, J=18.0 Hz, 2H), 8.80 (q, J=5.2 Hz, 2H), 8.98 (s, 1H), 10.73 (s, 1H). MS (ESI) m/z 451.1 [M+H]⁺

### Example 139. Synthesis of N-(3-(7-amino-1,6-naphthyridin-3-yl)-4-methylphenyl)-4-(2-cyanopropan-2-yl)picolinamide (Compound 174)

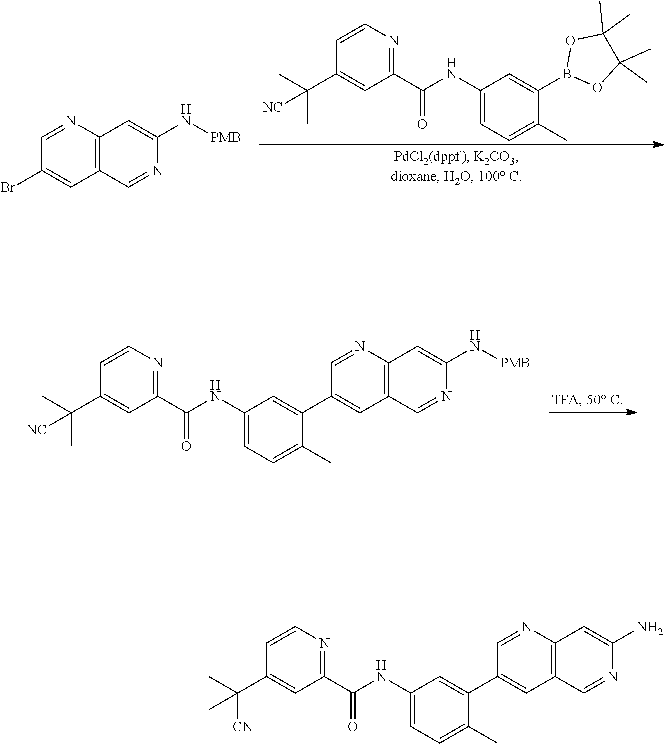

Step 1. A mixture of 3-bromo-N-(4-methoxybenzyl)-1,6-naphthyridin-7-amine (120 mg, 0.35 mmol), 1,1′-bis(diphenylphosphino)ferrocene-palladium(II)dichloride dichloromethane complex (29 mg, 0.038 mmol), potassium carbonate (145 mg, 1.05 mmol) and 4-(2-cyanopropan-2-yl)-N-(4-methyl-3-(4,4,5,5-tetramethyl-1,3,2-dioxaborolan-2-yl)phenyl)picolinamide (168 mg, 0.42 mmol) in dioxane (5 mL) and water (2 mL) was stirred at 100° C. for 2 hours under nitrogen. After cooling to room temperature, the reaction mixture was poured into water (10 mL) and extracted with ethyl acetate (20 mL×3). The combined organic phases were concentrated and purified by flash chromatography (silica, petroleum dichloromethane/ethyl acetate=1/1) to give 4-(2-cyanopropan-2-yl)-N-(3-(7-((4-methoxybenzyl)amino)-1,6-naphthyridin-3-yl)-4-methylphenyl)picolinamide (180 mg, 0.33 mmol, 94%) as a yellow solid. MS (ESI) m/z 543.2 [M+H]⁺

Step 2. A solution of 4-(2-cyanopropan-2-yl)-N-(3-(7-((4-methoxybenzyl)amino)-1,6-naphthyridin-3-yl)-4-methylphenyl)picolinamide (180 mg, 0.33 mmol) in trifluoroacetic acid (6 mL) was stirred at 50° C. for 1 hour. The reaction mixture was cooled to room temperature and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(3-(7-amino-1,6-naphthyridin-3-yl)-4-methylphenyl)-4-(2-cyanopropan-2-yl)picolinamide (40 mg, 0.094 mmol, 28%) as a yellow solid. ¹H NMR (400 MHz, DMSO-d₆) δ 10.74 (s, 1H), 8.94 (s, 1H), 8.80 (dd, J=6.2, 3.8 Hz, 2H), 8.25 (dd, J=16.6, 1.9 Hz, 2H), 7.99-7.86 (m, 2H), 7.84 (dd, J=5.1, 1.9 Hz, 1H), 7.36 (d, J=8.4 Hz, 1H), 6.77 (s, 1H), 6.38 (s, 2H), 2.29 (s, 3H), 1.76 (s, 6H). MS (ESI) m/z 423.1 [M+H]⁺

### Example 140. Synthesis of 4-(2-cyanopropan-2-yl)-N-(5-(7-(ethylamino)-1,6-naphthyridin-3-yl)-6-methylpyridin-3-yl)picolinamide (Compound 175)

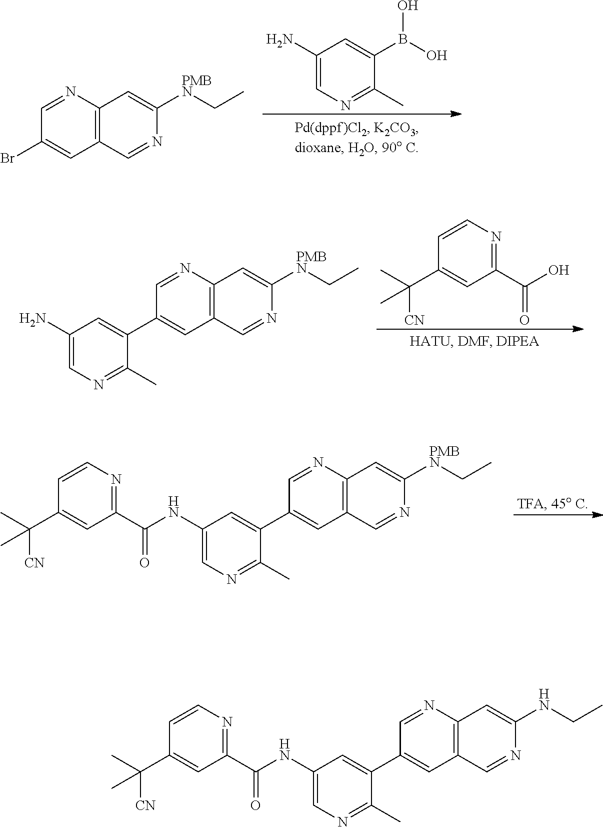

Step 1. A solution of 3-bromo-N-ethyl-N-(4-methoxybenzyl)-1,6-naphthyridin-7-amine (100 mg, 0.37 mmol), (5-amino-2-methylpyridin-3-yl)boronic acid (49 mg, 0.32 mmol), 1,1′-bis(diphenylphosphino)ferrocene-palladium(II)dichloride (22 mg, 0.03 mmol) and potassium carbonate (112 mg, 0.81 mmol) in water (1 mL) and dioxane (5 mL) was stirred at 90° C. for 1 hour under argon. The mixture was diluted with ethyl acetate (50 mL) and washed with water (50 mL). The organic layer was concentrated and purified by flash chromatography (silica, petroleum ether/ethyl acetate=1/1) give 3-(5-amino-2-methylpyridin-3-yl)-N-ethyl-N-(4-methoxybenzyl)-1,6-naphthyridin-7-amine (80 mg, 0.20 mmol, 75%) as a yellow solid. MS (EST) m/z 400.2 [M+H]⁺

Step 2. To a solution of 3-(5-amino-2-methylpyridin-3-yl)-N-ethyl-N-(4-methoxybenzyl)-1,6-naphthyridin-7-amine (80 mg, 0.20 mmol) in N,N-dimethylformamide (3 mL) was added N,N-diisopropylethylamine (78 mg, 0.60 mmol), 1-[bis(dimethylamino)methylene]-1H-1,2,3-triazolo[4,5-b]pyridinium 3-oxid hexafluorophosphate (114 mg, 0.30 mmol) and 4-(2-cyanopropan-2-yl)picolinic acid (38 mg, 0.20 mmol) at room temperature. The mixture was stirred at room temperature for 1 hour. The reaction was diluted with ethyl acetate (100 mL×3), washed with saturated aqueous sodium bicarbonate solution (50 mL) and brine (40 mL), dried over sodium sulfate, filtered, and concentrated. The residue was purified by flash chromatography (silica, petroleum ether/ethyl acetate=1/1) to give 4-(2-cyanopropan-2-yl)-N-(5-(7-(ethyl(4-methoxybenzyl)amino)-1,6-naphthyridin-3-yl)-6-methylpyridin-3-yl)picolinamide (100 mg, 0.175 mmol, 87%) as a yellow solid. MS (ESI) m/z 572.2 [M+H]⁺

Step 3. A mixture of 4-(2-cyanopropan-2-yl)-N-(5-(7-(ethyl(4-methoxybenzyl)amino)-1,6-naphthyridin-3-yl)-6-methylpyridin-3-yl)picolinamide (100 mg, 0.175 mmol) in trifluoroacetic acid (4 mL) was stirred at 45° C. for 2 hours. The reaction was concentrated and the residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to offer 4-(2-cyanopropan-2-yl)-N-(5-(7-(ethylamino)-1,6-naphthyridin-3-yl)-6-methylpyridin-3-yl)picolinamide (36.6 mg, 0.081 mmol, 46%) as a yellow solid. ¹H NMR (400 MHz, DMSO-d₆) δ 1.22 (t, J=7.2 Hz, 3H), 1.77 (s, 6H), 2.50 (s, 3H), 3.34 (s, 2H), 6.68 (s, 1H), 6.93 (t, J=5.2 Hz, 1H), 7.86 (dd, J₁=2.0 Hz, J₂=5.0 Hz, 1H), 8.29 (dd, J₁=2.4 Hz, J₂=6.0 Hz, 3H), 8.81-8.85 (m, 2H), 8.99 (s, 1H), 9.05 (d, J=2.4 Hz, 1H), 11.00 (s, 1H). MS (EST) m/z 452.2 [M+H]⁺

### Example 141. Synthesis of N-(5-(7-amino-1,6-naphthyridin-3-yl)-6-methylpyridin-3-yl)-4-(2-cyanopropan-2-yl)picolinamide (Compound 176)

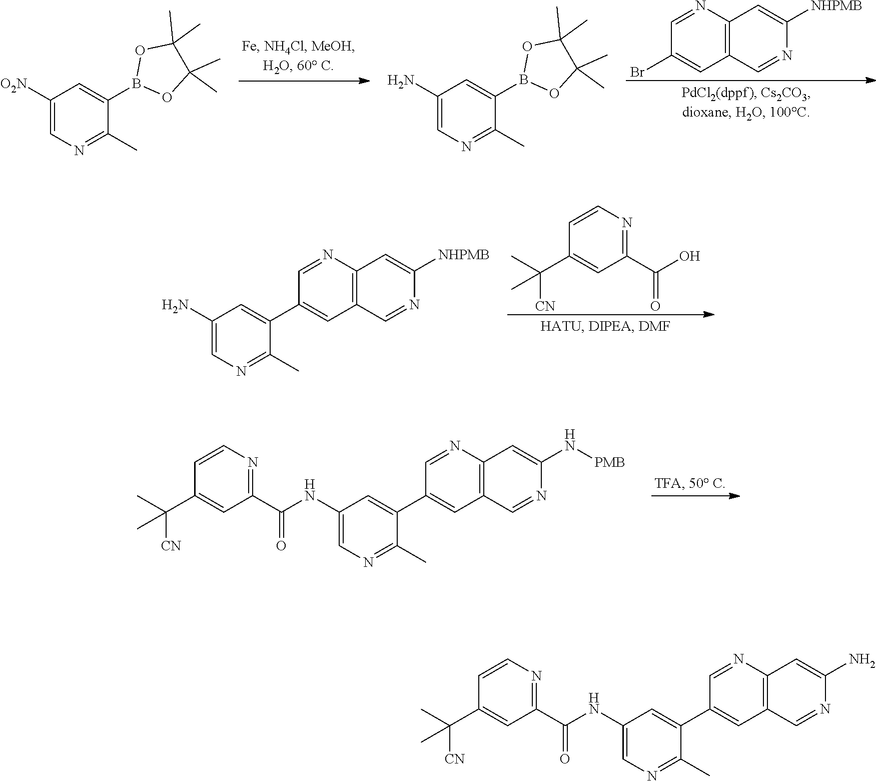

Step 1. A mixture of 2-methyl-5-nitro-3-(4,4,5,5-tetramethyl-1,3,2-dioxaborolan-2-yl)pyridine (1.2 g, 4.54 mmol), iron (1 g, 18.4 mmol) and ammonium chloride (488 mg, 9.2 mmol) in methanol (6 mL) and water (6 mL) was stirred at 60° C. for 0.5 hour. The reaction was filtered and washed with methanol (30 mL). The filtrate was concentrated to give 6-methyl-5-(4,4,5,5-tetramethyl-1,3,2-dioxaborolan-2-yl)pyridin-3-amine (1 g, 4.27 mmol, 94%) as a yellow solid. MS (ESI) m/z 235.1 [M+H]⁺

Step 2. A mixture of 6-methyl-5-(4,4,5,5-tetramethyl-1,3,2-dioxaborolan-2-yl)pyridin-3-amine (235 mg, 1 mmol), 1,1′-bis(diphenylphosphino)ferrocene-palladium(II)dichloride dichloromethane complex (36 mg, 0.044 mmol), cesium carbonate (427 mg, 1.31 mmol) and 3-bromo-N-(4-methoxybenzyl)-1,6-naphthyridin-7-amine (150 mg, 0.437 mmol) in dioxane (5 mL) and water (2 mL) was stirred at 100° C. for 2 hours under nitrogen. After cooling to room temperature, the reaction mixture was poured into water (10 mL) and extracted with ethyl acetate (20 mL×3). The combined organic phases were concentrated and purified by flash chromatography (silica, petroleum methanol/dichloromethane=1/10) to give 3-(5-amino-2-methylpyridin-3-yl)-N-(4-methoxybenzyl)-1,6-naphthyridin-7-amine (100 mg, 0.27 mmol, 61%) as a yellow solid. MS (ESI) m/z 372.1 [M+H]⁺

Step 3. A mixture of 3-(5-amino-2-methylpyridin-3-yl)-N-(4-methoxybenzyl)-1,6-naphthyridin-7-amine (100 mg, 0.27 mmol), 4-(2-cyanopropan-2-yl)picolinic acid (51 mg, 0.27 mmol), 1-[bis(dimethylamino)methylene]-1H-1,2,3-triazolo[4,5-b]pyridinium 3-oxid hexafluorophosphate (153 mg, 0.405 mmol) and N,N-diisopropylethylamine (104 mg, 0.81 mmol) in N,N-dimethylformamide (10 mL) was stirred at room temperature for 0.5 hour. The mixture was poured into water (20 mL) and extracted with dichloromethane (20 mL×3). The organic phases were concentrated and purified by flash chromatography (silica, methanol/dichloromethane=1/20) to give 4-(2-cyanopropan-2-yl)-N-(5-(7-((4-methoxybenzyl)amino)-1,6-naphthyridin-3-yl)-6-methylpyridin-3-yl)picolinamide (120 mg, 0.22 mmol, 82%) as a yellow solid. MS (EST) m/z 544.2 [M+H]⁺

Step 4. A solution of 4-(2-cyanopropan-2-yl)-N-(5-(7-((4-methoxybenzyl)amino)-1,6-naphthyridin-3-yl)-6-methylpyridin-3-yl)picolinamide (120 mg, 0.22 mmol) in trifluoroacetic acid (6 mL) was stirred at 50° C. for 1 hour. The reaction mixture was cooled to room temperature and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(5-(7-amino-1,6-naphthyridin-3-yl)-6-methylpyridin-3-yl)-4-(2-cyanopropan-2-yl)picolinamide (41.8 mg, 0.099 mmol, 45%) as a yellow solid. ¹H NMR (400 MHz, DMSO-d₆) δ 11.02 (s, 1H), 9.06 (d, J=2.4 Hz, 1H), 8.96 (s, 1H), 8.84 (dd, J=12.3, 3.7 Hz, 2H), 8.30 (dd, J=4.9, 2.0 Hz, 3H), 7.86 (dd, 1=5.2, 2.0 Hz, 1H), 6.78 (s, 1H), 6.44 (s, 2H), 2.49 (s, 3H), 1.77 (s, 6H). MS (ESI) m/z 424.2 [M+H]⁺

### Example 142. Synthesis of 1-(4-(difluoromethyl)pyridin-2-yl)-1-methyl-3-(4-methyl-3-(7-(methylamino)-1,6-naphthyridin-3-yl)phenyl)urea (Compound 189)

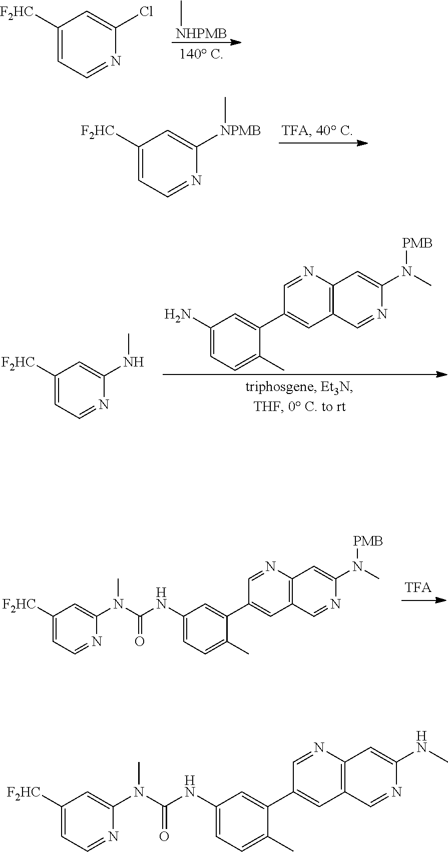

Step 1. A solution of 2-chloro-4-(difluoromethyl)pyridine (450 mg, 2.76 mmol) in 1-(4-methoxyphenyl)-N-methylmethanamine (4 mL) was stirred at 140° C. for 16 hours. The mixture was cooled to room temperature and concentrated. The crude residue was purified by flash chromatography (silica, ethyl acetate/petroleum ether=1/1) to give 4-(difluoromethyl)-N-(4-methoxybenzyl)-N-methylpyridin-2-amine (0.40 g, 1.44 mmol, 52%) as a yellow oil. MS (EST) m/z 279.1 [M+H]⁺

Step 2. A mixture of 4-(difluoromethyl)-N-(4-methoxybenzyl)-N-methylpyridin-2-amine (150 mg, 0.54 mmol) in trifluoroacetic acid (4 mL) was stirred at 40° C. for 2 hours. The mixture was concentrated and purified by flash chromatography (silica, 100% ethyl acetate) to give 4-(difluoromethyl)-N-methylpyridin-2-amine (80 mg, 0.51 mmol, 94%) as a yellow oil. MS (ESI) m/z 159.1 [M+H]⁺

Step 3. To a solution of 4-(difluoromethyl)-N-methylpyridin-2-amine (60 mg, 0.38 mmol) in tetrahydrofuran (8 mL) was added triethylamine (115 mg, 1.14 mmol) and triphosgene (56 mg, 0.19 mmol) at 0° C. After stirring at 0° C. for 30 minutes, 3-(5-amino-2-methylphenyl)-N-(4-methoxybenzyl)-N-methyl-1,6-naphthyridin-7-amine (146 mg, 0.38 mmol) was added and the mixture was stirred at room temperature for 5 hours. The reaction mixture was concentrated and the residue was purified by flash chromatography (silica, ethyl acetate/petroleum ether=3/2) to give 1-(4-(difluoromethyl)pyridin-2-yl)-3-(3-(7-((4-methoxybenzyl)(methyl)amino)-1,6-naphthyridin-3-yl)-4-methylphenyl)-1-methylurea (46 mg, 0.08 mmol, 21%) as a yellow oil. MS (ESI) m/z 568.7 [M+H]⁺

Step 4. A mixture of 1-(4-(difluoromethyl)pyridin-2-yl)-3-(3-(7-((4-methoxybenzyl)(methyl)amino)-1,6-naphthyridin-3-yl)-4-methylphenyl)-1-methylurea (46 mg, 0.08 mmol) in trifluoroacetic acid (4 mL) was stirred at room temperature for 2 hours. The mixture was concentrated and the residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 1-(4-(difluoromethyl)pyridin-2-yl)-1-methyl-3-(4-methyl-3-(7-(methylamino)-1,6-naphthyridin-3-yl)phenyl)urea (23.5 mg, 0.052 mmol, 67%) as a yellow solid. ¹H NMR (500 MHz, DMSO-d₆) δ 11.47 (s, 1H), 8.98 (s, 1H), 8.80 (d, J=2.0 Hz, 1H), 8.60 (d, J=5.5 Hz, 1H), 8.22 (d, J=1.5 Hz, 1H), 7.58-7.55 (m, 3H), 7.31-7.29 (m, 2H), 7.10 (t, J=55.5 Hz, 1H), 6.90-6.87 (m, 1H), 6.62 (s, 1H), 3.45 (s, 3H), 2.87 (d, J=5.0 Hz, 3H), 2.26 (s, 3H). MS (ESI) m/z 449.1 [M+H]⁺

### Example 143. Synthesis of 1-(2-(difluoromethyl)pyridin-4-yl)-1-methyl-3-(6-methyl-5-(7-(methylamino)-1,6-naphthyridin-3-yl)pyridin-3-yl)urea (Compound 190)

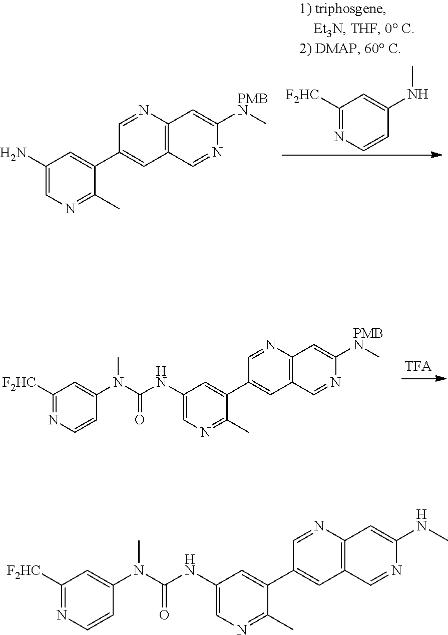

Step 1. 2-(Difluoromethyl)-N-methylpyridin-4-amine (40 mg, 0.25 mmol) was added to a solution of triphosgene (74 mg, 0.25 mmol) and triethylamine (0.1 mL) in tetrahydrofuran (3 mL). The reaction mixture was stirred at 0° C. for 1 hour. Then 3-(5-amino-2-methylpyridin-3-yl)-N-(4-methoxybenzyl)-N-methyl-1,6-naphthyridin-7-amine (80 mg, 0.21 mmol) was added. The reaction mixture was stirred at 60° C. for 1 hour. After cooling to room temperature, the reaction mixture was concentrated and the residue was purified by prep-HPLC (Column: Xbridge 21.2×250 mm C18, 10 μm, Mobile Phase A: water (10 mM ammonium bicarbonate), B: acetonitrile) to give 1-(2-(difluoromethyl)pyridin-4-yl)-3-(5-(7-((4-methoxybenzyl)(methyl)amino)-1,6-naphthyridin-3-yl)-6-methylpyridin-3-yl)-1-methylurea (50 mg, 0.087 mmol, 41%) as a yellow solid. MS (ESI) m/z 570 [M+H]⁺

Step 2. 1-(2-(Difluoromethyl)pyridin-4-yl)-3-(5-(7-((4-methoxybenzyl)(methyl)amino)-1,6-naphthyridin-3-yl)-6-methylpyridin-3-yl)-1-methylurea (50 mg, 0.087 mmol) in trifluoroacetic acid (2 mL) was stirred at room temperature for 1 hour. The reaction mixture was concentrated and the residue was purified by prep-HPLC (Column: Xbridge 21.2×250 mm C18, 10 μm, Mobile Phase A: water (10 mM ammonium bicarbonate), B: acetonitrile) to give 1-(2-(difluoromethyl)pyridin-4-yl)-1-methyl-3-(6-methyl-5-(7-(methylamino)-1,6-naphthyridin-3-yl)pyridin-3-yl)urea (15 mg, 0.033 mmol, 38%) as a yellow solid. ¹H NMR (400 MHz, DMSO-d₆) δ 9.41 (s, 1H), 9.03 (s, 1H), 8.87 (d, J=2.3 Hz, 1H), 8.66 (d, J=2.4 Hz, 1H), 8.60 (d, J=5.6 Hz, 1H). 8.33 (s, 1H), 7.95 (d, J=2.3 Hz, 1H), 7.75 (d, J=2.1 Hz, 1H), 7.57 (d, J=5.6 Hz, 1H), 7.10-6.94 (m, 2H), 6.66 (s, 1H), 3.43 (d, J=4.9 Hz, 3H), 2.91 (d, J=4.7 Hz, 3H), 2.37 (s, 3H). MS (ESI) m/z 450.1 [M+H]⁺

### Example 144. Synthesis of 3-(1-cyanocyclopropyl)-N-(6-methyl-5-(7-(methylamino)-1,6-naphthyridin-3-yl)pyridin-3-yl)benzamide (Compound 191)

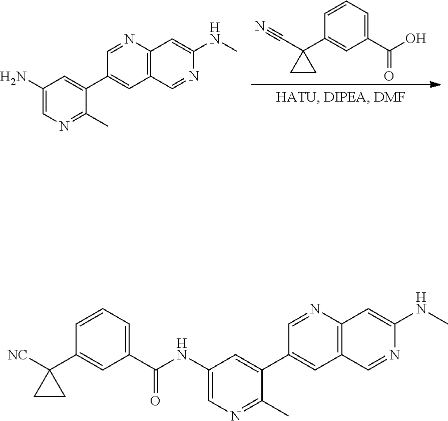

To a solution of 3-(1-cyanocyclopropyl)benzoic acid (30 mg, 0.16 mmol) in N,N-dimethylformamide (5 mL) were added 1-[bis(dimethylamino)methylene]-1H-1,2,3-triazolo[4,5-b]pyridinium 3-oxid hexafluorophosphate (73 mg, 0.19 mmol), N,N-diisopropylethylamine (60 mg, 0.48 mmol) and 3-(5-amino-2-methylpyridin-3-yl)-N-methyl-1,6-naphthyridin-7-amine (42 mg, 0.16 mmol) at room temperature. The reaction mixture was stirred at room temperature for 1 hour. The reaction mixture was diluted with ethyl acetate (100 mL), washed with water (50 mL×2) and brine (50 mL), dried over sodium sulfate, filtered, and concentrated. The crude product was purified by flash chromatography (silica, petroleum ether/ethyl acetate=1/4) to afford 3-(1-cyanocyclopropyl)-N-(6-methyl-5-(7-(methylamino)-1,6-naphthyridin-3-yl)pyridin-3-yl)benzamide (50 mg, 0.09 mmol, 56%) as a yellow oil. ¹H NMR (400 MHz, DMSO-d₆) δ 10.34 (s, 1H), 8.99 (s, 1H), 8.82 (d, J=2.3 Hz, 1H), 8.24 (d, J=1.7 Hz, 1H), 7.96-7.81 (m, 2H), 7.79-7.68 (m, 2H), 7.56 (dd, J=4.8, 1.4 Hz, 2H), 7.35 (d, J=9.1 Hz, 1H), 6.90 (d, J=5.1 Hz, 1H), 6.62 (s, 1H), 2.87 (d, J=5.0 Hz, 3H), 2.31 (d, J=17.1 Hz, 3H), 1.82 (q, J=4.8 Hz, 2H), 1.62 (q, J=5.1 Hz, 2H). MS (ESI) m/z 435 [M+H]⁺

### Example 145. Synthesis of 3-(2-cyanopropan-2-yl)-N-(4-methyl-3-(7-(methylamino)-1,6-naphthyridin-3-yl)phenyl)benzamide (Compound 193)

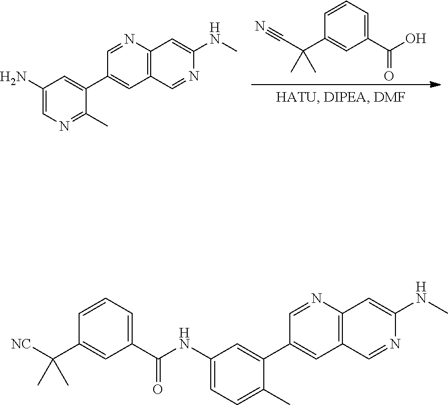

A solution of 3-(2-cyanopropan-2-yl)benzoic acid (30 mg, 0.16 mmol), 3-(5-amino-2-methylphenyl)-N-methyl-1,6-naphthyridin-7-amine (33.5 mg, 0.13 mmol), 1-[bis(dimethylamino)methylene]-1H-1,2,3-triazolo[4,5-b]pyridinium 3-oxid hexafluorophosphate (91.2 mg, 0.24 mmol) and N,N-diisopropylethylamine (61.9 mg, 0.48 mmol) in N,N-dimethylformamide (3 mL) was stirred at room temperature for 2 hours. The reaction mixture was purified by prep-HPLC (Column: Xbridge 21.2×250 mm C18, 10 μm, Mobile Phase A: water (10 mM ammonium bicarbonate), B: acetonitrile) to give 3-(2-cyanopropan-2-yl)-N-(4-methyl-3-(7-(methylamino)-1,6-naphthyridin-3-yl)phenyl)benzamide (11.2 mg, 0.025 mmol, 16%) as a yellow solid. ¹H NMR (400 MHz, CDCl₃) δ 8.87-8.85 (m, 1H), 8.00 (dd, J=16.6, 10.6 Hz, 3H), 7.81 (d, J=7.8 Hz, 1H), 7.70 (d, J=7.6 Hz, 1H), 7.67-7.56 (m, 2H), 7.53 (t, J=7.8 Hz, 1H), 7.34 (d, J=8.2 Hz, 1H), 6.78 (s, 1H), 3.03 (d, J=5.3 Hz, 3H), 2.32 (s, 3H), 1.77 (d, J=5.5 Hz, 6H). MS (EST) m/z 436.0 [M+H]⁺

### Example 146. Synthesis of N-(6-methyl-5-(7-(methylamino)-1,6-naphthyridin-3-yl)pyridin-3-yl)-11H-indole-3-carboxamide (Compound 194)

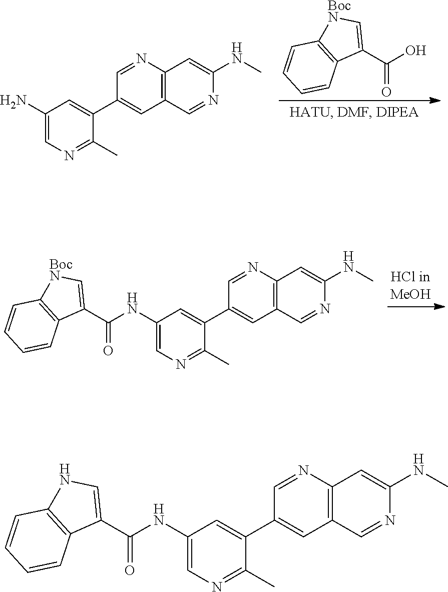

Step 1. A mixture of 1-(tert-butoxycarbonyl)-1H-indole-3-carboxylic acid (30 mg, 0.18 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (84 mg, 0.22 mmol), N,N-dimethylformamide (10 mL) and N,N-diisopropylethylamine (67 mg, 0.54 mmol) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give tert-butyl 3-((6-methyl-5-(7-(methylamino)-1,6-naphthyridin-3-yl)pyridin-3-yl)carbamoyl)-1H-indole-1-carboxylate (70 mg, 0.13 mmol, 72%). MS (ESI) m/z 508 [M+H]⁺

Step 2. A mixture of tert-butyl 3-((6-methyl-5-(7-(methylamino)-1,6-naphthyridin-3-yl)pyridin-3-yl)carbamoyl)-1H-indole-1-carboxylate (50 mg, 0.10 mmol) and hydrogen chloride in methanol solution (3M, 5 mL) was stirred at room temperature for 1 hour, and then the reaction mixture was concentrated. The mixture was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-1H-indole-3-carboxamide (30 mg, 0.13 mmol, 70%). ¹H NMR (400 MHz, DMSO-d₆) δ 11.81 (s, 1H), 9.97 (s, 1H), 9.01 (s, 1H), 8.91-8.84 (m, 2H), 8.33 (d, J=2.4 Hz, 2H), 8.20 (dd, J=10.1, 5.0 Hz, 2H), 7.48 (d, J=7.8 Hz, 1H), 7.25-7.10 (m, 2H), 6.95 (q, J=5.0 Hz, 1H), 6.64 (s, 1H), 2.88 (d, J=5.0 Hz, 3H), 2.54-2.44 (m, 3H). MS (ESI) m/z 409 [M+H]⁺

### Example 147. Synthesis of 7-methyl-N-(4-methyl-3-(7-(methylamino)-1,6-naphthyridin-3-yl)phenyl)-5,6,7,8-tetrahydroimidazo[1,2-a]pyridine-3-carboxamide (Compound 195)

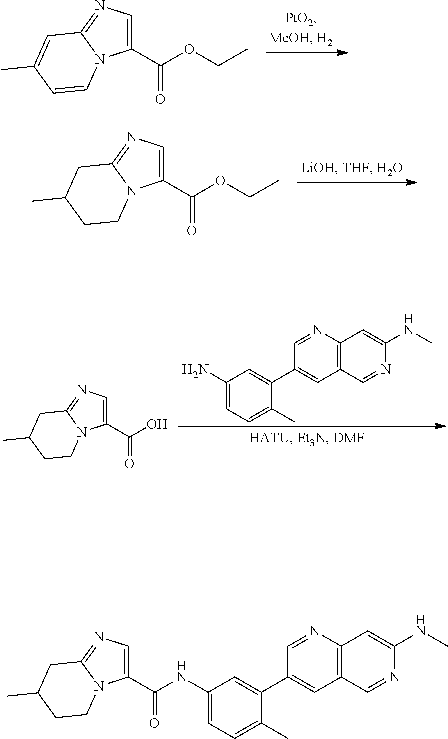

Step 1. To a solution of ethyl 7-methylimidazo[1,2-a]pyridine-3-carboxylate (50 mg, 0.25 mmol) in methanol (3 mL) was added platinum(IV) oxide (6 mg, 0.025 mmol) at room temperature. The mixture was stirred at room temperature for 16 hours under hydrogen atmosphere. The resulting mixture was filtered. The filtrate was concentrated and purified by flash chromatography (silica, ethyl acetate/petroleum ether=1/1) to give ethyl 7-methyl-5,6,7,8-tetrahydroimidazo[1,2-a]pyridine-3-carboxylate (48 mg, 0.23 mmol, 92%) as a colorless oil. MS (ESI) m/z 209.1 [M+H]⁺

Step 2. A mixture of ethyl 7-methyl-5,6,7,8-tetrahydroimidazo[1,2-a]pyridine-3-carboxylate (48 mg, 0.23 mmol) and lithium hydroxide monohydrate (29 mg, 0.69 mmol) in tetrahydrofuran (3 mL) and water (1 mL) was stirred at room temperature for 16 hours. The resulting mixture was diluted with water (10 mL) and acidified with hydrochloric acid to pH=5. The solid was collected by filtration and dried to give 7-methyl-5,6,7,8-tetrahydroimidazo[1,2-a]pyridine-3-carboxylic acid (32 mg, 0.18 mmol, 77%) as white solid. MS (ESI) m/z 181.1 [M+H]⁺

Step 3. A solution of 7-methyl-5,6,7,8-tetrahydroimidazo[1,2-a]pyridine-3-carboxylic acid (32 mg, 0.18 mmol), 3-(5-amino-2-methylphenyl)-N-methyl-1,6-naphthyridin-7-amine (48 mg, 0.18 mmol), 1-[bis(dimethylamino)methylene]-1H-1,2,3-triazolo[4,5-b]pyridinium 3-oxid hexafluorophosphate (205 mg, 0.54 mmol) and triethylamine (0.1 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 7-methyl-N-(4-methyl-3-(7-(methylamino)-1,6-naphthyridin-3-yl)phenyl)-5,6,7,8-tetrahydroimidazo[1,2-a]pyridine-3-carboxamide (18.6 mg, 0.04 mmol, 24%) as a yellow solid. ¹H NMR (400 MHz, DMSO-d₆) δ 9.80 (s, 1H), 8.97 (s, 1H), 8.80 (d, J=2.3 Hz, 1H), 8.22 (d, J=1.8 Hz, 1H), 7.86 (d, J=2.2 Hz, 1H), 7.78 (dd, J=8.3, 2.2 Hz, 1H), 7.68 (s, 1H), 7.27 (d, J=8.5 Hz, 1H), 6.89 (d, J=4.9 Hz, 1H), 6.62 (s, 1H), 4.17-4.10 (m, 1H), 3.98-3.90 (m, 1H), 2.92 (dd, J=16.3, 4.8 Hz, 1H), 2.87 (d, J=4.7 Hz, 3H), 2.38 (dd, J=16.5, 10.5 Hz, 1H), 2.26 (s, 3H), 2.07-1.93 (m, 2H), 1.62 (ddd, J=24.2, 11.1, 5.5 Hz, 1H), 1.08 (d, J=6.6 Hz, 3H). MS (ESI) m/z 427.3 [M+H]⁺

### Example 148. Synthesis of N-(3-(2-cyanopropan-2-yl)phenyl)-6-(7-(methylamino)-1,6-naphthyridin-3-yl)pyridazine-4-carboxamide (Compound 196)

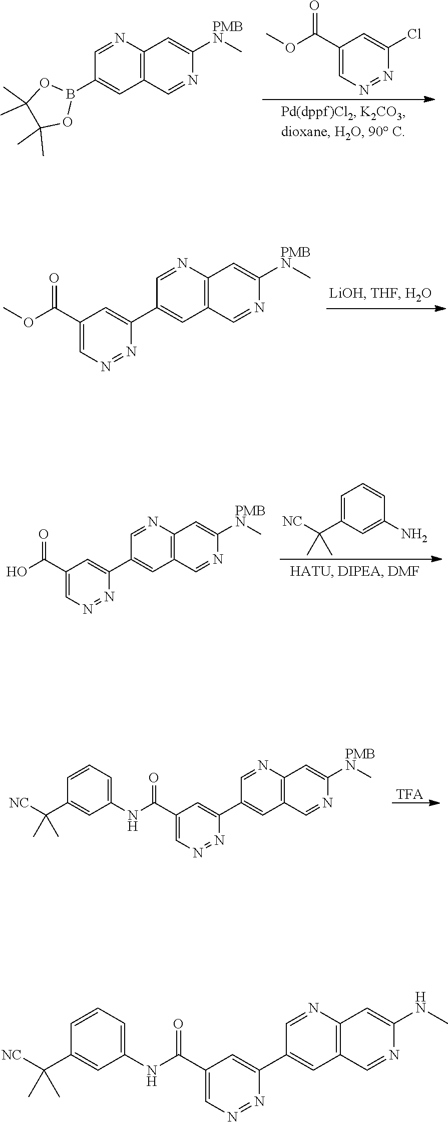

Step 1. A mixture of methyl 6-chloropyridazine-4-carboxylate (212 mg, 1.23 mmol), N-(4-methoxybenzyl)-N-methyl-3-(4,4,5,5-tetramethyl-1,3,2-dioxaborolan-2-yl)-1,6-naphthyridin-7-amine (500 mg, 1.23 mmol), 1,1′-bis(diphenylphosphino)ferrocene]dichloropalladium(II) (106 mg, 0.13 mmol) and potassium carbonate (340 mg, 2.46 mmol) in water (1 mL) and 1,4-dioxane (5 mL) was stirred at 90° C. for 2 hours under nitrogen atmosphere. After cooling to room temperature, the reaction mixture was diluted with water (100 mL) and extracted with ethyl acetate (100 mL×3). The combined organic layers were dried over sodium sulfate, filtered, and concentrated. The residue was purified by flash chromatography (silica, petroleum ether/ethyl acetate=1/1) to give methyl 6-(7-((4-methoxybenzyl)(methyl)amino)-1,6-naphthyridin-3-yl)pyridazine-4-carboxylate (400 mg, 1.32 mmol, 53%) as a yellow solid. MS (ESI) m/z 416.1 [M+H]⁺

Step 2. A solution of methyl 6-(7-((4-methoxybenzyl)(methyl)amino)-1,6-naphthyridin-3-yl)pyridazine-4-carboxylate (100 mg, 0.24 mmol) and lithium hydroxide (67 mg, 1.20 mmol) in tetrahydrofuran (3 mL) and water (2 mL) was stirred for 2 hours at room temperature. The solvent was evaporated under reduced pressure. The residue was diluted with water (5 mL), adjusted to pH=3 by adding 1 N hydrochloric acid slowly, and extracted with ethyl acetate (50 mL×3). The combined organic layers were concentrated in vacuo to give 6-(7-((4-methoxybenzyl)(methyl)amino)-1,6-naphthyridin-3-yl)pyridazine-4-carboxylic acid (80 mg, 0.20 mmol, 83%) as a yellow solid. MS (ESI) m/z 402.1 [M+H]⁺

Step 3. A mixture of 6-(7-((4-methoxybenzyl)(methyl)amino)-1,6-naphthyridin-3-yl)pyridazine-4-carboxylic acid (30 mg, 0.18 mmol), 2-(3-aminophenyl)-2-methylpropanenitrile (30 mg, 0.19 mmol), 1-[bis(dimethylamino)methylene]-1H-1,2,3-triazolo[4,5-b]pyridinium 3-oxid hexafluorophosphate (84 mg, 0.22 mmol), N,N-dimethylformamide (10 mL) and N,N-diisopropylethylamine (67 mg, 0.54 mmol) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give N-(3-(2-cyanopropan-2-yl)phenyl)-6-(7-((4-methoxybenzyl)(methyl)amino)-1,6-naphthyridin-3-yl)pyridazine-4-carboxamide (25 mg, 0.046 mmol, 26%). MS (ESI) m/z 544.2 [M+H]⁺

Step 4. A mixture of N-(3-(2-cyanopropan-2-yl)phenyl)-6-(7-((4-methoxybenzyl)(methyl)amino)-1,6-naphthyridin-3-yl)pyridazine-4-carboxamide (25 mg, 0.046 mmol) and trifluoroacetic acid (5 mL) was stirred at room temperature for 1 hour. The reaction mixture was concentrated. The residue was purified by prep-HPLC (Column: Xbridge 21.2×250 mm C18, 10 μm, Mobile Phase A: water (10 mM ammonium bicarbonate), B: acetonitrile) to give N-(3-(2-cyanopropan-2-yl)phenyl)-6-(7-(methylamino)-1,6-naphthyridin-3-yl)pyridazine-4-carboxamide (13 mg, 0.03 mmol, 66%). ¹H NMR (400 MHz, DMSO-d₆) δ 10.90 (s, 1H), 9.63 (dd, J=19.0, 1.9 Hz, 1H), 9.10 (d, J=4.0 Hz, 1H), 8.79 (d, J=1.8 Hz, 2H), 7.99 (s, 1H), 7.86 (d, J=8.0 Hz, 1H), 7.50 (t, J=7.9 Hz, 1H), 7.34 (d, J=7.6 Hz, 1H), 7.17 (d, J=5.1 Hz, 1H), 6.68 (s, 1H), 2.90 (d, J=4.8 Hz, 3H), 1.72 (s, 6H). MS (ESI) m/z 424.0 [M+H]⁺

### Example 149. Synthesis of N-[4-chloro-6-methyl-5-[7-(methylamino)-1,6-naphthyridin-3-yl]-3-pyridyl]-4-(1-cyano-1-methyl-ethyl)pyridine-2-carboxamide (Compound 197)

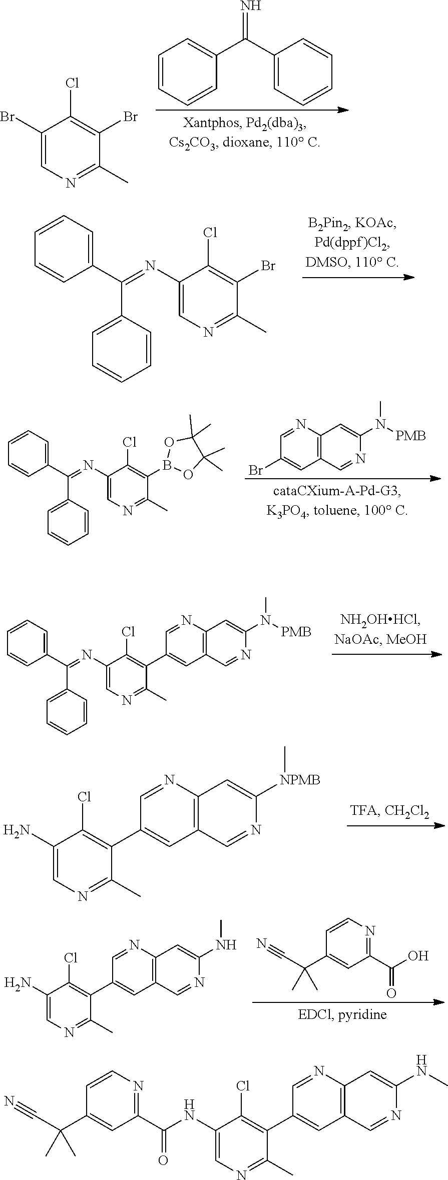

Step 1. To a solution of 3,5-dibromo-4-chloro-2-methyl-pyridine (3.0 g, 10.5 mmol) in dioxane (30 mL) was added diphenylmethanimine (1.91 g, 10.5 mmol), Xantphos (608 mg, 1.05 mmol), tris(dibenzylideneacetone)dipalladium (481 mg, 525 μmol) and cesium carbonate (10.3 g, 31.5 mmol). The mixture was stirred at 110° C. for 12 hours under nitrogen atmosphere. The mixture was concentrated under reduced pressure. The residue was purified by flash chromatography (silica, petroleum ether/ethyl acetate=10/1 to 1/1) to give N-(5-bromo-4-chloro-6-methylpyridin-3-yl)-1,1-diphenylmethanimine (3 g, 7.78 mmol, 74%) as a white solid. MS (ESI) m/z 387.1 [M+H]⁺

Step 2. To a solution of N-(5-bromo-4-chloro-6-methylpyridin-3-yl)-1,1-diphenylmethanimine (2.5 g, 6.48 mmol) in dimethyl sulfoxide (15 mL) was added potassium acetate (1.91 g, 19.5 mmol), 1,1′-bis(diphenylphosphino) ferrocene-palladium(II) dichloride (474 mg, 648 μmol) and 4,4,5,5-tetramethyl-2-(4,4,5,5-tetramethyl-1,3,2-dioxaborolan-2-yl)-1,3,2-dioxaborolane (8.23 g, 32.4 mmol). The mixture was stirred at 110° C. for 8 hours under nitrogen atmosphere. The mixture was then concentrated under vacuum. The residue was purified by flash chromatography (silica, petroleum ether/ethyl acetate=20/1 to 3/1) to give N-[4-chloro-6-methyl-5-(4,4,5,5-tetramethyl-1,3,2-dioxaborolan-2-yl)-3-pyridyl]-1,1-diphenyl-methanimine (1.3 g, 2.97 mmol, 46%) as a yellow oil. MS (ESI) m/z 433.1 [M+H]⁺

Step 3. To a solution of N-[4-chloro-6-methyl-5-(4,4,5,5-tetramethyl-1,3,2-dioxaborolan-2-yl)-3-pyridyl]-1,1-diphenyl-methanimine (350 mg, 808 μmol) and 3-bromo-N-[(4-methoxyphenyl)methyl]-N-methyl-1,6-naphthyridin-7-amine (348 mg, 971 μmol) in toluene (5 mL) was added [(di(1-adamantyl)-butylphosphine)-2-(2′-amino-1,1′-biphenyl)]palladium(II) methanesulfonate (29.5 mg, 40.4 μmol) and potassium phosphate tribasic (1.5 M, 1.62 mL). The mixture was stirred at 60° C. for 1 hour and then stirred at 100° C. for 12 hours under nitrogen atmosphere. The reaction mixture was concentrated under reduced pressure. The residue was purified by flash chromatography (silica, petroleum ether/ethyl acetate=20/1 to 1/1) to give 3-[5-(benzhydrylideneamino)-4-chloro-2-methyl-3-pyridyl]-N-[(4-methoxyphenyl)methyl]-N-methyl-1,6-naphthyridin-7-amine (0.32 g, 493 μmol, 61%) as a white solid. MS (ESI) m/z 584.2 [M+H]⁺

Step 4. To a solution of 3-[5-(benzhydrylideneamino)-4-chloro-2-methyl-3-pyridyl]-N-[(4-methoxyphenyl)methyl]-N-methyl-1,6-naphthyridin-7-amine (0.3 g, 513 μmol) in methanol (5 mL) was added hydroxylamine hydrochloride (53.5 mg, 770 μmol) and sodium acetate (126 mg, 1.54 mmol). The mixture was stirred at 25° C. for 12 hours. On completion, the reaction mixture was concentrated under reduced pressure. The residue was purified by flash chromatography (silica, petroleum ether/ethyl acetate=20/1 to 1/1) to give 3-(5-amino-4-chloro-2-methyl-3-pyridyl)-N-[(4-methoxyphenyl)methyl]-N-methyl-1,6-naphthyridin-7-amine (200 mg, 471 μmol, 92%) as a white solid. MS (EST) m/z 420.3 [M+H]⁺

Step 5. To a solution of 3-(5-amino-4-chloro-2-methyl-3-pyridyl)-N-[(4-methoxyphenyl)methyl]-N-methyl-1,6-naphthyridin-7-amine (170 mg, 405 μmol) in dichloromethane (3 mL) was added trifluoroacetic acid (1.54 g, 13.5 mmol). The mixture was stirred at 25° C. for 2 hours. The reaction mixture was concentrated under reduced pressure. The residue was purified by reversed-phase HPLC (0.1% formic acid) to give 3-(5-amino-4-chloro-2-methyl-3-pyridyl)-N-methyl-1,6-naphthyridin-7-amine (130 mg, 338 μmol, 84%, FA) as a white solid. MS (ESI) m/z 300.1 [M+H]⁺

Step 6. To a solution of 4-(1-cyano-1-methyl-ethyl)pyridine-2-carboxylic acid (143 mg, 751 μmol) in pyridine (3 mL) was added 1-ethyl-3-(3-dimethylaminopropyl)carbodiimide (144 mg, 752 μmol) and 3-(5-amino-4-chloro-2-methyl-3-pyridyl)-N-methyl-1,6-naphthyridin-7-amine (130 mg, 376 μmol). The mixture was stirred at 25° C. for 1 hour and then concentrated under reduced pressure. The residue was purified by prep-HPLC (Column: Phenomenex Luna 150×25 mm C18, 10 μm, Mobile Phase: A: water (formic acid), B: acetonitrile; B %: 28%-58% in 10 min) to give N-[4-chloro-6-methyl-5-[7-(methylamino)-1,6-naphthyridin-3-yl]-3-pyridyl]-4-(1-cyano-1-methyl-ethyl)pyridine-2-carboxamide (18.4 mg, 38.6 μmol, 10%) as a yellow solid. ¹H NMR (400 MHz, DMSO-d₆) δ 10.68 (s, 1H), 9.18 (s, 1H), 8.98 (s, 1H), 8.84 (d, J=5.2 Hz, 1H), 8.72 (d, J=2.4 Hz, 1H), 8.50 (s, 1H), 8.33 (d, J=1.6 Hz, 1H), 8.28 (d, J=2.0 Hz, 1H), 7.94-7.88 (m 1H), 7.00 (q, J=4.8 Hz, 1H), 6.65 (s, 1H), 2.89 (d, J=4.8 Hz, 3H), 2.37 (s, 3H), 1.79 (s, 6H). MS (ESI) m/z 472.0 [M+H]⁺

### Example 150. Synthesis of N-(6-methyl-5-(7-(methylamino)-1,6-naphthyridin-3-yl)pyridin-3-yl)-5-oxo-5,6,7,8-tetrahydronaphthalene-1-carboxamide (Compound 198)

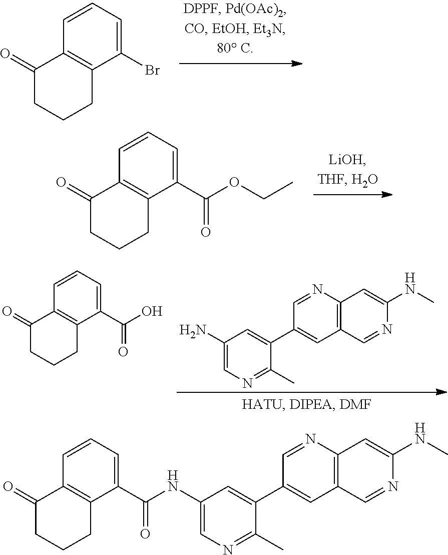

Step 1. A mixture of 5-bromo-3,4-dihydronaphthalen-1(2H)-one (500 mg, 2.22 mmol), 1,1′-bis(diphenylphosphino)ferrocene (246 mg, 0.44 mmol), palladium (II) acetate (49 mg, 0.22 mmol), and triethylamine (2 mL) in ethanol (10 mL) was stirred at 80° C. for 16 hours under carbon monoxide atmosphere. After cooling to room temperature, the reaction mixture was filtered. The filtrate was concentrated under reduced pressure. The residue was purified by flash chromatography (silica, ethyl acetate in petroleum ether from 0% to 20%) to afford ethyl 5-oxo-5,6,7,8-tetrahydronaphthalene-1-carboxylate (460 mg, 2.10 mmol, 95%) as a colorless oil. MS (ESI) m/z 219.1 [M+H]⁺

Step 2. Lithium hydroxide monohydrate (144 mg, 3.44 mmol) and water (1 mL) were added to a solution of ethyl 5-oxo-5,6,7,8-tetrahydronaphthalene-1-carboxylate (150 mg, 0.69 mmol) in tetrahydrofuran (2 mL) at room temperature. The mixture was stirred at room temperature for 1 hour. The resulting mixture was extracted with ethyl acetate (5 mL) and the organic layer was discarded. The aqueous phase was acidified with 1 N hydrogen chloride until pH=3 and extracted with ethyl acetate (5 mL×3). The combined organic phases were washed with brine (10 mL), dried over sodium sulfate, and filtered. The filtrate was concentrated to give 5-oxo-5,6,7,8-tetrahydronaphthalene-1-carboxylic acid (130 mg, crude) as colorless oil, which was used without further purification. MS (ESI) m/z 191.1 [M+H]⁺

Step 3. To a solution of 5-oxo-5,6,7,8-tetrahydronaphthalene-1-carboxylic acid (100 mg, crude) in N,N-dimethylformamide (3 mL) was added N,N-diisopropylethylamine (102 mg, 0.79 mmol), 1-[bis(dimethylamino)methylene]-1H-1,2,3-triazolo[4,5-b]pyridinium 3-oxid hexafluorophosphate (240 mg, 0.63 mmol) at room temperature. The solution was stirred at room temperature for 5 minutes, and then 3-(5-amino-2-methylpyridin-3-yl)-N-methyl-1,6-naphthyridin-7-amine (167 mg, 0.63 mmol) was added. The reaction solution was stirred for 2 hours. The resulting solution was poured into water (10 mL) and extracted with ethyl acetate (10 mL×3). The combined organic phases were washed with brine (10 mL), dried over sodium sulfate, filtered, and concentrated. The residue was purified by flash chromatography (silica, methanol in dichloromethane from 0% to 10%) to give the crude compound (138 mg, 0.32 mmol, 45%). About 15 mg of the crude product was purified by prep-HPLC (Column: Welch Xtimate 21.2×250 mm C18, 10 μm, Mobile Phase: A: water (10 mM ammonium bicarbonate), B: acetonitrile) to give N-(6-methyl-5-(7-(methylamino)-1,6-naphthyridin-3-yl)pyridin-3-yl)-5-oxo-5,6,7,8-tetrahydronaphthalene-1-carboxamide (6.8 mg) as a yellow solid. ¹H NMR (400 MHz, DMSO-d₆) δ 10.72 (s, 1H), 9.01 (s, 1H), 8.86 (d, J=2.4 Hz, 1H), 8.81 (d, J=2.4 Hz, 1H), 8.33 (d, J=2.4 Hz, 1H), 8.17 (d, J=2.4 Hz, 1H), 8.05-8.03 (m, 1H), 7.80-7.77 (m, 1H), 7.52-7.48 (m, 1H), 6.98 (s, 1H), 6.63 (s, 1H), 3.06 (t, J=6.0 Hz, 2H), 2.88 (d, J=4.4 Hz, 3H), 2.67-2.63 (m, 2H), 2.48 (s, 3H), 2.05 (t, J=5.6 Hz, 2H). MS (ESI) m/z 438.1 [M+H]⁺

### Example 151. Synthesis of 5-hydroxy-N-(6-methyl-5-(7-(methylamino)-1,6-naphthyridin-3-yl)pyridin-3-yl)-5,6,7,8-tetrahydronaphthalene-1-carboxamide (Compound 199)

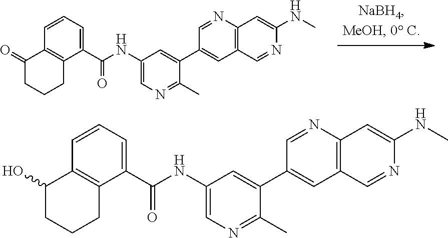 [SMILES: CNc1cc2ncc(-c3cc(NC(=O)c4cccc5c4CCCC5=O)cnc3C)cc2cn1 ; CNc1cc2ncc(-c3cc(NC(=O)c4cccc5c4CCCC5O)cnc3C)cc2cn1]

To a solution of N-(6-methyl-5-(7-(methylamino)-1,6-naphthyridin-3-yl) pyridin-3-yl)-5-oxo-5,6,7,8-tetrahydronaphthalene-1-carboxamide (100 mg, 0.23 mmol) in methanol (5 mL) was added sodium borohydride (9 mg, 0.23 mmol) at 0° C. After stirring at 0° C. for 15 minutes, the reaction mixture was quenched with a solution of ammonium chloride (5 mL) at 0° C. The mixture was concentrated under reduced pressure. The residue was purified by prep-HPLC (Column: Welch Xtimate 21.2×250 mm C18, 10 μm, Mobile Phase: A: water (10 mM ammonium bicarbonate), B: acetonitrile) to give 5-hydroxy-N-(6-methyl-5-(7-(methylamino)-1,6-naphthyridin-3-yl)pyridin-3-yl)-5,6,7,8-tetrahydronaphthalene-1-carboxamide (40.0 mg, 0.09 mmol, 40%) as a yellow solid. ¹H NMR (400 MHz, DMSO-d₆) δ 10.54 (s, 1H), 9.00 (s, 1H), 8.85-8.01 (m, 2H), 8.31 (s, 1H), 8.15 (d, J=2.0 Hz, 1H), 7.55 (d, J=6.8 Hz, 1H), 7.35-7.27 (m, 2H), 7.80-7.77 (m, 1H), 6.97 (d, J=4.0 Hz, 1H), 6.63 (s, 1H), 5.24 (s, 1H), 4.62 (s, 1H), 2.87 (d, J=4.4 Hz, 3H), 2.84-2.70 (m, 2H), 2.47 (s, 3H), 1.94-1.84 (m, 2H), 1.74-1.63 (m, 2H). MS (ESI) m/z 440.1 [M+H]⁺

### Example 152. Synthesis of 3-(1,1-difluoroethyl)-N-(6-methyl-5-(7-(methylamino)-1,6-naphthyridin-3-yl)pyridin-3-yl)benzamide (Compound 200)

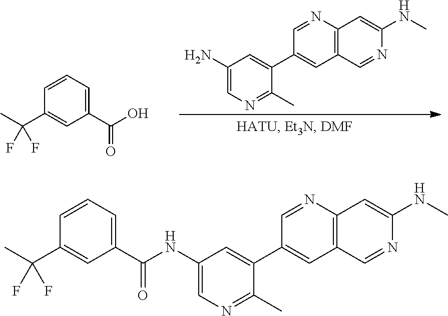 [SMILES: CC(F)(F)c1cccc(C(=O)O)c1 ; CNc1cc2ncc(-c3cc(N)cnc3C)cc2cn1 ; CNc1cc2ncc(-c3cc(NC(=O)c4cccc(C(C)(F)F)c4)cnc3C)cc2cn1]

A solution of 3-(1,1-difluoroethyl)benzoic acid (45 mg, 0.24 mmol), 3-(5-amino-2-methylpyridin-3-yl)-N-methyl-1,6-naphthyridin-7-amine (64 mg, 0.24 mmol), 1-[bis(dimethylamino)methylene]-1H-1,2,3-triazolo[4,5-b]pyridinium 3-oxid hexafluorophosphate (274 mg, 0.72 mmol) and triethylamine (0.2 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3-(1,1-difluoroethyl)-N-(6-methyl-5-(7-(methylamino)-1,6-naphthyridin-3-yl)pyridin-3-yl)benzamide (42.1 mg, 0.10 mmol, 41%) as a yellow solid. ¹H NMR (400 MHz, DMSO-dr) δ 10.63 (s, 1H), 9.00 (s, 1H), 8.91 (d, J=2.4 Hz, 1H), 8.87 (d, J=2.3 Hz, 1H), 8.32 (d, J=2.1 Hz, 1H), 8.18 (d, J=2.3 Hz, 2H), 8.13 (d, J=7.8 Hz, 1H), 7.82 (d, J=7.8 Hz, 1H), 7.69 (t, J=7.8 Hz, 1H), 6.96 (d, J=4.9 Hz, 1H), 6.64 (s, 1H), 2.88 (d, J=4.9 Hz, 3H), 2.50 (s, 3H), 2.04 (t, J=18.9 Hz, 3H). MS (EST) m/z 434.1 [M+H]⁺

### Example 153. Synthesis of 4-(cyclopropyl(hydroxy)methyl)-N-(6-methyl-5-(7-(methylamino)-1,6-naphthyridin-3-yl)pyridin-3-yl)picolinamide (Compound 201)

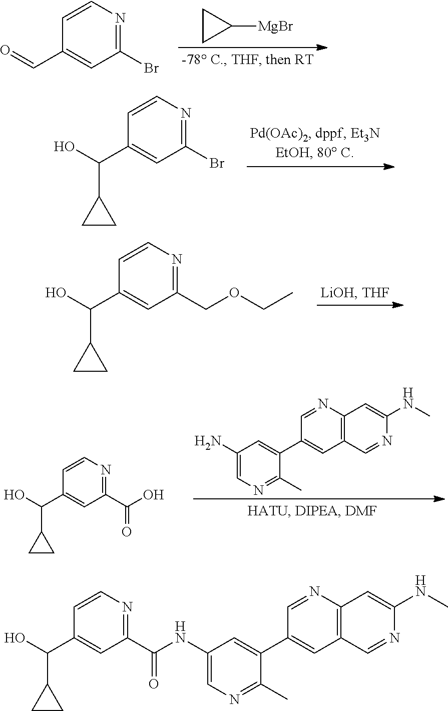

Step 1. To a solution of 2-bromoisonicotinaldehyde (1200 mg, 6.49 mmol) in tetrahydrofuran (30 mL) was added cyclopropylmagnesium bromide (13 ml) at −78° C. under nitrogen atmosphere. The mixture was stirred for 1 hour at room temperature. The reaction mixture was quenched with saturated ammonium chloride aqueous solution (20 mL) and extracted with ethyl acetate (30 mL×2). The combined organic phases were concentrated and purified by flash chromatography (silica, petroleum ether/ethyl acetate=3/1) to give (2-bromopyridin-4-yl)(cyclopropyl)methanol (800 mg, 3.52 mmol, 54%) as colorless oil. MS (ESI) m/z 228 [M+H]⁺

Step 2. A solution of (2-bromopyridin-4-yl)(cyclopropyl)methanol (800 mg, 3.52 mmol), 1,1′-bis(diphenylphosphino)ferrocene (193 mg, 0.35 mmol), palladium (II) acetate (77 mg, 0.35 mmol) and triethylamine (1000 mg, 10.56 mmol) in ethanol (30 mL) was stirred at 80° C. for 16 hours under carbon monoxide atmosphere. The reaction was cooled to room temperature. The mixture were concentrated and purified by flash chromatography (silica, petroleum ether/ethyl acetate=1/1) to give ethyl 4-(cyclopropyl(hydroxy)methyl)picolinate (400 mg, 1.81 mmol, 51%) as a yellow oil. MS (EST) m/z 222 [M+H]⁺

Step 3. A mixture of ethyl 4-(cyclopropyl(hydroxy)methyl)picolinate (200 mg, 0.91 mmol) and lithium hydroxide (76 mg, 1.82 mmol) in tetrahydrofuran (5 mL) was stirred at room temperature for 1 hour. The reaction mixture was extracted with ethyl acetate (30 mL×2) and the organic layers were discarded. The aqueous layer was adjusted to pH=4 with hydrochloric acid (6 N) and extracted with ethyl acetate (30 mL×2). The combined organic phases were concentrated to give 4-(cyclopropyl(hydroxy)methyl)picolinic acid (70 mg, 0.36 mmol, 40%) as a brown oil. MS (ESI) m/z 194 [M+H]⁺

Step 4. A mixture of 4-(cyclopropyl(hydroxy)methyl)picolinic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give 4-(cyclopropyl(hydroxy)methyl)-N-(6-methyl-5-(7-(methylamino)-1,6-naphthyridin-3-yl)pyridin-3-yl)picolinamide (30 mg, 0.068 mmol, 45%). ¹H NMR (400 MHz, DMSO-d₆) δ 10.95 (s, 1H), 9.02 (d, J=23.6 Hz, 1H), 8.99-8.96 (m, 1H), 8.87 (s, 1H), 8.69 (d, J=4.9 Hz, 1H), 8.32 (s, 2H), 8.21 (s, 1H), 7.69 (d, J=4.2 Hz, 1H), 6.96 (d, J=4.9 Hz, 1H), 6.64 (s, 1H), 5.62 (d, J=4.6 Hz, 1H), 4.25-4.07 (m, 1H), 2.88 (d, J=4.7 Hz, 3H), 2.59 (d, J=68.1 Hz, 3H), 1.05 (dd, J=16.9, 9.7 Hz, 1H), 0.48-0.46 (m, 4H). MS (ESI) m/z 441 [M+H]⁺

### Example 154. Synthesis of 4-(1-hydroxy-2-methylpropyl)-N-(6-methyl-5-(7-(methylamino)-1,6-naphthyridin-3-yl)pyridin-3-yl)picolinamide (Compound 202)

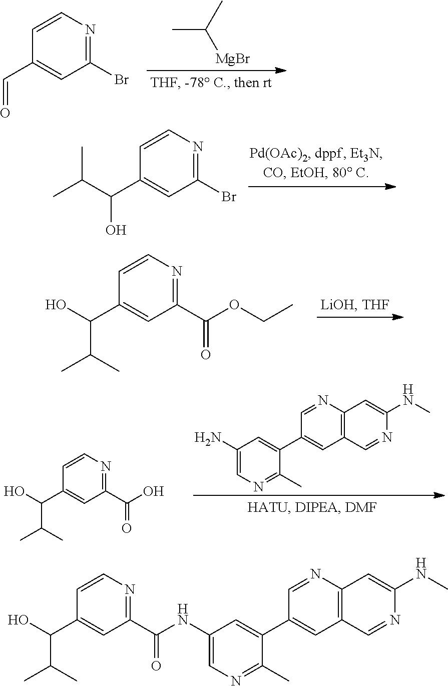

Step 1. To a solution of 2-bromoisonicotinaldehyde (1200 mg, 6.49 mmol) in tetrahydrofuran (30 mL) was added isopropylmagnesium bromide (1 M in tetrahydrofuran, 13 mL, 13 mmol) at −78° C. under nitrogen atmosphere. The mixture was stirred for 1 hour. The reaction mixture was quenched with saturated ammonium chloride aqueous solution (20 mL) and extracted with ethyl acetate (30 mL×2). The combined organic phases were concentrated and purified by flash chromatography (silica, petroleum ether/ethyl acetate=3/1) to give 1-(2-bromopyridin-4-yl)-2-methylpropan-1-ol (800 mg, 3.52 mmol, 54%) as colorless oil. MS (ESI) m/z 230 [M+H]⁺

Step 2. A solution of 1-(2-bromopyridin-4-yl)-2-methylpropan-1-ol (800 mg, 3.52 mmol), 1,1′-bis(diphenylphosphino)ferrocene (193 mg, 0.35 mmol), palladium(II) acetate (77 mg, 0.35 mmol) and triethylamine (1000 mg, 10.56 mmol) in ethanol (30 mL) was stirred at 80° C. for 16 hours under carbon monoxide atmosphere. The reaction was cooled to room temperature. The mixture was concentrated and purified by flash chromatography (silica, petroleum ether/ethyl acetate=1/1) to give ethyl 4-(1-hydroxy-2-methylpropyl)picolinate (400 mg, 1.81 mmol, 51%) as a yellow oil. MS (ESI) m/z 224 [M+H]⁺

Step 3. A mixture of ethyl 4-(1-hydroxy-2-methylpropyl)picolinate (200 mg, 0.91 mmol) and lithium hydroxide (76 mg, 1.82 mmol) in tetrahydrofuran (5 mL) was stirred at room temperature for 1 hour. The reaction mixture was extracted with ethyl acetate (30 mL×2). The organic layers were discarded. The aqueous layer was adjusted to pH=4 with hydrochloric acid (6 N) and extracted with ethyl acetate (30 mL×2). The combined organic phases were concentrated to give 4-(1-hydroxy-2-methylpropyl)picolinic acid (70 mg, 0.36 mmol, 40%) as brown oil. MS (ESI) m/z 196 [M+H]⁺

Step 4. A mixture of 4-(1-hydroxy-2-methylpropyl)picolinic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give 4-(1-hydroxy-2-methylpropyl)-N-(6-methyl-5-(7-(methylamino)-1,6-naphthyridin-3-yl)pyridin-3-yl)picolinamide (30 mg, 0.068 mmol, 45%). ¹H NMR (400 MHz, DMSO-d₆) δ 10.95 (s, 1H), 9.02 (d, J=23.6 Hz, 1H), 8.99-8.96 (m, 1H), 8.87 (s, 1H), 8.69 (d, J=4.9 Hz, 1H), 8.32 (s, 2H), 8.21 (s, 1H), 7.69 (d, J=4.2 Hz, 1H), 6.96 (d, J=4.9 Hz, 1H), 6.64 (s, 1H), 5.62 (d, J=4.6 Hz, 1H), 4.25-4.07 (m, 1H), 2.88 (d, J=4.7 Hz, 3H), 2.59 (d, J=68.1 Hz, 3H), 1.05 (dd, J=16.9, 9.7 Hz, 1H), 0.66-0.24 (m, 6H). MS (ESI) m/z 443 [M+H]⁺

### Example 155. Synthesis of N-(4-methyl-3-(7-(methylamino)-1,6-naphthyridin-3-yl)phenyl)-4-(2,2,2-trifluoro-1-(methylamino)ethyl)picolinamide (Compound 203)

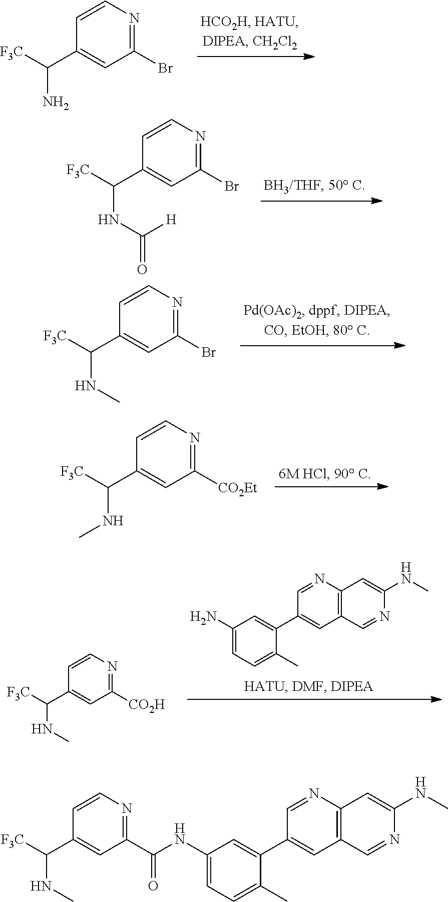

Step 1. A solution of formic acid (1.42 g, 31 mmol), 1-(2-bromopyridin-4-yl)-2,2,2-3% trifluoroethan-1-amine (800 mg, 3.1 mmol), 1-[bis(dimethylamino)methylene]-1H-1,2,3-triazolo[4,5-b]pyridinium 3-oxid hexafluorophosphate (3.02 g, 9.3 mmol) and N,N-diisopropylethylamine (1.2 g, 9.3 mmol) in dichloromethane (50 mL) was stirred at room temperature for 12 hours. The reaction was washed with water (50 mL) and brine (50 mL), dried over sodium sulfate, filtered, and concentrated. The residue was purified by flash chromatography (silica, petroleum ether/ethyl acetated=2/1) to give N-(1-(2-bromopyridin-4-yl)-2,2,2-trifluoroethyl)formamide (300 mg, 1.06 mmol, 34%) as a colorless oil. MS (ESI) m/z 282.9 [M+H]⁺

Step 2. To a solution of N-(1-(2-bromopyridin-4-yl)-2,2,2-trifluoroethyl)formamide (300 mg, 1.06 mmol) in tetrahydrofuran (25 mL) was added borane in tetrahydrofuran (1 M, 11.7 mL, 11.66 mmol) at 0° C. The reaction mixture was stirred at 50° C. for 8 hours. The reaction was quenched with aqueous ammonium chloride (20 mL) and diluted with ethyl acetate (50 mL). The organic layer was washed with water (20 mL) and brine (20 mL), dried over sodium sulfate, filtered, and concentrated. The residue was purified by flash chromatography (silica, petroleum ether/ethyl acetated=1/1) to give 1-(2-bromopyridin-4-yl)-2,2,2-trifluoro-N-methylethan-1-amine (80 mg, 0.30 mmol, 28%) as a yellow solid. MS (EST) m/z 271.0 [M+H]⁺

Step 3. A mixture of 1-(2-bromopyridin-4-yl)-2,2,2-trifluoro-N-methylethan-1-amine (150 mg, 0.56 mmol), palladium(II)acetate (9 mg, 0.0371 mmol), 1,1′-bis(diphenylphosphino)ferrocene (10 mg, 0.0185 mmol) and N,N-diisopropylethylamine (0.5 mL) in ethanol (20 mL) was stirred at 80° C. for 12 hours under the protection of carbon monoxide. After cooling to room temperature, the reaction mixture was concentrated. The residue was diluted with water (20 mL) and extracted with ethyl acetate (15 mL×3). The combined organic phases were washed brine (20 mL×2), dried over sodium sulfate, filtered, and concentrated. The residue was purified by flash chromatography (silica, petroleum ether/ethyl acetate=2/1) to afford ethyl 4-(2,2,2-trifluoro-1-(methylamino)ethyl)picolinate (50 mg, 0.19 mmol, 34%) as a yellow solid. MS (ESI) m/z 264.1 [M+H]⁺

Step 4. A solution of ethyl 4-(2,2,2-trifluoro-1-(methylamino)ethyl)picolinate (50 mg, 0.19 mmol) in 6 M aqueous hydrochloride (5 mL) was stirred at 90° C. for 12 hours. The reaction mixture was concentrated to give 4-(2,2,2-trifluoro-1-(methylamino)ethyl)picolinic acid (20 mg, 0.085 mmol, 45%) as a yellow solid, which was used without further purification. MS (ESI) m/z 235.0 [M+H]⁺

Step 5. A solution of 4-(2,2,2-trifluoro-1-(methylamino)ethyl)picolinic acid (20 mg, 0.085 mmol), 3-(5-amino-2-methylphenyl)-N-methyl-1,6-naphthyridin-7-amine (23 mg, 0.085 mmol), 1-[bis(dimethylamino)methylene]-1H-1,2,3-triazolo[4,5-b]pyridinium 3-oxid hexafluorophosphate (65 mg, 0.17 mmol) and N,N-diisopropylethylamine (0.1 mL) in N,N-dimethylformamide (3 mL) was stirred at room temperature for 12 hours. The reaction was diluted with ethyl acetate (50 mL), washed with water (20 mL) and brine (20 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(4-methyl-3-(7-(methylamino)-1,6-naphthyridin-3-yl)phenyl)-4-(2,2,2-trifluoro-1-(methylamino)ethyl)picolinamide (10 mg, 0.021 mmol, 25%) as a yellow solid. ¹H NMR (400 MHz, DMSO-d₆) δ 10.73 (s, 1H), 9.00 (s, 1H), 8.85 (s, 1H), 8.80 (d, J=4.2 Hz, 1H), 8.31 (s, 2H), 7.93-7.88 (m, 2H), 7.79 (d, J=4.0 Hz, 1H), 7.36 (d, J=8.0 Hz, 1H), 6.98-6.97 (m, 1H), 6.63 (s, 1H), 4.66 (s, 1H), 2.88 (d, J=4.0 Hz, 3H), 2.33 (s, 1H), 2.07 (s, 3H). MS (ESI) m/z 481.0 [M+H]⁺

### Example 156. Synthesis of 6-methyl-N-(4-methyl-3-(7-(methylamino)-1,6-naphthyridin-3-yl)phenyl)-5,6,7,8-tetrahydroimidazo[1,2-a]pyridine-3-carboxamide (Compound 204)

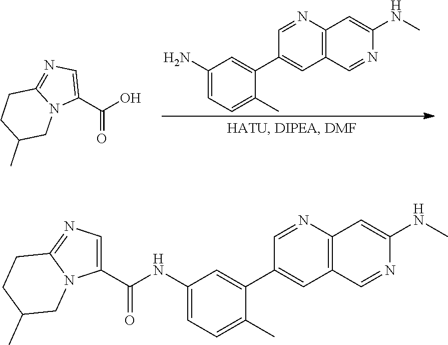 [SMILES: CC1CCc2ncc(C(=O)O)n2C1 ; CNc1cc2ncc(-c3cc(N)ccc3C)cc2cn1 ; CNc1cc2ncc(-c3cc(NC(=O)c4cnc5n4CC(C)CC5)ccc3C)cc2cn1]

To a solution of 3-(5-amino-2-methylphenyl)-N-methyl-1,6-naphthyridin-7-amine (59 mg, 0.22 mmol) in N,N-dimethylformamide (3 mL) was added N,N-diisopropylethylamine (86 mg, 0.67 mmol), 1-[bis(dimethylamino)methylene]-1H-1,2,3-triazolo[4,5-b]pyridinium 3-oxid hexafluorophosphate (127 mg, 0.33 mmol) and 6-methyl-5,6,7,8-tetrahydroimidazo[1,2-a]pyridine-3-carboxylic acid (40 mg, 0.22 mmol). The mixture was stirred at room temperature for 1 hour. The reaction was diluted with ethyl acetate (40 mL), washed with saturated sodium bicarbonate aqueous solution (50 mL) and brine (40 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 6-methyl-N-(4-methyl-3-(7-(methylamino)-1,6-naphthyridin-3-yl)phenyl)-5,6,7,8-tetrahydroimidazo[1,2-a]pyridine-3-carboxamide (29.6 mg, 0.069 mmol, 31%) as a yellow solid. ¹H NMR (500 MHz, DMSO-d₆) δ 1.04 (d, J=6.5 Hz, 3H), 1.46-1.53 (m, 1H), 1.89 (d, J=13.5 Hz, 1H), 2.02 (s, 1H), 2.26 (s, 3H), 2.72-2.79 (m, 1H), 2.87 (d, J=5.0 Hz, 4H), 3.60 (t, J=13.0 Hz, 1H), 4.51 (dd, J₁=5.5 Hz, J₂=13.5 Hz, 1H), 6.62 (s, 1H), 6.89 (q, J=5.0 Hz, 1H), 7.30 (d, J=10.5 Hz, 1H), 7.65 (dd, J₁=2.0 Hz, J₂=8.25 Hz, 1H), 7.71 (s, 1H), 7.77 (s, 1H), 8.21 (s, 1H), 8.80 (d, J=2.5 Hz, 1H), 8.98 (s, 1H), 9.93 (s, 1H). MS (ESI) m/z 427.3 [M+H]⁺

### Example 157. Synthesis of 5-methyl-N-(4-methyl-3-(7-(methylamino)-1,6-naphthyridin-3-yl)phenyl)-5,6,7,8-tetrahydroimidazo[1,2-a]pyridine-3-carboxamide (Compound 205)

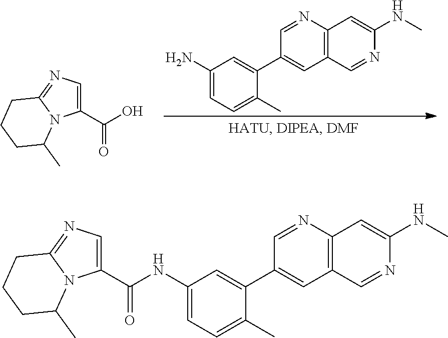 [SMILES: CC1CCCc2ncc(C(=O)O)n21 ; CNc1cc2ncc(-c3cc(N)ccc3C)cc2cn1 ; CNc1cc2ncc(-c3cc(NC(=O)c4cnc5n4C(C)CCC5)ccc3C)cc2cn1]

To a solution of 3-(5-amino-2-methylphenyl)-N-methyl-1,6-naphthyridin-7-amine (81 mg, 0.31 mmol) in N,N-dimethylformamide (3 mL) was added N,N-diisopropylethylamine (180.3 mg, 1.32 mmol), 1-[bis(dimethylamino)methylene]-1H-1,2,3-triazolo[4,5-b]pyridinium 3-oxid hexafluorophosphate (250.8 mg, 0.66 mmol) and 5-methyl-5,6,7,8-tetrahydroimidazo[1,2-a]pyridine-3-carboxylic acid (80 mg, 0.44 mmol). The mixture was stirred at room temperature for 1 hour. The reaction was diluted with ethyl acetate (40 mL), washed with saturated sodium bicarbonate aqueous solution (50 mL) and brine (40 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 5-methyl-N-(4-methyl-3-(7-(methylamino)-1,6-naphthyridin-3-yl)phenyl)-5,6,7,8-tetrahydroimidazo[1,2-a]pyridine-3-carboxamide (4.9 mg, 0.012 mmol, 2.7%) as a yellow solid. ¹H NMR (400 MHz, DMSO-d₆) δ 10.07 (s, 1H), 8.99 (s, 1H), 8.81 (s, 1H), 8.23 (s, 1H), 7.88 (s, 1H), 7.68 (d, J=8.2 Hz, 2H), 7.32 (d, J=7.9 Hz, 1H), 6.95 (d, J=31.0 Hz, 1H), 6.62 (s, 1H), 5.09 (s, 1H), 2.87 (d, J=4.3 Hz, 5H), 2.27 (s, 3H), 2.02 (d, J=40.9 Hz, 2H), 1.83 (d, J=9.7 Hz, 2H), 1.33 (d, J=6.3 Hz, 3H). MS (EST) m/z 427.2 [M+H]⁺

### Example 158. Synthesis of N-(6-methyl-5-(7-(methylamino)-1,6-naphthyridin-3-yl)pyridin-3-yl)-4-(2,2,2-trifluoro-1,1-dihydroxyethyl)picolinamide (Compound 206)

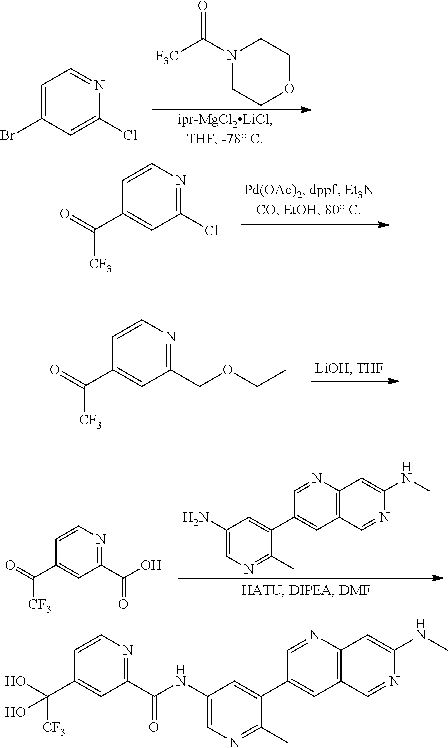

Step 1. A solution of 4-bromo-2-chloropyridine (1200 mg, 6.49 mmol) in tetrahydrofuran (30 mL) was added isopropyl magnesium chloride lithium chloride complex (1.3 M in tetrahydrofuran, 5 mL, 6.5 mmol) at −78° C. in nitrogen atmosphere. The mixture was stirred for 1 hour. Then 2,2,2-trifluoro-1-morpholinoethan-1-one (200 mg, 1.0 mmol) was added and further stirred for 1 hour. The reaction mixture was quenched with saturated ammonium chloride aqueous solution (20 mL) and extracted with ethyl acetate (30 mL×2). The combined organic phases were concentrated and purified by flash chromatography (silica, petroleum ether/ethyl acetate=3/1) to give 1-(2-chloropyridin-4-yl)-2,2,2-trifluoroethan-1-one (800 mg, 3.52 mmol, 54%) as colorless oil. MS (ESI) m/z 210 [M+H]⁺

Step 2. A solution of 1-(2-chloropyridin-4-yl)-2,2,2-trifluoroethan-1-one (800 mg, 3.52 mmol), 1,1′-bis(diphenylphosphino)ferrocene (193 mg, 0.35 mmol), palladium (II) acetate (77 mg, 0.35 mmol) and triethylamine (1000 mg, 10.56 mmol) in ethanol (30 mL) was stirred at 80° C. for 16 hours under carbon monoxide atmosphere. The reaction was cooled to room temperature. The mixture was concentrated and purified by flash chromatography (silica, petroleum ether/ethyl acetate=1/1) to give ethyl 4-(2,2,2-trifluoroacetyl)picolinate (400 mg, 1.81 mmol, 51%) as a yellow oil. MS (ESI) m/z 248 [M+H]⁺

Step 3. A mixture of ethyl 4-(2,2,2-trifluoroacetyl)picolinate (200 mg, 0.91 mmol) and lithium hydroxide (76 mg, 1.82 mmol) in tetrahydrofuran (5 mL) was stirred at room temperature for 1 hour. The reaction mixture was extracted with ethyl acetate (30 mL×2) and the organic layers were discarded. The aqueous layer was adjusted pH=4 with hydrochloric acid (6 N) and extracted with ethyl acetate (30 mL×2). The combined organic phases were concentrated to give 4-(2,2,2-trifluoroacetyl)picolinic acid (70 mg, 0.36 mmol, 40%) as a brown oil. MS (ESI) m/z 220 [M+H]⁺

Step 4. A mixture of 4-(2,2,2-trifluoroacetyl)picolinic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol) and N,N-diisopropylethylamine (67 mg, 0.54 mmol) in N,N-dimethylformamide (10 mL) at room temperature. The mixture was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-4-(2,2,2-trifluoro-1,1-dihydroxyethyl)picolinamide (30 mg, 0.068 mmol, 45%). ¹H NMR (400 MHz, DMSO-d₆) δ 11.03 (s, 1H), 9.05 (d, J=2.3 Hz, 1H), 8.99 (s, 1H), 8.86 (dd, J=7.9, 3.7 Hz, 2H), 8.33 (dd, J=9.9, 7.1 Hz, 3H), 8.13 (s, 2H), 7.86 (d, J=3.8 Hz, 1H), 6.96 (d, J=5.0 Hz, 1H), 6.64 (s, 1H), 2.88 (d, J=4.9 Hz, 3H), 2.55 (m, 3H). MS (ESI) m/z 485 [M+H]⁺

### Example 159. Synthesis of (S)-5-hydroxy-N-(6-methyl-5-(7-(methylamino)-1,6-naphthyridin-3-yl)pyridin-3-yl)-5,6,7,8-tetrahydronaphthalene-1-carboxamide and (R)-5-hydroxy-N-(6-methyl-5-(7-(methylamino)-1,6-naphthyridin-3-yl)pyridin-3-yl)-5,6,7,8-tetrahydronaphthalene-1-carboxamide (Compounds 207 and 208)

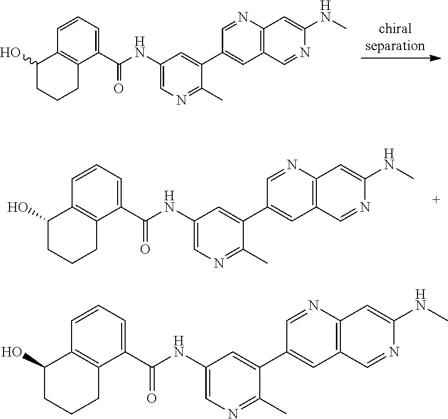 [SMILES: CNc1cc2ncc(-c3cc(NC(=O)c4cccc5c4CCCC5O)cnc3C)cc2cn1 ; CNc1cc2ncc(-c3cc(NC(=O)c4cccc5c4CCC[C@@H]5O)cnc3C)cc2cn1 ; CNc1cc2ncc(-c3cc(NC(=O)c4cccc5c4CCC[C@H]5O)cnc3C)cc2cn1]

The enantiomers of 5-hydroxy-N-(6-methyl-5-(7-(methylamino)-1,6-naphthyridin-3-yl)pyridin-3-yl)-5,6,7,8-tetrahydronaphthalene-1-carboxamide (40 mg, 0.09 mmol) were separated by chiral SFC (Instrument: SFC-150 (Waters); Column: OZ 20×250 mm, 10 μm (Daicel); Mobile phase: CO₂/MeOH (0.2% methanol ammonia)=45/55; Detection wavelength: 214 nm) to afford the first eluting enantiomer (retention time 2.2 min) which was arbitrarily assigned as (S)-5-hydroxy-N-(6-methyl-5-(7-(methylamino)-1,6-naphthyridin-3-yl)pyridin-3-yl)-5,6,7,8-tetrahydronaphthalene-1-carboxamide (8.0 mg, 0.02 mmol) as a yellow solid. ¹H NMR (400 MHz, DMSO-d₆) δ 10.54 (s, 1H), 9.00 (s, 1H), 8.84 (d, J=2.4 Hz, 1H), 8.80 (d, J=2.4 Hz, 1H), 8.30 (d, J=2.0 Hz, 1H), 8.15 (d, J=2.4 Hz, 1H), 7.55 (d, J=6.8 Hz, 1H), 7.36-7.27 (m, 2H), 6.98-6.94 (m, 1H), 6.63 (s, 1H), 5.25 (d, J=5.6 Hz, 1H), 4.61 (d, J=4.0 Hz, 1H), 2.87 (d, J=4.8 Hz, 3H), 2.84-2.71 (m, 2H), 2.46 (s, 3H), 1.93-1.86 (m, 2H), 1.74-1.63 (m, 2H). MS (ESI) m/z 440.1 [M+H]⁺;
and the second eluting enantiomer (retention time 4.5 min) which was arbitrarily assigned as (R)-5-hydroxy-N-(6-methyl-5-(7-(methylamino)-1,6-naphthyridin-3-yl)pyridin-3-yl)-5,6,7,8-tetrahydronaphthalene-1-carboxamide (8.2 mg, 0.002 mmol) as a yellow solid. ¹H NMR (400 MHz, DMSO-d₆) δ 10.60 (s, 1H), 9.01 (s, 1H), 8.86 (d, J=1.2 Hz, 1H), 8.81 (d, J=1.6 Hz, 1H), 8.35 (s, 1H), 8.17 (d, J=2.0 Hz, 1H), 7.56 (d, J=6.8 Hz, 1H), 7.35-7.34 (m, 1H), 7.31-7.28 (m, 1H), 7.02 (s, 1H), 6.63 (s, 1H), 5.25 (br s, 1H), 4.62-4.60 (m, 1H), 2.87 (s, 3H), 2.84-2.72 (m, 2H), 2.47 (s, 3H), 1.92-1.87 (m, 2H), 1.71-1.65 (m, 2H). MS (ESI) m/z 440.1 [M+H]⁺

### Example 160. Synthesis of N-(6-methyl-5-(7-(methylamino)-1,6-naphthyridin-3-yl)pyridin-3-yl)-5,6,7,8-tetrahydroimidazo[1,2-a]pyridine-3-carboxamide (Compound 209)

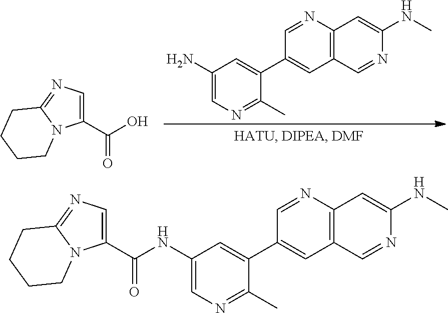 [SMILES: O=C(O)c1cnc2n1CCCC2 ; CNc1cc2ncc(-c3cc(N)cnc3C)cc2cn1 ; CNc1cc2ncc(-c3cc(NC(=O)c4cnc5n4CCCC5)cnc3C)cc2cn1]

A mixture of 5,6,7,8-tetrahydroimidazo[1,2-a]pyridine-3-carboxylic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 24 hours. The mixture was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-5,6,7,8-tetrahydroimidazo[1,2-a]pyridine-3-carboxamide (30 mg, 0.068 mmol, 45%). ¹H NMR (400 MHz, DMSO-d₆) δ 10.14 (s, 1H), 9.01 (d, J=17.5 Hz, 1H), 8.83 (dd, J=14.7, 2.4 Hz, 2H), 8.30 (d, J=1.7 Hz, 1H), 8.09 (d, J=2.4 Hz, 1H), 7.81 (s, 1H), 6.95 (q, J=4.9 Hz, 1H), 6.63 (s, 1H), 4.24 (t, J=5.9 Hz, 2H), 2.93 (dd, J=44.8, 19.8 Hz, 3H), 2.80 (t, J=6.3 Hz, 2H), 2.54-2.44 (m, 3H), 1.93 (t, J=18.2 Hz, 2H), 1.87-1.62 (m, 2H). MS (ESI) m/z 414 [M+H]⁺

### Example 161. Synthesis of N-(6-methyl-5-(7-(methylamino)-1,6-naphthyridin-3-yl)pyridin-3-yl)-6-(2,2,2-trifluoro-1-hydroxyethyl)picolinamide (Compound 210)

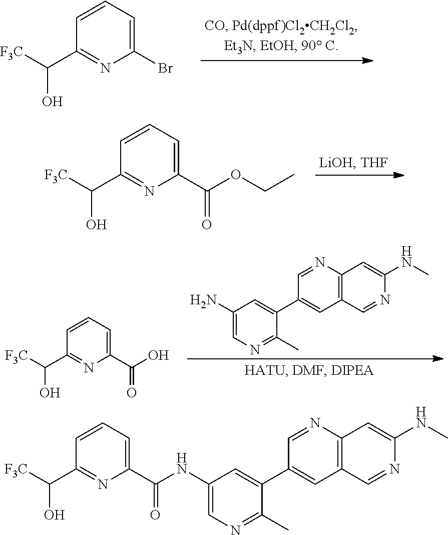

Step 1. A solution of 1-(6-bromopyridin-2-yl)-2,2,2-trifluoroethan-1-ol (280 mg, 1.09 mmol), 1,1′-bis(diphenylphosphino)ferrocene-palladium(II)dichloride dichloromethane complex (90 mg, 0.11 mmol) and triethylamine (330 mg, 3.27 mmol) in ethanol (15 mL) was stirred at 90° C. for 16 hours under carbon monoxide atmosphere. The reaction was cooled to room temperature and concentrated. The residue was quenched with water (15 mL) and extracted with ethyl acetate (30 mL×2). The combined organic phases were concentrated and purified by flash chromatography (silica, petroleum ether/ethyl acetate=1/1) to give ethyl 6-(2,2,2-trifluoro-1-hydroxyethyl)picolinate (200 mg, 0.80 mmol, 74%) as white solid. MS (ESI) m/z 250 [M+H]⁺

Step 2. A mixture of ethyl 6-(2,2,2-trifluoro-1-hydroxyethyl)picolinate (200 mg, 0.80 mmol) and lithium hydroxide (61 mg, 1.6 mmol) in tetrahydrofuran (5 mL) was stirred at room temperature for 1 hour. The reaction mixture was extracted with ethyl acetate (30 mL×2) and the organic layers were discarded. The aqueous layer was adjusted pH=4 with hydrochloric acid (6 N) and extracted with ethyl acetate (30 mL×2). The combined organic phases were concentrated to give 6-(2,2,2-trifluoro-1-hydroxyethyl)picolinic acid (150 mg, 0.68 mmol, 85%) as a brown solid. MS (EST) m/z 222 [M+H]⁺

Step 3. A mixture of 6-(2,2,2-trifluoro-1-hydroxyethyl)picolinic acid (30 mg, 0.14 mmol), 3-(5-amino-2-methylpyridin-3-yl)-N-methyl-1,6-naphthyridin-7-amine (36 mg, 0.14 mmol), 1-[bis(dimethylamino)methylene]-1H-1,2,3-triazolo[4,5-b]pyridinium 3-oxid hexafluorophosphate (106 mg, 0.28 mmol) and N,N-diisopropylethylamine (54 mg, 0.42 mmol) in N,N-dimethylformamide (3 mL) was stirred at room temperature for 1 hour. The reaction mixture was purified by prep-HPLC to give N-(6-methyl-5-(7-(methylamino)-1,6-naphthyridin-3-yl)pyridin-3-yl)-6-(2,2,2-trifluoro-1-hydroxyethyl)picolinamide (18 mg, 0.038 mmol, 27%) as a yellow solid. ¹H NMR (400 MHz, DMSO-d₆) δ 10.94 (s, 1H), 9.03-9.01 (m, 2H), 8.91 (d, J=2.2 Hz, 1H), 8.38 (s, 1H), 8.24 (d, J=2.2 Hz, 1H), 8.19 (dd, J=12.2, 4.8 Hz, 2H), 7.85 (d, J=8.0 Hz, 1H), 7.08 (d, J=8.4 Hz, 1H), 7.02 (s, 1H), 6.64 (s, 1H), 5.49-5.44 (m, 1H), 2.88 (d, J=3.0 Hz, 1H), 2.52 (s, 3H). MS (ESI) m/z 469 [M+H]⁺

### Example 162. Synthesis of 4-hydroxy-N-(6-methyl-5-(7-(methylamino)-1,6-naphthyridin-3-yl)pyridin-3-yl)-3,4-dihydro-2H-pyrano[2,3-c]pyridine-8-carboxamide (Compound 211)

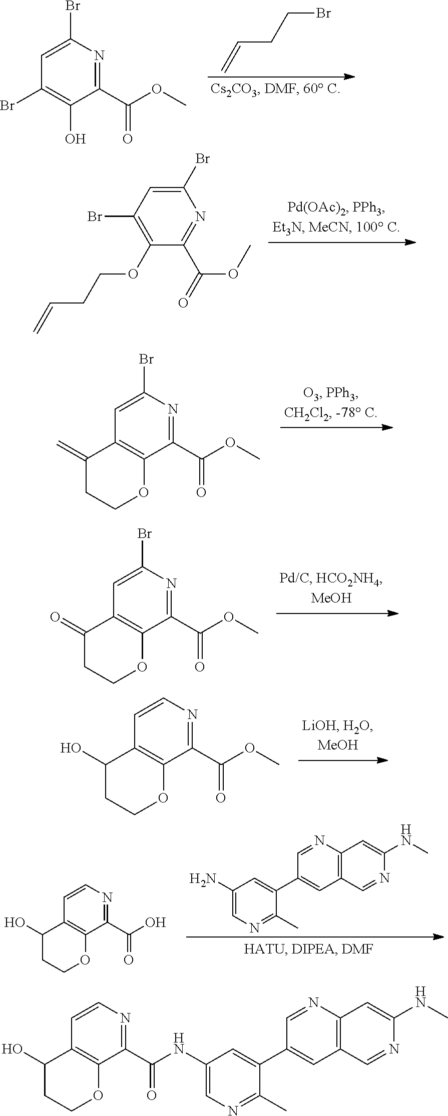

Step 1. A mixture of methyl 4,6-dibromo-3-hydroxypicolinate (2.2 g, 7.07 mmol), 4-bromobut-1-ene (1.24 g, 9.2 mmol) and cesium carbonate (4.6 g, 14.14 mmol) in N,N-dimethylformamide (30 mL) was stirred at 60° C. for 3 hours. The reaction mixture was cooled to room temperature, treated with water (30 mL) and extracted with ethyl acetate (40 mL×3). The combined organic phases were concentrated. The residue was purified by flash chromatography (silica, petroleum ether/ethyl acetate=8/1) to give methyl 4,6-dibromo-3-(but-3-en-1-yloxy)picolinate (2.2 g, 6.02 mmol, 85%) as a yellow solid. MS (ESI) m/z 365.9 [M+H]⁺

Step 2. A mixture of methyl 4,6-dibromo-3-(but-3-en-1-yloxy)picolinate (2.2 g, 6.02 mmol), palladium (II) acetate (403 mg, 1.8 mmol), triphenylphosphine (473 mg, 1.8 mmol) and triethylamine (1818 mg, 18.0 mmol) in acetonitrile (5 mL) was stirred at 100° C. for 2 hours. The reaction mixture was cooled to room temperature and concentrated. The residue was purified by flash chromatography (silica, petroleum ether/ethyl acetate=10/2) to give methyl 6-bromo-4-methylene-3,4-dihydro-2H-pyrano[2,3-c]pyridine-8-carboxylate (400 mg, 1.4 mmol, 23%) as a yellow solid. MS (ESI) m/z 284.0 [M+H]⁺

Step 3. To a solution of methyl 6-bromo-4-methylene-3,4-dihydro-2H-pyrano[2,3-c]pyridine-8-carboxylate (400 mg, 1.4 mmol) in dichloromethane (10 mL) cooled to −78° C. was bubbled ozone until the solution maintained a slight blue color. Nitrogen gas was then bubbled into the solution until it turned clear. Triphenylphosphine (318 mg, 1.22 mmol) was added to the solution and the resulting mixture was stirred at room temperature for 30 minutes. The reaction mixture was concentrated. The residue was purified by flash chromatography (silica, petroleum ether/ethyl acetate=4/1) to give methyl 6-bromo-4-oxo-3,4-dihydro-2H-pyrano[2,3-c]pyridine-8-carboxylate (300 mg, 1.04 mmol, 74%) as a white solid. MS (ESI) m/z 286.0 [M+H]⁺

Step 4. 10% Palladium on carbon (40 mg) and ammonium formate (40 mg). were added to a solution of methyl 6-bromo-4-oxo-3,4-dihydro-2H-pyrano[2,3-c]pyridine-8-carboxylate (120 mg, 0.42 mmol) in methanol (10 mL). The mixture was stirred at 20° C. for 2 hours. The reaction mixture was concentrated and purified by flash chromatography (silica, methanol/dichloromethane=3/97) to give methyl 4-hydroxy-3,4-dihydro-2H-pyrano[2,3-c]pyridine-8-carboxylate (84 mg, 0.4 mmol, 95%) as a yellow oil. MS (ESI) m/z 210.1 [M+H]⁺

Step 5. A mixture of methyl 4-hydroxy-3,4-dihydro-2H-pyrano[2,3-c]pyridine-8-carboxylate (84 mg, 0.4 mmol) and lithium hydroxide monohydrate (84 mg, 2.0 mmol) in water (1 mL) and methanol (10 mL) was stirred at room temperature for 2 hours. The reaction mixture was adjusted to pH=2 with hydrochloric acid (1 N) and concentrated to give 4-hydroxy-3,4-dihydro-2H-pyrano[2,3-c]pyridine-8-carboxylic acid (crude) as a yellow solid. MS (ESI) m/z 196.0 [M+H]⁺

Step 6. A mixture of 4-hydroxy-3,4-dihydro-2H-pyrano[2,3-c]pyridine-8-carboxylic acid (crude), 3-(5-amino-2-methylpyridin-3-yl)-N-methyl-1,6-naphthyridin-7-amine (85 mg, 0.32 mmol), 1-[bis(dimethylamino)methylene]-1H-1,2,3-triazolo[4,5-b]pyridinium 3-oxid hexafluorophosphate (456 mg, 1.2 mmol) and N,N-diisopropylethylamine (155 mg, 1.2 mmol) in N,N-dimethylformamide (5 mL) was stirred at room temperature for 2 hours. The reaction mixture was treated with water (30 mL) and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 4-hydroxy-N-(6-methyl-5-(7-(methylamino)-1,6-naphthyridin-3-yl)pyridin-3-yl)-3,4-dihydro-2H-pyrano[2,3-c]pyridine-8-carboxamide (50 mg, 0.11 mmol, 28%) as a yellow solid. ¹H NMR (500 MHz, DMSO-d₆) δ 10.64 (s, 1H), 9.00 (s, 1H), 8.86 (t, J=2.4 Hz, 2H), 8.32 (s, 1H), 8.18 (dd, J=14.7, 3.4 Hz, 2H), 7.52 (d, J=4.7 Hz, 1H), 6.95 (s, 1H), 6.63 (s, 1H), 5.80 (d, J=5.2 Hz, 1H), 4.71 (s, 1H), 4.32 (t, J=6.0 Hz, 2H), 2.88 (d, J=4.7 Hz, 3H), 2.47 (s, 3H), 2.15-2.05 (m, 1H), 1.98-1.87 (m, 1H). MS (ESI) m/z 443.1 [M+H]⁺

### Example 163. Synthesis of N-(6-methyl-5-(7-(methylamino)-1,6-naphthyridin-3-yl)pyridin-3-yl)-1-oxo-2,3-dihydro-1H-indene-4-carboxamide (Compound 212)

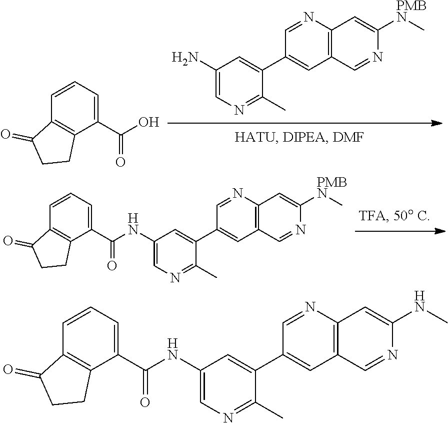

Step 1. To a solution of 1-oxo-2,3-dihydro-1H-indene-4-carboxylic acid (250 mg) in N,N-dimethylformamide (5 mL) was added N,N-diisopropylethylamine (550 mg, 4.26 mmol), 1-[bis(dimethylamino)methylene]-1H-1,2,3-triazolo[4,5-b]pyridinium 3-oxid hexafluorophosphate (1.07 g, 2.84 mmol) at room temperature. The solution was stirred for 5 minutes, and then 3-(5-amino-2-methylpyridin-3-yl)-N-(4-methoxybenzyl)-N-methyl-1,6-naphthyridin-7-amine (546 mg, 1.42 mmol) was added. The reaction solution was stirred at room temperature overnight. The resulting solution was poured into water (10 mL) and extracted with ethyl acetate (10 mL×3). The combined organic phases were washed with brine (10 mL), dried over sodium sulfate, filtered, and concentrated. The residue was purified by flash chromatography (silica, methanol in dichloromethane from 0% to 10%) to give N-(5-(7-((4-methoxybenzyl)(methyl)amino)-1,6-naphthyridin-3-yl)-6-methylpyridin-3-yl)-1-oxo-2,3-dihydro-1H-indene-4-carboxamide (300 mg, 0.55 mmol, 38%) as a yellow solid. MS (ESI) m/z 544.2 [M+H]⁺

Step 2. A solution of N-(5-(7-((4-methoxybenzyl)(methyl)amino)-1,6-naphthyridin-3-yl)-6-methylpyridin-3-yl)-1-oxo-2,3-dihydro-1H-indene-4-carboxamide (300 mg, 0.55 mmol) in trifluoroacetic acid (10 mL) was stirred at 50° C. overnight. After cooling to room temperature, the reaction mixture was adjusted to pH=8 with saturated aqueous sodium bicarbonate solution. The mixture was concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(6-methyl-5-(7-(methylamino)-1,6-naphthyridin-3-yl)pyridin-3-yl)-1-oxo-2,3-dihydro-1H-indene-4-carboxamide (12 mg, 0.03 mmol, 5%) as a yellow solid. ¹H NMR (400 MHz, DMSO-d₆) δ 10.66 (s, 1H), 9.00 (s, 1H), 8.85-8.84 (m, 2H), 8.31 (d, J=1.6 Hz. 1H), 8.17 (d, J=2.0 Hz, 1H), 8.09 (d, J=5.6 Hz, 1H), 7.83 (d, J=5.6 Hz, 1H), 7.61 (t, J=6.0 Hz, 1H), 6.95-6.94 (m, 1H), 6.63 (s, 1H), 3.37-3.34 (m, 2H), 2.87 (d, J=4.0 Hz, 3H), 2.69-2.66 (m, 2H), 2.48 (s, 3H). MS (ESI) m/z 424.2 [M+H]⁺

### Example 164. Synthesis of N-(4-(2-cyanopropan-2-yl)pyridin-2-yl)-6-(7-(methylamino)-1,6-naphthyridin-3-yl)pyridazine-4-carboxamide (Compound 213)

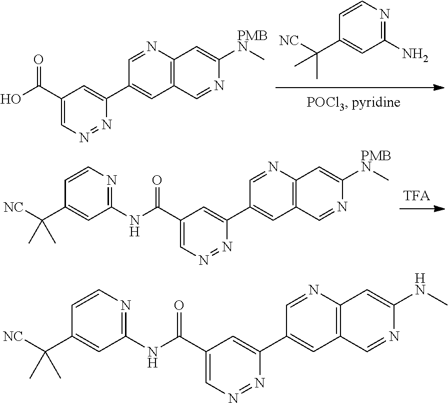

Step 1. A solution of 2-(2-aminopyridin-4-yl)-2-methylpropanenitrile (20 mg, 0.12 mmol), 6-(7-((4-methoxybenzyl)(methyl)amino)-1,6-naphthyridin-3-yl)pyridazine-4-carboxylic acid (50 mg, 0.12 mmol) and phosphoryl trichloride (93 mg, 0.6 mmol) in pyridine (3 mL) was stirred at room temperature for 4 hours. The mixture was diluted with water (50 mL) and extracted with ethyl acetate (50 mL×3). The combined organic layer was washed with brine (50 mL), dried over anhydrous sodium sulfate, filtered, and concentrated. The residue was purified by flash chromatography (silica, petroleum ether/ethyl acetate=2/1) to give N-(4-(2-cyanopropan-2-yl)pyridin-2-yl)-6-(7-((4-methoxybenzyl)(methyl)amino)-1,6-naphthyridin-3-yl)pyridazine-4-carboxamide (40 mg, 0.07 mmol, 61%) as a yellow solid. MS (ESI) m/z 545.2 [M+H]⁺

Step 2. A solution of N-(4-(2-cyanopropan-2-yl)pyridin-2-yl)-6-(7-((4-methoxybenzyl)(methyl)amino)-1,6-naphthyridin-3-yl)pyridazine-4-carboxamide (40 mg, 0.07 mmol) in trifluoroacetic acid (2 mL) was stirred at room temperature for 2 hours. The solvent was removed and the residue was purified by prep-HPLC (Column: Xbridge 21.2×250 mm C18, 10 μm, Mobile Phase A: water (10 mM ammonium bicarbonate), B: acetonitrile) to give N-(4-(2-cyanopropan-2-yl)pyridin-2-yl)-6-(7-(methylamino)-1,6-naphthyridin-3-yl)pyridazine-4-carboxamide (1.7 mg, 0.004 mmol, 6%) as a white solid. ¹H NMR (500 MHz, DMSO-d₆) δ 11.64 (s, 1H), 9.66 (d, J=2.3 Hz, 1H), 9.61 (d, J=2.0 Hz, 1H), 9.10 (s, 2H), 8.91 (d, J=2.0 Hz, 1H), 8.52 (dd, J=4.8, 3.6 Hz, 2H), 7.42 (dd, J=5.3, 1.8 Hz, 1H), 7.18 (d, J=5.0 Hz, 1H), 6.68 (s, 1H), 2.90 (d, J=4.9 Hz, 3H), 1.75 (s, 6H). MS (ESI) m/z 425.1 [M+H]⁺

### Example 165. Synthesis of 8-methyl-N-(4-methyl-3-(7-(methylamino)-1,6-naphthyridin-3-yl)phenyl)-5,6,7,8-tetrahydroimidazo[1,2-a]pyridine-3-carboxamide (Compound 214)

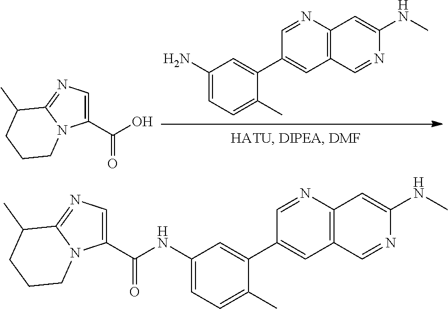

A mixture of 8-methyl-5,6,7,8-tetrahydroimidazo[1,2-a]pyridine-3-carboxylic acid (30 mg, 0.15 mmol), 3-(5-amino-2-methylphenyl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give 8-methyl-N-(4-methyl-3-(7-(methylamino)-1,6-naphthyridin-3-yl)phenyl)-5,6,7,8-tetrahydroimidazo[1,2-a]pyridine-3-carboxamide (20 mg, 0.047 mmol, 31%). ¹H NMR (400 MHz, DMSO-dr) δ 9.94 (s, 1H), 8.97 (d, J=14.2 Hz, 1H), 8.78 (dd, J=18.4, 2.3 Hz, 1H), 8.22 (d, J=2.0 Hz, 1H), 7.78 (s, 1H), 7.73-7.63 (m, 2H), 7.29 (t, J=11.9 Hz, 1H), 6.90 (d, J=5.0 Hz, 1H), 6.62 (s, 1H), 4.39 (d, J=13.5 Hz, 1H), 4.06 (t, J=9.7 Hz, 1H), 2.87 (d, J=4.9 Hz, 3H), 2.25 (d, J=12.0 Hz, 3H), 2.20-1.78 (m, 2H), 1.76-1.34 (m, 1H), 1.31 (d, J=6.9 Hz, 1H), 1.23 (s, 3H). MS (ESI) m/z 427 [M+H]⁺

### Example 166. Synthesis of 3,3-dimethyl-N-(4-methyl-3-(7-(methylamino)-1,6-naphthyridin-3-yl)phenyl)-2-oxoindoline-7-carboxamide (Compound 215)

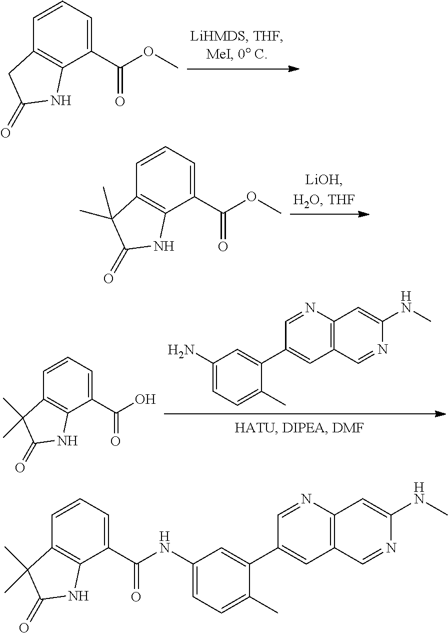

Step 1. Lithium bis(trimethylsilyl)amide (1 M, 8 mL, 8 mmol) was added to a solution of methyl 2-oxoindoline-7-carboxylate (400 mg, 2.09 mmol) and iodomethane (742 mg, 5.23 mmol) in tetrahydrofuran (20 mL) at 0° C. under argon. The mixture was stirred at 0° C. for 4 hours. The reaction mixture was quenched with saturated ammonium chloride aqueous solution (20 mL) and extracted with ethyl acetate (30 mL×2). The combined organic phases were concentrated and purified by flash chromatography (silica, petroleum ether/ethyl acetate=3/1) to give methyl 3,3-dimethyl-2-oxoindoline-7-carboxylate (400 mg, 1.83 mmol, 88%) as a colorless oil. MS (ESI) m/z 220 [M+H]⁺

Step 2. A mixture of methyl 3,3-dimethyl-2-oxoindoline-7-carboxylate (100 mg, 0.91 mmol) and lithium hydroxide (58 mg, 1.38 mmol) in tetrahydrofuran (5 mL) was stirred at room temperature for 1 hour. The reaction mixture was extracted with ethyl acetate (30 mL×2). The organic layers were discarded. The aqueous layer was adjusted pH=4 with hydrochloric acid (6 N) and extracted with ethyl acetate (30 mL×2). The combined organic phases were concentrated to give 3,3-dimethyl-2-oxoindoline-7-carboxylic acid (60 mg, 0.36 mmol, 75%) as a brown oil. MS (ESI) m/z 206 [M+H]⁺

Step 3. A mixture of 3,3-dimethyl-2-oxoindoline-7-carboxylic acid (30 mg, 0.15 mmol), 3-(5-amino-2-methylphenyl)-N-methyl-1,6-naphthyridin-7-amine (50 mg, 0.18 mmol), and N,N-diisopropylethylamine (67 mg, 0.54 mmol) in N,N-dimethylformamide (10 mL) was stirred at room temperature for 24 hours. The mixture was purified by prep-HPLC (Column: Xbridge 21.2×250 mm C18, 10 μm, Mobile Phase A: water (10 mM ammonium bicarbonate), B: acetonitrile) to give 3,3-dimethyl-N-(4-methyl-3-(7-(methylamino)-1,6-naphthyridin-3-yl)phenyl)-2-oxoindoline-7-carboxamide (20 mg, 0.068 mmol, 29%). ¹H NMR (400 MHz, DMSO-d₆) δ 10.30 (s, 1H), 10.06 (s, 1H), 8.99 (s, 1H), 8.81 (s, 1H), 8.23 (s, 1H), 7.78 (s, 3H), 7.51 (d, J=7.2 Hz, 1H), 7.35 (d, J=8.1 Hz, 1H), 7.12 (s, 1H), 6.91 (s, 1H), 6.63 (s, 1H), 2.86 (s, 3H), 2.50 (s, 3H), 2.29 (s, 3H), 1.29 (s, 6H). MS (ESI) m/z 452 [M+H]⁺

### Example 167. Synthesis of N-(4-methyl-3-(7-(methylamino)-1,6-naphthyridin-3-yl)phenyl)-5,6,7,8-tetrahydroimidazo[1,2-a]pyridine-3-carboxamide (Compound 216)

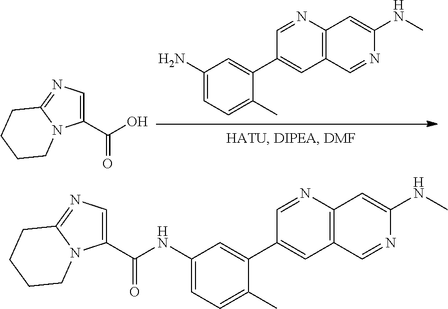 [SMILES: O=C(O)c1cnc2n1CCCC2 ; CNc1cc2ncc(-c3cc(N)ccc3C)cc2cn1 ; CNc1cc2ncc(-c3cc(NC(=O)c4cnc5n4CCCC5)ccc3C)cc2cn1]

A mixture of 5,6,7,8-tetrahydroimidazo[1,2-a]pyridine-3-carboxylic acid (30 mg, 0.15 mmol), 3-(5-amino-2-methylphenyl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 24 hours. The mixture was purified by prep-HPLC (Column: Xbridge 21.2×250 mm C18, 10 μm, Mobile Phase A: water (10 mM ammonium bicarbonate), B: acetonitrile) to give N-(4-methyl-3-(7-(methylamino)-1,6-naphthyridin-3-yl)phenyl)-5,6,7,8-tetrahydroimidazo[1,2-a]pyridine-3-carboxamide (30 mg, 0.068 mmol, 45%). ¹H NMR (400 MHz, DMSO-d₆) δ 9.93 (s, 3H), 8.98 (s, 1H), 8.80 (d, J=2.3 Hz, 1H), 8.22 (d, J=1.8 Hz, 3H), 7.77 (s, 1H), 7.73-7.63 (m, 2H), 7.31 (d, J=8.3 Hz, 1H), 6.89 (q, J=4.9 Hz, 1H), 6.62 (s, 1H), 4.24 (t, J=5.9 Hz, 2H), 2.89 (t, J=12.5 Hz, 3H), 2.79 (t, J=6.3 Hz, 2H), 2.26 (s, 3H), 2.00-1.70 (m, 4H). MS (ESI) m/z 413 [M+H]⁺

### Example 168. Synthesis of N-(2-fluoro-4-methyl-5-(7-(methylamino)-1,6-naphthyridin-3-yl)phenyl)-2-(1-fluorocyclopropyl)isonicotinamide (Compound 221)

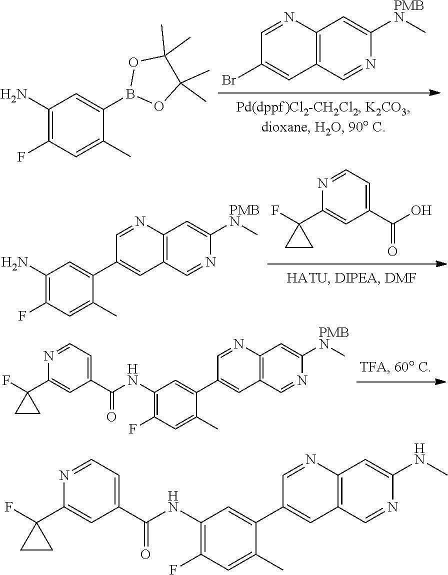

Step 1. A mixture of 2-fluoro-4-methyl-5-(4,4,5,5-tetramethyl-1,3,2-dioxaborolan-2-yl)aniline (530 mg, 2.11 mmol), 3-bromo-N-(4-methoxybenzyl)-N-methyl-1,6-naphthyridin-7-amine (678 mg, 1.90 mmol), potassium carbonate (690 mg, 5 mmol), and 1,1′-bis(diphenylphosphino)ferrocene-palladium(II)dichloride dichloromethane complex (171 mg, 0.21 mmol) in water (5 mL) and dioxane (20 mL) was stirred at 90° C. for 2 hours under nitrogen atmosphere. The mixture was filtered through celite. The filtrate was concentrated to give 3-(5-amino-4-fluoro-2-methylphenyl)-N-(4-methoxybenzyl)-N-methyl-1,6-naphthyridin-7-amine (600 mg, 71%) as a brown solid. MS (ESI) m/z 252 [M+H]⁺

Step 2. A mixture of 2-(1-fluorocyclopropyl)isonicotinic acid (30 mg, 0.15 mmol), 3-(5-amino-4-fluoro-2-methylphenyl)-N-(4-methoxybenzyl)-N-methyl-1,6-naphthyridin-7-amine (50 mg, 0.12 mmol), and N,N-diisopropylethylamine (67 mg, 0.54 mmol) in N,N-dimethylformamide (10 mL) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give N-(2-fluoro-5-(7-((4-methoxybenzyl)(methyl)amino)-1,6-naphthyridin-3-yl)-4-methylphenyl)-2-(1-fluorocyclopropyl)isonicotinamide (30 mg, 0.053 mmol, 44%) as a yellow solid. MS (ESI) m/z 403 [M+H]⁺

Step 3. A mixture of N-(2-fluoro-5-(7-((4-methoxybenzyl)(methyl)amino)-1,6-naphthyridin-3-yl)-4-methylphenyl)-2-(1-fluorocyclopropyl)isonicotinamide (35 mg, 0.10 mmol) and trifluoroacetic acid (5 mL) was stirred at 60° C. for 2 hours. After cooling to room temperature, the reaction mixture was concentrated. The residue was purified by prep-HPLC (Column: Xbridge 21.2×250 mm C18, 10 μm, Mobile Phase A: water (10 mM ammonium bicarbonate), B: acetonitrile) to give N-(2-fluoro-4-methyl-5-(7-(methylamino)-1,6-naphthyridin-3-yl)phenyl)-2-(1-fluorocyclopropyl)isonicotinamide (13 mg, 0.05 mmol, 50%) as a yellow solid. 1H NMR (400 MHz, DMSO-d₆) δ 10.59 (s, 1H), 8.97 (s, 1H), 8.81 (d, J=2.3 Hz, 1H), 8.72 (d, J=5.1 Hz, 1H), 8.25 (d, J=2.0 Hz, 1H), 8.10 (s, 1H), 7.78 (dd, J=5.0, 1.4 Hz, 1H), 7.59 (d, J=7.8 Hz, 1H), 7.37 (d, J=11.5 Hz, 1H), 6.93 (d, J=4.8 Hz, 1H), 6.62 (s, 1H), 2.87 (d, J=4.8 Hz, 3H), 2.32 (s, 3H), 1.67-1.34 (m, 2H), 1.23 (s, 2H). MS (ESI) m/z 446 [M+H]⁺

### Example 169. Synthesis of 4-(1-(dimethylamino)-2,2,2-trifluoroethyl)-N-(4-methyl-3-(7-(methylamino)-1,6-naphthyridin-3-yl)phenyl)picolinamide (Compound 218)

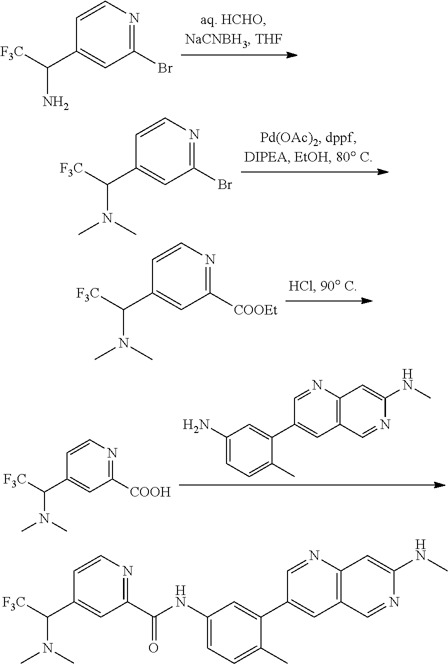

Step 1. A solution of formaldehyde (4.0 mL, 37% wt % in water) and 1-(2-bromopyridin-4-yl)-2,2,2-trifluoroethan-1-amine (400 mg, 1.57 mmol) in tetrahydrofuran (25 mL) was stirred at room temperature for 1 hour. Then sodium cyanoborohydride (293 mg, 4.71 mmol) was added at 0° C. The reaction solution was at room temperature for 12 hours. The reaction was quenched with hydrogen chloride (20 mL, 1 N), extracted with ethyl acetate (50 mL), washed with water (20 mL) and brine (20 mL), dried over sodium sulfate, filtered, and concentrated. The residue was purified by flash chromatography (silica, petroleum ether/ethyl acetated=1/1) to give 1-(2-bromopyridin-4-yl)-2,2,2-trifluoro-N,N-dimethylethan-1-amine (120 mg, 0.42 mmol, 23%) as a yellow solid. MS (ESI) m/z 285.0 [M+H]⁺

Step 2. A mixture of 1-(2-bromopyridin-4-yl)-2,2,2-trifluoro-N,N-dimethylethan-1-amine (120 mg, 0.42 mmol), palladium(II)acetate (9 mg, 0.0371 mmol), 1,1′-bis(diphenylphosphino)ferrocene (10 mg, 0.0185 mmol) and N,N-diisopropylethylamine (0.5 mL) in ethanol (20 mL) was stirred at 80° C. for 12 hours under the protection of carbon monoxide. After cooling to room temperature, the reaction mixture was concentrated. The residue was diluted with water (20 mL) and extracted with ethyl acetate (15 mL×3). The combined organic phases were washed brine (20 mL×2), dried over sodium sulfate, filtered, and concentrated. The residue was purified by flash chromatography (silica, petroleum ether/ethyl acetate=2/1) to give ethyl 4-(1-(dimethylamino)-2,2,2-trifluoroethyl)picolinate (50 mg, 0.18 mmol, 43%) as a yellow solid. MS (ESI) m/z 277.1 [M+H]⁺

Step 3. A solution of ethyl 4-(1-(dimethylamino)-2,2,2-trifluoroethyl)picolinate (50 mg, 0.18 mmol) in 6 N aqueous hydrochloride (5 mL) was stirred at 90° C. for 12 hours. The reaction mixture was concentrated to give 4-(1-(dimethylamino)-2,2,2-trifluoroethyl)picolinic acid (20 mg, 0.081 mmol, 45%) as a yellow solid, which was used without further purification. MS (ESI) m/z 249.1 [M+H]⁺

Step 4. A solution of 4-(1-(dimethylamino)-2,2,2-trifluoroethyl)picolinic acid (20 mg, 0.081 mmol), 3-(5-amino-2-methylphenyl)-N-methyl-1,6-naphthyridin-7-amine (21 mg, 0.081 mmol), 1-[bis(dimethylamino)methylene]-1H-1,2,3-triazolo[4,5-b]pyridinium 3-oxid hexafluorophosphate (62 mg, 0.16 mmol) and N,N-diisopropylethylamine (0.1 mL) in N,N-dimethylformamide (3 mL) was stirred at room temperature for 12 hours. The reaction was diluted with ethyl acetate (50 mL), washed with water (20 mL) and brine (20 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(1-(dimethylamino)-2,2,2-trifluoroethyl)-N-(4-methyl-3-(7-(methylamino)-1,6-naphthyridin-3-yl)phenyl)picolinamide (6.2 mg, 0.013 mmol, 16%) as a yellow solid. ¹H NMR (400 MHz, DMSO-d₆) δ 10.76 (s, 1H), 9.00 (s, 1H), 8.85-8.81 (m, 2H), 8.30 (s, 1H), 8.18 (s, 1H), 7.95 (s, 1H), 7.94-7.87 (m, 1H), 7.69-7.67 (m, 1H), 7.37 (d, J=8.8 Hz, 1H), 6.99 (s, 1H), 6.63 (s, 1H), 4.94-4.87 (m, 1H), 3.06 (d, J=4.0 Hz, 3H), 2.42 (s, 9H). MS (ESI) m/z 495.1 [M+H]⁺

### Example 170. Synthesis of 1-hydroxy-N-(6-methyl-5-(7-(methylamino)-1,6-naphthyridin-3-yl)pyridin-3-yl)-2,3-dihydro-1H-indene-4-carboxamide (Compound 219)

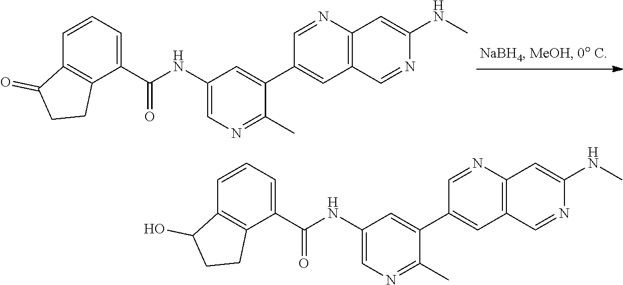

To a solution of N-(6-methyl-5-(7-(methylamino)-1,6-naphthyridin-3-yl)pyridin-3-yl)-1-oxo-2,3-dihydro-1H-indene-4-carboxamide (140 mg, 0.33 mmol) in methanol (5 mL) was added sodium borohydride (12.5 mg, 0.33 mmol) at 0° C. The mixture was stirred at 0° C. for 2 hours, and then quenched with a solution of ammonium chloride (5 mL). The mixture was concentrated under reduced pressure. The residue was purified by prep-HPLC (Column: Welch Xtimate 21.2×250 mm C18, 10 μm, Mobile Phase: A: water (10 mM ammonium bicarbonate), B: acetonitrile) to give 1-hydroxy-N-(6-methyl-5-(7-(methylamino)-1,6-naphthyridin-3-yl)pyridin-3-yl)-2,3-dihydro-1H-indene-4-carboxamide (2.6 mg, 0.006 mmol, 2%) as a yellow solid. ¹H NMR (400 MHz, DMSO-d₆) δ 10.42 (s, 1H), 8.99 (s, 1H), 8.85-8.82 (m, 2H), 8.30 (d, J=2.0 Hz, 1H), 8.16 (d, J=2.4 Hz, 1H), 7.61 (d, J=7.6 Hz, 1H), 7.51 (d, J=7.6 Hz, 1H), 7.38-7.35 (m, 1H), 6.95-6.94 (m, 1H), 6.63 (s, 1H), 5.35 (d, J=6.0 Hz, 1H), 5.09-5.07 (m, 1H), 3.16-3.13 (m, 1H), 2.94-2.90 (m, 1H), 2.86 (d, J=5.2 Hz, 3H), 2.51 (s, 3H), 2.36-2.32 (m, 1H), 1.80-1.77 (m, 1H). MS (ESI) m/z 426.1 [M+H]⁺

### Example 171. Synthesis of N-(4-methyl-3-(7-(methylamino)-1,6-naphthyridin-3-yl)phenyl)-6-(trifluoromethyl)-5,6,7,8-tetrahydroimidazo[1,2-a]pyridine-3-carboxamide (Compound

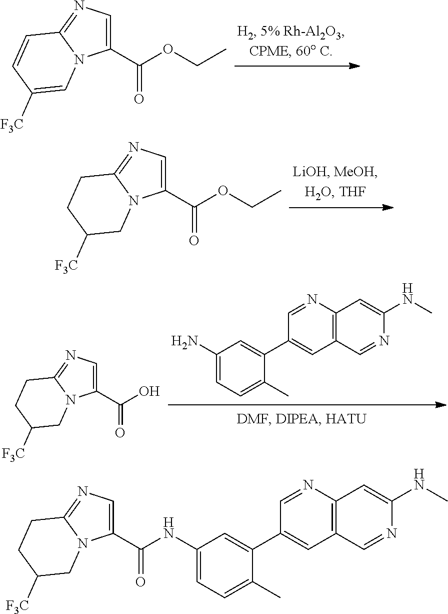 [SMILES: CCOC(=O)c1cnc2ccc(C(F)(F)F)cn12 ; CCOC(=O)c1cnc2n1CC(C(F)(F)F)CC2 ; O=C(O)c1cnc2n1CC(C(F)(F)F)CC2 ; CNc1cc2ncc(-c3cc(N)ccc3C)cc2cn1 ; CNc1cc2ncc(-c3cc(NC(=O)c4cnc5n4CC(C(F)(F)F)CC5)ccc3C)cc2cn1]

Step 1. To a solution of ethyl 6-(trifluoromethyl)imidazo[1,2-a]pyridine-3-carboxylate (200 mg, 0.775 mmol) in cyclopentyl methyl ether (5 mL) at 60° C. was added rhodium-aluminum oxide (5%, 200 mg). The reaction mixture was stirred at the same temperature for 16 hours under hydrogen atmosphere. After cooling to room temperature, the reaction was filtered through celite. The filtrate was concentrated in vacuo. The residue was purified by flash chromatography (petroleum ether/ethyl acetate=1/1) to afford ethyl 6-(trifluoromethyl)-5,6,7,8-tetrahydroimidazo[1,2-a]pyridine-3-carboxylate (180 mg, 0.69 mmol, 90%) as a white solid. MS (ESI) m/z 263.2 [M+H]⁺

Step 2. Lithium hydroxide monohydrate (87 mg, 2.06 mmol) was added to a solution of ethyl 6-(trifluoromethyl)-5,6,7,8-tetrahydroimidazo[1,2-a]pyridine-3-carboxylate (180 mg, 0.69 mmol) in tetrahydrofuran (3 mL), methanol (3 mL), and water (3 mL) at 0° C. The reaction mixture was stirred at 25° C. for 3 hours. The reaction solution was diluted with water (20 mL) and concentrated to remove tetrahydrofuran. The aqueous layer was acidified to pH=3 with 4 N HCl and purified by prep-HPLC to give 6-(trifluoromethyl)-5,6,7,8-tetrahydroimidazo[1,2-a]pyridine-3-carboxylic acid (100 mg, 0.43 mmol, 63%) as a white solid. MS (ESI) m/z 235.2 [M+H]⁺

Step 3. To a solution of 6-(trifluoromethyl)-5,6,7,8-tetrahydroimidazo[1,2-a]pyridine-3-carboxylic acid (50 mg, 0.21 mmol) in N,N-dimethylformamide (4 mL) was added N,N-diisopropylethylamine (82 mg, 0.64 mmol), 1-[bis(dimethylamino)methylene]-1H-1,2,3-triazolo[4,5-b]pyridinium 3-oxid hexafluorophosphate (121 mg, 0.32 mmol) and 3-(5-amino-2-methylphenyl)-N-methyl-1,6-naphthyridin-7-amine (56 mg, 0.21 mmol). The mixture was stirred at room temperature for 1 hour. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(4-methyl-3-(7-(methylamino)-1,6-naphthyridin-3-yl)phenyl)-6-(trifluoromethyl)-5,6,7,8-tetrahydroimidazo[1,2-a]pyridine-3-carboxamide (2.3 mg, 0.005 mmol) as a yellow solid. ¹H NMR (500 MHz, DMSO-d₆) δ 1.86-1.94 (m, 1H), 2.15-2.17 (m, 1H), 2.26 (s, 3H), 2.86 (d, J=5.0 Hz, 3H), 2.88-2.98 (m, 2H), 3.19 (s, 1H), 4.08-4.13 (m, 1H), 4.72 (dd, J₁=5.5 Hz, J₂=13.25 Hz, 1H), 6.62 (s, 1H), 6.88 (q, J=5.0 Hz, 1H), 7.31 (d, J=8.0 Hz, 1H), 7.65-7.70 (m, 2H), 7.82 (s, 1H), 8.21 (d, J=2.0 Hz, 1H), 8.80 (d, J=2.0 Hz, 1H), 8.98 (s, 1H), 10.00 (s, 1H). MS (ESI) m/z 481.1 [M+H]⁺

### Example 172. Synthesis of 2-(2-cyanopropan-2-yl)-N-(2-fluoro-4-methyl-5-(7-(methylamino)-1,6-naphthyridin-3-yl)phenyl)isonicotinamide (Compound 217)

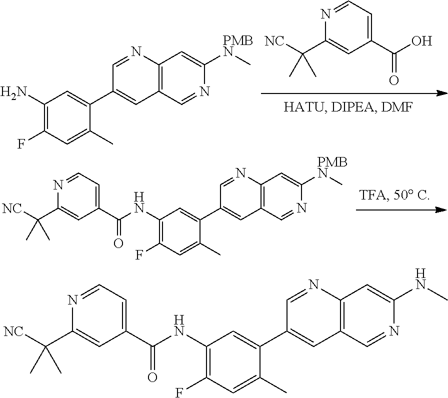

Step 1. To a solution of 3-(5-amino-4-fluoro-2-methylphenyl)-N-(4-methoxybenzyl)-N-methyl-1,6-naphthyridin-7-amine (55 mg, 0.14 mmol) in N,N-dimethylformamide (3 mL) was added N,N-diisopropylethylamine (52.9 mg, 0.4 mmol), 1-[bis(dimethylamino)methylene]-1H-1,2,3-triazolo[4,5-b]pyridinium 3-oxid hexafluorophosphate (77.9 mg, 0.21 mmol) and 2-(2-cyanopropan-2-yl)isonicotinic acid (32.5 mg, 0.17 mmol). The mixture was stirred at room temperature for 2 hours. The reaction was diluted with ethyl acetate (100 mL×3), washed with saturated sodium bicarbonate aqueous solution (50 mL) and brine (40 mL), dried over sodium sulfate, filtered, and concentrated. The residue was purified by flash chromatography (petroleum ether/ethyl acetate=1/1) to give 2-(2-cyanopropan-2-yl)-N-(2-fluoro-5-(7-((4-methoxybenzyl)(methyl)amino)-1,6-naphthyridin-3-yl)-4-methylphenyl)isonicotinamide (60 mg, 0.1 mmol, 62%) as a yellow solid. MS (ESI) m/z 475.1 [M+H]⁺

Step 2. A solution of 2-(2-cyanopropan-2-yl)-N-(2-fluoro-5-(7-((4-methoxybenzyl)(methyl)amino)-1,6-naphthyridin-3-yl)-4-methylphenyl)isonicotinamide (60 mg, 0.1 mmol) in trifluoroacetic acid (3 mL) was stirred at 50° C. for 2 hours. The reaction solution was concentrated. The residue was dissolved in ethyl acetate (40 mL×3), washed with saturated sodium bicarbonate aqueous solution (50 mL) and brine (40 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 2-(2-cyanopropan-2-yl)-N-(2-fluoro-4-methyl-5-(7-(methylamino)-1,6-naphthyridin-3-yl)phenyl)isonicotinamide (31.5 mg, 0.069 mmol, 69%) as a yellow solid. ¹H NMR (400 MHz, DMSO-d₆) δ 10.47 (s, 1H), 8.99 (s, 1H), 8.82 (d, J=4.5 Hz, 2H), 8.27 (s, 2H), 8.00 (d, J=7.9 Hz, 1H), 7.88 (dd, J=5.3, 2.0 Hz, 1H), 7.39 (d, J=11.7 Hz, 1H), 6.97 (s, 1H), 6.62 (s, 1H), 2.87 (d, J=3.8 Hz, 3H), 2.32 (s, 3H), 1.76 (s, 6H). MS (ESI) m/z 455.1 [M+H]⁺

### Example 173. Synthesis of N-(2-fluoro-4-methyl-5-(7-(methylamino)-1,6-naphthyridin-3-yl)phenyl)-2-(2-fluoropropan-2-yl)isonicotinamide (Compound 222)

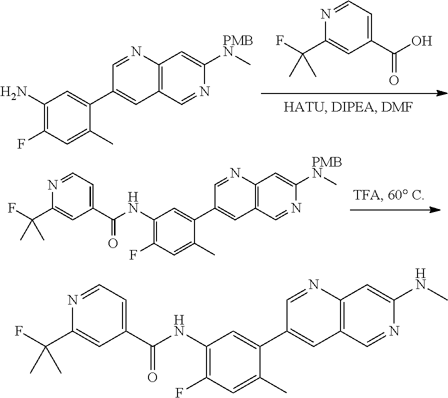

Step 1. A mixture of 2-(2-fluoropropan-2-yl)isonicotinic acid (30 mg, 0.15 mmol), 3-(5-amino-4-fluoro-2-methylphenyl)-N-(4-methoxybenzyl)-N-methyl-1,6-naphthyridin-7-amine (50 mg, 0.12 mmol), and N,N-diisopropylethylamine (67 mg, 0.54 mmol) in N,N-dimethylformamide (10 mL) was stirred at room temperature for 16 hours. The mixture was purified by prep-HPLC (Column: Xbridge 21.2×250 mm C18, 10 μm, Mobile Phase A: water (10 mM ammonium bicarbonate), B: acetonitrile) to give N-(2-fluoro-5-(7-((4-methoxybenzyl)(methyl)amino)-1,6-naphthyridin-3-yl)-4-methylphenyl)-2-(2-fluoropropan-2-yl)isonicotinamide (30 mg, 0.053 mmol, 44%). MS (ESI) m/z 568 [M+H]⁺

Step 2. A mixture of N-(2-fluoro-5-(7-((4-methoxybenzyl)(methyl)amino)-1,6-naphthyridin-3-yl)-4-methylphenyl)-2-(2-fluoropropan-2-yl)isonicotinamide (35 mg, 0.10 mmol) and trifluoroacetic acid (5 mL) was stirred at 60° C. for 2 hours. After cooling to room temperature, the reaction mixture was concentrated. The residue was purified by prep-HPLC (Column: Xbridge 21.2×250 mm C18, 10 μm, Mobile Phase A: water (10 mM ammonium bicarbonate), B: acetonitrile) to give N-(2-fluoro-4-methyl-5-(7-(methylamino)-1,6-naphthyridin-3-yl)phenyl)-2-(2-fluoropropan-2-yl)isonicotinamide (13 mg, 0.05 mmol, 50%). ¹H NMR (400 MHz, DMSO-d₆) δ 10.57 (s, 1H), 8.97 (s, 1H), 8.78 (dd, J=12.8, 3.7 Hz, 2H), 8.23 (d, J=1.9 Hz, 1H), 8.05 (s, 1H), 7.83 (d, J=5.0 Hz, 1H), 7.58 (d, J=7.8 Hz, 1H), 7.37 (d, J=11.5 Hz, 1H), 6.91 (q, J=4.8 Hz, 1H), 6.62 (s, 1H), 2.86 (d, J=4.9 Hz, 3H), 2.32 (s, 3H), 1.74 (s, 3H), 1.68 (s, 3H). MS (ESI) m/z 448 [M+H]⁺

### Example 174. Synthesis of 3-(2-cyanopropan-2-yl)-N-(2-fluoro-4-methyl-5-(7-(methylamino)-1,6-naphthyridin-3-yl)phenyl)benzamide (Compound 223)

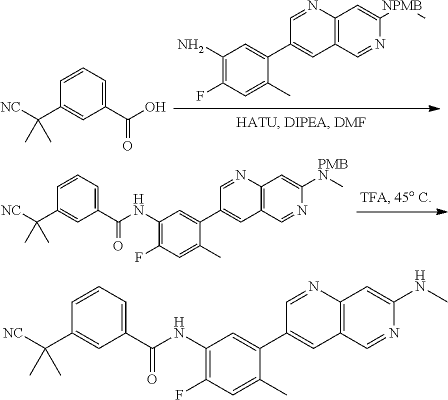

Step 1. To a solution of 3-(2-cyanopropan-2-yl)benzoic acid (50 mg, 0.26 mmol) in N,N-dimethylformamide (4 mL) was added N,N-diisopropylethylamine (102 mg, 0.79 mmol), 1-[bis(dimethylamino)methylene]-1H-1,2,3-triazolo[4,5-b]pyridinium 3-oxid hexafluorophosphate (151 mg, 0.40 mmol) and 3-(5-amino-4-fluoro-2-methylphenyl)-N-(4-methoxybenzyl)-N-methyl-1,6-naphthyridin-7-amine (106 mg, 0.26 mmol). The mixture was stirred at room temperature for 1 hour. The reaction was diluted with ethyl acetate (100 mL), washed with saturated sodium bicarbonate aqueous solution (50 mL) and brine (40 mL), dried over sodium sulfate, filtered, and concentrated. The residue was purified by flash chromatography (petroleum ether/ethyl acetate=1/1) to give 3-(2-cyanopropan-2-yl)-N-(2-fluoro-5-(7-((4-methoxybenzyl(methyl)amino)-1,6-naphthyridin-3-yl)-4-methylphenyl)benzamide (80 mg, 0.14 mmol, 53%) as a yellow solid. MS (ESI) m/z 574.3 [M+H]⁺

Step 2. A mixture of 3-(2-cyanopropan-2-yl)-N-(2-fluoro-5-(7-((4-methoxybenzyl)(methyl)amino)-1,6-naphthyridin-3-yl)-4-methylphenyl)benzamide (80 mg, 0.14 mmol) in trifluoroacetic acid (4 mL) was stirred at 45° C. for 1 hour. The reaction was concentrated and the residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 3-(2-cyanopropan-2-yl)-N-(2-fluoro-4-methyl-5-(7-(methylamino)-1,6-naphthyridin-3-yl)phenyl)benzamide (10 mg, 0.022 mmol, 17%) as a yellow solid. ¹H NMR (400 MHz, DMSO-d₆) δ 1.74 (s, 6H), 2.32 (s, 3H), 2.86 (d, J=4.4 Hz, 3H), 6.61 (s, 1H), 6.92 (d, J=4.8 Hz, 1H), 7.35 (d, J=11.2 Hz, 1H), 7.55-7.62 (m, 2H), 7.76 (d, J=8.0 Hz, 1H), 7.96 (d, J=8.0 Hz, 1H), 8.10 (s, 1H), 8.24 (d, J=1.6 Hz, 1H), 8.80 (d, J=2.0 Hz, 1H), 8.97 (s, 1H), 10.28 (s, 1H). MS (ESI) m/z 454.2 [M+H]⁺

### Example 175. Synthesis of N-(2-fluoro-4-methyl-5-(7-(methylamino)-1,6-naphthyridin-3-yl)phenyl)-4-(trifluoromethyl)picolinamide (Compound 224)

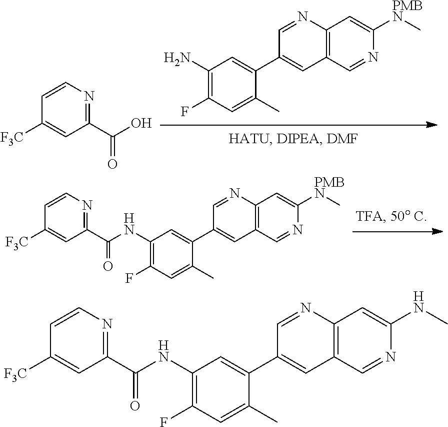

Step 1. A mixture of 4-(trifluoromethyl)picolinic acid (40 mg, 0.21 mmol), 3-(5-amino-4-fluoro-2-methylphenyl)-N-(4-methoxybenzyl)-N-methyl-1,6-naphthyridin-7-amine (84 mg, 0.21 mmol), I-[bis(dimethylamino)methylene]-1H-1,2,3-triazolo[4,5-b]pyridinium 3-oxid hexafluorophosphate (160 mg, 0.42 mmol), and N,N-diisopropylethylamine (82 mg, 0.63 mmol) in N,N-dimethylformamide (1 mL) was stirred at room temperature for 1 hour. The mixture was purified by prep-HPLC (Column: Xbridge 21.2×250 mm C18, 10 μm, Mobile Phase A: water (10 mM ammonium bicarbonate), B: acetonitrile) to give N-(2-fluoro-5-(7-((4-methoxybenzyl)(methyl)amino)-1,6-naphthyridin-3-yl)-4-methylphenyl)-4-(trifluoromethyl)picolinamide (60 mg, 0.10 mmol, 50%). MS (ESI) m/z 576.1 [M+H]⁺

Step 2. A mixture of N-(2-fluoro-5-(7-((4-methoxybenzyl)(methyl)amino)-1,6-naphthyridin-3-yl)-4-methylphenyl)-4-(trifluoromethyl)picolinamide (60 mg, 0.10 mmol) and trifluoroacetic acid (5 ml) was stirred at 50° C. overnight. The mixture was purified by prep-HPLC (Column: Xbridge 21.2×250 mm C18, 10 μm, Mobile Phase A: water (10 mM ammonium bicarbonate), B: acetonitrile) to give N-(2-fluoro-4-methyl-5-(7-(methylamino)-1,6-naphthyridin-3-yl)phenyl)-4-(trifluoromethyl)picolinamide (42 mg, 0.09 mmol, 92%). ¹H NMR (400 MHz, DMSO-d₆) δ 10.55 (s, 1H), 9.05 (d, J=4.8 Hz, 1H), 8.97 (s, 1H), 8.80 (s, 1H), 8.34 (s, 1H), 8.23 (d, J=1.2 Hz, 1H), 8.13 (d, J=4.0 Hz, 1H), 7.92 (d, J=8.0 Hz, 1H), 7.38 (d, J=11.6 Hz, 1H), 6.92-6.91 (m, 1H), 6.62 (s, 1H), 2.87 (d, J=4.8 Hz, 3H), 2.31 (s, 3H). MS (ESI) m/z 456.0 [M+H]⁺

### Example 176. Synthesis of 6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide (Compound 225)

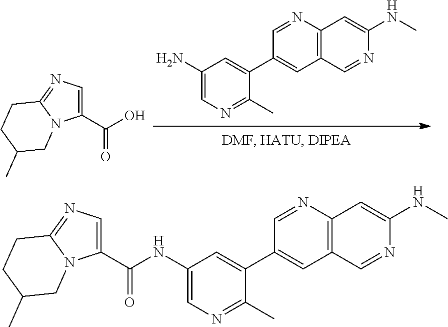 [SMILES: CC1CCc2ncc(C(=O)O)n2C1 ; CNc1cc2ncc(-c3cc(N)cnc3C)cc2cn1 ; CNc1cc2ncc(-c3cc(NC(=O)c4cnc5n4CC(C)CC5)cnc3C)cc2cn1]

To a solution of 6-methyl-5,6,7,8-tetrahydroimidazo[1,2-a]pyridine-3-carboxylic acid (50 mg, 0.28 mmol) in N,N-dimethylformamide (4 mL) was added N,N-diisopropylethylamine (108 mg, 0.83 mmol), 1-[bis(dimethylamino)methylene]-1H-1,2,3-triazolo[4,5-b]pyridinium 3-oxid hexafluorophosphate (158 mg, 0.42 mmol) and 3-(5-amino-2-methylpyridin-3-yl)-N-methyl-1,6-naphthyridin-7-amine (74 mg, 0.28 mmol). The mixture was stirred at room temperature for 1 hour. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide (2.4 mg, 0.005 mmol) as a yellow solid. ¹H NMR (500 MHz, DMSO-d₆) δ 1.04 (d, J=6.0 Hz, 3H), 1.46-1.54 (m, 1H), 1.89 (s, 1H), 2.03 (s, 1H), 2.46 (s, 3H), 2.77-2.80 (m, 1H), 2.87 (d, J=4.5 Hz, 4H), 3.61 (t, J=10.5 Hz, 1H), 4.52 (dd, J₁=4.5 Hz, J₂=13.75 Hz, 1H), 6.63 (s, 1H), 6.94 (d, J=5.0 Hz, 1H), 7.81 (s, 1H), 8.10 (s, 1H), 8.29 (s, 1H), 8.82 (dd, J₁=2.0 Hz, J₂=19.75 Hz, 2H), 8.99 (s, 1H), 10.13 (s, 1H). MS (ESI) m/z 428.1 [M+H]⁺

### Example 177. Synthesis of 2-((1,1-difluoropropan-2-yl)amino)-N-(6-methyl-5-(7-(methylamino)-1,6-naphthyridin-3-yl)pyridin-3-yl)benzamide (Compound 226)

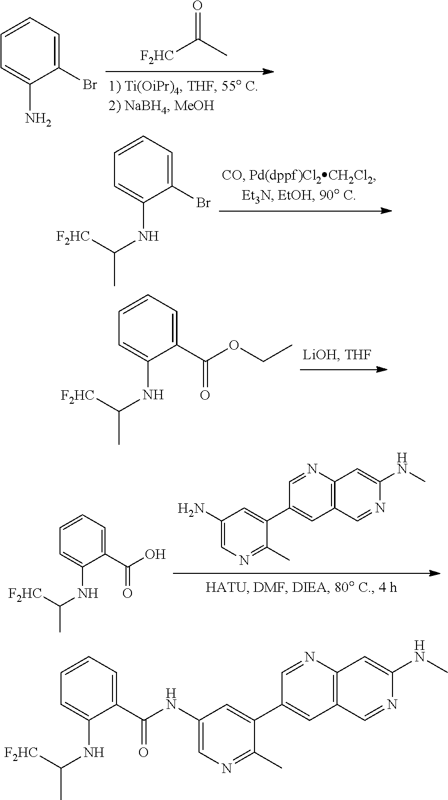

Step 1. A mixture of 2-bromoaniline (1.72 g, 10 mmol), 1,1-difluoropropan-2-one (1.88 g, 20 mmol), and titanium(IV) isopropoxide (1.5 mL) in tetrahydrofuran (6 mL) was stirred at 55° C. under nitrogen atmosphere for 16 hours. The mixture was cooled and sodium borohydride (760 mg, 20 mmol) in methanol (10 mL) was added. The mixture was stirred at room temperature for another 1 hour. The mixture was poured into water (50 mL) and extracted with ethyl acetate (50 mL×2). The combined organic phase was concentrated. The residual was purified by flash chromatography (silica, 20% ethyl acetate in petroleum ether) to afford 2-bromo-N-(1,1-difluoropropan-2-yl)aniline (1.6 g, 6.39 mmol, 64%) as a colorless oil. MS (ESI) m/z 250 [M+H]⁺

Step 2. A solution of 2-bromo-N-(1,1-difluoropropan-2-yl)aniline (1.6 g, 6.39 mmol), 1,1′-bis(diphenylphosphino)ferrocene-palladium(II)dichloride dichloromethane complex (522 mg, 0.64 mmol) and triethylamine (2.6 mL, 19.17 mmol) in ethanol (30 mL) was stirred at 90° C. for 16 hours under carbon monoxide atmosphere. The reaction was cooled to room temperature and concentrated. The residue was quenched with water (30 mL) and extracted with ethyl acetate (30 mL×2). The combined organic phases were concentrated and purified by flash chromatography (silica, petroleum ether/ethyl acetate=1/1) to give ethyl 2-((1,1-difluoropropan-2-yl)amino)benzoate (1.02 g, 4.19 mmol, 66%) as a white solid. MS (ESI) m/z 244 [M+H]⁺

Step 3. A mixture of ethyl 2-((1,1-difluoropropan-2-yl)amino)benzoate (300 mg, 1.23 mmol) and lithium hydroxide (234 mg, 6.15 mmol) in tetrahydrofuran (10 mL) was stirred at room temperature for 16 hours. The reaction mixture was extracted with ethyl acetate (30 mL×2). The organic layers were discarded. The aqueous layer was adjusted to pH=4 with hydrochloric acid (2 N) and extracted with ethyl acetate (30 mL×2). The combined organic phases were concentrated to give 2-((1,1-difluoropropan-2-yl)amino)benzoic acid (180 mg, 0.84 mmol, 68%) as a white solid. MS (EST) m/z 216 [M+H]⁺

Step 4. A mixture of 2-((1,1-difluoropropan-2-yl)amino)benzoic acid (50 mg, 0.23 mmol), 3-(5-amino-2-methylphenyl)-N-methyl-1,6-naphthyridin-7-amine (61 mg, 0.23 mmol), 1-[bis(dimethylamino)methylene]-1H-1,2,3-triazolo[4,5-b]pyridinium 3-oxid hexafluorophosphate (175 mg, 0.46 mmol) and N,N-diisopropylethylamine (89 mg, 0.69 mmol) in N,N-dimethylformamide (3 mL) was stirred at 80° C. for 4 hours. The reaction mixture was purified by prep-HPLC to give 2-((1,1-difluoropropan-2-yl)amino)-N-(6-methyl-5-(7-(methylamino)-1,6-naphthyridin-3-yl)pyridin-3-yl)benzamide (15 mg, 0.032 mmol, 14%) as a yellow solid. ¹H NMR (400 MHz, DMSO-d₆) δ 10.41 (s, 1H), 9.04 (s, 1H), 8.90 (d, 1=2.1 Hz, 1H), 8.82 (d, J=2.3 Hz, 1H), 8.42 (s, 1H), 8.18 (s, 1H), 7.80 (d, J=7.7 Hz, 1H), 7.67 (d, J=8.9 Hz, 1H), 7.38 (t, J=7.7 Hz, 1H), 7.13 (s, 1H), 6.96 (d, J=8.5 Hz, 1H), 6.73 (t, J=7.5 Hz, 1H), 6.63 (s, 1H), 6.24-5.92 (m, 1H), 4.13 (s, 1H), 2.89 (s, 3H), 2.51 (s, 3H), 1.20 (d, J=6.6 Hz, 3H). MS (ESI) m/z 463 [M+H]⁺

### Example 178. Synthesis of 4-(sec-butyl)-N-(6-methyl-5-(7-(methylamino)-1,6-naphthyridin-3-yl)pyridin-3-yl)picolinamide (Compound 227)

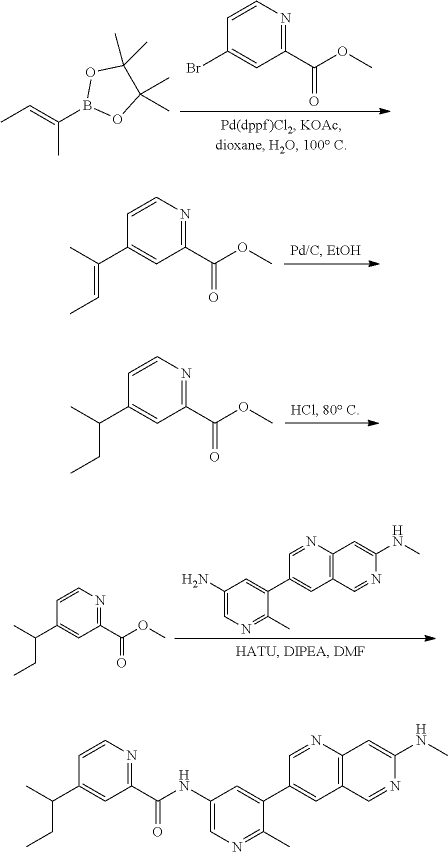

Step 1. A mixture of 2-(but-2-en-2-yl)-4,4,5,5-tetramethyl-1,3,2-dioxaborolane (500 mg, 2.75 mmol), methyl 4-bromopicolinate (594 mg, 2.75 mmol), [1,1′-bis(diphenylphosphino)ferrocene]dichloropalladium(II) (31.3 mg, 0.037 mmol) and potassium acetate (725 mg, 7.40 mmol) in 1,4-dioxane/water (10 mL/0.5 mL) was stirred at 100° C. for 8 hours under nitrogen atmosphere. The mixture was cooled to room temperature and diluted with water (10 mL). The mixture was extracted with ethyl acetate (10 mL×3). The organic phases were dried over anhydrous sodium sulfate and filtered. The filtrate was concentrated. The residue was purified by flash chromatography (silica, petroleum ether/ethyl acetate=1/2) to afford methyl 4-(but-2-en-2-yl)picolinate (50 mg, 0.26 mmol, 10%) as a yellow oil. MS (EST) m/z 192.1 [M+H]⁺

Step 2. A mixture of methyl 4-(but-2-en-2-yl)picolinate (50 mg, 0.26 mmol) and palladium (100% on activated carbon, 20 mg) in ethanol (10 mL) was stirred at 25° C. for 12 hours under hydrogen atmosphere. The mixture was filtered and the filtrate was concentrated to give methyl 4-(sec-butyl)picolinate (25 mg, 0.13 mmol, 50%) as a yellow solid. MS (ESI) m/z 194.1 [M+H]⁺

Step 3. A solution of methyl 4-(sec-butyl)picolinate (25 mg, 0.13 mmol) in 6 N aqueous hydrochloride (5 mL) was stirred at 80° C. for 8 hours. The reaction mixture was concentrated to give 4-(sec-butyl)picolinic acid (18 mg, 0.10 mmol, 77%) as a yellow solid, which was used without further purification. MS (ESI) m/z 180.0 [M+H]⁺

Step 4. To an N,N-dimethylformamide (5 mL) solution of 4-(sec-butyl)picolinic acid (18 mg, 0.10 mmol) and 1-[bis(dimethylamino)methylene]-1H-1,2,3-triazolo[4,5-b]pyridinium 3-oxid hexafluorophosphate (75 mg, 0.20 mmol) was added 3-(5-amino-2-methylpyridin-3-yl)-N-methyl-1,6-naphthyridin-7-amine (26.5 mg, 0.10 mmol) and N,N-diisopropylethylamine (0.16 mL). The mixture was stirred at room temperature for 1 hour. The mixture was diluted with water (10 mL) and extracted with ethyl acetate (10 mL×3). The combined organic layer was washed with brine (10 mL), dried over anhydrous sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(sec-butyl)-N-(6-methyl-5-(7-(methylamino)-1,6-naphthyridin-3-yl)pyridin-3-yl)picolinamide (5 mg, 0.012 mmol, 12%) as a yellow solid. ¹H NMR (400 MHz, DMSO-d₆) (510.93 (s, 1H), 9.05 (d, J=2.0 Hz, 1H), 8.99 (s, 1H), 8.87 (d, J=2.0 Hz, 1H), 8.66 (d, J=4.4 Hz, 1H), 8.32-8.31 (m, 2H), 8.01 (s, 1H), 7.57-7.55 (m, 1H), 6.96 (s, 1H), 6.64 (s, 1H), 2.88 (d, J=4.8 Hz, 3H), 2.81-2.79 (m, 1H), 2.51 (s, 3H), 1.65-1.61 (m, 2H), 1.25 (d, J=7.2 Hz, 3H), 0.80 (q, J=7.2 Hz, 3H). MS (ESI) m/z 427.1 [M+H]⁺

### Example 179. Synthesis of N-(4-methyl-3-(7-(methylamino)-1,6-naphthyridin-3-yl)phenyl)-4,5,6,7-tetrahydrobenzo[d]isoxazole-3-carboxamide (Compound 228)

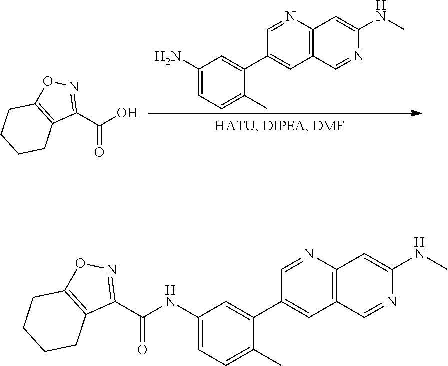 [SMILES: O=C(O)c1noc2c1CCCC2 ; CNc1cc2ncc(-c3cc(N)ccc3C)cc2cn1 ; CNc1cc2ncc(-c3cc(NC(=O)c4noc5c4CCCC5)ccc3C)cc2cn1]

A solution of 4,5,6,7-tetrahydrobenzo[d]isoxazole-3-carboxylic acid (30 mg, 0.18 mmol), 3-(5-amino-2-methylphenyl)-N-methyl-1,6-naphthyridin-7-amine (37.8 mg, 0.14 mmol), 1-[bis(dimethylamino)methylene]-1H-1,2,3-triazolo[4,5-b]pyridinium 3-oxid hexafluorophosphate (102.6 mg, 0.27 mmol) and N,N-diisopropylethylamine (69.7 mg, 0.54 mmol) in N,N-dimethylformamide (3 mL) was stirred at room temperature for 2 hours. The residue was purified by prep-HPLC (Column: Xbridge 21.2×250 mm C18, 10 μm, Mobile Phase A: water (10 mM ammonium bicarbonate), B: acetonitrile) to give N-(4-methyl-3-(7-(methylamino)-1,6-naphthyridin-3-yl)phenyl)-4,5,6,7-tetrahydrobenzo[d]isoxazole-3-carboxamide (35.2 mg, 0.085 mmol, 47%) as a yellow solid. 1H NMR (400 MHz, DMSO-d₆) δ 10.66 (s, 1H), 8.98 (s, 1H), 8.80 (d, J=2.2 Hz, 1H), 8.22 (d, J=1.6 Hz, 1H), 7.78 (d, J=26.7 Hz, 1H), 7.73 (d, J=8.3 Hz, 1H), 7.33 (d, J=8.3 Hz, 1H), 6.90 (d, J=5.0 Hz, 1H), 6.62 (s, 1H), 2.87 (d, J=4.9 Hz, 3H), 2.76 (t, J=5.8 Hz, 2H), 2.59 (t, J=5.7 Hz, 2H), 2.27 (s, 3H), 1.81 (d, J=6.0 Hz, 2H), 1.71 (d, J=5.6 Hz, 2H). MS (ESI) m/z 414.1 [M+H]⁺

### Example 180. Synthesis of N-(6-methyl-5-(7-(methylamino)-1,6-naphthyridin-3-yl)pyridin-3-yl)-4,5,6,7-tetrahydro-1H-indazole-3-carboxamide (Compound 229)

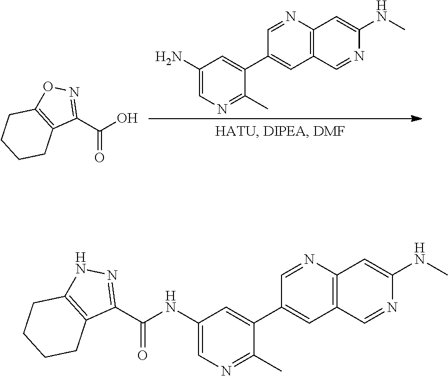 [SMILES: O=C(O)c1noc2c1CCCC2 ; CNc1cc2ncc(-c3cc(N)cnc3C)cc2cn1 ; CNc1cc2ncc(-c3cc(NC(=O)c4n[nH]c5c4CCCC5)cnc3C)cc2cn1]

A mixture of 4,5,6,7-tetrahydro-1H-indazole-3-carboxylic acid (33 mg, 0.2 mmol), 3-(5-amino-2-methylpyridin-3-yl)-N-methyl-1,6-naphthyridin-7-amine (53 mg, 0.2 mmol), 1-[bis(dimethylamino)methylene]-1H-1,2,3-triazolo[4,5-b]pyridinium 3-oxid hexafluorophosphate (152 mg, 0.4 mmol) and N,N-diisopropylethylamine (77 mg, 0.6 mmol) in N,N-dimethylformamide (5 mL) was stirred at room temperature for 2 hours. The reaction mixture was treated with water (30 mL) and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(6-methyl-5-(7-(methylamino)-1,6-naphthyridin-3-yl)pyridin-3-yl)-4,5,6,7-tetrahydro-1H-indazole-3-carboxamide (29.4 mg, 0.07 mmol, 36%) as a yellow solid. ¹H NMR (400 MHz, DMSO-d₆) δ 12.99 (s, 1H), 10.19 (s, 1H), 8.99 (s, 1H), 8.94 (d, J=1.9 Hz, 1H), 8.83 (t, J=9.5 Hz, 1H), 8.27 (dd, J=21.7, 1.9 Hz, 2H), 6.95 (q, J=4.9 Hz, 1H), 6.63 (s, 1H), 2.88 (d, J=4.9 Hz, 3H), 2.65 (dd, J=16.1, 10.8 Hz, 4H), 2.46 (s, 3H), 1.72 (dd, J=13.8, 6.7 Hz, 4H). MS (ESI) m/z 414.1 [M+H]⁺

### Example 181. Synthesis of (R)-6-methyl-N-(4-methyl-3-(7-(methylamino)-1,6-naphthyridin-3-yl)phenyl)-5,6,7,8-tetrahydroimidazo[1,2-a]pyridine-3-carboxamide and (S)-6-methyl-N-(4-methyl-3-(7-(methylamino)-1,6-naphthyridin-3-yl)phenyl)-5,6,7,8-tetrahydroimidazo[1,2-a]pyridine-3-carboxamide (Compounds 230 and 231)

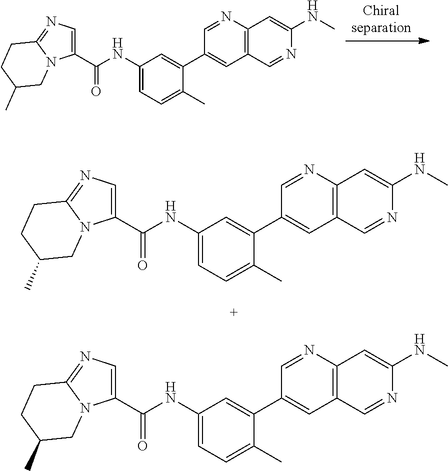 [SMILES: CNc1cc2ncc(-c3cc(NC(=O)c4cnc5n4CC(C)CC5)ccc3C)cc2cn1 ; CNc1cc2ncc(-c3cc(NC(=O)c4cnc5n4C[C@H](C)CC5)ccc3C)cc2cn1 ; CNc1cc2ncc(-c3cc(NC(=O)c4cnc5n4C[C@@H](C)CC5)ccc3C)cc2cn1]

The enantiomers of 6-methyl-N-(4-methyl-3-(7-(methylamino)-1,6-naphthyridin-3-yl)phenyl)-5,6,7,8-tetrahydroimidazo[1,2-a]pyridine-3-carboxamide (50 mg, 0.117 mmol) were separated by chiral SFC (instrument: Instrument: SFC-80 (Thar, Waters), Column: OD 20×250 mm, 10 μm (Daicel); Mobile phase: CO₂/EtOH (0.2% methanol ammonia)=65/35; Detection wavelength: 214 nm) to afford the first eluting enantiomer (retention time 12.15 min) which was arbitrarily assigned as (R)-6-methyl-N-(4-methyl-3-(7-(methylamino)-1,6-naphthyridin-3-yl)phenyl)-5,6,7,8-tetrahydroimidazo[1,2-a]pyridine-3-carboxamide (14.7 mg, 0.035 mmol) as a yellow solid. ¹H NMR (500 MHz, DMSO-d₆) δ 1.04 (d, J=6.5 Hz, 3H), 1.45-1.54 (m, 1H), 1.89 (d, J=13.0 Hz, 1H), 2.02 (s, 1H), 2.26 (s, 3H), 2.72-2.89 (m, 5H), 3.60 (t, J=11.0 Hz, 1H), 4.51 (dd, J₁=5.0 Hz, J₂=13.5 Hz, 1H), 6.62 (s, 1H), 6.88 (d, J=4.5 Hz, 1H), 7.30 (d, J=8.5 Hz, 1H), 7.65 (dd, J₁=1.0 Hz, J₂=8.25 Hz, 1H), 7.70 (d, J=2.0 Hz, 1H), 7.77 (s, 1H), 8.21 (d, J=2.0 Hz, 1H), 8.79 (d, J=2.0 Hz, 1H), 8.98 (s, 1H), 9.92 (s, 1H). MS (ESI) m/z 427.1 [M+H]⁺;
and the second eluting enantiomer (retention time 14.35 min) which was arbitrarily assigned as (S)-6-methyl-N-(4-methyl-3-(7-(methylamino)-1,6-naphthyridin-3-yl)phenyl)-5,6,7,8-tetrahydroimidazo[1,2-a]pyridine-3-carboxamide (14.9 mg, 0.035 mmol) as a yellow solid. ¹H NMR (500 MHz, DMSO-d₆) δ 1.04 (d, J=6.0 Hz, 3H), 1.45-1.54 (m, 1H), 1.89 (d, J=10.0 Hz, 1H), 2.01 (s, 1H), 2.26 (s, 3H), 2.72-2.79 (m, 1H), 2.87 (d, J=5.0 Hz, 4H), 3.60 (t, J=12.0 Hz, 1H), 4.51 (dd, J₁=4.5 Hz, J₂=13.5 Hz, 1H), 6.62 (s, 1H), 6.88 (d, J=5.0 Hz, 1H), 7.30 (d, 0.1=8.0 Hz, 1H), 7.65 (dd, J₁=2.0 Hz, J₂=8.5 Hz, 1H), 7.71 (d, J=2.0 Hz, 1H), 7.77 (s, 1H), 8.21 (d, J=2.0 Hz, 1H), 8.79 (d, J=2.0 Hz, 1H), 8.98 (s, 1H), 9.93 (s, 1H). MS (ESI) m/z 427.1 [M+H]⁺

### Example 182. Synthesis of N-(6-methyl-5-(7-(methylamino)-1,6-naphthyridin-3-yl)pyridin-3-yl)-6,7,8,9-tetrahydro-5H-imidazo[1,2-a]azepine-3-carboxamide (Compound 232)

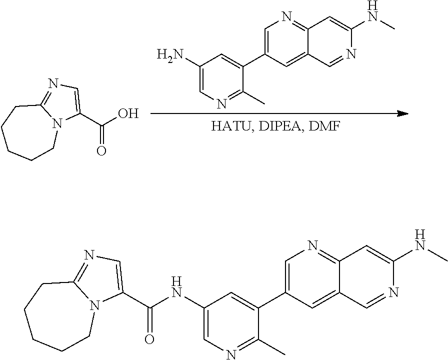 [SMILES: O=C(O)c1cnc2n1CCCCC2 ; CNc1cc2ncc(-c3cc(N)cnc3C)cc2cn1 ; CNc1cc2ncc(-c3cc(NC(=O)c4cnc5n4CCCCC5)cnc3C)cc2cn1]

A mixture of 6,7,8,9-tetrahydro-5H-imidazo[1,2-a]azepine-3-carboxylic acid (crude), 3-(5-amino-2-methylpyridin-3-yl)-N-methyl-1,6-naphthyridin-7-amine (26 mg, 0.1 mmol), 1-[bis(dimethylamino)methylene]-1H-1,2,3-triazolo[4,5-b]pyridinium 3-oxid hexafluorophosphate (152 mg, 0.4 mmol) and N,N-diisopropylethylamine (76 mg, 0.6 mmol) in N,N-dimethylformamide (5 mL) was stirred at room temperature for 2 hours. The reaction mixture was treated with water (30 mL) and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give N-(6-methyl-5-(7-(methylamino)-1,6-naphthyridin-3-yl)pyridin-3-yl)-6,7,8,9-tetrahydro-5H-imidazo[1,2-a]azepine-3-carboxamide (4.3 mg, 0.01 mmol, 10%) as a yellow solid. ¹H NMR (400 MHz, DMSO-d₆) δ 10.24 (s, 1H), 8.99 (s, 1H), 8.82 (dd, J=20.4, 2.3 Hz, 2H), 8.30 (d, J=2.1 Hz, 1H), 8.09 (d, J=2.4 Hz, 1H), 7.63 (s, 1H), 6.95 (d, J=4.8 Hz, 1H), 6.63 (s, 1H), 4.51 (s, 2H), 2.89 (d, J=8.8 Hz, 2H), 2.87 (d, J=4.9 Hz, 3H), 2.47 (s, 3H), 1.88-1.77 (m, 2H), 1.70-1.53 (m, 4H). MS (EST) m/z 428.2 [M+H]⁺

### Example 183. Synthesis of 1-methyl-N-(6-methyl-5-(7-(methylamino)-1,6-naphthyridin-3-yl)pyridin-3-yl)-4,5,6,7-tetrahydro-1H-indazole-3-carboxamide (Compound 233)

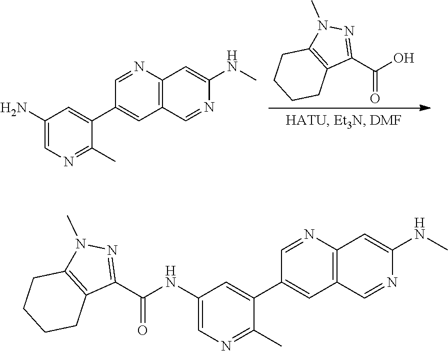

A solution of 1-methyl-4,5,6,7-tetrahydro-1H-indazole-3-carboxylic acid (27 mg, 0.15 mmol), 3-(5-amino-2-methylpyridin-3-yl)-N-methyl-1,6-naphthyridin-7-amine (40 mg, 0.15 mmol), 1-[bis(dimethylamino)methylene]-1H-1,2,3-triazolo[4,5-b]pyridinium 3-oxid hexafluorophosphate (171 mg, 0.45 mmol) and triethylamine (0.1 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 1-methyl-N-(6-methyl-5-(7-(methylamino)-1,6-naphthyridin-3-yl)pyridin-3-yl)-4,5,6,7-tetrahydro-1H-indazole-3-carboxamide (9.9 mg, 0.023 mmol, 16%) as a yellow solid. ¹H NMR (500 MHz, DMSO-d₆) δ 10.16 (s, 1H), 8.99 (s, 1H), 8.93 (s, 1H), 8.84 (s, 1H), 8.30 (s, 1H), 8.24 (s, 1H), 6.95 (s, 1H), 6.63 (s, 1H), 3.79 (s, 3H), 2.87 (d, J=4.9 Hz, 3H), 2.68 (t, J=6.0 Hz, 2H), 2.62 (t, J=6.2 Hz, 2H), 2.46 (s, 3H), 1.75 (d, J=5.3 Hz, 2H), 1.66 (d, J=5.1 Hz, 2H). MS (ESI) m/z 428.1 [M+H]⁺

### Example 184. Synthesis of N-(6-methyl-5-(7-(methylamino)-1,6-naphthyridin-3-yl)pyridin-3-yl)-4,5,6,7-tetrahydrobenzo[b]thiophene-3-carboxamide (Compound 234)

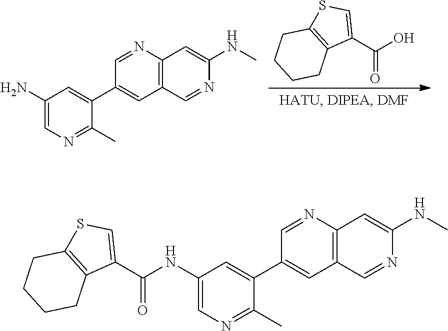

A mixture of 4,5,6,7-tetrahydrobenzo[b]thiophene-3-carboxylic acid (40 mg, 0.22 mmol), 3-(5-amino-2-methylpyridin-3-yl)-N-methyl-1,6-naphthyridin-7-amine (58 mg, 0.22 mmol), 1-[bis(dimethylamino)methylene]-1H-1,2,3-triazolo[4,5-b]pyridinium 3-oxid hexafluorophosphate (125 mg, 0.33 mmol) and N,N-diisopropylethylamine (57 mg, 0.44 mmol) in N,N-dimethylformamide (3 mL) was stirred at room temperature for 2 hours. The mixture was diluted with water (30 mL) and extracted with ethyl acetate (30 mL×3). The combined organic layer was washed with brine (30 mL), dried over anhydrous sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give N-(6-methyl-5-(7-(methylamino)-1,6-naphthyridin-3-yl)pyridin-3-yl)-4,5,6,7-tetrahydrobenzo[b]thiophene-3-carboxamide (7.0 mg, 0.016 mmol, 7%) as a yellow solid. ¹H NMR (400 MHz, DMSO-d₆) δ 10.27 (s, 1H), 8.99 (s, 1H), 8.82 (d, J=14.6 Hz, 2H), 8.30 (s, 1H), 8.12 (s, 1H), 8.01 (s, 1H), 6.96 (d, J=4.9 Hz, 1H), 6.63 (s, 1H), 2.87 (d, J=4.9 Hz, 3H), 2.76 (d, J=5.5 Hz, 4H), 2.46 (s, 3H), 1.75 (d, J=16.8 Hz, 4H). MS (ESI) m/z 430.0 [M+H]⁺

### Example 185. Synthesis of 6-methyl-N-(6-methyl-5-(7-(methylamino)-1,6-naphthyridin-3-yl)pyridin-3-yl)-4,5,6,7-tetrahydrobenzo[b]thiophene-3-carboxamide (Compound 235)

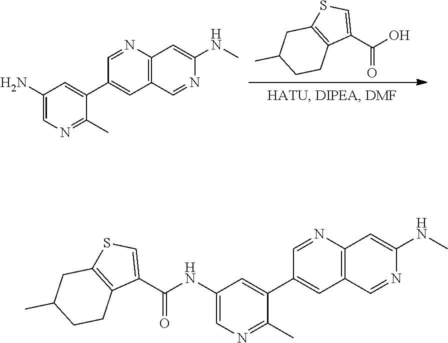

A mixture of 6-methyl-4,5,6,7-tetrahydrobenzo[b]thiophene-3-carboxylic acid (40 mg, 0.20 mmol), 3-(5-amino-2-methylpyridin-3-yl)-N-methyl-1,6-naphthyridin-7-amine (53 mg, 0.20 mmol), 1-[bis(dimethylamino)methylene]-1H-1,2,3-triazolo[4,5-b]pyridinium 3-oxid hexafluorophosphate (114 mg, 0.30 mmol) and N,N-diisopropylethylamine (52 mg, 0.40 mmol) in N,N-dimethylformamide (3 mL) was stirred at room temperature for 2 hours. The mixture was diluted with water (30 mL) and extracted with ethyl acetate (30 mL×3). The combined organic layer was washed with brine (30 mL), dried over anhydrous sodium sulfate, filtered, and concentrated. The residue was purified by prep-(Column: Xbridge 21.2×250 mm C18, 10 μm, Mobile Phase A: water (10 mM ammonium bicarbonate), B: acetonitrile) to give 6-methyl-N-(6-methyl-5-(7-(methylamino)-1,6-naphthyridin-3-yl)pyridin-3-yl)-4,5,6,7-tetrahydrobenzo[b]thiophene-3-carboxamide (9.0 mg, 0.02 mmol, 109%) as a yellow solid. ¹H NMR (400 MHz, DMSO-d₆) δ 10.26 (s, 1H), 8.99 (s, 1H), 8.82 (dd, J=13.8, 2.1 Hz, 2H), 8.28 (d, J=16.9 Hz, 1H), 8.12 (d, J=2.2 Hz, 1H), 8.02 (s, 1H), 6.95 (d, J=5.1 Hz, 1H), 6.63 (s, 1H), 2.96-2.82 (m, 5H), 2.68 (s, 1H), 2.46 (s, 3H), 2.39-2.27 (m, 1H), 1.85 (s, 2H), 1.33 (d, J=7.1 Hz, 1H), 1.04 (d, J=6.5 Hz, 3H). MS (ESI) m/z 444.1 [M+H]⁺

### Example 186. Synthesis of (S)-5-methyl-N-(4-methyl-3-(7-(methylamino)-1,6-naphthyridin-3-yl)phenyl)-4,5,6,7-tetrahydrobenzo[d]isoxazole-3-carboxamide and (R)-5-methyl-N-(4-methyl-3-(7-(methylamino)-1,6-naphthyridin-3-yl)phenyl)-4,5,6,7-tetrahydrobenzo[d]isoxazole-3-carboxamide (Compounds 236 and 237)

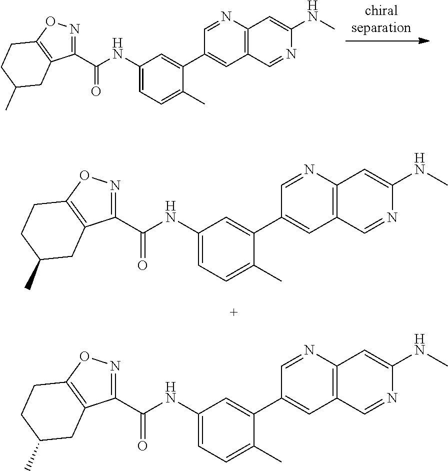 [SMILES: CNc1cc2ncc(-c3cc(NC(=O)c4noc5c4CC(C)CC5)ccc3C)cc2cn1 ; CNc1cc2ncc(-c3cc(NC(=O)c4noc5c4C[C@@H](C)CC5)ccc3C)cc2cn1 ; CNc1cc2ncc(-c3cc(NC(=O)c4noc5c4C[C@H](C)CC5)ccc3C)cc2cn1]

The enantiomers of 5-methyl-N-(4-methyl-3-(7-(methylamino)-1,6-naphthyridin-3-yl)phenyl)-4,5,6,7-tetrahydrobenzo[d]isoxazole-3-carboxamide (16 mg, 0.0374 mmol) were separated by chiral SFC (Column: OD 30×250 mm, 10 μm (Daicel); Mobile phase: CO₂/isopropanol (0.1% methanol ammonia)=50/50) to afford two enantiomers. The first eluting enantiomer (retention time 1.39 min) was further purified by prep-HPLC (Column: Phenomenex Luna 25×150 mm C18, 10 μm, Mobile Phase: A: water (formic acid), B: acetonitrile; B %: 32%-62% in 2 min) to give the pure enantiomer which was arbitrarily assigned as (S)-5-methyl-N-(4-methyl-3-(7-(methylamino)-1,6-naphthyridin-3-yl)phenyl)-4,5,6,7-tetrahydrobenzo[d]isoxazole-3-carboxamide (4.89 mg, 31%) as a green solid. ¹H NMR (400 MHz, DMSO-d₆) 10.64 (s, 1H), 8.98 (s, 1H), 8.79 (d, J=2.4 Hz, 1H), 8.22 (d, J=2.0 Hz, 1H), 7.81 (d, J=2.0 Hz, 1H), 7.72 (dd, J=2.0, 8.4 Hz, 1H), 7.33 (d, J=8.4 Hz, 1H), 6.88 (q, J=4.8 Hz, 1H), 6.62 (s, 1H), 2.87 (d, J=4.8 Hz, 3H), 2.83-2.72 (m, 3H), 2.27 (s, 3H), 2.14 (dd, J=9.6, 16.0 Hz, 1H), 1.94-1.78 (m, 2H), 1.54-1.39 (m, 1H), 1.04 (d, J=6.4 Hz, 3H). MS (ESI) m/z 428.2 [M+H]⁺;
The second eluting enantiomer (retention time 1.88 min) was further purified by prep-HPLC (Column: Phenomenex Luna 25×150 mm C18, 10 μm, Mobile Phase: A: water (formic acid), B: acetonitrile; B %: 32%-62% in 2 min) to give the pure enantiomer which was arbitrarily assigned as (R)-5-methyl-N-(4-methyl-3-(7-(methylamino)-1,6-naphthyridin-3-yl)phenyl)-4,5,6,7-tetrahydrobenzo[d]isoxazole-3-carboxamide (3.95 mg, 25%) as a green solid. ¹H NMR (400 MHz, DMSO-d₆) δ=10.64 (s, 1H), 8.98 (s, 1H), 8.79 (d, J=2.4 Hz, 1H), 8.22 (d, J=2.0 Hz, 1H), 7.81 (d, J=2.4 Hz, 1H), 7.72 (dd, J=2.4, 8.4 Hz, 1H), 7.33 (d, J=8.4 Hz, 1H), 6.88 (q, J=4.8 Hz, 1H), 6.62 (s, 1H), 2.87 (d, J=5.2 Hz, 3H), 2.83-2.70 (m, 3H), 2.27 (s, 3H), 2.14 (dd, J=9.2, 16.0 Hz, 1H), 1.91 (td, J=2.8, 9.8 Hz, 1H), 1.86-1.75 (m, 1H), 1.47 (dtd, J=5.6, 10.4, 12.9 Hz, 1H), 1.04 (d, J=6.4 Hz, 3H). MS (ESI) m/z 428.2 [M+H]⁺

### Example 187. Synthesis of 6-(difluoromethyl)-N-(4-methyl-3-(7-(methylamino)-1,6-naphthyridin-3-yl)phenyl)-5,6,7,8-tetrahydroimidazo[1,2-a]pyridine-3-carboxamide (Compound 238)

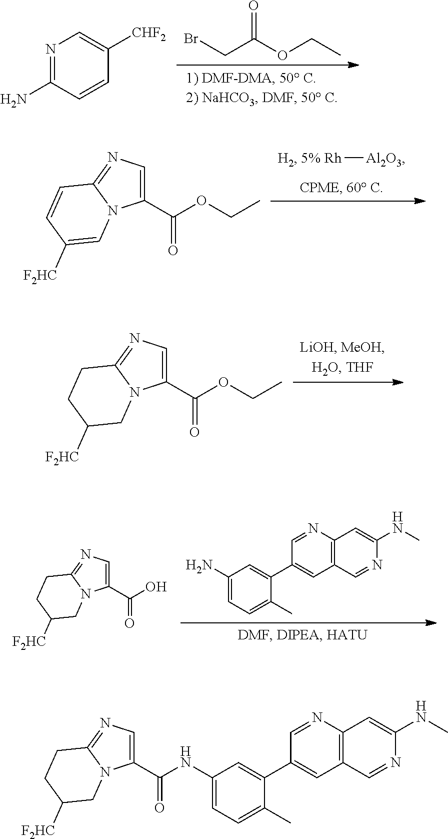

Step 1. A solution of 5-(difluoromethyl)pyridin-2-amine (500 mg, 1.6 mmol) in N,N-dimethyl formamide dimethyl acetal (20 mL) was stirred at 50° C. for 4 hours. After completion, the reaction mixture was concentrated. The residue was dissolved in N,N-dimethylformamide (15 mL). Ethyl 2-bromoacetate (530 mg, 3.2 mmol) and sodium bicarbonate (270 mg, 3.2 mmol) were added at room temperature. The resulting mixture was stirred at 50° C. for 16 hours. After cooling to room temperature, the reaction was diluted with ethyl acetate (20 mL×3), washed with saturated sodium bicarbonate aqueous solution (50 mL) and brine (40 mL), dried over sodium sulfate, filtered, and concentrated. The residue was purified by flash chromatography (petroleum ether/ethyl acetate=1/1) to give ethyl 6-(difluoromethyl)imidazo[1,2-a]pyridine-3-carboxylate (150 mg, 0.63 mmol, 39%) as a white solid. MS (ESI) m/z 241.2 [M+H]⁺

Step 2. To a solution of ethyl 6-(difluoromethyl)imidazo[1,2-a]pyridine-3-carboxylate (150 mg, 0.63 mmol) in cyclopentyl methyl ether (5 mL) at 60° C. was added rhodium-aluminum oxide (5%) (150 mg). The reaction mixture was stirred at 60° C. for 16 hours under hydrogen atmosphere. After cooling to room temperature, the reaction was filtered through celite. The filtrate was concentrated in vacuo. The residue was purified by flash chromatography (petroleum ether/ethyl acetate=1/1) to afford ethyl 6-(difluoromethyl)-5,6,7,8-tetrahydroimidazo[1,2-a]pyridine-3-carboxylate (75 mg, 0.31 mmol, 49%) as a white solid. MS (ESI) m/z 245.1 [M+H]⁺

Step 3. Lithium hydroxide monohydrate (39 mg, 0.93 mmol) was added to a solution of ethyl 6-(difluoromethyl)-5,6,7,8-tetrahydroimidazo[1,2-a]pyridine-3-carboxylate (75 mg, 0.31 mmol) in tetrahydrofuran (3 mL), methanol (3 mL), and water (3 mL) at 0° C. The reaction mixture was stirred at 25° C. for 2 hours. The reaction solution was diluted with water (20 mL) and concentrated. The aqueous layer was acidified to pH=3 with 4 N HCl. The aqueous phase was purified by prep-HPLC to give 6-(difluoromethyl)-5,6,7,8-tetrahydroimidazo[1,2-a]pyridine-3-carboxylic acid (25 mg, 0.12 mmol, 39%) as a white solid. MS (ESI) m/z 217.2 [M+H]⁺

Step 4. To a solution of 6-(difluoromethyl)-5,6,7,8-tetrahydroimidazo[1,2-a]pyridine-3-carboxylic acid (25 mg, 0.12 mmol) in N,N-dimethylformamide (4 mL) was added N,N-diisopropylethylamine (47 mg, 0.36 mmol), 1-[bis(dimethylamino)methylene]-1H-1,2,3-triazolo[4,5-b]pyridinium 3-oxid hexafluorophosphate (90 mg, 0.24 mmol) and 3-(5-amino-2-methylphenyl)-N-methyl-1,6-naphthyridin-7-amine (32 mg, 0.12 mmol). The mixture was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 6-(difluoromethyl)-N-(4-methyl-3-(7-(methylamino)-1,6-naphthyridin-3-yl)phenyl)-5,6,7,8-tetrahydroimidazo[1,2-a]pyridine-3-carboxamide (15 mg, 0.032 mmol, 27%) as a yellow solid. ¹H NMR (400 MHz, DMSO-d₆) δ 9.99 (s, 1H), 8.98 (s, 1H), 8.80 (d, J=2.0 Hz, 1H), 8.22 (d, J=1.6 Hz, 1H), 7.82 (s, 1H), 7.70-7.65 (m, 2H), 7.32-7.30 (m, 2H), 6.90-6.89 (m, 1H), 6.62 (s, 1H), 6.36 (d, J=4.8 Hz, 1H), 6.22-6.07 (m, 1H), 4.63-4.58 (m, 1H), 4.04-3.98 (m, 1H), 2.97-2.87 (m, 1H), 2.86 (s, 3H), 2.84-2.82 (m, 1H), 2.67-2.66 (m, 1H), 2.32 (s, 3H), 2.06-2.03 (m, 1H), 1.76-1.71 (m, 1H). MS (ESI) m/z 463.0 [M+H]⁺

### Example 188. Synthesis of 2-methyl-N-(6-methyl-5-(7-(methylamino)-1,6-naphthyridin-3-yl)pyridin-3-yl)-2,3-dihydrobenzofuran-7-carboxamide (Compound 239)

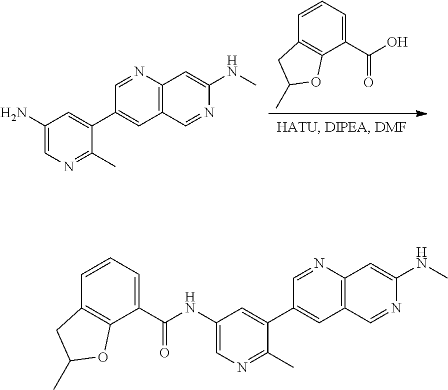

To a solution of 2-methyl-2,3-dihydrobenzofuran-7-carboxylic acid (25 mg, 0.14 mmol) in N,N-dimethylformamide (3 mL) was added N,N-diisopropylethylamine (54.2 mg, 0.42 mmol), 1-[bis(dimethylamino)methylene]-1H-1,2,3-triazolo[4,5-b]pyridinium 3-oxid hexafluorophosphate (79.8 mg, 0.21 mmol) and 3-(5-amino-2-methylpyridin-3-yl)-N-methyl-1,6-naphthyridin-7-amine (29.2 mg, 0.11 mmol). The mixture was stirred at room temperature for 2 hours. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 2-methyl-N-(6-methyl-5-(7-(methylamino)-1,6-naphthyridin-3-yl)pyridin-3-yl)-2,3-dihydrobenzofuran-7-carboxamide (9.6 mg, 0.023 mmol, 16%) as a yellow solid. ¹H NMR (400 MHz, DMSO-d₆) δ 9.92 (s, 1H), 9.00 (s, 1H), 8.87 (s, 2H), 8.34 (s, 1H), 8.15 (s, 1H), 7.63 (d, J=7.8 Hz, 1H), 7.43 (d, J=7.2 Hz, 1H), 6.98 (t, J=7.6 Hz, 2H), 6.63 (s, 1H), 5.34-4.98 (m, 1H), 3.46-3.40 (m, 2H), 2.88 (d, J=4.5 Hz, 3H), 2.48 (s, 3H), 1.51 (d, J=6.2 Hz, 3H). MS (ESI) m/z 426.1 [M+H]⁺

### Example 189. Synthesis of 5-methyl-N-(6-methyl-5-(7-(methylamino)-1,6-naphthyridin-3-yl)pyridin-3-yl)-1-(2,2,2-trifluoroethyl)-1H-pyrazole-4-carboxamide (Compound 240)

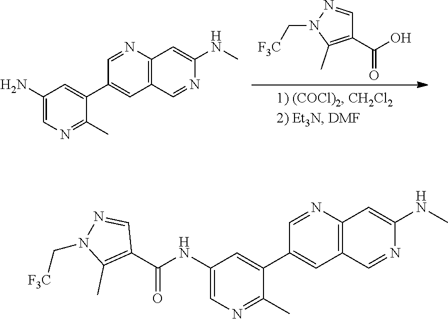 [SMILES: CNc1cc2ncc(-c3cc(N)cnc3C)cc2cn1 ; Cc1c(C(=O)O)cnn1CC(F)(F)F ; CNc1cc2ncc(-c3cc(NC(=O)c4cnn(CC(F)(F)F)c4C)cnc3C)cc2cn1]

To a solution of 5-methyl-1-(2,2,2-trifluoroethyl)-1H-pyrazole-4-carboxylic acid (177 mg, 0.85 mmol) in dichloromethane (6 mL) was added thionyl chloride (202.3 mg, 1.7 mmol) at 0° C. The mixture was stirred at room temperature for 10 minutes. After removing the solvent in vacuo, the crude product was dissolved in N,N-dimethylformamide (5 mL). 3-(5-Amino-2-methylpyridin-3-yl)-N-methyl-1,6-naphthyridin-7-amine (180.2 mg, 0.68 mmol) and triethylamine (171.7, 1.7 mmol) were added. The mixture was stirred at room temperature for 20 minutes. The reaction mixture was concentrated. The residue was purified by prep-HPLC to give 5-methyl-N-(6-methyl-5-(7-(methylamino)-1,6-naphthyridin-3-yl)pyridin-3-yl)-1-(2,2,2-trifluoroethyl)-1H-pyrazole-4-carboxamide (11.8 mg, 0.026 mmol, 3%) as a yellow solid. ¹H NMR (400 MHz, DMSO-d₆) δ 10.07 (s, 1H), 9.00 (s, 1H), 8.84 (dd, J=17.4, 2.1 Hz, 2H), 8.32 (s, 1H), 8.22 (s, 1H), 8.12 (s, 1H), 6.99 (s, 1H), 6.63 (s, 1H), 5.20 (q, J=8.9 Hz, 2H), 2.87 (d, J=4.6 Hz, 3H), 2.58 (s, 3H), 2.47 (s, 3H). MS (ESI) m/z 456.1 [M+H]⁺

### Example 190. Synthesis of 5-(tert-butyl)-N-(6-methyl-5-(7-(methylamino)-1,6-naphthyridin-3-yl)pyridin-3-yl)-4,5,6,7-tetrahydrobenzo[d]isoxazole-3-carboxamide (Compound 241)

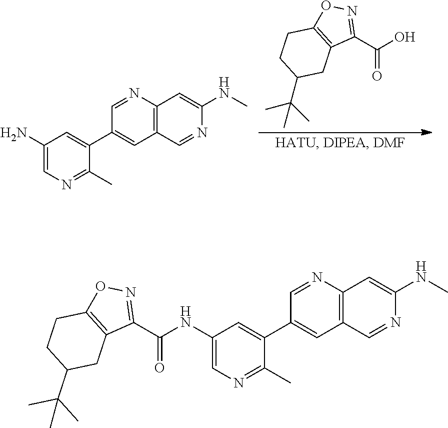

A mixture of 5-(tert-butyl)-4,5,6,7-tetrahydrobenzo[d]isoxazole-3-carboxylic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 24 hours. The mixture was purified by prep-HPLC (Column: Xbridge 21.2×250 mm C18, 10 μm, Mobile Phase A: water (10 mM ammonium bicarbonate), B: acetonitrile) to give 5-(tert-butyl)-N-(6-methyl-5-(7-(methylamino)-1,6-naphthyridin-3-yl)pyridin-3-yl)-4,5,6,7-tetrahydrobenzo[d]isoxazole-3-carboxamide (30 mg, 0.068 mmol, 45%). ¹H NMR (400 MHz, DMSO-d₆) 1H NMR δ 10.93 (s, 1H), 8.99 (s, 1H), 8.86 (dd, J=13.9, 2.3 Hz, 1H), 8.30 (d, J=2.0 Hz, 1H), 8.21 (d, J=2.3 Hz, 1H), 7.81 (d, J=13.0 Hz, 1H), 6.96 (q, J=4.9 Hz, 1H), 6.63 (s, 1H), 2.90 (dd, J=21.2, 4.7 Hz, 3H), 2.84-2.46 (m, 2H), 2.46-2.44 (m, 3H), 2.32-2.17 (m, 1H), 2.17-1.73 (m, 1H), 1.54-1.28 (m, 2H), 1.28-0.92 (m, 1H), 0.93 (d, J=4.1 Hz, 9H). MS (ESI) m/z 471 [M+H]⁺

### Example 191. Synthesis of N-(6-methyl-5-(7-(methylamino)-1,6-naphthyridin-3-yl)pyridin-3-yl)-4-(3,3,3-trifluoro-1-hydroxypropyl)picolinamide (Compound 242)

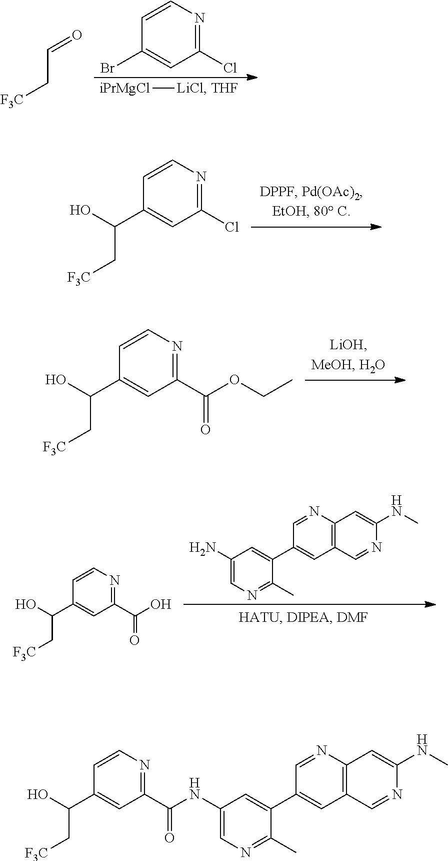

Step 1. To a solution of 3,3,3-trifluoropropanal (500 mg, 4.46 mmol) and 4-bromo-2-chloropyridine (191 mg, 2.23 mmol) in tetrahydrofuran (2 mL) was added isopropyl magnesium chloride lithium chloride complex (1.7 mL) at 0° C. dropwise under nitrogen atmosphere. The reaction solution was stirred for 16 hours at room temperature. The mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 1-(2-chloropyridin-4-yl)-3,3,3-trifluoropropan-1-ol (62 mg, 0.28 mmol, 12%) as a white solid. MS (ESI) m/z 226.1 [M+H]⁺

Step 2. To a solution of 1-(2-chloropyridin-4-yl)-3,3,3-trifluoropropan-1-ol (62 mg, 0.28 mmol) in ethanol (3 mL) were added palladium(II)acetate (12.6 mg, 0.056 mmol), 1,1′-bis(diphenylphosphino)ferrocene (77.56 mg, 0.14 mmol) and triethylamine (113.12 mg, 1.12 mmol). The mixture was stirred at 80° C. for 16 hours under carbon monoxide atmosphere. After cooling to room temperature, the reaction mixture was quenched with water/ice (100 mL) and extracted with ethyl acetate (3×40 mL). The combined organic layers were washed with brine (2×100 mL) and dried over anhydrous sodium sulfate. After filtration, the filtrate was concentrated reduced pressure. The residue was purified by flash chromatography (silica, ethyl acetate/petroleum ether=1/1) to afford ethyl 4-(3,3,3-trifluoro-1-hydroxypropyl)picolinate (42 mg, 0.16 mmol, 57%) as a yellow oil. MS (ESI) m/z 264.1 [M+H]⁺

Step 3. A solution of ethyl 4-(3,3,3-trifluoro-1-hydroxypropyl)picolinate (42 mg, 0.16 mmol) and lithium hydroxide (20.2 mg, 0.48 mmol) in methanol (2 mL) and water (2 mL) was stirred for 2 hours at room temperature. The mixture was adjusted to pH=3 with 10% hydrochloric acid and then purified by prep-HPLC (Column: Welch Xtimate 21.2×250 mm C18, 10 μm, Mobile Phase: A: water (10 mM formic acid), B: acetonitrile; B %: 30%-70% in 15 min) to afford 4-(3,3,3-trifluoro-1-hydroxypropyl)picolinic acid (15 mg, 0.06 mmol, 40%) as a white solid. MS (ESI) m/z 236.1 [M+H]⁺

Step 4. To a solution of 4-(3,3,3-trifluoro-1-hydroxypropyl)picolinic acid (15 mg, 0.06 mmol) in N,N-dimethylformamide (2 mL) were added 3-(5-amino-2-methylpyridin-3-yl)-N-methyl-1,6-naphthyridin-7-amine (12.7 mg, 0.05 mmol), 1-[bis(dimethylamino)methylene]-1H-1,2,3-triazolo[4,5-b]pyridinium 3-oxid hexafluorophosphate (34.2 mg, 0.09 mmol) and N,N-diisopropylethylamine (23.22 mg, 0.18 mmol) at room temperature. The reaction mixture was stirred room temperature for 3 hours. The mixture purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afforded N-(6-methyl-5-(7-(methylamino)-1,6-naphthyridin-3-yl)pyridin-3-yl)-4-(3,3,3-trifluoro-1-hydroxypropyl)picolinamide (20 mg, 0.04 mmol, 69%) as a yellow solid. ¹H NMR (400 MHz, DMSO-d₆) δ 10.98 (s, 1H), 9.06 (d, J=2.2 Hz, 1H), 9.00 (s, 1H), 8.88 (d, J=2.0 Hz, 1H), 8.73 (d, J=4.9 Hz, 1H), 8.33 (d, J=7.0 Hz, 2H), 8.24 (s, 1H), 7.74 (d, J=5.9 Hz, 1H), 7.00 (s, 1H), 6.64 (s, 1H), 6.08 (d, J=5.3 Hz, 1H), 5.07 (s, 1H), 2.88 (d, J=4.4 Hz, 3H), 2.72 (dd, J=23.3, 11.2 Hz, 2H), 2.50-2.50 (m, 3H). MS (ESI) m/z 483.1 [M+H]⁺

### Example 192. Synthesis of N-(4-methyl-3-(7-(methylamino)-1,6-naphthyridin-3-yl)phenyl)-6-(trifluoromethyl)-5,6,7,8-tetrahydroimidazo[1,2-a]pyridine-3-carboxamide (Compound 243)

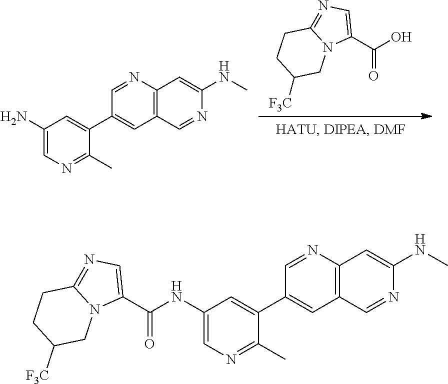 [SMILES: CNc1cc2ncc(-c3cc(N)cnc3C)cc2cn1 ; O=C(O)c1cnc2n1CC(C(F)(F)F)CC2 ; CNc1cc2ncc(-c3cc(NC(=O)c4cnc5n4CC(C(F)(F)F)CC5)cnc3C)cc2cn1]

To a solution of 6-(trifluoromethyl)-5,6,7,8-tetrahydroimidazo[1,2-a]pyridine-3-carboxylic acid (50 mg, 0.21 mmol) in N,N-dimethylformamide (4 mL) were added N,N-diisopropylethylamine (82 mg, 0.64 mmol), 1-[bis(dimethylamino)methylene]-1H-1,2,3-triazolo[4,5-b]pyridinium 3-oxid hexafluorophosphate (121 mg, 0.32 mmol) and 3-(5-amino-2-methylpyridin-3-yl)-N-methyl-1,6-naphthyridin-7-amine (56 mg, 0.21 mmol) at room temperature. The mixture was stirred at room temperature for 1 hour. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(4-methyl-3-(7-(methylamino)-1,6-naphthyridin-3-yl)phenyl)-6-(trifluoromethyl)-5,6,7,8-tetrahydroimidazo[1,2-a]pyridine-3-carboxamide (2.1 mg, 0.004 mmol) as a yellow solid. ¹H NMR (400 MHz, DMSO-d₆) δ 1.85-1.96 (m, 1H), 2.17 (d, J=12.4 Hz, 1H), 2.47 (s, 3H), 2.87 (d, J=4.8 Hz, 3H), 2.90-3.00 (m, 2H), 3.20 (s, 1H), 4.12 (t, J=11.2 Hz, 1H), 4.72 (q, J=8.0 Hz, 1H), 6.63 (s, 1H), 6.96 (q, J=4.4 Hz, 1H), 7.87 (s, 1H), 8.11 (s, 1H), 8.30 (s, 1H), 8.79 (s, 1H), 8.85 (s, 1H), 8.99 (s, 1H), 10.23 (s, 1H). MS (ESI) m/z 482.1 [M+H]⁺

### Example 193. Synthesis of 5-methyl-N-(6-methyl-5-(7-(methylamino)-1,6-naphthyridin-3-yl)pyridin-3-yl)-4,5,6,7-tetrahydro-1H-pyrazolo[4,3-c]pyridine-3-carboxamide (Compound 244)

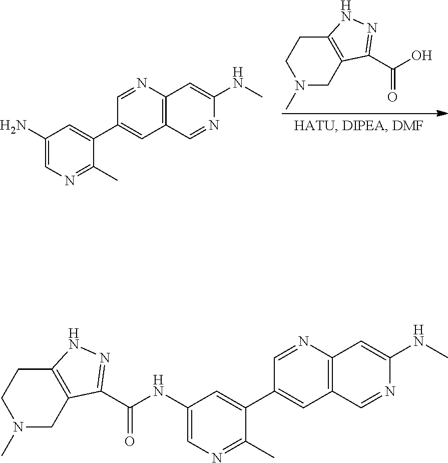

To a solution of 5-methyl-4,5,6,7-tetrahydro-1H-pyrazolo[4,3-c]pyridine-3-carboxylic acid (30 mg, 0.16 mmol) in N,N-dimethylformamide (1 mL) were added N,N-diisopropylethylamine (32 mg, 0.25 mmol), 3-(5-amino-2-methylpyridin-3-yl)-N-methyl-1,6-naphthyridin-7-amine (42 mg, 0.16 mmol) and 1-[bis(dimethylamino)methylene]-1H-1,2,3-triazolo[4,5-b]pyridinium 3-oxid hexafluorophosphate (73 mg, 0.19 mmol) at room temperature. The reaction was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5-methyl-N-(6-methyl-5-(7-(methylamino)-1,6-naphthyridin-3-yl)pyridin-3-yl)-4,5,6,7-tetrahydro-1H-pyrazolo[4,3-c]pyridine-3-carboxamide (13.8 mg, 0.03 mmol, 20%) as a yellow solid. ¹H NMR (400 MHz, DMSO-d₆) δ 13.15 (s, 1H), 10.29 (s, 1H), 8.99 (s, 1H), 8.94 (d, J=1.6 Hz, 1H), 8.44 (d, J=2.4 Hz, 1H), 8.30 (d, J=2.0 Hz, 1H), 8.24 (d, J=2.4 Hz, 1H), 6.96-6.95 (m, 1H), 6.63 (s, 1H), 3.55 (s, 2H), 2.87 (s, J=4.8 Hz, 3H), 2.74-2.73 (m, 2H), 2.64 (s, 2H), 2.46 (s, 3H), 2.38 (s, 3H). MS (ESI) m/z 429.1 [M+H]⁺

### Example 194. Synthesis of N-(6-methyl-5-(7-(methylamino)-1,6-naphthyridin-3-yl)pyridin-3-yl)-5,6,7,8-tetrahydro-4H-cyclohepta[d]isoxazole-3-carboxamide (Compound 245)

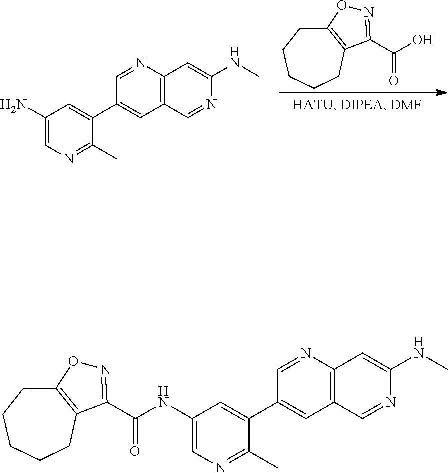

To a solution of 5,6,7,8-tetrahydro-4H-cyclohepta[d]isoxazole-3-carboxylic acid (30 mg 0.17 mmol) in N,N-dimethylformamide (4 mL) were added N,N-diisopropylethylamine (64 mg, 0.49 mmol), 1-[bis(dimethylamino)methylene]-1H-1,2,3-triazolo[4,5-b]pyridinium 3-oxid hexafluorophosphate (94 mg, 0.25 mmol) and 3-(5-amino-2-methylpyridin-3-yl)-N-methyl-1,6-naphthyridin-7-amine (44 mg, 0.17 mmol). The mixture was stirred at room temperature for 1 hour. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(6-methyl-5-(7-(methylamino)-1,6-naphthyridin-3-yl)pyridin-3-yl)-5,6,7,8-tetrahydro-4H-cyclohepta[d]isoxazole-3-carboxamide (27.7 mg, 0.065 mmol, 39%) as a yellow solid. ¹H NMR (400 MHz, DMSO-d₆) δ 1.64-1.70 (m, 4H), 1.80 (d, J=4.0 Hz, 2H), 2.47 (s, 3H), 2.73 (t, J=5.6 Hz, 2H), 2.87 (d, J=4.8 Hz, 3H), 2.94 (t, J=5.6 Hz, 2H), 6.63 (s, 1H), 6.95 (q, J=4.8 Hz, 1H), 8.16 (d, J=2.0 Hz, 1H), 8.30 (d, J=1.6 Hz, 1H), 8.85 (dd, J₁=2.0 Hz, J₂=9.6 Hz, 2H), 8.98 (s, 1H), 10.91 (s, 1H). MS (ESI) m/z 429.1 [M+H]⁺

### Example 195. Synthesis of N-(6-methyl-5-(7-(methylamino)-1,6-naphthyridin-3-yl)pyridin-3-yl)-6,7-dihydro-4H-pyrano[3,4-d]isoxazole-3-carboxamide (Compound 246)

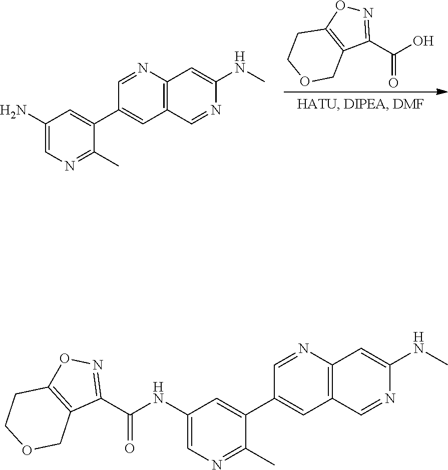

To a solution of 6,7-dihydro-4H-pyrano[3,4-d]isoxazole-3-carboxylic acid (30 mg, 0.18 mmol) in N,N-dimethylformamide (4 mL) were added N,N-diisopropylethylamine (69 mg, 0.53 mmol), 1-[bis(dimethylamino)methylene]-1H-1,2,3-triazolo[4,5-b]pyridinium 3-oxid hexafluorophosphate (101 mg, 0.27 mmol) and 3-(5-amino-2-methylpyridin-3-yl)-N-methyl-1,6-naphthyridin-7-amine (47 mg, 0.18 mmol). The mixture was stirred at room temperature for 1 hour. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(6-methyl-5-(7-(methylamino)-1,6-naphthyridin-3-yl)pyridin-3-yl)-6,7-dihydro-4H-pyrano[3,4-d]isoxazole-3-carboxamide (27.9 mg, 0.067 mmol, 38%) as a yellow solid. ¹H NMR (400 MHz, DMSO-d₆) δ 2.48 (s, 3H), 2.87 (d, J=4.4 Hz, 3H), 2.94 (t, J=4.8 Hz, 2H), 3.92 (t, J=5.2 Hz, 2H), 4.70 (s, 2H), 6.62 (s, 1H), 6.99 (d, J=5.2 Hz, 1H), 8.19 (d, J=2.0 Hz, 1H), 8.31 (s, 1H), 8.85 (d, J=1.6 Hz, 1H), 8.90 (d, J=2.0 Hz, 1H), 8.99 (s, 1H), 11.07 (s, 1H). MS (ESI) m/z 417.1 [M+H]⁺

### Example 196. Synthesis of 5,5-difluoro-N-(6-methyl-5-(7-(methylamino)-1,6-naphthyridin-3-yl)pyridin-3-yl)-4,5,6,7-tetrahydrobenzo[d]isoxazole-3-carboxamide (Compound 247)

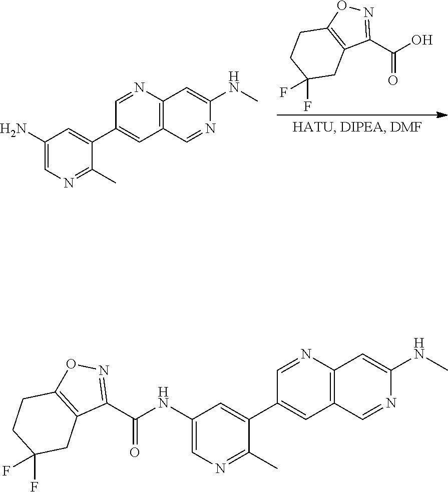

A solution of 5,5-difluoro-4,5,6,7-tetrahydrobenzo[d]isoxazole-3-carboxylic acid (17 mg, 0.08 mmol), 3-(5-amino-2-methylpyridin-3-yl)-N-methyl-1,6-naphthyridin-7-amine (22 mg, 0.08 mmol), I-[bis(dimethylamino)methylene]-1H-1,2,3-triazolo[4,5-b]pyridinium 3-oxid hexafluorophosphate (61 mg, 0.16 mmol) and N,N-diisopropylethylamine (31 mg, 0.24 mmol) in N,N-dimethylformamide (1 mL) was stirred at room temperature for 2 hours. The solution was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5,5-difluoro-N-(6-methyl-5-(7-(methylamino)-1,6-naphthyridin-3-yl)pyridin-3-yl)-4,5,6,7-tetrahydrobenzo[d]isoxazole-3-carboxamide (12.4 mg, 0.027 mmol, 34%) as a yellow solid. ¹H NMR (400 MHz, DMSO-d₆) δ 11.05 (s, 1H), 8.99 (s, 1H), 8.89 (d, J=2.4 Hz, 1H), 8.84 (d, J=2.4 Hz, 1H), 8.30 (d, J=2.0 Hz, 1H), 8.18 (d, J=2.8 Hz, 1H), 6.97-6.96 (m, 1H), 6.63 (s, 1H), 3.23 (t, J=14.0 Hz, 2H), 3.04 (t, J=6.4 Hz, 2H), 2.86 (d, J=5.2 Hz, 3H), 2.48 (s, 3H), 2.46-2.37 (m, 2H). MS (ESI) m/z 451.2 [M+H]⁺

### Example 197. Synthesis of (R)-4-(2,2-difluoro-1-hydroxypropyl)-N-(6-methyl-5-(7-(methylamino)-1,6-naphthyridin-3-yl)pyridin-3-yl)picolinamide and (S)-4-(2,2-difluoro-1-hydroxypropyl)-N-(6-methyl-5-(7-(methylamino)-1,6-naphthyridin-3-yl)pyridin-3-yl)picolinamide (Compounds 248 and 249)

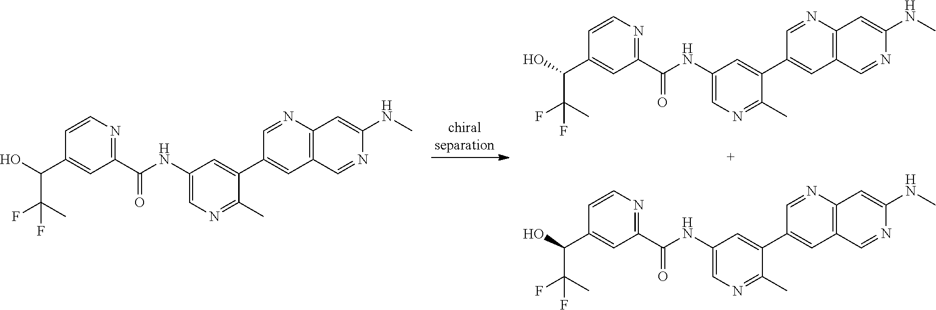 [SMILES: CNc1cc2ncc(-c3cc(NC(=O)c4cc(C(O)C(C)(F)F)ccn4)cnc3C)cc2cn1 ; CNc1cc2ncc(-c3cc(NC(=O)c4cc([C@@H](O)C(C)(F)F)ccn4)cnc3C)cc2cn1 ; CNc1cc2ncc(-c3cc(NC(=O)c4cc([C@H](O)C(C)(F)F)ccn4)cnc3C)cc2cn1]

The enantiomers of 4-(2,2-difluoro-1-hydroxypropyl)-N-(6-methyl-5-(7-(methylamino)-1,6-naphthyridin-3-yl)pyridin-3-yl)picolinamide were separated by chiral SFC (Instrument: SFC-150 (Waters); Column: OJ 20×250 mm, 10 μm (Daicel); Mobile phase: CO₂/MeOH (0.2% methanol ammonia)=40/60; Detection wavelength: 214 nm) to afford the first eluting enantiomer (retention time 2.2 min) which was arbitrarily assigned as (R)-4-(2,2-difluoro-1-hydroxypropyl)-N-(6-methyl-5-(7-(methylamino)-1,6-naphthyridin-3-yl)pyridin-3-yl)picolinamide (22 mg, 0.060 mmol, 29%) as a yellow solid. ¹H NMR (500 MHz, DMSO-d₆) δ 10.99 (s, 1H), 9.05 (d, J=2.2 Hz, 1H), 8.99 (s, 1H), 8.87 (d, J=2.1 Hz, 1H), 8.76 (d, J=5.0 Hz, 1H), 8.32 (d, J=2.2 Hz, 2H), 8.24 (s, 1H), 7.72 (d, J=4.7 Hz, 1H), 6.97 (d, J=5.0 Hz, 1H), 6.70 (d, J=5.5 Hz, 1H), 6.64 (s, 1H), 5.01 (dd, J=13.6, 6.1 Hz, 1H), 2.88 (d, J=4.9 Hz, 3H), 2.50 (d, J=1.9 Hz, 3H), 1.59 (t, J=19.1 Hz, 3H). MS (ESI) m/z 465 [M+H]⁺;
and the second eluting enantiomer (retention time 2.8 min) which was arbitrarily assigned as (S)-4-(2,2-difluoro-1-hydroxypropyl)-N-(6-methyl-5-(7-(methylamino)-1,6-naphthyridin-3-yl)pyridin-3-yl)picolinamide (22 mg, 0.083 mmol, 40%) as a yellow solid. ¹H NMR (500 MHz, DMSO-d₆) δ 10.99 (s, 1H), 9.05 (d, J=2.2 Hz, 1H), 8.99 (s, 1H), 8.87 (d, J=2.1 Hz, 1H), 8.76 (d, J=5.0 Hz, 1H), 8.32 (d, J=2.2 Hz, 2H), 8.24 (s, 1H), 7.72 (d, J=4.7 Hz, 1H), 6.97 (d, J=5.0 Hz, 1H), 6.70 (d, J=5.5 Hz, 1H), 6.64 (s, 1H), 5.01 (dd, J=13.6, 6.1 Hz, 1H), 2.88 (d, J=4.9 Hz, 3H), 2.50 (d, J=1.9 Hz, 3H), 1.59 (t, J=19.1 Hz, 3H). MS (ESI) m/z 465[M+H]⁺

### Example 198. Synthesis of 6-(1,1-difluoro-2-(methylamino)ethyl)-N-(6-methyl-5-(7-(methylamino)-1,6-naphthyridin-3-yl)pyridin-3-yl)picolinamide (Compound 250)

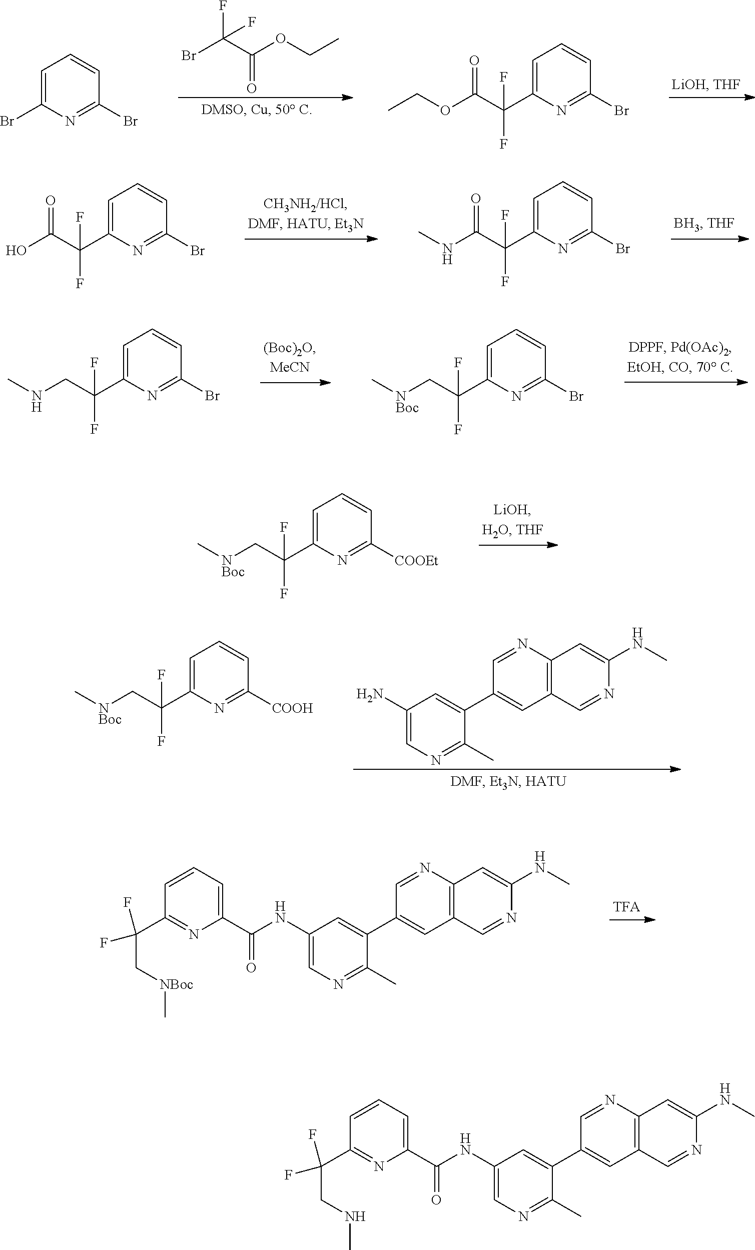

Step 1. To a solution of 2,6-dibromopyridine (5 g, 21.1 mmol) in dimethyl sulfoxide (20 mL) was added ethyl 2-bromo-2,2-difluoroacetate (4.7 g, 23.2 mmol) and copper powder (2.7 g, 42.2 mmol). The mixture was stirred at 50° C. for 6 hours. The mixture were concentrated and purified by flash chromatography (silica, petroleum ether/ethyl acetate=1/1) to give ethyl 2-(6-bromopyridin-2-yl)-2,2-difluoroacetate (4.2 g, 18 mmol, 71%) as a colorless oil. MS (ESI) m/z 280.1 [M+H]⁺

Step 2. A mixture of ethyl 2-(6-bromopyridin-2-yl)-2,2-difluoroacetate (1 g, 3.58 mmol) and lithium hydroxide monohydrate (451 mg, 10.74 mmol) in tetrahydrofuran (10 mL) and water (3 mL) was stirred at room temperature for 2 hours. The resulting mixture was diluted with water (20 mL) and acidified with hydrochloric acid to pH=5. The mixture was filtered and concentrated to give 2-(6-bromopyridin-2-yl)-2,2-difluoroacetic acid (820 mg, 3.27 mmol, 91%) as a white oil. MS (ESI) m/z 252.9 [M+H]⁺

Step 3. A solution of 2-(6-bromopyridin-2-yl)-2,2-difluoroacetic acid (820 mg, 3.27 mmol), methylamine hydrochloride (222 mg, 3.27 mmol), 1-[bis(dimethylamino)methylene]-1H-1,2,3-triazolo[4,5-b]pyridinium 3-oxid hexafluorophosphate (3.7 g, 9.81 mmol) and triethylamine (0.3 mL) in N,N-dimethylformamide (10 mL) was stirred at room temperature for 2 hours. The crude residue was purified by flash chromatography (silica, petroleum ether/ethyl acetate=1/1) to give 2-(6-bromopyridin-2-yl)-2,2-difluoro-N-methylacetamide (580 mg, 2.19 mmol, 67%). MS (ESI) m/z 267.0 [M+H]⁺

Step 4. A solution of 2-(6-bromopyridin-2-yl)-2,2-difluoro-N-methylacetamide (580 mg, 2.19 mmol) in borane-tetrahydrofuran complex (1 M in tetrahydrofuran, 5 mL) was stirred at room temperature for 16 hours. The resulting mixture was dilution with water, then extracted with ethyl acetate (10 mL×3). The combined organic phases were dried over sodium sulfate, filtered, and concentrated. The crude residue was purified by flash chromatography (silica, petroleum ether/ethyl acetate=1/1) to give 2-(6-bromopyridin-2-yl)-2,2-difluoro-N-methylethan-1-amine (170 mg, 0.68 mmol, 31%) as a yellow oil. MS (ESI) m/z 252.9 [M+H]⁺

Step 5. To a solution of 2-(6-bromopyridin-2-yl)-2,2-difluoro-N-methylethan-1-amine (170 mg, 0.68 mmol) in acetonitrile (5 mL) were added di-tert-butyl dicarbonate (178 mg, 0.82 mmol) and 4-dimethylaminopyridine (8 mg, 0.068 mmol). The mixture was stirred at room temperature for 4 hours. The mixture were concentrated and purified by flash chromatography (silica, petroleum ether/ethyl acetate=1/1) to give tert-butyl (2-(6-bromopyridin-2-yl)-2,2-difluoroethyl)(methyl)carbamate (195 mg, 0.56 mmol, 82%) as a yellow oil. MS (ESI) m/z 297.0 [M+H]⁺

Step 6. A solution of tert-butyl (2-(6-bromopyridin-2-yl)-2,2-difluoroethyl)(methyl)carbamate (195 mg, 0.56 mmol), 1,1′-bis(diphenylphosphino)ferrocene (61 mg, 0.11 mmol), palladium(II)acetate (13 mg, 0.056 mmol) and triethylamine (0.2 mL) in ethanol (5 mL) was stirred at 70° C. for 16 hours under carbon monoxide atmosphere. The crude residue was by purified flash chromatography (silica, petroleum ether/ethyl acetate=1/1) to give ethyl 6-(2-((tert-butoxycarbonyl)methyl)amino)-1,1-difluoroethyl)picolinate (135 mg, 0.39 mmol, 70%) as a white solid. MS (ESI) m/z 345.1 [M+H]⁺

Step 7. A mixture of ethyl 6-(2-((tert-butoxycarbonyl)(methyl)amino)-1,1-difluoroethyl)picolinate (135 mg, 0.39 mmol) and lithium hydroxide monohydrate (49 mg, 1.17 mmol) in tetrahydrofuran (5 mL) and water (1 mL) was stirred at room temperature for 2 hours. The resulting mixture was diluted with water (20 mL) and acidified with hydrochloric acid to pH=5. The mixture was filtered and concentrated to give 6-(2-((tert-butoxycarbonyl)(methyl)amino)-1,1-difluoroethyl)picolinic acid (103 mg, 0.33 mmol, 84%) as a white solid. MS (ESI) m/z 261.1 [M+H]⁺

Step 8. A solution of 6-(2-((tert-butoxycarbonyl)(methyl)amino)-1,1-difluoroethyl)picolinic acid (50 mg, 0.16 mmol), 3-(5-amino-2-methylpyridin-3-yl)-N-methyl-1,6-naphthyridin-7-amine (42 mg, 0.16 mmol), 1-[bis(dimethylamino)methylene]-1H-1,2,3-triazolo[4,5-b]pyridinium 3-oxid hexafluorophosphate (182 mg, 0.48 mmol) and triethylamine (0.1 mL) in N,N-dimethylformamide (3 mL) was stirred at room temperature for 3 hours. The crude residue was purified flash chromatography (silica, petroleum ether/ethyl acetate=1/8) to give tert-butyl(2,2-difluoro-2-(6-((6-methyl-5-(7-(methylamino)-1,6-naphthyridin-3-yl)pyridin-3-yl)carbamoyl)pyridin-2-yl)ethyl)(methyl)carbamate (75 mg, 0.13 mmol, 83%) as a yellow solid. MS (ESI) m/z 564.2 [M+H]⁺

Step 9. A mixture of tert-butyl (2,2-difluoro-2-(6-((6-methyl-5-(7-(methylamino)-1,6-naphthyridin-3-yl)pyridin-3-yl)carbamoyl)pyridin-2-yl)ethyl)(methyl)carbamate (75 mg, 0.13 mmol) in trifluoroacetic acid (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 6-(1,1-difluoro-2-(methylamino)ethyl)-N-(6-methyl-5-(7-(methylamino)-1,6-naphthyridin-3-yl)pyridin-3-yl)picolinamide (18.2 mg, 0.04 mmol, 30%) as a yellow solid. ¹H NMR (500 MHz, DMSO-d6) δ 10.58 (s, 1H), 9.03 (d, J=2.4 Hz, 1H), 9.00 (s, 1H), 8.88 (d, J=2.3 Hz, 1H), 8.33 (d, J=2.1 Hz, 1H), 8.25 (dt, J=9.8, 4.6 Hz, 3H), 7.97 (d, J=7.4 Hz, 1H), 6.95 (q, J=4.9 Hz, 1H), 6.64 (s, 1H), 3.61 (t, J=14.7 Hz, 2H), 2.88 (d, J=5.0 Hz, 3H), 2.51 (s, 3H), 2.34 (s, 3H). MS (ESI) m/z 464.1 [M+H]⁺

### Example 199. Synthesis of 6-(difluoromethyl)-N-(6-methyl-5-(7-(methylamino)-1,6-naphthyridin-3-yl)pyridin-3-yl)-5,6,7,8-tetrahydroimidazo[1,2-a]pyridine-3-carboxamide (Compound 251)

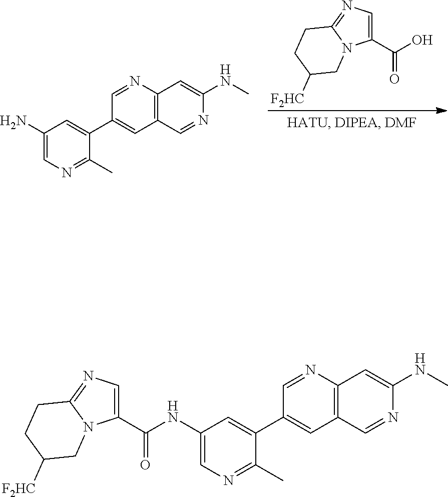 [SMILES: CNc1cc2ncc(-c3cc(N)cnc3C)cc2cn1 ; O=C(O)c1cnc2n1CC(C(F)F)CC2 ; CNc1cc2ncc(-c3cc(NC(=O)c4cnc5n4CC(C(F)F)CC5)cnc3C)cc2cn1]

To a solution of 6-(difluoromethyl)-5,6,7,8-tetrahydroimidazo[1,2-a]pyridine-3-carboxylic acid (30 mg, 0.14 mmol) in N,N-dimethylformamide (4 mL) were added N,N-diisopropylethylamine (55 mg, 0.42 mmol), 1-[bis(dimethylamino)methylene]-1H-1,2,3-triazolo[4,5-b]pyridinium 3-oxid hexafluorophosphate (105 mg, 0.28 mmol) and 3-(5-amino-2-methylpyridin-3-yl)-N-methyl-1,6-naphthyridin-7-amine (37 mg, 0.14 mmol). The mixture was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 6-(difluoromethyl)-N-(6-methyl-5-(7-(methylamino)-1,6-naphthyridin-3-yl)pyridin-3-yl)-5,6,7,8-tetrahydroimidazo[1,2-a]pyridine-3-carboxamide (10 mg, 0.022 mmol, 15%) as a yellow solid. ¹H NMR (400 MHz, DMSO-d₆) δ 10.22 (s, 1H), 8.99 (s, 1H), 8.85 (d, J=2.0 Hz, 1H), 8.81 (d, J=2.4 Hz, 1H), 8.30 (d, J=2.0 Hz, 1H), 8.11 (d, J=2.0 Hz, 1H), 7.86 (s, 1H), 6.97 (s, 1H), 6.63 (s, 1H), 6.21-6.07 (m, 1H), 4.63-4.58 (m, 1H), 4.04-3.98 (m, 1H), 2.97-2.87 (m, 1H), 2.94 (s, 3H), 2.84-2.82 (m, 1H), 2.67-2.66 (m, 1H), 2.47 (s, 3H), 2.41-2.39 (m, 1H), 1.76-1.71 (m, 1H). MS (ESI) m/z 464.1 [M+H]⁺

### Example 200. Synthesis of (4aS,5aS)—N-(6-methyl-5-(7-(methylamino)-1,6-naphthyridin-3-yl)pyridin-3-yl)-4,4a,5,5a-tetrahydro-1H-cyclopropa[4,5]cyclopenta[1,2-c]pyrazole-3-carboxamide (Compound 252)

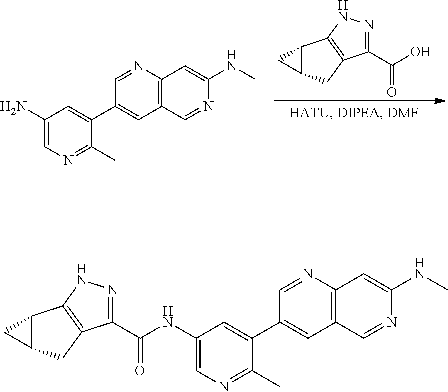

To a solution of (4aS,5aS)-4,4a,5,5a-tetrahydro-1H-cyclopropa[4,5]cyclopenta[1,2-c]pyrazole-3-carboxylic acid (30 mg, 0.18 mmol) in N,N-dimethylformamide (1 mL) were added N,N-diisopropylethylamine (35 mg, 0.27 mmol), 3-(5-amino-2-methylpyridin-3-yl)-N-methyl-1,6-naphthyridin-7-amine (48 mg, 0.18 mmol) and 1-[bis(dimethylamino)methylene]-1H-1,2,3-triazolo[4,5-b]pyridinium 3-oxid hexafluorophosphate (84 mg, 0.22 mmol) at room temperature. The reaction was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give (4aS,5aS)—N-(6-methyl-5-(7-(methylamino)-1,6-naphthyridin-3-yl)pyridin-3-yl)-4,4a,5,5a-tetrahydro-1H-cyclopropa[4,5]cyclopenta[1,2-c]pyrazole-3-carboxamide (8.9 mg, 0.02 mmol, 14%) as a yellow solid. ¹H NMR (400 MHz, DMSO-d₆) δ 12.97 (s, 1H), 9.89 (s, 1H), 8.97 (d, J=0.4 Hz, 1H), 8.86 (s, 1H), 8.81 (d, J=2.4 Hz, 1H), 8.25 (d, J=2.0 Hz, 1H), 8.12 (s, 1H), 6.74 (s, 1H), 6.65 (s, 1H), 2.90-2.89 (m, 4H), 2.78-2.73 (m, 1H), 2.49 (s, 3H), 2.19-2.17 (m, 2H), 1.16-1.11 (m, 1H), 0.27-0.26 (m, 1H). MS (ESI) m/z 412.1 [M+H]⁺

### Example 201. Synthesis of (4aR,5aR)—N-(6-methyl-5-(7-(methylamino)-1,6-naphthyridin-3-yl)pyridin-3-yl)-4,4a,5,5a-tetrahydro-1H-cyclopropa[4,5]cyclopenta[1,2-c]pyrazole-3-carboxamide (Compound 253)

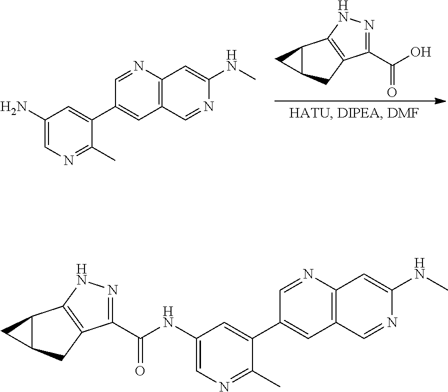

To a solution of (4aR,5aR)-4,4a,5,5a-tetrahydro-1H-cyclopropa[4,5]cyclopenta[1,2-c]pyrazole-3-carboxylic acid (30 mg, 0.18 mmol) in N,N-dimethylformamide (2 mL) were added N,N-diisopropylethylamine (0.1 mL), 3-(5-amino-2-methylpyridin-3-yl)-N-methyl-1,6-naphthyridin-7-amine (48 mg, 0.18 mmol) and 1-[bis(dimethylamino)methylene]-1H-1,2,3-triazolo[4,5-b]pyridinium 3-oxid hexafluorophosphate (137 mg, 0.36 mmol). The reaction was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give (4aR,5aR)—N-(6-methyl-5-(7-(methylamino)-1,6-naphthyridin-3-yl)pyridin-3-yl)-4,4a,5,5a-tetrahydro-1H-cyclopropa[4,5]cyclopenta[1,2-c]pyrazole-3-carboxamide (6.9 mg, 0.02 mmol, 9%) as a yellow solid. ¹H NMR (500 MHz, DMSO-d₆) δ 13.12 (s, 1H), 10.20 (s, 1H), 9.00 (s, 1H), 8.92 (s, 1H), 8.85 (d, J=2.3 Hz, 1H), 8.32 (d, J=1.8 Hz, 1H), 8.23 (s, 1H), 6.99 (s, 1H), 6.63 (s, 1H), 2.88 (d, J=3.8 Hz, 3H), 2.84-2.59 (m, 2H), 2.47 (s, 3H), 2.21 (s, 2H), 1.14 (td, J=7.8, 4.6 Hz, 1H), 0.28 (s, 1H). MS (ESI) m/z 412.2 [M+H]⁺

### Example 202. Synthesis of 5-isopropyl-N-(6-methyl-5-(7-(methylamino)-1,6-naphthyridin-3-yl)pyridin-3-yl)-4,5,6,7-tetrahydroisoxazolo[4,5-c]pyridine-3-carboxamide (Compound 254)

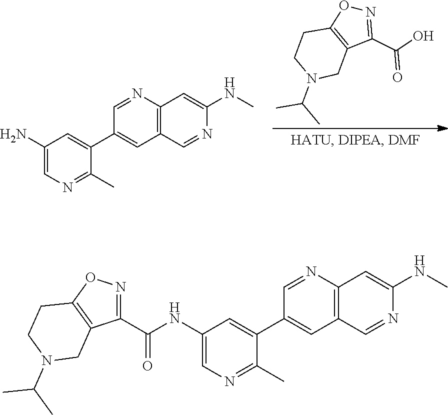

A mixture of 5-isopropyl-4,5,6,7-tetrahydroisoxazolo[4,5-c]pyridine-3-carboxylic acid (30 mg, 0.15 mmol), 3-(5-amino-2-methylpyridin-3-yl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (10 mL) was stirred at room temperature for 24 hours. The mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5-isopropyl-N-(6-methyl-5-(7-(methylamino)-1,6-naphthyridin-3-yl)pyridin-3-yl)-4,5,6,7-tetrahydroisoxazolo[4,5-c]pyridine-3-carboxamide (30 mg, 0.068 mmol, 45%). ¹H NMR (400 MHz, DMSO-d₆) 1H NMR δ 11.04 (s, 1H), 8.99 (s, 1H), 8.87 (dd, J=19.7, 2.3 Hz, 2H), 8.30 (d, J=2.0 Hz, 1H), 8.20 (d, J=2.3 Hz, 1H), 6.97 (d, J=4.9 Hz, 1H), 6.63 (s, 1H), 3.75 (s, 2H), 3.32 (d, J=9.4 Hz, 1H), 3.19-2.76 (m, 3H), 2.79 (d, J=20.4 Hz, 2H), 2.77 (dd, J=19.5, 14.3 Hz, 2H), 2.50 (dd, J=5.0, 3.3 Hz, 3H), 1.05 (d, J=6.5 Hz, 6H). MS (ESI) m/z 458 [M+H]⁺

### Example 203. Synthesis of 3-chloro-4-isopropyl-N-(6-methyl-5-(7-(methylamino)-1,6-naphthyridin-3-yl)pyridin-3-yl)picolinamide (Compound 256)

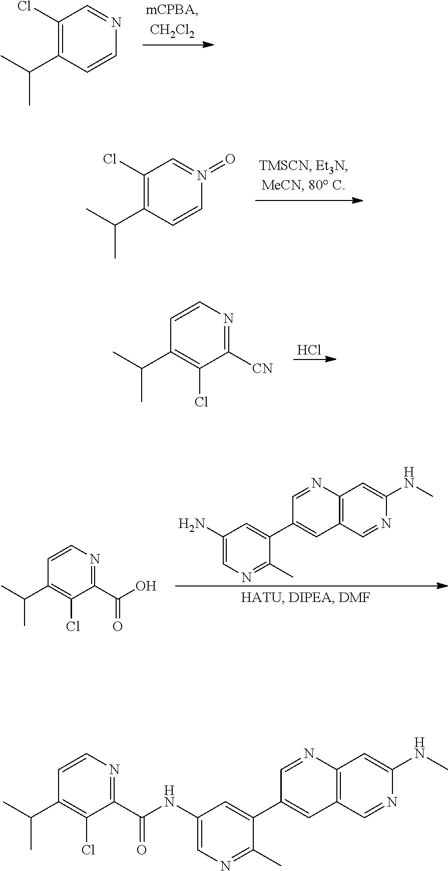

Step 1. A mixture of 3-chloro-4-isopropylpyridine (0.7 g, 4.5 mmol) and 3-chlorobenzoperoxoic acid (0.85 g, 5.0 mmol) in dichloromethane (10 mL) was stirred at 20° C. for 3 hours. The reaction mixture was treated with water (30 mL) and extracted with ethyl acetate (40 mL×3). The combined organic phases were concentrated. The residue was purified by flash chromatography (silica, methanol/dichloromethane=1/7) to give 3-chloro-4-isopropylpyridine 1-oxide (0.24 g, 1.4 mmol, 30%) as a yellow oil. MS (ESI) m/z 172.0 [M+H]⁺

Step 2. A mixture of 3-chloro-4-isopropylpyridine 1-oxide (0.24 g, 1.4 mmol), trimethylsilanecarbonitrile (554 mg, 5.6 mmol) and triethylamine (565 mg, 5.6 mmol) in acetonitrile (10 mL) was stirred at 80° C. for 16 hours. The reaction mixture was cooled to room temperature and concentrated. The residue was purified by flash chromatography (silica, petroleum ether/ethyl acetate=10/2) to give 3-chloro-4-isopropylpicolinonitrile (227 mg, 1.26 mmol, 90%) as a white solid. MS (ESI) m/z 181.0 [M+H]⁺

Step 3. A solution of 3-chloro-4-isopropylpicolinonitrile (227 mg, 1.26 mmol) in concentrated hydrochloric acid (10 mL) was stirred at 100° C. for 16 hours. The reaction mixture was cooled to room temperature and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-chloro-4-isopropylpicolinic acid (200 mg, 1.0 mmol, 80%) as a white solid. MS (ESI) m/z 200.1 [M+H]⁺

Step 4. A mixture of 3-chloro-4-isopropylpicolinic acid (40 mg, 0.2 mmol), 3-(5-amino-2-methylpyridin-3-yl)-N-methyl-1,6-naphthyridin-7-amine (52 mg, 0.2 mmol), 1-[bis(dimethylamino)methylene]-1H-1,2,3-triazolo[4,5-b]pyridinium 3-oxid hexafluorophosphate (152 mg, 0.4 mmol) and N,N-diisopropylethylamine (77 mg, 0.6 mmol) in N,N-dimethylformamide (5 mL) was stirred at room temperature for 2 hours. The reaction mixture was treated with water (30 mL) and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 3-chloro-4-isopropyl-N-(6-methyl-5-(7-(methylamino)-1,6-naphthyridin-3-yl)pyridin-3-yl)picolinamide (50 mg, 0.11 mmol, 56%) as a yellow solid. ¹H NMR (500 MHz, DMSO-d₆) δ 10.95 (s, 1H), 9.00 (s, 1H), 8.83 (dd, J=25.6, 2.3 Hz, 2H), 8.56 (d, J=5.0 Hz, 1H), 8.33 (d, J=2.1 Hz, 1H), 8.16 (d, J=2.3 Hz, 1H), 7.63 (d, J=5.0 Hz, 1H), 6.96 (q, J=4.9 Hz, 1H), 6.63 (s, 1H), 3.40 (dt, J=13.8, 6.9 Hz, 1H), 2.87 (d, J=4.9 Hz, 3H), 2.48 (s, 3H), 1.26 (d, J=6.9 Hz, 6H). MS (ESI) m/z 447.1 [M+H]⁺

### Example 204. Synthesis of 5,5-dimethyl-N-(6-methyl-5-(7-(methylamino)-1,6-naphthyridin-3-yl)pyridin-3-yl)-4,5,6,7-tetrahydrobenzo[d]isoxazole-3-carboxamide (Compound 257)

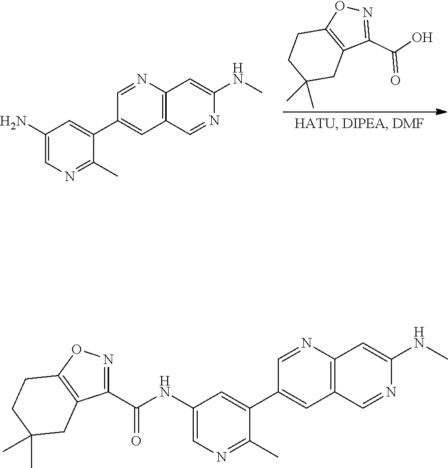

A solution of 5,5-dimethyl-4,5,6,7-tetrahydrobenzo[d]isoxazole-3-carboxylic acid (30 mg, 0.15 mmol), 3-(5-amino-2-methylpyridin-3-yl)-N-methyl-1,6-naphthyridin-7-amine (41 mg, 0.15 mmol), 1-[bis(dimethylamino)methylene]-1H-1,2,3-triazolo[4,5-b]pyridinium 3-oxid hexafluorophosphate (114 mg, 0.30 mmol) and N,N-diisopropylethylamine (58 mg, 0.45 mmol) in N,N-dimethylformamide (1 mL) was stirred at room temperature overnight. The solution was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5,5-dimethyl-N-(6-methyl-5-(7-(methylamino)-1,6-naphthyridin-3-yl)pyridin-3-yl)-4,5,6,7-tetrahydrobenzo[d]isoxazole-3-carboxamide (17.5 mg, 0.04 mmol, 26%) as a yellow solid. ¹H NMR (400 MHz, DMSO-d₆) δ 10.92 (s, 1H), 8.99 (s, 1H), 8.89 (d, J=1.6 Hz, 1H), 8.84 (d, J=2.0 Hz, 1H), 8.30 (s, 1H), 8.18 (d, J=1.6 Hz, 1H), 6.97 (d, J=4.0 Hz, 1H), 6.62 (s, 1H), 2.86 (d, J=3.6 Hz, 3H), 2.77 (t, J=5.2 Hz, 2H), 2.48 (s, 3H), 2.42 (s, 2H), 1.61 (t, J=5.2 Hz, 2H), 0.97 (s, 6H). MS (ESI) m/z 443.1 [M+H]⁺

### Example 205. Synthesis of (S)-6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide and (R)-6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide (Compounds 258 and 259)

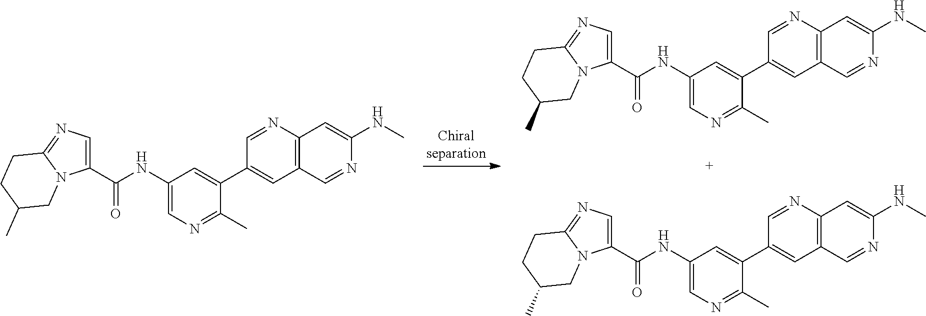 [SMILES: CNc1cc2ncc(-c3cc(NC(=O)c4cnc5n4CC(C)CC5)cnc3C)cc2cn1 ; CNc1cc2ncc(-c3cc(NC(=O)c4cnc5n4C[C@@H](C)CC5)cnc3C)cc2cn1 ; CNc1cc2ncc(-c3cc(NC(=O)c4cnc5n4C[C@H](C)CC5)cnc3C)cc2cn1]

The enantiomers of 6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide (40 mg, 0.094 mmol) were separated by chiral SFC (Instrument: SFC-150 (Waters); Column: OD 20×250 mm, 10 μm (Daicel); Mobile phase: CO₂/[MeOH/acetonitrile (0.2% NH₃ (7 M in MeOH)) 1:1]=45/55; Detection wavelength: 214 nm) to afford the first eluting enantiomer (retention time 3.6 min) which was arbitrarily assigned as (S)-6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide (8.6 mg, 0.020 mmol) as a yellow solid. ¹H NMR (500 MHz, DMSO-d₆) δ 1.04 (d, J=6.5 Hz, 3H), 1.46-1.53 (m, 1H), 1.89 (d, J=12.5 Hz, 1H), 2.02 (d, J=6.5 Hz, 1H), 2.46 (s, 3H), 2.72-2.81 (m, 1H), 2.86-2.90 (m, 4H), 3.61 (t, J=10.5 Hz, 1H), 4.51 (dd, J₁=5.0 Hz, J₂=13.5 Hz, 1H), 6.63 (s, 1H), 6.95 (q, J=4.5 Hz, 1H), 7.81 (s, 1H), 8.10 (d, J=2.5 Hz, 1H), 8.29 (d, J=2.0 Hz, 1H), 8.80 (d, J=3.0 Hz, 1H), 8.84 (d, J=2.5 Hz, 1H), 8.99 (s, 1H), 10.15 (s, 1H). MS (ESI) m/z 428.2 [M+H]⁺;
and the second eluting enantiomer (retention time 4.4 min) which was arbitrarily assigned as (R)-6-methyl-N-(6-methyl-5-(7-(methylamino)-1,6-naphthyridin-3-yl)pyridin-3-yl)-5,6,7,8-tetrahydroimidazo[1,2-a]pyridine-3-carboxamide ((11.7 mg, 0.027 mmol) as a yellow solid. ¹H NMR (500 MHz, DMSO-d₆) δ 1.04 (d, J=6.5 Hz, 3H), 1.48-1.53 (m, 1H), 1.89 (d, J=12.0 Hz, 1H), 2.02 (d, J=7.0 Hz, 1H), 2.46 (s, 3H), 2.72-2.80 (m, 1H), 2.86-2.90 (m, 4H), 3.61 (t, J=12.0 Hz, 1H), 4.51 (dd, J=6.0 Hz, J₂=13.5 Hz, 1H), 6.63 (s, 1H), 6.94 (q, J=5.0 Hz, 1H), 7.82 (s, 1H), 8.10 (d, J=2.0 Hz, 1H), 8.29 (d, J=2.0 Hz, 1H), 8.80 (d, J=3.0 Hz, 1H), 8.84 (d, J=2.0 Hz, 1H), 8.99 (s, 1H), 10.15 (s, 1H). MS (ESI) m/z 428.2 [M+H]⁺

### Example 206. Synthesis of 4-(1-methoxypropyl)-N-(6-methyl-5-(7-(methylamino)-1,6-naphthyridin-3-yl)pyridin-3-yl)picolinamide (Compound 260)

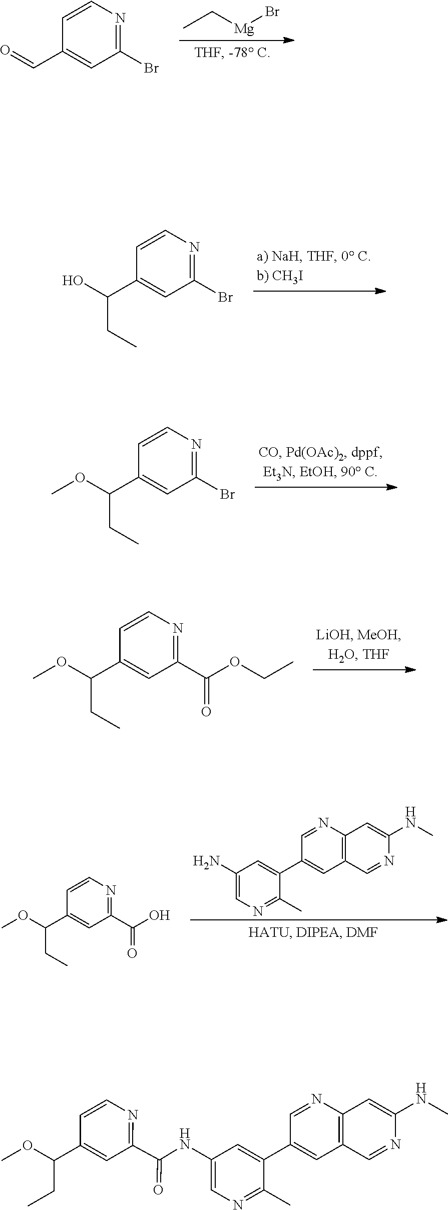

Step 1. A solution of 2-bromoisonicotinaldehyde (1000 mg, 5.38 mmol) in tetrahydrofuran (15 mL) was cooled to −78′° C. A solution of ethyl magnesium bromide (8.0 mL, 16.13 mmol, 2 M in tetrahydrofuran) was added dropwise. The reaction mixture was stirred for 1 hour at the same temperature. The reaction was quenched with saturated aqueous ammonium chloride solution (50 mL) and extracted with ethyl acetate (30 mL×3). The combined organic extracts were washed with brine (50 mL), dried over sodium sulfate, filtered, and concentrated in vacuo to give a crude product, which was purified by flash chromatography (silica, 0-50/a ethyl acetate in petroleum ether) to afford 1-(2-bromopyridin-4-yl)propan-1-ol (470 mg, 2.18 mmol, 41%) as a yellow solid. MS (ESI) m/z 218.2 [M+H]⁺

Step 2. To a solution of 1-(2-bromopyridin-4-yl)propan-1-ol (470 mg, 2.18 mmol) in tetrahydrofuran (6 mL) was added sodium hydride (261 mg, 6.53 mmol) at 0° C. and stirred for 10 minutes, and then iodomethane (371 mg, 2.61 mmol) was added. The resulting solution was stirred at room temperature for 2 hours. On completion, the reaction was quenched with water (20 mL) and extracted with ethyl acetate (50 mL×3). The organic layers were washed with saturated sodium bicarbonate aqueous solution (50 mL) and brine (40 mL), dried over sodium sulfate, filtered, and concentrated. The residue was purified by flash chromatography (silica, petroleum ether/ethyl acetate=1/1) to give 2-bromo-4-(1-methoxypropyl)pyridine (360 mg, 1.57 mmol, 72%₎ as a yellow solid. MS (ESI) m/z 230.2 [M+H]⁺

Step 3. A solution of 2-bromo-4-(1-methoxypropyl)pyridine (360 mg, 1.57 mmol), 1,1′-bis(diphenylphosphino)ferrocene (174 mg, 0.31 mmol), palladium (II) acetate (70 mg, 0.31 mmol) and triethylamine (476 mg, 4.72 mmol) in ethanol (8 mL) was stirred at 90° C. for 16 hours under carbon monoxide atmosphere. On completion, the reaction mixture was cooled to room temperature. The reaction mixture concentrated in vacuo to give a crude product. The residue was purified by flash chromatography (silica, petroleum ether/ethyl acetate=5/4) to afford ethyl 4-(1-methoxypropyl)picolinate (300 mg, 1.35 mmol, 86%) as a yellow solid. MS (ESI) m/z 224.2 [M+H]⁺

Step 4. Lithium hydroxide monohydrate (170 mg, 4.04 mmol) was added to a solution of ethyl 4-(1-methoxypropyl)picolinate (300 mg, 1.35 mmol) in tetrahydrofuran (3 mL), methanol (3 mL) and water (3 mL) at 0° C. The reaction mixture was stirred at 25° C. for 3 hours. The reaction solution was diluted with water (20 mL) and concentrated. The aqueous layer was acidified to pH=3 with 4 N hydrogen chloride and extracted with ethyl acetate (30 mL×2). The combined organic layers were washed with water (20 mL) and brine (20 mL), dried over sodium sulfate, filtered, and concentrated. The mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(1-methoxypropyl)picolinic acid (80 mg, 0.41 mmol, 31%) as a white solid. MS (ESI) m/z 196.1 [M+H]⁺

Step 5. To a solution of 4-(1-methoxypropyl)picolinic acid (80 mg, 0.41 mmol) in N,N-dimethylformamide (3 mL) were added N,N-diisopropylethylamine (159 mg, 1.23 mmol), 1-[bis(dimethylamino)methylene]-1H-1,2,3-triazolo[4,5-b]pyridinium 3-oxid hexafluorophosphate (234 mg, 0.62 mmol) and 3-(5-amino-2-methylpyridin-3-yl)-N-methyl-1,6-naphthyridin-7-amine (109 mg, 0.41 mmol). The mixture was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 4-(1-methoxypropyl)-N-(6-methyl-5-(7-(methylamino)-1,6-naphthyridin-3-yl)pyridin-3-yl)picolinamide (32.3 mg, 0.073 mmol, 18%) as a yellow solid. ¹H NMR (500 MHz, DMSO-d₆) δ 0.82 (t, J=7.0 Hz, 3H), 1.63-1.76 (m, 2H), 2.50 (s, 3H), 2.87 (d, J=5.0 Hz, 3H), 3.20 (s, 3H), 4.33 (t, J=6.5 Hz, 1H), 6.33 (s, 1H), 6.96 (d, J=5.5 Hz, 1H), 7.60 (d, J=5.5 Hz, 1H), 8.08 (s, 1H), 8.31 (t, J=5.5 Hz, 2H), 8.73 (d, J=5.0 Hz, 1H), 8.87 (d, J=2.0 Hz, 1H), 8.99 (s, 1H), 9.05 (d, J=1.5 Hz, 1H), 10.97 (s, 1H). MS (ESI) m/z 443.2 [M+H]⁺

### Example 207. Synthesis of 5-methyl-N-(4-methyl-3-(7-(methylamino)-1,6-naphthyridin-3-yl)phenyl)-4,5,6,7-tetrahydro-11H-indazole-3-carboxamide (Compound 261)

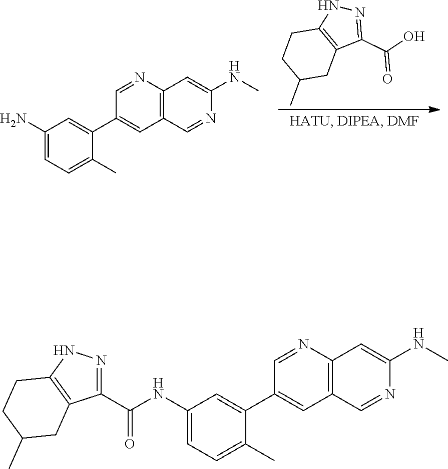

A mixture of 5-methyl-4,5,6,7-tetrahydro-1H-indazole-3-carboxylic acid (36 mg, 0.2 mmol), 3-(5-amino-2-methylpyridin-3-yl)-N-methyl-1,6-naphthyridin-7-amine (53 mg, 0.2 mmol), 1-[bis(dimethylamino)methylene]-1H-1,2,3-triazolo[4,5-b]pyridinium 3-oxid hexafluorophosphate (152 mg, 0.4 mmol) and N,N-diisopropylethylamine (77 mg, 0.6 mmol) in N,N-dimethylformamide (5 mL) was stirred at room temperature for 2 hours. The reaction mixture was treated with water (30 mL) and extracted with ethyl acetate (30 mL×2). The combined organic phases were concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5-methyl-N-(4-methyl-3-(7-(methylamino)-1,6-naphthyridin-3-yl)phenyl)-4,5,6,7-tetrahydro-1H-indazole-3-carboxamide (14 mg, 0.03 mmol, 16%) as a yellow solid. ¹H NMR (500 MHz, DMSO-d₆) δ 12.92 (s, 11H), 9.88 (s, 1H), 8.98 (s, 1H), 8.80 (d, J=2.2 Hz, 1H), 8.22 (d, J=1.7 Hz, 1H), 7.87 (s, 1H), 7.76 (d, J=8.3 Hz, 1H), 7.28 (d, J=8.3 Hz, 1H), 6.89 (d, J=4.9 Hz, 1H), 6.62 (s, 1H), 2.91 (dd, J=16.4, 4.7 Hz, 1H), 2.87 (d, J=4.8 Hz, 3H), 2.70 (d, J=14.6 Hz, 1H), 2.62 (m, J=16.0 Hz, 1H), 2.26 (s, 3H), 2.16 (dd, J=16.2, 9.8 Hz, 1H), 1.88-1.67 (m, 2H), 1.44-1.32 (m, 1H), 1.03 (d, J=6.6 Hz, 3H). MS (ESI) m/z 427.2 [M+H]⁺

### Example 208. Synthesis of 7,7-dimethyl-N-(4-methyl-3-(7-(methylamino)-1,6-naphthyridin-3-yl)phenyl)-4,5,6,7-tetrahydro-1H-indazole-3-carboxamide (Compound 262)

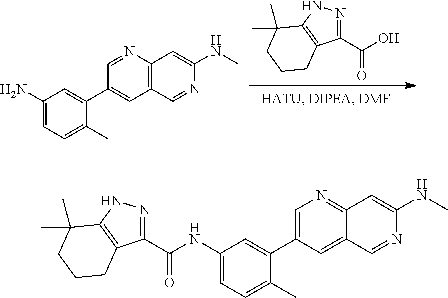

A mixture of 7,7-dimethyl-4,5,6,7-tetrahydro-1H-indazole-3-carboxylic acid (30 mg, 0.15 mmol), 3-(5-amino-2-methylphenyl)-N-methyl-1,6-naphthyridin-7-amine (50 mg, 0.18 mmol), 1-[bis(dimethylamino)methylene]-1H-1,2,3-triazolo[4,5-b]pyridinium 3-oxid hexafluorophosphate (68 mg, 0.18 mmol), and N,N-diisopropylethylamine (67 mg, 0.54 mmol) in N,N-dimethylformamide (2 mL) was stirred at room temperature for 1 hour. The mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 7,7-dimethyl-N-(4-methyl-3-(7-(methylamino)-1,6-naphthyridin-3-yl)phenyl)-4,5,6,7-tetrahydro-1H-indazole-3-carboxamide (30 mg, 0.068 mmol, 45%). ¹H NMR (400 MHz, DMSO-d₆) δ 12.98 (s, 1H), 9.85 (s, 1H), 8.98 (s, 1H), 8.80 (d, J=2.1 Hz, 1H), 8.22 (s, 1H), 7.83 (s, 1H), 7.76 (d, J=7.8 Hz, 1H), 7.28 (d, J=8.4 Hz, 1H), 6.89 (d, J=5.0 Hz, 1H), 6.62 (s, 1H), 2.87 (d, J=4.9 Hz, 3H), 2.64 (t, J=5.9 Hz, 2H), 2.26 (s, 3H), 1.70 (d, J=5.2 Hz, 2H), 1.57 (d, J=4.2 Hz, 2H), 1.25 (d, J=11.5 Hz, 6H). MS (ESI) m/z 441 [M+H]⁺

### Example 209. Synthesis of 5-methyl-N-(4-methyl-3-(7-(methylamino)-1,6-naphthyridin-3-yl)phenyl)-1H-indole-3-carboxamide (Compound 263)

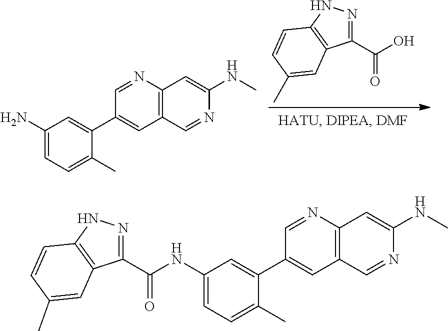

To a solution of 5-methyl-1H-indole-3-carboxylic acid (250 mg, 1.43 mmol) in N,N-dimethylformamide (8 mL) were added N,N-diisopropylethylamine (374 mg, 2.86 mmol), 1-[bis(dimethylamino)methylene]-1H-1,2,3-triazolo[4,5-b]pyridinium 3-oxid hexafluorophosphate (536 mg, 1.43 mmol) and 3-(5-amino-2-methylphenyl)-N-methyl-1,6-naphthyridin-7-amine (377 mg, 1.43 mmol). The mixture was stirred at room temperature for 12 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 5-methyl-N-(4-methyl-3-(7-(methylamino)-1,6-naphthyridin-3-yl)phenyl)-1H-indole-3-carboxamide (12.2 mg, 0.029 mmol, 2%) as a yellow solid. ¹H NMR (400 MHz, DMSO-d₆) δ 11.62 (s, 1H), 9.72 (s, 1H), 8.99 (s, 1H), 8.85 (d, J=2.4 Hz, 1H), 8.24 (d, J=2.8 Hz, 1H), 7.99 (s, 1H), 7.79-7.73 (m, 2H), 7.34-7.29 (m, 2H), 7.01-6.99 (m, 1H), 6.91-6.90 (m, 1H), 6.63 (s, 1H), 2.87 (d, J=5.2 Hz, 3H), 2.39 (s, 3H), 2.27 (s, 3H). MS (ESI) m/z 422.2 [M+H]⁺

### Example 210. Synthesis of (5S′,7R)-5,7-dimethyl-N-(4-methyl-3-(7-(methylamino)-1,6-naphthyridin-3-yl)phenyl)-1,4,5,7-tetrahydropyrano[3,4-c]pyrazole-3-carboxamide (Compound 264)

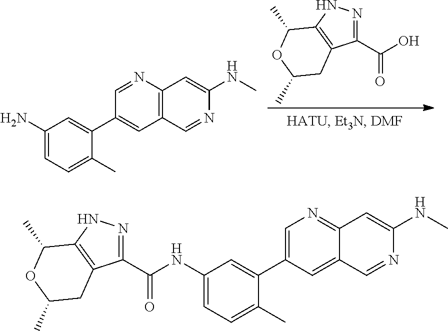

A solution of (5S,7R)-5,7-dimethyl-1,4,5,7-tetrahydropyrano[3,4-c]pyrazole-3-carboxylic acid (30 mg, 0.15 mmol), 3-(5-amino-2-methylphenyl)-N-methyl-1,6-naphthyridin-7-amine (40 mg, 0.15 mmol), 1-[bis(dimethylamino)methylene]-1H-1,2,3-triazolo[4,5-b]pyridinium 3-oxid hexafluorophosphate (114 mg, 0.30 mmol) and triethylamine (0.1 mL) in N,N-dimethylformamide (3 mL) was stirred at room temperature for 2 hours. The resulting mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford (5S,7R)-5,7-dimethyl-N-(4-methyl-3-(7-(methylamino)-1,6-naphthyridin-3-yl)phenyl)-1,4,5,7-tetrahydropyrano[3,4-c]pyrazole-3-carboxamide (4.9 mg, 0.01 mmol, 7%) as a yellow solid. ¹H NMR (400 MHz, DMSO-d₆) δ 13.15 (s, 1H), 9.98 (s, 1H), 8.98 (s, 1H), 8.80 (d, J=2.3 Hz, 1H), 8.22 (d, J=2.0 Hz, 1H), 7.86 (s, 1H), 7.77 (d, J=8.3 Hz, 1H), 7.29 (d, J=8.4 Hz, 1H), 6.90 (d, J=5.0 Hz, 1H), 6.62 (s, 1H), 4.80 (d, J=6.6 Hz, 1H), 3.71 (s, 1H), 2.87 (d, J=4.9 Hz, 3H), 2.37 (dd, J=25.4, 9.0 Hz, 2H), 2.27 (s, 3H), 1.45 (d, J=6.4 Hz, 3H), 1.27 (d, J=6.1 Hz, 3H). MS (ESI) m/z 443.1 [M+H]⁺

### Example 211. Synthesis of 5-methyl-N-(4-methyl-3-(7-(methylamino)-1,6-naphthyridin-3-yl)phenyl)-4,5,6,7-tetrahydropyrazolo[1,5-a]pyridine-3-carboxamide (Compound 265)

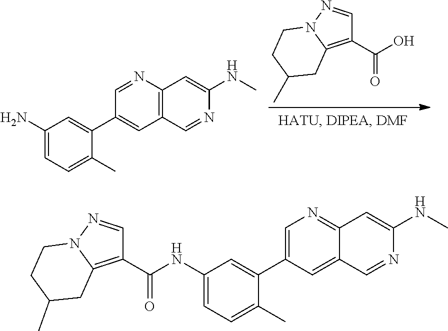

To a solution of 5-methyl-4,5,6,7-tetrahydropyrazolo[1,5-a]pyridine-3-carboxylic acid (30 mg, 0.17 mmol) in N,N-dimethylformamide (3 mL) were added N,N-diisopropylethylamine (64.5 mg, 0.3 mmol), 1-[bis(dimethylamino)methylene]-1H-1,2,3-triazolo[4,5-b]pyridinium 3-oxid hexafluorophosphate (95 mg, 0.25 mmol) and 3-(5-amino-2-methylphenyl)-N-methyl-1,6-naphthyridin-7-amine (30.8 mg, 0.12 mmol). The mixture was stirred at room temperature for 1 hour. The reaction was diluted with ethyl acetate (40 mL×3), washed with saturated sodium bicarbonate aqueous solution (50 mL) and brine (40 mL), dried over sodium sulfate, filtered, and concentrated. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 5-methyl-N-(4-methyl-3-(7-(methylamino)-1,6-naphthyridin-3-yl)phenyl)-4,5,6,7-tetrahydropyrazolo[1,5-a]pyridine-3-carboxamide (11.8 mg, 0.028 mmol, 16%) as a yellow solid. ¹H NMR (500 MHz, DMSO-d₆) δ 9.69 (s, 1H), 8.98 (s, 1H), 8.80 (d, J=2.2 Hz, 1H), 8.22 (d, J=1.9 Hz, 1H), 8.12 (s, 1H), 7.72 (s, 1H), 7.70-7.62 (m, 1H), 7.29 (d, J=8.4 Hz, 1H), 6.89 (d, J=5.0 Hz, 1H), 6.62 (s, 1H), 4.19 (d, J=12.8 Hz, 1H), 4.10-3.95 (m, 1H), 3.27 (d, J=5.1 Hz, 1H), 2.87 (d, J=4.8 Hz, 3H), 2.47-2.41 (m, 1H), 2.26 (s, 3H), 2.02-1.92 (m, 2H), 1.72-1.62 (m, 1H), 1.08 (d, J=6.5 Hz, 3H). MS (ESI) m/% z 427.2 [M+H]⁺

### Example 212. Synthesis of 5-methyl-N-(4-methyl-3-(7-(methylamino)-1,6-naphthyridin-3-yl)phenyl)-4,5,6,7-tetrahydro-1H-indole-3-carboxamide (Compound 266)

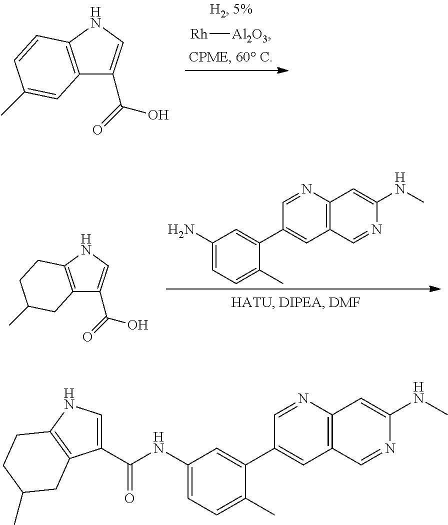

Step 1. To a solution of 5-methyl-1H-indole-3-carboxylic acid (500 mg, 2.86 mmol) in cyclopentyl methyl ether (10 mL) at 60° C. was added rhodium-aluminum oxide (5%, 500 mg). The reaction mixture was stirred at 60° C. for 72 hours under hydrogen atmosphere. After cooling to room temperature, the reaction was filtered through celite. The filtrate was concentrated in vacuo. The residue was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to give 5-methyl-4,5,6,7-tetrahydro-1H-indole-3-carboxylic acid (170 mg, 0.95 mmol, 33%) as a white solid. MS (ESI) m/z 180.1 [M+H]⁺

Step 2. To a solution of 5-methyl-4,5,6,7-tetrahydro-1H-indole-3-carboxylic acid (170 mg 0.95 mmol) in N,N-dimethylformamide (4 mL) were added N,N-diisopropylethylamine (248 mg, 1.90 mmol), 1-[bis(dimethylamino)methylene]-1H-1,2,3-triazolo[4,5-b]pyridinium 3-oxid hexafluorophosphate (356 mg, 0.95 mmol) and 3-(5-amino-2-methylphenyl)-N-methyl-1,6-naphthyridin-7-amine (251 mg, 0.95 mmol). The mixture was stirred at room temperature for 12 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 5-methyl-N-(4-methyl-3-(7-(methylamino)-1,6-naphthyridin-3-yl)phenyl)-4,5,6,7-tetrahydro-1H-indole-3-carboxamide (4 mg, 0.0094 mmol, 1%) as a yellow solid. ¹H NMR (400 MHz, DMSO-d₆) δ 10.85 (s, 1H), 9.32 (s, 1H), 8.98 (s, 1H), 8.80 (s, 1H), 8.21 (s, 1H), 7.73 (d, J=1.2 Hz, 1H), 7.67-7.65 (m, 1H), 7.26 (d, J=8.4 Hz, 1H), 6.90 (s, 1H), 6.62 (s, 1H), 2.92 (s, 1H), 2.87 (d, J=8.8 Hz, 3H), 2.18 (s, 3H), 2.15-2.11 (m, 2H), 1.80-1.77 (m, 2H), 1.33-1.23 (m, 2H), 1.02-1.00 (d, J=8.4 Hz, 3H). MS (ESI) m/z 426.2 [M+H]⁺

### Example 213. Synthesis of (R)—N-(4-methyl-3-(7-(methylamino)-1,6-naphthyridin-3-yl)phenyl)-3-(2,2,2-trifluoroethyl)pyrrolidine-1-carboxamide and (S)—N-(4-methyl-3-(7-(methylamino)-1,6-naphthyridin-3-yl)phenyl)-3-(2,2,2-trifluoroethyl)pyrrolidine-1-carboxamide (Compounds 267 and 268)

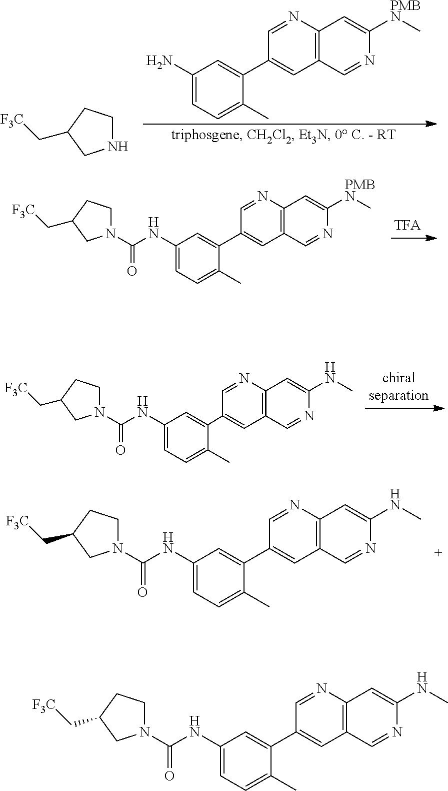

Step 1. To a solution of 3-(2,2,2-trifluoroethyl)pyrrolidine (100 mg, 0.65 mmol) in dichloromethane (5 ml) were added triethylamine (0.2 mL) and triphosgene (193 mg, 0.65 mmol) at 0° C. The mixture was stirred for 30 minutes at 0° C. 3-(5-Amino-2-methylphenyl)-N-(4-methoxybenzyl)-N-methyl-1,6-naphthyridin-7-amine (250 mg, 0.65 mmol) was added, and the mixture was stirred for another 4 hours at room temperature. The reaction was quenched with ice water (10 mL) and extracted with ethyl acetate (30 mL×3). The combined organic layers were dried over sodium sulfate, filtered, and concentrated in vacuo. The residue was purified by flash chromatography (silica, petroleum ether) to give N-(3-(7-((4-methoxybenzyl)(methyl)amino)-1,6-naphthyridin-3-yl)-4-methylphenyl)-3-(2,2,2-trifluoroethyl)pyrrolidine-1-carboxamide (245 mg, 0.43 mmol, 67%) as a yellow solid. MS (ESI) m/z 564.2 [M+H]⁺

Step 2. A mixture of N-(3-(7-((4-methoxybenzyl)(methyl)amino)-1,6-naphthyridin-3-yl)-4-methylphenyl)-3-(2,2,2-trifluoroethyl)pyrrolidine-1-carboxamide (245 mg, 0.43 mmol) in trifluoroacetic acid (5 mL) was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(4-methyl-3-(7-(methylamino)-1,6-naphthyridin-3-yl)phenyl)-3-(2,2,2-trifluoroethyl)pyrrolidine-1-carboxamide (80 mg, 0.18 mmol, 42%) as a yellow solid. MS (EST) m/z 444.1 [M+H]⁺

Step 3. The enantiomers of N-(4-methyl-3-(7-(methylamino)-1,6-naphthyridin-3-yl)phenyl)-3-(2,2,2-trifluoroethyl)pyrrolidine-1-carboxamide (80 mg, 0.18 mmol) were separated by chiral SFC (Instrument: SFC-150 (Waters); Column: OZ 20×250 mm, 10 μm (Daicel); Mobile phase: CO₂/MeOH (0.2% NH₃ (7 M in MeOH))=55/45; Detection wavelength: 214 nm) to afford the first eluting enantiomer (retention time 2.4 min) which was arbitrarily assigned as (R)—N-(4-methyl-3-(7-(methylamino)-1,6-naphthyridin-3-yl)phenyl)-3-(2,2,2-trifluoroethyl)pyrrolidine-1-carboxamide (6.8 mg, 0.015 mmol) as a yellow solid. ¹H NMR (500 MHz, CDCl₃) δ 8.85-8.82 (m, 2H), 7.99 (d, J=1.8 Hz, 1H), 7.41 (d, J=2.3 Hz, 1H), 7.34 (dd, J=8.2, 2.3 Hz, 1H), 7.23 (d, J=8.3 Hz, 1H), 6.78 (s, 1H), 6.27 (s, 1H), 5.13 (s, 1H), 3.84-3.77 (m, 1H), 3.64 (t, J=8.1 Hz, 1H), 3.44 (td, J=9.7, 6.8 Hz, 1H), 3.12 (t, J=9.5 Hz, 1H), 3.02 (s, 3H), 2.59-2.51 (m, 1H), 2.27-2.23 (m, 6H), 1.78-1.69 (m, 1H). MS (ESI) m/z 444.1 [M+H]⁺;
and the second eluting enantiomer (retention time 3.1 min) which was arbitrarily assigned as (S)—N-(4-methyl-3-(7-(methylamino)-1,6-naphthyridin-3-yl)phenyl)-3-(2,2,2-trifluoroethyl)pyrrolidine-1-carboxamide (5.0 mg, 0.011 mmol) as a yellow solid. ¹H NMR (500 MHz, CDCl₃) δ 8.85-8.83 (m, 2H), 7.98 (d, J=1.8 Hz, 1H), 7.41 (d, J=2.1 Hz, 1H), 7.34 (dd, J=8.2, 2.2 Hz, 1H), 7.24 (d, J=8.3 Hz, 1H), 6.77 (s, 1H), 6.19 (s, 1H), 4.96 (d, J=5.1 Hz, 1H), 3.84-3.78 (m, 1H), 3.64 (t, J=8.1 Hz, 1H), 3.45 (td, J=9.7, 6.9 Hz, 1H), 3.12 (t, J=9.4 Hz, 1H), 3.03 (d, J=5.3 Hz, 3H), 2.56 (d, J=7.2 Hz, 1H), 2.29-2.23 (m, 6H), 1.78-1.72 (m, 1H). MS (ESI) m/z 444.1 [M+H]⁺

### Example 214. Synthesis of N-[3-[8-chloro-7-(methylamino)-1,6-naphthyridin-3-yl]-4-methyl-phenyl]-2-(1-fluorocyclopropyl)pyridine-4-carboxamide (Compound 269)

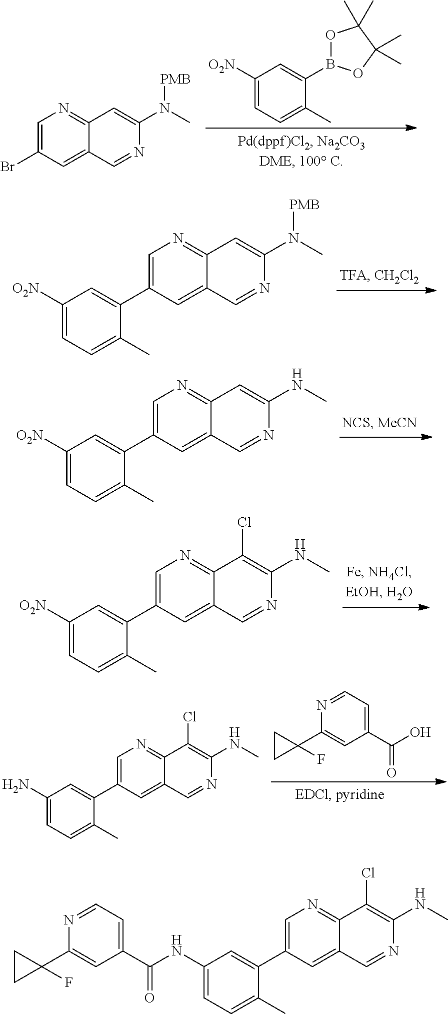

Step 1. To a solution of 3-bromo-N-[(4-methoxyphenyl)methyl]-N-methyl-1,6-naphthyridin-7-amine (2 g, 5.58 mmol) in dimethoxyethane (15 mL) were added 4,4,5,5-tetramethyl-2-(2-methyl-5-nitro-phenyl)-1,3,2-dioxaborolane (4.41 g, 16.7 mmol), 1,1′-bis(diphenylphosphino) ferrocene-palladium(II) dichloride (408 mg, 558 μmol) and sodium carbonate (1.18 g, 11.2 mmol). The mixture was stirred at 100° C. for 12 hours under nitrogen atmosphere. The mixture was concentrated under reduced pressure. The residue was purified by flash chromatography (silica, petroleum ether/ethyl acetate=20/1 to 1/1) to give N-[(4-methoxyphenyl)methyl]-N-methyl-3-(2-methyl-5-nitro-phenyl)-1,6-naphthyridin-7-amine (1.1 g, 2.63 mmol, 47%) as a white solid. MS (ESI) m/z 415.3 [M+H]⁺

Step 2. To a solution of N-[(4-methoxyphenyl)methyl]-N-methyl-3-(2-methyl-5-nitro-phenyl)-1,6-naphthyridin-7-amine (1.1 g, 2.65 mmol) in dichloromethane (10 mL) was added trifluoroacetic acid (7.70 g, 67.5 mmol). The mixture was stirred at 25° C. for 2 hours. The mixture was concentrated under reduced pressure. The residue was purified by reversed-phase HPLC (0.1% formic acid) to give N-methyl-3-(2-methyl-5-nitro-phenyl)-1,6-naphthyridin-7-amine (0.9 g, 2.64 mmol, 99.6% formic acid salt) as a white solid. MS (ESI) m/z 295.4 [M+H]⁺

Step 3. To a solution of N-methyl-3-(2-methyl-5-nitro-phenyl)-1,6-naphthyridin-7-amine (0.7 g, 2.06 mmol formic acid salt) in acetonitrile (10 mL) was added N-chlorosuccinimide (302 mg, 2.26 mmol) in portions. The mixture was stirred at 25° C. for 12 hours. The mixture was concentrated under reduced pressure. The residue was purified by flash chromatography (silica, petroleum ether/ethyl acetate=20/1 to 1/1) to give 8-chloro-N-methyl-3-(2-methyl-5-nitro-phenyl)-1,6-naphthyridin-7-amine (290 mg, 882 μmol, 43%) as a white solid. MS (ESI) m/z 329.1 [M+H]⁺

Step 4. To a solution of 8-chloro-N-methyl-3-(2-methyl-5-nitro-phenyl)-1,6-naphthyridin-7-amine (340 mg, 1.03 mmol) in water (1 mL) and ethanol (5 mL) were added iron (288 mg, 5.17 mmol) and ammonium chloride (55 mg, 10.3 mmol). The mixture was stirred at 80° C. for 1 hour. The mixture was filtered, and the filtrate was concentrated under reduced pressure. The residue was purified by flash chromatography (silica, petroleum ether/ethyl acetate=20/1 to 1/1) to give 3-(5-amino-2-methyl-phenyl)-8-chloro-N-methyl-1,6-naphthyridin-7-amine (190 mg, 635 μmol, 61.5%) as a white solid. MS (ESI) m/z 299.4 [M+H]⁺

Step 5. To a solution of 2-(1-fluorocyclopropyl)pyridine-4-carboxylic acid (109 mg, 602 μmol) in pyridine (2 mL) was added 1-ethyl-3-(3-dimethylaminopropyl)carbodiimide (173 mg, 903 μmol) at 0° C. and then 3-(5-amino-2-methyl-phenyl)-8-chloro-N-methyl-1,6-naphthyridin-7-amine (180 mg, 602 μmol) was added. The mixture was stirred at 25° C. for 1 hour. The mixture was concentrated under reduced pressure. The residue was purified by prep-HPLC (Column: Unisil 3-100 C18 Ultra 50×150 mm, 3 μm, Mobile Phase: A: water (formic acid), B: acetonitrile; B %: 40%-70% in 10 min) to give N-[3-[8-chloro-7-(methylamino)-1,6-naphthyridin-3-yl]-4-methyl-phenyl]-2-(1-fluorocyclopropyl)pyridine-4-carboxamide (25.4 mg, 54.5 μmol, 9%) as a yellow solid. ¹H NMR (400 MHz, DMSO-6) δ 10.65 (s, 1H), 9.07 (s, 1H), 8.98 (d, J=2.4 Hz, 1H), 8.71 (d, J=5.2 Hz, 1H), 8.37 (d, J=2.4 Hz, 1H), 8.09 (s, 1H), 7.82-7.74 (m, 3H), 7.39 (d, J=8.8 Hz, 1H), 6.92-6.85 (m, 1H), 3.05 (d, J=4.8 Hz, 3H), 2.30 (s, 3H), 1.62 (d, J=2.9 Hz, 1H), 1.58-1.53 (m, 1H), 1.45-1.37 (m, 2H). MS (ESI) m/z 461.9 [M+H]⁺

### Example 215. Synthesis of N-[3-[8-cyano-7-(methylamino)-1,6-naphthyridin-3-yl]-4-methyl-phenyl]-2-(1-fluorocyclopropyl)pyridine-4-carboxamide (Compound 271)

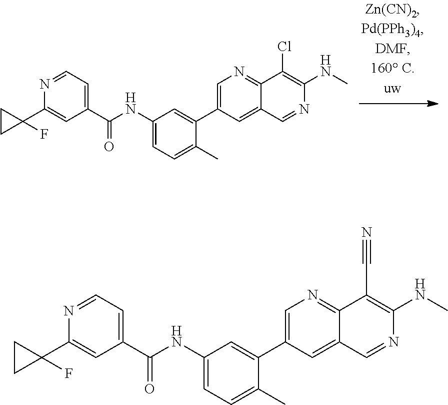 [SMILES: CNc1ncc2cc(-c3cc(NC(=O)c4ccnc(C5(F)CC5)c4)ccc3C)cnc2c1Cl ; CNc1ncc2cc(-c3cc(NC(=O)c4ccnc(C5(F)CC5)c4)ccc3C)cnc2c1C#N]

To a solution of N-[3-[8-chloro-7-(methylamino)-1,6-naphthyridin-3-yl]-4-methyl-phenyl]-2-(1-fluorocyclopropyl)pyridine-4-carboxamide (50 mg, 108 μmol) in N,N-dimethylformamide (1 mL) were added zinc cyanide (63.5 mg, 541 μmol) and tetrakis(triphenylphosphine)palladium(0) (12.5 mg, 10.8 μmol). The mixture was stirred at 160° C. for 1 hour under microwave irradiation under nitrogen atmosphere. The reaction mixture was quenched with water (10 mL) at 0° C., and then extracted with ethyl acetate (10 mL×3). The combined organic layers were washed with brine (10 mL×1), dried over sodium sulfate, filtered, and concentrated under reduced pressure. The residue was purified by prep-HPLC (Column: Unisil 3-100 C18 Ultra 50×150 mm, 3 μm, Mobile Phase: A: water (formic acid), B: acetonitrile; B %: 45%-60% in 10 min) and then re-purified by prep-HPLC (Column: Phenomenex Luna C18, 30×75 mm, 3 μm, Mobile Phase: A: water (hydrochloric acid), B: acetonitrile; B %: 49%-69%) to give N-[3-[8-cyano-7-(methylamino)-1,6-naphthyridin-3-yl]-4-methyl-phenyl]-2-(1-fluorocyclopropyl)pyridine-4-carboxamide (26.0 mg, 54.6 μmol, 50%) as a white solid. ¹H NMR (400 MHz, CD₃OD) δ 9.29 (s, 1H), 8.94 (d, J=2.0 Hz, 1H), 8.71 (d, J=5.2 Hz, 1H), 8.61 (d, J=2.0 Hz, 1H), 8.13 (s, 1H), 7.87-7.79 (m, 2H), 7.71 (J=2.4, 8.3 Hz, 1H), 7.47-7.39 (m, 1H), 3.23 (s, 3H), 2.37 (s, 3H), 1.68-1.59 (m, 2H), 1.57-1.48 (m, 2H). MS (ESI) m/z 453.0 [M+H]⁺

### Example 216. Synthesis of N-[5-[8-chloro-7-(methylamino)-1,6-naphthyridin-3-yl]-6-methyl-3-pyridyl]-2-(1-cyano-1-methyl-ethyl)pyridine-4-carboxamide (Compound 270)

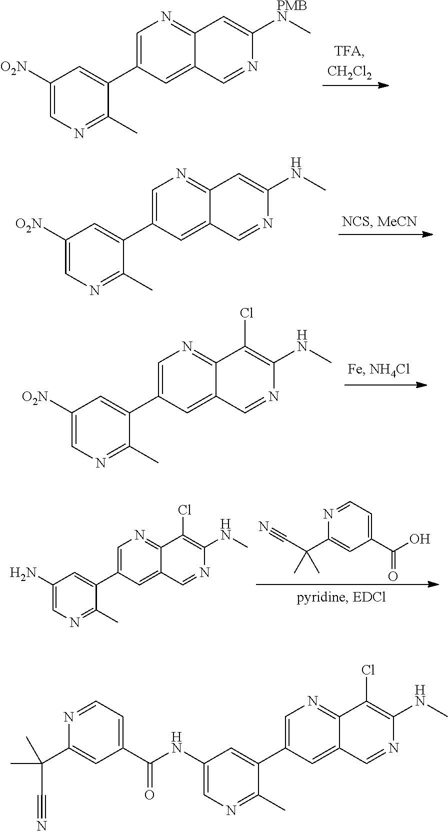

Step 1. To a solution of N-[(4-methoxyphenyl)methyl]-N-methyl-3-(2-methyl-5-nitro-1-pyridyl)-1,6-naphthyridin-7-amine (1.5 g, 3.61 mmol) in dichloromethane (15 mL) was added trifluoroacetic acid (7.70 g, 67.5 mmol). The mixture was stirred at 25° C. for 4 hours. The mixture was concentrated under reduced pressure. The residue was purified by reversed-phase HPLC (0.1% formic acid) to give N-methyl-3-(2-methyl-5-nitro-3-pyridyl)-1,6-naphthyridin-7-amine (1.2 g, 3.52 mmol, 97%, formic acid salt) as a white solid. MS (ESI) m/z 296.4 [M+H]⁺

Step 2. To a solution of N-methyl-3-(2-methyl-5-nitro-3-pyridyl)-1,6-naphthyridin-7-amine (0.27 g, 791 μmol formic acid salt) in acetonitrile (4 mL) was added N-chlorosuccinimide (105 mg, 791 μmol) at 0° C., and the mixture was stirred at 25° C. for 2 hours. The mixture was concentrated under reduced pressure. The residue was purified by flash chromatography (silica, petroleum ether/ethyl acetate=20/1 to 1/1) to give 8-chloro-N-methyl-3-(2-methyl-5-nitro-3-pyridyl)-1,6-naphthyridin-7-amine (130 mg, 354 μmol) as a white solid. MS (ESI) m/z 330.3 [M+H]⁺

Step 3. To a solution of 8-chloro-N-methyl-3-(2-methyl-5-nitro-3-pyridyl)-1,6-naphthyridin-7-amine (198 mg, 600 μmol) in ethanol (6 mL) and water (1 mL) was added iron (268 mg, 4.80 mmol) and ammonium chloride (481 mg, 9.01 mmol). The mixture was stirred at 80° C. for 2 hours. The mixture was filtered, and the filtrate was concentrated under reduced pressure. The residue was purified by flash chromatography (silica, petroleum ether/ethyl acetate=20/1 to 1/1) to give 3-(5-amino-2-methyl-3-pyridyl)-8-chloro-N-methyl-1,6-naphthyridin-7-amine (150 mg, 500 μmol, 83%) as a white solid. MS (EST) m/z 300.4 [M+H]⁺

Step 4. To a solution of 2-(1-cyano-1-methyl-ethyl)pyridine-4-carboxylic acid (28.6 mg, 150 μmol) in pyridine (0.5 mL) was added 1-ethyl-3-(3-dimethylaminopropyl)carbodiimide (38.4 mg, 200 μmol) and 3-(5-amino-2-methyl-3-pyridyl)-8-chloro-N-methyl-1,6-naphthyridin-7-amine (30 mg, 100 μmol). The mixture was stirred at 25° C. for 1 hour. The mixture was concentrated under reduced pressure. The residue was purified by prep-HPLC (Column: Unisil 3-100 C18 Ultra 50×150 mm, 3 μm, Mobile Phase: A: water (formic acid), B: acetonitrile; B %: 25%-50% in 10 min) to give N-[5-[8-chloro-7-(methylamino)-1,6-naphthyridin-3-yl]-6-methyl-3-pyridyl]-2-(1-cyano-1-methyl-ethyl)pyridine-4-carboxamide (6.95 mg, 14.7 μmol, 15%) as a yellow solid. ¹H NMR (400 MHz, DMSO-d₆) δ 11.04 (s, 1H), 9.07 (s, 2H), 9.03 (d, J=2.4 Hz, 1H), 8.83 (d, J=5.2 Hz, 1H), 8.45 (d, J=2.4 Hz, 1H), 8.35-8.29 (m, 2H), 7.91-7.84 (m, 1H), 7.02-6.89 (m, 1H), 3.36-3.33 (m, 75H), 3.05 (d, J=4.8 Hz, 3H), 2.48-2.43 (m, 3H), 1.77 (s, 6H). MS (ESI) m/z 472.0 [M+H]⁺

### Example 217. Synthesis of N-[5-[8-cyano-7-(methylamino)-1,6-naphthyridin-3-yl]-6-methyl-3-pyridyl]-2-(1-cyano-1-methyl-ethyl)pyridine-4-carboxamide (Compound 192)

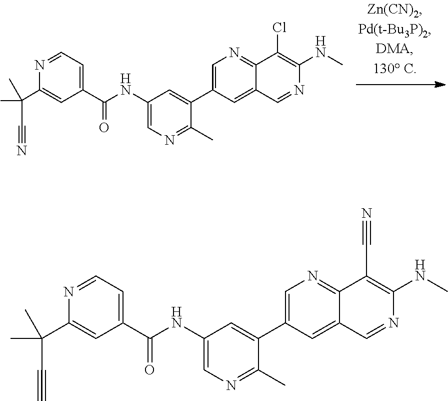

To a solution N-[5-[8-chloro-7-(methylamino)-1,6-naphthyridin-3-yl]-6-methyl-3-pyridyl]-2-(1-cyano-1-methyl-ethyl)pyridine-4-carboxamide (60 mg, 127 μmol) in N N-dimethylacetamide (1 mL) were added bis(tri-tert-butylphosphine)palladium(0) (13.0 mg, 25.4 μmol) and zinc cyanide (59.7 mg, 508 μmol). The mixture was stirred at 130° C. for 2 hours under nitrogen atmosphere. The reaction mixture was quenched with water (10 mL) at 0° C., and then extracted with ethyl acetate (10 mL×3). The combined organic layers were washed with brine (10 mL×1), dried over sodium sulfate, filtered, and concentrated under reduced pressure. The residue was purified by prep-HPLC (Column: Unisil 3-100 C18 Ultra 50×150 mm, 3 μm, Mobile Phase: A: water (formic acid), B: acetonitrile; B %: 25%-55% in 10 min) to give N-[5-[8-cyano-7-(methylamino)-1,6-naphthyridin-3-yl]-6-methyl-3-pyridyl]-2-(1-cyano-1-methyl-ethyl)pyridine-4-carboxamide (5.89 mg, 12.5 μmol, 10%) as a yellow solid. ¹H NMR (400 MHz, DMSO-d₆) δ 11.06 (s, 1H), 9.29 (s, 1H), 9.09-9.05 (m, 2H), 8.83 (d, J=5.2 Hz, 1H), 8.50 (d, J=2.0 Hz, 1H), 8.37-8.27 (m, 2H), 7.91-7.84 (m, 1H), 7.81-7.74 (m, 1H), 3.08 (d, J=4.4 Hz, 3H), 2.50-2.47 (m, 3H), 1.78 (s, 6H). MS (EST) m/z 463.0 [M+H]⁺

### Example 218. Synthesis of N-(3-(5-cyano-3-(methylamino)isoquinolin-7-yl)-4-methylphenyl)-2-(1-fluorocyclopropyl)isonicotinamide (Compound 272)

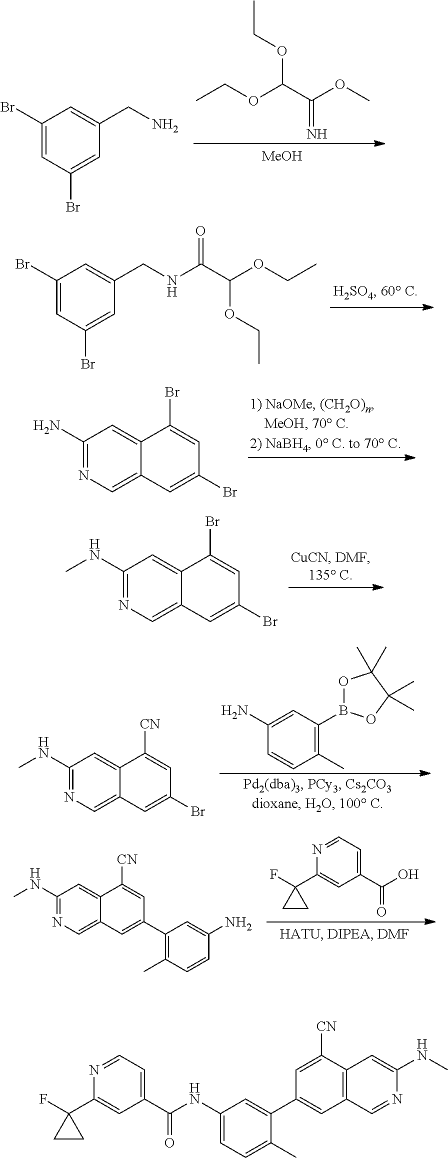

Step 1. Methyl 2,2-diethoxyacetimidate (500 mg, 3.1 mmol) was added to a mixture of (3,5-dibromophenyl) methanamine (400 mg, 1.51 mmol) in methanol (10 mL). The reaction mixture was stirred at 25° C. for 12 hours. The mixture was concentrated, and the residue was dissolved in dichloromethane (100 mL), washed with water (20 mL) and brine (20 mL), and dried over sodium sulfate. The organic phase was filtered and concentrated to give N-(3,5-dibromobenzyl)-2,2-diethoxyacetimidamide (500 mg, 1.26 mmol, 84%) as a brown solid which was used without further purification. MS (ESI) m/z 393.0, 395.0 [M+H]⁺

Step 2. A solution of N-(3,5-dibromobenzyl)-2,2-diethoxyacetimidamide (500 mg, 1.26 mmol) in sulfuric acid (5 mL) was stirred at 60° C. for 12 hours. After cooling to room temperature, the mixture was poured into ice-water (30 mL). The solution was basified with potassium carbonate until pH=10 and extracted with dichloromethane (50 mL×3). The combined organic layers were washed with brine (30 mL), dried over sodium sulfate, filtered, and concentrated to give 5,7-dibromoisoquinolin-3-amine (330 mg, 1.09 mmol, 96%) as a yellow solid which was used without further purification. MS (ESI) m/z 301.0, 303.0 [M+H]⁺

Step 3. Paraformaldehyde (328 mg, 10.9 mmol) was added to a solution of sodium methanolate (590 mg, 10.9 mmol) and 5,7-dibromoisoquinolin-3-amine (330 mg, 1.09 mmol) in methanol (15 mL). The mixture was stirred at 70° C. for 2 hours. After cooling to 0° C., sodium borohydride (413 mg, 10.9 mmol) was added to the mixture. The reaction solution was stirred at 70° C. for 2 hours. After cooling to room temperature, the mixture was poured into ice-water (60 mL) and extracted with dichloromethane (50 mL×3). The combined organic layers were washed with brine (30 mL), dried over sodium sulfate, filtered, and concentrated. The residue was purified by flash chromatography (silica, petroleum ether/ethyl acetate=7/1) to give 5,7-dibromo-N-methylisoquinolin-3-amine (150 mg, 0.47 mmol, 43%) as a yellow solid. MS (ESI) m/z 315.0, 317.0 [M+H]⁺

Step 4. A mixture of 5,7-dibromo-N-methylisoquinolin-3-amine (150 mg, 0.48 mmol) and copper(I) cyanide (85 mg, 0.96 mmol) in N,N-dimethylformamide (5 mL) was stirred at 135° C. overnight under nitrogen atmosphere. After cooling to room temperature, the mixture was poured into water (10 mL) and extracted with dichloromethane (30 mL×3). The combined organic layers were washed with brine (30 mL), dried over sodium sulfate, filtered, and concentrated. The residue was purified by flash chromatography (silica, petroleum ether/ethyl acetate=1/2) to give 7-bromo-3-(methylamino)isoquinoline-5-carbonitrile (130 mg, 0.50 mmol, 36%) as a yellow solid. MS (ESI) m/z 261.8, 263.7 [M+H]⁺

Step 5. To a solution of 7-bromo-3-(methylamino)isoquinoline-5-carbonitrile (130 mg, 0.50 mmol) in 1,4-dioxane (10 mL) and water (2 mL) were added 4-methyl-3-(4,4,5,5-tetramethyl-1,3,2-dioxaborolan-2-yl)aniline (174 mg, 0.75 mmol), cesium carbonate (33 mg, 0.1 mmol), tris(dibenzylideneacetone)dipalladium (69 mg, 0.075 mmol) and tricyclohexylphosphane (42 mg, 0.15 mmol). The reaction mixture was stirred at 100° C. for 16 hours under nitrogen. The mixture was diluted with water (10 mL) and extracted with ethyl acetate (30 mL×3). The combined organic layers were washed with water (50 mL), then brine (50 mL), dried over sodium sulfate, filtered, and concentrated. The residue was purified by prep-HPLC (Column: Xbridge 21.2×250 mm C18, 10 μm, Mobile Phase A: water (10 mM ammonium bicarbonate), B: acetonitrile) to give 7-(5-amino-2-methylphenyl)-3-(methylamino)isoquinoline-5-carbonitrile (12 mg, 0.04 mmol, 8%) as a yellow solid. MS (ESI) m/z 289.2 [M+H]⁺

Step 6. A mixture of 2-(1-fluorocyclopropyl)isonicotinic acid (8 mg, 0.04 mmol), 7-(5-amino-2-methylphenyl)-3-(methylamino)isoquinoline-5-carbonitrile (12 mg, 0.04 mmol), 1-[bis(dimethylamino)methylene]-1H-1,2,3-triazolo[4,5-b]pyridinium 3-oxid hexafluorophosphate (19 mg, 0.05 mmol) and N,N-diisopropylethylamine (10 mg, 0.08 mmol) in N,N-dimethylformamide (2 mL) was stirred at room temperature for 2 hours. The residue was purified by prep-HPLC (Column: Xbridge 21.2×250 mm C18, 10 μm, Mobile Phase A: water (10 mM ammonium bicarbonate), B: acetonitrile) to give N-(3-(5-cyano-3-(methylamino)isoquinolin-7-yl)-4-methylphenyl)-2-(1-fluorocyclopropyl)isonicotinamide (7.7 mg, 0.017 mmol, 43%) as a yellow solid. 1H NMR (400 MHz, DMSO-de) δ 10.63 (s, 1H), 9.11 (s, 1H), 8.71 (d, J=4.8 Hz, 1H), 8.20-8.17 (m, 2H), 8.09 (s, 1H), 7.78 (d, J=6.4 Hz, 3H), 7.37-7.35 (m, 1H), 7.22-7.20 (m, 1H), 6.6 (s, 1H), 2.90 (d, J=4.4 Hz, 3H), 2.29 (s, 3H), 1.64-1.55 (m, 2H), 1.44-1.38 (m, 2H). MS (ESI) m/z 452.4 [M+H]⁺

### Example 219. Synthesis of N-(3-(5-cyano-3-(methylamino)isoquinolin-7-yl)-4-methylphenyl)-2-(trifluoromethyl)isonicotinamide (Compound 273)

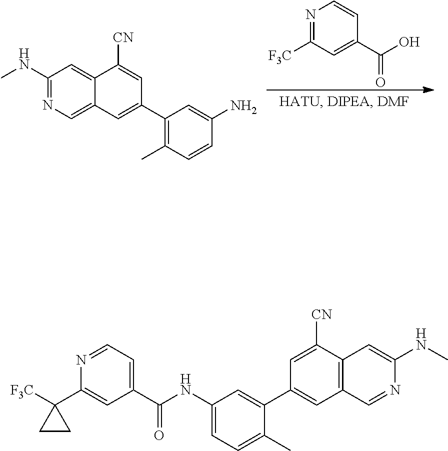 [SMILES: CNc1cc2c(C#N)cc(-c3cc(N)ccc3C)cc2cn1 ; O=C(O)c1ccnc(C(F)(F)F)c1 ; CNc1cc2c(C#N)cc(-c3cc(NC(=O)c4ccnc(C5(C(F)(F)F)CC5)c4)ccc3C)cc2cn1]

A mixture of 2-(trifluoromethyl)isonicotinic acid (18 mg, 0.09 mmol), 7-(5-amino-2-methylphenyl)-3-(methylamino)isoquinoline-5-carbonitrile (27 mg, 0.09 mmol), 1-[bis(dimethylamino)methylene]-1H-1,2,3-triazolo[4,5-b]pyridinium 3-oxid hexafluorophosphate (43 mg, 0.11 mmol) and N,N-diisopropylethylamine (24 mg, 0.19 mmol) in N,N-dimethylformamide (3 mL) was stirred at room temperature for 2 hours. The reaction mixture was concentrated. The residue was purified by prep-HPLC (Column: Xbridge 21.2×250 mm C18, 10 μm, Mobile Phase A: water (10 mM ammonium bicarbonate), B: acetonitrile) to give N-(3-(5-cyano-3-(methylamino)isoquinolin-7-yl)-4-methylphenyl)-2-(trifluoromethyl)isonicotinamide (6.3 mg, 0.013 mmol, 15%) as a yellow solid. ¹H NMR (400 MHz, CD₃OD) δ 9.00 (s, 1H), 8.93 (d, J=5.2 Hz, 1H), 8.33 (s, 1H), 8.15 (d, J=5.2 Hz, 1H), 8.12 (s, 1H), 8.05 (d, J=2.0 Hz, 1H), 7.73-7.69 (m, 2H), 7.39 (d, J=8.4 Hz, 1H), 6.76 (s, 1H), 3.03 (s, 3H), 2.34 (s, 3H). MS (ESI) m/z 462.3 [M+H]⁺

### Example 220. Synthesis of N-(6-methyl-5-(2-oxo-2,3-dihydro-1H-pyrrolo[2,3-c]isoquinolin-7-yl)pyridin-3-yl)-4-(trifluoromethyl)picolinamide (Compound 274)

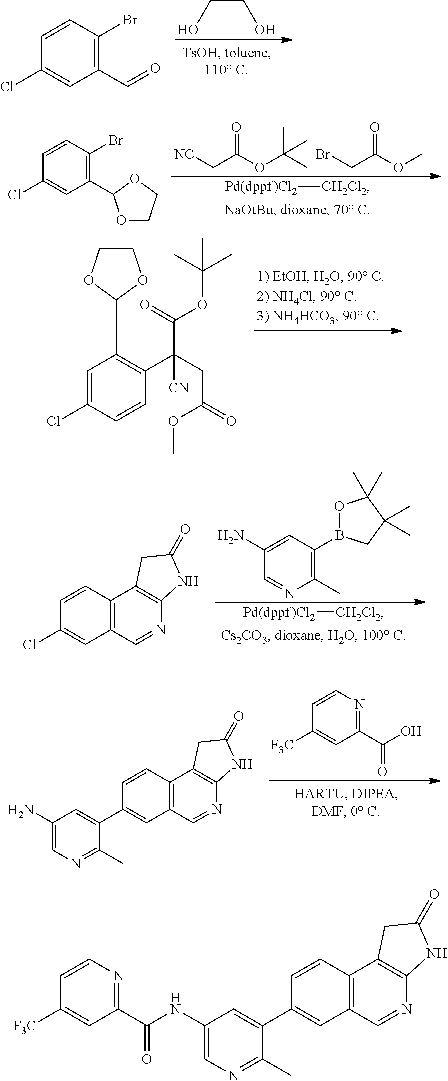

Step 1. To a solution of 2-bromo-5-chlorobenzaldehyde (5.5 g, 25 mmol) in toluene (250 mL) was added ethane-1,2-diol (3.1 g, 50 mmol) and 4-methylbenzenesulfonic acid (86 mg, 0.5 mmol). The resulting mixture was stirred at reflux for 18 h using Dean-Stark apparatus. The reaction mixture was cooled to room temperature and quenched with a saturated aqueous solution of sodium bicarbonate (100 mL). The mixture was extracted with ethyl acetate (100 mL×2). The combined organic phases were washed with brine (100 mL), dried over sodium sulfate, filtered, and concentrated. The residue was purified by flash chromatography (petroleum ether/ethyl acetate=5/1) to afford 2-(2-bromo-5-chlorophenyl)-1,3-dioxolane (4.7 g, 17.83 mmol, 71%) as a white solid. MS (ESI) m/z 265.0 [M+H]⁺

Step 2. A mixture of 2-(2-bromo-5-chlorophenyl)-1,3-dioxolane (2.0 g, 7.58 mmol), 1,1′-bis(diphenylphosphino)ferrocene-palladium(II)dichloride dichloromethane complex (106 mg, 0.13 mmol), sodium tert-butoxide (1.52 g, 15.8 mmol), and tert-butyl 2-cyanoacetate (892 mg, 6.32 mmol) in dioxane (30 mL) was stirred at 70° C. for 4 hours under nitrogen. After cooling to room temperature, methyl 2-bromoacetate (967 mg, 6.32 mmol) was added and the reaction mixture was stirred at 70° C. for 16 hours. The reaction was cooled to room temperature and quenched with a saturated aqueous solution of ammonium chloride (50 mL). The mixture was extracted with ethyl acetate (60 mL×3). The combined organic phases were washed with brine (60 mL), dried over sodium sulfate, filtered, and concentrated. The residue was purified by flash chromatography (ethyl acetate/petroleum ether=2/5) to afford 1-tert-butyl 4-methyl 2-(4-chloro-2-(1,3-dioxolan-2-yl)phenyl)-2-cyanosuccinate (1.4 g, 3.54 mmol, 56%) as a white solid. MS (ESI) m/z 418.0 [M+Na]⁺

Step 3. A mixture of 1-tert-butyl 4-methyl 2-(4-chloro-2-(1,3-dioxolan-2-yl)phenyl)-2-cyanosuccinate (198 mg, 0.5 mmol) in ethanol (6 mL) and water (4 mL) was stirred at 90° C. for 18 hours under nitrogen in a microwave tube. After cooling to room temperature, ammonium chloride (267 mg, 5.0 mmol) was added, and the reaction was heated to 90° C. and stirred for 3 hours. The mixture was then cooled to room temperature and a 1 M aqueous solution of ammonium bicarbonate (791 mg, 10.0 mmol) was added. The reaction mixture was stirred at 90° C. for 3 hours, then cooled to room temperature. The mixture was diluted with ethyl acetate (80 mL) and washed with water (50 mL). The organic phase was washed with brine (40 mL), dried over sodium sulfate, filtered, and concentrated. The residue was purified by flash chromatography (ethyl acetate/petroleum ether=4/5) to afford 7-chloro-1H-pyrrolo[2,3-c]isoquinolin-2(3H)-one (14 mg, 0.064 mmol, 13%) as a yellow solid. MS (ESI) m/z 219.1 [M+H]⁺

Step 4. A mixture of 7-chloro-1H-pyrrolo[2,3-c]isoquinolin-2(3H)-one (35 mg, 0.16 mmol), 6-methyl-5-(4,4,5,5-tetramethyl-1,3,2-dioxaborolan-2-yl)pyridin-3-amine (56 mg, 0.24 mmol), [1,1′-Bis(diphenylphosphino)ferrocene]dichloropalladium(II) (26 mg, 0.032 mmol) and potassium carbonate (156 mg, 0.4 mmol) in 1,4-dioxane (3 mL) and water (0.6 mL) was stirred at 100° C. for 48 hours under nitrogen. The mixture was cooled to room temperature and concentrated. The residue was purified by flash chromatography (silica, methanol/dichloromethane=5/100) to afford 7-(5-amino-2-methylpyridin-3-yl)-1H-pyrrolo[2,3-c]isoquinolin-2(3H)-one (18 mg, 0.062 mmol, 39%) as a yellow solid. MS (ESI) m/z 291.1 [M+H]⁺

Step 5. A solution of 7-(5-amino-2-methylpyridin-3-yl)-1H-pyrrolo[2,3-c]isoquinolin-2(3H)-one (18 mg, 0.062 mmol), 4-(trifluoromethyl)picolinic acid (12 mg, 0.062 mmol), 1-[bis(dimethylamino)methylene]-1H-1,2,3-triazolo[4,5-b]pyridinium 3-oxid hexafluorophosphate (35 mg, 0.093 mmol) and N,N-diisopropylethylamine (24 mg, 0.186 mmol) in N,N-dimethylformamide (2 mL) was stirred at 0° C. for 0.5 hour. The reaction mixture was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford N-(6-methyl-5-(2-oxo-2,3-dihydro-1H-pyrrolo[2,3-c]isoquinolin-7-yl)pyridin-3-yl)-4-(trifluoromethyl)picolinamide (4.5 mg, 0.0097 mmol, 16%) as a white solid. ¹H NMR (400 MHz, DMSO-d₆) δ 11.15 (s, 1H), 11.10 (s, 1H), 9.10-9.02 (m, 3H), 8.37 (s, 1H), 8.32 (d, J=2.4 Hz, 1H), 8.14-8.09 (m, 2H), 7.85-7.76 (m, 2H), 3.91 (s, 2H), 2.47 (s, 3H). MS (ESI) m/z 464.0 [M+H]⁺

### Example 221. Synthesis of 4-(2-cyanopropan-2-yl)-N-(4-methyl-3-(2-oxo-2,3-dihydro-1H-pyrrolo[2,3-c]isoquinolin-7-yl)phenyl)picolinamide (Compound 275)

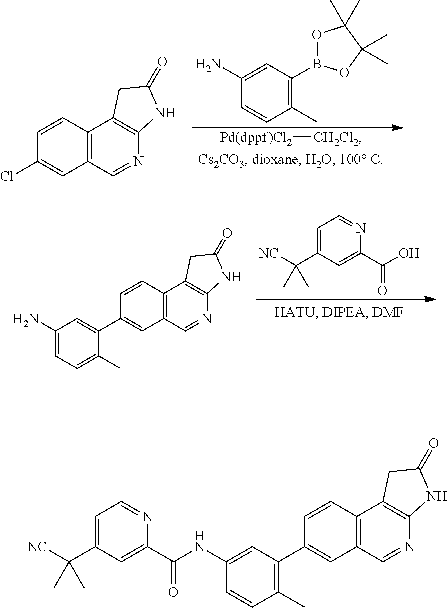

Step 1. A solution of 7-chloro-1H-pyrrolo[2,3-c]isoquinolin-2(3H)-one (80 mg, 0.37 mmol), 4-methyl-3-(4,4,5,5-tetramethyl-1,3,2-dioxaborolan-2-yl)aniline (103 mg, 0.44 mmol), 1,1′-bis(diphenylphosphino)ferrocene-palladium(II)dichloride (30 mg, 0.04 mmol) and cesium carbonate (358 mg, 1.10 mmol) in water (1 mL) and dioxane (5 mL) was stirred at 90° C. for 1 hour under argon. The mixture was diluted with ethyl acetate (50 mL) and washed with water (50 mL). The organic layer was concentrated and purified by flash chromatography (silica, petroleum ether) to give 7-(5-amino-2-methylphenyl)-1,3-dihydro-2H-pyrrolo[2,3-c]isoquinolin-2-one (40 mg, 0.14 mmol, 38%) as a yellow solid. MS (ESI) m/z 290.2 [M+H]⁺

Step 2. To a solution of 7-(5-amino-2-methylphenyl)-1,3-dihydro-2H-pyrrolo[2,3-c]isoquinolin-2-one (20 mg, 0.07 mmol) in N,N-dimethylformamide (2 mL) were added N,N-diisopropylethylamine (27 mg, 0.21 mmol), 1-[bis(dimethylamino)methylene]-1H-1,2,3-triazolo[4,5-b]pyridinium 3-oxid hexafluorophosphate (39 mg, 0.10 mmol) and 4-(2-cyanopropan-2-yl)picolinic acid (13 mg, 0.07 mmol). The mixture was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-70% in 15 min) to afford 4-(2-cyanopropan-2-yl)-N-(4-methyl-3-(2-oxo-2,3-dihydro-1H-pyrrolo[2,3-c]isoquinolin-7-yl)phenyl)picolinamide (2.1 mg, 0.005 mmol, 7%) as a yellow solid. ¹H NMR (500 MHz, DMSO-d₆) δ 1.79 (s, 6H), 2.29 (s, 3H), 4.61 (s, 2H), 7.35 (d, J=4.0 Hz, 1H), 7.72-7.83 (m, 5H), 7.99 (s, 1H), 8.35 (s, 1H), 8.74 (d, J=5.0 Hz, 1H), 8.98 (s, 1H). MS (ESI) m/z 462.1 [M+H]⁺

### Example 222. Synthesis of 2-(2-fluoropropan-2-yl)-N-(4-methyl-3-(2-oxo-2,3-dihydro-1H-pyrrolo[2,3-c]isoquinolin-7-yl)phenyl)isonicotinamide (Compound 276)

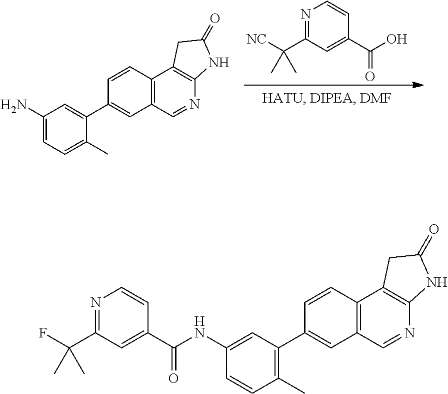 [SMILES: Cc1ccc(N)cc1-c1ccc2c3c(ncc2c1)NC(=O)C3 ; CC(C)(C#N)c1cc(C(=O)O)ccn1 ; Cc1ccc(NC(=O)c2ccnc(C(C)(C)F)c2)cc1-c1ccc2c3c(ncc2c1)NC(=O)C3]

To a solution of 7-(5-amino-2-methylphenyl)-1,3-dihydro-2H-pyrrolo[2,3-c]isoquinolin-2-one (30 mg, 0.10 mmol) in N,N-dimethylformamide (2 mL) were added N,N-diisopropylethylamine (40 mg, 0.31 mmol), 1-[bis(dimethylamino)methylene]-1H-1,2,3-triazolo[4,5-b]pyridinium 3-oxid hexafluorophosphate (59 mg, 0.16 mmol) and 4-(2-cyanopropan-2-yl)picolinic acid (19 mg, 0.10 mmol). The mixture was stirred at room temperature for 2 hours. The reaction was purified by prep-HPLC (Column: Welch Xtimate 21.2×250 mm C18, 10 μm, Mobile Phase: A: water (10 mM ammonium bicarbonate), B: acetonitrile; B %: 30%-700.% in 15 min) to afford 2-(2-fluoropropan-2-yl)-N-(4-methyl-3-(2-oxo-2,3-dihydro-1H-pyrrolo[2,3-c]isoquinolin-7-yl)phenyl)isonicotinamide (1.1 mg, 0.0052 mmol, 3%) as a yellow solid. ¹H NMR (400 MHz, DMSO-d₆) δ 1.69 (s, 3H), 1.75 (s, 3H), 2.29 (s, 3H), 3.93 (s, 2H), 7.35 (d, J=8.0 Hz, 1H), 7.66 (dd, J₁=2.0 Hz, J₂=8.0 Hz, 1H), 7.71-7.77 (m, 3H), 7.83 (d, J=8.4 Hz, 1H), 7.98 (s, 1H), 8.06 (s, 1H), 8.69 (d, J=5.2 Hz, 1H), 8.98 (s, 1H). MS (ESI) m/z 455.1 [M+H]⁺

### Example 223. Biological Activity of Exemplary Compounds

Retroviral Production: The BRAF-KIAA1549 fusion mutant was subcloned into pMXs-IRES-Blasticidin (RTV-016, Cell Biolabs, San Diego, CA). Retrovirus was produced by transfection of HEK 293T cells with the retroviral BRAF mutant expression vector pMXs-IRES-Blasticidin (RTV-016, Cell Biolabs), pCMV-Gag-Pol vector and pCMV-VSV-G-Envelope vector. Briefly, HEK 293T cells were plated in Poly-D-lysine coated 6-well plate (2.5×10⁵ cells per well) and incubated overnight. The next day, retroviral plasmids (1 μg of BRAF-KIAA fusion mutant, 0.32 μg of pCMV-Gag-Pol and 0.165 μg pCMV-VSV-G) were mixed in 300 μL of Optimem (31985, Life Technologies). The mixture was incubated at room temperature for 5 minutes and then added to Optimem containing transfection reagent Lipofectamine (11668, Invitrogen) and incubated for 20 minutes. The mixture was then added dropwise to HEK 293T cells. The next day the medium was replaced with fresh culture medium and retrovirus was harvested at 24 hours.

Generation of BRAF-KIAA1549 fusion stable cell line: BaF3 cells were plated in V-bottom 96 well plate (COSTAR #3894) in 80 μL of RPMI/10% FBS and 0.01 ng/mL mouse IL-3, supplemented with 8 μg/mL polybrene (4×10⁴ cells/well). Cells were transduced with 20 μL of BRAF-KIAA1549 fusion viral supernatant by centrifuging for 30 minutes at 1000 rpm. Cells were placed in a 37° C. incubator overnight. Next day 100 μL of RPMI/10% FBS and 0.01 ng/mL mouse IL-3 was added. After 24 hours, 100 μL of cells were transferred to a 24 well plate containing 900 μL of RPM/10% FBS and 15 μg/mL Blasticidin. Contents of the 24 well plate were sampled for cell viability via CellTiterGlo (Promega) over a 2-week period. Cells that were observed to have greater than 1-fold viability over day of plating were expanded into T25 flasks for cell banking and Sanger sequence confirmation.

Assay for cell proliferation: BaF3 BRAF-KIAA1549 fusion cells were resuspended at 1.5×10⁵ c/mL in RPMI containing 10% Heat Inactivated FBS, 1% L-glutamine and dispensed in duplicate (7×10⁴ c/well) into 384 well plates. To determine the effect of compound on cell proliferation, BaF3 BRAF-KIAA1549 fusion cells were incubated for 96 hours in the presence of vehicle control (DMSO) or compound at varying concentrations. Inhibition of cell growth was determined by luminescent quantification (Envision by Perkin Elmer) of intracellular ATP content using CellTiterGlo (Promega), according to the protocol provided by the manufacturer. To determine the IC₅₀ values, the vehicle-treated cells were normalized as viable cells and analyzed using the CDD Vault, Collaborative Drug Discovery, Burlingame, CA (the Levenberg-Marquardt algorithm; Levenberg, K., 1994; Marquardt, D., 1963).

Tables A1 and A2 assign each compound a code for potency in the BaF3 BRAF-KIAA1549 fusion cell proliferation assay: A, B, C, or D. According to the code, A represents an IC₅₀ value <20 nM; B represents an IC₅₀ value ≥20 nM and <100 nM; C represents an IC₅₀ value ≥100 nM and <500 nM; and D represents an IC₅₀ value ≥500 nM.

TABLE A1

| | BaF3- |
|---|---|
| Compound | BRAF: KIAA1549 |
| No. | fusion |
| 1 | A |
| 2 | B |
| 3 | A |
| 4 | A |
| 5 | B |
| 6 | A |
| 7 | A |
| 8 | B |
| 9 | A |
| 10 | A |
| 11 | B |
| 12 | B |
| 13 | C |
| 14 | A |
| 15 | B |
| 16 | A |
| 17 | A |
| 18 | A |
| 19 | A |
| 20 | A |
| 21 | B |
| 22 | B |
| 23 | B |
| 24 | A |
| 25 | B |
| 26 | B |
| 27 | A |
| 28 | C |
| 29 | A |
| 30 | B |
| 31 | B |
| 32 | A |
| 33 | A |
| 34 | B |
| 35 | B |
| 36 | C |
| 37 | B |
| 38 | B |
| 39 | B |
| 40 | B |
| 41 | B |
| 42 | B |
| 43 | B |
| 44 | B |
| 45 | C |
| 46 | C |
| 47 | C |
| 48 | D |
| 49 | A |
| 50 | C |
| 51 | D |
| 52 | A |
| 53 | B |
| 55 | A |
| 56 | B |
| 57 | A |
| 58 | B |
| 59 | B |
| 60 | A |
| 61 | C |
| 62 | B |
| 63 | B |
| 64 | D |
| 65 | B |
| 66 | B |
| 68 | B |
| 69 | C |
| 71 | B |
| 72 | D |
| 75 | B |
| 77 | B |
| 78 | B |
| 79 | A |
| 153 | B |
| 154 | A |
| 175 | B |
| 176 | B |

TABLE A2

| | BaF3- |
|---|---|
| Compound | BRAF: KIAA1549 |
| No. | fusion |
| 189 | C |
| 190 | C |
| 191 | B |
| 192 | C |
| 193 | A |
| 194 | B |
| 195 | D |
| 196 | D |
| 197 | D |
| 198 | D |
| 199 | D |
| 200 | A |
| 201 | B |
| 202 | B |
| 203 | A |
| 204 | A |
| 205 | C |
| 206 | B |
| 207 | D |
| 208 | D |
| 209 | D |
| 210 | D |
| 211 | D |
| 212 | D |
| 213 | D |
| 214 | B |
| 215 | B |
| 216 | C |
| 217 | B |
| 218 | B |
| 219 | D |
| 220 | B |
| 221 | B |
| 222 | B |
| 223 | B |
| 224 | C |
| 225 | B |
| 226 | B |
| 227 | B |
| 228 | C |
| 229 | B |
| 230 | B |
| 231 | B |
| 232 | C |
| 233 | B |
| 234 | B |
| 235 | B |
| 236 | B |
| 237 | B |
| 238 | B |
| 239 | D |
| 240 | D |
| 241 | C |
| 242 | B |
| 243 | B |
| 244 | D |
| 245 | B |
| 246 | D |
| 247 | B |
| 248 | B |
| 249 | B |
| 250 | D |
| 251 | C |
| 252 | B |
| 253 | B |
| 254 | C |
| 255 | B |
| 256 | B |
| 257 | B |
| 258 | B |
| 259 | B |
| 260 | B |
| 261 | A |
| 262 | B |
| 263 | A |
| 264 | B |
| 265 | A |
| 266 | B |
| 267 | A |
| 269 | C |
| 270 | D |
| 271 | B |
| 272 | B |
| 273 | C |
| 274 | A |
| 275 | B |
| 276 | A |

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound of Formula (0): 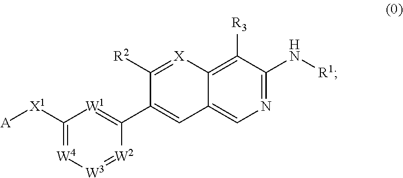
an isomer thereof, or a pharmaceutically acceptable salt thereof, wherein:
X is CR^{X} or N;
R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
R² is H, halogen, cyano, oxo, OH, C₁-C₆ alkyl, C₂-C₆ alkenyl, C₂-C₆ alkynyl, or C₁-C₆ alkoxy;
R³ is H, halogen, cyano, oxo, OH, C₁-C₆ alkyl, C₂-C₆ alkenyl, C₂-C₆ alkynyl, or C₁-C₆ alkoxy; or
R¹ and R³, together with the intervening atoms, form a 4- to 12-membered heterocycloalkyl optionally substituted with one or more oxo;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

2. A compound of Formula (I′): 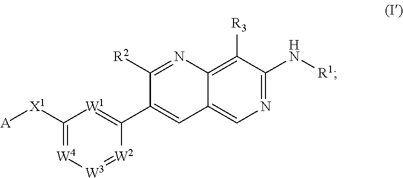
an isomer thereof, or a pharmaceutically acceptable salt thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl;
R² is H, halogen, cyano, oxo, OH, C₁-C₆ alkyl, C₂-C₆ alkenyl, C₂-C₆ alkynyl, or C₁-C₆ alkoxy;
R³ is H, halogen, cyano, oxo, OH, C₁-C₆ alkyl, C₂-C₆ alkenyl, C₂-C₆ alkynyl, or C₁-C₆ alkoxy; or
R¹ and R³, together with the intervening atoms, form a 4- to 12-membered heterocycloalkyl optionally substituted with one or more oxo;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NR^{X1})—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} independently is H, S(═O)₂R^{X1a}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 12-membered heterocycloalkyl, wherein the C₁-C₆ alkyl, or 3- to 12-membered heterocycloalkyl is optionally substituted with one or more halogen;
A is C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₁₂ cycloalkyl, 3- to 12-membered heterocycloalkyl, C₆-C₁₀ aryl, 5- to 10-membered heteroaryl, —(C₁-C₆ alkyl)-(C₃-C₁₂ cycloalkyl), —(C₁-C₆ alkyl)-(3- to 12-membered heterocycloalkyl), —(C₁-C₆ alkyl)-(C₆-C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₁₂ cycloalkyl, 3- to 12-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

3. A compound of Formula (I): 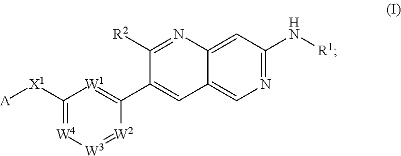
an isomer thereof, or a pharmaceutically acceptable salt thereof, wherein:
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
R² is H, cyano, oxo, OH, C₁-C₆ alkyl, C₂-C₆ alkenyl, C₂-C₆ alkynyl, or C₁-C₆ alkoxy;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C(—C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

4. The compound of claim 1, being of Formula (II′): 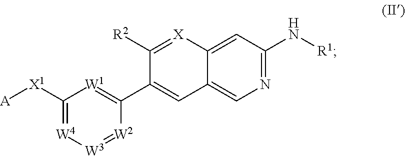
an isomer thereof, or a pharmaceutically acceptable salt thereof, wherein:
X is CR^{X} or N;
R^{X} is H, halogen, cyano, oxo, OH, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more halogen, cyano, oxo, or OH;
W¹ is N or CR^{W1};
R^{W1} is H, halogen, C₁-C₆ alkyl, C₂-C₆ alkenyl, or C₂-C₆ alkynyl;
W² is N or CR^{W2};
R^{W2} is H, halogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl is optionally substituted with one or more halogen;
W³ is N or CR^{W3};
R^{W3} is H, halogen, C₁-C₆ alkyl, C₂-C₆ alkenyl, or C₂-C₆ alkynyl;
W⁴ is N or CR^{W4};
R^{W4} is H, halogen, C₁-C₆ alkyl, C₂-C₆ alkenyl, C₂-C₆ alkynyl, or S(C₁-C₆ alkyl);
R¹ is H, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{1a};
each R^{1a} independently is halogen, cyano, oxo, OH, NH₂, NHC(═O)O(C₁-C₆ alkyl), N(C₁-C₆ alkyl)₂, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl;
R² is H, cyano, oxo, OH, C₁-C₆ alkyl, C₂-C₆ alkenyl, C₂-C₆ alkynyl, or C₁-C₆ alkoxy;
X¹ is —NR^{X1}—*, —C(═O)NR^{X1}—*, —NR^{X1}C(═O)—*, —NR^{X1}C(═O)O—*, —NR^{X1}N═C—*, —NR^{X1}C(═NH)—*, —NR^{X1}C(═NH)NR^{X1}—*, —NR^{X1}C(═O)NR^{X1}—*, —S(═O)₂NR^{X1}—*, or —NR^{X1}S(═O)₂—*, wherein * denotes attachment to A;
R^{X1} is H, S(═O)₂R^{X1a}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{X1a};
each R^{X1a} independently is halogen, C₁-C₆ alkyl, or 3- to 8-membered heterocycloalkyl, wherein the C₁-C₆ alkyl, or 3- to 8-membered heterocycloalkyl is optionally substituted with one or more halogen;
A is C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C₆-C₁₀ aryl), or —(C₁-C₆ alkyl)-(5- to 10-membered heteroaryl), wherein the C₁-C₆ alkyl, C₃-C₈ cycloalkyl, 3- to 8-membered heterocycloalkyl, C₆-C₁₀ aryl, 5- to 10-membered heteroaryl, —(C₁-C₆ alkyl)-(C₃-C₈ cycloalkyl), —(C₁-C₆ alkyl)-(3- to 8-membered heterocycloalkyl), —(C₁-C₆ alkyl)-(C(—C₁₀ aryl), or —(C₁-C₆ alkyl)-(5- to 10-membered heteroaryl) is optionally substituted with one or more R^{A};
each R^{A} independently is halogen, cyano, oxo, OH, OR^{A1}, NH₂, NHR^{A1}, N(R^{A1})₂, (═N)R^{A1}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A1};
each R^{A1} independently is halogen, cyano, oxo, OH, OR^{A2}, NH₂, NHR^{A2}, N(R^{A2})₂, C(═O)R^{A2}, C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl, wherein the C₁-C₆ alkyl, C₂-C₆ alkenyl, C₂-C₆ alkynyl, C₁-C₆ alkoxy, C₃-C₈ cycloalkyl, 3- to 8-membered heterocycloalkyl, C₆-C₁₀ aryl, or 5- to 10-membered heteroaryl is optionally substituted with one or more R^{A2}; and
each R^{A2} independently is halogen, cyano, OH, NH₂, N(R^{A3})₂, C(═O)R^{A3}, C₁-C₆ alkyl, C₂-C₆ alkenyl, or C₂-C₆ alkynyl,
wherein R^{A3} is C₁-C₆ alkyl, C₂-C₆ alkenyl, or C₂-C₆ alkynyl.

5. The compound of any one of the preceding claims, wherein:
W¹ is CR^{W1};
R^{W1} is H, halogen, or C₁-C₆ alkyl;
W² is N or CR^{W2};
R^{W2} is H, halogen, or C₁-C₆ alkyl;
W³ is N or CR^{W3};
R^{W3} is H, or halogen;
W⁴ is N or CR^{W4};
R^{W4} is H, or halogen;
R¹ is H, or C₁-C₆ alkyl, wherein the C₁-C₆ alkyl is optionally substituted with one or more R^{1a};
each R^{1a} is cyano;
R² is H, or cyano;
X¹ is —NR^{X1}*, —C(═O)NR^{X1}—*, —NR^{X1}C(═O)—*, or —NR^{X1}C(═NH)—*, wherein * denotes attachment to A;
R^{X1} is H;
A is C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl), wherein the C₆-C₁₀ aryl, 5- to 10-membered heteroaryl, —(C₁-C₆ alkyl)-(3- to 8-membered heterocycloalkyl), or —(C₁-C₆ alkyl)-(5- to 10-membered heteroaryl) is optionally substituted with one or more R^{A};
each R^{A} independently is halogen, OH, OR^{A1}, NHR^{A1}, N(R^{A1})₂, C₁-C₆ alkyl, C₁-C₆ alkoxy, or C₃-C₈ cycloalkyl, wherein the C₁-C₆ alkyl, C₁-C₆ alkoxy, or C₃-C₈ cycloalkyl is optionally substituted with one or more R^{A1};
each R^{A1} independently is halogen, cyano, oxo, OH, OR^{A2}, NH₂, or C₁-C₆ alkyl, wherein the C₁-C₆ alkyl is optionally substituted with one or more R^{A2}; and
each R^{A2} independently is halogen, OH, or C₁-C₆ alkyl.

6. The compound of any one of the preceding claims, wherein R¹ is H, C₁-C₆ alkyl, C₂-C₆ alkenyl, C₂-C₆ alkynyl, or C₁-C₆ alkoxy, wherein the C₁-C₆ alkyl, C₂-C₆ alkenyl, C₂-C₆ alkynyl, or C₁-C₆ alkoxy is optionally substituted with one or more R^{1a}.

7. The compound of any one of the preceding claims, wherein R^{1a} is cyano.

8. The compound of any one of the preceding claims, wherein R² is H or cyano.

9. The compound of any one of the preceding claims, wherein R³ is H or cyano.

10. The compound of any one of the preceding claims, wherein X¹ is —NR^{X1}—*, —NR^{X1}C(═O)—*, —C(═O)NR^{X1}—*, or —NR^{X1}C(═NH)—*, wherein * denotes attachment to A.

11. The compound of any one of the preceding claims, wherein R^{X1} is H or C₁-C₆ alkyl optionally substituted with one or more R^{X1a}.

12. The compound of any one of the preceding claims, wherein R^{X1a} is halogen, C₁-C₆ alkyl, or 3- to 8-membered heterocycloalkyl, wherein the C₁-C₆ alkyl, or 3- to 8-membered heterocycloalkyl is optionally substituted with one or more halogen.

13. The compound of any one of the preceding claims, wherein A is C₆-C₁₀ aryl, 5- to 10-membered heteroaryl, —(C₁-C₆ alkyl)-(5- to 10-membered heteroaryl), or —(C₁-C₆ alkyl)-(3- to 12-membered heterocycloalkyl), wherein the C₆-C₁₀ aryl, 5- to 10-membered heteroaryl, —(C₁-C₆ alkyl)-(5- to 10-membered heteroaryl), or —(C₁-C₆ alkyl)-(3- to 12-membered heterocycloalkyl) is optionally substituted with one or more R^{A}.

14. The compound of any of the preceding claims, wherein R^{A} is halogen, cyano, OH, O(R^{A1}), NHR^{A1}, N(R^{A1})₂, C₁-C₆ alkyl, C₁-C₆ alkoxy, or C₃-C₁₂ cycloalkyl, wherein the C₁-C₆ alkyl, C₁-C₆ alkoxy, or C₃-C₁₂ cycloalkyl is optionally substituted with one or more R^{A1}.

15. The compound of any of the preceding claims, wherein R^{A1} is halogen, cyano, oxo, OH, OR^{A2}, NH₂, or C₁-C₆ alkyl optionally substituted with one or more R^{A2}.

16. The compound of any of the preceding claims, wherein R^{A2} is halogen, OH, or C₁-C₆ alkyl.

17. The compound of any of the preceding claims, wherein the compound is of Formula (I-a), (I-b), (I-c), (I-d), (I-e), or (I-f): 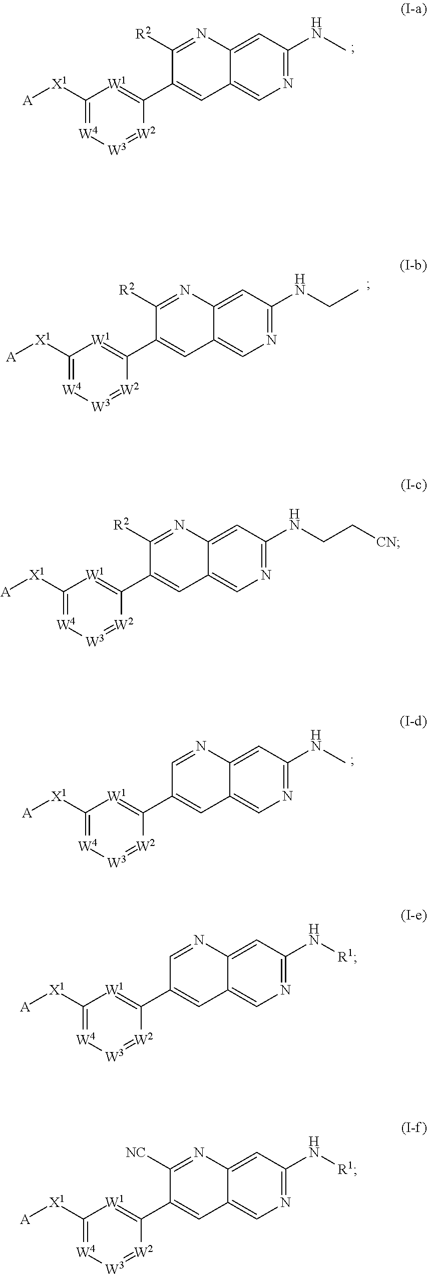
or a pharmaceutically acceptable salt or stereoisomer thereof.

18. The compound of any of the preceding claims, wherein the compound is of Formula (I-g), (I-h), (I-i), (I-j), (I-k), (I-l), (I-m), (I-n), (I-o), (I-p), (I-q), (I-r), (I-s), or (I-t): 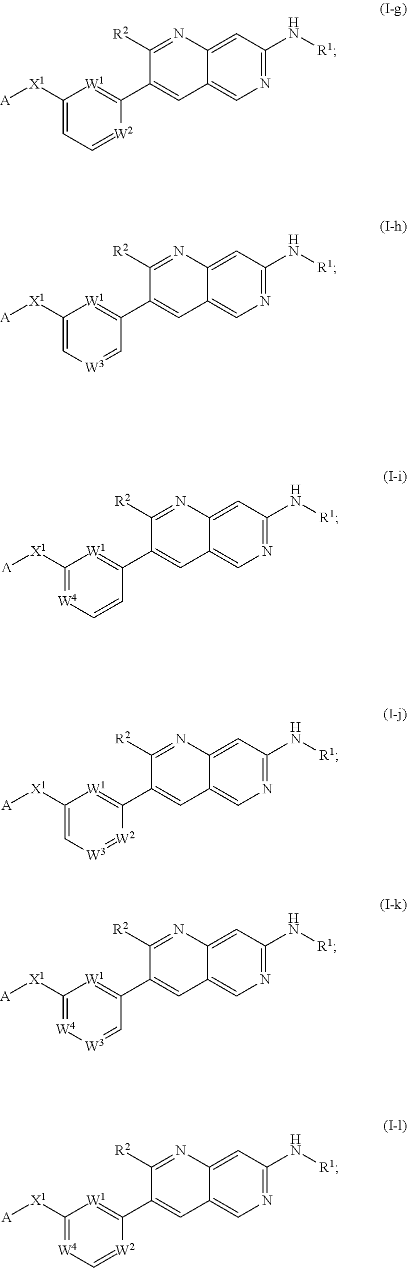 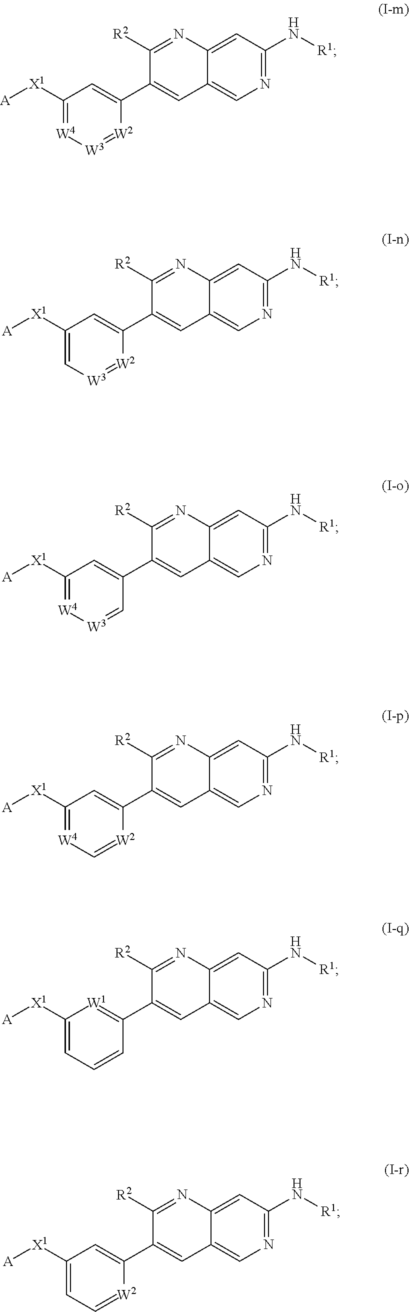 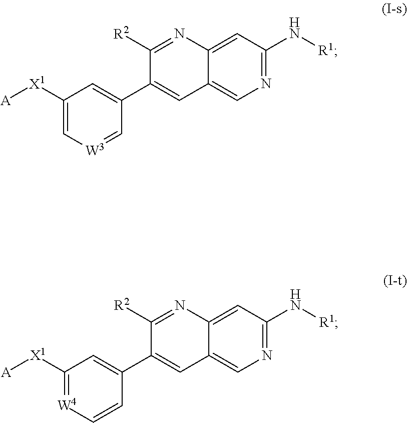
or a pharmaceutically acceptable salt or stereoisomer thereof.

19. The compound of any of the preceding claims, wherein the compound is of Formula (I-u), (I-v), (I-w), or (I-x): 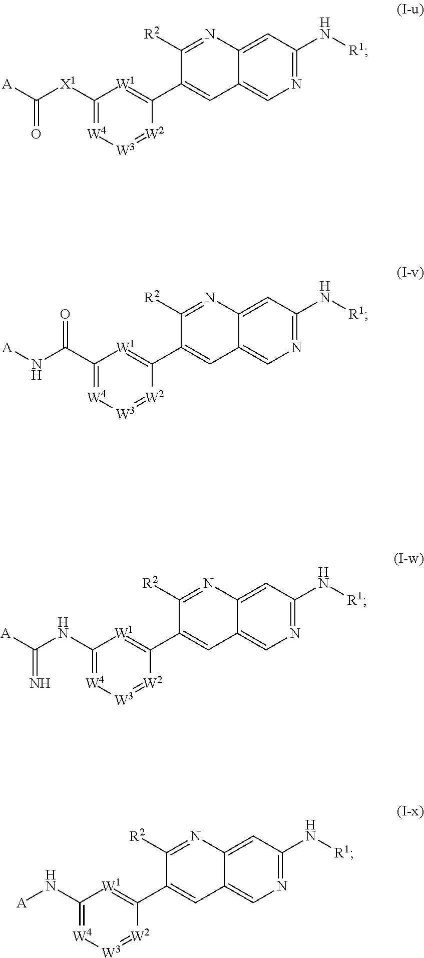
or a pharmaceutically acceptable salt or stereoisomer thereof.

20. The compound of any of the preceding claims, wherein the compound is selected from the compounds described in Table I or Table II, or a pharmaceutically acceptable salt or stereoisomer thereof.

21. A pharmaceutical composition comprising the compound of any one of the preceding claims and one or more pharmaceutically acceptable carriers or excipients.

22. A method of treating or preventing cancer in a subject, the method comprising administering to the subject a compound of any one of the preceding claims.

23. The compound of any one of the preceding claims for treating or preventing cancer in a subject.

24. Use of the compound of any one of the preceding claims in the manufacture of a medicament for treating or preventing cancer in a subject.

25. The method, compound, or use of any one of the preceding claims, wherein the cancer is characterized by at least one oncogenic mutation in the BRAF gene.

26. The method, compound, or use of any one of the preceding claims, wherein the cancer is characterized by at least one oncogenic variant of B-Raf.

27. The method, compound, or use of any one of the preceding claims, wherein the subject has at least one oncogenic mutation in the BRAF gene.

28. The method, compound, or use of any one of the preceding claims, wherein the subject has at least one tumor and/or cancerous cell that expresses an oncogenic variant of B-Raf.

29. The method, compound, or use of any one of the preceding claims, wherein the oncogenic mutation is a class I, a class II or a class III mutation.

30. The method, compound, or use of any one of the preceding claims, wherein the oncogenic variant of B-Raf can be any of the B-Raf variants put forth in Table 1b.

31. The method, compound, or use of any one of the preceding claims, wherein the cancer is a hematological cancer, solid cancer, melanoma, breast cancer, head and neck cancer, esophagogastric cancer, stomach and small intestine cancer, lung cancer, mesothelioma, hepatobiliary cancer, pancreatic cancer, kidney cancer, colorectal cancer, endometrial cancer, cervical cancer, ovarian cancer, bladder cancer, prostate cancer, soft tissue sarcoma, CNS and brain cancer, thyroid cancer, non-small cell lung cancer (NSCLC), colorectal cancer, melanoma, thyroid cancer, histiocytosis, small bowel cancer, gastrointestinal neuroendocrine cancer, carcinoma of unknown primary, non-melanoma skin cancer, prostate cancer, gastric cancer, non-Hodgkin's lymphoma, papillary thyroid carcinoma or glioblastoma.


SEQUENCE LISTING (1 sequence)

SEQ ID NO: 1 - protein, 766 aa, Homo sapiens [sequence omitted]